(19) United States
(12) Patent Application Publication
(10) Pub. No.: US 2019/0336504 A1
(43) Pub. Date: Nov. 7, 2019

(54) TREATMENT AND PREVENTION OF CYTOKINE RELEASE SYNDROME USING A CHIMERIC ANTIGEN RECEPTOR IN COMBINATION WITH A KINASE INHIBITOR

(71) Applicants: Saar Gill, (US); Saad Kenderian, (US); Marco Ruella, (US); Novartis AG, Basel (CH); The Trustees of the University of Pennsylvania, Philadelphia, PA (US)

(72) Inventors: Saar Gill, Philadelphia, PA (US); Saad Kenderian, Philadelphia, PA (US); Marco Ruella, Ardmore, PA (US)

(21) Appl. No.: 16/317,943

(22) PCT Filed: Jul. 14, 2017
(86) PCT No.: PCT/US2017/042129

§ 371 Date: Jan. 15, 2019

Related U.S. Application Data
(60) Provisional application No. 62/362,659, filed on Jul. 15, 2016.
(60) Provisional application No. 62/366,997, filed on Jul. 26, 2016.
(60) Provisional application No. 62/381,230, filed on Aug. 30, 2016.

(51) Int. Cl. A61K 31/519; A61P 37/02; A61K 39/395; A61K 35/17; A61P 35/00

(52) U.S. Cl. CPC A61K 31/519; A61P 37/02; A61K 2039/505; A61K 35/17; A61P 35/00; A61K 39/39558

(57) ABSTRACT

The disclosure provides compositions and methods for treating diseases associated with expression of an antigen or for treating or prevent cytokine release syndrome, e.g., by administering a CAR therapy with a kinase inhibitor, e.g., JAK-STAT inhibitor and/or BTK inhibitor.

This application claims priority to U.S. Ser. No. 62/362,659 filed Jul. 15, 2016, U.S. Ser. No. 62/366,997 filed Jul. 26, 2016, and U.S. Ser. No. 62/381,230 filed Aug. 30, 2016, the contents of all of which are incorporated herein by reference in their entireties.

### FIELD OF THE INVENTION

The present invention relates generally to the use of immune effector cells (e.g., T cells or NK cells) engineered to express a Chimeric Antigen Receptor (CAR), in combination with a kinase inhibitor (e.g., a JAK-STAT or a BTK inhibitor), to treat a disease and/or prevent cytokine release syndrome (CRS).

### BACKGROUND OF THE INVENTION

Many patients with hematological malignancies (e.g., B cell malignancies) are incurable with standard therapy. In addition, traditional treatment options often have serious side effects. Recent developments using chimeric antigen receptor (CAR) modified autologous T cell (CART) therapy, which relies on redirecting T cells to a suitable cell-surface molecule on cancer cells such as B cell malignancies, show promising results in harnessing the power of the immune system to treat B cell malignancies and other cancers (see, e.g., Sadelain et al., Cancer Discovery 3:388-398 (2013)). The clinical results of the murine derived CART19 (i.e., “CTL019”) have shown promise in establishing complete remissions in patients suffering with CLL as well as in childhood ALL (see, e.g., Kalos et al., Sci Transl Med 3:95ra73 (2011), Porter et al., NEJM 365:725-733 (2011), Grupp et al., NEJM 368:1509-1518 (2013)). Besides the ability for the chimeric antigen receptor on the genetically modified T cells to recognize and destroy the targeted cells, a successful therapeutic T cell therapy needs to have the ability to proliferate and persist over time, and to further monitor for leukemic cell escape. The variable quality of T cells whether it's a result of anergy, suppression or exhaustion will have effects on CAR-transformed T cells' performance but for which skilled practitioners have limited control over at this time. To be effective, CAR transformed patient T cells need to persist and maintain the ability to proliferate in response to the target antigen. It has been shown that ALL patient T cells perform can do this with CART19 comprising a murine scFv (see, e.g., Grupp et al., NEJM 368:1509-1518 (2013)).

Cytokine release syndrome (CRS) is a serious and common adverse side effect of immune cell-based therapies, e.g., CAR T cell treatment. Severe CRS is a potentially life-threatening toxicity. Deaths with severe cases of CRS have been reported. Diagnosis and management of CRS in response to immune cell-based therapies is routinely based on clinical parameters and symptoms, e.g., see CRS grading scale as described by Lee, D. et al. (2014) Blood 124(2):188-195. While the interleukin-6 receptor blocker tocilizumab and steroids can reverse CRS, concerns remain that these approaches may impair the anti-tumor effects. Also, there is a lack of preclinical models for CRS after human CART. There is a need for preclinical models for CRS after human CART administration. Also, there is a need for CRS prevention modalities—such modalities would enhance the clinical feasibility of CART therapy.

### SUMMARY OF THE INVENTION

The present disclosure is based, at least in part, on the discovery that a JAK-STAT kinase inhibitor, such as ruxolitinib, can ameliorate cytokine release syndrome (CRS) severity or prevent CRS after CART cell therapy for hematological cancers, such as acute myeloid leukemia (AML), without significantly impairing anti-tumor effect of the CART therapy. The present disclosure is also based, at least in part, on the discovery that a BTK inhibitor, such as ibrutinib, can improve or prevent CRS after a CD19 CAR therapy for B cell neoplasms. Additionally, the disclosure is based, at least in part, on the discovery that an IL-6 inhibitor (e.g., which can be used for CRS prevention/treatment) can be administered in combination with (e.g., before, concurrently, or after) a CAR therapy, without decreasing the anti-cancer efficacy of the CAR therapy.

Without wishing to be bound by theory, treating a subject having a disease described herein, e.g., a cancer described herein, with a combination therapy that includes a CAR-expressing cell and a JAK-STAT or BTK inhibitor is believed to result in improved inhibition or reduction of tumor progression and/or reduced adverse effects (e.g., reduced CRS) in the subject, e.g., as compared to treating a subject having the disease with the CAR-expressing cell or the JAK-STAT or BTK inhibitor alone.

Accordingly, the disclosure features, at least in part, compositions and methods of treating disorders such as cancer (e.g., hematological cancers or other B-cell malignancies) using immune effector cells (e.g., T cells or NK cells) that express a Chimeric Antigen Receptor (CAR) molecule (e.g., a CAR that binds to a B-cell antigen, e.g., CD123 or Cluster of Differentiation 19 protein (CD19) (e.g., OMIM Acc. No. 107265, Swiss Prot. Acc No. P15391)). The compositions include, and the methods include administering, immune effector cells (e.g., T cells or NK cells) expressing a CAR (e.g., a B cell targeting CAR), in combination with a kinase inhibitor (e.g., one or more of a JAK-STAT inhibitor and/or a BTK inhibitor). In some embodiments, the combination maintains, has better clinical effectiveness, and/or has lower toxicity (e.g., due to prevention of CRS) as compared to either therapy alone. In some embodiments, the subject is at risk of, or has, CRS; or the subject has been identified as having or at risk of developing CRS.

The disclosure further pertains to the use of engineered cells, e.g., immune effector cells (e.g., T cells or NK cells), to express a CAR molecule that binds to an antigen (e.g., tumor antigen described herein, e.g., a B-cell antigen, e.g., CD123 or CD19, in combination with a kinase inhibitor (e.g., at least one JAK-STAT inhibitor) to treat a disorder associated with expression of a B-cell antigen, e.g., CD123 or CD19 (e.g., a cancer, e.g., a hematological cancer).

Also provided herein are compositions and methods for preventing CRS in a subject by using a combination of a JAK-STAT inhibitor with a CAR-expressing cell (e.g., a B cell targeting CAR-expressing cell, e.g., CD123 CAR-expressing cell).

Also provided are compositions and methods for preventing CRS in a subject by using a combination of a BTK inhibitor with a CAR-expressing cell (e.g., B cell targeting CAR-expressing cell, e.g., a CD19 CAR-expressing cell), e.g., where the subject is at risk of, or has, CRS; or the subject has been identified as having or at risk of developing CRS.

In an aspect, provided herein is a method of treating a subject, e.g., a mammal, having a disease associated with expression of an antigen, e.g., tumor antigen, e.g., tumor antigen described herein. The method comprises administering to the subject an effective amount of a cell e.g., an immune effector cell (e.g., a T cell or NK cell) that expresses a CAR molecule that binds the antigen (e.g., antigen described herein, e.g., tumor antigen, e.g., B-cell antigen), in combination with a JAK-STAT inhibitor, e.g., a JAK-STAT inhibitor described herein, e.g., ruxolitinib.

In another aspect provided herein is a method of providing anti-tumor immunity to a subject, e.g., mammal, having a disease associated with expression of an antigen, e.g., tumor antigen, e.g., tumor antigen described herein. The method comprises administering to the subject an effective amount of a cell e.g., an immune effector cell (e.g., a T cell or NK cell) that expresses a CAR molecule that binds the antigen (e.g., antigen described herein, e.g., tumor antigen, e.g., B-cell antigen), in combination with a JAK-STAT inhibitor, e.g., a JAK-STAT inhibitor described herein, e.g., ruxolitinib.

In one embodiment, the CAR molecule binds to CD123, e.g., a CAR molecule that binds CD123 described herein.

In another aspect, provided herein is a method of treatment and/or preventing cytokine release syndrome (CRS), e.g., CRS associated with a CAR therapy (e.g., a CAR-expressing cell described herein) in a subject in need thereof, comprising administering a JAK-STAT inhibitor (e.g., ruxolitinib), alone or in combination with the CAR therapy, to the subject, thereby treating and/or preventing CRS in the subject.

In embodiments, the subject is at risk of developing, has, or is diagnosed with CRS. In embodiments, the subject has been, is being, or will be administered a CAR therapy, e.g., a CAR-expressing cell described herein.

In embodiments, the method further comprises administering an IL-6 inhibitor (e.g., an anti-IL6 receptor inhibitor, e.g., tocilizumab) to the subject. In embodiments, the method comprises administering to the subject (i) a JAK-STAT inhibitor (e.g., ruxolitinib), (ii) a CAR therapy (e.g., CAR-expressing cell described herein), and (iii) an IL-6 inhibitor (e.g., an anti-IL6 receptor inhibitor, e.g., tocilizumab).

In another aspect, provided herein is a method of preventing cytokine release syndrome (CRS) (e.g., CRS associated with a CAR therapy, e.g., B cell antigen CAR therapy, e.g., CD19 CAR therapy) in a subject in need thereof, comprising administering a BTK inhibitor (e.g., ibrutinib), alone or in combination with the CAR therapy, to the subject, thereby preventing CRS in the subject.

In embodiments, the subject is at risk of developing, has, or is diagnosed with CRS. In embodiments, the subject has been, is being, or will be administered a CAR therapy, e.g., a CAR therapy described herein. In embodiments, the subject is identified or has previously been identified as at risk for CRS.

In embodiments, the method comprises selecting the subject for administration of the BTK inhibitor. In embodiments, the subject is selected based on (i) his or her risk of developing CRS, (ii) his or her diagnosis of CRS, and/or (iii) whether he or she has been, is being, or will be administered a CAR therapy (e.g., a CAR therapy described herein, e.g., CAR19 therapy, e.g., CTL019). In embodiments, the subject is selected for administration of the BTK inhibitor if the subject is diagnosed with CRS, e.g., severe or non-severe CRS. In embodiments, the subject is selected for administration of the BTK inhibitor if the subject is at risk of (e.g., identified as at risk of) developing CRS. In embodiments, the subject is selected for administration of the BTK inhibitor if the subject has been, is being, or will be administered a CAR therapy (e.g., a CAR therapy described herein, e.g., CAR19 therapy, e.g., CTL019).

In embodiments, the method further comprises administering an IL-6 inhibitor (e.g., an anti-IL6 receptor inhibitor, e.g., tocilizumab) to the subject. In embodiments, the method comprises administering to the subject (i) a BTK inhibitor (e.g., ibrutinib), (ii) a CAR therapy (e.g., CAR-expressing cell described herein), and (iii) an IL-6 inhibitor (e.g., an anti-IL6 receptor inhibitor, e.g., tocilizumab).

In yet another aspect, provided herein is a method of treating or preventing CRS associated with administration of a cell, e.g., a population of cells, expressing a CAR in a subject.

In yet another aspect, provided herein is a method of treating or preventing CRS associated with administration of a T cell inhibitor therapy, e.g., a CD19-inhibiting or depleting therapy, e.g., a therapy that includes a CD19 inhibitor. In embodiments, the CD19-inhibiting or depleting therapy is associated with CRS.

The method of treating or preventing CRS comprising administering to the subject an IL-6 inhibitor (e.g., an anti-IL6 receptor inhibitor, e.g., tocilizumab) prior to, simultaneously with, or within 1 day (e.g, within 24 hours, 12 hours, 6 hours, 5, hours, 4 hours, 3 hours, 2 hours, 1 hour or less) of, administration of a dose (e.g., a first dose) of said cell, e.g., said population of cells, expressing a CAR, or said therapy.

In embodiments, the IL-6 inhibitor (e.g., tocilizumab) is administered upon (e.g., within 1 hour, 30 minutes, 20 minutes, 15 minutes or less) a first sign of a symptom of CRS (e.g., a fever, e.g., characterized by a temperature of at least 38° C. (e.g., at least 38.5° C.), e.g., for two successive measurements in 24 hours (e.g., at least 4, 5, 6, 7, 8 hours, or more, apart)) in the subject.

The following embodiments pertain to any methods and compositions described herein.

### CAR Molecules

In embodiments, the CAR molecule comprises an antigen binding domain (e.g., B cell antigen binding domain, CD123 binding domain, or CD19 binding domain), transmembrane domain, and an intracellular signaling domain (e.g., an intracellular signaling domain comprising a costimulatory domain and/or a primary signaling domain).

In embodiments, the CAR comprises an antigen binding domain that binds one or more of the following: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

In other embodiment, the CAR molecule is capable of binding an antigen described herein, e.g., an antigen described in the Antigens section below.

In one embodiment, the antigen comprises a B cell antigen, e.g., CD10, CD19, CD20, CD22, CD34, CD123, FLT-3, ROR1, CD79b, CD179b, and/or CD79a.

In embodiments, the antigen is CD123. In embodiments, the antigen is CD19.

In other embodiments, the antigen is BCMA. In embodiments, the antigen is CLL.

### Exemplary CAR Molecules

In an embodiment, the CAR molecule comprises a CD123 CAR described herein, e.g., a CD123 CAR described in US2014/0322212A1 or US2016/0068601A1, both incorporated herein by reference. In embodiments, the CD123 CAR comprises an amino acid, or has a nucleotide sequence shown in US2014/0322212A1 or US2016/0068601A1, both incorporated herein by reference.

In embodiments, the CAR molecule comprises a CD19 CAR molecule described herein, e.g., a CD19 CAR molecule described in US-2015-0283178-A1, e.g., CTL019. In embodiments, the CD19 CAR comprises an amino acid, or has a nucleotide sequence shown in US-2015-0283178-A1, incorporated herein by reference.

In one embodiment, CAR molecule comprises a BCMA CAR molecule described herein, e.g., a BCMA CAR described in US-2016-0046724-A1. In embodiments, the BCMA CAR comprises an amino acid, or has a nucleotide sequence shown in US-2016-0046724-A1, incorporated herein by reference.

In an embodiment, the CAR molecule comprises a CLL1 CAR described herein, e.g., a CLL1 CAR described in US2016/0051651A1, incorporated herein by reference. In embodiments, the CLL1 CAR comprises an amino acid, or has a nucleotide sequence shown in US2016/0051651A1, incorporated herein by reference.

In an embodiment, the CAR molecule comprises a CD33 CAR described herein, e.ga CD33 CAR described in US2016/0096892A1, incorporated herein by reference. In embodiments, the CD33 CAR comprises an amino acid, or has a nucleotide sequence shown in US2016/0096892A1, incorporated herein by reference.

In an embodiment, the CAR molecule comprises an EGFRvIII CAR molecule described herein, e.g., an EGFRvIII CAR described US2014/0322275A1, incorporated herein by reference. In embodiments, the EGFRvIII CAR comprises an amino acid, or has a nucleotide sequence shown in US2014/0322275A1, incorporated herein by reference.

In an embodiment, the CAR molecule comprises a mesothelin CAR described herein, e.g., a mesothelin CAR described in WO 2015/090230, incorporated herein by reference. In embodiments, the mesothelin CAR comprises an amino acid, or has a nucleotide sequence shown in WO 2015/090230, incorporated herein by reference.

### CD123 CAR Antigen Binding Domains

In embodiments, the CAR molecule is capable of binding CD123 (e.g., wild-type or mutant CD123). In embodiments, the CAR molecule comprises an anti-CD123 binding domain comprising one or more (e.g., all three) light chain complementary determining region 1 (LC CDR1), light chain complementary determining region 2 (LC CDR2), and light chain complementary determining region 3 (LC CDR3) of an anti-CD123 binding domain described herein (e.g., described in US2014/0322212A1 or US2016/0068601A1), and/or one or more (e.g., all three) heavy chain complementary determining region 1 (HC CDR1), heavy chain complementary determining region 2 (HC CDR2), and heavy chain complementary determining region 3 (HC CDR3) of an anti-CD123 binding domain described herein (e.g., described in US2014/0322212A1 or US2016/0068601A1), e.g., an anti-CD123 binding domain comprising one or more, e.g., all three, LC CDRs and one or more, e.g., all three, HC CDRs.

In one embodiment, the encoded CD123 binding domain comprises one or more (e.g., all three) light chain complementary determining region 1 (LC CDR1), light chain complementary determining region 2 (LC CDR2), and light chain complementary determining region 3 (LC CDR3) of a CD123 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a CD123 binding domain described herein, e.g., a CD123 binding domain comprising one or more, e.g., all three, LC CDRs and one or more, e.g., all three, HC CDRs. In one embodiment, the encoded CD123 binding domain (e.g., a human or humanized CD123 binding domain) comprises a light chain variable region described herein (e.g., in Tables 11A, 12A or 12B) and/or a heavy chain variable region described herein (e.g., in Tables 11A, 12A or 12B). In one embodiment, the encoded CD123 binding domain is a scFv comprising a light chain and a heavy chain of an amino acid sequence of Tables 11A, 12A or 12B. In an embodiment, the CD123 binding domain (e.g., an scFv) comprises: a light chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a light chain variable region provided in Tables 11A, 12A or 12B, or a sequence with at least 95%, e.g., 95-99%, identity with an amino acid sequence of Tables 11A, 12A or 12B; and/or a heavy chain variable region comprising an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of a heavy chain variable region provided in Tables 11A, 12A or 12B, or a sequence at least 95% (e.g., 95-99%) identity to an amino acid sequence of Tables 11A, 12A or 12B.

In other embodiments, the encoded CD123 binding domain comprises a HC CDR1, a HC CDR2, and a HC CDR3 of any CD123 heavy chain binding domain amino acid sequences listed in Table 11A, 12A or 12B. In embodiments, the CD33 binding domain further comprises a LC CDR1, a LC CDR2, and a LC CDR3. In embodiments, the CD123 binding domain comprises a LC CDR1, a LC CDR2, and a LC CDR3 of any CD123 light chain binding domain amino acid sequences listed in Table 11A, 12A or 12B.

In some embodiments, the encoded CD123 binding domain comprises one, two or all of LC CDR1, LC CDR2, and LC CDR3 of any CD123 light chain binding domain amino acid sequences listed in Table 11A or 12B, and one, two or all of HC CDR1, HC CDR2, and HC CDR3 of any CD123 heavy chain binding domain amino acid sequences listed in Table 11A, 12A or 12B.

In one embodiment, the encoded CD123 binding domain comprises an amino acid sequence selected from a group consisting of SEQ ID NO:157-160, 184-215, 478, 480, 483, and 485. In an embodiment, the encoded CD123 binding domain (e.g., an scFv) comprises an amino acid sequence having at least one, two or three modifications (e.g., substitutions, e.g., conservative substitutions) but not more than 30, 20 or 10 modifications (e.g., substitutions, e.g., conservative substitutions) of an amino acid sequence of 157-160, 184-215, 478, 480, 483, and 485, or a sequence at least 95% identical to (e.g., with 95-99% identity with) an amino acid sequence of SEQ ID NO: 157-160, 184-215, 478, 480, 483, and 485.

In another embodiment, the encoded CD123 binding domain comprises a heavy chain variable region comprising an amino acid sequence selected from the group consisting of SEQ ID NO: 216-219 or 243-274, or an amino acid sequence having at least one, two or three modifications (e.g., substitutions, e.g., conservative substitutions) but not more than 30, 20 or 10 modifications (e.g., substitutions, e.g., conservative substitutions) of SEQ ID NO: 216-219 or 243-274, or a sequence at least 95% identical to (e.g., with 95-99% identity with) SEQ ID NO: 216-219 or 243-274. In another embodiment, the encoded CD123 binding domain comprises a heavy chain variable region comprising an amino acid sequence corresponding to the heavy chain variable region of SEQ ID NO:478, 480, 483, or 485, or an amino acid sequence having at least one, two or three modifications (e.g., substitutions, e.g., conservative substitutions) but not more than 30, 20 or 10 modifications (e.g., substitutions, e.g., conservative substitutions) of the corresponding portion of SEQ ID NO:478, 480, 483, or 485, or a sequence at least 95% identical to (e.g., with 95-99% identity with) to the corresponding portion of SEQ ID NO:478, 480, 483, or 485.

In another embodiment, the encoded CD123 binding domain comprises a light chain variable region comprising an amino acid sequence selected from the group consisting of SEQ ID NO: 275-278 or 302-333, or an amino acid sequence having at least one, two or three modifications (e.g., substitutions, e.g., conservative substitutions) but not more than 30, 20 or 10 modifications (e.g., substitutions, e.g., conservative substitutions) of SEQ ID NO: 275-278 or 302-333, or a sequence at least 95% identical to (e.g., with 95-99% identity with) SEQ ID NO: 275-278 or 302-333. In another embodiment, the encoded CD123 binding domain comprises a light chain variable region comprising an amino acid sequence corresponding to the light chain variable region of SEQ ID NO:478, 480, 483, or 485, or an amino acid sequence having at least one, two or three modifications (e.g., substitutions, e.g., conservative substitutions) but not more than 30, 20 or 10 modifications (e.g., substitutions, e.g., conservative substitutions) of the corresponding portion of SEQ ID NO:478, 480, 483, or 485, or a sequence at least 95% identical to (e.g., with 95-99% identity with) the corresponding portion of SEQ ID NO:478, 480, 483, or 485.

In one embodiment, the nucleic acid molecule encoding the scFv comprises a nucleotide sequence selected from the group consisting of SEQ ID NO: 479, 481, 482, 484, or a sequence with at least 95% identity, e.g., 95-99% identity thereof. In one embodiment, the nucleic acid molecule comprises a nucleotide sequence encoding the heavy chain variable region and/or the light chain variable region, wherein said nucleotide sequence comprises a portion of a nucleotide sequence selected from the group consisting of SEQ ID NO: 479, 481, 482, and 484, or a sequence with at least 95% identity, e.g., 95-99% identity thereof, corresponding to the heavy chain variable region and/or the light chain variable region. In one embodiment, the nucleic acid molecule comprises a nucleotide sequence encoding the heavy chain variable region and/or the light chain variable region, wherein the encoded amino acid sequence is selected from the group consisting of SEQ ID NO:157-160, or a sequence at least 95% identical (e.g., with 95-99% identity) thereof. In one embodiment, the nucleic acid molecule encodes an scFv comprising an amino acid sequence selected from the group consisting of SEQ ID NO:184-215, or a sequence with at least 95% identity, e.g., 95-99% identity thereof. In one embodiment, the nucleic acid molecule comprises a sequence encoding the heavy chain variable region and/or the light chain variable region, wherein the encoded amino acid sequence is selected from the group consisting of SEQ ID NO:184-215, or a sequence with at least 95% identity, e.g., 95-99% identity thereof.

In one embodiment, the encoded CD123 binding domain includes a (Gly4-Ser)n linker, wherein n is 1, 2, 3, 4, 5, or 6, preferably 3 or 4 (SEQ ID NO:26). The light chain variable region and heavy chain variable region of a scFv can be, e.g., in any of the following orientations: light chain variable region-linker-heavy chain variable region or heavy chain variable region-linker-light chain variable region.

### CD19 CAR Antigen Binding Domains

In embodiments, the CAR molecule is capable of binding CD19 (e.g., wild-type or mutant CD19). In embodiments, the CAR molecule comprises an anti-CD19 binding domain comprising one or more (e.g., all three) light chain complementary determining region 1 (LC CDR1), light chain complementary determining region 2 (LC CDR2), and light chain complementary determining region 3 (LC CDR3) of an anti-CD123 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of an anti-CD19 binding domain described herein, e.g., an anti-CD19 binding domain comprising one or more, e.g., all three, LC CDRs and one or more, e.g., all three, HC CDRs.

In one embodiment, the anti-CD19 binding domain comprises one or more (e.g., all three) heavy chain complementary determining region 1 (HC CDR1), heavy chain complementary determining region 2 (HC CDR2), and heavy chain complementary determining region 3 (HC CDR3) of an anti-CD19 binding domain described herein, e.g., the anti-CD19 binding domain has two variable heavy chain regions, each comprising a HC CDR1, a HC CDR2 and a HC CDR3 described herein. In one embodiment, the anti-CD19 binding domain comprises a murine light chain variable region described herein (e.g., in Table 14A) and/or a murine heavy chain variable region described herein (e.g., in Table 14A). In one embodiment, the anti-CD19 binding domain is a scFv comprising a murine light chain and a murine heavy chain of an amino acid sequence of Table 14A. In an embodiment, the anti-CD19 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 14A, or a sequence with at least 95% identity, e.g., 95-99% identity, with an amino acid sequence of Table 14A; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 14A, or a sequence with at least 95% identity, e.g., 95-99% identity, to an amino acid sequence of Table 14A. In one embodiment, the anti-CD19 binding domain comprises a sequence of SEQ ID NO: 774, or a sequence with at least 95% identity, e.g., 95-99% identity, thereof. In one embodiment, the anti-CD19 binding domain is a scFv, and a light chain variable region comprising an amino acid sequence described herein, e.g., in Table 14A, is attached to a heavy chain variable region comprising an amino acid sequence described herein, e.g., in Table 14A, via a linker, e.g., a linker described herein. In one embodiment, the anti-CD19 binding domain includes a (Gly₄-Ser)n linker, wherein n is 1, 2, 3, 4, 5, or 6, preferably 3 or 4 (SEQ ID NO: 26). The light chain variable region and heavy chain variable region of a scFv can be, e.g., in any of the following orientations: light chain variable region-linker-heavy chain variable region or heavy chain variable region-linker-light chain variable region.

In one embodiment, the CAR molecule comprises a humanized anti-CD19 binding domain that includes one or more (e.g., all three) light chain complementary determining region 1 (LC CDR1), light chain complementary determining region 2 (LC CDR2), and light chain complementary determining region 3 (LC CDR3) of a humanized anti-CD19 binding domain described herein, and one or more (e.g., all three) heavy chain complementary determining region 1 (HC CDR1), heavy chain complementary determining region 2 (HC CDR2), and heavy chain complementary determining region 3 (HC CDR3) of a humanized anti-CD19 binding domain described herein, e.g., a humanized anti-CD19 binding domain comprising one or more, e.g., all three, LC CDRs and one or more, e.g., all three, HC CDRs. In one embodiment, the humanized anti-CD19 binding domain comprises at least HC CDR2. In one embodiment, the humanized anti-CD19 binding domain comprises one or more (e.g., all three) heavy chain complementary determining region 1 (HC CDR1), heavy chain complementary determining region 2 (HC CDR2), and heavy chain complementary determining region 3 (HC CDR3) of a humanized anti-CD19 binding domain described herein, e.g., the humanized anti-CD19 binding domain has two variable heavy chain regions, each comprising a HC CDR1, a HC CDR2 and a HC CDR3 described herein. In one embodiment, the humanized anti-CD19 binding domain comprises at least HC CDR2. In one embodiment, the light chain variable region comprises one, two, three or all four framework regions of VK3_L25 germline sequence. In one embodiment, the light chain variable region has a modification (e.g., substitution, e.g., a substitution of one or more amino acid found in the corresponding position in the murine light chain variable region of SEQ ID NO: 773, e.g., a substitution at one or more of positions 71 and 87). In one embodiment, the heavy chain variable region comprises one, two, three or all four framework regions of VH4_4-59 germline sequence. In one embodiment, the heavy chain variable region has a modification (e.g., substitution, e.g., a substitution of one or more amino acid found in the corresponding position in the murine heavy chain variable region of SEQ ID NO: 773, e.g., a substitution at one or more of positions 71, 73 and 78). In one embodiment, the humanized anti-CD19 binding domain comprises a light chain variable region described herein (e.g., in Table 13A) and/or a heavy chain variable region described herein (e.g., in Table 13A). In one embodiment, the humanized anti-CD19 binding domain is a scFv comprising a light chain and a heavy chain of an amino acid sequence of Table 13A. In an embodiment, the humanized anti-CD19 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 13A, or a sequence with at least 95% identity, e.g., 95-99% identity, with an amino acid sequence of Table 13A; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 13A, or a sequence with at least 95% identity, e.g., 95-99% identity, to an amino acid sequence of Table 13A. In one embodiment, the humanized anti-CD19 binding domain comprises a sequence selected from the group consisting of SEQ ID NOs: 710-721, or a sequence with at least 95% identity, e.g., 95-99% identity, thereof. In one embodiment, the humanized anti-CD19 binding domain is a scFv, and a light chain variable region comprising an amino acid sequence described herein, e.g., in Table 13A, is attached to a heavy chain variable region comprising an amino acid sequence described herein, e.g., in Table 13A, via a linker, e.g., a linker described herein.

In embodiments, the antigen recognition domain binds CD19. In embodiments, the CAR comprises an amino acid sequence of a CD19 CAR described herein. In embodiments, the CAR comprises the amino acid sequence of SEQ ID NO: 773.

In one embodiment, the humanized anti-CD19 binding domain includes a (Gly₄-Ser)n linker, wherein n is 1, 2, 3, 4, 5, or 6, preferably 3 or 4 (SEQ ID NO: 26). The light chain variable region and heavy chain variable region of a scFv can be, e.g., in any of the following orientations: light chain variable region-linker-heavy chain variable region or heavy chain variable region-linker-light chain variable region.

### Other CAR Domains

In one embodiment, the CAR molecule comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO: 6. In one embodiment, the transmembrane domain comprises an amino acid sequence having at least one, two or three modifications (e.g., substitutions) but not more than 20, 10 or 5 modifications (e.g., substitutions) of an amino acid sequence of SEQ ID NO: 6, or a sequence with at least 95% identity, e.g., 95-99% identity, to an amino acid sequence of SEQ ID NO: 6.

In one embodiment, the antigen binding domain (e.g., CD123 or CD19 binding domain) is connected to the transmembrane domain by a hinge region, e.g., a hinge region described herein. In one embodiment, the encoded hinge region comprises SEQ ID NO:2, SEQ ID NO: 4, or SEQ ID NO:3, or a sequence with at least 95% identity, e.g., 95-99% identity, thereof.

In one embodiment, the CAR molecule further comprises a sequence encoding a costimulatory domain, e.g., a costimulatory domain described herein. In one embodiment, the costimulatory domain comprises a functional signaling domain of a protein selected from the group consisting of OX40, CD2, CD27, CD28, CDS, ICAM-1, LFA-1 (CD11a/CD18), ICOS, and 4-1BB (CD137). In one embodiment, the costimulatory domain comprises a sequence of SEQ ID NO: 7. In one embodiment, the costimulatory domain comprises a sequence of SEQ ID NO:8. In one embodiment, the costimulatory domain comprises a sequence of SEQ ID NO:43. In one embodiment, the costimulatory domain comprises a sequence of SEQ ID NO:45. In one embodiment, the costimulatory domain comprises an amino acid sequence having at least one, two or three modifications (e.g., substitutions) but not more than 20, 10 or 5 modifications (e.g., substitutions) of an amino acid sequence of SEQ ID NO: 7, 8, 43, or 45, or a sequence with at least 95% identity, e.g., 95-99% identity, to an amino acid sequence of SEQ ID NO: 7, 8, 43, or 45.

In one embodiment, the CAR molecule further comprises a sequence encoding an intracellular signaling domain, e.g., an intracellular signaling domain described herein. In one embodiment, the intracellular signaling domain comprises a functional signaling domain of 4-1BB and/or a functional signaling domain of CD3 zeta. In one embodiment, the intracellular signaling domain comprises the sequence of SEQ ID NO: 7 and/or the sequence of SEQ ID NO: 9 or 10. In one embodiment, the intracellular signaling domain comprises a functional signaling domain of CD27 and/or a functional signaling domain of CD3 zeta. In one embodiment, the intracellular signaling domain comprises the sequence of SEQ ID NO: 8 and/or the sequence of SEQ ID NO: 9 or 10. In one embodiment, the intracellular signaling domain comprises an amino acid sequence having at least one, two or three modifications (e.g., substitutions) but not more than 20, 10 or 5 modifications (e.g., substitutions) of an amino acid sequence of SEQ ID NO:7 or SEQ ID NO:8 and/or an amino acid sequence of SEQ ID NO:9 or SEQ ID NO:10, or a sequence with at least 95% identity, e.g., 95-99% identity, to an amino acid sequence of SEQ ID NO:7 or SEQ ID NO:8 and/or an amino acid sequence of SEQ ID NO:9 or SEQ ID NO:10. In one embodiment, the intracellular signaling domain comprises the sequence of SEQ ID NO: 7 or SEQ ID NO:8 and the sequence of SEQ ID NO: 9 or SEQ ID NO:10, wherein the sequences comprising the intracellular signaling domain are expressed in the same frame and as a single polypeptide chain.

In one embodiment, the CAR molecule further comprises a leader sequence, e.g., a leader sequence described herein. In one embodiment, the leader sequence comprises an amino acid sequence of SEQ ID NO: 1, or a sequence with at least 95% identity, e.g., 95-99% identity, to an amino acid sequence of SEQ ID NO:1.

### CD123 CAR Construct

In embodiments, the CAR molecule comprises a leader sequence, e.g., a leader sequence described herein, e.g., a leader sequence of SEQ ID NO: 1, or having at least 95% identity, e.g., 95-99% identity, thereof, a CD123 binding domain described herein, e.g., a CD123 binding domain comprising a LC CDR1, a LC CDR2, a LC CDR3, a HC CDR1, a HC CDR2 and a HC CDR3 described herein, e.g., a CD123 binding domain described in Table 11A or 12A, or a sequence with at least 95% identity, e.g., 95-99% identity, thereof, a hinge region, e.g., a hinge region described herein, e.g., a hinge region of SEQ ID NO:2, or having at least 95% identity, e.g., 95-99% identity, thereof, a transmembrane domain, e.g., a transmembrane domain described herein, e.g., a transmembrane domain having a sequence of SEQ ID NO:6 or a sequence having at least 95% identity, e.g., 95-99% identity, thereof, an intracellular signaling domain, e.g., an intracellular signaling domain described herein (e.g., an intracellular signaling domain comprising a costimulatory domain and/or a primary signaling domain). In one embodiment, the intracellular signaling domain comprises a costimulatory domain, e.g., a costimulatory domain described herein, e.g., a 4-1BB costimulatory domain having a sequence of SEQ ID NO:7, or having at least 95% identity, e.g., 95-99% identity, thereof, and/or a primary signaling domain, e.g., a primary signaling domain described herein, e.g., a CD3 zeta stimulatory domain having a sequence of SEQ ID NO:9 or SEQ ID NO:10, or having at least 95% identity, e.g., 95-99% identity, thereof. In one embodiment, the intracellular signaling domain comprises a costimulatory domain, e.g., a costimulatory domain described herein, e.g., a 4-1BB costimulatory domain having a sequence of SEQ ID NO:7, and/or a primary signaling domain, e.g., a primary signaling domain described herein, e.g., a CD3 zeta stimulatory domain having a sequence of SEQ ID NO:9 or SEQ ID NO:10.

### CD19 CAR Construct

In one embodiment, the CAR molecule comprises a leader sequence, e.g., a leader sequence described herein, e.g., a leader sequence of SEQ ID NO: 1, or having at least 95% identity, e.g., 95-99% identity, thereof; an anti-CD19 binding domain described herein, e.g., an anti-CD19 binding domain comprising a LC CDR1, a LC CDR2, a LC CDR3, a HC CDR1, a HC CDR2 and a HC CDR3 described herein, e.g., a murine anti-CD19 binding domain described in Table 14A, a humanized anti-CD19 binding domain described in Table 13A, or a sequence with 95-99% identify thereof; a hinge region, e.g., a hinge region described herein, e.g., a hinge region of SEQ ID NO: 2, 3, or 4, or having at least 95% identity, e.g., 95-99% identity, thereof; a transmembrane domain, e.g., a transmembrane domain described herein, e.g., a transmembrane domain having a sequence of SEQ ID NO:6 or a sequence having at least 95% identity, e.g., 95-99% identity, thereof; an intracellular signaling domain, e.g., an intracellular signaling domain described herein (e.g., an intracellular signaling domain comprising a costimulatory domain and/or a primary signaling domain). In one embodiment, the intracellular signaling domain comprises a costimulatory domain, e.g., a costimulatory domain described herein, e.g., a 4-1BB costimulatory domain having a sequence of SEQ ID NO:7, a CD28 costimulatory domain having a sequence of SEQ ID NO:43, a CD27 costimulatory domain having a sequence of SEQ ID NO: 8, or an ICOS costimulatory domain having a sequence of SEQ ID NO: 45, or having at least 95% identity, e.g., 95-99% identity, thereof, and/or a primary signaling domain, e.g., a primary signaling domain described herein, e.g., a CD3 zeta stimulatory domain having a sequence of SEQ ID NO:9 or SEQ ID NO:10, or having at least 95% identity, e.g., 95-99% identity, thereof.

### Other Exemplary CAR Constructs

In one embodiment, the CAR molecule comprises (e.g., consists of) an amino acid sequence described in US-2015-0283178-A1, US-2016-0046724-A1, US2014/0322212A1, US2016/0068601A1, US2016/0051651A1, US2016/0096892A1, US2014/0322275A1, or WO2015/090230; or an amino acid sequence having at least one, two, three, four, five, 10, 15, 20 or 30 modifications (e.g., substitutions) but not more than 60, 50 or 40 modifications (e.g., substitutions) of an amino acid sequence described in US-2015-0283178-A1, US-2016-0046724-A1, US2014/0322212A1, US2016/0068601A1, US2016/0051651A1, US2016/0096892A1, US2014/0322275A1, or WO2015/090230; or an amino acid sequence having 85%, 90%, 95%, 96%, 97%, 98% or 99% identity to an amino acid sequence described in US-2015-0283178-A1, US-2016-0046724-A1, US2014/0322212A1, US2016/0068601A1, US2016/0051651A1, US2016/0096892A1, US2014/0322275A1, or WO2015/090230.

### Vectors

In one embodiment, the cell expressing the CAR molecule comprises a vector that includes a nucleic acid sequence encoding the CAR molecule. In one embodiment, the vector is selected from the group consisting of a DNA, a RNA, a plasmid, a lentivirus vector, adenoviral vector, or a retrovirus vector. In one embodiment, the vector is a lentivirus vector. In one embodiment, the vector further comprises a promoter. In one embodiment, the promoter is an EF-1 promoter. In one embodiment, the EF-1 promoter comprises a sequence of SEQ ID NO: 11. In one embodiment, the vector is an in vitro transcribed vector, e.g., a vector that transcribes RNA of a nucleic acid molecule described herein. In one embodiment, the nucleic acid sequence in the in vitro vector further comprises a poly(A) tail, e.g., a poly A tail described herein, e.g., comprising about 150 adenosine bases (SEQ ID NO:30). In one embodiment, the nucleic acid sequence in the in vitro vector further comprises a 3′UTR, e.g., a 3′ UTR described herein, e.g., comprising at least one repeat of a 3′UTR derived from human beta-globulin. In one embodiment, the nucleic acid sequence in the in vitro vector further comprises promoter, e.g., a T2A promoter.

### CAR-Expressing Cells

In certain embodiments of the compositions and methods disclosed herein, the cell expressing the CAR molecule (also referred to herein as a “CAR-expressing cell”) is a cell or population of cells as described herein, e.g., a human immune effector cell or population of cells (e.g., a human T cell or a human NK cell, e.g., a human T cell described herein or a human NK cell described herein). In one embodiment, the human T cell is a CD8+ T cell. In one embodiment, the cell is an autologous T cell. In one embodiment, the cell is an allogeneic T cell. In one embodiment, the cell is a T cell and the T cell is diaglycerol kinase (DGK) deficient. In one embodiment, the cell is a T cell and the T cell is Ikaros deficient. In one embodiment, the cell is a T cell and the T cell is both DGK and Ikaros deficient. It shall be understood that the compositions and methods disclosed herein reciting the term “cell” encompass compositions and methods comprising one or more cells, e.g., a population of cells.

In some embodiments, the CAR-expressing cell that is administered comprises a regulatable CAR (RCAR), e.g., an RCAR as described herein. The RCAR may comprise, e.g., an intracellular signaling member comprising an intracellular signaling domain and a first switch domain, an antigen binding member comprising an antigen binding domain that binds an antigen (e.g., antigen described herein, e.g., B cell antigen, e.g., CD123 or CD19) and a second switch domain; and a transmembrane domain. The method may further comprise administering a dimerization molecule, e.g., in an amount sufficient to cause dimerization of the first switch and second switch domains.

### Inhibitors

In embodiments, the JAK-STAT inhibitor comprises/is an antibody molecule, a small molecule, a polypeptide, e.g., a fusion protein, or an inhibitory nucleic acid, e.g., a siRNA or shRNA. In embodiments, the JAK-STAT inhibitor is a small molecule, e.g., ruxolitinib, AG490, AZD1480, tofacitinib (tasocitinib or CP-690550), CYT387, fedratinib, baricitinib (INCB039110), lestaurtinib (CEP701), pacritinib (SB1518), XL019, gandotinib (LY2784544), BMS911543, fedratinib (SAR302503), decemotinib (V-509), INCB39110, GEN1, GEN2, GLPG0634, NS018, and N-(cyanomethyl)-4-[2-(4-morpholinoanilino)pyrimidin-4-yl]benzamide, or pharmaceutically acceptable salts thereof. In embodiments, the JAK-STAT inhibitor is ruxolitinib or a pharmaceutically acceptable salt thereof.

In embodiments, the BTK inhibitor comprises/is an antibody molecule, a small molecule, a polypeptide, e.g., a fusion protein, or an inhibitory nucleic acid, e.g., a siRNA or shRNA. In embodiments, the BTK inhibitor is a small molecule, e.g., ibrutinib, GDC-0834, RN-486, CGI-560, CGI-1764, HM-71224, CC-292, ONO-4059, CNX-774, or LFM-A13, or a pharmaceutically acceptable salt thereof, or a combination thereof. In embodiments, the BTK inhibitor is ibrutinib or a pharmaceutically acceptable salt thereof.

In embodiments, an IL-6 inhibitor, e.g., used in accordance with any composition or method described herein, comprises an inhibitor of IL-6 signaling, e.g., comprising an IL-6 inhibitor or an IL-6 receptor (IL-6R) inhibitor. Exemplary IL-6 inhibitors include tocilizumab, siltuximab, bazedoxifene, and soluble glycoprotein 130 (sgp130) blockers. Exemplary IL-6 inhibitors are described in International Application WO2014011984, which is hereby incorporated by reference. Tocilizumab is described in greater detail herein, e.g., in the “CRS Therapies” section herein. In one embodiment, the IL-6 inhibitor is an anti-IL-6 antibody, e.g., an anti-IL-6 chimeric monoclonal antibody such as siltuximab. In other embodiments, the inhibitor comprises a soluble gp130 or a fragment thereof that is capable of blocking IL-6 signalling. In some embodiments, the sgp130 or fragment thereof is fused to a heterologous domain, e.g., an Fc domain, e.g., is a gp130-Fc fusion protein such as FE301. In embodiments, the IL-6 inhibitor comprises an antibody, e.g., an antibody to the IL-6 receptor, such as sarilumab, olokizumab (CDP6038), elsilimomab, sirukumab (CNTO 136), ALD518/BMS-945429, ARGX-109, or FM101. In some embodiments, the IL-6 inhibitor comprises a small molecule such as CPSI-2364.

### Diseases

In embodiments, the disease associated with expression of an antigen is a hyperproliferative disorder, e.g., cancer. In embodiments, the cancer is a solid cancer. In other embodiments, the cancer is a hematological cancer.

In embodiments, the hematological cancer is a leukemia. In embodiments, the hematological cancer is acute myeloid leukemia (AML), acute lymphocytic leukemia (ALL), or chronic lymphocytic leukemia (CLL). In embodiments, the hematological cancer is a lymphoma, e.g., mantle cell lymphoma (MCL).

In embodiments, the hematological cancer is a B cell malignancy, e.g., B cell leukemia or B cell lymphoma.

In embodiments, the hematological cancer is chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia and myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, or unclassifiable lymphoma.

In embodiments, the hematological cancer is chosen from: acute myeloid leukemia (AML), acute lymphoblastic leukemia (ALL), acute lymphoblastic B-cell leukemia (B-cell acute lymphoid leukemia, BALL), acute lymphoblastic T-cell leukemia (T-cell acute lymphoid leukemia (TALL), B-cell prolymphocytic leukemia, chronic lymphocytic leukemia, chronic myeloid leukemia (CML), hairy cell leukemia, Hodgkin lymphoma, a histiocytic disorder, a mast cell disorder, a myelodysplasia, a myelodysplastic syndrome, a myeloproliferative neoplasm, a plasma cell myeloma, a plasmacytoid dendritic cell neoplasm, or a combination thereof.

In embodiments, the disease is a disease associated with expression of a B-cell antigen (e.g., expression of one or more of CD10, CD19, CD20, CD22, CD34, CD123, FLT-3, ROR1, CD79b, CD179b, and/or CD79a). In embodiments the disease associated with expression of a B-cell antigen is selected from a proliferative disease such as a cancer, a malignancy, or a precancerous condition such as a myelodysplasia, a myelodysplastic syndrome or a preleukemia, or is a non-cancer related indication associated with expression of the B-cell antigen, e.g., one or more of CD10, CD19, CD20, CD22, CD34, CD123, FLT-3, ROR1, CD79b, CD179b, and/or CD79a. In certain embodiments, the disease associated with B-cell antigen expression is a “preleukemia” which is a diverse collection of hematological conditions united by ineffective production (or dysplasia) of myeloid blood cells. In some embodiments, the disease associated with B-cell antigen expression includes, but is not limited to atypical and/or non-classical cancers, malignancies, precancerous conditions or proliferative diseases expressing the B-cell antigen (e.g., one or more of CD10, CD19, CD20, CD22, CD34, CD123, FLT-3, ROR1, CD79b, CD179b, and/or CD79a). In embodiments, the disease associated with expression of a B-cell antigen is a hematological cancer, leukemia, lymphoma, MCL, CLL, ALL, Hodgkin lymphoma, or multiple myeloma. Any combination of the diseases associated with B-cell antigen expression described herein can be treated with the methods and compositions described herein.

### CRS

In embodiments, the CRS is a severe CRS, e.g., grade 4 or 5 CRS. In embodiments, the CRS is a less than severe CRS, e.g., grade 1, 2, or 3 CRS. Additional description of CRS is provided in the section entitled “Cytokine Release Syndrome.”

In embodiments of any method described herein, the CRS is a CRS distinguished from sepsis, e.g., by a method described herein, e.g., by a method of distinguishing between CRS and sepsis in a subject as described herein. In embodiments, the method of distinguishing between CRS and sepsis comprises acquiring a measure of one or more of the following:

(i) the level or activity of one or more of (e.g., 2, 3, 4, 5, 6, 7, 8, 9, 10, 11, 12, 13, 14, 15, or all of) GM-CSF, HGF, IFN-γ, IFN-α, IL-10, IL-15, IL-5, IL-6, IL-8, IP-10, MCP1, MIG, MIP-1β, sIL-2Rα, sTNFRI, and sTNFRII, wherein a level or activity that is higher than a reference is indicative of CRS; or

(ii) the level or activity of one or more of (e.g., 2, 3, 4, 5, 6, or all of) CD163, IL-1β, sCD30, sIL-4R, sRAGE, sVEGFR-1, and sVEGFR-2, wherein a level or activity that is higher than a reference is indicative of sepsis. Additional embodiments of a method of distinguishing between CRS and sepsis in a subject are described herein.

### Dosing Regimens

In some embodiments, the CAR-expressing cell and the inhibitor (e.g., JAK-STAT or BTK inhibitor) are administered sequentially, concurrently, or within a treatment interval, e.g., as described herein.

In one embodiment, the CAR-expressing cell and the inhibitor (e.g., JAK-STAT or BTK inhibitor) are administered sequentially. In one embodiment, the inhibitor (e.g., JAK-STAT or BTK inhibitor) is administered prior to administration of the CAR-expressing cell. In one embodiment, the inhibitor (e.g., JAK-STAT or BTK inhibitor) is administered after the administration of the CAR-expressing cell.

In one embodiment, the inhibitor (e.g., JAK-STAT or BTK inhibitor) and CAR-expressing cell are administered simultaneously or concurrently.

In embodiments, the CAR-expressing cell and the inhibitor (e.g., JAK-STAT or BTK inhibitor) are administered in a treatment interval. In one embodiment, the treatment interval comprises a single dose of the inhibitor (e.g., JAK-STAT or BTK inhibitor) and a single dose of the CAR-expressing cell (e.g., in any order). In another embodiment, the treatment interval comprises multiple doses (e.g., a first and second dose) of the inhibitor (e.g., JAK-STAT or BTK inhibitor) and a dose of the CAR-expressing cell (e.g., in any order).

Where the treatment interval comprises a single dose of the inhibitor (e.g., JAK-STAT or BTK inhibitor) and a single dose of the CAR-expressing cell, in certain embodiments, the dose of inhibitor (e.g., JAK-STAT or BTK inhibitor) and the dose of the CAR-expressing cell are administered simultaneously or concurrently. For example, the dose of the inhibitor (e.g., JAK-STAT or BTK inhibitor) and the dose of the CAR-expressing cell are administered within 2 days (e.g., within 2 days, 1 day, 24 hours, 12 hours, 6 hours, 4 hours, 2 hours, 1 hour, or less) of each other. In embodiments, the treatment interval is initiated upon administration of the first-administered dose and completed upon administration of the later-administered dose.

Where the treatment interval comprises a single dose of the inhibitor (e.g., JAK-STAT or BTK inhibitor) and a single dose of the CAR-expressing cell, in certain embodiments, the dose of the inhibitor (e.g., JAK-STAT or BTK inhibitor) and the dose of the CAR-expressing cell are administered sequentially. In embodiments, the dose of the CAR-expressing cell is administered prior to the dose of the inhibitor (e.g., JAK-STAT or BTK inhibitor), and the treatment interval is initiated upon administration of the dose of the CAR-expressing cell and completed upon administration of the dose of the inhibitor (e.g., JAK-STAT or BTK inhibitor). In other embodiments, the dose of the inhibitor (e.g., JAK-STAT or BTK inhibitor) is administered prior to the dose of the CAR-expressing cell, and the treatment interval is initiated upon administration of the dose of the inhibitor (e.g., JAK-STAT or BTK inhibitor) and completed upon administration of the dose of the CAR-expressing cell. In one embodiment, the treatment interval further comprises one or more, e.g., 1, 2, 3, 4, 5, 6, 7, 8, 9, 10, 11, 12, 13, 14, 15, 16, 17, 18, 19, 20, or more, subsequent doses of the inhibitor (e.g., JAK-STAT or BTK inhibitor). In such embodiments, the treatment interval comprises two, three, four, five, six, seven, eight, nine, ten, or more, doses of inhibitor (e.g., JAK-STAT or BTK inhibitor) and one dose of the CAR-expressing cell. In one embodiment, the dose of the CAR-expressing cell is administered at least 1 day, 2 days, 3 days, 4 days, 5 days, 6 days, 7 days, or 2 weeks before or after a dose of inhibitor (e.g., JAK-STAT or BTK inhibitor) is administered. In embodiments where more than one dose of inhibitor (e.g., JAK-STAT or BTK inhibitor) is administered, the dose of the CAR-expressing cell is administered at least 1 day, 2 days, 3 days, 4 days, 5, days, 6 days, 7 days, or 2 weeks before or after the first dose of inhibitor (e.g., JAK-STAT or BTK inhibitor) is administered or after the initiation of the treatment interval. In embodiments, where more than one dose of inhibitor (e.g., JAK-STAT or BTK inhibitor) is administered, the second inhibitor (e.g., JAK-STAT or BTK inhibitor) dose is administered about 10 h, 12 h, 14 h, 16 h, 18 h, 20 h, 24 h, 1 day, 1.5 days, 2 days, 3 days, or 4 days after the first dose of inhibitor (e.g., JAK-STAT or BTK inhibitor) is administered.

Where the treatment interval comprises multiple doses (e.g., a first and second, and optionally a subsequent dose) of an inhibitor (e.g., JAK-STAT or BTK inhibitor) and a dose of a CAR-expressing cell, in certain embodiments, the dose of the CAR-expressing cell and the first dose of the inhibitor (e.g., JAK-STAT or BTK inhibitor) are administered simultaneously or concurrently, e.g., within 2 days (e.g., within 2 days, 1 day, 24 hours, 12 hours, 6 hours, 4 hours, 2 hours, or less) of each other. In embodiments, the second dose of the inhibitor (e.g., JAK-STAT or BTK inhibitor) is administered after either (i) the dose of the CAR-expressing cell or (ii) the first dose of the inhibitor (e.g., JAK-STAT or BTK inhibitor), whichever is later. In embodiments, the second dose of the inhibitor (e.g., JAK-STAT or BTK inhibitor) is administered at least 8 h (e.g., at least 8 h, 9 h, 10 h, 12 h, 14 h, 16 h, 18 h, 20 h, 24 h, 1 day, 1.5 days, 2 days, 3 days, 4 days, 5 days, 6 days, 7 days, 1 week, 2 weeks, 3 weeks, 4 weeks, 5 weeks, or more) after (i) or (ii). In embodiments, a subsequent dose (e.g., third, fourth, or fifth dose, and so on) of the inhibitor (e.g., JAK-STAT or BTK inhibitor) is administered after the second dose of the inhibitor (e.g., JAK-STAT or BTK inhibitor). In embodiments, the subsequent dose of the inhibitor (e.g., JAK-STAT or BTK inhibitor) is administered at least 8 h (e.g., at least 8 h, 9 h, 10 h, 12 h, 14 h, 16 h, 18 h, 20 h, 24 h, 1 day, 1.5 days, 2 days, 3 days, 4 days, 5 days, 6 days, 7 days, 1 week, 2 weeks, 3 weeks, 4 weeks, 5 weeks, or more) after the second dose of the inhibitor (e.g., JAK-STAT or BTK inhibitor). In such embodiments, the treatment interval is initiated upon administration of the first-administered dose and completed upon administration of the second dose (or subsequent dose) of the inhibitor (e.g., JAK-STAT or BTK inhibitor). In embodiments, the dose of inhibitor (e.g., JAK-STAT or BTK inhibitor) is administered once a day (QD) or twice a day (BID) for a treatment interval of at least 7 days, 8 days, 9 days, 10 days, 1 week, 2 weeks, 3 weeks, 4 weeks, 5 weeks, 6 weeks, 1 month, 2 months, 3 months, 4 months, 5 months, 6 months, 7 months, 8 months, or more. Any of the treatment intervals described herein can include one or more doses of the CAR-expressing cells.

In other embodiments where the treatment interval comprises multiple doses (e.g., a first and second, and optionally a subsequent dose) of an inhibitor (e.g., JAK-STAT or BTK inhibitor) and a dose of a CAR-expressing cell, the dose of the CAR-expressing cell and the first dose of the inhibitor (e.g., JAK-STAT or BTK inhibitor) are administered sequentially. In embodiments, the dose of the CAR-expressing cell is administered after administration of the first dose of the inhibitor (e.g., JAK-STAT or BTK inhibitor) but before the administration of the second dose of the inhibitor (e.g., JAK-STAT or BTK inhibitor). In embodiments, a subsequent dose (e.g., third, fourth, or fifth dose, and so on) of the inhibitor (e.g., JAK-STAT or BTK inhibitor) is administered after the second dose of the inhibitor (e.g., JAK-STAT or BTK inhibitor). In such embodiments, the treatment interval is initiated upon administration of the first dose of the inhibitor (e.g., JAK-STAT or BTK inhibitor) and completed upon administration of the second, third, fourth, fifth, or sixth dose (or subsequent dose) of the inhibitor (e.g., JAK-STAT or BTK inhibitor). In one embodiment, the second dose of the inhibitor (e.g., JAK-STAT or BTK inhibitor) is administered at least 8 h (e.g., at least 8 h, 9 h, 10 h, 12 h, 14 h, 16 h, 18 h, 20 h, 24 h, 1 day, 1.5 days, 2 days, 3 days, 4 days, 5 days, 6 days, 7 days, 1 week, 2 weeks, 3 weeks, 4 weeks, 5 weeks, or more) after administration of the first dose of the inhibitor (e.g., JAK-STAT or BTK inhibitor). In one embodiment, the subsequent dose (e.g., third, fourth, or fifth dose, and so on) of the inhibitor (e.g., JAK-STAT or BTK inhibitor) is administered at least 8 h (e.g., at least 8 h, 9 h, 10 h, 12 h, 14 h, 16 h, 18 h, 20 h, 24 h, 1 day, 1.5 days, 2 days, 3 days, 4 days, 5 days, 6 days, 7 days, 1 week, 2 weeks, 3 weeks, 4 weeks, 5 weeks, or more) after the second dose of the inhibitor (e.g., JAK-STAT or BTK inhibitor). In one embodiment, the dose of the CAR-expressing cell is administered at least 1 day (e.g., at least 1 day, 2 days, 3 days, 4 days, 5 days, 6 days, 7 days, 1 week, 2 weeks, 3 weeks, 4 weeks, 5 weeks, 6 weeks, 1 month, 2 months, 3 months, 4 months, 5 months, 6 months, or more) after administration of the first dose of the inhibitor (e.g., JAK-STAT or BTK inhibitor). In one embodiment, the second dose of the inhibitor (e.g., JAK-STAT or BTK inhibitor) is administered within 1 day (e.g., within 24 h, 20 h, 18 h, 16 h, 14 h, 12 h, 10 h, 8 h, 6 h, or less) of the administration of the dose of the CAR-expressing cell. In embodiments, the second dose of the inhibitor (e.g., JAK-STAT or BTK inhibitor) is administered concurrently with the dose of the CAR-expressing cell. In one embodiment, the second dose of the inhibitor (e.g., JAK-STAT or BTK inhibitor) is administered at least 1 day (e.g., at least 1 day, 2 days, 3 days, 4 days, 5 days, 6 days, 7 days, 1 week, 2 weeks, 3 weeks, 4 weeks, 5 weeks, or more) after administration of the dose of the CAR-expressing cell. In embodiments, the treatment interval comprises continuous dosing of the inhibitor (e.g., JAK-STAT or BTK inhibitor), e.g., once a day, twice a day, three times a day, every 2 days, every 3 days, or every 4 days. In embodiments where the inhibitor is dosed continuously, the dose (e.g., first dose) of the CAR-expressing cell is administered after the first dose of the inhibitor, e.g., at least 1 day after, e.g., at least 1, 2, 3, 4, 5, 6, 7 days, 1, 2, 3, 4, 5, 6 weeks, 1, 2, 3, 4, 5, 6 months or more after. In other embodiments where the inhibitor is dosed continuously, the dose (e.g., first dose) of the CAR-expressing cell is administered concurrently with (e.g., within 1 day (e.g., within 24 h, 20 h, 18 h, 16 h, 14 h, 12 h, 10 h, 8 h, 6 h, or less, or) the administration of the first dose of the inhibitor. In embodiments where the inhibitor is dosed continuously, the inhibitor is dosed for at least 1 day after, e.g., at least 1, 2, 3, 4, 5, 6, 7 days, 1, 2, 3, 4, 5, 6 weeks, 1, 2, 3, 4, 5, 6 months or more after, the administration of the first dose of the CAR-expressing cell. In other embodiments, the dose of the CAR-expressing cell is administered before administration of the first dose of the inhibitor (e.g., JAK-STAT or BTK inhibitor). In such embodiments, the treatment interval is initiated upon administration of the CAR-expressing cell and completed upon administration of the second dose (or subsequent dose) of the inhibitor (e.g., JAK-STAT or BTK inhibitor). In embodiments, the second dose of the inhibitor (e.g., JAK-STAT or BTK inhibitor) is administered at least 8 h (e.g., at least 8 h, 9 h, 10 h, 12 h, 14 h, 16 h, 18 h, 20 h, 24 h, 1 day, 1.5 days, 2 days, 3 days, 4 days, 5 days, 6 days, 7 days, 1 week, 2 weeks, 3 weeks, 4 weeks, 5 weeks, or more) after administration of the first dose of the inhibitor (e.g., JAK-STAT or BTK inhibitor). In embodiments, the subsequent dose (e.g., third, fourth, or fifth dose, and so on) of the inhibitor (e.g., JAK-STAT or BTK inhibitor) is administered at least 8 h (e.g., at least 8 h, 9 h, 10 h, 12 h, 14 h, 16 h, 18 h, 20 h, 24 h, 1 day, 1.5 days, 2 days, 3 days, 4 days, 5 days, 6 days, 7 days, 1 week, 2 weeks, 3 weeks, 4 weeks, 5 weeks, or more) after the second dose of the inhibitor (e.g., JAK-STAT or BTK inhibitor). In embodiments, the first dose of the inhibitor (e.g., JAK-STAT or BTK inhibitor) is administered at least 1 day (e.g., at least 1 day, 2 days, 3 days, 4 days, 5 days, 6 days, 7 days, 1 week, 2 weeks, 3 weeks, 4 weeks, 5 weeks, or more) after administration of the CAR-expressing cell. In embodiments, the dose of inhibitor (e.g., JAK-STAT or BTK inhibitor) is administered once a day (QD) or twice a day (BID) for a treatment interval of at least 7 days, 8 days, 9 days, 10 days, 1 week, 2 weeks, 3 weeks, 4 weeks, 5 weeks, 6 weeks, 1 month, 2 months, 3 months, 4 months, 5 months, 6 months, 7 months, 8 months, or more.

In one embodiment, any of the treatment intervals described herein can be repeated one or more times, e.g., 1, 2, 3, 4, or 5 more times. In one embodiment, the treatment interval is repeated once, resulting in a treatment regimen comprising two treatment intervals. In an embodiment, the repeated treatment interval is administered at least 1 day, e.g., 1 day, 2 days, 3 days, 4 days, 5 days, 6 days, 7 days, or 2 weeks, or more after the completion of the first or previous treatment interval. In an embodiment, the repeated treatment interval is administered at least 3 days after the completion of the first or previous treatment interval.

In one embodiment, any of the treatment intervals described herein can be followed by one or more, e.g., 1, 2, 3, 4, or 5, subsequent treatment intervals. The one or more subsequent treatment interval is different from the first or previous treatment interval. By way of example, a first treatment interval consisting of a single dose of an inhibitor (e.g., JAK-STAT or BTK inhibitor) and a single dose of a CAR-expressing cell is followed by a second treatment interval consisting of multiple doses (e.g., two, three, four, or more doses) of an inhibitor (e.g., JAK-STAT or BTK inhibitor) and a single dose of a CAR-expressing cell. In one embodiment, the one or more subsequent treatment intervals is administered at least 1 day, e.g., 1 day, 2 days, 3 days, 4 days, 5 days, 6 days, 7 days, or 2 weeks, after the completion of the first or previous treatment interval.

In any of the methods described herein, one or more subsequent doses, e.g., 1, 2, 3, 4, 5, 6, 7, 8, 9, 10, more doses, of the inhibitor (e.g., JAK-STAT or BTK inhibitor) is administered after the completion of one or more treatment intervals. In embodiments where the treatment intervals are repeated or two or more treatment intervals are administered, one or more subsequent doses, e.g., 1, 2, 3, 4, 5, 6, 7, 8, 9, 10, more doses, of the inhibitor (e.g., JAK-STAT or BTK inhibitor) is administered after the completion of one treatment interval and before the initiation of another treatment interval. In one embodiment, a dose of the inhibitor (e.g., JAK-STAT or BTK inhibitor) is administered every 8 h, 10 h, 12 h, 14 h, 16 h, 20 h, 24 h, 1 day, 1.5 days, 2 days 3 days, 4 days, 5 days, 7 days, 2 weeks, 3 weeks, or 4 weeks after the completion of one or more, or each, treatment intervals. In one embodiment, one, two, or three doses of the inhibitor (e.g., JAK-STAT or BTK inhibitor) is administered each day after the completion of one or more, or each, treatment intervals.

In any of the methods described herein, one or more, e.g., 1, 2, 3, 4, 5, or more, subsequent doses of the CAR-expressing cell are administered after the completion of one or more treatment intervals. In embodiments where the treatment intervals are repeated or two or more treatment intervals are administered, one or more subsequent doses, e.g., 1, 2, 3, 4, or 5, or more doses, of the CAR-expressing cell is administered after the completion of one treatment interval and before the initiation of another treatment interval. In one embodiment, a dose of the CAR-expressing cell is administered every 2 days, 3 days, 4 days, 5 days, 7 days, 2 weeks, 3 weeks, or 4 weeks after the completion of one or more, or each, treatment intervals.

In one embodiment, the treatment interval comprises a single dose of a CAR-expressing cell (e.g., a CD123 CAR-expressing cell or CD19 CAR-expressing cell) that is administered concurrently with (e.g., within 2 days (e.g., within 2 days, 1 day, 24 hours, 12 hours, 6 hours, 4 hours, 2 hours, or less, of) a first dose of an inhibitor (e.g., JAK-STAT inhibitor, e.g., ruxolitinib; or BTK inhibitor, e.g., ibrutinib). In embodiments, the JAK-STAT inhibitor (e.g., ruxolitinib) or the BTK inhibitor (e.g., ibrutinib) is administered twice a day (BID) during the duration of the treatment interval. In embodiments, the JAK-STAT inhibitor (e.g., ruxolitinib) or the BTK inhibitor (e.g., ibrutinib) is administered once a day (QD) during the duration of the treatment interval.

In other embodiments, the treatment interval comprises a single dose of a CAR-expressing cell (e.g., a CD123 CAR-expressing cell or CD19 CAR-expressing cell) that is administered after (e.g., 1 day, 2 days, 3 days, 4 days, 5 days, 6 days, 7 days, 1 week, 2 weeks, 3 weeks, 4 weeks, or more after) administration of a first dose of an inhibitor (e.g., JAK-STAT inhibitor, e.g., ruxolitinib; or BTK inhibitor, e.g., ibrutinib). In embodiments, a second dose of the inhibitor (e.g., JAK-STAT inhibitor, e.g., ruxolitinib; or BTK inhibitor, e.g., ibrutinib) is administered after administration of the first dose of the inhibitor (e.g., JAK-STAT inhibitor, e.g., ruxolitinib; or BTK inhibitor, e.g., ibrutinib). In embodiments, a subsequent dose of the inhibitor (e.g., JAK-STAT inhibitor, e.g., ruxolitinib; or BTK inhibitor, e.g., ibrutinib) is administered. In embodiments, the doses of the inhibitor (e.g., JAK-STAT inhibitor, e.g., ruxolitinib; or BTK inhibitor, e.g., ibrutinib) are administered twice a day (BID). In embodiments, the doses of the inhibitor (e.g., JAK-STAT inhibitor, e.g., ruxolitinib; or BTK inhibitor, e.g., ibrutinib) are administered once a day (QD). In embodiments, the treatment interval comprises at least 5 (e.g., at least 5, 6, 7, 8, 9, 10, 12, 14, 16, 18, 20, or more) doses of the inhibitor (e.g., JAK-STAT inhibitor, e.g., ruxolitinib; or BTK inhibitor, e.g., ibrutinib). In embodiments, the treatment interval comprises continuous dosing of the inhibitor (e.g., QD or BID). In embodiments, the treatment interval is for a duration of 1-7 days, 1-5 weeks, or 1-12 months.

In any of the methods described herein, the subject is administered a single dose of a CAR-expressing cell and a single dose of an inhibitor (e.g., JAK-STAT inhibitor, e.g., ruxolitinib; or BTK inhibitor, e.g., ibrutinib). In one embodiment, the single dose of the CAR-expressing cell is administered at least 1 day, e.g., 1, 2, 3, 4, 5, 6, 7, 8, 9, 10, 14, 20, 25, 30, 35, 40 days, or 2 weeks, 3 weeks, 4 weeks, or more, after administration of the single dose of the inhibitor (e.g., JAK-STAT inhibitor, e.g., ruxolitinib; or BTK inhibitor, e.g., ibrutinib).

In one embodiment, one or more, e.g., 1, 2, 3, 4, or 5, subsequent doses of a CAR-expressing cell are administered to the subject after the initial dose of the CAR-expressing cell. In one embodiment, the one or more subsequent doses of the CAR-expressing cell are administered at least 2 days, e.g., 2, 3, 4, 5, 6, 7, 8, 9, 10, 14, 20, 25, 30, 35, 40 days, or 2 weeks, 3 weeks, 4 weeks, or more, after the previous dose of the CAR-expressing cell. In one embodiment, the one or more subsequent doses of the CAR-expressing cell are administered at least 5 days after the previous dose of the CAR-expressing cell. In one embodiment, the subject is administered three doses of the CAR-expressing cell per week or one dose every 2 days.

In one embodiment, one or more, e.g., 1, 2, 3, 4, 5, 6, 7, 8, 9, 10, or more, subsequent doses of the inhibitor (e.g., JAK-STAT inhibitor, e.g., ruxolitinib; or BTK inhibitor, e.g., ibrutinib) are administered after administration of the single dose of the inhibitor (e.g., JAK-STAT inhibitor, e.g., ruxolitinib; or BTK inhibitor, e.g., ibrutinib). In one embodiment, the one or more subsequent doses of the inhibitor (e.g., JAK-STAT inhibitor, e.g., ruxolitinib; or BTK inhibitor, e.g., ibrutinib) are administered at least 5 days, 7 days, 10 days, 14 days, 20 days, 25 days, 30 days, 2 weeks, 3 weeks, 4 weeks, or 5 weeks, after the previous dose of inhibitor (e.g., JAK-STAT inhibitor, e.g., ruxolitinib; or BTK inhibitor, e.g., ibrutinib). In other embodiments, the one or more subsequent doses of the inhibitor (e.g., JAK-STAT inhibitor, e.g., ruxolitinib; or BTK inhibitor, e.g., ibrutinib) are administered every other day, once a day, or twice a day, after the previous dose of inhibitor (e.g., JAK-STAT inhibitor, e.g., ruxolitinib; or BTK inhibitor, e.g., ibrutinib).

In one embodiment, the one or more subsequent doses of the inhibitor (e.g., JAK-STAT inhibitor, e.g., ruxolitinib; or BTK inhibitor, e.g., ibrutinib) are administered at least 1, 2, 3, 4, 5, 6, or 7 days, after a dose of the CAR-expressing cell, e.g., the initial dose of the CAR-expressing cell.

In one embodiment, one or more, e.g., 1, 2, 3, 4, or 5, doses of the inhibitor (e.g., JAK-STAT inhibitor, e.g., ruxolitinib; or BTK inhibitor, e.g., ibrutinib) is administered prior to the first dose of the CAR-expressing cell.

In one embodiment, the administration of the one or more doses of the CAR-expressing cell and the one or more doses of inhibitor (e.g., JAK-STAT inhibitor, e.g., ruxolitinib; or BTK inhibitor, e.g., ibrutinib) is repeated, e.g., 1, 2, 3, 4, or 5 more times.

Dosages and therapeutic regimens of the therapeutic agents disclosed herein can be determined by a skilled artisan.

In any of the administration regimens or treatment intervals described herein, in some embodiments, a dose of CAR-expressing cells (e.g., CD19 CAR-expressing or CD123 CAR-expressing cells) comprises at least about 1×10⁵, 5×10⁶, 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells. In some embodiments, a dose of CAR-expressing cells comprises at least about 1-5×10⁷ to 1-5×10⁸. In some embodiments, the subject is administered about 1-5×10⁷ CAR-expressing cells. In other embodiments, the subject is administered about 1-5×10⁸ CAR-expressing cells.

In embodiments, the CAR-expressing cell is administered at a dose (e.g., total dose) of 1.5×10⁷ to 5×10⁹ cells per kg (e.g., 0.3×10⁶ to 1×10⁸ cells per kg). In embodiments, the total dose does not exceed 1.5×10¹⁰ cells/kg, e.g., administered over time in multiple doses, e.g., does not exceed 1.5×10⁹ cells/kg, e.g., does not exceed 1.5×10⁸ cells/kg.

In one embodiment, up to 10, 9, 8, 7, 6, 5, 4, 3, or 2 doses of cells are administered. In other embodiments, one, two, three, four, five or 6 doses of the cells are administered to the mammal, e.g., in a treatment interval of one, two, three, four or more weeks. In one embodiment, up to 6 doses are administered in two weeks. The doses may the same or different. In one embodiment, a lower dose is administered initially, followed by one or more higher doses. In one exemplary embodiment, the lower dose is about 1×10⁵ to 1×10⁹ cells/kg, or 1×10⁶ to 1×10⁸ cells/kg; and the higher dose is about 2×10⁵ to 2×10⁹ cells/kg or 2×10⁶ to 2×10⁸ cells/kg, followed by 3-6 doses of about 4×10⁵ to 4×10⁹ cells/kg, or 4×10⁶ to 4×10⁸ cells/kg.

In embodiments, the CAR-expressing cells are administered to the subject according to a dosing regimen comprising a total dose of cells administered to the subject by dose fractionation, e.g., one, two, three or more separate administration of a partial dose. In embodiments, a first percentage of the total dose is administered on a first day of treatment, a second percentage of the total dose is administered on a subsequent (e.g., second, third, fourth, fifth, sixth, or seventh or later) day of treatment, and optionally, a third percentage (e.g., the remaining percentage) of the total dose is administered on a yet subsequent (e.g., third, fourth, fifth, sixth, seventh, eighth, ninth, tenth, or later) day of treatment. For example, 10% of the total dose of cells is delivered on the first day, 30% of the total dose of cells is delivered on the second day, and the remaining 60% of the total dose of cells is delivered on the third day of treatment. For example, a total cell dose includes 1 to 5×10⁷ or 1 to 5×10⁸ CAR-expressing cells.

In embodiments, the total dose is administered over multiple doses (e.g., a first dose, a second dose, and optionally a third dose, and so on).

In embodiments, the first dose comprises about 10% of the total dose (e.g., about 1×10⁷ cells/kg), e.g., administered on a first day. In embodiments, the second dose comprises about 30% of the total dose (e.g., about 3×10⁷ cells/kg), e.g., administered on a subsequent days (e.g., 1, 2, 3, 4, 5, 6, or 7 days after the first dose). In embodiments, the second dose is administered if the subject is clinically stable after the first dose. In embodiments, a subsequent dose (e.g., third, optionally fourth, etc. dose) is administered to the subject, e.g., where the sum of the first dose, second dose, and subsequent dose add up to the total dose. In embodiments, where the total dose is administered over multiple doses, the time between each dose is at least 1 day (e.g., at least 1, 2, 3, 4, 5, 6, 7 days, 1, 2, or 3 weeks, or more). In embodiments, the time between the second dose and the third dose, and/or between the third dose and the fourth dose, and/or between the fourth dose and the fifth dose, is at least 1 week (e.g., at least 1, 2, 3, 4 weeks, or more).

In embodiments, in any of the administration regimens described herein, the dose of the inhibitor (e.g., JAK-STAT inhibitor or BTK inhibitor) is administered every 1, 2, 3, 4, 5, 6, or 7 days, or twice a day, or three times a day.

In embodiments, a JAK-STAT inhibitor, e.g., ruxolitinib, is administered (e.g., orally) at a dose of 2.5 mg to 50 mg (e.g., 2.5-5 mg, 5-10 mg, 10-15 mg, 15-20 mg, 20-25 mg, 25-30 mg, 30-35 mg, 35-40 mg, 40-45 mg, or 45-50 mg) twice daily (e.g., 5 mg to 100 mg total per day).

In embodiments, a BTK inhibitor, e.g., ibrutinib (PCI-32765), is administered (e.g., orally) at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle, or daily for 28 day cycle. In one embodiment, 1, 2, 3, 4, 5, 6, 7, 8, 9, 10, 11, 12 or more cycles of the BTK inhibitor, e.g., ibrutinib, are administered.

In some embodiments of any of the methods disclosed herein, the method comprises administering the inhibitor (e.g., BTK inhibitor, e.g., ibrutinib; or JAK-STAT inhibitor, e.g., ruxolitinib) to the subject, reducing the amount (e.g., ceasing administration) of the inhibitor, and subsequently administering the CAR-expressing cell (e.g., a CAR19- or CAR123-expressing cell) to the subject.

In some embodiments, the method comprises administering the inhibitor (e.g., BTK inhibitor, e.g., ibrutinib; or JAK-STAT inhibitor, e.g., ruxolitinib) to the subject and subsequently administering a combination of the inhibitor and the CAR-expressing cell (e.g., a CAR19- or CAR123-expressing cell) to the subject.

In some embodiments, the method comprises administering the inhibitor (e.g., BTK inhibitor, e.g., ibrutinib, or JAK-STAT inhibitor, e.g., ruxolitinib) to the subject, reducing the amount (e.g., ceasing or discontinuing administration) of the inhibitor, and subsequently administering a combination of the CAR-expressing cell (e.g., a CAR19- or CAR123-expressing cell) and a second inhibitor (e.g., a second inhibitor other than the first inhibitor) to the subject. In some embodiments, the first inhibitor is a BTK inhibitor and the second inhibitor is a BTK inhibitor other than the first BTK inhibitor, e.g., other than ibrutinib. In some embodiments, the first inhibitor is a JAK-STAT inhibitor and the second inhibitor is a JAK-STAT inhibitor other than the first JAK-STAT inhibitor, e.g., other than ruxolitinib. In some embodiments, the first inhibitor is a JAK-STAT inhibitor and the second inhibitor is a BTK inhibitor. In some embodiments, the first inhibitor is a BTK inhibitor and the second inhibitor is a JAK-STAT inhibitor. In some embodiments, the second BTK inhibitor is chosen from one or more of GDC-0834, RN-486, CGI-560, CGI-1764, HM-71224, CC-292, ONO-4059, CNX-774, or LFM-A13, or a combination thereof. In embodiments, the second JAK-STAT inhibitor is chosen from one or more of AG490, AZD1480, tofacitinib (tasocitinib or CP-690550), or CYT387.

In one embodiment, the cells expressing a CAR molecule, e.g., a CAR molecule described herein, are administered at a dose and/or dosing schedule described herein.

In an embodiment, any method described herein further comprises administering a therapy to prevent or treat CRS. In embodiments, the therapy comprises an IL-6 inhibitor (e.g., an anti-IL6 receptor inhibitor, e.g., an anti-IL6 receptor inhibitor, e.g., tocilizumab). In other embodiments, the therapy comprises an IL-6 inhibitor in combination with one or more (or all) of a vasoactive medication, an immunosuppressive agent, a corticosteroid, or mechanical ventilation. In embodiments, the method comprises administering the IL-6 inhibitor (e.g., tocilizumab) prior to (e.g., at least 1, 2, 3, 4, 5, 6, 7, 8, 9 or 10 days or 1, 2, 3, or 4 weeks prior to) administration of a dose (e.g., a first dose) of a CAR-expressing cell (e.g., CAR-expressing cell described herein). In embodiments, the method comprises administering the IL-6 inhibitor (e.g., tocilizumab) concurrently with administration of a dose (e.g., a first dose) of a CAR-expressing cell (e.g., CAR-expressing cell described herein). In embodiments, the method comprises administering the IL-6 inhibitor (e.g., tocilizumab) after the administration of a dose (e.g., a first dose) of a CAR-expressing cell (e.g., CAR-expressing cell described herein), e.g., but prior to or within 1 week (e.g., within 1 week, 7, 6, 5, 4, 3, 2, 1 day or less) of a first sign of a fever in the subject. In embodiments, the method comprises administering the IL-6 inhibitor (e.g., tocilizumab) after the administration of a dose (e.g., a first dose) of a CAR-expressing cell (e.g., CAR-expressing cell described herein), and within 1 week (e.g., within 1 week, 7, 6, 5, 4, 3, 2, 1 day or less) of the development of a temperature of at least 38° C. (e.g., at least 38.5° C.) in the subject, e.g., for two successive measurements in 24 hours (e.g., at least 4 hours apart). In embodiments, the subject has (e.g., is diagnosed with or identified as having) a high tumor burden prior to treatment with the CAR-expressing cell. In embodiments, a high tumor burden comprises at least 40% blasts (e.g., at least 40%, 45%, 50%, 60%, 70%, 80%, 90%, 95%, or more, blasts) in bone marrow of the subject prior to administration of the CAR-expressing cell (e.g., about 1-5 days prior to administration of the CAR-expressing cell).

In embodiments, the method comprises administering a dose of tocilizumab of about 5-15 mg/kg, e.g., 8-12 mg/kg (e.g., about 8 mg/kg, about 9 mg/kg, about 10 mg/kg, about 11 mg/kg, or about 12 mg/kg).

In one embodiment, the CAR molecule is introduced into T cells, e.g., using in vitro transcription, and the subject (e.g., human) receives an initial administration of cells comprising a CAR molecule, and one or more subsequent administrations of cells comprising a CAR molecule, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of cells comprising a CAR molecule are administered to the subject (e.g., human) per week, e.g., 2, 3, or 4 administrations of cells comprising a CAR molecule are administered per week. In one embodiment, the subject (e.g., human subject) receives more than one administration of cells comprising a CAR molecule per week (e.g., 2, 3 or 4 administrations per week) (also referred to herein as a cycle), followed by a week of no administration of cells comprising a CAR molecule, and then one or more additional administration of cells comprising a CAR molecule (e.g., more than one administration of the cells comprising a CAR molecule per week) is administered to the subject. In another embodiment, the subject (e.g., human subject) receives more than one cycle of cells comprising a CAR molecule, and the time between each cycle is less than 10, 9, 8, 7, 6, 5, 4, or 3 days. In one embodiment, the cells comprising a CAR molecule are administered every other day for 3 administrations per week. In one embodiment, the cells comprising a CAR molecule are administered for at least two, three, four, five, six, seven, eight or more weeks.

In one embodiment, the combination of the kinase inhibitor and the cells expressing a CAR molecule, e.g., a CAR molecule described herein, are administered as a first line treatment for the disease, e.g., the cancer, e.g., the cancer described herein. In another embodiment, the combination of the kinase inhibitor and the cells expressing a CAR molecule, e.g., a CAR molecule described herein, are administered as a second, third, fourth line treatment for the disease, e.g., the cancer, e.g., the cancer described herein.

In embodiments, any of the methods described herein further comprise performing lymphodepletion on a subject, e.g., prior to administering the one or more cells that express a CAR molecule described herein, e.g., a CAR molecule that binds CD19 or CD123. The lymphodepletion can comprise, e.g., administering one or more of melphalan, cytoxan, cyclophosphamide, and fludarabine.

### Subject

In embodiments, the subject is (e.g., is identified as) at risk of developing, has, or is diagnosed with CRS.

In embodiments, the subject has been, is being, or will be administered a CAR therapy, e.g., a CAR therapy described herein. In embodiments, the subject has been, is being, or will be administered a CAR123-expressing cell or a CAR19-expressing cell.

In embodiments, the method comprises identifying (and optionally selecting) a subject i) at risk of developing CRS; or ii) having CRS.

In embodiments, the method comprises selecting the subject for administration of the inhibitor (e.g., JAK-STAT inhibitor or BTK inhibitor). In embodiments, the subject is selected based on (i) his or her risk of developing CRS, (ii) his or her diagnosis of CRS, and/or (iii) whether he or she has been, is being, or will be administered a CAR therapy (e.g., a CAR therapy described herein, e.g., CAR19 therapy, e.g., CTL019; or a CD123 CAR therapy). In embodiments, the subject is selected for administration of the JAK-STAT or BTK inhibitor if the subject is diagnosed with CRS, e.g., severe or non-severe CRS. In embodiments, the subject is selected for administration of the JAK-STAT or BTK inhibitor if the subject is at risk of (e.g., identified as at risk of) developing CRS. In embodiments, the subject is selected for administration of the JAK-STAT or BTK inhibitor if the subject has been, is being, or will be administered a CAR therapy (e.g., a CAR therapy described herein, e.g., CAR19 therapy, e.g., CTL019; or a CAR123 therapy).

### Subject at Risk for CRS

In embodiments, the subject is identified as at risk for CRS if the subject has a high tumor burden, e.g., prior to administration of a CAR therapy (e.g., a CAR therapy described herein).

In embodiments, the subject is identified as at risk for CRS by acquiring a CRS risk status for the subject, wherein said CRS risk status comprises a measure of one, two, three, four, five, six, seven, eight, nine, ten, or more (all) of the following:

(i) the level or activity of sgp130 or IFN-gamma or a combination thereof, in the subject, e.g., in a sample (e.g., a blood sample), e.g., wherein the subject is an adult or pediatric subject;

(ii) the level or activity of sgp130, IFN-gamma, or IL1Ra, or a combination thereof (e.g., a combination of any two or all three of sgp130, IFN-gamma, and IL1Ra), in the subject, e.g., a sample (e.g., a blood sample), e.g., wherein the subject is an adult or pediatric subject;

(iii) the level or activity of sgp130 or IFN-gamma or a combination thereof, in the subject, e.g., in a sample (e.g., a blood sample), and the level of bone marrow disease in the subject, e.g., wherein the subject is a pediatric subject;

(iv) the level or activity of sgp130, IFN-gamma, or MIP1-alpha, or a combination thereof (e.g., a combination of any two or all three of sgp130, IFN-gamma, and MIP1-alpha), in the subject, e.g., in a sample (e.g., a blood sample), e.g., wherein the subject is a pediatric subject,

(v) the level or activity of sgp130, MCP1, or eotaxin, or a combination thereof (e.g., a combination of any two or all three of sgp130, MCP1, or eotaxin), in the subject, e.g., in a sample (e.g., a blood sample), e.g., wherein the subject is an adult or a pediatric subject;

(vi) the level or activity of IL-2, eotaxin, or sgp130, or a combination thereof (e.g., a combination of any two or all three of IL-2, eotaxin, or sgp130), in the subject, e.g., in a sample (e.g., a blood sample), e.g., wherein the subject is an adult or a pediatric subject;

(vii) the level or activity of IFN-gamma, IL-2, or eotaxin, or a combination thereof (e.g., a combination of any two or all three of IFN-gamma, IL-2, or eotaxin), in the subject, e.g., in a sample (e.g., a blood sample), e.g., wherein the subject is a pediatric subject;

(viii) the level or activity of IL-10 and the level of disease burden in the subject, or a combination thereof in a subject, e.g., in a sample (e.g., a blood sample), e.g., wherein the subject is a pediatric subject;

(ix) the level or activity of IFN-gamma or IL-13, or a combination thereof, in the subject, e.g., wherein the subject is a pediatric subject; or

(x) the level or activity of IFN-gamma, IL-13, or MIP1-alpha, or a combination thereof (e.g., a combination of any two or all three of IFN-gamma, IL-13, and MIP1-alpha), in a sample (e.g., a blood sample), e.g., wherein the subject is a pediatric subject; or

(xi) the level or activity of IFN-gamma or MIP1-alpha, or a combination thereof, in a sample (e.g., a blood sample), e.g., wherein the subject is a pediatric subject;

wherein the CRS risk status is indicative of the subject's risk for developing CRS, e.g., severe CRS.

Any of the aforesaid methods can further comprise, responsive to a determination of the CRS risk status, performing one, two, or more (all) of:

identifying the subject as being at high risk of developing severe CRS or at low risk of developing severe CRS;

administering a BTK inihibitor (e.g., ibrutinib) or a JAK-STAT inhibitor (e.g., ruxolitinib);

administering an altered dosing of the CAR-expressing cell therapy;

altering the schedule or time course of the CAR-expressing cell therapy;

administering a therapy to treat CRS, e.g., a therapy chosen from one or more of: an IL-6 inhibitor (e.g., an anti-IL6 receptor inhibitor, e.g., tocilizumab), a vasoactive medication, an immunosuppressive agent, a corticosteroid, or mechanical ventilation; and/or

administering an alternative therapy, e.g., for a subject at high risk of developing severe CRS, e.g., a standard of care for a particular cancer type.

In some embodiments of the methods, the CRS risk status comprises a measure of the level or activity of sgp130, IFN-gamma, or IL-13, or a combination thereof (e.g., a combination of any two or all three of sgp130, IFN-gamma, and IL-13), in the subject, e.g., in a sample (e.g., a blood sample), e.g., wherein the subject is an adult or pediatric subject.

In some embodiments of the methods, the CRS risk status is indicative of whether the subject is at high risk or low risk of developing severe CRS. For example, the CRS can be of clinical grade 1-3, or can be severe CRS of clinical grade 4-5.

In some embodiments, the methods are performed on a subject that does not have a symptom (e.g., a clinical symptom) of CRS, e.g., one or more of low blood pressure or a fever; or severe CRS, e.g., one or more of grade 4 organ toxicity or need for mechanical ventilation.

In some embodiments of the methods, a high level or activity of IFN-gamma, sgp130, MCP1, IL-10, or disease burden, or any combination thereof, is indicative of a high risk of severe CRS. In some embodiments, a low level or activity of IL13, IL1Ra, MIP1α, or eoxtaxin, or any combination thereof, is indicative of a high risk of severe CRS.

In some embodiments of the methods, a subject at high risk of severe CRS has, or is identified as having, a greater level or activity of sgp130 or IFN-gamma or a combination thereof (e.g., in a sample, e.g., a blood sample), e.g., relative to a reference.

In other embodiments of the methods, a subject at high risk of severe CRS has, or is identified as having a greater level or activity of sgp130, a greater level or activity of IFN-gamma, or a lower level or activity of IL1Ra, or a combination thereof (e.g., in a sample, e.g., a blood sample), e.g., relative to a reference. In one embodiment, the subject at high risk of severe CRS is identified as having a greater level or activity of sgp130 and a greater level or activity of IFN-gamma; a greater level or activity of sgp130 and a lower level or activity of IL1Ra; a greater level or activity of IFN-gamma and a lower level or activity of IL1Ra; or a greater level or activity of sgp130, a greater level or activity of IFN-gamma, and a lower level or activity of IL1Ra, e.g., compared to a reference. In some embodiments, the reference is a subject at low risk of severe CRS or a control level or activity. The subject can be a human, e.g., an adult or pediatric subject.

In some embodiments of the methods, a subject at high risk of severe CRS has, or is identified as having, a greater level or activity of sgp130 or IFN-gamma or a combination thereof, and a greater level of bone marrow disease, in the subject (e.g., in a sample, e.g., a blood sample), e.g., relative to a reference, e.g., compared to a subject at low risk of severe CRS or compared to a control level or activity. In one embodiment, the subject at high risk of severe CRS is identified as having a greater level of sgp130 and IFN-gamma; sgp130 and bone marrow disease; IFN-gamma and bone marrow disease; or sgp130, IFN-gamma and bone marrow disease, e.g., compared to a reference, e.g., a subject at low risk of severe CRS or a control level or activity. The subject can be a human, e.g., a pediatric subject.

In some embodiments of the methods, a subject (e.g., a pediatric subject) at high risk of severe CRS is identified as having a greater level or activity of sgp130, a greater level or activity of IFN-gamma, or a lower level or activity of MIP1-alpha, or a combination thereof (e.g., in a sample, e.g., a blood sample) compared to a reference, e.g., a subject at low risk of severe CRS or compared to a control level or activity. In one embodiment, a subject at high risk of severe CRS is identified as having a greater level or activity of sgp130 and a greater level or activity of IFN-gamma; a greater level or activity of sgp130 and a lower level or activity of MIP1-alpha; a greater level or activity of IFN-gamma and a lower level or activity of MIP1-alpha; a greater level or activity of sgp130, a greater level or activity of IFN-gamma, and a lower level or activity of MIP1-alpha, e.g., compared to a reference, e.g., a subject at low risk of severe CRS or compared to a control level or activity.

In some embodiments of the methods, a subject at high risk of severe CRS is identified as having a greater level or activity of sgp130, a greater level or activity of MCP1, or a lower level or activity of eotaxin, or a combination thereof (e.g., in a sample, e.g., a blood sample) compared to a reference, e.g., a subject at low risk of severe CRS or compared to a control level or activity. In some embodiments, a subject at high risk of severe CRS is identified as having: a greater level or activity of sgp130 and a greater level or activity of MCP1, a greater level or activity of sgp130 and a lower level or activity of eotaxin, a greater level or activity of MCP1 and a lower level or activity of eotaxin, a greater level or activity of sgp130, a greater level or activity of MCP1, and a lower level or activity of eotaxin, compared to a reference, e.g., a subject at low risk of severe CRS or compared to a control level or activity.

In some embodiments of the methods, a subject at high risk of severe CRS is identified as having an altered (e.g., greater) level or activity of IL-2, a lower level or activity of eotaxin, or a greater level or activity of sgp130, or a combination thereof (e.g., in a sample, e.g., a blood sample) compared to a reference, e.g., a subject at low risk of severe CRS or compared to a control level or activity. In some embodiments, a subject at high risk of severe CRS is identified as having: an altered (e.g., greater) level or activity of IL-2 and a lower level or activity of eotaxin, an altered (e.g., greater) level or activity of IL-2 and a greater level or activity of sgp130, a lower level or activity of eotaxin and a greater level or activity of sgp130, an altered (e.g., greater) level or activity of IL-2, a lower level or activity of eotaxin, and a greater level or activity of sgp130, compared to a reference, e.g., a subject at low risk of severe CRS or compared to a control level or activity.

In some embodiments of the methods, a subject at high risk of severe CRS is identified as having a greater level or activity of IFN-gamma, an altered (e.g., greater) level or activity of IL-2, or a lower level or activity of eotaxin, or a combination thereof (e.g., in a sample, e.g., a blood sample) compared to a reference, e.g., a subject at low risk of severe CRS or compared to a control level or activity. In some embodiments, the subject is a pediatric subject. In some embodiments, a subject at high risk of severe CRS is identified as having: a greater level or activity of IFN-gamma and an altered (e.g., greater) level or activity of IL-2, a greater level or activity of IFN-gamma and a lower level or activity of eotaxin, an altered (e.g., greater) level or activity of IL-2 and a lower level or activity of eotaxin, a greater level or activity of IFN-gamma, an altered (e.g., greater) level or activity of IL-2, and a lower level or activity of eotaxin, compared to a reference, e.g., a subject at low risk of severe CRS or compared to a control level or activity.

In some embodiments of the methods, a subject at high risk of severe CRS is identified as having a greater level or activity of IL-10 or a greater level of disease burden, or a combination thereof (e.g., in a sample, e.g., a blood sample) compared to a reference, e.g., a subject at low risk of severe CRS or compared to a control level or activity. In some embodiments, the subject is a pediatric subject.

In some embodiments of the methods, a subject at high risk of severe CRS is identified as having a greater level or activity of IFN-gamma or a lower level of IL-13, or a combination thereof (e.g., in a sample, e.g., a blood sample) compared to a reference, e.g., a subject at low risk of severe CRS or compared to a control level or activity. In some embodiments, the subject is a pediatric subject.

In some embodiments of the methods, a subject at high risk of severe CRS is identified as having a greater level or activity of IFN-gamma, a lower level or activity of IL-13, a lower level or activity of MIP1-alpha, or a combination thereof (e.g., in a sample, e.g., a blood sample) compared to a reference, e.g., a subject at low risk of severe CRS or compared to a control level or activity. In some embodiments, the subject is a pediatric subject. In some embodiments, a subject at high risk of severe CRS is identified as having: a greater level or activity of IFN-gamma or a lower level or activity of IL-13, a greater level or activity of IFN-gamma or a lower level or activity of MIP1-alpha, a lower level or activity of IL-13 or a lower level or activity of MIP1-alpha, a greater level or activity of IFN-gamma, a lower level or activity of IL-13, and a lower level or activity of MIP1-alpha, compared to a reference, e.g., a subject at low risk of severe CRS or compared to a control level or activity.

In some embodiments of the methods, a subject at high risk of severe CRS is identified as having a greater level or activity of IFN-gamma or a lower level or activity of MIP1-alpha, or a combination thereof (e.g., in a sample, e.g., a blood sample) compared to a reference, e.g., a subject at low risk of severe CRS or compared to a control level or activity. In some embodiments, the subject is a pediatric subject.

In some embodiments, e.g., in a 3-biomarker panel, e.g., containing IL2, eotaxin, and sgp130, or in a 3-biomarker panel containing IFN-gamma, IL2, and eotaxin (e.g., in pediatric patients) a greater level or activity of IL2 indicates that a subject is at high risk of severe CRS. In other embodiments, e.g., in a 2-biomarker panel, e.g., for pediatric patients, a greater level or activity of IL2 indicates that a subject is at low risk of severe CRS.

In some embodiments of the methods, a greater level of a marker described herein is a level greater than or equal to 1, 2, 5, 10, 20, 50, 100, 200, 500, 1000, 2000, 5000, 10,000, 20,000, 50,000, 100,000, 200,000, or 500,000 pg/ml. In some embodiments, a greater level of sgp130 is greater than or equal to 150,000, 200,000, 210,000, 215,000, 218,000, 218,179, 220,000, 225,000, 230,000, or 250,000 pg/ml. In some embodiments, a greater level of IFN-gamma is greater than or equal to 6, 7, 8, 9, 10, 10.4272, 10.5, 11, 12, 13, 14, 15, 16, 17, 18, 19, 20, 21, 22, 23, 24, 25, 26, 27, 27.6732, 28, 29, 30, 31, 32, 33, 34, 35, 40, 50, 60, 70, 75, 80, 85, 90, 91, 92, 93, 94, 94.8873, 95, 96, 97, 98, 99, 100, 105, 110, 115, or 120 pg/ml. In some embodiments, a greater level of IL-10 is greater than or equal to 5, 6, 7, 8, 9, 10, 11, 11.7457, 12, 13, 14, 15, 16, 17, 18, 19, or 20 pg/ml. In some embodiments, a greater tumor burden is greater than or equal to 25, 30, 35, 40, 45, 50, 51.9, 55, 60, 65, 70, or 75% In some embodiments, a lower level of sgp130, IFN-gamma, IL-10, or tumor burden is a level less than or equal to any of the values in this paragraph.

In some embodiments of the methods, a lower level of a marker described herein is a level greater than or equal to 1, 2, 5, 10, 20, 50, 100, 200, 500, 1000, 2000, 5000, 10,000, 20,000, 50,000, 100,000, 200,000, or 500,000 pg/ml. In some embodiments, a lower level of IL1Ra is less than or equal to 550, 575, 600, 625, 650, 657.987, 675, 700, 720, or 750 pg/ml. In some embodiments, a lower level of MCP1 is less than or equal to 3500, 4000, 4100, 4200, 4300, 4400, 4500, 4600, 4636.52, 4700, 4800, 4900, 5000, or 5500 pg/ml. In some embodiments, a lower level of eotaxin is less than or equal to 20, 21, 22, 23, 24, 25, 26, 27, 28, 29, 29.0902, 30, 31, 32, 33, 34, 35, 36, 37, 38, 39, or 40 pg/ml. In som embodiments, a lower level of MIP1a is less than or equal to 20, 21, 22, 23, 24, 25, 26, 27, 28, 29, 30, 30.1591, 31, 32, 33, 34, 35, 36, 37, 38, 39, or 40 pg/ml. In some embodiments, a greater level of IL1Ra, MCP1, eotaxin, or MIP1a is a level greater than or equal to any of the values in this paragraph. In some embodiments of the methods, the sensitivity is at least 0.75, 0.79, 0.80, 0.82, 0.85, 0.86, 0.90, 0.91, 0.93, 0.95, 0.96, 0.97, 0.98, 0.99, or 1.0. In some embodiments, the specificity is at least 0.75, 0.77, 0.80, 0.85, 0.86, 0.89, 0.90, 0.92, 0.94, 0.95, 0.96, 0.97, 0.98, 0.99, or 1.0. In some embodiments, the PPV is at least 0.62, 0.65, 0.70, 0.71, 0.75, 0.80, 0.82, 0.83, 0.85, 0.90, 0.91, 0.92, 0.95, 0.96, 0.97, 0.98, 0.99, or 1.0. In some embodiments, the NPV is at least 0.80, 0.85, 0.90, 0.92, 0.94, 0.95, 0.96, 0.97, 0.98, 0.99, or 1.0.

In some embodiments of the methods, a measure of eotaxin comprises a measure of one or more of (e.g., two or all of) eotaxin-1, eotaxin-2, and eotaxin-3. In some embodiments, a measure of eotaxin comprises a measure of eotaxin-1 and eotaxin-2, eotaxin-1 and eotaxin-3, or eotaxin-2 and eotaxin-3.

Any of the methods disclosed herein can further include the step of acquiring a measure of the level or activity of one, two, three, four, five, ten, twenty or more of a cytokine chosen from sTNFR2, IP10, sIL1R2, sTNFR1, M1G, VEGF, sILR1, TNFα, IFNα, GCSF, sRAGE, IL4, IL10, IL1R1, IFN-γ, IL6, IL8, sIL2Rα, sgp130, sIL6R, MCP1, MIP1α, MIP1β, or GM-CSF, or a combination thereof, in the subject, e.g., in a sample (e.g., a blood sample) from the subject. In some embodiments, a subject having, or at high risk of having, severe CRS has, or is identified as having, a greater level or activity of one or more (e.g., two, three, four, five, ten, fifteen, twenty, or all) of a cytokine chosen from sTNFR2, IP10, sIL1R2, sTNFR1, M1G, VEGF, sILR1, TNFα, IFNα, GCSF, sRAGE, IL4, IL10, IL1R1, IFN-γ, IL6, IL8, sIL2Rα, sgp130, sIL6R, MCP1, MIP1α, MIP1β, or GM-CSF or a combination thereof, compared to a reference, e.g., a subject at low risk of severe CRS or compared to a control level or activity.

Any of the methods disclosed herein can further include the step of acquiring a measure of the level or activity of one, two, three, four, five, six, seven, eight, or all of a cytokine chosen from IFN-γ, IL10, IL6, IL8, IP10, MCP1, M1G, sIL2Rα, GM-CSF, or TNFα, or or a combination thereof, in the subject, e.g., in a sample (e.g., a blood sample) from the subject. In some embodiments, a subject having, or at high risk of having, severe CRS has, or is identified as having, a greater level or activity of one or more (e.g., two, three, four, five, six, seven, eight, or all) of a cytokine chosen from IFN-γ, IL10, IL6, IL8, IP10, MCP1, M1G, sIL2Rα, GM-CSF, or TNFα or a combination thereof, compared to a reference, e.g., a subject at low risk of severe CRS or compared to a control level or activity.

Any of the methods disclosed herein can further include the step of acquiring a measure of the level or activity of one, two, three, four, five, six, or all of a cytokine chosen from IFN-γ, IL10, IL6, IL8, IP10, MCP1, M1G, or sIL2Rα, or or a combination thereof, in the subject, e.g., in a sample (e.g., a blood sample) from the subject. In some embodiments, a subject having, or at high risk of having, severe CRS has, or is identified as having, a greater level or activity of one or more (e.g., two, three, four, five, six, or all) of a cytokine chosen from IFN-γ, IL10, IL6, IL8, IP10, MCP1, M1G, or sIL2Rα, or a combination thereof, compared to a reference, e.g., a subject at low risk of severe CRS or compared to a control level or activity.

In some embodiments, any the methods disclosed herein can further include the step of determining the level of C-reactive protein (CRP) in a sample (e.g., a blood sample) from the subject. In one embodiment, a subject at low risk of severe CRS has, or is identified as having, a CRP level of less than 7 mg/dL (e.g., 7, 6.8, 6, 5, 4, 3, 2, 1 mg/dL or less). In one embodiment, a subject at high risk of severe CRS has, or is identified as having, a greater level of CRP in a sample (e.g., a blood sample) compared to a subject at low risk of severe CRS or compared to a control level or activity. In one embodiment, the greater level or activity is at least 2-fold greater (e.g., at least 2, 3, 4, 5, 6, 7, 8, 9, 10, 15, 20, 25, 30, 40, 50, 100, 500, 1000-fold or greater) compared to a subject at low risk of severe CRS or compared to a control level or activity.

In other embodiments, the methods, disclosed herein further include the step of selecting or altering the therapy, e.g., the CAR-expressing cell therapy, for the subject, based on the CRS risk status acquired. In embodiments where the CRS risk status acquired is that the subject is at high risk of severe CRS, the therapy is altered such that it is discontinued, or a subsequent (e.g., second, third, or fourth) dose of the therapy (e.g., the CAR-expressing cells) is at a lower dose than the previous dose. In other embodiments, a subsequent (e.g., second, third, or fourth) dose of CAR-expressing cells comprises a different CAR or different cell type than the previous CAR-expressing cell therapy administered to the subject.

In other embodiments of the methods, the measure of one or more of biomarkers (e.g., one or more biomarkers of (i)-(xi)) is obtained from a sample (e.g., a blood sample) acquired from the subject. In some embodiments, the subject, e.g., a sample from the subject, is evaluated while receiving the CAR-expressing cell therapy. In other embodiments, the subject, e.g., a sample from the subject, is evaluating after receiving the CAR-expressing cell therapy. For example, the subject, e.g., a sample from the subject, is evaluated 10 days or less (e.g., 1-10 days, 1-9 days, 1-8 days, 1-7 days, 1-6 days, 1-5 days, 1-4 days, 1-3 days, or 1-2 days, 5 days or less, 4 days or less, 3 days or less, 2 days or less, 1 day or less, e.g., 1, 3, 5, 10, 12, 15, 20 hours) after infusion with the CAR-expressing cell therapy. In some embodiments, the subject is evaluated 5 days or less, 4 days or less, 3 days or less, 2 days or less, 1 day or less (e.g., but no earlier than 1, 3, 5, 10, 12, 15, 20 hours, after infusion of the CAR-expressing therapy). In other embodiments, the measure of one or more of biomarkers comprises detection of one or more of nucleic acid (e.g., mRNA) levels or protein levels.

In embodiments, the method comprises determining whether a subject has severe CRS. The method includes acquiring a CRS risk status, e.g., in response to an immune cell based therapy, e.g., a CAR-expressing cell therapy (e.g., a CAR19-expressing cell therapy or a CAR123-expressing cell therapy) for the subject, wherein said CRS risk status includes a measure of one, two, or more (all) of the following:

(i) the level or activity of one or more (e.g., 3, 4, 5, 10, 15, 20, or more) cytokines chosen from sTNFR2, IP10, sIL1R2, sTNFR1, M1G, VEGF, sILR1, TNFα, IFNα, GCSF, sRAGE, IL4, IL10, IL1R1, IFN-γ, IL6, IL8, sIL2Rα, sgp130, sIL6R, MCP1, MIP1α, MIP1β, or GM-CSF, or analytes chosen from C-reactive protein (CRP), ferritin, lactate dehydrogenase (LDH), aspartate aminotransferase (AST), or blood urea nitrogen (BUN), alanine aminotransferase (ALT), creatinine (Cr), or fibrinogen, or a combination thereof, in a sample (e.g., a blood sample);

(ii) the level or activity of IL6, IL6R, or sgp130, or a combination thereof (e.g., a combination of any two or all three of IL6, IL6R, and sgp130), in a sample (e.g., a blood sample); or

(iii) the level or activity of IL6, IFN-gamma, or IL2R, or a combination thereof (e.g., a combination of any two or all three of IL6, IFN-gamma, and IL2R), in a sample (e.g., a blood sample);

wherein the value is indicative of the subject's severe CRS status.

In embodiments, an elevated level of the cytokines (i)-(iii), or all analytes except fibrinogen, is indicative of severe CRS. In embodiments, low fibrinogen is indicative of severe CRS.

### Compositions and Compositions for Use

In another aspect, the disclosure features a composition (e.g., one or more dosage formulations, combinations, or one or more pharmaceutical compositions) comprising a cell expressing a CAR described herein (e.g., CD123 CAR) and an inhibitor (e.g., JAK-STAT inhibitor, e.g., ruxolitinib) described herein. The CAR-expressing cell and the inhibitor (e.g., JAK-STAT inhibitor) can be in the same or different formulation or pharmaceutical composition. The CAR-expressing cell and the one or more kinase inhibitors can be present in a single dose form, or as two or more dose forms.

In embodiments, the compositions disclosed herein are for use as a medicament.

In embodiments, the compositions disclosed herein are used in the treatment of a disease associated with expression of an antigen described herein, e.g., a B-cell antigen (e.g., CD123 or CD19).

In another aspect, the disclosure features a composition (e.g., one or more dosage formulations, combinations, or one or more pharmaceutical compositions) comprising a cell expressing a CAR described herein (e.g., CD123 CAR) and an inhibitor (e.g., JAK-STAT inhibitor) described herein, for use in a method of treating (or in the preparation of a medicament for treating) a disease associated with expression of an antigen (e.g., B cell antigen, e.g., CD123 or CD19), e.g., a cancer described herein.

In another aspect, the disclosure features a composition (e.g., one or more dosage formulations, combinations, or one or more pharmaceutical compositions) comprising a cell expressing a CAR described herein (e.g., CD123 CAR or CD19 CAR) and an inhibitor (e.g., JAK-STAT inhibitor or BTK inhibitor) described herein, for use in a method of preventing CRS in a subject.

In another aspect, the invention pertains to a cell expressing a CAR molecule described herein for use as a medicament in combination with a kinase inhibitor, e.g., a kinase inhibitor described herein (e.g., a BTK inhibitor such as ibrutinib, or JAK-STAT inhibitor such as ruxolitinib), e.g., to prevent CRS in a subject. In another aspect, the invention pertains to a kinase inhibitor described herein (e.g., a BTK inhibitor such as ibrutinib, or JAK-STAT inhibitor such as ruxolitinib) for use as a medicament in combination with a cell expressing a CAR molecule described herein, e.g., to prevent CRS in a subject.

In another aspect, the invention pertains to a cell expressing a CAR molecule described herein for use in combination with a kinase inhibitor, e.g., a kinase inhibitor described herein (e.g., a BTK inhibitor such as ibrutinib, or JAK-STAT inhibitor such as ruxolitinib), in the treatment of a disease expressing the B-cell antigen (e.g., CD19 or CD123).

In another aspect, the invention pertains to a kinase inhibitor described herein (e.g., a BTK inhibitor such as ibrutinib, or JAK-STAT inhibitor such as ruxolitinib), for use in combination with a cell expressing a CAR molecule described herein, in the treatment of a disease expressing the B-cell antigen (e.g., CD19 or CD123).

In another aspect, the invention pertains to a kinase inhibitor described herein (e.g., a BTK inhibitor such as ibrutinib, or JAK-STAT inhibitor such as ruxolitinib), for use in combination with a cell expressing a CAR molecule described herein, in the reduction of one or more side effects of a CAR therapy described herein.

In another aspect, the invention pertains to a cell expressing a CAR molecule described herein for use (e.g., as a medicament) in combination with a cytokine, e.g., IL-7, IL-15 and/or IL-21 as described herein. In another aspect, the invention pertains to a cytokine described herein for use (e.g., as a medicament) in combination with a cell expressing a CAR molecule described herein.

In another aspect, the invention pertains to a cell expressing a CAR molecule described herein for use (e.g., as a medicament) in combination with a cytokine, e.g., IL-7, IL-15 and/or IL-21 as described herein, in the treatment of a disease expressing a B cell antigen, e.g., CD123 or CD19. In another aspect, the invention pertains to a cytokine described herein for use (e.g., as a medicament) in combination with a cell expressing a CAR molecule described herein, in the treatment of a disease expressing B cell antigen, e.g., CD123 or CD19.

In some aspects, the present disclosure provides a method of distinguishing between CRS and sepsis in a subject, comprising acquiring a measure of one or more of the following:

(i) the level or activity of one or more of (e.g., 2, 3, 4, 5, 6, 7, 8, 9, 10, 11, 12, 13, 14, 15, or all of) GM-CSF, HGF, IFN-γ, IFN-α, IL-10, IL-15, IL-5, IL-6, IL-8, IP-10, MCP1, MIG, MIP-1β, sIL-2Rα, sTNFRI, and sTNFRII, wherein a level or activity that is higher than a reference is indicative of CRS; or

(ii) the level or activity of one or more of (e.g., 2, 3, 4, 5, 6, or all of) CD163, IL-1β, sCD30, sIL-4R, sRAGE, sVEGFR-1, and sVEGFR-2, wherein a level or activity that is higher than a reference is indicative of sepsis.

In embodiments, the method comprises administering a therapy (e.g., a therapy described herein) to treat CRS if the measure is indicative of CRS. In embodiments, the method comprises administering a therapy to treat sepsis if the measure is indicative of sepsis.

The present disclosures also provides, in some aspects, a kit for distinguishing between CRS and sepsis in a patient, the kit comprising a set of reagents that specifically detects the level or activity of one or more (e.g., 2, 3, 4, 5, 6, 7, 8, 9, 10, 11, 12, 13, 14, 15, 16, 17, 18, 19, 20, 2, 22, or all of) genes or proteins chosen from:

GM-CSF, HGF, IFN-γ, IFN-α, IL-10, IL-15, IL-5, IL-6, IL-8, IP-10, MCP1, MIG, MIP-1β, sIL-2Rα, sTNFRI, sTNFRII, CD163, IL-1β, sCD30, sIL-4R, sRAGE, sVEGFR-1, and sVEGFR-2; and

instructions for using said kit;

wherein said instructions for use provide that if one or more of (e.g., 2, 3, 4, 5, 6, 7, 8, 9, 10, 11, 12, 13, 14, 15, or all of) the detected level or activity of GM-CSF, HGF, IFN-γ, IFN-α, IL-10, IL-15, IL-5, IL-6, IL-8, IP-10, MCP1, MIG, MIP-1β, sIL-2Rα, sTNFRI, or sTNFRII is greater than a reference value, the subject is likely to have CRS,

and/or if one or more of (e.g., 2, 3, 4, 5, 6, or all of) the detected level or activity of CD163, IL-1β, sCD30, sIL-4R, sRAGE, sVEGFR-1, or sVEGFR-2, is greater than a reference value, the subject is likely to have sepsis.

The present disclosure also provides, in some aspects, a reaction mixture comprising:

a set of reagents that specifically detects the level or activity of one or more (e.g., 2, 3, 4, 5, 6, 7, 8, 9, 10, 11, 12, 13, 14, 15, 16, 17, 18, 19, 20, 2, 22, 23, or all of) genes or proteins chosen from: GM-CSF, HGF, IFN-γ, IFN-α, IL-10, IL-15, IL-5, IL-6, IL-8, IP-10, MCP1, MIG, MIP-1β, sIL-2Rα, sTNFRI, sTNFRII, CD163, IL-1β, sCD30, sIL-4R, sRAGE, sVEGFR-1, and sVEGFR-2, and

a biological sample, e.g., a blood sample.

In embodiments, the biological sample is from a subject treated with a CAR-expressing cell therapy and/or having a symptom of CRS and/or sepsis.

The present disclosure also provides, in certain aspects, a method of identifying sepsis in a subject, comprising acquiring a measure of one or more of the following:

(i) the level or activity of one or more of (e.g., 2, 3, 4, 5, 6, 7, 8, 9, 10, 11, 12, 13, 14, 15, 16, 17, 18, 19, 20, 21, 22, or all of) ANG2, GCSF, IFNα, IL1RA, IL4, IL6, MIG, MIP1α, PTX3, TNFα, sCD163, sCD30, sIL-1RI, sIL-1RII, sIL-2Rα, sIL-4R, sRAGE, sTNFRI, sTNFRII, sVEGFR1, sVEGFR2, sVEGFR3, and VEGF, wherein a level or activity that is greater relative to a reference is indicative of sepsis;

(ii) the level or activity of one or more of (e.g., both of) IL13 and RANTES, wherein a level or activity that is lower relative to a reference is indicative of sepsis.

In some aspects, the present disclosure provides a method of treating one or more of a neurological toxicity, CRS, or posterior reversible encephalopathy syndrome (PRES), comprising administering to a subject in need thereof a therapeutically effective amount of cyclophosphamide. In related aspects, the present disclosure provides cyclophosphamide for use in treating neurological toxicity, CRS, or posterior reversible encephalopathy syndrome (PRES). In embodiments, the administration of cyclophosphamide is subsequent to a cell-based therapy, e.g., a cell-based therapy for cancer, a CD19-inhibiting therapy, or a CD19-depleting therapy, or the subject has been previously treated with a cell-based therapy, e.g., a cell-based therapy for cancer, a CD19-inhibiting therapy, or a CD19-depleting therapy. In embodiments, the administration of cyclophosphamide is prior to, at the same time as, or after the cell-based therapy.

In embodiments, the patient has, or is identified as having, CRS, PRES, or both. In some embodiments, the subject has been treated with a CD19 inhibiting or depleting therapy. In some embodiments, the CD19 inhibitor is a CD19 antibody, e.g., a CD19 bispecific antibody (e.g., a bispecific T cell engager that targets CD19, e.g., blinatumomab). In some embodiments, the therapy comprises a CAR-expressing cell, e.g., an anti-BCMA CAR or anti-CD19 CAR. In embodiments, the subect suffers from a neurological toxicity, e.g., focal deficits (e.g., cranial nerve palsy or hemiparesis) or global abnormalities (e.g., generalized seizures, confusion), or status epilepticus. In embodiments, the subject does not have any clinical symptoms of CRS. In embodiments, the subject has one or more clinical symptoms of CRS. In embodiments, the subject has, or is identified as having, elevated IL-6 relative to a reference, e.g., to the subject's level of IL-6 prior to therapy with a CAR-expressing cell. In embodiments, the subject has, or is identified as having, elevated serum levels of a cytokine associated with CRS (e.g., IL-6 and/or IL-8) relative to a reference. In embodiments, the subject has, or is identified as having, elevated levels of a cytokine associated with CRS (e.g., CSF IL-6 and/or IL-8) relative to a reference. In embodiments, the subject is treated or has been treated with a therapy for CRS such as tocilizumab or a corticosteroid (e.g., (methylprednisolone, hydrocortisone, or both). In embodiments, the subject has, or is identified as having, an increase in circulating, activated CR-expressing cells. In embodiments, the subject has, or is identified as having, CAR-expressing cells in the CSF.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc, are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1A is a schematic illustrating the experiments performed as described in Example 1, e.g., to generate a mouse model of CRS after CART. FIG. 1B is a graph showing the expansion of CART cells after AML injection. FIG. 1C is a survival curve showing the survival of mice after a high dose of CART123. FIG. 1D is a panel of graphs showing the levels of various cytokines in mice treated with high dose CART123.

FIG. 2A is a schematic illustrating the experiments performed as described in Example 1, e.g., to determine the effect of ruxolitinib on CRS after CART therapy. FIG. 2B is a graph showing the change in weight of the mice, as measured by % change from baseline, which is plotted on the y axis against time on the x axis. FIG. 2C is a graph showing the disease burden, as measured by leukemic cells/ul (huCD45 dim cells), from serial retro-orbital bleedings, which is plotted on the y axis against time on the x axis. FIG. 2D is a graph showing the change in weight of the mice when treated with ruxolitinib. Weight as measured by % change from baseline is plotted on the y axis against time on the x axis. FIG. 2E is a graph showing the absolute CD3+ cell counts from serial retro-orbital bleedings from the mice. Serial retro-orbital bleedings were performed at the indicated time points on x-axis. Absolute CD3+ cell count is plotted on the Y axis. FIG. 2F is a set of graphs showing the level of inflammatory cytokines from mouse serum obtained by retro-orbital bleeding of the mice one week after CAR123 injection. FIG. 2G is a survival plot showing the survival of mice treated with 60 mg/kg ruxolitinib in combination with CART123. FIG. 2H is a flow cytometry plot showing an analysis of peripheral blood from surviving mice treated with ruxolitinib at 70 days post AML injection (gated on live human CD45 positive cells).

FIG. 3A is a schematic of the experiments described in Example 2, in particular the generation of a model for CRS after CART19 treatment in B cell neoplasms. FIG. 3B is an image of spleen from a representative mouse sacrificed before T cell treatment, showing high tumor burden. FIG. 3C is a flow cytometry plot showing a high level of circulating neoplastic B cells present in the peripheral blood (PB) at time of randomization (gating strategy: time gate, lymphocytes, single cells, live gate, huCD45+ muCD45−). FIG. 3D is a survival curve showing that mice treated with CART19 experienced a significantly reduced overall survival. FIG. 3E is a panel of graphs showing a Luminex analysis of serum human cytokines, which revealed significantly increased cytokines in PB of mice receiving CART19 as compared as no treatment. For FIGS. 3C-3E, all graphs were representative of two independent experiments (5 mice per group). Student's t-test was used to compare two groups. Survival curves were compared using the log-rank test. Asterisks represent p-values (*=<0.05, **=<0.01, ***=<0.001, ****=<0.0001) and “ns” means “not significant” (p>0.05).

FIG. 4A is a schematic showing the experiments in Example 2, e.g., administration of CART19 in combination with ibrutinib or vehicle in the mouse model generated in Example 2. FIG. 4B is a survival curve showing that mice treated with CART19 plus ibrutinib experienced a significantly increased overall survival. FIG. 4C is a graph showing the number of CD19+ cells in peripheral blood after vehicle or ibrutinib treatment. FIG. 4D is a graph showing that T cell expansion was not negatively affected by ibrutinib treatment (rather, T cell expansion was augmented by ibrutinib treatment). FIG. 4E is a panel of graphs showing the level of serum cytokines from mice treated with CART19 or CART19+ibrutinib analyzed by Luminex; a significant reduction in all the cytokines involved in CRS was observed. FIG. 4F is a panel of graphs showing significant cytokine production in a dose-dependent manner in primary MCL cells incubated for 24 hours with ibrutinib. All graphs in FIGS. 4B-4F are representative of two independent experiments (5 mice per group). Student's t-test was used to compare two groups; in analysis where multiple groups were compared, one-way analysis of variance (ANOVA) was performed with Holm-Sidak correction for multiple comparisons. Survival curves were compared using the log-rank test. Asterisks represent p-values (*=<0.05, **=<0.01, ***=<0.001, ****=<0.0001) and “ns” means “not significant” (p>0.05).

FIG. 5 is a graph showing serum cytokine concentrations in xenograft mice bearing primary pediatric ALL treated with CD19 CAR T cells. NSG mice were given 10⁶ primary ALL and 5×10⁶ autologous CD19 CAR T cells seven days later. Serum was collected 3 days following T cell delivery, and a subgroup of animals was given tocilizumab on days 1 and 3 after T cells. Cytokine concentrations were measured in pg/mL.

FIG. 6 is a graph showing serum cytokine concentrations in xenograft mice bearing an ALL cell line treated with CD19 CAR T cells. NSG mice were engrafted with 10⁶ Nalm-6 ALL cells and seven days later given 5×10⁶ CD19 CAR T cells derived from a normal donor. Serum was collected 3 days following T cell delivery, and a subgroup of animals was given tocilizumab on days 1 and 3 after T cells. Cytokine concentrations are measured in pg/mL.

FIG. 7A-7J are graphs showing cytokine expression after cellular co-culture. T cells, targets and APCs were combined at a ratio of 10:50:1, respectively. Supernatants were collected after 18 hours of co-culture. Cytokine levels are measured in pg/mL. Significant differences are denoted with either a * or **, and represent a p value of <0.05.

FIG. 8A-8E are graphs showing cytokine secretion from co-culture experiments combining monocyte-lineage cells with T cells and targets. Monocyte-lineage cells were differentiated in vitro, and T cells, targets and APCs were combined at a ratio of 10:50:1, respectively. Supernatants were collected at 18 and 48 hours and analyzed for cytokine concentrations, measured in pg/mL.

FIG. 9A-9C are graphs showing transcriptional analysis of isolated cell populations. T cells and targets were separated from APCs using trans-well inserts and co-cultured for 18 hours. 697 RNA transcripts were quantified from each cell population and log counts of each are displayed. Transcriptional profile of (A) CD19 CAR T cells when combined with targets and when combined with targets and pooled monocytes, (B) APCs when combined with targets and when combined with targets and untargeted T cells, and (C) APCs when combined with targets and untargeted T cells, and when combined with targets and targeted T cells.

FIG. 10 is a graph showing transcript profile of activated CD19 CAR T cells and monocyte-lineage APCs. Cells were harvested from trans-well co-culture of CD19 CAR T cells, Nalm-6 leukemia and pooled monocytes after 18 hours. Transcript counts from T cells are displayed in blue, and counts from APCs in red.

FIG. 11A-11C are graphs showing T cell degranulation in the presence of APCs. T cells expressing either (A) no CAR molecule, (B) GD2-targeted CAR or (C) CD19-targeted CAR were combined with CD19+ target ALL cell line Nalm-6. Degranulation was measured by quantification of CD107a surface expression.

FIG. 12 is a diagram showing NanoString analysis of PBMCs collected from patients with ALL treated with CD19 CAR T cells. Peripheral blood was collected on first day of fever after engineered T cell infusion. The first seven patients had T cells detectable in peripheral blood with no detectable ALL, while the last three patients had only ALL cells and no detectable T cells.

FIG. 13 is a set of images showing microscopic analysis of peripheral blood T cells collected at time of first fever after CD19 CAR T cell infusion in patients with acute lymphoblastic leukemia. Images captured at 1000× magnification.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “Chimeric Antigen Receptor” or alternatively a “CAR” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains, e.g., as provided in an RCAR as described herein.

In one aspect, the stimulatory molecule of the CAR is the zeta chain associated with the T cell receptor complex. In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 4-1BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., aa scFv) during cellular processing and localization of the CAR to the cellular membrane.

A CAR that comprises an antigen binding domain (e.g., a scFv, a single domain antibody, or TCR (e.g., a TCR alpha binding domain or TCR beta binding domain)) that specifically binds a specific tumor marker X, wherein X can be a tumor marker as described herein, is also referred to as XCAR. For example, a CAR that comprises an antigen binding domain that specifically binds CD123 is referred to as CD123 CAR or CAR123. For example, a CAR that comprises an antigen binding domain that specifically binds CD19 is referred to as CD19 CAR or CAR19. In some embodiments, the CAR comprises a CTL019 CAR as described herein. The CAR can be expressed in any cell, e.g., an immune effector cell as described herein (e.g., a T cell or an NK cell).

A therapy that comprises a CAR-expressing cell is referred to herein as a CAR-therapy. For example, a therapy that comprises a CD123 CAR-expressing cell, or a CD19 CAR is referred to herein as a CD123 CAR therapy or a CD19 CAR therapy, respectively.

The term “signaling domain” refers to the functional portion of a protein which acts by transmitting information within the cell to regulate cellular activity via defined signaling pathways by generating second messengers or functioning as effectors by responding to such messengers.

As used herein, the terms “alpha subunit of the IL-3 receptor,” “IL3Rα,” “CD123,” “IL3Rα chain” and “IL3Rα subunit” refer interchangeably to an antigenic determinant known to be detectable on leukemia precursor cells. The human and murine amino acid and nucleic acid sequences can be found in a public database, such as GenBank, UniProt and Swiss-Prot. For example, the amino acid sequence of human IL3Rα can be found at Accession No. NP 002174 and the nucleotide sequence encoding of the human IL3Rα can be found at Accession No. NM 005191. In one aspect the antigen-binding portion of the CAR recognizes and binds an epitope within the extracellular domain of the CD123 protein. In one aspect, the CD123 protein is expressed on a cancer cell. As used herein, “CD123” includes proteins comprising mutations, e.g., point mutations, fragments, insertions, deletions and splice variants of full length wild-type CD123.

As used herein, the term “CD19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. As used herein, “CD19” includes proteins comprising mutations, e.g., point mutations, fragments, insertions, deletions and splice variants of full length wild-type CD19. CD19 is expressed on most B lineage cancers, including, e.g., acute lymphoblastic leukaemia, chronic lymphocyte leukaemia and non-Hodgkin lymphoma. Other cells with express CD19 are provided below in the definition of “disease associated with expression of CD19.” It is also an early marker of B cell progenitors. See, e.g., Nicholson et al. Mol. Immun. 34 (16-17): 1157-1165 (1997). In one aspect the antigen-binding portion of the CART recognizes and binds an antigen within the extracellular domain of the CD19 protein. In one aspect, the CD19 protein is expressed on a cancer cell.

As used herein, the term “CD20” refers to an antigenic determinant known to be detectable on B cells. Human CD20 is also called membrane-spanning 4-domains, subfamily A, member 1 (MS4A1). The human and murine amino acid and nucleic acid sequences can be found in a public database, such as GenBank, UniProt and Swiss-Prot. For example, the amino acid sequence of human CD20 can be found at Accession Nos. NP_690605.1 and NP_068769.2, and the nucleotide sequence encoding transcript variants 1 and 3 of the human CD20 can be found at Accession No. NM_152866.2 and NM_021950.3, respectively. In one aspect the antigen-binding portion of the CAR recognizes and binds an antigen within the extracellular domain of the CD20 protein. In one aspect, the CD20 protein is expressed on a cancer cell.

As used herein, the term “CD22,” refers to an antigenic determinant known to be detectable on leukemia precursor cells. The human and murine amino acid and nucleic acid sequences can be found in a public database, such as GenBank, UniProt and Swiss-Prot. For example, the amino acid sequences of isoforms 1-5 human CD22 can be found at Accession Nos. NP 001762.2, NP 001172028.1, NP 001172029.1, NP 001172030.1, and NP 001265346.1, respectively, and the nucleotide sequence encoding variants 1-5 of the human CD22 can be found at Accession No. NM 001771.3, NM 001185099.1, NM 001185100.1, NM 001185101.1, and NM 001278417.1, respectively. In one aspect the antigen-binding portion of the CAR recognizes and binds an antigen within the extracellular domain of the CD22 protein. In one aspect, the CD22 protein is expressed on a cancer cell.

As used herein, the term “ROR1” refers to an antigenic determinant known to be detectable on leukemia precursor cells. The human and murine amino acid and nucleic acid sequences can be found in a public database, such as GenBank, UniProt and Swiss-Prot. For example, the amino acid sequences of isoforms land 2 precursors of human ROR1 can be found at Accession Nos. NP_005003.2 and NP_001077061.1, respectively, and the mRNA sequences encoding them can be found at Accession Nos. NM_005012.3 and NM_001083592.1, respectively. In one aspect the antigen-binding portion of the CAR recognizes and binds an antigen within the extracellular domain of the ROR1 protein. In one aspect, the ROR1 protein is expressed on a cancer cell.

As used herein, the term “CD33” refers to the Cluster of Differentiation 33 protein, which is an antigenic determinant detectable on leukemia cells as well on normal precursor cells of the myeloid lineage. The human and murine amino acid and nucleic acid sequences can be found in a public database, such as GenBank, UniProt and Swiss-Prot. For example, the amino acid sequence of human CD33 can be found as UniProt/Swiss-Prot Accession No. P20138 and the nucleotide sequence encoding of the human CD33 can be found at Accession No. NM_001772.3. In one aspect the antigen-binding portion of the CAR recognizes and binds an epitope within the extracellular domain of the CD33 protein or fragments thereof. In one aspect, the CD33 protein is expressed on a cancer cell. As used herein, “CD33” includes proteins comprising mutations, e.g., point mutations, fragments, insertions, deletions and splice variants of full length wild-type CD33.

As used herein, the term “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells, and plasma cells. Its ligand is called B-cell activator of the TNF family (BAFF) and a proliferation inducing ligand (APRIL). BCMA is involved in mediating the survival of plasma cells for mataining long-term humoral immunity. The gene for BCMA is encoded on chromosome 16 producing a primary mRNA transcript of 994 nucleotides in length (NCBI accession NM_001192.2) that encodes a protein of 184 amino acids (NP_001183.2). A second antisense transcript derived from the BCMA locus has been described, which may play a role in regulating BCMA expression. (Laabi Y. et al., Nucleic Acids Res., 1994, 22:1147-1154). Additional transcript variants have been described with unknown significance (Smirnova A S et al. Mol Immunol., 2008, 45(4):1179-1183. A second isoform, also known as TV4, has been identified (Uniprot identifier Q02223-2). As used herein, “BCMA” includes proteins comprising mutations, e.g., point mutations, fragments, insertions, deletions and splice variants of full length wild-type BCMA.

As used herein, the term “CLL-1” refers to C-type lectin-like molecule-1, which is an antigenic determinant detectable on leukemia precursor cells and on normal immune cells. C-type lectin-like-1 (CLL-1) is also known as MICL, CLEC12A, CLEC-1, Dendritic Cell-Associated Lectin 1, and DCAL-2. The human and murine amino acid and nucleic acid sequences can be found in a public database, such as GenBank, UniProt and Swiss-Prot. For example, the amino acid sequence of human CLL-1 can be found as UniProt/Swiss-Prot Accession No. Q5QGZ9 and the nucleotide sequence encoding of the human CLL-1 can be found at Accession Nos. NM 001207010.1, NM 138337.5, NM 201623.3, and NM 201625.1. In one embodiment, the antigen-binding portion of the CAR recognizes and binds an epitope within the extracellular domain of the CLL-1 protein or a fragment thereof. In one embodiment, the CLL-1 protein is expressed on a cancer cell.

The term “EGFR” refers to any mammalian mature full-length epidermal growth factor receptor, including human and non-human forms. The 1186 amino acid human EGFR is described in Ullrich et al., Nature 309:418-425 (1984)) and GenBank Accession No. AF125253 and SwissProt Acc No P00533-2.

The term “EGFRvIII” refers to Epidermal growth factor receptor variant III. EGFRvIII is the most common variant of EGFR observed in human tumors but is rarely observed in normal tissue. This protein results from the in-frame deletion of exons 2-7 and the generation of a novel glycine residue at the junction of exons 1 and 8 within the extra-cellular domain of the EGFR, thereby creating a tumor specific epitope. EGFRvIII is expressed in 24% to 67% of GBM, but not in normal tissues. EGFRvIII is also known as type III mutant, delta-EGFR, EGFRde2-7, and EGFR and is described in U.S. Pat. Nos. 6,455,498, 6,127,126, 5,981,725, 5,814,317, 5,710,010, 5,401,828, and 5,212,290. Expression of EGFRvIII may result from a chromosomal deletion, and may also result from aberrant alternative splicing. See Sugawa et al., 1990, Proc. Natl. Acad. Sci. 87:8602-8606.

As used herein, the term “mesothelin” refers to the 40-kDa protein, mesothelin, which is anchored at the cell membrane by a glycosylphosphatidyl inositol (GPI) linkage and an amino-terminal 31-kDa shed fragment, called megkaryocyte potentiating factor (MPF). Both fragments contain N-glycosylation sites. The term also refers to a soluble splice variant of the 40-kDa carboxyl-terminal fragment also called “soluble mesothelin/MPF-related”. Preferably, the term refers to a human mesothelin of GenBank accession number AAH03512.1, and naturally cleaved portions thereof, e.g., as expressed on a cell membrane, e.g., a cancer cell membrane.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)₂, and Fv fragments, scFv antibody fragments, linear antibodies, single domain antibodies such as sdAb (either VL or VH), camelid VHH domains, and multi-specific antibodies formed from antibody fragments such as a bivalent fragment comprising two Fab fragments linked by a disulfide brudge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The portion of the CAR composition of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized or human antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises a scFv.

As used herein, the term “binding domain” or “antibody molecule” (also referred to herein as “anti-target (e.g., CD123) binding domain”) refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “anti-tumor effect” refers to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the peptides, polynucleotides, cells and antibodies in prevention of the occurrence of cancer in the first place.

The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species. The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound of the present invention and a combination partner, are both administered to a patient simultaneously in the form of a single entity or dosage. The term “non-fixed combination” means that the active ingredients, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of a B-cell antigen” includes, but is not limited to, a disease associated with expression of one or more of CD19, CD20, CD22 or ROR1, or a condition associated with cells which express, or at any time expressed, one or more of CD19, CD20, CD22 or ROR1, including, e.g., proliferative diseases such as a cancer or malignancy or a precancerous condition such as a myelodysplasia, a myelodysplastic syndrome or a preleukemia; or a noncancer related indication associated with cells which express one or more of CD19, CD20, CD22 or ROR1. For the avoidance of doubt, a disease associated with expression of the B-cell antigen may include a condition associated with cells which do not presently express the B-cell antigen, e.g., because the antigen expression has been downregulated, e.g., due to treatment with a molecule targeting the B-cell antigen, e.g., a B-cell targeting CAR, but which at one time expressed the antigen. The phrase “disease associated with expression of a B-cell antigen” includes a disease associated with expression of CD19, as described herein.

The phrase “disease associated with expression of CD19” includes, but is not limited to, a disease associated with expression of CD19 or condition associated with cells which express, or at any time expressed, CD19 including, e.g., proliferative diseases such as a cancer or malignancy or a precancerous condition such as a myelodysplasia, a myelodysplastic syndrome or a preleukemia; or a noncancer related indication associated with cells which express CD19. For the avoidance of doubt, a disease associated with expression of CD19 may include a condition associated with cells which do not presently express CD19, e.g., because CD19 expression has been downregulated, e.g., due to treatment with a molecule targeting CD19, e.g., a CD19 CAR, but which at one time expressed CD19. In one aspect, a cancer associated with expression of CD19 is a hematological cancer. In one aspect, the hematolical cancer is a leukemia or a lymphoma. In one aspect, a cancer associated with expression of CD19 includes cancers and malignancies including, but not limited to, e.g., one or more acute leukemias including but not limited to, e.g., B-cell acute Lymphoid Leukemia (BALL), T-cell acute Lymphoid Leukemia (TALL), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of CD19 comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma (MCL), Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further diseases associated with expression of CD19 expression include, but not limited to, e.g., atypical and/or non-classical cancers, malignancies, precancerous conditions or proliferative diseases associated with expression of CD19. Non-cancer related indications associated with expression of CD19 include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels or reduced levels. In an embodiment, the tumor antigen-expressing cells produced detectable levels of a tumor antigen protein at one point, and subsequently produced substantially no detectable tumor antigen protein.

The phrase “disease associated with expression of CD123” as used herein includes but is not limited to, a disease associated with expression of CD123 or condition associated with a cell which expresses CD123 (e.g., wild-type or mutant CD123) including, e.g., a proliferative disease such as a cancer or malignancy; a precancerous condition such as a myelodysplasia, a myelodysplastic syndrome or a preleukemia; or a non-cancer related indication associated with a cell which expresses CD123 (e.g., wild-type or mutant CD123). In one aspect, a cancer associated with expression of CD123 (e.g., wild-type or mutant CD123) is a hematological cancer. In one aspect, the disease includes AML, ALL, hairy cell leukemia, Prolymphocytic leukemia, Chronic myeloid leukemia (CML), Hodgkin lymphoma, Blastic plasmacytoid dendritic cell neoplasm, lymphoblastic B-cell leukemia (B-cell acute lymphoid leukemia, BALL), acute lymphoblastic T-cell leukemia (T-cell acute lymphoid leukemia (TALL); myelodysplastic syndrome; a myeloproliferative neoplasm; a histiocytic disorder (e.g., a mast cell disorder or a blastic plasmacytoid dendritic cell neoplasm); a mast cell disorder, e.g., systemic mastocytosis or mast cell leukemia, and the like. Further disease associated with expression of CD123 expression include, but are not limited to, e.g., atypical and/or non-classical cancers, malignancies, precancerous conditions or proliferative diseases associated with expression of CD123. Non-cancer related indications associated with expression of CD123 may also be included.

The phrase “disease associated with expression of CD33” as used herein includes but is not limited to, a disease associated with a cell which expresses CD33 (e.g., wild-type or mutant CD33) or condition associated with a cell which expresses CD33 (e.g., wild-type or mutant CD33) including, e.g., a proliferative disease such as a cancer or malignancy or a precancerous condition such as a myelodysplasia, a myelodysplastic syndrome or a preleukemia; or a noncancer related indication associated with a cell which expresses CD33 (e.g., wild-type or mutant CD33). For the avoidance of doubt, a disease associated with expression of CD33 may include a condition associated with a cell which do not presently express CD33, e.g., because CD33 expression has been downregulated, e.g., due to treatment with a molecule targeting CD33, e.g., a CD33 inhibitor described herein, but which at one time expressed CD33. In one aspect, a cancer associated with expression of CD33 (e.g., wild-type or mutant CD33) is a hematological cancer. In one aspect, a hematological cancer includes but is not limited to acute myeloid leukemia (AML), myelodysplasia and myelodysplastic syndrome, myelofibrosis and myeloproliferative neoplasms, acute lymphoid leukemia (ALL), hairy cell leukemia, Prolymphocytic leukemia, chronic myeloid leukemia (CML), Blastic plasmacytoid dendritic cell neoplasm, and the like. Further disease associated with expression of CD33 (e.g., wild-type or mutant CD33) expression include, but are not limited to, e.g., atypical and/or non-classical cancers, malignancies, precancerous conditions or proliferative diseases associated with expression of CD33 (e.g., wild-type or mutant CD33). Non-cancer related indications associated with expression of CD33 (e.g., wild-type or mutant CD33) may also be included. In embodiments, a non-cancer related indication associated with expression of CD33 includes but is not limited to, e.g., autoimmune disease, (e.g., lupus), inflammatory disorders (allergy and asthma) and transplantation. In some embodiments, the tumor antigen-expressing cell expresses, or at any time expressed, mRNA encoding the tumor antigen. In an embodiment, the tumor antigen-expressing cell produces the tumor antigen protein (e.g., wild-type or mutant), and the tumor antigen protein may be present at normal levels or reduced levels. In an embodiment, the tumor antigen-expressing cell produced detectable levels of a tumor antigen protein at one point, and subsequently produced substantially no detectable tumor antigen protein.

The phrase “disease associated with expression of BCMA” includes, but is not limited to, a disease associated with a cell which expresses BCMA (e.g., wild-type or mutant BCMA) or condition associated with a cell which expresses BCMA (e.g., wild-type or mutant BCMA) including, e.g., proliferative diseases such as a cancer or malignancy or a precancerous condition such as a myelodysplasia, a myelodysplastic syndrome or a preleukemia; or a noncancer related indication associated with a cell which expresses BCMA (e.g., wild-type or mutant BCMA). For the avoidance of doubt, a disease associated with expression of BCMA may include a condition associated with a cell which does not presently express BCMA, e.g., because BCMA expression has been downregulated, e.g., due to treatment with a molecule targeting BCMA, e.g., a BCMA inhibitor described herein, but which at one time expressed BCMA. In one aspect, a cancer associated with expression of BCMA (e.g., wild-type or mutant BCMA) is a hematological cancer. In one aspect, the hematogical cancer is a leukemia or a lymphoma. In one aspect, a cancer associated with expression of BCMA (e.g., wild-type or mutant BCMA) is a malignancy of differentiated plasma B cells. In one aspect, a cancer associated with expression of BCMA (e.g., wild-type or mutant BCMA) includes cancers and malignancies including, but not limited to, e.g., one or more acute leukemias including but not limited to, e.g., B-cell acute Lymphoid Leukemia (“BALL”), T-cell acute Lymphoid Leukemia (“TALL”), acute lymphoid leukemia (ALL); one or more chronic leukemias including but not limited to, e.g., chronic myelogenous leukemia (CML), Chronic Lymphoid Leukemia (CLL).

Additional cancers or hematologic conditions associated with expression of BMCA (e.g., wild-type or mutant BCMA) comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. In some embodiments, the cancer is multiple myeloma, Hodgkin's lymphoma, non-Hodgkin's lymphoma, or glioblastoma. In embodiments, a disease associated with expression of BCMA includes a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). Further diseases associated with expression of BCMA (e.g., wild-type or mutant BCMA) expression include, but not limited to, e.g., atypical and/or non-classical cancers, malignancies, precancerous conditions or proliferative diseases associated with expression of BCMA (e.g., wild-type or mutant BCMA), e.g., a cancer described herein, e.g., a prostate cancer (e.g., castrate-resistant or therapy-resistant prostate cancer, or metastatic prostate cancer), pancreatic cancer, or lung cancer.

Non-cancer related conditions that are associated with BCMA (e.g., wild-type or mutant BCMA) include viral infections; e.g., HIV, fungal invections, e.g., C. neoformans; autoimmune disease; e.g. rheumatoid arthritis, system lupus erythematosus (SLE or lupus), pemphigus vulgaris, and Sjogren's syndrome; inflammatory bowel disease, ulcerative colitis; transplant-related allospecific immunity disorders related to mucosal immunity; and unwanted immune responses towards biologics (e.g., Factor VIII) where humoral immunity is important. In embodiments, a non-cancer related indication associated with expression of BCMA includes but is not limited to, e.g., autoimmune disease, (e.g., lupus), inflammatory disorders (allergy and asthma) and transplantation. In some embodiments, the tumor antigen-expressing cell expresses, or at any time expressed, mRNA encoding the tumor antigen. In an embodiment, the tumor antigen-expressing cell produces the tumor antigen protein (e.g., wild-type or mutant), and the tumor antigen protein may be present at normal levels or reduced levels. In an embodiment, the tumor antigen-expressing cell produced detectable levels of a tumor antigen protein at one point, and subsequently produced substantially no detectable tumor antigen protein.

The phrase “disease associated with expression of CLL-1” includes, but is not limited to, a disease associated with a cell which expresses CLL-1 or condition associated with a cell which expresses CLL-1 including, e.g., proliferative diseases such as a cancer or malignancy or a precancerous condition such as a myelodysplasia, a myelodysplastic syndrome or a preleukemia; or a noncancer related indication associated with a cell which expresses CLL-1 (e.g., wild-type or mutant CLL-1). For the avoidance of doubt, a disease associated with expression of CLL-1 may include a condition associated with a cell which do not presently express CLL-1, e.g., because CLL-1 expression has been downregulated, e.g., due to treatment with a molecule targeting CLL-1, e.g., a CLL-1 inhibitor described herein, but which at one time expressed CLL-1. In one aspect, a cancer associated with expression of CLL-1 is a hematological cancer. In one aspect, a hematological cancer includes but is not limited to leukemia (such as acute myelogenous leukemia, chronic myelogenous leukemia, acute lymphoid leukemia, chronic lymphoid leukemia and myelodysplastic syndrome) and malignant lymphoproliferative conditions, including lymphoma (such as multiple myeloma, non-Hodgkin's lymphoma, Burkitt's lymphoma, and small cell- and large cell-follicular lymphoma). Further diseases associated with expression of CLL-1 expression include, but not limited to, e.g., atypical and/or non-classical cancers, malignancies, precancerous conditions or proliferative diseases associated with expression of CLL-1. Non-cancer related indications associated with expression of CLL-1 may also be included. In some embodiments, the tumor antigen-expressing cell expresses, or at any time expressed, mRNA encoding the tumor antigen. In an embodiment, the tumor antigen-expressing cell produces the tumor antigen protein (e.g., wild-type or mutant), and the tumor antigen protein may be present at normal levels or reduced levels. In an embodiment, the tumor antigen-expressing cell produced detectable levels of a tumor antigen protein at one point, and subsequently produced substantially no detectable tumor antigen protein.

The term “disease associated with expression of EGFRvIII” as used herein includes, but is not limited to, a disease associated with expression of EGFRvIII or condition associated with cells which express EGFRvIII including, tumor cells of various cancers such as, e.g., glioblastoma (including glioblastoma stem cells); breast, ovarian, and non-small cell lung carcinomas; head and neck squamous cell carcinoma; medulloblastoma, colorectal cancer, prostate cancer, and bladder carcinoma. Without being bound to a particular theory or mechanism, it is believed that by eliciting an antigen-specific response against EGFRvIII, the CARs disclosed herein provide for one or more of the following: targeting and destroying EGFRvIII-expressing tumor cells, reducing or eliminating tumors, facilitating infiltration of immune cells to the tumor site, and enhancing/extending anti-tumor responses. Because EGFRvIII is not expressed at detectable levels in normal (i.e., non-cancerous) tissue, it is contemplated that the inventive CARs advantageously substantially avoid targeting/destroying normal tissues and cells.

The phrase “disease associated with expression of mesothelin” as used herein includes, but is not limited to, a disease associated with expression of mesothelin or condition associated with cells which express mesothelin including, e.g., proliferative diseases such as a cancer or malignancy or a precancerous condition such as a mesothelial hyperplasia; or a noncancer related indication associated with cells which express mesothelin. Examples of various cancers that express mesothelin include but are not limited to, mesothelioma, ovarian cancer, pancreatic cancer, and the like.

In some embodiments, the tumor antigen (e.g., CD123- or CD19-)-expressing cell expresses, or at any time expressed, mRNA encoding the tumor antigen. In an embodiment, the tumor antigen (e.g., CD123- or CD19-)-expressing cell produces the tumor antigen protein (e.g., wild-type or mutant), and the tumor antigen protein may be present at normal levels or reduced levels. In an embodiment, the tumor antigen (e.g., CD123- or CD19-)-expressing cell produced detectable levels of a tumor antigen protein at one point, and subsequently produced substantially no detectable tumor antigen protein.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation,” refers to a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In one aspect, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12. In a specific CAR of the invention, the intracellular signaling domain in any one or more CARS of the invention comprises an intracellular signaling sequence, e.g., a primary signaling sequence of CD3-zeta. In a specific CAR of the invention, the primary signaling sequence of CD3-zeta is the sequence provided as SEQ ID NO:9, or the equivalent residues from a non-human species, e.g., mouse, rodent, monkey, ape and the like. In a specific CAR of the invention, the primary signaling sequence of CD3-zeta is the sequence as provided in SEQ ID NO:10, or the equivalent residues from a non-human species, e.g., mouse, rodent, monkey, ape and the like.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. The intracellular signaling domain can generate a signal that promotes an immune effector function of the CAR containing cell, e.g., a CART cell or CAR-expressing NK cell. Examples of immune effector function, e.g., in a CART cell or CAR-expressing NK cell, include cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CAR-expressing immune effector cell, e.g., CART cell or CAR-expressing NK cell, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (“ICOS”), FcεRI, CD66d, DAP10, and DAP12.

The term “zeta” or alternatively “zeta chain”, “CD3-zeta” or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO:9. In one aspect, the “zeta stimulatory domain” or a “CD3-zeta stimulatory domain” is the sequence provided as SEQ ID NO:10.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an a MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to the intracellular portion of a costimulatory molecule. The intracellular signaling domain can comprise the entire intracellular portion, or the entire native intracellular signaling domain, of the molecule from which it is derived, or a functional fragment thereof.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank accno. AAA62478.2, or the equivalent residues from a non-human species, e.g., mouse, rodent, monkey, ape and the like. In one aspect, the “4-1BB costimulatory domain” is the sequence provided as SEQ ID NO:7 or the equivalent residues from a non-human species, e.g., mouse, rodent, monkey, ape and the like.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “vector” as used herein refers to any vehicle that can be used to deliver and/or express a nucleic acid molecule. It can be a transfer vector or an expression vector as described herein.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid,” “polynucleotide,” or “nucleic acid molecule” refers to deoxyribonucleic acid (DNA) or ribonucleic acid (RNA), or a combination of a DNA or RNA thereof, and polymers thereof in either single- or double-stranded form. The term “nucleic acid” includes a gene, cDNA or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The term “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 or CD123 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some enbodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n (SEQ ID NO: 38), where n is a positive integer equal to or greater than 1. For example, n=1, n=2, n=3. n=4, n=5 and n=6, n=7, n=8, n=9 and n=10 In one embodiment, the flexible polypeptide linkers include, but are not limited to, (Gly4 Ser)4 (SEQ ID NO:27) or (Gly4 Ser)3 (SEQ ID NO:28). In another embodiment, the linkers include multiple repeats of (Gly2Ser), (GlySer) or (Gly3Ser) (SEQ ID NO:29). Also included within the scope of the invention are linkers described in WO2012/138475, incorporated herein by reference).

As used herein, a 5′ cap (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, preferably mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In the preferred embodiment of a construct for transient expression, the polyA is between 50 and 5000 (SEQ ID NO: 30), preferably greater than 64, more preferably greater than 100, most preferably greater than 300 or 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

A dosage regimen, e.g., a therapeutic dosage regimen, can include one or more treatment intervals. The dosage regimen can result in at least one beneficial or desired clinical result including, but are not limited to, alleviation of a symptom, diminishment of extent of disease, stabilized (i.e., not worsening) state of disease, delay or slowing of disease progression, amelioration or palliation of the disease state, whether detectable or undetectable.

As used herein, a “treatment interval” refers to a treatment cycle, for example, a course of administration of a therapeutic agent that can be repeated, e.g., on a regular schedule. In embodiments, a dosage regimen can have one or more periods of no administration of the therapeutic agent in between treatment intervals. For example, a treatment interval can include one dose of a CAR molecule administered in combination with (prior, concurrently or after) administration of a second therapeutic agent, e.g., an inhibitor (e.g., a kinase inhibitor as described herein).

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

The term “therapeutic” as used herein means a treatment. A therapeutic effect is obtained by reduction, suppression, remission, or eradication of a disease state.

In embodiments, a disease state treated includes CRS. In some embodiments, treatment of CRS includes administration of a composition or combination described herein after the onset, e.g., after detection of, one or more CRS symptoms. In some embodiments, treatment of CRS results in a reduction in the severity of CRS, e.g., relative to a subject not administered the composition or combination described herein. For example, the subject may reduce CRS to an undetectable level. In other embodiments, the treatment results in a less severe form of CRS, e.g., grade 1, 2, or 3 CRS.

The term “prophylaxis” as used herein means the prevention of or protective treatment for a disease or disease state. Prevention of a disease or disease state can include reduction (e.g., mitigation) of one or more symptoms of the disease or disease state, e.g., relative to a reference level (e.g., the symptom(s) in a similar subject not administered the treatment). Prevention can also include delaying onset of one or more symptoms of the disease or disease state, e.g., relative to a reference level (e.g., the onset of the symptom(s) in a similar subject not administered the treatment). In embodiments, a disease is a disease described herein.

In embodiments, a disease state prevented includes CRS. In some embodiments, prevention of CRS includes administration of a composition or combination described herein prior to, e.g., prior to detection or onset of, one or more CRS symptoms. In some embodiments, administration of the JAK-STAT inhibitor or the BTK inhibitor occurs prior to the CAR therapy. In some embodiments, prevention of CRS results in a reduction in the likelihood or severity of CRS, e.g., relative to a subject not administered the composition or combination described herein. For example, the subject may not develop CRS. In other embodiments, the subject develops a less severe form of CRS, e.g., grade 1, 2, or 3 CRS, e.g., relative to a subject not administered the composition or combination described herein.

In the context of the present invention, “tumor antigen” or “hyperproliferative disorder antigen” or “antigen associated with a hyperproliferative disorder” refers to antigens that are common to specific hyperproliferative disorders. In certain aspects, the hyperproliferative disorder antigens of the present invention are derived from, cancers including but not limited to primary or metastatic melanoma, thymoma, lymphoma, sarcoma, lung cancer, liver cancer, non-Hodgkin lymphoma, non-Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with regulatable intracellular signal generation. In some embodiments, an RCAR comprises at least an extracellular antigen binding domain, a transmembrane and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, e.g., are in different polypeptide chains. In some embodiments, the RCAR includes a dimerization switch that, upon the presence of a dimerization molecule, can couple the polypeptides to one another, e.g., can couple an antigen binding domain to an intracellular signaling domain. In some embodiments, the RCAR is expressed in a cell (e.g., an immune effector cell) as described herein, e.g., an RCAR-expressing cell (also referred to herein as “RCARX cell”). In an embodiment the RCARX cell is a T cell, and is referred to as a RCART cell. In an embodiment the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, e.g., a myristoyl group, sufficient to anchor an extracellular or intracellular domain to the plasma membrane.

“Switch domain,” as that term is used herein, e.g., when referring to an RCAR, refers to an entity, typically a polypeptide-based entity, that, in the presence of a dimerization molecule, associates with another switch domain. The association results in a functional coupling of a first entity linked to, e.g., fused to, a first switch domain, and a second entity linked to, e.g., fused to, a second switch domain. A first and second switch domain are collectively referred to as a dimerization switch. In embodiments, the first and second switch domains are the same as one another, e.g., they are polypeptides having the same primary amino acid sequence, and are referred to collectively as a homodimerization switch. In embodiments, the first and second switch domains are different from one another, e.g., they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, e.g., FKBP or FRB-based, and the dimerization molecule is small molecule, e.g., a rapalogue. In embodiments, the switch domain is a polypeptide-based entity, e.g., an scFv that binds a myc peptide, and the dimerization molecule is a polypeptide, a fragment thereof, or a multimer of a polypeptide, e.g., a myc ligand or multimers of a myc ligand that bind to one or more myc scFvs. In embodiments, the switch domain is a polypeptide-based entity, e.g., myc receptor, and the dimerization molecule is an antibody or fragments thereof, e.g., myc antibody.

“Dimerization molecule,” as that term is used herein, e.g., when referring to an RCAR, refers to a molecule that promotes the association of a first switch domain with a second switch domain. In embodiments, the dimerization molecule does not naturally occur in the subject, or does not occur in concentrations that would result in significant dimerization. In embodiments, the dimerization molecule is a small molecule, e.g., rapamycin or a rapalogue, e.g, RAD001.

The term “bioequivalent” refers to an amount of an agent other than the reference compound (e.g., RAD001), required to produce an effect equivalent to the effect produced by the reference dose or reference amount of the reference compound (e.g., RAD001). In an embodiment the effect is the level of mTOR inhibition, e.g., as measured by P70 S6 kinase inhibition, e.g., as evaluated in an in vivo or in vitro assay, e.g., as measured by an assay described herein, e.g., the Boulay assay, or measurement of phosphorylated S6 levels by western blot. In an embodiment, the effect is alteration of the ratio of PD-1 positive/PD-1 negative immune effector cells, e.g., T cells or NK cells, as measured by cell sorting. In an embodiment a bioequivalent amount or dose of an mTOR inhibitor is the amount or dose that achieves the same level of P70 S6 kinase inhibition as does the reference dose or reference amount of a reference compound. In an embodiment, a bioequivalent amount or dose of an mTOR inhibitor is the amount or dose that achieves the same level of alteration in the ratio of PD-1 positive/PD-1 negative immune effector cells, e.g., T cells or NK cells as does the reference dose or reference amount of a reference compound.

The term “low, immune enhancing, dose” when used in conjuction with an mTOR inhibitor, e.g., an allosteric mTOR inhibitor, e.g., RAD001 or rapamycin, or a catalytic mTOR inhibitor, refers to a dose of mTOR inhibitor that partially, but not fully, inhibits mTOR activity, e.g., as measured by the inhibition of P70 S6 kinase activity. Methods for evaluating mTOR activity, e.g., by inhibition of P70 S6 kinase, are discussed herein. The dose is insufficient to result in complete immune suppression but is sufficient to enhance the immune response. In an embodiment, the low, immune enhancing, dose of mTOR inhibitor results in a decrease in the number of PD-1 positive immune effector cells, e.g., T cells or NK cells, and/or an increase in the number of PD-1 negative immune effector cells, e.g., T cells or NK cells, or an increase in the ratio of PD-1 negative T cells/PD-1 positive immune effector cells, e.g., T cells or NK cells.

In an embodiment, the low, immune enhancing, dose of mTOR inhibitor results in an increase in the number of naive immune effector cells, e.g., T cells or NK cells. In an embodiment, the low, immune enhancing, dose of mTOR inhibitor results in one or more of the following:
an increase in the expression of one or more of the following markers: CD62L^{high}, CD127^{high}, CD27⁺, and BCL2, e.g., on memory T cells, e.g., memory T cell precursors;
a decrease in the expression of KLRG1, e.g., on memory T cells, e.g., memory T cell precursors; and
an increase in the number of memory T cell precursors, e.g., cells with any one or combination of the following characteristics: increased CD62L^{high}, increased CD127^{high}, increased CD27⁺, decreased KLRG1, and increased BCL2;

wherein any of the changes described above occurs, e.g., at least transiently, e.g., as compared to a non-treated subject.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or “relapse” as used herein refers to the return or reappearance of a disease (e.g., cancer) or the signs and symptoms of a disease such as cancer after a period of improvement or responsiveness, e.g., after prior treatment of a therapy, e.g., cancer therapy. The initial period of responsiveness may involve the level of cancer cells falling below a certain threshold, e.g., below 20%, 1%, 10%, 5%, 4%, 3%, 2%, or 1%. The reappearance may involve the level of cancer cells rising above a certain threshold, e.g., above 20%, 1%, 10%, 5%, 4%, 3%, 2%, or 1%. For example, e.g., in the context of B-ALL, the reappearance may involve, e.g., a reappearance of blasts in the blood, bone marrow (>5%), or any extramedullary site, after a complete response. A complete response, in this context, may involve <5% BM blast. More generally, in an embodiment, a response (e.g., complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

In some embodiments, a therapy that includes a CD19 inhibitor, e.g., a CD19 CAR therapy, may relapse or be refractory to treatment. The relapse or resistance can be caused by CD19 loss (e.g., an antigen loss mutation) or other CD19 alteration that reduces the level of CD19 (e.g., caused by clonal selection of CD19-negative clones). A cancer that harbors such CD19 loss or alteration is referred to herein as a “CD19-negative cancer” or a “CD19-negative relapsed cancer”). It shall be understood that a CD19-negative cancer need not have 100% loss of CD19, but a sufficient reduction to reduce the effectiveness of a CD19 therapy such that the cancer relapses or becomes refractory. In some embodiments, a CD19-negative cancer results from a CD19 CAR therapy.

As used herein, “JAK-STAT” refers to the JAK-STAT signaling pathway and/or one or more kinase in the JAK-STAT pathway. The JAK-STAT signaling pathway and its components are described in greater detail herein.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

### Description

Provided herein are methods for preventing CRS in a subject. The method can include administration of a CAR described herein in combination with a kinase inhibitor, e.g., inhibitor of JAK-STAT or BTK.

Also provided herein are compositions of matter and methods of use for the treatment or prevention of a disease such as cancer using a chimeric antigen receptor (CAR) in combination with a kinase inhibitor, e.g., inhibitor of JAK-STAT or BTK.

Example 3 herein describes that in CAR T cell-associated CRS, IL-6 is produced by antigen presenting cells (myeloid cells) and that IL-6 presence or absence (e.g., as measured by degranulation in the presence or absence of APCs) did not affect CART function. Accordingly, in some embodiments, a CAR described herein is administered in combination with an IL-6 inhibitor, e.g., tocilizumab. In embodiments, methods described herein provide for early administration of an IL-6 inhibitor, e.g., tocilizumab, to prevent CRS associated with CAR therapy. In embodiments, early administration include administration prior to a CAR therapy, at the same time as a CAR therapy dose, or up until a first sign of a fever (e.g., after a CAR therapy dose). In some embodiments, the combination of CAR and IL-6 inhibitor described herein can further comprise a kinase inhibitor, e.g., a kinase inhibitor as described herein.

A chimeric antigen receptor (CAR) comprising an antibody or antibody fragment engineered for specific binding to an antigen (e.g., CD123 protein or CD19 protein or fragments thereof) can be used in accordance with any method or composition described herein. In one aspect, the invention provides a cell (e.g., an immune effector cell, e.g., a T cell or a NK cell) engineered to express a CAR, wherein the CAR-expressing cell (e.g., “CART” or CAR-expressing NK cell) exhibits an antitumor property. In one aspect a cell is transformed with the CAR and the at least part of the CAR is expressed on the cell surface. In some embodiments, the cell (e.g., immune effector cell, e.g., T cell or NK cell) is transduced with a viral vector encoding a CAR. In some embodiments, the viral vector is a retroviral vector. In some embodiments, the viral vector is a lentiviral vector. In some such embodiments, the cell may stably express the CAR. In another embodiment, the cell (e.g., immune effector cell, e.g., T cell or NK cell) is transfected with a nucleic acid, e.g., mRNA, cDNA, DNA, encoding a CAR. In some such embodiments, the cell may transiently express the CAR.

In one aspect, the antigen binding domain (e.g., CD123 binding domain or CD19 binding domain), e.g., the human or humanized CD123 binding domain or CD19 binding domain, of the CAR is a scFv antibody fragment. In one aspect, such antibody fragments are functional in that they retain the equivalent binding affinity, e.g., they bind the same antigen with comparable efficacy, as the IgG antibody having the same heavy and light chain variable regions. In one aspect such antibody fragments are functional in that they provide a biological response that can include, but is not limited to, activation of an immune response, inhibition of signal-transduction origination from its target antigen, inhibition of kinase activity, and the like, as will be understood by a skilled artisan.

In some aspects, the antibodies of the invention are incorporated into a chimeric antigen receptor (CAR). In one aspect, the CAR is a CD123 CAR and comprises the polypeptide sequence provided herein as SEQ ID NOS: 98-101, and 125-156.

In one aspect, the antigen binding domain (CD123 or CD19 binding domain, e.g., humanized or human CD123 or CD19 binding domain) portion of a CAR of the invention is encoded by a transgene whose sequence has been codon optimized for expression in a mammalian cell. In one aspect, entire CAR construct of the invention is encoded by a transgene whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

In one aspect, the antigen binding domain of the CAR comprises a human CD123 antibody or antibody fragment or a human CD19 antibody or antibody fragment. In one aspect, the antigen binding domain of the CAR comprises a humanized CD123 or CD19 antibody or antibody fragment. In one aspect, the antigen binding domain of the CAR comprises human CD123 or CD19 antibody fragment comprising an scFv. In one aspect, the antigen binding domain of the CAR is a human CD123 scFv or a human CD19 scFv. In one aspect, the antigen binding domain of the CAR comprises a humanized CD123 or CD19 antibody fragment comprising an scFv. In one aspect, the antigen binding domain of the CAR is a humanized CD123 scFv or CD19 scFv.

In one aspect, the CAR123 binding domain comprises the scFv portion provided in SEQ ID NO:157-160 and 184-215. In one aspect the scFv portion is human. In one aspect, the human CAR123 binding domain comprises the scFv portion provided in SEQ ID NO:157-160. In one aspect, the human CD123 binding domain comprises the scFv portion provided in SEQ ID NO: 478, 480, 483, or 485.

In one aspect the scFv portion is humanized. In one aspect, the humanized CAR123 binding domain comprises the scFv portion provided in SEQ ID NO:184-215. In one aspect, the humanized CD123 binding domain comprises the scFv portion provided in SEQ ID NOs: 556-587.

Furthermore, the present invention provides CD123 CAR compositions and their use in medicaments or methods for treating, among other diseases, cancer or any malignancy or autoimmune diseases involving cells or tissues which express CD123.

In one aspect, the CAR of the invention can be used to eradicate CD123-expressing normal cells, thereby applicable for use as a cellular conditioning therapy prior to cell transplantation. In one aspect, the CD123-expressing normal cell is a CD123-expressing expressing myeloid progenitor cell and the cell transplantation is a stem cell transplantation.

In one aspect, the invention provides a cell (e.g., an immune effector cell, e.g., a T cell or NK cell) engineered to express a chimeric antigen receptor (e.g., CAR-expressing immune effector cell, e.g., CART or CAR-expressing NK cell) of the present invention, wherein the cell (e.g., “CART”) exhibits an antitumor property. Accordingly, the invention provides a CD123-CAR that comprises a CD123 binding domain and is engineered into an immune effector cell, e.g., a T cell or a NK cell, and methods of their use for adoptive therapy.

In one aspect, the CD123-CAR comprises at least one intracellular domain, e.g., described herein, e.g., selected from the group of a CD137 (4-1BB) signaling domain, a CD28 signaling domain, a CD3zeta signal domain, and any combination thereof. In one aspect, the CD123-CAR comprises at least one intracellular signaling domain is from one or more co-stimulatory molecule(s) other than a CD137 (4-1BB) or CD28.

### Chimeric Antigen Receptor (CAR)

In accordance with any method or composition described herein, in embodiments, a CAR molecule comprises a CD123 CAR described herein, e.g., a CD123 CAR described in US2014/0322212A1 or US2016/0068601A1, both incorporated herein by reference. In embodiments, the CD123 CAR comprises an amino acid, or has a nucleotide sequence shown in US2014/0322212A1 or US2016/0068601A1, both incorporated herein by reference. In other embodiments, a CAR molecule comprises a CD19 CAR molecule described herein, e.g., a CD19 CAR molecule described in US-2015-0283178-A1, e.g., CTL019. In embodiments, the CD19 CAR comprises an amino acid, or has a nucleotide sequence shown in US-2015-0283178-A1, incorporated herein by reference. In one embodiment, CAR molecule comprises a BCMA CAR molecule described herein, e.g., a BCMA CAR described in US-2016-0046724-A1. In embodiments, the BCMA CAR comprises an amino acid, or has a nucleotide sequence shown in US-2016-0046724-A1, incorporated herein by reference. In an embodiment, the CAR molecule comprises a CLL1 CAR described herein, e.g., a CLL1 CAR described in US2016/0051651A1, incorporated herein by reference. In embodiments, the CLL1 CAR comprises an amino acid, or has a nucleotide sequence shown in US2016/0051651A1, incorporated herein by reference. In an embodiment, the CAR molecule comprises a CD33 CAR described herein, e.ga CD33 CAR described in US2016/0096892A1, incorporated herein by reference. In embodiments, the CD33 CAR comprises an amino acid, or has a nucleotide sequence shown in US2016/0096892A1, incorporated herein by reference. In an embodiment, the CAR molecule comprises an EGFRvIII CAR molecule described herein, e.g., an EGFRvIII CAR described US2014/0322275A1, incorporated herein by reference. In embodiments, the EGFRvIII CAR comprises an amino acid, or has a nucleotide sequence shown in US2014/0322275A1, incorporated herein by reference. In an embodiment, the CAR molecule comprises a mesothelin CAR described herein, e.g., a mesothelin CAR described in WO 2015/090230, incorporated herein by reference. In embodiments, the mesothelin CAR comprises an amino acid, or has a nucleotide sequence shown in WO 2015/090230, incorporated herein by reference.

### CAR123

The present invention encompasses a recombinant DNA construct comprising sequences encoding a CAR, wherein the CAR comprises an antigen binding domain (e.g., antibody, antibody fragment) that binds specifically to CD123 or a fragment thereof, e.g., human CD123, wherein the sequence of the CD123 binding domain (e.g., antibody or antibody fragment) is, e.g., contiguous with and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

In specific aspects, a CAR construct of the invention comprises a scFv domain selected from the group consisting of SEQ ID NOS:157-160, 184-215, 478, 480, 483, 485, and 556-587 wherein the scFv may be preceded by an optional leader sequence such as provided in SEQ ID NO: 1, and followed by an optional hinge sequence such as provided in SEQ ID NO:2 or SEQ ID NO:3 or SEQ ID NO:4 or SEQ ID NO:5, a transmembrane region such as provided in SEQ ID NO:6, an intracellular signalling domain that includes SEQ ID NO:7 or SEQ ID NO:8 and a CD3 zeta sequence that includes SEQ ID NO:9 or SEQ ID NO:10, e.g., wherein the domains are contiguous with and in the same reading frame to form a single fusion protein. In some embodiments, the scFv domain is a human scFv domain selected from the group consisting of SEQ ID NOS: 157-160, 478, 480, 483, and 485. In some embodiments, the scFv domain is a humanized scFv domain selected from the group consisting of SEQ ID NOS: 184-215 and 556-587. Also included in the invention is a nucleotide sequence that encodes the polypeptide of each of the scFv fragments selected from the group consisting of SEQ ID NO: 157-160, 184-215, 478, 480, 483, 485, and 556-587. Also included in the invention is a nucleotide sequence that encodes the polypeptide of each of the scFv fragments selected from the group consisting of SEQ ID NO: 157-160, 184-215, 478, 480, 483, 485, and 556-587, and each of the domains of SEQ ID NOS: 1, 2, and 6-9, plus the encoded CD123 CAR of the invention.

In one aspect an exemplary CD123CAR constructs comprise an optional leader sequence, an extracellular antigen binding domain, a hinge, a transmembrane domain, and an intracellular stimulatory domain. In one aspect an exemplary CD123CAR construct comprises an optional leader sequence, an extracellular antigen binding domain, a hinge, a transmembrane domain, an intracellular costimulatory domain and an intracellular stimulatory domain.

In some embodiments, full-length CD123 CAR sequences are also provided herein as SEQ ID NOS: 98-101 and 125-156, as shown in Table 11A or 12A.

An exemplary leader sequence is provided as SEQ ID NO: 1. An exemplary hinge/spacer sequence is provided as SEQ ID NO:2 or SEQ ID NO:3 or SEQ ID NO:4 or SEQ ID NO:5. An exemplary transmembrane domain sequence is provided as SEQ ID NO:6. An exemplary sequence of the intracellular signaling domain of the 4-1BB protein is provided as SEQ ID NO: 7. An exemplary sequence of the intracellular signaling domain of CD27 is provided as SEQ ID NO:8. An exemplary CD3zeta domain sequence is provided as SEQ ID NO: 9 or SEQ ID NO:10. An exemplary sequence of the intracellular signaling domain of CD28 is provided as SEQ ID NO:43. An exemplary sequence of the intracellular signaling domain of ICOS is provided as SEQ ID NO:45.

In one aspect, the present invention encompasses a recombinant nucleic acid construct comprising a nucleic acid molecule encoding a CAR, wherein the nucleic acid molecule comprises the nucleic acid sequence encoding a CD123 binding domain, e.g., described herein, e.g., that is contiguous with and in the same reading frame as a nucleic acid sequence encoding an intracellular signaling domain. In one aspect, a CD123 binding domain is selected from one or more of SEQ ID NOS: 157-160, 184-215, 478, 480, 483, 485, and 556-587. In some embodiments, the CD123 binding domain is a human CD123 binding domain selected from the group consisting of SEQ ID NOS: 157-160, 478, 480, 483, and 485. In some embodiments, the CD123 binding domain is a humanized CD123 binding domain selected from the group consisting of SEQ ID NOS: 184-215 and 556-587.

In one aspect, the present invention encompasses a recombinant nucleic acid construct comprising a nucleic acid molecule encoding a CAR, wherein the nucleic acid molecule comprises a nucleic acid sequence encoding a CD123 binding domain, e.g., wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, e.g., CD3-zeta, CD28, 4-1BB, ICOS, and the like. In some instances, the CAR can comprise any combination of CD3-zeta, CD28, 4-1BB, ICOS, and the like.

In one aspect, the nucleic acid sequence of a CAR construct of the invention is selected from one or more of SEQ ID NOS:39-42 and 66-97. The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the nucleic acid of interest can be produced synthetically, rather than cloned.

### CAR19 (or CD19 CAR)

The present disclosure encompasses immune effector cells (e.g., T cells or NK cells) comprising a CAR molecule that targets, e.g., specifically binds, to CD19 (CD19 CAR). In one embodiment, the immune effector cells are engineered to express the CD19 CAR. In one embodiment, the immune effector cells comprise a recombinant nucleic acid construct comprising nucleic acid sequences encoding the CD19 CAR.

In embodiments, the CD19 CAR comprises an antigen binding domain that specifically binds to CD19, e.g., CD19 binding domain, a transmembrane domain, and an intracellular signaling domain. In one embodiment, the sequence of the antigen binding domain is contiguous with and in the same reading frame as a nucleic acid sequence encoding an intracellular signaling domain. The intracellular signaling domain can comprise a costimulatory signaling domain and/or a primary signaling domain, e.g., a zeta chain. The costimulatory signaling domain refers to a portion of the CAR comprising at least a portion of the intracellular domain of a costimulatory molecule.

In one aspect, exemplary CAR constructs comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

In one aspect, the CD19 CARs of the invention comprise at least one intracellular signaling domain selected from the group of a CD137 (4-1BB) signaling domain, a CD28 signaling domain, a CD27 signaling domain, an ICOS signaling domain, a CD3zeta signal domain, and any combination thereof. In one aspect, the CARs of the invention comprise at least one intracellular signaling domain is from one or more costimulatory molecule(s) selected from CD137 (4-1BB), CD28, CD27, or ICOS.

### Vectors and RNA Constructs

The present invention includes retroviral and lentiviral vector constructs expressing a CAR that can be directly transduced into a cell.

The present invention also includes an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO:35). RNA so produced can efficiently transfect different kinds of cells. In one embodiment, the template includes sequences for the CAR. In an embodiment, an RNA CAR vector is transduced into a T cell by electroporation.

### Antigen Binding Domain

In one aspect, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the invention include those associated with viral, bacterial and parasitic infections, autoimmune disease and cancer cells.

In one aspect, the CAR-mediated T-cell response can be directed to an antigen of interest by way of engineering an antigen binding domain that specifically binds a desired antigen into the CAR.

In one aspect, the portion of the CAR comprising the antigen binding domain comprises an antigen binding domain that targets a tumor antigen, e.g., a tumor antigen described herein.

In one aspect, the portion of the CAR comprising the antigen binding domain comprises an antigen binding domain that targets CD123 or a fragment thereof. In embodiments, the antigen binding domain targets human CD123 or a fragment thereof. In other embodiments, the antigen binding domain targets a B cell antigen (e.g., B cell surface antigen), e.g., CD10, CD19, CD20, CD22, CD34, CD123, FLT-3, ROR1, CD79b, CD179b, or CD79a.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

In one embodiment, the antigen binding domain comprises one, two three (e.g., all three) heavy chain CDRs, HC CDR1, HC CDR2 and HC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (e.g., all three) light chain CDRs, LC CDR1, LC CDR2 and LC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In one embodiment, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

In embodiments, the antigen binding domain targets BCMA and is described in US-2016-0046724-A1.

In embodiments, the antigen binding domain targets CD19 and is described in US-2015-0283178-A1.

In embodiments, the antigen binding domain targets CD123 and is described in US2014/0322212A1, US2016/0068601A1.

In embodiments, the antigen binding domain targets CLL and is described in US2016/0051651A1.

In embodiments, the antigen binding domain targets CD33 and is described in US2016/0096892A1.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In other embodiments, the CAR-expressing cells can specifically bind to humanized CD19, e.g., can include a CAR molecule, or an antigen binding domain (e.g., a humanized antigen binding domain) according to Table 3 of WO2014/153270, incorporated herein by reference. The amino acid and nucleotide sequences encoding the CD19 CAR molecules and antigen binding domains (e.g., including one, two, three VH CDRs; and one, two, three VL CDRs according to Kabat or Chothia), are specified in WO2014/153270.

In other embodiments, the CAR-expressing cells can specifically bind to CD123, e.g., can include a CAR molecule (e.g., any of the CAR1 to CAR8), or an antigen binding domain according to Tables 1-2 of WO 2014/130635, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO 2014/130635.

In other embodiments, the CAR-expressing cells can specifically bind to CD123, e.g., can include a CAR molecule (e.g., any of the CAR123-1 ro CAR123-4 and hzCAR123-1 to hzCAR123-32), or an antigen binding domain according to Tables 2, 6, and 9 of WO2016/028896, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO2016/028896.

In other embodiments, the CAR-expressing cells can specifically bind to EGFRvIII, e.g., can include a CAR molecule, or an antigen binding domain according to Table 2 or SEQ ID NO:11 of WO 2014/130657, incorporated herein by reference. The amino acid and nucleotide sequences encoding the EGFRvIII CAR molecules and antigen binding domains (e.g., including one, two, three VH CDRs; and one, two, three VL CDRs according to Kabat or Chothia), are specified in WO 2014/130657.

In other embodiments, the CAR-expressing cells can specifically bind to CD33, e.g., can include a CAR molecule (e.g., any of CAR33-1 to CAR-33-9), or an antigen binding domain according to Table 2 or 9 of WO2016/014576, incorporated herein by reference. The amino acid and nucleotide sequences encoding the CD33 CAR molecules and antigen binding domains (e.g., including one, two, three VH CDRs; and one, two, three VL CDRs according to Kabat or Chothia), are specified in WO2016/014576.

In other embodiments, the CAR-expressing cells can specifically bind to mesothelin, e.g., can include a CAR molecule, or an antigen binding domain according to Tables 2-3 of WO 2015/090230, incorporated herein by reference. The amino acid and nucleotide sequences encoding the mesothelin CAR molecules and antigen binding domains (e.g., including one, two, three VH CDRs; and one, two, three VL CDRs according to Kabat or Chothia), are specified in WO 2015/090230.

In other embodiments, the CAR-expressing cells can specifically bind to BCMA, e.g., can include a CAR molecule, or an antigen binding domain according to Table 1 or 16, SEQ ID NO: 271 or SEQ ID NO: 273 of WO2016/014565, incorporated herein by reference. The amino acid and nucleotide sequences encoding the BCMA CAR molecules and antigen binding domains (e.g., including one, two, three VH CDRs; and one, two, three VL CDRs according to Kabat or Chothia), are specified in WO2016/014565.

In other embodiments, the CAR-expressing cells can specifically bind to CLL-1, e.g., can include a CAR molecule, or an antigen binding domain according to Table 2 of WO2016/014535, incorporated herein by reference. The amino acid and nucleotide sequences encoding the CLL-1 CAR molecules and antigen binding domains (e.g., including one, two, three VH CDRs; and one, two, three VL CDRs according to Kabat or Chothia), are specified in WO2016/014535.

In other embodiments, the CAR-expressing cells can specifically bind to GFR ALPHA-4, e.g., can include a CAR molecule, or an antigen binding domain according to Table 2 of WO2016/025880, incorporated herein by reference. The amino acid and nucleotide sequences encoding the GFR ALPHA-4 CAR molecules and antigen binding domains (e.g., including one, two, three VH CDRs; and one, two, three VL CDRs according to Kabat or Chothia), are specified in WO2016/025880.

In one embodiment, the antigen binding domain of any of the CAR molecules described herein (e.g., any of CD19, CD123, EGFRvIII, CD33, mesothelin, BCMA, and GFR ALPHA-4) comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antigen binding domain listed above. In one embodiment, the antigen binding domain comprises a heavy chain variable region and/or a variable light chain region of an antibody listed or described above.

In another aspect, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In one aspect, the antigen binding domain is humanized.

In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. Thus, in one aspect, the antigen binding domain comprises a human antibody or an antibody fragment.

CD123 Binding Domain

In one embodiment, the human CD123 binding domain comprises one or more (e.g., all three) light chain complementary determining region 1 (LC CDR1), light chain complementary determining region 2 (LC CDR2), and light chain complementary determining region 3 (LC CDR3) of a human CD123 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a human CD123 binding domain described herein, e.g., a human CD123 binding domain comprising one or more, e.g., all three, LC CDRs and one or more, e.g., all three, HC CDRs. In one embodiment, the human CD123 binding domain comprises one or more (e.g., all three) heavy chain complementary determining region 1 (HC CDR1), heavy chain complementary determining region 2 (HC CDR2), and heavy chain complementary determining region 3 (HC CDR3) of a human CD123 binding domain described herein, e.g., the human CD123 binding domain has two variable heavy chain regions, each comprising a HC CDR1, a HC CDR2 and a HC CDR3 described herein. In one embodiment, the human CD123 binding domain comprises a human light chain variable region described herein (e.g., in Table 11A or 12B) and/or a human heavy chain variable region described herein (e.g., in 11A or 12B). In one embodiment, the human CD123 binding domain comprises a human heavy chain variable region described herein (e.g., in Table 11A or 12B 9), e.g., at least two human heavy chain variable regions described herein (e.g., in Table 11A or 12B). In one embodiment, the CD123 binding domain is a scFv comprising a light chain and a heavy chain of an amino acid sequence of Table 11A or 12B. In an embodiment, the CD123 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 11A or 12B, or a sequence with at least 95% identity, e.g., 95-99% identity, with an amino acid sequence of Table 11A; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 11A or 12B, or a sequence with at least 95% identity, e.g., 95-99% identity, to an amino acid sequence of Table 11A or 12B. In one embodiment, the human CD123 binding domain comprises a sequence selected from a group consisting of SEQ ID NO:157-160, 478, 480, 483, and 485, or a sequence with at least 95% identity, e.g., 95-99% identity, thereof. In one embodiment, the human CD123 binding domain is a scFv, and a light chain variable region comprising an amino acid sequence described herein, e.g., in Table 11A or 12B, is attached to a heavy chain variable region comprising an amino acid sequence described herein, e.g., in Table 11A, via a linker, e.g., a linker described herein. In one embodiment, the human CD123 binding domain includes a (Gly₄-Ser)n linker, wherein n is 1, 2, 3, 4, 5, or 6, preferably 3 or 4 (SEQ ID NO:26). The light chain variable region and heavy chain variable region of a scFv can be, e.g., in any of the following orientations: light chain variable region-linker-heavy chain variable region or heavy chain variable region-linker-light chain variable region.

In some aspects, a non-human antibody is humanized, where specific sequences or regions of the antibody are modified to increase similarity to an antibody naturally produced in a human or fragment thereof. Thus, in one aspect, the antigen binding domain comprises a humanized antibody or an antibody fragment. In one embodiment, the humanized CD123 binding domain comprises one or more (e.g., all three) light chain complementary determining region 1 (LC CDR1), light chain complementary determining region 2 (LC CDR2), and light chain complementary determining region 3 (LC CDR3) of a humanized CD123 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized CD123 binding domain described herein, e.g., a humanized CD123 binding domain comprising one or more, e.g., all three, LC CDRs and one or more, e.g., all three, HC CDRs. In one embodiment, the humanized CD123 binding domain comprises one or more (e.g., all three) heavy chain complementary determining region 1 (HC CDR1), heavy chain complementary determining region 2 (HC CDR2), and heavy chain complementary determining region 3 (HC CDR3) of a humanized CD123 binding domain described herein, e.g., the humanized CD123 binding domain has two variable heavy chain regions, each comprising a HC CDR1, a HC CDR2 and a HC CDR3 described herein. In one embodiment, the humanized CD123 binding domain comprises a humanized light chain variable region described herein (e.g., in Table 12A) and/or a humanized heavy chain variable region described herein (e.g., in Table 12A). In one embodiment, the humanized CD123 binding domain comprises a humanized heavy chain variable region described herein (e.g., in Table 12A), e.g., at least two humanized heavy chain variable regions described herein (e.g., in Table 12A). In one embodiment, the CD123 binding domain is a scFv comprising a light chain and a heavy chain of an amino acid sequence of Table 12A. In an embodiment, the CD123 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 4, or a sequence with at least 95% identity, e.g., 95-99% identity, with an amino acid sequence of Table 12A; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 12A, or a sequence with at least 95% identity, e.g., 95-99% identity, to an amino acid sequence of Table 12A. In one embodiment, the humanized CD123 binding domain comprises a sequence selected from a group consisting of SEQ ID NO:184-215 and 302-333, or a sequence with at least 95% identity, e.g., 95-99% identity, thereof. In one embodiment, the humanized CD123 binding domain is a scFv, and a light chain variable region comprising an amino acid sequence described herein, e.g., in Table 12A, is attached to a heavy chain variable region comprising an amino acid sequence described herein, e.g., in Table 12A, via a linker, e.g., a linker described herein. In one embodiment, the humanized CD123 binding domain includes a (Gly4-Ser)n linker, wherein n is 1, 2, 3, 4, 5, or 6, preferably 3 or 4 (SEQ ID NO:26). The light chain variable region and heavy chain variable region of a scFv can be, e.g., in any of the following orientations: light chain variable region-linker-heavy chain variable region or heavy chain variable region-linker-light chain variable region.

### Humanized Antibody

A humanized antibody can be produced using a variety of techniques known in the art, including but not limited to, CDR-grafting (see, e.g., European Patent No. EP 239,400; International Publication No. WO 91/09967; and U.S. Pat. Nos. 5,225,539, 5,530,101, and 5,585,089, each of which is incorporated herein in its entirety by reference), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973, each of which is incorporated herein by its entirety by reference), chain shuffling (see, e.g., U.S. Pat. No. 5,565,332, which is incorporated herein in its entirety by reference), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994), each of which is incorporated herein in its entirety by reference. Often, framework residues in the framework regions will be substituted with the corresponding residue from the CDR donor antibody to alter, for example improve, antigen binding. These framework substitutions are identified by methods well-known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323, which are incorporated herein by reference in their entireties.)

A humanized antibody or antibody fragment has one or more amino acid residues remaining in it from a source which is nonhuman. These nonhuman amino acid residues are often referred to as “import” residues, which are typically taken from an “import” variable domain. As provided herein, humanized antibodies or antibody fragments comprise one or more CDRs from nonhuman immunoglobulin molecules and framework regions wherein the amino acid residues comprising the framework are derived completely or mostly from human germline. Multiple techniques for humanization of antibodies or antibody fragments are well-known in the art and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640, the contents of which are incorporated herein by reference herein in their entirety). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332), the contents of which are incorporated herein by reference herein in their entirety.

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987), the contents of which are incorporated herein by reference herein in their entirety). Another method uses a particular framework derived from the consensus sequence of all human antibodies of a particular subgroup of light or heavy chains. The same framework may be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993), the contents of which are incorporated herein by reference herein in their entirety). In some embodiments, the framework region, e.g., all four framework regions, of the heavy chain variable region are derived from a VH4_4-59 germline sequence. In one embodiment, the framework region can comprise, one, two, three, four or five modifications, e.g., substitutions, e.g., from the amino acid at the corresponding murine sequence. In one embodiment, the framework region, e.g., all four framework regions of the light chain variable region are derived from a VK3_1.25 germline sequence. In one embodiment, the framework region can comprise, one, two, three, four or five modifications, e.g., substitutions, e.g., from the amino acid at the corresponding murine sequence.

In some aspects, the portion of a CAR composition of the invention that comprises an antibody fragment is humanized with retention of high affinity for the target antigen and other favorable biological properties. According to one aspect of the invention, humanized antibodies and antibody fragments are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, e.g., the analysis of residues that influence the ability of the candidate immunoglobulin to bind the target antigen. In this way, FR residues can be selected and combined from the recipient and import sequences so that the desired antibody or antibody fragment characteristic, such as increased affinity for the target antigen, is achieved. In general, the CDR residues are directly and most substantially involved in influencing antigen binding.

A humanized antibody or antibody fragment may retain a similar antigenic specificity as the original antibody, e.g., in the present invention, the ability to bind an antigen described herein, e.g., tumor antigen, e.g., B cell antigen, e.g., human CD123, CD19, or a fragment thereof. In some embodiments, a humanized antibody or antibody fragment may have improved affinity and/or specificity of binding to the antigen, e.g., tumor antigen, e.g., B cell antigen, e.g., human CD123, CD19, or a fragment thereof.

In one aspect, the antigen binding domain portion comprises one or more sequence selected from SEQ ID NOS:157-160, 184-215, 478, 480, 483, 485, and 556-587. In one aspect, the CD123 CAR that includes a human CD123 binding domain is selected from one or more sequence selected from SEQ ID NOS:157-160, 478, 480, 483, and 485. In one aspect, the CD123 CAR that includes a humanized CD123 binding domain is selected from one or more sequence selected from SEQ ID NOS:184-215 and 556-587.

In one aspect, the antigen binding domain (e.g., tumor antigen binding domain, e.g., B cell antigen binding domain, e.g., CD123 binding domain or CD19 binding domain) is characterized by particular functional features or properties of an antibody or antibody fragment. For example, in one aspect, the portion of a CAR composition of the invention that comprises an antigen binding domain specifically binds the antigen (e.g., tumor antigen, e.g., B cell antigen, e.g., human CD123, CD19, or a fragment thereof). In one aspect, the invention relates to an antigen binding domain comprising an antibody or antibody fragment, wherein the antibody binding domain specifically binds to a CD123 protein or fragment thereof, wherein the antibody or antibody fragment comprises a variable light chain and/or a variable heavy chain that includes an amino acid sequence of SEQ ID NO: 157-160, 184-215, 478, 480, 483, 485, and 556-587. In one aspect, the antigen binding domain comprises an amino acid sequence of an scFv selected from SEQ ID NO: 157-160, 184-215, 478, 480, 483, 485, and 556-587. In certain aspects, the scFv is contiguous with and in the same reading frame as a leader sequence. In one aspect the leader sequence is the polypeptide sequence provided as SEQ ID NO:1.

### Antigen Binding Domain—Additional Embodiments

In one aspect, the antigen binding domain (e.g., tumor antigen binding domain, e.g., B cell antigen binding domain, e.g., CD123 binding domain or CD19 binding domain) is a fragment, e.g., a single chain variable fragment (scFv). In one aspect, the antigen binding domain (e.g., tumor antigen binding domain, e.g., B cell antigen binding domain, e.g., CD123 binding domain or CD19 binding domain) is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds an antigen (e.g., tumor antigen, e.g., B cell antigen, e.g., CD123 or CD19 protein) or fragment thereof with wild-type or enhanced affinity.

In some instances, a human scFv can be derived from a display library. A display library is a collection of entities; each entity includes an accessible polypeptide component and a recoverable component that encodes or identifies the polypeptide component. The polypeptide component is varied so that different amino acid sequences are represented. The polypeptide component can be of any length, e.g. from three amino acids to over 300 amino acids. A display library entity can include more than one polypeptide component, for example, the two polypeptide chains of a Fab. In one exemplary embodiment, a display library can be used to identify a human CD123 binding domain. In a selection, the polypeptide component of each member of the library is probed with CD123, or a fragment thereof, and if the polypeptide component binds to CD123, the display library member is identified, typically by retention on a support.

Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component, i.e., the anti-CD123 binding domain, and purification of the polypeptide component for detailed characterization.

A variety of formats can be used for display libraries. Examples include the phaage display. In phage display, the protein component is typically covalently linked to a bacteriophage coat protein. The linkage results from translation of a nucleic acid encoding the protein component fused to the coat protein. The linkage can include a flexible peptide linker, a protease site, or an amino acid incorporated as a result of suppression of a stop codon. Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8 and Hoet et al. (2005) Nat Biotechnol. 23(3)344-8. Bacteriophage displaying the protein component can be grown and harvested using standard phage preparatory methods, e.g. PEG precipitation from growth media. After selection of individual display phages, the nucleic acid encoding the selected protein components can be isolated from cells infected with the selected phages or from the phage themselves, after amplification. Individual colonies or plaques can be picked, the nucleic acid isolated and sequenced.

Other display formats include cell based display (see, e.g., WO 03/029456), protein-nucleic acid fusions (see, e.g., U.S. Pat. No. 6,207,446), ribosome display (See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30; and Schaffitzel et al. (1999) J Immunol Methods. 231(1-2):119-35), and E. coli periplasmic display (2005 Nov. 22; PMID: 16337958).

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO:25). In one embodiment, the linker can be (Gly₄Ser)₄ (SEQ ID NO:27) or (Gly₄Ser)₃(SEQ ID NO:28). Variation in the linker length may retain or enhance activity, giving rise to superior efficacy in activity studies.

### Exemplary CD123 CAR Constructs and Antigen Binding Domains

Exemplary CD123 CAR constructs disclose herein comprise an scFv (e.g., a human scFv as disclosed in Tables 11A, 12A and 12B herein, optionally preceded with an optional leader sequence (e.g., SEQ ID NO:1 and SEQ ID NO:12 for exemplary leader amino acid and nucleotide sequences, respectively). The sequences of the human scFv fragments (amino acid sequences of SEQ ID NOs:157-160) are provided herein in Table 11A. The sequences of human scFv fragments, without the leader sequence, are provided herein in Table 12B (SEQ ID NOs: 479, 481, 482, and 484 for the nucleotide sequences, and SEQ ID NOs: 478, 480, 483, and 485 for the amino acid sequences). The CD123 CAR construct can further include an optional hinge domain, e.g., a CD8 hinge domain (e.g., including the amino acid sequence of SEQ ID NO: 2 or encoded by a nucleic acid sequence of SEQ ID NO:13); a transmembrane domain, e.g., a CD8 transmembrane domain (e.g., including the amino acid sequence of SEQ ID NO: 6 or encoded by the nucleotide sequence of SEQ ID NO: 17); an intracellular domain, e.g., a 4-1BB intracellular domain (e.g., including the amino acid sequence of SEQ ID NO: 7 or encoded by the nucleotide sequence of SEQ ID NO: 18; and a functional signaling domain, e.g., a CD3 zeta domain (e.g., including amino acid sequence of SEQ ID NO: 9 or 10, or encoded by the nucleotide sequence of SEQ ID NO: 20 or 21). In certain embodiments, the domains are contiguous with and in the same reading frame to form a single fusion protein. In other embodiments, the domain are in separate polypeptides, e.g., as in an RCAR molecule as described herein.

In certain embodiments, the full length CD123 CAR molecule includes the amino acid sequence of, or is encoded by the nucleotide sequence of, CD123-1, CD123-2, CD123-3, CD123-4, hzCD123-1, hzCD123-2, hzCD123-3, hzCD123-4, hzCD123-5, hzCD123-6, hzCD123-7, hzCD123-8, hzCD123-9, hzCD123-10, hzCD123-11, hzCD123-12, hzCD123-13, hzCD123-14, hzCD123-15, hzCD123-16, hzCD123-17, hzCD123-18, hzCD123-19, hzCD123-20, hzCD123-21, hzCD123-22, hzCD123-23, hzCD123-24, hzCD123-25, hzCD123-26, hzCD123-27, hzCD123-28, hzCD123-29, hzCD123-30, hzCD123-31, or hzCD123-32, provided in Table 11A, 12A or 12B, or a sequence substantially identical (e.g., with at least 95% identity, e.g., 95-99% identity) thereto.

In certain embodiments, the CD123 CAR molecule, or the CD123 antigen binding domain, includes the scFv amino acid sequence of CD123-1, CD123-2, CD123-3, CD123-4, hzCD123-1, hzCD123-2, hzCD123-3, hzCD123-4, hzCD123-5, hzCD123-6, hzCD123-7, hzCD123-8, hzCD123-9, hzCD123-10, hzCD123-11, hzCD123-12, hzCD123-13, hzCD123-14, hzCD123-15, hzCD123-16, hzCD123-17, hzCD123-18, hzCD123-19, hzCD123-20, hzCD123-21, hzCD123-22, hzCD123-23, hzCD123-24, hzCD123-25, hzCD123-26, hzCD123-27, hzCD123-28, hzCD123-29, hzCD123-30, hzCD123-31, or hzCD123-32, provided in Table 11A, 12A or 12B; or includes the scFv amino acid sequence of, or is encoded by the nucleotide sequence of, CD123-1, CD123-2, CD123-3, CD123-4, hzCD123-1, hzCD123-2, hzCD123-3, hzCD123-4, hzCD123-5, hzCD123-6, hzCD123-7, hzCD123-8, hzCD123-9, hzCD123-10, hzCD123-11, hzCD123-12, hzCD123-13, hzCD123-14, hzCD123-15, hzCD123-16, hzCD123-17, hzCD123-18, hzCD123-19, hzCD123-20, hzCD123-21, hzCD123-22, hzCD123-23, hzCD123-24, hzCD123-25, hzCD123-26, hzCD123-27, hzCD123-28, hzCD123-29, hzCD123-30, hzCD123-31, or hzCD123-32, or a sequence substantially identical (e.g., with at least 95% identity, e.g., 95-99% identity, or up to 20, 15, 10, 8, 6, 5, 4, 3, 2, or 1 amino acid changes) to any of the aforesaid sequences.

In certain embodiments, the CD123 CAR molecule, or the CD123 antigen binding domain, includes the heavy chain variable region and/or the light chain variable region of CD123-1, CD123-2, CD123-3, CD123-4, hzCD123-1, hzCD123-2, hzCD123-3, hzCD123-4, hzCD123-5, hzCD123-6, hzCD123-7, hzCD123-8, hzCD123-9, hzCD123-10, hzCD123-11, hzCD123-12, hzCD123-13, hzCD123-14, hzCD123-15, hzCD123-16, hzCD123-17, hzCD123-18, hzCD123-19, hzCD123-20, hzCD123-21, hzCD123-22, hzCD123-23, hzCD123-24, hzCD123-25, hzCD123-26, hzCD123-27, hzCD123-28, hzCD123-29, hzCD123-30, hzCD123-31, or hzCD123-32, provided in Table 11A or 12A, or a sequence substantially identical (e.g., with at least 95% identity, e.g., 95-99% identity, or up to 20, 15, 10, 8, 6, 5, 4, 3, 2, or 1 amino acid changes) to any of the aforesaid sequences.

In certain embodiments, the CD123 CAR molecule, or the CD123 antigen binding domain, includes one, two or three CDRs from the heavy chain variable region (e.g., HCDR1, HCDR2 and/or HCDR3), provided in Table 1A or 3A; and/or one, two or three CDRs from the light chain variable region (e.g., LCDR1, LCDR2 and/or LCDR3) of CD123-1, CD123-2, CD123-3, CD123-4, hzCD123-1, hzCD123-2, hzCD123-3, hzCD123-4, hzCD123-5, hzCD123-6, hzCD123-7, hzCD123-8, hzCD123-9, hzCD123-10, hzCD123-11, hzCD123-12, hzCD123-13, hzCD123-14, hzCD123-15, hzCD123-16, hzCD123-17, hzCD123-18, hzCD123-19, hzCD123-20, hzCD123-21, hzCD123-22, hzCD123-23, hzCD123-24, hzCD123-25, hzCD123-26, hzCD123-27, hzCD123-28, hzCD123-29, hzCD123-30, hzCD123-31, or hzCD123-32, provided in Table 2A or 4A; or a sequence substantially identical (e.g., at least 95% identical, e.g., 95-99% identical, or up to 5, 4, 3, 2, or 1 amino acid changes) to any of the aforesaid sequences.

In certain embodiments, the CD123 CAR molecule, or the CD123 antigen binding domain, includes one, two or three CDRs from the heavy chain variable region (e.g., HCDR1, HCDR2 and/or HCDR3), provided in Table 5A; and/or one, two or three CDRs from the light chain variable region (e.g., LCDR1, LCDR2 and/or LCDR3) of CD123-1, CD123-2, CD123-3, CD123-4, hzCD123-1, hzCD123-2, hzCD123-3, hzCD123-4, hzCD123-5, hzCD123-6, hzCD123-7, hzCD123-8, hzCD123-9, hzCD123-10, hzCD123-11, hzCD123-12, hzCD123-13, hzCD123-14, hzCD123-15, hzCD123-16, hzCD123-17, hzCD123-18, hzCD123-19, hzCD123-20, hzCD123-21, hzCD123-22, hzCD123-23, hzCD123-24, hzCD123-25, hzCD123-26, hzCD123-27, hzCD123-28, hzCD123-29, hzCD123-30, hzCD123-31, or hzCD123-32, provided in Table 6A; or a sequence substantially identical (e.g., at least 95% identical, e.g., 95-99% identical, or up to 5, 4, 3, 2, or 1 amino acid changes) to any of the aforesaid sequences.

In certain embodiments, the CD123 molecule, or the CD123 antigen binding domain, includes one, two or three CDRs from the heavy chain variable region (e.g., HCDR1, HCDR2 and/or HCDR3), provided in Table 7A; and/or one, two or three CDRs from the light chain variable region (e.g., LCDR1, LCDR2 and/or LCDR3) of CD123-1, CD123-2, CD123-3, CD123-4, hzCD123-1, hzCD123-2, hzCD123-3, hzCD123-4, hzCD123-5, hzCD123-6, hzCD123-7, hzCD123-8, hzCD123-9, hzCD123-10, hzCD123-11, hzCD123-12, hzCD123-13, hzCD123-14, hzCD123-15, hzCD123-16, hzCD123-17, hzCD123-18, hzCD123-19, hzCD123-20, hzCD123-21, hzCD123-22, hzCD123-23, hzCD123-24, hzCD123-25, hzCD123-26, hzCD123-27, hzCD123-28, hzCD123-29, hzCD123-30, hzCD123-31, or hzCD123-32, provided in Table 8A; or a sequence substantially identical (e.g., at least 95% identical, e.g., 95-99% identical, or up to 5, 4, 3, 2, or 1 amino acid changes) to any of the aforesaid sequences.

The sequences of CDR sequences of the scFv domains are shown in Tables, 3A, 5A, and 7A for the heavy chain variable domains and in Tables 2A, 4A, 6A, and 8A for the light chain variable domains. “ID” stands for the respective SEQ ID NO for each CDR.

The CDRs provided in Tables 1A, 2A, 3A, and 4A are according to a combination of the Kabat and Chothia numbering scheme.

TABLE 1A
Heavy Chain Variable Domain CDRs

| Candidate | HCDR1 | ID | HCDR2 | ID | HCDR3 | ID |
|---|---|---|---|---|---|---|
| CAR123-2 | GYTFTGYYMH | 335 | WINPNSGGTNYAQKFQG | 363 | DMNILATVPFDI | 391 |
| CAR123-3 | GYIFTGYYIH | 337 | WINPNSGGTNYAQKFQG | 364 | DMNILATVPFDI | 392 |
| CAR123-4 | GYTFTGYYMH | 336 | WINPNSGGTNYAQKFQG | 365 | DMNILATVPFDI | 393 |
| CAR123-1 | GYTFTDYYMH | 334 | WINPNSGDTNYAQKFQG | 362 | DMNILATVPFDI | 390 |

TABLE 2A
Light Chain Variable Domain CDRs

| Candidate | LCDR1 | ID | LCDR2 | ID | LCDR3 | ID |
|---|---|---|---|---|---|---|
| CAR123-2 | RASQSISSYLN | 419 | AAFSLQS | 447 | QQGDSVPLT | 475 |
| CAR123-3 | RASQSISSYLN | 420 | AASSLQS | 448 | QQGDSVPLT | 476 |
| CAR123-4 | RASQSISSYLN | 421 | AASSLQS | 449 | QQGDSVPLT | 477 |
| CAR123-1 | RASQSISTYLN | 418 | AASSLQS | 446 | QQGDSVPLT | 474 |

TABLE 3A
Heavy Chain Variable Domain CDR

| | HCDR1 | ID | HCDR2 | ID | HCDR3 | ID |
|---|---|---|---|---|---|---|
| hzCAR123 | GYTFTSYWMN | 361 | RIDPYDSETHYNQK | 389 | GNWDDY | 417 |
| | | | FKD | | | |

TABLE 4A
Light Chain Variable Domain CDR

| | LCDR1 | ID | LCDR2 | ID | LCDR3 | ID |
|---|---|---|---|---|---|---|
| hzCAR123 | RASKSISKDLA | 445 | SGSTLQS | 473 | QQHNKYPYT | 47 |

TABLE 5A
Heavy Chain Variable Domain CDRs according to the Kabat
numbering scheme (Kabat et al. (1991), “Sequences of
Proteins of Immunological Interest,” 5th Ed. Public
Health Service, National Institutes of Health, Bethesda, MD)

| Candidate | HCDR1 | ID | HCDR2 | ID | HCDR3 | ID |
|---|---|---|---|---|---|---|
| CAR123-2 | GYYMH | 487 | WINPNSGGTNYAQKFQG | 492 | DMNILATVPFDI | 497 |
| CAR123-3 | GYYIH | 488 | WINPNSGGTNYAQKFQG | 493 | DMNILATVPFDI | 498 |
| CAR123-4 | DYYMH | 489 | WINPNSGDTNYAQKFQG | 494 | DMNILATVPFDI | 499 |
| CAR123-1 | GYYMH | 486 | WINPNSGGTNYAQKFQG | 491 | DMNILATVPFDI | 496 |
| hzCAR123-1 | SYWMN | 490 | RIDPYDSETHYNQKFKD | 495 | GNWDDY | 500 |
| hzCAR123-2 | SYWMN | 490 | RIDPYDSETHYNQKFKD | 495 | GNWDDY | 500 |
| hzCAR123-3 | SYWMN | 490 | RIDPYDSETHYNQKFKD | 495 | GNWDDY | 500 |
| hzCAR123-4 | SYWMN | 490 | RIDPYDSETHYNQKFKD | 495 | GNWDDY | 500 |
| hzCAR123-5 | SYWMN | 490 | RIDPYDSETHYNQKFKD | 495 | GNWDDY | 500 |
| hzCAR123-6 | SYWMN | 490 | RIDPYDSETHYNQKFKD | 495 | GNWDDY | 500 |
| hzCAR123-7 | SYWMN | 490 | RIDPYDSETHYNQKFKD | 495 | GNWDDY | 500 |
| hzCAR123-8 | SYWMN | 490 | RIDPYDSETHYNQKFKD | 495 | GNWDDY | 500 |
| hzCAR123-9 | SYWMN | 490 | RIDPYDSETHYNQKFKD | 495 | GNWDDY | 500 |
| hzCAR123-10 | SYWMN | 490 | RIDPYDSETHYNQKFKD | 495 | GNWDDY | 500 |
| hzCAR123-11 | SYWMN | 490 | RIDPYDSETHYNQKFKD | 495 | GNWDDY | 500 |
| hzCAR123-12 | SYWMN | 490 | RIDPYDSETHYNQKFKD | 495 | GNWDDY | 500 |
| hzCAR123-13 | SYWMN | 490 | RIDPYDSETHYNQKFKD | 495 | GNWDDY | 500 |
| hzCAR123-14 | SYWMN | 490 | RIDPYDSETHYNQKFKD | 495 | GNWDDY | 500 |
| hzCAR123-15 | SYWMN | 490 | RIDPYDSETHYNQKFKD | 495 | GNWDDY | 500 |
| hzCAR123-16 | SYWMN | 490 | RIDPYDSETHYNQKFKD | 495 | GNWDDY | 500 |
| hzCAR123-17 | SYWMN | 490 | RIDPYDSETHYNQKFKD | 495 | GNWDDY | 500 |
| hzCAR123-18 | SYWMN | 490 | RIDPYDSETHYNQKFKD | 495 | GNWDDY | 500 |
| hzCAR123-19 | SYWMN | 490 | RIDPYDSETHYNQKFKD | 495 | GNWDDY | 500 |
| hzCAR123-20 | SYWMN | 490 | RIDPYDSETHYNQKFKD | 495 | GNWDDY | 500 |
| hzCAR123-21 | SYWMN | 490 | RIDPYDSETHYNQKFKD | 495 | GNWDDY | 500 |
| hzCAR123-22 | SYWMN | 490 | RIDPYDSETHYNQKFKD | 495 | GNWDDY | 500 |
| hzCAR123-23 | SYWMN | 490 | RIDPYDSETHYNQKFKD | 495 | GNWDDY | 500 |
| hzCAR123-24 | SYWMN | 490 | RIDPYDSETHYNQKFKD | 495 | GNWDDY | 500 |
| hzCAR123-25 | SYWMN | 490 | RIDPYDSETHYNQKFKD | 495 | GNWDDY | 500 |
| hzCAR123-26 | SYWMN | 490 | RIDPYDSETHYNQKFKD | 495 | GNWDDY | 500 |
| hzCAR123-27 | SYWMN | 490 | RIDPYDSETHYNQKFKD | 495 | GNWDDY | 500 |
| hzCAR123-28 | SYWMN | 490 | RIDPYDSETHYNQKFKD | 495 | GNWDDY | 500 |
| hzCAR123-29 | SYWMN | 490 | RIDPYDSETHYNQKFKD | 495 | GNWDDY | 500 |
| hzCAR123-30 | SYWMN | 490 | RIDPYDSETHYNQKFKD | 495 | GNWDDY | 500 |
| hzCAR123-31 | SYWMN | 490 | RIDPYDSETHYNQKFKD | 495 | GNWDDY | 500 |
| hzCAR123-32 | SYWMN | 490 | RIDPYDSETHYNQKFKD | 495 | GNWDDY | 500 |

TABLE 6A
Light Chain Variable Domain CDRs according to
the Kabat numbering scheme (Kabat et al.
(1991), “Sequences of Proteins of
Immunological Interest,” 5th Ed.
Public Health Service, National Institutes of
Health, Bethesda, MD)

| Candidate | LCDR1 | ID | LCDR2 | ID | LCDR3 | ID |
|---|---|---|---|---|---|---|
| CAR123-2 | RASQSISSYLN | 502 | AASSLQS | 507 | QQGDSVPLT | 512 |
| CAR123-3 | RASQSISSYLN | 503 | AASSLQS | 508 | QQGDSVPLT | 513 |
| CAR123-4 | RASQSISSYLN | 504 | AASSLQS | 509 | QQGDSVPLT | 514 |
| CAR123-1 | RASQSISTYLN | 501 | AAFSLQS | 506 | QQGDSVPLT | 511 |
| hzCAR123-1 | RASKSISKDLA | 505 | SGSTLQS | 510 | QQHNKYPYT | 515 |
| hzCAR123-2 | RASKSISKDLA | 505 | SGSTLQS | 510 | QQHNKYPYT | 515 |
| hzCAR123-3 | RASKSISKDLA | 505 | SGSTLQS | 510 | QQHNKYPYT | 515 |
| hzCAR123-4 | RASKSISKDLA | 505 | SGSTLQS | 510 | QQHNKYPYT | 515 |
| hzCAR123-5 | RASKSISKDLA | 505 | SGSTLQS | 510 | QQHNKYPYT | 515 |
| hzCAR123-6 | RASKSISKDLA | 505 | SGSTLQS | 510 | QQHNKYPYT | 515 |
| hzCAR123-7 | RASKSISKDLA | 505 | SGSTLQS | 510 | QQHNKYPYT | 515 |
| hzCAR123-8 | RASKSISKDLA | 505 | SGSTLQS | 510 | QQHNKYPYT | 515 |
| hzCAR123-10 | RASKSISKDLA | 505 | SGSTLQS | 510 | QQHNKYPYT | 515 |
| hzCAR123-10 | RASKSISKDLA | 505 | SGSTLQS | 510 | QQHNKYPYT | 515 |
| hzCAR123-11 | RASKSISKDLA | 505 | SGSTLQS | 510 | QQHNKYPYT | 515 |
| hzCAR123-12 | RASKSISKDLA | 505 | SGSTLQS | 510 | QQHNKYPYT | 515 |
| hzCAR123-13 | RASKSISKDLA | 505 | SGSTLQS | 510 | QQHNKYPYT | 515 |
| hzCAR123-14 | RASKSISKDLA | 505 | SGSTLQS | 510 | QQHNKYPYT | 515 |
| hzCAR123-15 | RASKSISKDLA | 505 | SGSTLQS | 510 | QQHNKYPYT | 515 |
| hzCAR123-16 | RASKSISKDLA | 505 | SGSTLQS | 510 | QQHNKYPYT | 515 |
| hzCAR123-17 | RASKSISKDLA | 505 | SGSTLQS | 510 | QQHNKYPYT | 515 |
| hzCAR123-18 | RASKSISKDLA | 505 | SGSTLQS | 510 | QQHNKYPYT | 515 |
| hzCAR123-19 | RASKSISKDLA | 505 | SGSTLQS | 510 | QQHNKYPYT | 515 |
| hzCAR123-20 | RASKSISKDLA | 505 | SGSTLQS | 510 | QQHNKYPYT | 515 |
| hzCAR123-21 | RASKSISKDLA | 505 | SGSTLQS | 510 | QQHNKYPYT | 515 |
| hzCAR123-22 | RASKSISKDLA | 505 | SGSTLQS | 510 | QQHNKYPYT | 515 |
| hzCAR123-23 | RASKSISKDLA | 505 | SGSTLQS | 510 | QQHNKYPYT | 515 |
| hzCAR123-24 | RASKSISKDLA | 505 | SGSTLQS | 510 | QQHNKYPYT | 515 |
| hzCAR123-25 | RASKSISKDLA | 505 | SGSTLQS | 510 | QQHNKYPYT | 515 |
| hzCAR123-26 | RASKSISKDLA | 505 | SGSTLQS | 510 | QQHNKYPYT | 515 |
| hzCAR123-27 | RASKSISKDLA | 505 | SGSTLQS | 510 | QQHNKYPYT | 515 |
| hzCAR123-28 | RASKSISKDLA | 505 | SGSTLQS | 510 | QQHNKYPYT | 515 |
| hzCAR123-29 | RASKSISKDLA | 505 | SGSTLQS | 510 | QQHNKYPYT | 515 |
| hzCAR123-30 | RASKSISKDLA | 505 | SGSTLQS | 510 | QQHNKYPYT | 515 |
| hzCAR123-31 | RASKSISKDLA | 505 | SGSTLQS | 510 | QQHNKYPYT | 515 |
| hzCAR123-32 | RASKSISKDLA | 505 | SGSTLQS | 510 | QQHNKYPYT | 515 |

TABLE 7A
Heavy Chain Variable Domain CDRs
according to the Chothia numbering scheme
(Al-Lazikani et al., (1997) JMB 273, 927-948)

| Candidate | HCDR1 | ID | HCDR2 | ID | HCDR3 | ID |
|---|---|---|---|---|---|---|
| CAR123-2 | GYTFTGY | 517 | NPNSGG | 522 | DMNILATVPFDI | 527 |
| CAR123-3 | GYIFTGY | 518 | NPNSGG | 523 | DMNILATVPFDI | 528 |
| CAR123-4 | GYTFTDY | 519 | NPNSGD | 524 | DMNILATVPFDI | 529 |
| CAR123-1 | GYTFTGY | 516 | NPNSGG | 521 | DMNILATVPFDI | 526 |
| hzCAR123-1 | GYTFTSY | 520 | DPYDSE | 525 | GNWDDY | 530 |
| hzCAR123-2 | GYTFTSY | 520 | DPYDSE | 525 | GNWDDY | 530 |
| hzCAR123-3 | GYTFTSY | 520 | DPYDSE | 525 | GNWDDY | 530 |
| hzCAR123-4 | GYTFTSY | 520 | DPYDSE | 525 | GNWDDY | 530 |
| hzCAR123-5 | GYTFTSY | 520 | DPYDSE | 525 | GNWDDY | 530 |
| hzCAR123-6 | GYTFTSY | 520 | DPYDSE | 525 | GNWDDY | 530 |
| hzCAR123-7 | GYTFTSY | 520 | DPYDSE | 525 | GNWDDY | 530 |
| hzCAR123-8 | GYTFTSY | 520 | DPYDSE | 525 | GNWDDY | 530 |
| hzCAR123-9 | GYTFTSY | 520 | DPYDSE | 525 | GNWDDY | 530 |
| hzCAR123-10 | GYTFTSY | 520 | DPYDSE | 525 | GNWDDY | 530 |
| hzCAR123-11 | GYTFTSY | 520 | DPYDSE | 525 | GNWDDY | 530 |
| hzCAR123-12 | GYTFTSY | 520 | DPYDSE | 525 | GNWDDY | 530 |
| hzCAR123-13 | GYTFTSY | 520 | DPYDSE | 525 | GNWDDY | 530 |
| hzCAR123-14 | GYTFTSY | 520 | DPYDSE | 525 | GNWDDY | 530 |
| hzCAR123-15 | GYTFTSY | 520 | DPYDSE | 525 | GNWDDY | 530 |
| hzCAR123-16 | GYTFTSY | 520 | DPYDSE | 525 | GNWDDY | 530 |
| hzCAR123-17 | GYTFTSY | 520 | DPYDSE | 525 | GNWDDY | 530 |
| hzCAR123-18 | GYTFTSY | 520 | DPYDSE | 525 | GNWDDY | 530 |
| hzCAR123-19 | GYTFTSY | 520 | DPYDSE | 525 | GNWDDY | 530 |
| hzCAR123-20 | GYTFTSY | 520 | DPYDSE | 525 | GNWDDY | 530 |
| hzCAR123-21 | GYTFTSY | 520 | DPYDSE | 525 | GNWDDY | 530 |
| hzCAR123-22 | GYTFTSY | 520 | DPYDSE | 525 | GNWDDY | 530 |
| hzCAR123-23 | GYTFTSY | 520 | DPYDSE | 525 | GNWDDY | 530 |
| hzCAR123-24 | GYTFTSY | 520 | DPYDSE | 525 | GNWDDY | 530 |
| hzCAR123-25 | GYTFTSY | 520 | DPYDSE | 525 | GNWDDY | 530 |
| hzCAR123-26 | GYTFTSY | 520 | DPYDSE | 525 | GNWDDY | 530 |
| hzCAR123-27 | GYTFTSY | 520 | DPYDSE | 525 | GNWDDY | 530 |
| hzCAR123-28 | GYTFTSY | 520 | DPYDSE | 525 | GNWDDY | 530 |
| hzCAR123-29 | GYTFTSY | 520 | DPYDSE | 525 | GNWDDY | 530 |
| hzCAR123-30 | GYTFTSY | 520 | DPYDSE | 525 | GNWDDY | 530 |
| hzCAR123-31 | GYTFTSY | 520 | DPYDSE | 525 | GNWDDY | 530 |
| hzCAR123-32 | GYTFTSY | 520 | DPYDSE | 525 | GNWDDY | 530 |

TABLE 8A
Light Chain Variable Domain CDRs according
to the Chothia numbering scheme (Al-Lazikani
et al., (1997) JMB 273, 927-948)

| Candidate | LCDR1 | ID | LCDR2 | ID | LCDR3 | ID |
|---|---|---|---|---|---|---|
| CAR123-2 | SQSISSY | 532 | AAS | 537 | GDSVPL | 542 |
| CAR123-3 | SQSISSY | 533 | AAS | 538 | GDSVPL | 543 |
| CAR123-4 | SQSISSY | 534 | AAS | 539 | GDSVPL | 544 |
| CAR123-1 | SQSISTY | 531 | AAF | 536 | GDSVPL | 541 |
| hzCAR123-1 | SKSISKD | 535 | SGS | 540 | HNKYPY | 555 |
| hzCAR123-2 | SKSISKD | 535 | SGS | 540 | HNKYPY | 555 |
| hzCAR123-3 | SKSISKD | 535 | SGS | 540 | HNKYPY | 555 |
| hzCAR123-4 | SKSISKD | 535 | SGS | 540 | HNKYPY | 555 |
| hzCAR123-5 | SKSISKD | 535 | SGS | 540 | HNKYPY | 555 |
| hzCAR123-6 | SKSISKD | 535 | SGS | 540 | HNKYPY | 555 |
| hzCAR123-7 | SKSISKD | 535 | SGS | 540 | HNKYPY | 555 |
| hzCAR123-8 | SKSISKD | 535 | SGS | 540 | HNKYPY | 555 |
| hzCAR123-10 | SKSISKD | 535 | SGS | 540 | HNKYPY | 555 |
| hzCAR123-10 | SKSISKD | 535 | SGS | 540 | HNKYPY | 555 |
| hzCAR123-11 | SKSISKD | 535 | SGS | 540 | HNKYPY | 555 |
| hzCAR123-12 | SKSISKD | 535 | SGS | 540 | HNKYPY | 555 |
| hzCAR123-13 | SKSISKD | 535 | SGS | 540 | HNKYPY | 555 |
| hzCAR123-14 | SKSISKD | 535 | SGS | 540 | HNKYPY | 555 |
| hzCAR123-15 | SKSISKD | 535 | SGS | 540 | HNKYPY | 555 |
| hzCAR123-16 | SKSISKD | 535 | SGS | 540 | HNKYPY | 555 |
| hzCAR123-17 | SKSISKD | 535 | SGS | 540 | HNKYPY | 555 |
| hzCAR123-18 | SKSISKD | 535 | SGS | 540 | HNKYPY | 555 |
| hzCAR123-19 | SKSISKD | 535 | SGS | 540 | HNKYPY | 555 |
| hzCAR123-20 | SKSISKD | 535 | SGS | 540 | HNKYPY | 555 |
| hzCAR123-21 | SKSISKD | 535 | SGS | 540 | HNKYPY | 555 |
| hzCAR123-22 | SKSISKD | 535 | SGS | 540 | HNKYPY | 555 |
| hzCAR123-23 | SKSISKD | 535 | SGS | 540 | HNKYPY | 555 |
| hzCAR123-24 | SKSISKD | 535 | SGS | 540 | HNKYPY | 555 |
| hzCAR123-25 | SKSISKD | 535 | SGS | 540 | HNKYPY | 555 |
| hzCAR123-26 | SKSISKD | 535 | SGS | 540 | HNKYPY | 555 |
| hzCAR123-27 | SKSISKD | 535 | SGS | 540 | HNKYPY | 555 |
| hzCAR123-28 | SKSISKD | 535 | SGS | 540 | HNKYPY | 555 |
| hzCAR123-29 | SKSISKD | 535 | SGS | 540 | HNKYPY | 555 |
| hzCAR123-30 | SKSISKD | 535 | SGS | 540 | HNKYPY | 555 |
| hzCAR123-31 | SKSISKD | 535 | SGS | 540 | HNKYPY | 555 |
| hzCAR123-32 | SKSISKD | 535 | SGS | 540 | HNKYPY | 555 |

In embodiments, CD123 single chain variable fragments are generated and cloned into lentiviral CAR expression vectors with the intracellular CD3zeta domain and the intracellular co-stimulatory domain of 4-1BB. Names of exemplary fully human CD123 scFvs are depicted in Table 9A. Names of exemplary humanized CD123 scFvs are depicted in Table 10A.

TABLE 9A
CAR-CD123 constructs

| Construct ID | CAR Nickname |
|---|---|
| EBB-C1357-F11 | CAR123-1 |
| EBB-C1358-B10 | CAR123-2 |
| EBB-C1358-D5 | CAR123-3 |
| EBB-C1357-C4 | CAR123-4 |

TABLE 10A
CAR-CD123 constructs

| Construct ID | CAR Nickname |
|---|---|
| VH1_1-46_X_VK1_L8 | hzCAR-1 |
| VH1_1-46_X_VK3_L6 | hzCAR-2 |
| VH1_1-46_X_VK6_A14 | hzCAR-3 |
| VH1_1-46_X_VK4_B3 | hzCAR-4 |
| VK1_L8_X_VH1_1-46 | hzCAR-5 |
| VK3_L6_X_VH1_1-46 | hzCAR-6 |
| VK6_A14_X_VH1_1-46 | hzCAR-7 |
| VK4_B3_X_VH1_1-46 | hzCAR-8 |
| VH7_7-4.1_X_VK1_L8 | hzCAR-9 |
| VH7_7-4.1_X_VK3_L6 | hzCAR-10 |
| VH7_7-4.1_X_VK6_A14 | hzCAR-11 |
| VH7_7-4.1_X_VK4_B3 | hzCAR-12 |
| VK1_L8_X_VH7_7-4.1 | hzCAR-13 |
| VK3_L6_X_VH7_7-4.1 | hzCAR-14 |
| VK6_A14_X_VH7_7-4.1 | hzCAR-15 |
| VK4_B3_X_VH7_7-4.1 | hzCAR-16 |
| VH5_5-A_X_VK1_L8 | hzCAR-17 |
| VH5_5-A_X_VK3_L6 | hzCAR-18 |
| VH5_5-A_X_VK6_A14 | hzCAR-19 |
| VH5_5-A_X_VK4_B3 | hzCAR-20 |
| VK1_L8_X_VH5_5-A | hzCAR-21 |
| VK3_L6_X_VH5_5-A | hzCAR-22 |
| VK6_A14_X_VH5_5-A | hzCAR-23 |
| VK4_B3_X_VH5_5-A | hzCAR-24 |
| VH3_3-74_X_VK1_L8 | hzCAR-25 |
| VH3_3-74_X_VK3_L6 | hzCAR-26 |
| VH3_3-74_X_VK6_A14 | hzCAR-27 |
| VH3_3-74_X_VK4_B3 | hzCAR-28 |
| VK1_L8_X_VH3_3-74 | hzCAR-29 |
| VK3_L6_X_VH3_3-74 | hzCAR-30 |
| VK6_A14_X_VH3_3-74 | hzCAR-31 |
| VK4_B3_X_VH3_3-74 | hzCAR-32 |

In embodiments, the order in which the VL and VH domains appear in the scFv is varied (i.e., VL-VH, or VH-VL orientation), and where either three or four copies of the “G4S” (SEQ ID NO:25) subunit, in which each subunit comprises the sequence GGGGS (SEQ ID NO:25) (e.g., (G4S)₃ (SEQ ID NO:28) or (G4S)₄(SEQ ID NO:27)), connect the variable domains to create the entirety of the scFv domain, as shown in Table 11A, Table 12A, and Table 12B.

The amino acid and nucleic acid sequences of the CD123 scFv domains and CD123 CAR molecules are provided in Table 11A, Table 12A, and Table 12B. The amino acid sequences for the variable heavy chain and variable light chain for each scFv is also provided in Table 11A and Table 12A. It is noted that the scFv fragments (SEQ ID NOs: 157-160, and 184-215) with a leader sequence (e.g., the amino acid sequence of SEQ ID NO: 1 or the nucleotide sequence of SEQ ID NO: 12) and without a leader sequence (SEQ ID NOs: 478, 480, 483, 485, and 556-587) are also encompassed by the present invention.

In embodiments, these clones in Table 11A and 12A all contained a Q/K residue change in the signal domain of the co-stimulatory domain derived from CD3zeta chain.

TABLE 11A
Exemplary CD123 CAR sequences

| | SEQ | |
|---|---|---|
| Name | ID | Sequence |
| CAR123-2 | 40 | atggccctccctgtcaccgccctgctgcttccgctggctcttctgctccacgccgctcggccccaag |
| NT | | tgcaactcgtccaaagcggagcggaagtcaagaaacccggagcgagcgtgaaagtgtcctgcaa |
| | | agcctccggctacacctttacgggctactacatgcactgggtgcgccaggcaccaggacagggtc |
| | | ttgaatggatgggatggatcaaccctaattcgggcggaactaactacgcacagaagttccagggga |
| | | gagtgactctgactcgggatacctccatctcaactgtctacatggaactctcccgcttgcggtcagat |
| | | gatacggcagtgtactactgcgcccgcgacatgaatatcctggctaccgtgccgttcgacatctggg |
| | | gacaggggactatggttactgtctcatcgggcggtggaggttcaggaggaggcggctcgggagg |
| | | cggaggttcggacattcagatgacccagtccccatcctctctgtcggccagcgtcggagatagggt |
| | | gaccattacctgtcgggcctcgcaaagcatctcctcgtacctcaactggtatcagcaaaagccggg |
| | | aaaggcgcctaagctgctgatctacgccgcttcgagcttgcaaagcggggtgccatccagattctc |
| | | gggatcaggctcaggaaccgacttcaccctgaccgtgaacagcctccagccggaggactttgcca |
| | | cttactactgccagcagggagactccgtgccgcttactttcggggggggtacccgcctggagatca |
| | | agaccactaccccagcaccgaggccacccaccccggctcctaccatcgcctcccagcctctgtcc |
| | | ctgcgtccggaggcatgtagacccgcagctggtggggccgtgcatacccggggtcttgacttcgc |
| | | ctgcgatatctacatttgggcccctctggctggtacttgcggggtcctgctgctttcactcgtgatcact |
| | | ctttactgtaagcgcggtcggaagaagctgctgtacatctttaagcaacccttcatgaggcctgtgca |
| | | gactactcaagaggaggacggctgttcatgccggttcccagaggaggaggaaggcggctgcgaa |
| | | ctgcgcgtgaaattcagccgcagcgcagatgctccagcctacaagcaggggcagaaccagctct |
| | | acaacgaactcaatcttggtcggagagaggagtacgacgtgctggacaagcggagaggacggg |
| | | acccagaaatgggcgggaagccgcgcagaaagaatccccaagagggcctgtacaacgagctcc |
| | | aaaaggataagatggcagaagcctatagcgagattggtatgaaaggggaacgcagaagaggcaa |
| | | aggccacgacggactgtaccagggactcagcaccgccaccaaggacacctatgacgctcttcac |
| | | atgcaggccctgccgcctcgg |
| CAR123-2 | 99 | MALPVTALLLPLALLLHAARPQVQLVQSGAEVKKPGASVKVS |
| AA | | CKASGYTFTGYYMHWVRQAPGQGLEWMGWINPNSGGTNYA |
| | | QKFQGRVTLTRDTSISTVYMELSRLRSDDTAVYYCARDMNILA |
| | | TVPFDIWGQGTMVTVSSGGGGSGGGGSGGGGSDIQMTQSPSS |
| | | LSASVGDRVTITCRASQSISSYLNWYQQKPGKAPKLLIYAASSL |
| | | QSGVPSRFSGSGSGTDFTLTVNSLQPEDFATYYCQQGDSVPLTF |
| | | GGGTRLEIKTTTPAPRPPTPAPTIASQPLSLRPEACRPAAGGAV |
| | | HTRGLDFACDIYIWAPLAGTCGVLLLSLVITLYCKRGRKKLLYI |
| | | FKQPFMRPVQTTQEEDGCSCRFPEEEEGGCELRVKFSRSADAP |
| | | AYKQGQNQLYNELNLGRREEYDVLDKRRGRDPEMGGKPRRK |
| | | NPQEGLYNELQKDKMAEAYSEIGMKGERRRGKGHDGLYQGL |
| | | STATKDTYDALHMQALPPR |
| CAR123-2 | 158 | MALPVTALLLPLALLLHAARPQVQLVQSGAEVKKPGASVKVS |
| scFv | | CKASGYTFTGYYMHWVRQAPGQGLEWMGWINPNSGGTNYA |
| | | QKFQGRVTLTRDTSISTVYMELSRLRSDDTAVYYCARDMNILA |
| | | TVPFDIWGQGTMVTVSSGGGGSGGGGSGGGGSDIQMTQSPSS |
| | | LSASVGDRVTITCRASQSISSYLNWYQQKPGKAPKLLIYAASSL |
| | | QSGVPSRFSGSGSGTDFTLTVNSLQPEDFATYYCQQGDSVPLTF |
| | | GGGTRLEIK |
| CAR123-2 | 217 | QVQLVQSGAEVKKPGASVKVSCKASGYTFTGYYMHWVRQAP |
| VH | | GQGLEWMGWINPNSGGTNYAQKFQGRVTLTRDTSISTVYMEL |
| | | SRLRSDDTAVYYCARDMNILATVPFDIWGQGTMVTVSS |
| CAR123-2 | 276 | DIQMTQSPSSLSASVGDRVTITCRASQSISSYLNWYQQKPGKAP |
| VL | | KLLIYAASSLQSGVPSRFSGSGSGTDFTLTVNSLQPEDFATYYC |
| | | QQGDSVPLTFGGGTRLEIK |
| CAR123-3 | 41 | atggccctccctgtcaccgccctgctgcttccgctggctcttctgctccacgccgctcggccccaag |
| NT | | tccaactcgttcaatccggcgcagaagtcaagaagccaggagcatcagtgaaagtgtcctgcaaa |
| | | gcctcaggctacatcttcacgggatactacatccactgggtgcgccaggctccgggccagggcctt |
| | | gagtggatgggctggatcaaccctaactctgggggaaccaactacgctcagaagttccaggggag |
| | | ggtcactatgactcgcgatacctccatctccactgcgtacatggaactctcgggactgagatccgac |
| | | gatcctgccgtgtactactgcgcccgggacatgaacatcttggcgaccgtgccgtttgacatttggg |
| | | gacagggcaccctcgtcactgtgtcgagcggtggaggaggctcggggggtggcggatcaggag |
| | | ggggaggaagcgacatccagctgactcagagcccatcgtcgttgtccgcgtcggtgggggatag |
| | | agtgaccattacttgccgcgccagccagagcatctcatcatatctgaattggtaccagcagaagccc |
| | | ggaaaggccccaaaactgctgatctacgctgcaagcagcctccaatcgggagtgccgtcacggtt |
| | | ctccgggtccggttcgggaactgactttaccctgaccgtgaattcgctgcaaccggaggatttcgcc |
| | | acgtactactgtcagcaaggagactccgtgccgctgaccttcggtggaggcaccaaggtcgaaat |
| | | caagaccactaccccagcaccgaggccacccaccccggctcctaccatcgcctcccagcctctgt |
| | | ccctgcgtccggaggcatgtagacccgcagctggtggggccgtgcatacccggggtcttgacttc |
| | | gcctgcgatatctacatttgggcccctctggctggtacttgcggggtcctgctgctttcactcgtgatc |
| | | actctttactgtaagcgcggtcggaagaagctgctgtacatctttaagcaacccttcatgaggcctgt |
| | | gcagactactcaagaggaggacggctgttcatgccggttcccagaggaggaggaaggcggctgc |
| | | gaactgcgcgtgaaattcagccgcagcgcagatgctccagcctacaagcaggggcagaaccagc |
| | | tctacaacgaactcaatcttggtcggagagaggagtacgacgtgctggacaagcggagaggacg |
| | | ggacccagaaatgggcgggaagccgcgcagaaagaatccccaagagggcctgtacaacgagct |
| | | ccaaaaggataagatggcagaagcctatagcgagattggtatgaaaggggaacgcagaagaggc |
| | | aaaggccacgacggactgtaccagggactcagcaccgccaccaaggacacctatgacgctcttc |
| | | acatgcaggccctgccgcctcgg |
| CAR123-3 | 100 | MALPVTALLLPLALLLHAARPQVQLVQSGAEVKKPGASVKVS |
| AA | | CKASGYIFTGYYIHWVRQAPGQGLEWMGWINPNSGGTNYAQ |
| | | KFQGRVTMTRDTSISTAYMELSGLRSDDPAVYYCARDMNILA |
| | | TVPFDIWGQGTLVTVSSGGGGSGGGGSGGGGSDIQLTQSPSSL |
| | | SASVGDRVTITCRASQSISSYLNWYQQKPGKAPKLLIYAASSLQ |
| | | SGVPSRFSGSGSGTDFTLTVNSLQPEDFATYYCQQGDSVPLTFG |
| | | GGTKVEIKTTTPAPRPPTPAPTIASQPLSLRPEACRPAAGGAVH |
| | | TRGLDFACDIYIWAPLAGTCGVLLLSLVITLYCKRGRKKLLYIF |
| | | KQPFMRPVQTTQEEDGCSCRFPEEEEGGCELRVKFSRSADAPA |
| | | YKQGQNQLYNELNLGRREEYDVLDKRRGRDPEMGGKPRRKN |
| | | PQEGLYNELQKDKMAEAYSEIGMKGERRRGKGHDGLYQGLS |
| | | TATKDTYDALHMQALPPR |
| CAR123-3 | 159 | MALPVTALLLPLALLLHAARPQVQLVQSGAEVKKPGASVKVS |
| scFv | | CKASGYIFTGYYIHWVRQAPGQGLEWMGWINPNSGGTNYAQ |
| | | KFQGRVTMTRDTSISTAYMELSGLRSDDPAVYYCARDMNILA |
| | | TVPFDIWGQGTLVTVSSGGGGSGGGGSGGGGSDIQLTQSPSSL |
| | | SASVGDRVTITCRASQSISSYLNWYQQKPGKAPKLLIYAASSLQ |
| | | SGVPSRFSGSGSGTDFTLTVNSLQPEDFATYYCQQGDSVPLTFG |
| | | GGTKVEIK |
| CAR123-3 | 218 | QVQLVQSGAEVKKPGASVKVSCKASGYIFTGYYIHWVRQAPG |
| VH | | QGLEWMGWINPNSGGTNYAQKFQGRVTMTRDTSISTAYMEL |
| | | SGLRSDDPAVYYCARDMNILATVPFDIWGQGTLVTVSS |
| CAR123-3 | 277 | DIQLTQSPSSLSASVGDRVTITCRASQSISSYLNWYQQKPGKAP |
| VL | | KLLIYAASSLQSGVPSRFSGSGSGTDFTLTVNSLQPEDFATYYC |
| | | QQGDSVPLTFGGGTKVEIK |
| CAR123-4 | 42 | atggccctccctgtcaccgccctgctgcttccgctggctcttctgctccacgccgctcggccccaag |
| NT | | tccaactccaacagtcaggcgcagaagtgaaaaagagcggtgcatcggtgaaagtgtcatgcaaa |
| | | gcctcgggctacaccttcactgactactatatgcactggctgcggcaggcaccgggacagggactt |
| | | gagtggatgggatggatcaacccgaattcaggggacactaactacgcgcagaagttccagggga |
| | | gagtgaccctgacgagggacacctcaatttcgaccgtctacatggaattgtcgcgcctgagatcgg |
| | | acgatactgctgtgtactactgtgcccgcgacatgaacatcctcgcgactgtgccttttgatatctggg |
| | | gacaggggactatggtcaccgtttcctccgcttccggtggcggaggctcgggaggccgggcctcc |
| | | ggtggaggaggcagcgacatccagatgactcagagcccttcctcgctgagcgcctcagtgggag |
| | | atcgcgtgaccatcacttgccgggccagccagtccatttcgtcctacctcaattggtaccagcagaa |
| | | gccgggaaaggcgcccaagctcttgatctacgctgcgagctccctgcaaagcggggtgccgagc |
| | | cgattctcgggttccggctcgggaaccgacttcactctgaccatctcatccctgcaaccagaggact |
| | | ttgccacctactactgccaacaaggagattctgtcccactgacgttcggcggaggaaccaaggtcg |
| | | aaatcaagaccactaccccagcaccgaggccacccaccccggctcctaccatcgcctcccagcct |
| | | ctgtccctgcgtccggaggcatgtagacccgcagctggtggggccgtgcatacccggggtcttga |
| | | cttcgcctgcgatatctacatttgggcccctctggctggtacttgcggggtcctgctgctttcactcgtg |
| | | atcactctttactgtaagcgcggtcggaagaagctgctgtacatctttaagcaacccttcatgaggcct |
| | | gtgcagactactcaagaggaggacggctgttcatgccggttcccagaggaggaggaaggcggct |
| | | gcgaactgcgcgtgaaattcagccgcagcgcagatgctccagcctacaagcaggggcagaacca |
| | | gctctacaacgaactcaatcttggtcggagagaggagtacgacgtgctggacaagcggagagga |
| | | cgggacccagaaatgggcgggaagccgcgcagaaagaatccccaagagggcctgtacaacga |
| | | gctccaaaaggataagatggcagaagcctatagcgagattggtatgaaaggggaacgcagaaga |
| | | ggcaaaggccacgacggactgtaccagggactcagcaccgccaccaaggacacctatgacgct |
| | | cttcacatgcaggccctgccgcctcgg |
| CAR123-4 | 101 | MALPVTALLLPLALLLHAARPQVQLQQSGAEVKKSGASVKVS |
| AA | | CKASGYTFTDYYMHWLRQAPGQGLEWMGWINPNSGDTNYA |
| | | QKFQGRVTLTRDTSISTVYMELSRLRSDDTAVYYCARDMNILA |
| | | TVPFDIWGQGTMVTVSSASGGGGSGGRASGGGGSDIQMTQSP |
| | | SSLSASVGDRVTITCRASQSISSYLNWYQQKPGKAPKLLIYAAS |
| | | SLQSGVPSRFSGSGSGTDFTLTISSLQPEDFATYYCQQGDSVPL |
| | | TFGGGTKVEIKTTTPAPRPPTPAPTIASQPLSLRPEACRPAAGGA |
| | | VHTRGLDFACDIYIWAPLAGTCGVLLLSLVITLYCK |
| CAR123-4 | 160 | MALPVTALLLPLALLLHAARPQVQLQQSGAEVKKSGASVKVS |
| scFv | | CKASGYTFTDYYMHWLRQAPGQGLEWMGWINPNSGDTNYA |
| | | QKFQGRVTLTRDTSISTVYMELSRLRSDDTAVYYCARDMNILA |
| | | TVPFDIWGQGTMVTVSSASGGGGSGGRASGGGGSDIQMTQSP |
| | | SSLSASVGDRVTITCRASQSISSYLNWYQQKPGKAPKLLIYAAS |
| | | SLQSGVPSRFSGSGSGTDFTLTISSLQPEDFATYYCQQGDSVPL |
| | | TFGGGTKVEIK |
| CAR123-4 | 219 | QVQLQQSGAEVKKSGASVKVSCKASGYTFTDYYMHWLRQAP |
| VH | | GQGLEWMGWINPNSGDTNYAQKFQGRVTLTRDTSISTVYMEL |
| | | SRLRSDDTAVYYCARDMNILATVPFDIWGQGTMVTVSS |
| CAR123-4 | 278 | DIQMTQSPSSLSASVGDRVTITCRASQSISSYLNWYQQKPGKAP |
| VL | | KLLIYAASSLQSGVPSRFSGSGSGTDFTLTISSLQPEDFATYYCQ |
| | | QGDSVPLTFGGGTKVEIK |
| CAR123-1 | 39 | atggccctccctgtcaccgccctgctgcttccgctggctcttctgctccacgccgctcggccccaag |
| NT | | tccaactcgtccagtcaggagcggaagtcaagaagcccggagcgtcagtcaaagtgtcatgcaaa |
| | | gcctcgggctacactttcactgggtactacatgcactgggtgcgccaggctccaggacagggactg |
| | | gaatggatgggatggatcaacccgaactccggtggcaccaattacgcccagaagttccagggga |
| | | gggtgaccatgactcgcgacacgtcgatcagcaccgcatacatggagctgtcaagactccggtcc |
| | | gacgatactgccgtgtactactgcgcacgggacatgaacattctggccaccgtgccttttgacatctg |
| | | gggtcagggaactatggttaccgtgtcctctggtggaggcggctccggcggggggggaagcgga |
| | | ggcggtggaagcgacattcagatgacccagtcgccttcatccctttcggcgagcgtgggagatcg |
| | | cgtcactatcacttgtcgggcctcgcagtccatctccacctacctcaattggtaccagcagaagcca |
| | | ggaaaagcaccgaatctgctgatctacgccgcgttttccttgcaatcgggagtgccaagcagattca |
| | | gcggatcgggatcaggcactgatttcaccctcaccatcaactcgctgcaaccggaggatttcgctac |
| | | gtactattgccaacaaggagacagcgtgccgctcaccttcggcggagggactaagctggaaatca |
| | | agaccactaccccagcaccgaggccacccaccccggctcctaccatcgcctcccagcctctgtcc |
| | | ctgcgtccggaggcatgtagacccgcagctggtggggccgtgcatacccggggtcttgacttcgc |
| | | ctgcgatatctacatttgggcccctctggctggtacttgcggggtcctgctgctttcactcgtgatcact |
| | | ctttactgtaagcgcggtcggaagaagctgctgtacatctttaagcaacccttcatgaggcctgtgca |
| | | gactactcaagaggaggacggctgttcatgccggttcccagaggaggaggaaggcggctgcgaa |
| | | ctgcgcgtgaaattcagccgcagcgcagatgctccagcctacaagcaggggcagaaccagctct |
| | | acaacgaactcaatcttggtcggagagaggagtacgacgtgctggacaagcggagaggacggg |
| | | acccagaaatgggcgggaagccgcgcagaaagaatccccaagagggcctgtacaacgagctcc |
| | | aaaaggataagatggcagaagcctatagcgagattggtatgaaaggggaacgcagaagaggcaa |
| | | aggccacgacggactgtaccagggactcagcaccgccaccaaggacacctatgacgctcttcac |
| | | atgcaggccctgccgcctcgg |
| CAR123-1 | 98 | malpvtalllplalllhaarpqvqlvqsgaevkkpgasvkvsckasgytftgyymhwvrqapg |
| AA | | qglewmgwinpnsggtnyaqkfqgrvtmtrdtsistaymelsrlrsddtavyycardmnilat |
| | | vpfdiwgqgtmvtvssggggsggggsggggsdiqmtqspsslsasvgdrvtitcrasqsistyl |
| | | nwyqqkpgkapnlliyaafslqsgvpsrfsgsgsgtdftltinslqpedfatyycqqgdsvpltfg |
| | | ggtkleiktttpaprpptpaptiasqplslrpeacrpaaggavhtrgldfacdiyiwaplagtcgvll |
| | | lslvitlyckrgrkkllyifkqpfmrpvqttqeedgcscrfpeeeeggcelrvkfsrsadapaykq |
| | | gqnqlynelnlgrreeydvldkrrgrdpemggkprrknpqeglynelqkdkmaeayseigm |
| | | kgerrrgkghdglyqglstatkdtydalhmqalppr |
| CAR123-1 | 157 | malpvtalllplalllhaarpqvqlvqsgaevkkpgasvkvsckasgytftgyymhwvrqapg |
| scFv | | qglewmgwinpnsggtnyaqkfqgrvtmtrdtsistaymelsrlrsddtavyycardmnilat |
| | | vpfdiwgqgtmvtvssggggsggggsggggsdiqmtqspsslsasvgdrvtitcrasqsistyl |
| | | nwyqqkpgkapnlliyaafslqsgvpsrfsgsgsgtdftltinslqpedfatyycqqgdsvpltfg |
| | | ggtkleik |
| CAR123-1 | 216 | QVQLVQSGAEVKKPGASVKVSCKASGYTFTGYYMHWVRQAP |
| VH | | GQGLEWMGWINPNSGGTNYAQKFQGRVTMTRDTSISTAYME |
| | | LSRLRSDDTAVYYCARDMNILATVPFDIWGQGTMVTVSS |
| CAR123-1 | 275 | DIQMTQSPSSLSASVGDRVTITCRASQSISTYLNWYQQKPGKAP |
| VL | | NLLIYAAFSLQSGVPSRFSGSGSGTDFTLTINSLQPEDFATYYCQ |
| | | QGDSVPLTFGGGTKLEIK |

TABLE 12A
Humanized CD123 CAR Sequences

| Name | SEQ ID | Sequence |
|---|---|---|
| hzCAR123- | 66 | ATGGCCCTCCCTGTCACCGCCCTGCTGCTTCCGCTGGCTCTTCTG |
| 1 NT | | CTCCACGCCGCTCGGCCCCAAGTGCAGCTGGTCCAGTCGGGAGC |
| | | CGAAGTCAAGAAGCCCGGCGCTAGCGTGAAAGTGTCCTGCAAAG |
| | | CCTCCGGGTACACATTCACCTCCTACTGGATGAATTGGGTCAGAC |
| | | AGGCGCCCGGCCAGGGACTCGAGTGGATGGGAAGGATTGATCCT |
| | | TACGACTCCGAAACCCATTACAACCAGAAGTTCAAGGACCGCGT |
| | | GACCATGACTGTGGATAAGTCCACTTCCACCGCTTACATGGAGCT |
| | | GTCCAGCCTGCGCTCCGAGGATACCGCAGTGTACTACTGCGCCC |
| | | GGGGAAACTGGGACGACTATTGGGGACAGGGAACTACCGTGAC |
| | | CGTGTCAAGCGGGGGTGGCGGTAGCGGAGGAGGGGGCTCCGGC |
| | | GGCGGCGGCTCAGGGGGCGGAGGAAGCGACGTGCAGCTCACCC |
| | | AGTCGCCCTCATTTCTGTCGGCCTCAGTGGGAGACAGAGTGACC |
| | | ATTACTTGTCGGGCCTCCAAGAGCATCTCCAAGGACCTGGCCTG |
| | | GTATCAGCAGAAGCCAGGAAAGGCGCCTAAGTTGCTCATCTACT |
| | | CGGGGTCGACCCTGCAATCTGGCGTGCCGTCCCGGTTCTCCGGTT |
| | | CGGGAAGCGGTACCGAATTCACCCTTACTATCTCCTCCCTGCAAC |
| | | CGGAGGACTTCGCCACCTACTACTGCCAACAGCACAACAAGTAC |
| | | CCGTACACTTTCGGGGGTGGCACGAAGGTCGAAATCAAGACCAC |
| | | TACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 125 | MALPVTALLLPLALLLHAARPQVQLVQSGAEVKKPGASVKVSCKA |
| 1 AA | | SGYTFTSYWMNWVRQAPGQGLEWMGRIDPYDSETHYNQKFKDRV |
| | | TMTVDKSTSTAYMELSSLRSEDTAVYYCARGNWDDYWGQGTTVT |
| | | VSSGGGGSGGGGSGGGGSGGGGSDVQLTQSPSFLSASVGDRVTITC |
| | | RASKSISKDLAWYQQKPGKAPKLLIYSGSTLQSGVPSRFSGSGSGTE |
| | | FTLTISSLQPEDFATYYCQQHNKYPYTFGGGTKVEIKTTTPAPRPPTP |
| | | APTIASQPLSLRPEACRPAAGGAVHTRGLDFACDIYIWAPLAGTCGV |
| | | LLLSLVITLYCKRGRKKLLYIFKQPFMRPVQTTQEEDGCSCRFPEEEE |
| | | GGCELRVKFSRSADAPAYKQGQNQLYNELNLGRREEYDVLDKRRG |
| | | RDPEMGGKPRRKNPQEGLYNELQKDKMAEAYSEIGMKGERRRGK |
| | | GHDGLYQGLSTATKDTYDALHMQALPPR |
| hzCAR123-1 | 184 | MALPVTALLLPLALLLHAARPQVQLVQSGAEVKKPGASVKVSCKA |
| scFv | | SGYTFTSYWMNWVRQAPGQGLEWMGRIDPYDSETHYNQKFKDRV |
| | | TMTVDKSTSTAYMELSSLRSEDTAVYYCARGNWDDYWGQGTTVT |
| | | VSSGGGGSGGGGSGGGGSGGGGSDVQLTQSPSFLSASVGDRVTITC |
| | | RASKSISKDLAWYQQKPGKAPKLLIYSGSTLQSGVPSRFSGSGSGTE |
| | | FTLTISSLQPEDFATYYCQQHNKYPYTFGGGTKVEIK |
| hzCAR123- | 243 | QVQLVQSGAEVKKPGASVKVSCKASGYTFTSYWMNWVRQAPGQG |
| 1 VH | | LEWMGRIDPYDSETHYNQKFKDRVTMTVDKSTSTAYMELSSLRSE |
| | | DTAVYYCARGNWDDYWGQGTTVTVSS |
| hzCAR123- | 302 | DVQLTQSPSFLSASVGDRVTITCRASKSISKDLAWYQQKPGKAPKLL |
| 1 VL | | IYSGSTLQSGVPSRFSGSGSGTEFTLTISSLQPEDFATYYCQQHNKYP |
| | | YTFGGGTKVEIK |
| hzCAR123- | 67 | ATGGCCCTCCCTGTCACCGCCCTGCTGCTTCCGCTGGCTCTTCTG |
| 2 NT | | CTCCACGCCGCTCGGCCCCAAGTGCAGCTGGTCCAGTCGGGAGC |
| | | CGAAGTCAAGAAGCCCGGCGCTAGCGTGAAAGTGTCCTGCAAAG |
| | | CCTCCGGGTACACATTCACCTCCTACTGGATGAATTGGGTCAGAC |
| | | AGGCGCCCGGCCAGGGACTCGAGTGGATGGGAAGGATTGATCCT |
| | | TACGACTCCGAAACCCATTACAACCAGAAGTTCAAGGACCGCGT |
| | | GACCATGACTGTGGATAAGTCCACTTCCACCGCTTACATGGAGCT |
| | | GTCCAGCCTGCGCTCCGAGGATACCGCAGTGTACTACTGCGCCC |
| | | GGGGAAACTGGGACGACTATTGGGGACAGGGAACTACCGTGAC |
| | | CGTGTCAAGCGGGGGTGGCGGTAGCGGAGGAGGGGGCTCCGGC |
| | | GGCGGCGGCTCAGGGGGCGGAGGAAGCGAAGTGGTGCTGACCC |
| | | AGTCGCCCGCAACCCTCTCTCTGTCGCCGGGAGAACGCGCCACT |
| | | CTTTCCTGTCGGGCGTCCAAGAGCATCTCAAAGGACCTCGCCTGG |
| | | TACCAGCAGAAGCCTGGTCAAGCCCCGCGGCTGCTGATCTACTC |
| | | CGGCTCCACGCTGCAATCAGGAATCCCAGCCAGATTTTCCGGTTC |
| | | GGGGTCGGGGACTGACTTCACCTTGACCATTAGCTCGCTGGAAC |
| | | CTGAGGACTTCGCCGTGTATTACTGCCAGCAGCACAACAAGTAC |
| | | CCGTACACCTTCGGAGGCGGTACTAAGGTCGAGATCAAGACCAC |
| | | TACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 126 | MALPVTALLLPLALLLHAARPQVQLVQSGAEVKKPGASVKVSCKA |
| 2 AA | | SGYTFTSYWMNWVRQ |
| | | APGQGLEWMGRIDPYDSETHYNQKFKDRVTMTVDKSTSTAYMELS |
| | | SLRSEDTAVYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDFT |
| | | LTISSLEPEDFA |
| | | VYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123-2 | 185 | MALPVTALLLPLALLLHAARPQVQLVQSGAEVKKPGASVKVSCKA |
| scFv | | SGYTFTSYWMNWVRQ |
| | | APGQGLEWMGRIDPYDSETHYNQKFKDRVTMTVDKSTSTAYMELS |
| | | SLRSEDTAVYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDFT |
| | | LTISSLEPEDFA |
| | | VYYCQQHNKYPYTFGGGTKVEIK |
| hzCAR123- | 244 | QVQLVQSGAEVKKPGASVKVSCKASGYTFTSYWMNWVRQAPGQG |
| 2 VH | | LEWMGRIDPYDSETHYNQKFKDRVTMTVDKSTSTAYMELSSLRSE |
| | | DTAVYYCARGNWDDYWGQGTTVTVSS |
| hzCAR123- | 303 | EVVLTQSPATLSLSPGERATLSCRASKSISKDLAWYQQKPGQAPRLL |
| 2 VL | | IYSGSTLQSGIPARFSGSGSGTDFTLTISSLEPEDFAVYYCQQHNKYP |
| | | YTFGGGTKVEIK |
| hzCAR123- | 68 | ATGGCCCTCCCTGTCACCGCCCTGCTGCTTCCGCTGGCTCTTCTG |
| 3 NT | | CTCCACGCCGCTCGGCCCCAAGTGCAGCTGGTCCAGTCGGGAGC |
| | | CGAAGTCAAGAAGCCCGGCGCTAGCGTGAAAGTGTCCTGCAAAG |
| | | CCTCCGGGTACACATTCACCTCCTACTGGATGAATTGGGTCAGAC |
| | | AGGCGCCCGGCCAGGGACTCGAGTGGATGGGAAGGATTGATCCT |
| | | TACGACTCCGAAACCCATTACAACCAGAAGTTCAAGGACCGCGT |
| | | GACCATGACTGTGGATAAGTCCACTTCCACCGCTTACATGGAGCT |
| | | GTCCAGCCTGCGCTCCGAGGATACCGCAGTGTACTACTGCGCCC |
| | | GGGGAAACTGGGACGACTATTGGGGACAGGGAACTACCGTGAC |
| | | CGTGTCAAGCGGGGGTGGCGGTAGCGGAGGAGGGGGCTCCGGC |
| | | GGCGGCGGCTCAGGGGGCGGAGGAAGCGACGTCGTGATGACCC |
| | | AGTCACCGGCATTCCTGTCCGTGACTCCCGGAGAAAAGGTCACG |
| | | ATTACTTGCCGGGCGTCCAAGAGCATCTCCAAGGACCTCGCCTG |
| | | GTACCAACAGAAGCCGGACCAGGCCCCTAAGCTGTTGATCTACT |
| | | CGGGGTCCACCCTTCAATCGGGAGTGCCATCGCGGTTTAGCGGTT |
| | | CGGGTTCTGGGACCGACTTCACTTTCACCATCTCCTCACTGGAAG |
| | | CCGAGGATGCCGCCACTTACTACTGTCAGCAGCACAACAAGTAT |
| | | CCGTACACCTTCGGAGGCGGTACCAAAGTGGAGATCAAGACCAC |
| | | TACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 127 | MALPVTALLLPLALLLHAARPQVQLVQSGAEVKKPGASVKVSCKA |
| 3 AA | | SGYTFTSYWMNWVRQ |
| | | APGQGLEWMGRIDPYDSETHYNQKFKDRVTMTVDKSTSTAYMELS |
| | | SLRSEDTAVYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123-3 | 186 | MALPVTALLLPLALLLHAARPQVQLVQSGAEVKKPGASVKVSCKA |
| scFv | | SGYTFTSYWMNWVRQ |
| | | APGQGLEWMGRIDPYDSETHYNQKFKDRVTMTVDKSTSTAYMELS |
| | | SLRSEDTAVYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIK |
| hzCAR123- | 245 | QVQLVQSGAEVKKPGASVKVSCKASGYTFTSYWMNWVRQAPGQG |
| 3 VH | | LEWMGRIDPYDSETHYNQKFKDRVTMTVDKSTSTAYMELSSLRSE |
| | | DTAVYYCARGNWDDYWGQGTTVTVSS |
| hzCAR123- | 304 | DVVMTQSPAFLSVTPGEKVTITCRASKSISKDLAWYQQKPDQAPKL |
| 3 VL | | LIYSGSTLQSGVPSRFSGSGSGTDFTFTISSLEAEDAATYYCQQHNKY |
| | | PYTFGGGTKVEIK |
| hzCAR123- | 69 | ATGGCCCTCCCTGTCACCGCCCTGCTGCTTCCGCTGGCTCTTCTG |
| 4 NT | | CTCCACGCCGCTCGGCCCCAAGTGCAGCTGGTCCAGTCGGGAGC |
| | | CGAAGTCAAGAAGCCCGGCGCTAGCGTGAAAGTGTCCTGCAAAG |
| | | CCTCCGGGTACACATTCACCTCCTACTGGATGAATTGGGTCAGAC |
| | | AGGCGCCCGGCCAGGGACTCGAGTGGATGGGAAGGATTGATCCT |
| | | TACGACTCCGAAACCCATTACAACCAGAAGTTCAAGGACCGCGT |
| | | GACCATGACTGTGGATAAGTCCACTTCCACCGCTTACATGGAGCT |
| | | GTCCAGCCTGCGCTCCGAGGATACCGCAGTGTACTACTGCGCCC |
| | | GGGGAAACTGGGACGACTATTGGGGACAGGGAACTACCGTGAC |
| | | CGTGTCAAGCGGGGGTGGCGGTAGCGGAGGAGGGGGCTCCGGC |
| | | GGCGGCGGCTCAGGGGGCGGAGGAAGCGACGTGGTCATGACTC |
| | | AGTCCCCGGACTCACTCGCGGTGTCGCTTGGAGAGAGAGCGACC |
| | | ATCAACTGTCGGGCCTCAAAGAGCATCAGCAAGGACCTGGCCTG |
| | | GTACCAGCAGAAGCCGGGACAGCCGCCAAAGCTGCTGATCTACT |
| | | CCGGGTCCACCTTGCAATCTGGTGTCCCTGACCGGTTCTCCGGTT |
| | | CCGGGTCGGGTACCGACTTCACGCTCACTATTTCGTCGCTGCAAG |
| | | CCGAAGATGTGGCCGTGTACTATTGCCAACAGCACAACAAGTAC |
| | | CCCTACACTTTTGGCGGAGGCACCAAGGTGGAAATCAAGACCAC |
| | | TACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 128 | MALPVTALLLPLALLLHAARPQVQLVQSGAEVKKPGASVKVSCKA |
| 4 AA | | SGYTFTSYWMNWVRQ |
| | | APGQGLEWMGRIDPYDSETHYNQKFKDRVTMTVDKSTSTAYMELS |
| | | SLRSEDTAVYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123-4 | 187 | MALPVTALLLPLALLLHAARPQVQLVQSGAEVKKPGASVKVSCKA |
| scFv | | SGYTFTSYWMNWVRQ |
| | | APGQGLEWMGRIDPYDSETHYNQKFKDRVTMTVDKSTSTAYMELS |
| | | SLRSEDTAVYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIK |
| hzCAR123- | 246 | QVQLVQSGAEVKKPGASVKVSCKASGYTFTSYWMNWVRQAPGQG |
| 4 VH | | LEWMGRIDPYDSETHYNQKFKDRVTMTVDKSTSTAYMELSSLRSE |
| | | DTAVYYCARGNWDDYWGQGTTVTVSS |
| hzCAR123- | 305 | DVVMTQSPDSLAVSLGERATINCRASKSISKDLAWYQQKPGQPPKL |
| 4 VL | | LIYSGSTLQSGVPDRFSGSGSGTDFTLTISSLQAEDVAVYYCQQHNK |
| | | YPYTFGGGTKVEIK |
| hzCAR123- | 70 | ATGGCCCTCCCTGTCACCGCCCTGCTGCTTCCGCTGGCTCTTCTG |
| 5 NT | | CTCCACGCCGCTCGGCCCGACGTGCAGCTCACCCAGTCGCCCTCA |
| | | TTTCTGTCGGCCTCAGTGGGAGACAGAGTGACCATTACTTGTCGG |
| | | GCCTCCAAGAGCATCTCCAAGGACCTGGCCTGGTATCAGCAGAA |
| | | GCCAGGAAAGGCGCCTAAGTTGCTCATCTACTCGGGGTCGACCC |
| | | TGCAATCTGGCGTGCCGTCCCGGTTCTCCGGTTCGGGAAGCGGTA |
| | | CCGAATTCACCCTTACTATCTCCTCCCTGCAACCGGAGGACTTCG |
| | | CCACCTACTACTGCCAACAGCACAACAAGTACCCGTACACTTTC |
| | | GGGGGTGGCACGAAGGTCGAAATCAAGGGGGGTGGCGGTAGCG |
| | | GAGGAGGGGGCTCCGGCGGCGGCGGCTCAGGGGGCGGAGGAAG |
| | | CCAAGTGCAGCTGGTCCAGTCGGGAGCCGAAGTCAAGAAGCCCG |
| | | GCGCTAGCGTGAAAGTGTCCTGCAAAGCCTCCGGGTACACATTC |
| | | ACCTCCTACTGGATGAATTGGGTCAGACAGGCGCCCGGCCAGGG |
| | | ACTCGAGTGGATGGGAAGGATTGATCCTTACGACTCCGAAACCC |
| | | ATTACAACCAGAAGTTCAAGGACCGCGTGACCATGACTGTGGAT |
| | | AAGTCCACTTCCACCGCTTACATGGAGCTGTCCAGCCTGCGCTCC |
| | | GAGGATACCGCAGTGTACTACTGCGCCCGGGGAAACTGGGACGA |
| | | CTATTGGGGACAGGGAACTACCGTGACCGTGTCAAGCACCACTA |
| | | CCCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 129 | MALPVTALLLPLALLLHAARPDVQLTQSPSFLSASVGDRVTITCRAS |
| 5 AA | | KSISKDLAWYQQK |
| | | PGKAPKLLIYSGSTLQSGVPSRFSGSGSGTEFTLTISSLQPEDFATYYC |
| | | QQHNKYPYTFG |
| | | GGTKVEIKGGGGSGGGGSGGGGSGGGGSQVQLVQSGAEVKKPGAS |
| | | VKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123-5 | 188 | MALPVTALLLPLALLLHAARPDVQLTQSPSFLSASVGDRVTITCRAS |
| scFv | | KSISKDLAWYQQK |
| | | PGKAPKLLIYSGSTLQSGVPSRFSGSGSGTEFTLTISSLQPEDFATYYC |
| | | QQHNKYPYTFG |
| | | GGTKVEIKGGGGSGGGGSGGGGSGGGGSQVQLVQSGAEVKKPGAS |
| | | VKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSS |
| hzCAR123- | 247 | QVQLVQSGAEVKKPGASVKVSCKASGYTFTSYWMNWVRQAPGQG |
| 5 VH | | LEWMGRIDPYDSETHYNQKFKDRVTMTVDKSTSTAYMELSSLRSE |
| | | DTAVYYCARGNWDDYWGQGTTVTVSS |
| hzCAR123- | 306 | DVQLTQSPSFLSASVGDRVTITCRASKSISKDLAWYQQKPGKAPKLL |
| 5 VL | | IYSGSTLQSGVPSRFSGSGSGTEFTLTISSLQPEDFATYYCQQHNKYP |
| | | YTFGGGTKVEIK |
| hzCAR123- | 71 | ATGGCCCTCCCTGTCACCGCCCTGCTGCTTCCGCTGGCTCTTCTG |
| 6 NT | | CTCCACGCCGCTCGGCCCGAAGTGGTGCTGACCCAGTCGCCCGC |
| | | AACCCTCTCTCTGTCGCCGGGAGAACGCGCCACTCTTTCCTGTCG |
| | | GGCGTCCAAGAGCATCTCAAAGGACCTCGCCTGGTACCAGCAGA |
| | | AGCCTGGTCAAGCCCCGCGGCTGCTGATCTACTCCGGCTCCACGC |
| | | TGCAATCAGGAATCCCAGCCAGATTTTCCGGTTCGGGGTCGGGG |
| | | ACTGACTTCACCTTGACCATTAGCTCGCTGGAACCTGAGGACTTC |
| | | GCCGTGTATTACTGCCAGCAGCACAACAAGTACCCGTACACCTT |
| | | CGGAGGCGGTACTAAGGTCGAGATCAAGGGGGGTGGCGGTAGC |
| | | GGAGGAGGGGGCTCCGGCGGCGGCGGCTCAGGGGGCGGAGGAA |
| | | GCCAAGTGCAGCTGGTCCAGTCGGGAGCCGAAGTCAAGAAGCCC |
| | | GGCGCTAGCGTGAAAGTGTCCTGCAAAGCCTCCGGGTACACATT |
| | | CACCTCCTACTGGATGAATTGGGTCAGACAGGCGCCCGGCCAGG |
| | | GACTCGAGTGGATGGGAAGGATTGATCCTTACGACTCCGAAACC |
| | | CATTACAACCAGAAGTTCAAGGACCGCGTGACCATGACTGTGGA |
| | | TAAGTCCACTTCCACCGCTTACATGGAGCTGTCCAGCCTGCGCTC |
| | | CGAGGATACCGCAGTGTACTACTGCGCCCGGGGAAACTGGGACG |
| | | ACTATTGGGGACAGGGAACTACCGTGACCGTGTCAAGCACCACT |
| | | ACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 130 | MALPVTALLLPLALLLHAARPEVVLTQSPATLSLSPGERATLSCRAS |
| 6 AA | | KSISKDLAWYQQK |
| | | PGQAPRLLIYSGSTLQSGIPARFSGSGSGTDFTLTISSLEPEDFAVYYC |
| | | QQHNKYPYTFG |
| | | GGTKVEIKGGGGSGGGGSGGGGSGGGGSQVQLVQSGAEVKKPGAS |
| | | VKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123-6 | 189 | MALPVTALLLPLALLLHAARPEVVLTQSPATLSLSPGERATLSCRAS |
| scFv | | KSISKDLAWYQQK |
| | | PGQAPRLLIYSGSTLQSGIPARFSGSGSGTDFTLTISSLEPEDFAVYYC |
| | | QQHNKYPYTFG |
| | | GGTKVEIKGGGGSGGGGSGGGGSGGGGSQVQLVQSGAEVKKPGAS |
| | | VKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSS |
| hzCAR123- | 248 | QVQLVQSGAEVKKPGASVKVSCKASGYTFTSYWMNWVRQAPGQG |
| 6 VH | | LEWMGRIDPYDSETHYNQKFKDRVTMTVDKSTSTAYMELSSLRSE |
| | | DTAVYYCARGNWDDYWGQGTTVTVSS |
| hzCAR123- | 307 | EVVLTQSPATLSLSPGERATLSCRASKSISKDLAWYQQKPGQAPRLL |
| 6 VL | | IYSGSTLQSGIPARFSGSGSGTDFTLTISSLEPEDFAVYYCQQHNKYP |
| | | YTFGGGTKVEIK |
| hzCAR123- | 72 | ATGGCCCTCCCTGTCACCGCCCTGCTGCTTCCGCTGGCTCTTCTG |
| 7 NT | | CTCCACGCCGCTCGGCCCGACGTCGTGATGACCCAGTCACCGGC |
| | | ATTCCTGTCCGTGACTCCCGGAGAAAAGGTCACGATTACTTGCCG |
| | | GGCGTCCAAGAGCATCTCCAAGGACCTCGCCTGGTACCAACAGA |
| | | AGCCGGACCAGGCCCCTAAGCTGTTGATCTACTCGGGGTCCACC |
| | | CTTCAATCGGGAGTGCCATCGCGGTTTAGCGGTTCGGGTTCTGGG |
| | | ACCGACTTCACTTTCACCATCTCCTCACTGGAAGCCGAGGATGCC |
| | | GCCACTTACTACTGTCAGCAGCACAACAAGTATCCGTACACCTTC |
| | | GGAGGCGGTACCAAAGTGGAGATCAAGGGGGGTGGCGGTAGCG |
| | | GAGGAGGGGGCTCCGGCGGCGGCGGCTCAGGGGGCGGAGGAAG |
| | | CCAAGTGCAGCTGGTCCAGTCGGGAGCCGAAGTCAAGAAGCCCG |
| | | GCGCTAGCGTGAAAGTGTCCTGCAAAGCCTCCGGGTACACATTC |
| | | ACCTCCTACTGGATGAATTGGGTCAGACAGGCGCCCGGCCAGGG |
| | | ACTCGAGTGGATGGGAAGGATTGATCCTTACGACTCCGAAACCC |
| | | ATTACAACCAGAAGTTCAAGGACCGCGTGACCATGACTGTGGAT |
| | | AAGTCCACTTCCACCGCTTACATGGAGCTGTCCAGCCTGCGCTCC |
| | | GAGGATACCGCAGTGTACTACTGCGCCCGGGGAAACTGGGACGA |
| | | CTATTGGGGACAGGGAACTACCGTGACCGTGTCAAGCACCACTA |
| | | CCCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 131 | MALPVTALLLPLALLLHAARPDVVMTQSPAFLSVTPGEKVTITCRAS |
| 7 AA | | KSISKDLAWYQQK |
| | | PDQAPKLLIYSGSTLQSGVPSRFSGSGSGTDFTFTISSLEAEDAATYY |
| | | CQQHNKYPYTFG |
| | | GGTKVEIKGGGGSGGGGSGGGGSGGGGSQVQLVQSGAEVKKPGAS |
| | | VKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123-7 | 190 | MALPVTALLLPLALLLHAARPDVVMTQSPAFLSVTPGEKVTITCRAS |
| scFv | | KSISKDLAWYQQK |
| | | PDQAPKLLIYSGSTLQSGVPSRFSGSGSGTDFTFTISSLEAEDAATYY |
| | | CQQHNKYPYTFG |
| | | GGTKVEIKGGGGSGGGGSGGGGSGGGGSQVQLVQSGAEVKKPGAS |
| | | VKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSS |
| hzCAR123- | 249 | QVQLVQSGAEVKKPGASVKVSCKASGYTFTSYWMNWVRQAPGQG |
| 7 VH | | LEWMGRIDPYDSETHYNQKFKDRVTMTVDKSTSTAYMELSSLRSE |
| | | DTAVYYCARGNWDDYWGQGTTVTVSS |
| hzCAR123- | 308 | DVVMTQSPAFLSVTPGEKVTITCRASKSISKDLAWYQQKPDQAPKL |
| 7 VL | | LIYSGSTLQSGVPSRFSGSGSGTDFTFTISSLEAEDAATYYCQQHNKY |
| | | PYTFGGGTKVEIK |
| hzCAR123- | 73 | ATGGCCCTCCCTGTCACCGCCCTGCTGCTTCCGCTGGCTCTTCTG |
| 8 NT | | CTCCACGCCGCTCGGCCCGACGTGGTCATGACTCAGTCCCCGGA |
| | | CTCACTCGCGGTGTCGCTTGGAGAGAGAGCGACCATCAACTGTC |
| | | GGGCCTCAAAGAGCATCAGCAAGGACCTGGCCTGGTACCAGCAG |
| | | AAGCCGGGACAGCCGCCAAAGCTGCTGATCTACTCCGGGTCCAC |
| | | CTTGCAATCTGGTGTCCCTGACCGGTTCTCCGGTTCCGGGTCGGG |
| | | TACCGACTTCACGCTCACTATTTCGTCGCTGCAAGCCGAAGATGT |
| | | GGCCGTGTACTATTGCCAACAGCACAACAAGTACCCCTACACTTT |
| | | TGGCGGAGGCACCAAGGTGGAAATCAAGGGGGGTGGCGGTAGC |
| | | GGAGGAGGGGGCTCCGGCGGCGGCGGCTCAGGGGGCGGAGGAA |
| | | GCCAAGTGCAGCTGGTCCAGTCGGGAGCCGAAGTCAAGAAGCCC |
| | | GGCGCTAGCGTGAAAGTGTCCTGCAAAGCCTCCGGGTACACATT |
| | | CACCTCCTACTGGATGAATTGGGTCAGACAGGCGCCCGGCCAGG |
| | | GACTCGAGTGGATGGGAAGGATTGATCCTTACGACTCCGAAACC |
| | | CATTACAACCAGAAGTTCAAGGACCGCGTGACCATGACTGTGGA |
| | | TAAGTCCACTTCCACCGCTTACATGGAGCTGTCCAGCCTGCGCTC |
| | | CGAGGATACCGCAGTGTACTACTGCGCCCGGGGAAACTGGGACG |
| | | ACTATTGGGGACAGGGAACTACCGTGACCGTGTCAAGCACCACT |
| | | ACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 132 | MALPVTALLLPLALLLHAARPDVVMTQSPDSLAVSLGERATINCRA |
| 8 AA | | SKSISKDLAWYQQK |
| | | PGQPPKLLIYSGSTLQSGVPDRFSGSGSGTDFTLTISSLQAEDVAVYY |
| | | CQQHNKYPYTFG |
| | | GGTKVEIKGGGGSGGGGSGGGGSGGGGSQVQLVQSGAEVKKPGAS |
| | | VKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123-8 | 191 | MALPVTALLLPLALLLHAARPDVVMTQSPDSLAVSLGERATINCRA |
| scFv | | SKSISKDLAWYQQK |
| | | PGQPPKLLIYSGSTLQSGVPDRFSGSGSGTDFTLTISSLQAEDVAVYY |
| | | CQQHNKYPYTFG |
| | | GGTKVEIKGGGGSGGGGSGGGGSGGGGSQVQLVQSGAEVKKPGAS |
| | | VKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSS |
| hzCAR123- | 250 | QVQLVQSGAEVKKPGASVKVSCKASGYTFTSYWMNWVRQAPGQG |
| 8 VH | | LEWMGRIDPYDSETHYNQKFKDRVTMTVDKSTSTAYMELSSLRSE |
| | | DTAVYYCARGNWDDYWGQGTTVTVSS |
| hzCAR123- | 309 | DVVMTQSPDSLAVSLGERATINCRASKSISKDLAWYQQKPGQPPKL |
| 8 VL | | LIYSGSTLQSGVPDRFSGSGSGTDFTLTISSLQAEDVAVYYCQQHNK |
| | | YPYTFGGGTKVEIK |
| hzCAR123- | 74 | ATGGCCCTCCCTGTCACCGCCCTGCTGCTTCCGCTGGCTCTTCTG |
| 9 NT | | CTCCACGCCGCTCGGCCCCAAGTGCAGCTGGTGCAGTCAGGCAG |
| | | CGAACTGAAGAAGCCCGGAGCCTCCGTCAAAGTGTCCTGCAAAG |
| | | CCTCGGGATACACCTTCACCTCCTACTGGATGAACTGGGTCCGCC |
| | | AGGCACCTGGACAGGGGCTGGAGTGGATGGGAAGGATCGATCC |
| | | CTACGATTCCGAAACCCATTACAATCAGAAGTTCAAGGACCGGT |
| | | TTGTGTTCTCCGTGGACAAGTCCGTGTCCACCGCCTACCTCCAAA |
| | | TTAGCAGCCTGAAGGCGGAGGATACAGCTGTCTACTACTGCGCT |
| | | CGCGGAAACTGGGATGACTATTGGGGCCAGGGAACTACCGTGAC |
| | | TGTGTCCTCCGGGGGTGGCGGTAGCGGAGGAGGGGGCTCCGGCG |
| | | GCGGCGGCTCAGGGGGCGGAGGAAGCGACGTGCAGCTCACCCA |
| | | GTCGCCCTCATTTCTGTCGGCCTCAGTGGGAGACAGAGTGACCAT |
| | | TACTTGTCGGGCCTCCAAGAGCATCTCCAAGGACCTGGCCTGGT |
| | | ATCAGCAGAAGCCAGGAAAGGCGCCTAAGTTGCTCATCTACTCG |
| | | GGGTCGACCCTGCAATCTGGCGTGCCGTCCCGGTTCTCCGGTTCG |
| | | GGAAGCGGTACCGAATTCACCCTTACTATCTCCTCCCTGCAACCG |
| | | GAGGACTTCGCCACCTACTACTGCCAACAGCACAACAAGTACCC |
| | | GTACACTTTCGGGGGTGGCACGAAGGTCGAAATCAAGACCACTA |
| | | CCCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 133 | MALPVTALLLPLALLLHAARPQVQLVQSGSELKKPGASVKVSCKAS |
| 9 AA | | GYTFTSYWMNWVRQ |
| | | APGQGLEWMGRIDPYDSETHYNQKFKDRFVFSVDKSVSTAYLQISS |
| | | LKAEDTAVYYCARG |
| | | NWDDYWGQGTTVTVSSGGGGSGGGGSGGGGSGGGGSDVQLTQSP |
| | | SFLSASVGDRVTITCR |
| | | ASKSISKDLAWYQQKPGKAPKLLIYSGSTLQSGVPSRFSGSGSGTEF |
| | | TLTISSLQPEDFA |
| | | TYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123-9 | 192 | MALPVTALLLPLALLLHAARPQVQLVQSGSELKKPGASVKVSCKAS |
| scFv | | GYTFTSYWMNWVRQ |
| | | APGQGLEWMGRIDPYDSETHYNQKFKDRFVFSVDKSVSTAYLQISS |
| | | LKAEDTAVYYCARG |
| | | NWDDYWGQGTTVTVSSGGGGSGGGGSGGGGSGGGGSDVQLTQSP |
| | | SFLSASVGDRVTITCR |
| | | ASKSISKDLAWYQQKPGKAPKLLIYSGSTLQSGVPSRFSGSGSGTEF |
| | | TLTISSLQPEDFA |
| | | TYYCQQHNKYPYTFGGGTKVEIK |
| hzCAR123- | 251 | QVQLVQSGSELKKPGASVKVSCKASGYTFTSYWMNWVRQAPGQG |
| 9 VH | | LEWMGRIDPYDSETHYNQKFKDRFVFSVDKSVSTAYLQISSLKAED |
| | | TAVYYCARGNWDDYWGQGTTVTVSS |
| hzCAR123- | 310 | DVQLTQSPSFLSASVGDRVTITCRASKSISKDLAWYQQKPGKAPKLL |
| 10 VL | | IYSGSTLQSGVPSRFSGSGSGTEFTLTISSLQPEDFATYYCQQHNKYP |
| | | YTFGGGTKVEIK |
| hzCAR123- | 75 | ATGGCCCTCCCTGTCACCGCCCTGCTGCTTCCGCTGGCTCTTCTG |
| 10 NT | | CTCCACGCCGCTCGGCCCCAAGTGCAGCTGGTGCAGTCAGGCAG |
| | | CGAACTGAAGAAGCCCGGAGCCTCCGTCAAAGTGTCCTGCAAAG |
| | | CCTCGGGATACACCTTCACCTCCTACTGGATGAACTGGGTCCGCC |
| | | AGGCACCTGGACAGGGGCTGGAGTGGATGGGAAGGATCGATCC |
| | | CTACGATTCCGAAACCCATTACAATCAGAAGTTCAAGGACCGGT |
| | | TTGTGTTCTCCGTGGACAAGTCCGTGTCCACCGCCTACCTCCAAA |
| | | TTAGCAGCCTGAAGGCGGAGGATACAGCTGTCTACTACTGCGCT |
| | | CGCGGAAACTGGGATGACTATTGGGGCCAGGGAACTACCGTGAC |
| | | TGTGTCCTCCGGGGGTGGCGGTAGCGGAGGAGGGGGCTCCGGCG |
| | | GCGGCGGCTCAGGGGGCGGAGGAAGCGAAGTGGTGCTGACCCA |
| | | GTCGCCCGCAACCCTCTCTCTGTCGCCGGGAGAACGCGCCACTCT |
| | | TTCCTGTCGGGCGTCCAAGAGCATCTCAAAGGACCTCGCCTGGT |
| | | ACCAGCAGAAGCCTGGTCAAGCCCCGCGGCTGCTGATCTACTCC |
| | | GGCTCCACGCTGCAATCAGGAATCCCAGCCAGATTTTCCGGTTCG |
| | | GGGTCGGGGACTGACTTCACCTTGACCATTAGCTCGCTGGAACCT |
| | | GAGGACTTCGCCGTGTATTACTGCCAGCAGCACAACAAGTACCC |
| | | GTACACCTTCGGAGGCGGTACTAAGGTCGAGATCAAGACCACTA |
| | | CCCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 134 | MALPVTALLLPLALLLHAARPQVQLVQSGSELKKPGASVKVSCKAS |
| 10 AA | | GYTFTSYWMNWVRQ |
| | | APGQGLEWMGRIDPYDSETHYNQKFKDRFVFSVDKSVSTAYLQISS |
| | | LKAEDTAVYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDFT |
| | | LTISSLEPEDFA |
| | | VYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 193 | MALPVTALLLPLALLLHAARPQVQLVQSGSELKKPGASVKVSCKAS |
| 10 | | GYTFTSYWMNWVRQ |
| scFv | | APGQGLEWMGRIDPYDSETHYNQKFKDRFVFSVDKSVSTAYLQISS |
| | | LKAEDTAVYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDFT |
| | | LTISSLEPEDFA |
| | | VYYCQQHNKYPYTFGGGTKVEIK |
| hzCAR123- | 252 | QVQLVQSGSELKKPGASVKVSCKASGYTFTSYWMNWVRQAPGQG |
| 10 VH | | LEWMGRIDPYDSETHYNQKFKDRFVFSVDKSVSTAYLQISSLKAED |
| | | TAVYYCARGNWDDYWGQGTTVTVSS |
| hzCAR123- | 311 | EVVLTQSPATLSLSPGERATLSCRASKSISKDLAWYQQKPGQAPRLL |
| 10 VL | | IYSGSTLQSGIPARFSGSGSGTDFTLTISSLEPEDFAVYYCQQHNKYP |
| | | YTFGGGTKVEIK |
| hzCAR123- | 76 | ATGGCCCTCCCTGTCACCGCCCTGCTGCTTCCGCTGGCTCTTCTG |
| 11 NT | | CTCCACGCCGCTCGGCCCCAAGTGCAGCTGGTGCAGTCAGGCAG |
| | | CGAACTGAAGAAGCCCGGAGCCTCCGTCAAAGTGTCCTGCAAAG |
| | | CCTCGGGATACACCTTCACCTCCTACTGGATGAACTGGGTCCGCC |
| | | AGGCACCTGGACAGGGGCTGGAGTGGATGGGAAGGATCGATCC |
| | | CTACGATTCCGAAACCCATTACAATCAGAAGTTCAAGGACCGGT |
| | | TTGTGTTCTCCGTGGACAAGTCCGTGTCCACCGCCTACCTCCAAA |
| | | TTAGCAGCCTGAAGGCGGAGGATACAGCTGTCTACTACTGCGCT |
| | | CGCGGAAACTGGGATGACTATTGGGGCCAGGGAACTACCGTGAC |
| | | TGTGTCCTCCGGGGGTGGCGGTAGCGGAGGAGGGGGCTCCGGCG |
| | | GCGGCGGCTCAGGGGGCGGAGGAAGCGACGTCGTGATGACCCA |
| | | GTCACCGGCATTCCTGTCCGTGACTCCCGGAGAAAAGGTCACGA |
| | | TTACTTGCCGGGCGTCCAAGAGCATCTCCAAGGACCTCGCCTGGT |
| | | ACCAACAGAAGCCGGACCAGGCCCCTAAGCTGTTGATCTACTCG |
| | | GGGTCCACCCTTCAATCGGGAGTGCCATCGCGGTTTAGCGGTTCG |
| | | GGTTCTGGGACCGACTTCACTTTCACCATCTCCTCACTGGAAGCC |
| | | GAGGATGCCGCCACTTACTACTGTCAGCAGCACAACAAGTATCC |
| | | GTACACCTTCGGAGGCGGTACCAAAGTGGAGATCAAGACCACTA |
| | | CCCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 135 | MALPVTALLLPLALLLHAARPQVQLVQSGSELKKPGASVKVSCKAS |
| 11 AA | | GYTFTSYWMNWVRQ |
| | | APGQGLEWMGRIDPYDSETHYNQKFKDRFVFSVDKSVSTAYLQISS |
| | | LKAEDTAVYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 194 | MALPVTALLLPLALLLHAARPQVQLVQSGSELKKPGASVKVSCKAS |
| 11 | | GYTFTSYWMNWVRQ |
| scFv | | APGQGLEWMGRIDPYDSETHYNQKFKDRFVFSVDKSVSTAYLQISS |
| | | LKAEDTAVYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIK |
| hzCAR123- | 253 | QVQLVQSGSELKKPGASVKVSCKASGYTFTSYWMNWVRQAPGQG |
| 11 VH | | LEWMGRIDPYDSETHYNQKFKDRFVFSVDKSVSTAYLQISSLKAED |
| | | TAVYYCARGNWDDYWGQGTTVTVSS |
| hzCAR123- | 312 | DVVMTQSPAFLSVTPGEKVTITCRASKSISKDLAWYQQKPDQAPKL |
| 11 VL | | LIYSGSTLQSGVPSRFSGSGSGTDFTFTISSLEAEDAATYYCQQHNKY |
| | | PYTFGGGTKVEIK |
| hzCAR123- | 77 | ATGGCCCTCCCTGTCACCGCCCTGCTGCTTCCGCTGGCTCTTCTG |
| 12 NT | | CTCCACGCCGCTCGGCCCCAAGTGCAGCTGGTGCAGTCAGGCAG |
| | | CGAACTGAAGAAGCCCGGAGCCTCCGTCAAAGTGTCCTGCAAAG |
| | | CCTCGGGATACACCTTCACCTCCTACTGGATGAACTGGGTCCGCC |
| | | AGGCACCTGGACAGGGGCTGGAGTGGATGGGAAGGATCGATCC |
| | | CTACGATTCCGAAACCCATTACAATCAGAAGTTCAAGGACCGGT |
| | | TTGTGTTCTCCGTGGACAAGTCCGTGTCCACCGCCTACCTCCAAA |
| | | TTAGCAGCCTGAAGGCGGAGGATACAGCTGTCTACTACTGCGCT |
| | | CGCGGAAACTGGGATGACTATTGGGGCCAGGGAACTACCGTGAC |
| | | TGTGTCCTCCGGGGGTGGCGGTAGCGGAGGAGGGGGCTCCGGCG |
| | | GCGGCGGCTCAGGGGGCGGAGGAAGCGACGTGGTCATGACTCA |
| | | GTCCCCGGACTCACTCGCGGTGTCGCTTGGAGAGAGAGCGACCA |
| | | TCAACTGTCGGGCCTCAAAGAGCATCAGCAAGGACCTGGCCTGG |
| | | TACCAGCAGAAGCCGGGACAGCCGCCAAAGCTGCTGATCTACTC |
| | | CGGGTCCACCTTGCAATCTGGTGTCCCTGACCGGTTCTCCGGTTC |
| | | CGGGTCGGGTACCGACTTCACGCTCACTATTTCGTCGCTGCAAGC |
| | | CGAAGATGTGGCCGTGTACTATTGCCAACAGCACAACAAGTACC |
| | | CCTACACTTTTGGCGGAGGCACCAAGGTGGAAATCAAGACCACT |
| | | ACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 136 | MALPVTALLLPLALLLHAARPQVQLVQSGSELKKPGASVKVSCKAS |
| 12 AA | | GYTFTSYWMNWVRQ |
| | | APGQGLEWMGRIDPYDSETHYNQKFKDRFVFSVDKSVSTAYLQISS |
| | | LKAEDTAVYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 195 | MALPVTALLLPLALLLHAARPQVQLVQSGSELKKPGASVKVSCKAS |
| 12 | | GYTFTSYWMNWVRQ |
| scFv | | APGQGLEWMGRIDPYDSETHYNQKFKDRFVFSVDKSVSTAYLQISS |
| | | LKAEDTAVYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIK |
| hzCAR123- | 254 | QVQLVQSGSELKKPGASVKVSCKASGYTFTSYWMNWVRQAPGQG |
| 12 VH | | LEWMGRIDPYDSETHYNQKFKDRFVFSVDKSVSTAYLQISSLKAED |
| | | TAVYYCARGNWDDYWGQGTTVTVSS |
| hzCAR123- | 313 | DVVMTQSPDSLAVSLGERATINCRASKSISKDLAWYQQKPGQPPKL |
| 12 VL | | LIYSGSTLQSGVPDRFSGSGSGTDFTLTISSLQAEDVAVYYCQQHNK |
| | | YPYTFGGGTKVEIK |
| hzCAR123- | 78 | ATGGCCCTCCCTGTCACCGCCCTGCTGCTTCCGCTGGCTCTTCTG |
| 13 NT | | CTCCACGCCGCTCGGCCCGACGTGCAGCTCACCCAGTCGCCCTCA |
| | | TTTCTGTCGGCCTCAGTGGGAGACAGAGTGACCATTACTTGTCGG |
| | | GCCTCCAAGAGCATCTCCAAGGACCTGGCCTGGTATCAGCAGAA |
| | | GCCAGGAAAGGCGCCTAAGTTGCTCATCTACTCGGGGTCGACCC |
| | | TGCAATCTGGCGTGCCGTCCCGGTTCTCCGGTTCGGGAAGCGGTA |
| | | CCGAATTCACCCTTACTATCTCCTCCCTGCAACCGGAGGACTTCG |
| | | CCACCTACTACTGCCAACAGCACAACAAGTACCCGTACACTTTC |
| | | GGGGGTGGCACGAAGGTCGAAATCAAGGGGGGTGGCGGTAGCG |
| | | GAGGAGGGGGCTCCGGCGGCGGCGGCTCAGGGGGCGGAGGAAG |
| | | CCAAGTGCAGCTGGTGCAGTCAGGCAGCGAACTGAAGAAGCCCG |
| | | GAGCCTCCGTCAAAGTGTCCTGCAAAGCCTCGGGATACACCTTC |
| | | ACCTCCTACTGGATGAACTGGGTCCGCCAGGCACCTGGACAGGG |
| | | GCTGGAGTGGATGGGAAGGATCGATCCCTACGATTCCGAAACCC |
| | | ATTACAATCAGAAGTTCAAGGACCGGTTTGTGTTCTCCGTGGACA |
| | | AGTCCGTGTCCACCGCCTACCTCCAAATTAGCAGCCTGAAGGCG |
| | | GAGGATACAGCTGTCTACTACTGCGCTCGCGGAAACTGGGATGA |
| | | CTATTGGGGCCAGGGAACTACCGTGACTGTGTCCTCCACCACTAC |
| | | CCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 137 | MALPVTALLLPLALLLHAARPDVQLTQSPSFLSASVGDRVTITCRAS |
| 13 AA | | KSISKDLAWYQQK |
| | | PGKAPKLLIYSGSTLQSGVPSRFSGSGSGTEFTLTISSLQPEDFATYYC |
| | | QQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKSV |
| | | STAYLQISSLKAEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 196 | MALPVTALLLPLALLLHAARPDVQLTQSPSFLSASVGDRVTITCRAS |
| 13 | | KSISKDLAWYQQK |
| scFv | | PGKAPKLLIYSGSTLQSGVPSRFSGSGSGTEFTLTISSLQPEDFATYYC |
| | | QQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKSV |
| | | STAYLQISSLKAEDTA |
| | | VYYCARGNWDDYWGQGTTVTVSS |
| hzCAR123- | 255 | QVQLVQSGSELKKPGASVKVSCKASGYTFTSYWMNWVRQAPGQG |
| 13 VH | | LEWMGRIDPYDSETHYNQKFKDRFVFSVDKSVSTAYLQISSLKAED |
| | | TAVYYCARGNWDDYWGQGTTVTVSS |
| hzCAR123- | 314 | DVQLTQSPSFLSASVGDRVTITCRASKSISKDLAWYQQKPGKAPKLL |
| 13 VL | | IYSGSTLQSGVPSRFSGSGSGTEFTLTISSLQPEDFATYYCQQHNKYP |
| | | YTFGGGTKVEIK |
| hzCAR123- | 79 | ATGGCCCTCCCTGTCACCGCCCTGCTGCTTCCGCTGGCTCTTCTG |
| 14 NT | | CTCCACGCCGCTCGGCCCGAAGTGGTGCTGACCCAGTCGCCCGC |
| | | AACCCTCTCTCTGTCGCCGGGAGAACGCGCCACTCTTTCCTGTCG |
| | | GGCGTCCAAGAGCATCTCAAAGGACCTCGCCTGGTACCAGCAGA |
| | | AGCCTGGTCAAGCCCCGCGGCTGCTGATCTACTCCGGCTCCACGC |
| | | TGCAATCAGGAATCCCAGCCAGATTTTCCGGTTCGGGGTCGGGG |
| | | ACTGACTTCACCTTGACCATTAGCTCGCTGGAACCTGAGGACTTC |
| | | GCCGTGTATTACTGCCAGCAGCACAACAAGTACCCGTACACCTT |
| | | CGGAGGCGGTACTAAGGTCGAGATCAAGGGGGGTGGCGGTAGC |
| | | GGAGGAGGGGGCTCCGGCGGCGGCGGCTCAGGGGGCGGAGGAA |
| | | GCCAAGTGCAGCTGGTGCAGTCAGGCAGCGAACTGAAGAAGCCC |
| | | GGAGCCTCCGTCAAAGTGTCCTGCAAAGCCTCGGGATACACCTT |
| | | CACCTCCTACTGGATGAACTGGGTCCGCCAGGCACCTGGACAGG |
| | | GGCTGGAGTGGATGGGAAGGATCGATCCCTACGATTCCGAAACC |
| | | CATTACAATCAGAAGTTCAAGGACCGGTTTGTGTTCTCCGTGGAC |
| | | AAGTCCGTGTCCACCGCCTACCTCCAAATTAGCAGCCTGAAGGC |
| | | GGAGGATACAGCTGTCTACTACTGCGCTCGCGGAAACTGGGATG |
| | | ACTATTGGGGCCAGGGAACTACCGTGACTGTGTCCTCCACCACT |
| | | ACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 138 | MALPVTALLLPLALLLHAARPEVVLTQSPATLSLSPGERATLSCRAS |
| 14 AA | | KSISKDLAWYQQK |
| | | PGQAPRLLIYSGSTLQSGIPARFSGSGSGTDFTLTISSLEPEDFAVYYC |
| | | QQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKSV |
| | | STAYLQISSLKAEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 197 | MALPVTALLLPLALLLHAARPEVVLTQSPATLSLSPGERATLSCRAS |
| 14 | | KSISKDLAWYQQK |
| scFv | | PGQAPRLLIYSGSTLQSGIPARFSGSGSGTDFTLTISSLEPEDFAVYYC |
| | | QQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKSV |
| | | STAYLQISSLKAEDTA |
| | | VYYCARGNWDDYWGQGTTVTVSS |
| hzCAR123- | 256 | QVQLVQSGSELKKPGASVKVSCKASGYTFTSYWMNWVRQAPGQG |
| 14 VH | | LEWMGRIDPYDSETHYNQKFKDRFVFSVDKSVSTAYLQISSLKAED |
| | | TAVYYCARGNWDDYWGQGTTVTVSS |
| hzCAR123- | 315 | EVVLTQSPATLSLSPGERATLSCRASKSISKDLAWYQQKPGQAPRLL |
| 14 VL | | IYSGSTLQSGIPARFSGSGSGTDFTLTISSLEPEDFAVYYCQQHNKYP |
| | | YTFGGGTKVEIK |
| hzCAR123- | 80 | ATGGCCCTCCCTGTCACCGCCCTGCTGCTTCCGCTGGCTCTTCTG |
| 15 NT | | CTCCACGCCGCTCGGCCCGACGTCGTGATGACCCAGTCACCGGC |
| | | ATTCCTGTCCGTGACTCCCGGAGAAAAGGTCACGATTACTTGCCG |
| | | GGCGTCCAAGAGCATCTCCAAGGACCTCGCCTGGTACCAACAGA |
| | | AGCCGGACCAGGCCCCTAAGCTGTTGATCTACTCGGGGTCCACC |
| | | CTTCAATCGGGAGTGCCATCGCGGTTTAGCGGTTCGGGTTCTGGG |
| | | ACCGACTTCACTTTCACCATCTCCTCACTGGAAGCCGAGGATGCC |
| | | GCCACTTACTACTGTCAGCAGCACAACAAGTATCCGTACACCTTC |
| | | GGAGGCGGTACCAAAGTGGAGATCAAGGGGGGTGGCGGTAGCG |
| | | GAGGAGGGGGCTCCGGCGGCGGCGGCTCAGGGGGCGGAGGAAG |
| | | CCAAGTGCAGCTGGTGCAGTCAGGCAGCGAACTGAAGAAGCCCG |
| | | GAGCCTCCGTCAAAGTGTCCTGCAAAGCCTCGGGATACACCTTC |
| | | ACCTCCTACTGGATGAACTGGGTCCGCCAGGCACCTGGACAGGG |
| | | GCTGGAGTGGATGGGAAGGATCGATCCCTACGATTCCGAAACCC |
| | | ATTACAATCAGAAGTTCAAGGACCGGTTTGTGTTCTCCGTGGACA |
| | | AGTCCGTGTCCACCGCCTACCTCCAAATTAGCAGCCTGAAGGCG |
| | | GAGGATACAGCTGTCTACTACTGCGCTCGCGGAAACTGGGATGA |
| | | CTATTGGGGCCAGGGAACTACCGTGACTGTGTCCTCCACCACTAC |
| | | CCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 139 | MALPVTALLLPLALLLHAARPDVVMTQSPAFLSVTPGEKVTITCRAS |
| 15 AA | | KSISKDLAWYQQK |
| | | PDQAPKLLIYSGSTLQSGVPSRFSGSGSGTDFTFTISSLEAEDAATYY |
| | | CQQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKSV |
| | | STAYLQISSLKAEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 198 | MALPVTALLLPLALLLHAARPDVVMTQSPAFLSVTPGEKVTITCRAS |
| 15 | | KSISKDLAWYQQK |
| scFv | | PDQAPKLLIYSGSTLQSGVPSRFSGSGSGTDFTFTISSLEAEDAATYY |
| | | CQQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKSV |
| | | STAYLQISSLKAEDTA |
| | | VYYCARGNWDDYWGQGTTVTVSS |
| hzCAR123- | 257 | QVQLVQSGSELKKPGASVKVSCKASGYTFTSYWMNWVRQAPGQG |
| 15 VH | | LEWMGRIDPYDSETHYNQKFKDRFVFSVDKSVSTAYLQISSLKAED |
| | | TAVYYCARGNWDDYWGQGTTVTVSS |
| hzCAR123- | 316 | DVVMTQSPAFLSVTPGEKVTITCRASKSISKDLAWYQQKPDQAPKL |
| 15 VL | | LIYSGSTLQSGVPSRFSGSGSGTDFTFTISSLEAEDAATYYCQQHNKY |
| | | PYTFGGGTKVEIK |
| hzCAR123- | 81 | ATGGCCCTCCCTGTCACCGCCCTGCTGCTTCCGCTGGCTCTTCTG |
| 16 NT | | CTCCACGCCGCTCGGCCCGACGTGGTCATGACTCAGTCCCCGGA |
| | | CTCACTCGCGGTGTCGCTTGGAGAGAGAGCGACCATCAACTGTC |
| | | GGGCCTCAAAGAGCATCAGCAAGGACCTGGCCTGGTACCAGCAG |
| | | AAGCCGGGACAGCCGCCAAAGCTGCTGATCTACTCCGGGTCCAC |
| | | CTTGCAATCTGGTGTCCCTGACCGGTTCTCCGGTTCCGGGTCGGG |
| | | TACCGACTTCACGCTCACTATTTCGTCGCTGCAAGCCGAAGATGT |
| | | GGCCGTGTACTATTGCCAACAGCACAACAAGTACCCCTACACTTT |
| | | TGGCGGAGGCACCAAGGTGGAAATCAAGGGGGGTGGCGGTAGC |
| | | GGAGGAGGGGGCTCCGGCGGCGGCGGCTCAGGGGGCGGAGGAA |
| | | GCCAAGTGCAGCTGGTGCAGTCAGGCAGCGAACTGAAGAAGCCC |
| | | GGAGCCTCCGTCAAAGTGTCCTGCAAAGCCTCGGGATACACCTT |
| | | CACCTCCTACTGGATGAACTGGGTCCGCCAGGCACCTGGACAGG |
| | | GGCTGGAGTGGATGGGAAGGATCGATCCCTACGATTCCGAAACC |
| | | CATTACAATCAGAAGTTCAAGGACCGGTTTGTGTTCTCCGTGGAC |
| | | AAGTCCGTGTCCACCGCCTACCTCCAAATTAGCAGCCTGAAGGC |
| | | GGAGGATACAGCTGTCTACTACTGCGCTCGCGGAAACTGGGATG |
| | | ACTATTGGGGCCAGGGAACTACCGTGACTGTGTCCTCCACCACT |
| | | ACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 140 | MALPVTALLLPLALLLHAARPDVVMTQSPDSLAVSLGERATINCRA |
| 16 AA | | SKSISKDLAWYQQK |
| | | PGQPPKLLIYSGSTLQSGVPDRFSGSGSGTDFTLTISSLQAEDVAVYY |
| | | CQQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKSV |
| | | STAYLQISSLKAEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 199 | MALPVTALLLPLALLLHAARPDVVMTQSPDSLAVSLGERATINCRA |
| 16 | | SKSISKDLAWYQQK |
| scFv | | PGQPPKLLIYSGSTLQSGVPDRFSGSGSGTDFTLTISSLQAEDVAVYY |
| | | CQQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKSV |
| | | STAYLQISSLKAEDTA |
| | | VYYCARGNWDDYWGQGTTVTVSS |
| hzCAR123- | 258 | QVQLVQSGSELKKPGASVKVSCKASGYTFTSYWMNWVRQAPGQG |
| 16 VH | | LEWMGRIDPYDSETHYNQKFKDRFVFSVDKSVSTAYLQISSLKAED |
| | | TAVYYCARGNWDDYWGQGTTVTVSS |
| hzCAR123- | 317 | DVVMTQSPDSLAVSLGERATINCRASKSISKDLAWYQQKPGQPPKL |
| 16 VL | | LIYSGSTLQSGVPDRFSGSGSGTDFTLTISSLQAEDVAVYYCQQHNK |
| | | YPYTFGGGTKVEIK |
| hzCAR123- | 82 | ATGGCCCTCCCTGTCACCGCCCTGCTGCTTCCGCTGGCTCTTCTG |
| 17 NT | | CTCCACGCCGCTCGGCCCGAGGTGCAGCTGGTGCAGAGCGGAGC |
| | | CGAGGTCAAGAAGCCTGGAGAATCCCTGAGGATCAGCTGCAAAG |
| | | GCAGCGGGTATACCTTCACCTCCTACTGGATGAATTGGGTCCGCC |
| | | AGATGCCCGGAAAAGGCCTGGAGTGGATGGGACGGATTGACCCC |
| | | TACGACTCGGAAACCCATTACAACCAGAAGTTCAAGGATCACGT |
| | | GACCATCTCCGTGGACAAGTCCATTTCCACTGCGTACCTCCAGTG |
| | | GTCAAGCCTGAAGGCCTCCGACACTGCTATGTACTACTGCGCAC |
| | | GCGGAAACTGGGATGATTACTGGGGACAGGGAACAACCGTGACT |
| | | GTGTCCTCCGGGGGTGGCGGTAGCGGAGGAGGGGGCTCCGGCGG |
| | | CGGCGGCTCAGGGGGCGGAGGAAGCGACGTGCAGCTCACCCAG |
| | | TCGCCCTCATTTCTGTCGGCCTCAGTGGGAGACAGAGTGACCATT |
| | | ACTTGTCGGGCCTCCAAGAGCATCTCCAAGGACCTGGCCTGGTA |
| | | TCAGCAGAAGCCAGGAAAGGCGCCTAAGTTGCTCATCTACTCGG |
| | | GGTCGACCCTGCAATCTGGCGTGCCGTCCCGGTTCTCCGGTTCGG |
| | | GAAGCGGTACCGAATTCACCCTTACTATCTCCTCCCTGCAACCGG |
| | | AGGACTTCGCCACCTACTACTGCCAACAGCACAACAAGTACCCG |
| | | TACACTTTCGGGGGTGGCACGAAGGTCGAAATCAAGACCACTAC |
| | | CCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 141 | MALPVTALLLPLALLLHAARPEVQLVQSGAEVKKPGESLRISCKGS |
| 17 AA | | GYTFTSYWMNWVRQ |
| | | MPGKGLEWMGRIDPYDSETHYNQKFKDHVTISVDKSISTAYLQWSS |
| | | LKASDTAMYYCARG |
| | | NWDDYWGQGTTVTVSSGGGGSGGGGSGGGGSGGGGSDVQLTQSP |
| | | SFLSASVGDRVTITCR |
| | | ASKSISKDLAWYQQKPGKAPKLLIYSGSTLQSGVPSRFSGSGSGTEF |
| | | TLTISSLQPEDFA |
| | | TYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 200 | MALPVTALLLPLALLLHAARPEVQLVQSGAEVKKPGESLRISCKGS |
| 17 | | GYTFTSYWMNWVRQ |
| scFv | | MPGKGLEWMGRIDPYDSETHYNQKFKDHVTISVDKSISTAYLQWSS |
| | | LKASDTAMYYCARG |
| | | NWDDYWGQGTTVTVSSGGGGSGGGGSGGGGSGGGGSDVQLTQSP |
| | | SFLSASVGDRVTITCR |
| | | ASKSISKDLAWYQQKPGKAPKLLIYSGSTLQSGVPSRFSGSGSGTEF |
| | | TLTISSLQPEDFA |
| | | TYYCQQHNKYPYTFGGGTKVEIK |
| hzCAR123- | 259 | EVQLVQSGAEVKKPGESLRISCKGSGYTFTSYWMNWVRQMPGKGL |
| 17 VH | | EWMGRIDPYDSETHYNQKFKDHVTISVDKSISTAYLQWSSLKASDT |
| | | AMYYCARGNWDDYWGQGTTVTVSS |
| hzCAR123- | 318 | DVQLTQSPSFLSASVGDRVTITCRASKSISKDLAWYQQKPGKAPKLL |
| 17 VL | | IYSGSTLQSGVPSRFSGSGSGTEFTLTISSLQPEDFATYYCQQHNKYP |
| | | YTFGGGTKVEIK |
| hzCAR123- | 83 | ATGGCCCTCCCTGTCACCGCCCTGCTGCTTCCGCTGGCTCTTCTG |
| 18 NT | | CTCCACGCCGCTCGGCCCGAGGTGCAGCTGGTGCAGAGCGGAGC |
| | | CGAGGTCAAGAAGCCTGGAGAATCCCTGAGGATCAGCTGCAAAG |
| | | GCAGCGGGTATACCTTCACCTCCTACTGGATGAATTGGGTCCGCC |
| | | AGATGCCCGGAAAAGGCCTGGAGTGGATGGGACGGATTGACCCC |
| | | TACGACTCGGAAACCCATTACAACCAGAAGTTCAAGGATCACGT |
| | | GACCATCTCCGTGGACAAGTCCATTTCCACTGCGTACCTCCAGTG |
| | | GTCAAGCCTGAAGGCCTCCGACACTGCTATGTACTACTGCGCAC |
| | | GCGGAAACTGGGATGATTACTGGGGACAGGGAACAACCGTGACT |
| | | GTGTCCTCCGGGGGTGGCGGTAGCGGAGGAGGGGGCTCCGGCGG |
| | | CGGCGGCTCAGGGGGCGGAGGAAGCGAAGTGGTGCTGACCCAG |
| | | TCGCCCGCAACCCTCTCTCTGTCGCCGGGAGAACGCGCCACTCTT |
| | | TCCTGTCGGGCGTCCAAGAGCATCTCAAAGGACCTCGCCTGGTA |
| | | CCAGCAGAAGCCTGGTCAAGCCCCGCGGCTGCTGATCTACTCCG |
| | | GCTCCACGCTGCAATCAGGAATCCCAGCCAGATTTTCCGGTTCGG |
| | | GGTCGGGGACTGACTTCACCTTGACCATTAGCTCGCTGGAACCTG |
| | | AGGACTTCGCCGTGTATTACTGCCAGCAGCACAACAAGTACCCG |
| | | TACACCTTCGGAGGCGGTACTAAGGTCGAGATCAAGACCACTAC |
| | | CCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 142 | MALPVTALLLPLALLLHAARPEVQLVQSGAEVKKPGESLRISCKGS |
| 18 AA | | GYTFTSYWMNWVRQ |
| | | MPGKGLEWMGRIDPYDSETHYNQKFKDHVTISVDKSISTAYLQWSS |
| | | LKASDTAMYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDFT |
| | | LTISSLEPEDFA |
| | | VYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 201 | MALPVTALLLPLALLLHAARPEVQLVQSGAEVKKPGESLRISCKGS |
| 18 | | GYTFTSYWMNWVRQ |
| scFv | | MPGKGLEWMGRIDPYDSETHYNQKFKDHVTISVDKSISTAYLQWSS |
| | | LKASDTAMYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDFT |
| | | LTISSLEPEDFA |
| | | VYYCQQHNKYPYTFGGGTKVEIK |
| hzCAR123- | 260 | EVQLVQSGAEVKKPGESLRISCKGSGYTFTSYWMNWVRQMPGKGL |
| 18 VH | | EWMGRIDPYDSETHYNQKFKDHVTISVDKSISTAYLQWSSLKASDT |
| | | AMYYCARGNWDDYWGQGTTVTVSS |
| hzCAR123- | 319 | EVVLTQSPATLSLSPGERATLSCRASKSISKDLAWYQQKPGQAPRLL |
| 18 VL | | IYSGSTLQSGIPARFSGSGSGTDFTLTISSLEPEDFAVYYCQQHNKYP |
| | | YTFGGGTKVEIK |
| hzCAR123- | 84 | ATGGCCCTCCCTGTCACCGCCCTGCTGCTTCCGCTGGCTCTTCTG |
| 19 NT | | CTCCACGCCGCTCGGCCCGAGGTGCAGCTGGTGCAGAGCGGAGC |
| | | CGAGGTCAAGAAGCCTGGAGAATCCCTGAGGATCAGCTGCAAAG |
| | | GCAGCGGGTATACCTTCACCTCCTACTGGATGAATTGGGTCCGCC |
| | | AGATGCCCGGAAAAGGCCTGGAGTGGATGGGACGGATTGACCCC |
| | | TACGACTCGGAAACCCATTACAACCAGAAGTTCAAGGATCACGT |
| | | GACCATCTCCGTGGACAAGTCCATTTCCACTGCGTACCTCCAGTG |
| | | GTCAAGCCTGAAGGCCTCCGACACTGCTATGTACTACTGCGCAC |
| | | GCGGAAACTGGGATGATTACTGGGGACAGGGAACAACCGTGACT |
| | | GTGTCCTCCGGGGGTGGCGGTAGCGGAGGAGGGGGCTCCGGCGG |
| | | CGGCGGCTCAGGGGGCGGAGGAAGCGACGTCGTGATGACCCAG |
| | | TCACCGGCATTCCTGTCCGTGACTCCCGGAGAAAAGGTCACGAT |
| | | TACTTGCCGGGCGTCCAAGAGCATCTCCAAGGACCTCGCCTGGT |
| | | ACCAACAGAAGCCGGACCAGGCCCCTAAGCTGTTGATCTACTCG |
| | | GGGTCCACCCTTCAATCGGGAGTGCCATCGCGGTTTAGCGGTTCG |
| | | GGTTCTGGGACCGACTTCACTTTCACCATCTCCTCACTGGAAGCC |
| | | GAGGATGCCGCCACTTACTACTGTCAGCAGCACAACAAGTATCC |
| | | GTACACCTTCGGAGGCGGTACCAAAGTGGAGATCAAGACCACTA |
| | | CCCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 143 | MALPVTALLLPLALLLHAARPEVQLVQSGAEVKKPGESLRISCKGS |
| 19 AA | | GYTFTSYWMNWVRQ |
| | | MPGKGLEWMGRIDPYDSETHYNQKFKDHVTISVDKSISTAYLQWSS |
| | | LKASDTAMYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 202 | MALPVTALLLPLALLLHAARPEVQLVQSGAEVKKPGESLRISCKGS |
| 19 | | GYTFTSYWMNWVRQ |
| scFv | | MPGKGLEWMGRIDPYDSETHYNQKFKDHVTISVDKSISTAYLQWSS |
| | | LKASDTAMYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIK |
| hzCAR123- | 261 | EVQLVQSGAEVKKPGESLRISCKGSGYTFTSYWMNWVRQMPGKGL |
| 19 VH | | EWMGRIDPYDSETHYNQKFKDHVTISVDKSISTAYLQWSSLKASDT |
| | | AMYYCARGNWDDYWGQGTTVTVSS |
| hzCAR123- | 320 | DVVMTQSPAFLSVTPGEKVTITCRASKSISKDLAWYQQKPDQAPKL |
| 19 VL | | LIYSGSTLQSGVPSRFSGSGSGTDFTFTISSLEAEDAATYYCQQHNKY |
| | | PYTFGGGTKVEIK |
| hzCAR123- | 85 | ATGGCCCTCCCTGTCACCGCCCTGCTGCTTCCGCTGGCTCTTCTG |
| 20 NT | | CTCCACGCCGCTCGGCCCGAGGTGCAGCTGGTGCAGAGCGGAGC |
| | | CGAGGTCAAGAAGCCTGGAGAATCCCTGAGGATCAGCTGCAAAG |
| | | GCAGCGGGTATACCTTCACCTCCTACTGGATGAATTGGGTCCGCC |
| | | AGATGCCCGGAAAAGGCCTGGAGTGGATGGGACGGATTGACCCC |
| | | TACGACTCGGAAACCCATTACAACCAGAAGTTCAAGGATCACGT |
| | | GACCATCTCCGTGGACAAGTCCATTTCCACTGCGTACCTCCAGTG |
| | | GTCAAGCCTGAAGGCCTCCGACACTGCTATGTACTACTGCGCAC |
| | | GCGGAAACTGGGATGATTACTGGGGACAGGGAACAACCGTGACT |
| | | GTGTCCTCCGGGGGTGGCGGTAGCGGAGGAGGGGGCTCCGGCGG |
| | | CGGCGGCTCAGGGGGCGGAGGAAGCGACGTGGTCATGACTCAGT |
| | | CCCCGGACTCACTCGCGGTGTCGCTTGGAGAGAGAGCGACCATC |
| | | AACTGTCGGGCCTCAAAGAGCATCAGCAAGGACCTGGCCTGGTA |
| | | CCAGCAGAAGCCGGGACAGCCGCCAAAGCTGCTGATCTACTCCG |
| | | GGTCCACCTTGCAATCTGGTGTCCCTGACCGGTTCTCCGGTTCCG |
| | | GGTCGGGTACCGACTTCACGCTCACTATTTCGTCGCTGCAAGCCG |
| | | AAGATGTGGCCGTGTACTATTGCCAACAGCACAACAAGTACCCC |
| | | TACACTTTTGGCGGAGGCACCAAGGTGGAAATCAAGACCACTAC |
| | | CCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 144 | MALPVTALLLPLALLLHAARPEVQLVQSGAEVKKPGESLRISCKGS |
| 20 AA | | GYTFTSYWMNWVRQ |
| | | MPGKGLEWMGRIDPYDSETHYNQKFKDHVTISVDKSISTAYLQWSS |
| | | LKASDTAMYYCARG |
| | | NWDDYWGQGTTVTVSSGGGGSGGGGSGGGGSGGGGSDVVMTQS |
| | | PDSLAVSLGERATINCR |
| | | CATTACAACCAGAAGTTCAAGGATCACGTGACCATCTCCGTGGA |
| | | CAAGTCCATTTCCACTGCGTACCTCCAGTGGTCAAGCCTGAAGGC |
| | | CTCCGACACTGCTATGTACTACTGCGCACGCGGAAACTGGGATG |
| | | ATTACTGGGGACAGGGAACAACCGTGACTGTGTCCTCCACCACT |
| | | ACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 145 | MALPVTALLLPLALLLHAARPDVQLTQSPSFLSASVGDRVTITCRAS |
| 21 AA | | KSISKDLAWYQQK |
| | | PGKAPKLLIYSGSTLQSGVPSRFSGSGSGTEFTLTISSLQPEDFATYYC |
| | | QQHNKYPYTFG |
| | | GGTKVEIKGGGGSGGGGSGGGGSGGGGSEVQLVQSGAEVKKPGES |
| | | LRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 204 | MALPVTALLLPLALLLHAARPDVQLTQSPSFLSASVGDRVTITCRAS |
| 21 | | KSISKDLAWYQQK |
| scFv | | PGKAPKLLIYSGSTLQSGVPSRFSGSGSGTEFTLTISSLQPEDFATYYC |
| | | QQHNKYPYTFG |
| | | GGTKVEIKGGGGSGGGGSGGGGSGGGGSEVQLVQSGAEVKKPGES |
| | | LRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSS |
| hzCAR123- | 263 | EVQLVQSGAEVKKPGESLRISCKGSGYTFTSYWMNWVRQMPGKGL |
| 21 VH | | EWMGRIDPYDSETHYNQKFKDHVTISVDKSISTAYLQWSSLKASDT |
| | | AMYYCARGNWDDYWGQGTTVTVSS |
| hzCAR123- | 322 | DVQLTQSPSFLSASVGDRVTITCRASKSISKDLAWYQQKPGKAPKLL |
| 21 VL | | IYSGSTLQSGVPSRFSGSGSGTEFTLTISSLQPEDFATYYCQQHNKYP |
| | | YTFGGGTKVEIK |
| hzCAR123- | 87 | ATGGCCCTCCCTGTCACCGCCCTGCTGCTTCCGCTGGCTCTTCTG |
| 22 NT | | CTCCACGCCGCTCGGCCCGAAGTGGTGCTGACCCAGTCGCCCGC |
| | | AACCCTCTCTCTGTCGCCGGGAGAACGCGCCACTCTTTCCTGTCG |
| | | GGCGTCCAAGAGCATCTCAAAGGACCTCGCCTGGTACCAGCAGA |
| | | AGCCTGGTCAAGCCCCGCGGCTGCTGATCTACTCCGGCTCCACGC |
| | | TGCAATCAGGAATCCCAGCCAGATTTTCCGGTTCGGGGTCGGGG |
| | | ACTGACTTCACCTTGACCATTAGCTCGCTGGAACCTGAGGACTTC |
| | | GCCGTGTATTACTGCCAGCAGCACAACAAGTACCCGTACACCTT |
| | | CGGAGGCGGTACTAAGGTCGAGATCAAGGGGGGTGGCGGTAGC |
| | | GGAGGAGGGGGCTCCGGCGGCGGCGGCTCAGGGGGCGGAGGAA |
| | | GCGAGGTGCAGCTGGTGCAGAGCGGAGCCGAGGTCAAGAAGCC |
| | | TGGAGAATCCCTGAGGATCAGCTGCAAAGGCAGCGGGTATACCT |
| | | TCACCTCCTACTGGATGAATTGGGTCCGCCAGATGCCCGGAAAA |
| | | GGCCTGGAGTGGATGGGACGGATTGACCCCTACGACTCGGAAAC |
| | | CCATTACAACCAGAAGTTCAAGGATCACGTGACCATCTCCGTGG |
| | | ACAAGTCCATTTCCACTGCGTACCTCCAGTGGTCAAGCCTGAAG |
| | | GCCTCCGACACTGCTATGTACTACTGCGCACGCGGAAACTGGGA |
| | | TGATTACTGGGGACAGGGAACAACCGTGACTGTGTCCTCCACCA |
| | | CTACCCCAGCACCGAGGCCACCCACCCCGGCTCCTACCATCGCCT |
| | | CCCAGCCTCTGTCCCTGCGTCCGGAggcatgtagacccgcagctggtggggccgtg |
| | | catacccggggtcttgacttcgcctgcgatatctacatttgggcccctctggctggtacttgcggggtcctgct |
| | | gctttcactcgtgatcactctttactgtaagcgcggtcggaagaagctgctgtacatctttaagcaacccttcat |
| | | gaggcctgtgcagactactcaagaggaggacggctgttcatgccggttcccagaggaggaggaaggcgg |
| | | ctgcgaactgcgcgtgaaattcagccgcagcgcagatgctccagcctacaagcaggggcagaaccagct |
| | | ctacaacgaactcaatcttggtcggagagaggagtacgacgtgctggacaagcggagaggacgggaccc |
| | | agaaatgggcgggaagccgcgcagaaagaatccccaagagggcctgtacaacgagctccaaaaggata |
| | | agatggcagaagcctatagcgagattggtatgaaaggggaacgcagaagaggcaaaggccacgacgga |
| | | ctgtaccagggactcagcaccgccaccaaggacacctatgacgctcttcacatgcaggccctgccgcctcgg |
| hzCAR123- | 146 | MALPVTALLLPLALLLHAARPEVVLTQSPATLSLSPGERATLSCRAS |
| 22 AA | | KSISKDLAWYQQK |
| | | PGQAPRLLIYSGSTLQSGIPARFSGSGSGTDFTLTISSLEPEDFAVYYC |
| | | QQHNKYPYTFG |
| | | GGTKVEIKGGGGSGGGGSGGGGSGGGGSEVQLVQSGAEVKKPGES |
| | | LRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 205 | MALPVTALLLPLALLLHAARPEVVLTQSPATLSLSPGERATLSCRAS |
| 22 | | KSISKDLAWYQQK |
| scFv | | PGQAPRLLIYSGSTLQSGIPARFSGSGSGTDFTLTISSLEPEDFAVYYC |
| | | QQHNKYPYTFG |
| | | GGTKVEIKGGGGSGGGGSGGGGSGGGGSEVQLVQSGAEVKKPGES |
| | | LRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSS |
| hzCAR123- | 264 | EVQLVQSGAEVKKPGESLRISCKGSGYTFTSYWMNWVRQMPGKGL |
| 22 VH | | EWMGRIDPYDSETHYNQKFKDHVTISVDKSISTAYLQWSSLKASDT |
| | | AMYYCARGNWDDYWGQGTTVTVSS |
| hzCAR123- | 323 | EVVLTQSPATLSLSPGERATLSCRASKSISKDLAWYQQKPGQAPRLL |
| 22 VL | | IYSGSTLQSGIPARFSGSGSGTDFTLTISSLEPEDFAVYYCQQHNKYP |
| | | YTFGGGTKVEIK |
| hzCAR123- | 88 | ATGGCCCTCCCTGTCACCGCCCTGCTGCTTCCGCTGGCTCTTCTG |
| 23 NT | | CTCCACGCCGCTCGGCCCGACGTCGTGATGACCCAGTCACCGGC |
| | | ATTCCTGTCCGTGACTCCCGGAGAAAAGGTCACGATTACTTGCCG |
| | | GGCGTCCAAGAGCATCTCCAAGGACCTCGCCTGGTACCAACAGA |
| | | AGCCGGACCAGGCCCCTAAGCTGTTGATCTACTCGGGGTCCACC |
| | | CTTCAATCGGGAGTGCCATCGCGGTTTAGCGGTTCGGGTTCTGGG |
| | | ACCGACTTCACTTTCACCATCTCCTCACTGGAAGCCGAGGATGCC |
| | | GCCACTTACTACTGTCAGCAGCACAACAAGTATCCGTACACCTTC |
| | | GGAGGCGGTACCAAAGTGGAGATCAAGGGGGGTGGCGGTAGCG |
| | | GAGGAGGGGGCTCCGGCGGCGGCGGCTCAGGGGGCGGAGGAAG |
| | | CGAGGTGCAGCTGGTGCAGAGCGGAGCCGAGGTCAAGAAGCCT |
| | | GGAGAATCCCTGAGGATCAGCTGCAAAGGCAGCGGGTATACCTT |
| | | CACCTCCTACTGGATGAATTGGGTCCGCCAGATGCCCGGAAAAG |
| | | GCCTGGAGTGGATGGGACGGATTGACCCCTACGACTCGGAAACC |
| | | CATTACAACCAGAAGTTCAAGGATCACGTGACCATCTCCGTGGA |
| | | CAAGTCCATTTCCACTGCGTACCTCCAGTGGTCAAGCCTGAAGGC |
| | | CTCCGACACTGCTATGTACTACTGCGCACGCGGAAACTGGGATG |
| | | ATTACTGGGGACAGGGAACAACCGTGACTGTGTCCTCCACCACT |
| | | ACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 147 | MALPVTALLLPLALLLHAARPDVVMTQSPAFLSVTPGEKVTITCRAS |
| 23 AA | | KSISKDLAWYQQK |
| | | PDQAPKLLIYSGSTLQSGVPSRFSGSGSGTDFTFTISSLEAEDAATYY |
| | | CQQHNKYPYTFG |
| | | GGTKVEIKGGGGSGGGGSGGGGSGGGGSEVQLVQSGAEVKKPGES |
| | | LRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 206 | MALPVTALLLPLALLLHAARPDVVMTQSPAFLSVTPGEKVTITCRAS |
| 23 | | KSISKDLAWYQQK |
| scFv | | PDQAPKLLIYSGSTLQSGVPSRFSGSGSGTDFTFTISSLEAEDAATYY |
| | | CQQHNKYPYTFG |
| | | GGTKVEIKGGGGSGGGGSGGGGSGGGGSEVQLVQSGAEVKKPGES |
| | | LRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSS |
| hzCAR123- | 265 | EVQLVQSGAEVKKPGESLRISCKGSGYTFTSYWMNWVRQMPGKGL |
| 23 VH | | EWMGRIDPYDSETHYNQKFKDHVTISVDKSISTAYLQWSSLKASDT |
| | | AMYYCARGNWDDYWGQGTTVTVSS |
| hzCAR123- | 324 | DVVMTQSPAFLSVTPGEKVTITCRASKSISKDLAWYQQKPDQAPKL |
| 23 VL | | LIYSGSTLQSGVPSRFSGSGSGTDFTFTISSLEAEDAATYYCQQHNKY |
| | | PYTFGGGTKVEIK |
| hzCAR123- | 89 | ATGGCCCTCCCTGTCACCGCCCTGCTGCTTCCGCTGGCTCTTCTG |
| 24 NT | | CTCCACGCCGCTCGGCCCGACGTGGTCATGACTCAGTCCCCGGA |
| | | CTCACTCGCGGTGTCGCTTGGAGAGAGAGCGACCATCAACTGTC |
| | | GGGCCTCAAAGAGCATCAGCAAGGACCTGGCCTGGTACCAGCAG |
| | | AAGCCGGGACAGCCGCCAAAGCTGCTGATCTACTCCGGGTCCAC |
| | | CTTGCAATCTGGTGTCCCTGACCGGTTCTCCGGTTCCGGGTCGGG |
| | | TACCGACTTCACGCTCACTATTTCGTCGCTGCAAGCCGAAGATGT |
| | | GGCCGTGTACTATTGCCAACAGCACAACAAGTACCCCTACACTTT |
| | | TGGCGGAGGCACCAAGGTGGAAATCAAGGGGGGTGGCGGTAGC |
| | | GGAGGAGGGGGCTCCGGCGGCGGCGGCTCAGGGGGCGGAGGAA |
| | | GCGAGGTGCAGCTGGTGCAGAGCGGAGCCGAGGTCAAGAAGCC |
| | | TGGAGAATCCCTGAGGATCAGCTGCAAAGGCAGCGGGTATACCT |
| | | TCACCTCCTACTGGATGAATTGGGTCCGCCAGATGCCCGGAAAA |
| | | GGCCTGGAGTGGATGGGACGGATTGACCCCTACGACTCGGAAAC |
| | | CCATTACAACCAGAAGTTCAAGGATCACGTGACCATCTCCGTGG |
| | | ACAAGTCCATTTCCACTGCGTACCTCCAGTGGTCAAGCCTGAAG |
| | | GCCTCCGACACTGCTATGTACTACTGCGCACGCGGAAACTGGGA |
| | | TGATTACTGGGGACAGGGAACAACCGTGACTGTGTCCTCCACCA |
| | | CTACCCCAGCACCGAGGCCACCCACCCCGGCTCCTACCATCGCCT |
| | | CCCAGCCTCTGTCCCTGCGTCCGGAggcatgtagacccgcagctggtggggccgtg |
| | | catacccggggtcttgacttcgcctgcgatatctacatttgggcccctctggctggtacttgcggggtcctgct |
| | | gctttcactcgtgatcactctttactgtaagcgcggtcggaagaagctgctgtacatctttaagcaacccttcat |
| | | gaggcctgtgcagactactcaagaggaggacggctgttcatgccggttcccagaggaggaggaaggcgg |
| | | ctgcgaactgcgcgtgaaattcagccgcagcgcagatgctccagcctacaagcaggggcagaaccagct |
| | | ctacaacgaactcaatcttggtcggagagaggagtacgacgtgctggacaagcggagaggacgggaccc |
| | | agaaatgggcgggaagccgcgcagaaagaatccccaagagggcctgtacaacgagctccaaaaggata |
| | | agatggcagaagcctatagcgagattggtatgaaaggggaacgcagaagaggcaaaggccacgacgga |
| | | ctgtaccagggactcagcaccgccaccaaggacacctatgacgctcttcacatgcaggccctgccgcctcgg |
| hzCAR123- | 148 | MALPVTALLLPLALLLHAARPDVVMTQSPDSLAVSLGERATINCRA |
| 24 AA | | SKSISKDLAWYQQK |
| | | PGQPPKLLIYSGSTLQSGVPDRFSGSGSGTDFTLTISSLQAEDVAVYY |
| | | CQQHNKYPYTFG |
| | | GGTKVEIKGGGGSGGGGSGGGGSGGGGSEVQLVQSGAEVKKPGES |
| | | LRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 207 | MALPVTALLLPLALLLHAARPDVVMTQSPDSLAVSLGERATINCRA |
| 24 | | SKSISKDLAWYQQK |
| scFv | | PGQPPKLLIYSGSTLQSGVPDRFSGSGSGTDFTLTISSLQAEDVAVYY |
| | | CQQHNKYPYTFG |
| | | GGTKVEIKGGGGSGGGGSGGGGSGGGGSEVQLVQSGAEVKKPGES |
| | | LRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSS |
| hzCAR123- | 266 | EVQLVQSGAEVKKPGESLRISCKGSGYTFTSYWMNWVRQMPGKGL |
| 24 VH | | EWMGRIDPYDSETHYNQKFKDHVTISVDKSISTAYLQWSSLKASDT |
| | | AMYYCARGNWDDYWGQGTTVTVSS |
| hzCAR123- | 325 | DVVMTQSPDSLAVSLGERATINCRASKSISKDLAWYQQKPGQPPKL |
| 24 VL | | LIYSGSTLQSGVPDRFSGSGSGTDFTLTISSLQAEDVAVYYCQQHNK |
| | | YPYTFGGGTKVEIK |
| hzCAR123- | 90 | ATGGCCCTCCCTGTCACCGCCCTGCTGCTTCCGCTGGCTCTTCTG |
| 25 NT | | CTCCACGCCGCTCGGCCCGAAGTGCAGCTCGTCGAGAGCGGAGG |
| | | GGGACTGGTGCAGCCCGGAGGAAGCCTGAGGCTGTCCTGCGCTG |
| | | CCTCCGGCTACACCTTCACCTCCTACTGGATGAACTGGGTCAGAC |
| | | AGGCACCTGGAAAGGGACTGGTCTGGGTGTCGCGCATTGACCCC |
| | | TACGACTCCGAAACCCATTACAATCAGAAATTCAAGGACCGCTT |
| | | CACCATCTCCGTGGACAAAGCCAAGAGCACCGCGTACCTCCAAA |
| | | TGAACTCCCTGCGCGCTGAGGATACAGCAGTGTACTATTGCGCC |
| | | CGGGGAAACTGGGATGATTACTGGGGCCAGGGAACTACTGTGAC |
| | | TGTGTCATCCGGGGGTGGCGGTAGCGGAGGAGGGGGCTCCGGCG |
| | | GCGGCGGCTCAGGGGGCGGAGGAAGCGACGTGCAGCTCACCCA |
| | | GTCGCCCTCATTTCTGTCGGCCTCAGTGGGAGACAGAGTGACCAT |
| | | TACTTGTCGGGCCTCCAAGAGCATCTCCAAGGACCTGGCCTGGT |
| | | ATCAGCAGAAGCCAGGAAAGGCGCCTAAGTTGCTCATCTACTCG |
| | | GGGTCGACCCTGCAATCTGGCGTGCCGTCCCGGTTCTCCGGTTCG |
| | | GGAAGCGGTACCGAATTCACCCTTACTATCTCCTCCCTGCAACCG |
| | | GAGGACTTCGCCACCTACTACTGCCAACAGCACAACAAGTACCC |
| | | GTACACTTTCGGGGGTGGCACGAAGGTCGAAATCAAGACCACTA |
| | | CCCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 149 | MALPVTALLLPLALLLHAARPEVQLVESGGGLVQPGGSLRLSCAAS |
| 25 AA | | GYTFTSYWMNWVRQ |
| | | APGKGLVWVSRIDPYDSETHYNQKFKDRFTISVDKAKSTAYLQMNS |
| | | LRAEDTAVYYCARG |
| | | NWDDYWGQGTTVTVSSGGGGSGGGGSGGGGSGGGGSDVQLTQSP |
| | | SFLSASVGDRVTITCR |
| | | ASKSISKDLAWYQQKPGKAPKLLIYSGSTLQSGVPSRFSGSGSGTEF |
| | | TLTISSLQPEDFA |
| | | TYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 208 | MALPVTALLLPLALLLHAARPEVQLVESGGGLVQPGGSLRLSCAAS |
| 25 | | GYTFTSYWMNWVRQ |
| scFv | | APGKGLVWVSRIDPYDSETHYNQKFKDRFTISVDKAKSTAYLQMNS |
| | | LRAEDTAVYYCARG |
| | | NWDDYWGQGTTVTVSSGGGGSGGGGSGGGGSGGGGSDVQLTQSP |
| | | SFLSASVGDRVTITCR |
| | | ASKSISKDLAWYQQKPGKAPKLLIYSGSTLQSGVPSRFSGSGSGTEF |
| | | TLTISSLQPEDFA |
| | | TYYCQQHNKYPYTFGGGTKVEIK |
| hzCAR123- | 267 | EVQLVESGGGLVQPGGSLRLSCAASGYTFTSYWMNWVRQAPGKG |
| 25 VH | | LVWVSRIDPYDSETHYNQKFKDRFTISVDKAKSTAYLQMNSLRAED |
| | | TAVYYCARGNWDDYWGQGTTVTVSS |
| hzCAR123- | 326 | DVQLTQSPSFLSASVGDRVTITCRASKSISKDLAWYQQKPGKAPKLL |
| 25 VL | | IYSGSTLQSGVPSRFSGSGSGTEFTLTISSLQPEDFATYYCQQHNKYP |
| | | YTFGGGTKVEIK |
| hzCAR123- | 91 | ATGGCCCTCCCTGTCACCGCCCTGCTGCTTCCGCTGGCTCTTCTG |
| 26 NT | | CTCCACGCCGCTCGGCCCGAAGTGCAGCTCGTCGAGAGCGGAGG |
| | | GGGACTGGTGCAGCCCGGAGGAAGCCTGAGGCTGTCCTGCGCTG |
| | | CCTCCGGCTACACCTTCACCTCCTACTGGATGAACTGGGTCAGAC |
| | | AGGCACCTGGAAAGGGACTGGTCTGGGTGTCGCGCATTGACCCC |
| | | TACGACTCCGAAACCCATTACAATCAGAAATTCAAGGACCGCTT |
| | | CACCATCTCCGTGGACAAAGCCAAGAGCACCGCGTACCTCCAAA |
| | | TGAACTCCCTGCGCGCTGAGGATACAGCAGTGTACTATTGCGCC |
| | | CGGGGAAACTGGGATGATTACTGGGGCCAGGGAACTACTGTGAC |
| | | TGTGTCATCCGGGGGTGGCGGTAGCGGAGGAGGGGGCTCCGGCG |
| | | GCGGCGGCTCAGGGGGCGGAGGAAGCGAAGTGGTGCTGACCCA |
| | | GTCGCCCGCAACCCTCTCTCTGTCGCCGGGAGAACGCGCCACTCT |
| | | TTCCTGTCGGGCGTCCAAGAGCATCTCAAAGGACCTCGCCTGGT |
| | | ACCAGCAGAAGCCTGGTCAAGCCCCGCGGCTGCTGATCTACTCC |
| | | GGCTCCACGCTGCAATCAGGAATCCCAGCCAGATTTTCCGGTTCG |
| | | GGGTCGGGGACTGACTTCACCTTGACCATTAGCTCGCTGGAACCT |
| | | GAGGACTTCGCCGTGTATTACTGCCAGCAGCACAACAAGTACCC |
| | | GTACACCTTCGGAGGCGGTACTAAGGTCGAGATCAAGACCACTA |
| | | CCCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 150 | MALPVTALLLPLALLLHAARPEVQLVESGGGLVQPGGSLRLSCAAS |
| 26 AA | | GYTFTSYWMNWVRQ |
| | | APGKGLVWVSRIDPYDSETHYNQKFKDRFTISVDKAKSTAYLQMNS |
| | | LRAEDTAVYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDFT |
| | | LTISSLEPEDFA |
| | | VYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 209 | MALPVTALLLPLALLLHAARPEVQLVESGGGLVQPGGSLRLSCAAS |
| 26 | | GYTFTSYWMNWVRQ |
| scFv | | APGKGLVWVSRIDPYDSETHYNQKFKDRFTISVDKAKSTAYLQMNS |
| | | LRAEDTAVYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDFT |
| | | LTISSLEPEDFA |
| | | VYYCQQHNKYPYTFGGGTKVEIK |
| hzCAR123- | 268 | EVQLVESGGGLVQPGGSLRLSCAASGYTFTSYWMNWVRQAPGKG |
| 26 VH | | LVWVSRIDPYDSETHYNQKFKDRFTISVDKAKSTAYLQMNSLRAED |
| | | TAVYYCARGNWDDYWGQGTTVTVSS |
| hzCAR123- | 327 | EVVLTQSPATLSLSPGERATLSCRASKSISKDLAWYQQKPGQAPRLL |
| 26 VL | | IYSGSTLQSGIPARFSGSGSGTDFTLTISSLEPEDFAVYYCQQHNKYP |
| | | YTFGGGTKVEIK |
| hzCAR123- | 92 | ATGGCCCTCCCTGTCACCGCCCTGCTGCTTCCGCTGGCTCTTCTG |
| 27 NT | | CTCCACGCCGCTCGGCCCGAAGTGCAGCTCGTCGAGAGCGGAGG |
| | | GGGACTGGTGCAGCCCGGAGGAAGCCTGAGGCTGTCCTGCGCTG |
| | | CCTCCGGCTACACCTTCACCTCCTACTGGATGAACTGGGTCAGAC |
| | | AGGCACCTGGAAAGGGACTGGTCTGGGTGTCGCGCATTGACCCC |
| | | TACGACTCCGAAACCCATTACAATCAGAAATTCAAGGACCGCTT |
| | | CACCATCTCCGTGGACAAAGCCAAGAGCACCGCGTACCTCCAAA |
| | | TGAACTCCCTGCGCGCTGAGGATACAGCAGTGTACTATTGCGCC |
| | | CGGGGAAACTGGGATGATTACTGGGGCCAGGGAACTACTGTGAC |
| | | TGTGTCATCCGGGGGTGGCGGTAGCGGAGGAGGGGGCTCCGGCG |
| | | GCGGCGGCTCAGGGGGCGGAGGAAGCGACGTCGTGATGACCCA |
| | | GTCACCGGCATTCCTGTCCGTGACTCCCGGAGAAAAGGTCACGA |
| | | TTACTTGCCGGGCGTCCAAGAGCATCTCCAAGGACCTCGCCTGGT |
| | | ACCAACAGAAGCCGGACCAGGCCCCTAAGCTGTTGATCTACTCG |
| | | GGGTCCACCCTTCAATCGGGAGTGCCATCGCGGTTTAGCGGTTCG |
| | | GGTTCTGGGACCGACTTCACTTTCACCATCTCCTCACTGGAAGCC |
| | | GAGGATGCCGCCACTTACTACTGTCAGCAGCACAACAAGTATCC |
| | | GTACACCTTCGGAGGCGGTACCAAAGTGGAGATCAAGACCACTA |
| | | CCCCAGCACCGAGGCCACCCACCCCGGCTCCTACCATCGCCTCCC |
| | | AGCCTCTGTCCCTGCGTCCGGAggcatgtagacccgcagctggtggggccgtgcata |
| | | cccggggtcttgacttcgcctgcgatatctacatttgggcccctctggctggtacttgcggggtcctgctgcttt |
| | | cactcgtgatcactctttactgtaagcgcggtcggaagaagctgctgtacatctttaagcaacccttcatgagg |
| | | cctgtgcagactactcaagaggaggacggctgttcatgccggttcccagaggaggaggaaggcggctgc |
| | | gaactgcgcgtgaaattcagccgcagcgcagatgctccagcctacaagcaggggcagaaccagctctac |
| | | aacgaactcaatcttggtcggagagaggagtacgacgtgctggacaagcggagaggacgggacccaga |
| | | aatgggcgggaagccgcgcagaaagaatccccaagagggcctgtacaacgagctccaaaaggataaga |
| | | tggcagaagcctatagcgagattggtatgaaaggggaacgcagaagaggcaaaggccacgacggactgt |
| | | accagggactcagcaccgccaccaaggacacctatgacgctcttcacatgcaggccctgccgcctcgg |
| hzCAR123- | 151 | MALPVTALLLPLALLLHAARPEVQLVESGGGLVQPGGSLRLSCAAS |
| 27 AA | | GYTFTSYWMNWVRQ |
| | | APGKGLVWVSRIDPYDSETHYNQKFKDRFTISVDKAKSTAYLQMNS |
| | | LRAEDTAVYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 210 | MALPVTALLLPLALLLHAARPEVQLVESGGGLVQPGGSLRLSCAAS |
| 27 | | GYTFTSYWMNWVRQ |
| scFv | | APGKGLVWVSRIDPYDSETHYNQKFKDRFTISVDKAKSTAYLQMNS |
| | | LRAEDTAVYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIK |
| hzCAR123- | 269 | EVQLVESGGGLVQPGGSLRLSCAASGYTFTSYWMNWVRQAPGKG |
| 27 VH | | LVWVSRIDPYDSETHYNQKFKDRFTISVDKAKSTAYLQMNSLRAED |
| | | TAVYYCARGNWDDYWGQGTTVTVSS |
| hzCAR123- | 328 | DVVMTQSPAFLSVTPGEKVTITCRASKSISKDLAWYQQKPDQAPKL |
| 27 VL | | LIYSGSTLQSGVPSRFSGSGSGTDFTFTISSLEAEDAATYYCQQHNKY |
| | | PYTFGGGTKVEIK |
| hzCAR123- | 93 | ATGGCCCTCCCTGTCACCGCCCTGCTGCTTCCGCTGGCTCTTCTG |
| 28 NT | | CTCCACGCCGCTCGGCCCGAAGTGCAGCTCGTCGAGAGCGGAGG |
| | | GGGACTGGTGCAGCCCGGAGGAAGCCTGAGGCTGTCCTGCGCTG |
| | | CCTCCGGCTACACCTTCACCTCCTACTGGATGAACTGGGTCAGAC |
| | | AGGCACCTGGAAAGGGACTGGTCTGGGTGTCGCGCATTGACCCC |
| | | TACGACTCCGAAACCCATTACAATCAGAAATTCAAGGACCGCTT |
| | | CACCATCTCCGTGGACAAAGCCAAGAGCACCGCGTACCTCCAAA |
| | | TGAACTCCCTGCGCGCTGAGGATACAGCAGTGTACTATTGCGCC |
| | | CGGGGAAACTGGGATGATTACTGGGGCCAGGGAACTACTGTGAC |
| | | TGTGTCATCCGGGGGTGGCGGTAGCGGAGGAGGGGGCTCCGGCG |
| | | GCGGCGGCTCAGGGGGCGGAGGAAGCGACGTGGTCATGACTCA |
| | | GTCCCCGGACTCACTCGCGGTGTCGCTTGGAGAGAGAGCGACCA |
| | | TCAACTGTCGGGCCTCAAAGAGCATCAGCAAGGACCTGGCCTGG |
| | | TACCAGCAGAAGCCGGGACAGCCGCCAAAGCTGCTGATCTACTC |
| | | CGGGTCCACCTTGCAATCTGGTGTCCCTGACCGGTTCTCCGGTTC |
| | | CGGGTCGGGTACCGACTTCACGCTCACTATTTCGTCGCTGCAAGC |
| | | CGAAGATGTGGCCGTGTACTATTGCCAACAGCACAACAAGTACC |
| | | CCTACACTTTTGGCGGAGGCACCAAGGTGGAAATCAAGACCACT |
| | | ACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 152 | MALPVTALLLPLALLLHAARPEVQLVESGGGLVQPGGSLRLSCAAS |
| 28 AA | | GYTFTSYWMNWVRQ |
| | | APGKGLVWVSRIDPYDSETHYNQKFKDRFTISVDKAKSTAYLQMNS |
| | | LRAEDTAVYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIKTTTPAPRPPTPAPTIASQPLSLRPE |
| | | ACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 211 | MALPVTALLLPLALLLHAARPEVQLVESGGGLVQPGGSLRLSCAAS |
| 28 | | GYTFTSYWMNWVRQ |
| scFv | | APGKGLVWVSRIDPYDSETHYNQKFKDRFTISVDKAKSTAYLQMNS |
| | | LRAEDTAVYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIK |
| hzCAR123- | 270 | EVQLVESGGGLVQPGGSLRLSCAASGYTFTSYWMNWVRQAPGKG |
| 28 VH | | LVWVSRIDPYDSETHYNQKFKDRFTISVDKAKSTAYLQMNSLRAED |
| | | TAVYYCARGNWDDYWGQGTTVTVSS |
| hzCAR123- | 329 | DVVMTQSPDSLAVSLGERATINCRASKSISKDLAWYQQKPGQPPKL |
| 28 VL | | LIYSGSTLQSGVPDRFSGSGSGTDFTLTISSLQAEDVAVYYCQQHNK |
| | | YPYTFGGGTKVEIK |
| hzCAR123- | 94 | ATGGCCCTCCCTGTCACCGCCCTGCTGCTTCCGCTGGCTCTTCTG |
| 29 NT | | CTCCACGCCGCTCGGCCCGACGTGCAGCTCACCCAGTCGCCCTCA |
| | | TTTCTGTCGGCCTCAGTGGGAGACAGAGTGACCATTACTTGTCGG |
| | | GCCTCCAAGAGCATCTCCAAGGACCTGGCCTGGTATCAGCAGAA |
| | | GCCAGGAAAGGCGCCTAAGTTGCTCATCTACTCGGGGTCGACCC |
| | | TGCAATCTGGCGTGCCGTCCCGGTTCTCCGGTTCGGGAAGCGGTA |
| | | CCGAATTCACCCTTACTATCTCCTCCCTGCAACCGGAGGACTTCG |
| | | CCACCTACTACTGCCAACAGCACAACAAGTACCCGTACACTTTC |
| | | GGGGGTGGCACGAAGGTCGAAATCAAGGGGGGTGGCGGTAGCG |
| | | GAGGAGGGGGCTCCGGCGGCGGCGGCTCAGGGGGCGGAGGAAG |
| | | CGAAGTGCAGCTCGTCGAGAGCGGAGGGGGACTGGTGCAGCCC |
| | | GGAGGAAGCCTGAGGCTGTCCTGCGCTGCCTCCGGCTACACCTT |
| | | CACCTCCTACTGGATGAACTGGGTCAGACAGGCACCTGGAAAGG |
| | | GACTGGTCTGGGTGTCGCGCATTGACCCCTACGACTCCGAAACC |
| | | CATTACAATCAGAAATTCAAGGACCGCTTCACCATCTCCGTGGA |
| | | CAAAGCCAAGAGCACCGCGTACCTCCAAATGAACTCCCTGCGCG |
| | | CTGAGGATACAGCAGTGTACTATTGCGCCCGGGGAAACTGGGAT |
| | | GATTACTGGGGCCAGGGAACTACTGTGACTGTGTCATCCACCAC |
| | | TACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 153 | MALPVTALLLPLALLLHAARPDVQLTQSPSFLSASVGDRVTITCRAS |
| 29 AA | | KSISKDLAWYQQK |
| | | PGKAPKLLIYSGSTLQSGVPSRFSGSGSGTEFTLTISSLQPEDFATYYC |
| | | QQHNKYPYTFG |
| | | GGTKVEIKGGGGSGGGGSGGGGSGGGGSEVQLVESGGGLVQPGGS |
| | | LRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 212 | MALPVTALLLPLALLLHAARPDVQLTQSPSFLSASVGDRVTITCRAS |
| 29 | | KSISKDLAWYQQK |
| scFv | | PGKAPKLLIYSGSTLQSGVPSRFSGSGSGTEFTLTISSLQPEDFATYYC |
| | | QQHNKYPYTFG |
| | | GGTKVEIKGGGGSGGGGSGGGGSGGGGSEVQLVESGGGLVQPGGS |
| | | LRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSS |
| hzCAR123- | 271 | EVQLVESGGGLVQPGGSLRLSCAASGYTFTSYWMNWVRQAPGKG |
| 29 VH | | LVWVSRIDPYDSETHYNQKFKDRFTISVDKAKSTAYLQMNSLRAED |
| | | TAVYYCARGNWDDYWGQGTTVTVSS |
| hzCAR123- | 330 | DVQLTQSPSFLSASVGDRVTITCRASKSISKDLAWYQQKPGKAPKLL |
| 29 VL | | IYSGSTLQSGVPSRFSGSGSGTEFTLTISSLQPEDFATYYCQQHNKYP |
| | | YTFGGGTKVEIK |
| hzCAR123- | 95 | ATGGCCCTCCCTGTCACCGCCCTGCTGCTTCCGCTGGCTCTTCTG |
| 30 NT | | CTCCACGCCGCTCGGCCCGAAGTGGTGCTGACCCAGTCGCCCGC |
| | | AACCCTCTCTCTGTCGCCGGGAGAACGCGCCACTCTTTCCTGTCG |
| | | GGCGTCCAAGAGCATCTCAAAGGACCTCGCCTGGTACCAGCAGA |
| | | AGCCTGGTCAAGCCCCGCGGCTGCTGATCTACTCCGGCTCCACGC |
| | | TGCAATCAGGAATCCCAGCCAGATTTTCCGGTTCGGGGTCGGGG |
| | | ACTGACTTCACCTTGACCATTAGCTCGCTGGAACCTGAGGACTTC |
| | | GCCGTGTATTACTGCCAGCAGCACAACAAGTACCCGTACACCTT |
| | | CGGAGGCGGTACTAAGGTCGAGATCAAGGGGGGTGGCGGTAGC |
| | | GGAGGAGGGGGCTCCGGCGGCGGCGGCTCAGGGGGCGGAGGAA |
| | | GCGAAGTGCAGCTCGTCGAGAGCGGAGGGGGACTGGTGCAGCC |
| | | CGGAGGAAGCCTGAGGCTGTCCTGCGCTGCCTCCGGCTACACCT |
| | | TCACCTCCTACTGGATGAACTGGGTCAGACAGGCACCTGGAAAG |
| | | GGACTGGTCTGGGTGTCGCGCATTGACCCCTACGACTCCGAAAC |
| | | CCATTACAATCAGAAATTCAAGGACCGCTTCACCATCTCCGTGG |
| | | ACAAAGCCAAGAGCACCGCGTACCTCCAAATGAACTCCCTGCGC |
| | | GCTGAGGATACAGCAGTGTACTATTGCGCCCGGGGAAACTGGGA |
| | | TGATTACTGGGGCCAGGGAACTACTGTGACTGTGTCATCCACCA |
| | | CTACCCCAGCACCGAGGCCACCCACCCCGGCTCCTACCATCGCCT |
| | | CCCAGCCTCTGTCCCTGCGTCCGGAggcatgtagacccgcagctggtggggccgtg |
| | | catacccggggtcttgacttcgcctgcgatatctacatttgggcccctctggctggtacttgcggggtcctgct |
| | | gctttcactcgtgatcactctttactgtaagcgcggtcggaagaagctgctgtacatctttaagcaacccttcat |
| | | gaggcctgtgcagactactcaagaggaggacggctgttcatgccggttcccagaggaggaggaaggcgg |
| | | ctgcgaactgcgcgtgaaattcagccgcagcgcagatgctccagcctacaagcaggggcagaaccagct |
| | | ctacaacgaactcaatcttggtcggagagaggagtacgacgtgctggacaagcggagaggacgggaccc |
| | | agaaatgggcgggaagccgcgcagaaagaatccccaagagggcctgtacaacgagctccaaaaggata |
| | | agatggcagaagcctatagcgagattggtatgaaaggggaacgcagaagaggcaaaggccacgacgga |
| | | ctgtaccagggactcagcaccgccaccaaggacacctatgacgctcttcacatgcaggccctgccgcctcgg |
| hzCAR123- | 154 | MALPVTALLLPLALLLHAARPEVVLTQSPATLSLSPGERATLSCRAS |
| 30 AA | | KSISKDLAWYQQK |
| | | PGQAPRLLIYSGSTLQSGIPARFSGSGSGTDFTLTISSLEPEDFAVYYC |
| | | QQHNKYPYTFG |
| | | GGTKVEIKGGGGSGGGGSGGGGSGGGGSEVQLVESGGGLVQPGGS |
| | | LRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 213 | MALPVTALLLPLALLLHAARPEVVLTQSPATLSLSPGERATLSCRAS |
| 30 | | KSISKDLAWYQQK |
| scFv | | PGQAPRLLIYSGSTLQSGIPARFSGSGSGTDFTLTISSLEPEDFAVYYC |
| | | QQHNKYPYTFG |
| | | GGTKVEIKGGGGSGGGGSGGGGSGGGGSEVQLVESGGGLVQPGGS |
| | | LRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSS |
| hzCAR123- | 272 | EVQLVESGGGLVQPGGSLRLSCAASGYTFTSYWMNWVRQAPGKG |
| 30 VH | | LVWVSRIDPYDSETHYNQKFKDRFTISVDKAKSTAYLQMNSLRAED |
| | | TAVYYCARGNWDDYWGQGTTVTVSS |
| hzCAR123- | 331 | EVVLTQSPATLSLSPGERATLSCRASKSISKDLAWYQQKPGQAPRLL |
| 30 VL | | IYSGSTLQSGIPARFSGSGSGTDFTLTISSLEPEDFAVYYCQQHNKYP |
| | | YTFGGGTKVEIK |
| hzCAR123- | 96 | ATGGCCCTCCCTGTCACCGCCCTGCTGCTTCCGCTGGCTCTTCTG |
| 31 NT | | CTCCACGCCGCTCGGCCCGACGTCGTGATGACCCAGTCACCGGC |
| | | ATTCCTGTCCGTGACTCCCGGAGAAAAGGTCACGATTACTTGCCG |
| | | GGCGTCCAAGAGCATCTCCAAGGACCTCGCCTGGTACCAACAGA |
| | | AGCCGGACCAGGCCCCTAAGCTGTTGATCTACTCGGGGTCCACC |
| | | CTTCAATCGGGAGTGCCATCGCGGTTTAGCGGTTCGGGTTCTGGG |
| | | ACCGACTTCACTTTCACCATCTCCTCACTGGAAGCCGAGGATGCC |
| | | GCCACTTACTACTGTCAGCAGCACAACAAGTATCCGTACACCTTC |
| | | GGAGGCGGTACCAAAGTGGAGATCAAGGGGGGTGGCGGTAGCG |
| | | GAGGAGGGGGCTCCGGCGGCGGCGGCTCAGGGGGCGGAGGAAG |
| | | CGAAGTGCAGCTCGTCGAGAGCGGAGGGGGACTGGTGCAGCCC |
| | | GGAGGAAGCCTGAGGCTGTCCTGCGCTGCCTCCGGCTACACCTT |
| | | CACCTCCTACTGGATGAACTGGGTCAGACAGGCACCTGGAAAGG |
| | | GACTGGTCTGGGTGTCGCGCATTGACCCCTACGACTCCGAAACC |
| | | CATTACAATCAGAAATTCAAGGACCGCTTCACCATCTCCGTGGA |
| | | CAAAGCCAAGAGCACCGCGTACCTCCAAATGAACTCCCTGCGCG |
| | | CTGAGGATACAGCAGTGTACTATTGCGCCCGGGGAAACTGGGAT |
| | | GATTACTGGGGCCAGGGAACTACTGTGACTGTGTCATCCACCAC |
| | | TACCCCAGCACCGAGGCCACCCACCCCGGCTCCTACCATCGCCTC |
| | | CCAGCCTCTGTCCCTGCGTCCGGAggcatgtagacccgcagctggtggggccgtgc |
| | | atacccggggtcttgacttcgcctgcgatatctacatttgggcccctctggctggtacttgcggggtcctgctg |
| | | ctttcactcgtgatcactctttactgtaagcgcggtcggaagaagctgctgtacatctttaagcaacccttcatg |
| | | aggcctgtgcagactactcaagaggaggacggctgttcatgccggttcccagaggaggaggaaggcggc |
| | | tgcgaactgcgcgtgaaattcagccgcagcgcagatgctccagcctacaagcaggggcagaaccagctct |
| | | acaacgaactcaatcttggtcggagagaggagtacgacgtgctggacaagcggagaggacgggaccca |
| | | gaaatgggcgggaagccgcgcagaaagaatccccaagagggcctgtacaacgagctccaaaaggataa |
| | | gatggcagaagcctatagcgagattggtatgaaaggggaacgcagaagaggcaaaggccacgacggac |
| | | tgtaccagggactcagcaccgccaccaaggacacctatgacgctcttcacatgcaggccctgccgcctcgg |
| hzCAR123- | 155 | MALPVTALLLPLALLLHAARPDVVMTQSPAFLSVTPGEKVTITCRAS |
| 31 AA | | KSISKDLAWYQQK |
| | | PDQAPKLLIYSGSTLQSGVPSRFSGSGSGTDFTFTISSLEAEDAATYY |
| | | CQQHNKYPYTFG |
| | | GGTKVEIKGGGGSGGGGSGGGGSGGGGSEVQLVESGGGLVQPGGS |
| | | LRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 214 | MALPVTALLLPLALLLHAARPDVVMTQSPAFLSVTPGEKVTITCRAS |
| 31 | | KSISKDLAWYQQK |
| scFv | | PDQAPKLLIYSGSTLQSGVPSRFSGSGSGTDFTFTISSLEAEDAATYY |
| | | CQQHNKYPYTFG |
| | | GGTKVEIKGGGGSGGGGSGGGGSGGGGSEVQLVESGGGLVQPGGS |
| | | LRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSS |
| hzCAR123- | 273 | EVQLVESGGGLVQPGGSLRLSCAASGYTFTSYWMNWVRQAPGKG |
| 31 VH | | LVWVSRIDPYDSETHYNQKFKDRFTISVDKAKSTAYLQMNSLRAED |
| | | TAVYYCARGNWDDYWGQGTTVTVSS |
| hzCAR123- | 332 | DVVMTQSPAFLSVTPGEKVTITCRASKSISKDLAWYQQKPDQAPKL |
| 31 VL | | LIYSGSTLQSGVPSRFSGSGSGTDFTFTISSLEAEDAATYYCQQHNKY |
| | | PYTFGGGTKVEIK |
| hzCAR123- | 97 | ATGGCCCTCCCTGTCACCGCCCTGCTGCTTCCGCTGGCTCTTCTG |
| 32 NT | | CTCCACGCCGCTCGGCCCGACGTGGTCATGACTCAGTCCCCGGA |
| | | CTCACTCGCGGTGTCGCTTGGAGAGAGAGCGACCATCAACTGTC |
| | | GGGCCTCAAAGAGCATCAGCAAGGACCTGGCCTGGTACCAGCAG |
| | | AAGCCGGGACAGCCGCCAAAGCTGCTGATCTACTCCGGGTCCAC |
| | | CTTGCAATCTGGTGTCCCTGACCGGTTCTCCGGTTCCGGGTCGGG |
| | | TACCGACTTCACGCTCACTATTTCGTCGCTGCAAGCCGAAGATGT |
| | | GGCCGTGTACTATTGCCAACAGCACAACAAGTACCCCTACACTTT |
| | | TGGCGGAGGCACCAAGGTGGAAATCAAGGGGGGTGGCGGTAGC |
| | | GGAGGAGGGGGCTCCGGCGGCGGCGGCTCAGGGGGCGGAGGAA |
| | | GCGAAGTGCAGCTCGTCGAGAGCGGAGGGGGACTGGTGCAGCC |
| | | CGGAGGAAGCCTGAGGCTGTCCTGCGCTGCCTCCGGCTACACCT |
| | | TCACCTCCTACTGGATGAACTGGGTCAGACAGGCACCTGGAAAG |
| | | GGACTGGTCTGGGTGTCGCGCATTGACCCCTACGACTCCGAAAC |
| | | CCATTACAATCAGAAATTCAAGGACCGCTTCACCATCTCCGTGG |
| | | ACAAAGCCAAGAGCACCGCGTACCTCCAAATGAACTCCCTGCGC |
| | | GCTGAGGATACAGCAGTGTACTATTGCGCCCGGGGAAACTGGGA |
| | | TGATTACTGGGGCCAGGGAACTACTGTGACTGTGTCATCCACCA |
| | | CTACCCCAGCACCGAGGCCACCCACCCCGGCTCCTACCATCGCCT |
| | | CCCAGCCTCTGTCCCTGCGTCCGGAggcatgtagacccgcagctggtggggccgtg |
| | | catacccggggtcttgacttcgcctgcgatatctacatttgggcccctctggctggtacttgcggggtcctgct |
| | | gctttcactcgtgatcactctttactgtaagcgcggtcggaagaagctgctgtacatctttaagcaacccttcat |
| | | gaggcctgtgcagactactcaagaggaggacggctgttcatgccggttcccagaggaggaggaaggcgg |
| | | ctgcgaactgcgcgtgaaattcagccgcagcgcagatgctccagcctacaagcaggggcagaaccagct |
| | | ctacaacgaactcaatcttggtcggagagaggagtacgacgtgctggacaagcggagaggacgggaccc |
| | | agaaatgggcgggaagccgcgcagaaagaatccccaagagggcctgtacaacgagctccaaaaggata |
| | | agatggcagaagcctatagcgagattggtatgaaaggggaacgcagaagaggcaaaggccacgacgga |
| | | ctgtaccagggactcagcaccgccaccaaggacacctatgacgctcttcacatgcaggccctgccgcctcgg |
| hzCAR123- | 156 | MALPVTALLLPLALLLHAARPDVVMTQSPDSLAVSLGERATINCRA |
| 32 AA | | SKSISKDLAWYQQK |
| | | PGQPPKLLIYSGSTLQSGVPDRFSGSGSGTDFTLTISSLQAEDVAVYY |
| | | CQQHNKYPYTFG |
| | | GGTKVEIKGGGGSGGGGSGGGGSGGGGSEVQLVESGGGLVQPGGS |
| | | LRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSSTTTPAPRPPTPAPTIASQPLSLR |
| | | PEACRPAAGGAVHT |
| | | RGLDFACDIYIWAPLAGTCGVLLLSLVITLYCKRGRKKLLYIFKQPF |
| | | MRPVQTTQEEDGC |
| | | SCRFPEEEEGGCELRVKFSRSADAPAYKQGQNQLYNELNLGRREEY |
| | | DVLDKRRGRDPEMG |
| | | GKPRRKNPQEGLYNELQKDKMAEAYSEIGMKGERRRGKGHDGLY |
| | | QGLSTATKDTYDALHM |
| | | QALPPR |
| hzCAR123- | 215 | MALPVTALLLPLALLLHAARPDVVMTQSPDSLAVSLGERATINCRA |
| 32 | | SKSISKDLAWYQQK |
| scFv | | PGQPPKLLIYSGSTLQSGVPDRFSGSGSGTDFTLTISSLQAEDVAVYY |
| | | CQQHNKYPYTFG |
| | | GGTKVEIKGGGGSGGGGSGGGGSGGGGSEVQLVESGGGLVQPGGS |
| | | LRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSS |
| hzCAR123- | 274 | EVQLVESGGGLVQPGGSLRLSCAASGYTFTSYWMNWVRQAPGKG |
| 32 VH | | LVWVSRIDPYDSETHYNQKFKDRFTISVDKAKSTAYLQMNSLRAED |
| | | TAVYYCARGNWDDYWGQGTTVTVSS |
| hzCAR123- | 333 | DVVMTQSPDSLAVSLGERATINCRASKSISKDLAWYQQKPGQPPKL |
| 32 VL | | LIYSGSTLQSGVPDRFSGSGSGTDFTLTISSLQAEDVAVYYCQQHNK |
| | | YPYTFGGGTKVEIK |

In embodiments, a CAR molecule described herein comprises a scFv that specifically binds to CD123, and does not contain a leader sequence, e.g., the amino acid sequence SEQ ID NO: 1. Table 12B below provides amino acid and nucleotide sequences for CD123 scFv sequences that do not contain a leader sequence SEQ ID NO: 1.

TABLE 12B
CD123 CAR scFv sequences

| | SEQ | |
|---|---|---|
| Name | ID | Sequence |
| CAR123-2 | 479 | CAAGTGCAACTCGTCCAAAGCGGAGCGGAAGTCAAGAAACCCG |
| scFv-NT | | GAGCGAGCGTGAAAGTGTCCTGCAAAGCCTCCGGCTACACCTTT |
| | | ACGGGCTACTACATGCACTGGGTGCGCCAGGCACCAGGACAGG |
| | | GTCTTGAATGGATGGGATGGATCAACCCTAATTCGGGCGGAACT |
| | | AACTACGCACAGAAGTTCCAGGGGAGAGTGACTCTGACTCGGG |
| | | ATACCTCCATCTCAACTGTCTACATGGAACTCTCCCGCTTGCGGT |
| | | CAGATGATACGGCAGTGTACTACTGCGCCCGCGACATGAATATC |
| | | CTGGCTACCGTGCCGTTCGACATCTGGGGACAGGGGACTATGGT |
| | | TACTGTCTCATCGGGCGGTGGAGGTTCAGGAGGAGGCGGCTCG |
| | | GGAGGCGGAGGTTCGGACATTCAGATGACCCAGTCCCCATCCTC |
| | | TCTGTCGGCCAGCGTCGGAGATAGGGTGACCATTACCTGTCGGG |
| | | CCTCGCAAAGCATCTCCTCGTACCTCAACTGGTATCAGCAAAAG |
| | | CCGGGAAAGGCGCCTAAGCTGCTGATCTACGCCGCTTCGAGCTT |
| | | GCAAAGCGGGGTGCCATCCAGATTCTCGGGATCAGGCTCAGGA |
| | | ACCGACTTCACCCTGACCGTGAACAGCCTCCAGCCGGAGGACTT |
| | | TGCCACTTACTACTGCCAGCAGGGAGACTCCGTGCCGCTTACTT |
| | | TCGGGGGGGGTACCCGCCTGGAGATCAAG |
| CAR123-2 | 480 | QVQLVQSGAEVKKPGASVKVSCKASGYTFTGYYMHWVRQAPGQ |
| scFv-AA | | GLEWMGWINPNSGGTNYAQKFQGRVTLTRDTSISTVYMELSRLRS |
| | | DDTAVYYCARDMNILATVPFDIWGQGTMVTVSSGGGGSGGGGSG |
| | | GGGSDIQMTQSPSSLSASVGDRVTITCRASQSISSYLNWYQQKPGK |
| | | APKLLIYAASSLQSGVPSRFSGSGSGTDFTLTVNSLQPEDFATYYCQ |
| | | QGDSVPLTFGGGTRLEIK |
| CAR123-2 | 481 | atggccctccctgtcaccgccctgctgcttccgctggctcttctgctccacgccgctcggccccaagtgcaa |
| ORF-free | | ctcgtccaaagcggagcggaagtcaagaaacccggagcgagcgtgaaagtgtcctgcaaagcctccgg |
| NT | | ctacacctttacgggctactacatgcactgggtgcgccaggcaccaggacagggtcttgaatggatggga |
| | | tggatcaaccctaattcgggcggaactaactacgcacagaagttccaggggagagtgactctgactcggg |
| | | atacctccatctcaactgtctacatggaactctcccgcttgcggtcagatgatacggcagtgtactactgcgc |
| | | ccgcgacatgaatatcctggctaccgtgccgttcgacatctggggacaggggactatggttactgtctcatc |
| | | gggcggtggaggttcaggaggaggcggctcgggaggcggaggttcggacattcagatgacccagtcc |
| | | ccatcctctctgtcggccagcgtcggagatagggtgaccattacctgtcgggcctcgcaaagcatctcctc |
| | | gtacctcaactggtatcagcaaaagccgggaaaggcgcctaagctgctgatctacgccgcttcgagcttg |
| | | caaagcggggtgccatccagattctcgggatcaggctcaggaaccgacttcaccctgaccgtgaacagc |
| | | ctccagccggaggactttgccacttactactgccagcagggagactccgtgccgcttactttcggggggg |
| | | gtacccgcctggagatcaagaccactaccccagcaccgaggccacccaccccggctcctaccatcgcct |
| | | cccagcctctgtccctgcgtccggaggcatgtagacccgcagctggtggggccgtgcatacccggggtc |
| | | ttgacttcgcctgcgatatctacatttgggcccctctggctggtacttgcggggtcctgctgctttcactcgtg |
| | | atcactctttactgtaagcgcggtcggaagaagctgctgtacatctttaagcaacccttcatgaggcctgtgc |
| | | agactactcaagaggaggacggctgttcttgccggttcccagaggaggaggaaggcggctgcgaactg |
| | | cgcgtgaaattcagccgcagcgcagacgctccagcctacaagcaggggcagaaccagctctacaacga |
| | | actcaatcttggtcggagagaggagtacgacgtgctggacaagcggagaggacgggacccagaaatgg |
| | | gcgggaagccgcgcagaaagaatccccaagagggcctgtacaacgagctccaaaaggataagatggc |
| | | agaagcctatagcgagattggtatgaaaggggaacgcagaagaggcaaaggccacgacggactgtacc |
| | | agggactcagcaccgccaccaaggacacctatgacgctcttcacatgcaggccctgccgcctcggtaagt |
| | | cgacagctcgctttcttgctgtccaatttctattaaaggttcctttgttccctaagtccaactactaaactggggg |
| | | atattatgaagggccttgagcatctggattctgcctaataaaaaacatttattttcattgctgcgtcgagagctc |
| | | gctttcttgctgtccaatttctattaaaggttcctttgttccctaagtccaactactaaactgggggatattatgaa |
| | | gggccttgagcatctggattctgcctaataaaaaacatttattttcattgctgcctcgacgaattc |
| CAR123-3 | 482 | CAAGTCCAACTCGTTCAATCCGGCGCAGAAGTCAAGAAGCCAG |
| scFv-NT | | GAGCATCAGTGAAAGTGTCCTGCAAAGCCTCAGGCTACATCTTC |
| | | ACGGGATACTACATCCACTGGGTGCGCCAGGCTCCGGGCCAGG |
| | | GCCTTGAGTGGATGGGCTGGATCAACCCTAACTCTGGGGGAACC |
| | | AACTACGCTCAGAAGTTCCAGGGGAGGGTCACTATGACTCGCG |
| | | ATACCTCCATCTCCACTGCGTACATGGAACTCTCGGGACTGAGA |
| | | TCCGACGATCCTGCCGTGTACTACTGCGCCCGGGACATGAACAT |
| | | CTTGGCGACCGTGCCGTTTGACATTTGGGGACAGGGCACCCTCG |
| | | TCACTGTGTCGAGCGGTGGAGGAGGCTCGGGGGGTGGCGGATC |
| | | AGGAGGGGGAGGAAGCGACATCCAGCTGACTCAGAGCCCATCG |
| | | TCGTTGTCCGCGTCGGTGGGGGATAGAGTGACCATTACTTGCCG |
| | | CGCCAGCCAGAGCATCTCATCATATCTGAATTGGTACCAGCAGA |
| | | AGCCCGGAAAGGCCCCAAAACTGCTGATCTACGCTGCAAGCAG |
| | | CCTCCAATCGGGAGTGCCGTCACGGTTCTCCGGGTCCGGTTCGG |
| | | GAACTGACTTTACCCTGACCGTGAATTCGCTGCAACCGGAGGAT |
| | | TTCGCCACGTACTACTGTCAGCAAGGAGACTCCGTGCCGCTGAC |
| | | CTTCGGTGGAGGCACCAAGGTCGAAATCAAG |
| CAR123-3 | 483 | QVQLVQSGAEVKKPGASVKVSCKASGYIFTGYYIHWVRQAPGQGL |
| scFv-AA | | EWMGWINPNSGGTNYAQKFQGRVTMTRDTSISTAYMELSGLRSD |
| | | DPAVYYCARDMNILATVPFDIWGQGTLVTVSSGGGGSGGGGSGG |
| | | GGSDIQLTQSPSSLSASVGDRVTITCRASQSISSYLNWYQQKPGKAP |
| | | KLLIYAASSLQSGVPSRFSGSGSGTDFTLTVNSLQPEDFATYYCQQG |
| | | DSVPLTFGGGTKVEIK |
| CAR123-4 | 484 | CAAGTCCAACTCCAACAGTCAGGCGCAGAAGTGAAAAAGAGCG |
| scFv-NT | | GTGCATCGGTGAAAGTGTCATGCAAAGCCTCGGGCTACACCTTC |
| | | ACTGACTACTATATGCACTGGCTGCGGCAGGCACCGGGACAGG |
| | | GACTTGAGTGGATGGGATGGATCAACCCGAATTCAGGGGACAC |
| | | TAACTACGCGCAGAAGTTCCAGGGGAGAGTGACCCTGACGAGG |
| | | GACACCTCAATTTCGACCGTCTACATGGAATTGTCGCGCCTGAG |
| | | ATCGGACGATACTGCTGTGTACTACTGTGCCCGCGACATGAACA |
| | | TCCTCGCGACTGTGCCTTTTGATATCTGGGGACAGGGGACTATG |
| | | GTCACCGTTTCCTCCGCTTCCGGTGGCGGAGGCTCGGGAGGCCG |
| | | GGCCTCCGGTGGAGGAGGCAGCGACATCCAGATGACTCAGAGC |
| | | CCTTCCTCGCTGAGCGCCTCAGTGGGAGATCGCGTGACCATCAC |
| | | TTGCCGGGCCAGCCAGTCCATTTCGTCCTACCTCAATTGGTACC |
| | | AGCAGAAGCCGGGAAAGGCGCCCAAGCTCTTGATCTACGCTGC |
| | | GAGCTCCCTGCAAAGCGGGGTGCCGAGCCGATTCTCGGGTTCCG |
| | | GCTCGGGAACCGACTTCACTCTGACCATCTCATCCCTGCAACCA |
| | | GAGGACTTTGCCACCTACTACTGCCAACAAGGAGATTCTGTCCC |
| | | ACTGACGTTCGGCGGAGGAACCAAGGTCGAAATCAAG |
| CAR123-4 | 485 | QVQLQQSGAEVKKSGASVKVSCKASGYTFTDYYMHWLRQAPGQ |
| scFv-AA | | GLEWMGWINPNSGDTNYAQKFQGRVTLTRDTSISTVYMELSRLRS |
| | | DDTAVYYCARDMNILATVPFDIWGQGTMVTVSSASGGGGSGGRA |
| | | SGGGGSDIQMTQSPSSLSASVGDRVTITCRASQSISSYLNWYQQKP |
| | | GKAPKLLIYAASSLQSGVPSRFSGSGSGTDFTLTISSLQPEDFATYYC |
| | | QQGDSVPLTFGGGTKVEIK |
| CAR123-1 | 478 | QVQLVQSGAEVKKPGASVKVSCKASGYTFTGYYMHWVRQAPGQ |
| scFv-AA | | GLEWMGWINPNSGGTNYAQKFQGRVTMTRDTSISTAYMELSRLRS |
| | | DDTAVYYCARDMNILATVPFDIWGQGTMVTVSSGGGGSGGGGSG |
| | | GGGSDIQMTQSPSSLSASVGDRVTITCRASQSISTYLNWYQQKPGK |
| | | APNLLIYAAFSLQSGVPSRFSGSGSGTDFTLTINSLQPEDFATYYCQ |
| | | QGDSVPLTFGGGTKLEIK |
| hzCAR123-1 | 556 | QVQLVQSGAEVKKPGASVKVSCKASGYTFTSYWMNWVRQAPGQ |
| scFv | | GLEWMGRIDPYDSETHYNQKFKDRVTMTVDKSTSTAYMELSSLRS |
| | | EDTAVYYCARGNWDDYWGQGTTVTVSSGGGGSGGGGSGGGGSG |
| | | GGGSDVQLTQSPSFLSASVGDRVTITCRASKSISKDLAWYQQKPGK |
| | | APKLLIYSGSTLQSGVPSRFSGSGSGTEFTLTISSLQPEDFATYYCQQ |
| | | HNKYPYTFGGGTKVEIK |
| hzCAR123-2 | 557 | QVQLVQSGAEVKKPGASVKVSCKASGYTFTSYWMNWVRQ |
| scFv | | APGQGLEWMGRIDPYDSETHYNQKFKDRVTMTVDKSTSTAYMEL |
| | | SSLRSEDTAVYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDF |
| | | TLTISSLEPEDFA |
| | | VYYCQQHNKYPYTFGGGTKVEIK |
| hzCAR123-3 | 558 | QVQLVQSGAEVKKPGASVKVSCKASGYTFTSYWMNWVRQ |
| scFv | | APGQGLEWMGRIDPYDSETHYNQKFKDRVTMTVDKSTSTAYMEL |
| | | SSLRSEDTAVYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIK |
| hzCAR123-4 | 559 | QVQLVQSGAEVKKPGASVKVSCKASGYTFTSYWMNWVRQ |
| scFv | | APGQGLEWMGRIDPYDSETHYNQKFKDRVTMTVDKSTSTAYMEL |
| | | SSLRSEDTAVYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIK |
| hzCAR123-5 | 560 | DVQLTQSPSFLSASVGDRVTITCRASKSISKDLAWYQQK |
| scFv | | PGKAPKLLIYSGSTLQSGVPSRFSGSGSGTEFTLTISSLQPEDFATYY |
| | | CQQHNKYPYTFG |
| | | GGTKVEIKGGGGSGGGGSGGGGSGGGGSQVQLVQSGAEVKKPGA |
| | | SVKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSS |
| hzCAR123-6 | 561 | EVVLTQSPATLSLSPGERATLSCRASKSISKDLAWYQQK |
| scFv | | PGQAPRLLIYSGSTLQSGIPARFSGSGSGTDFTLTISSLEPEDFAVYY |
| | | CQQHNKYPYTFG |
| | | GGTKVEIKGGGGSGGGGSGGGGSGGGGSQVQLVQSGAEVKKPGA |
| | | SVKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSS |
| hzCAR123-7 | 562 | DVVMTQSPAFLSVTPGEKVTITCRASKSISKDLAWYQQK |
| scFv | | PDQAPKLLIYSGSTLQSGVPSRFSGSGSGTDFTFTISSLEAEDAATYY |
| | | CQQHNKYPYTFG |
| | | GGTKVEIKGGGGSGGGGSGGGGSGGGGSQVQLVQSGAEVKKPGA |
| | | SVKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSS |
| hzCAR123-8 | 563 | DVVMTQSPDSLAVSLGERATINCRASKSISKDLAWYQQK |
| scFv | | PGQPPKLLIYSGSTLQSGVPDRFSGSGSGTDFTLTISSLQAEDVAVY |
| | | YCQQHNKYPYTFG |
| | | GGTKVEIKGGGGSGGGGSGGGGSGGGGSQVQLVQSGAEVKKPGA |
| | | SVKVSCKASGYTFTSY |
| | | WMNWVRQAPGQGLEWMGRIDPYDSETHYNQKFKDRVTMTVDKS |
| | | TSTAYMELSSLRSEDTA |
| | | VYYCARGNWDDYWGQGTTVTVSS |
| hzCAR123-9 | 564 | QVQLVQSGSELKKPGASVKVSCKASGYTFTSYWMNWVRQ |
| scFv | | APGQGLEWMGRIDPYDSETHYNQKFKDRFVFSVDKSVSTAYLQIS |
| | | SLKAEDTAVYYCARG |
| | | NWDDYWGQGTTVTVSSGGGGSGGGGSGGGGSGGGGSDVQLTQS |
| | | PSFLSASVGDRVTITCR |
| | | ASKSISKDLAWYQQKPGKAPKLLIYSGSTLQSGVPSRFSGSGSGTEF |
| | | TLTISSLQPEDFA |
| | | TYYCQQHNKYPYTFGGGTKVEIK |
| hzCAR123- | 565 | QVQLVQSGSELKKPGASVKVSCKASGYTFTSYWMNWVRQ |
| 10 | | APGQGLEWMGRIDPYDSETHYNQKFKDRFVFSVDKSVSTAYLQIS |
| scFv | | SLKAEDTAVYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDF |
| | | TLTISSLEPEDFA |
| | | VYYCQQHNKYPYTFGGGTKVEIK |
| hzCAR123- | 566 | QVQLVQSGSELKKPGASVKVSCKASGYTFTSYWMNWVRQ |
| 11 | | APGQGLEWMGRIDPYDSETHYNQKFKDRFVFSVDKSVSTAYLQIS |
| scFv | | SLKAEDTAVYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIK |
| hzCAR123- | 567 | QVQLVQSGSELKKPGASVKVSCKASGYTFTSYWMNWVRQ |
| 12 | | APGQGLEWMGRIDPYDSETHYNQKFKDRFVFSVDKSVSTAYLQIS |
| scFv | | SLKAEDTAVYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIK |
| hzCAR123- | 568 | DVQLTQSPSFLSASVGDRVTITCRASKSISKDLAWYQQK |
| 13 | | PGKAPKLLIYSGSTLQSGVPSRFSGSGSGTEFTLTISSLQPEDFATYY |
| scFv | | CQQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKS |
| | | VSTAYLQISSLKAEDTA |
| | | VYYCARGNWDDYWGQGTTVTVSS |
| hzCAR123- | 569 | EVVLTQSPATLSLSPGERATLSCRASKSISKDLAWYQQK |
| 14 | | PGQAPRLLIYSGSTLQSGIPARFSGSGSGTDFTLTISSLEPEDFAVYY |
| scFv | | CQQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKS |
| | | VSTAYLQISSLKAEDTA |
| | | VYYCARGNWDDYWGQGTTVTVSS |
| hzCAR123- | 570 | DVVMTQSPAFLSVTPGEKVTITCRASKSISKDLAWYQQK |
| 15 | | PDQAPKLLIYSGSTLQSGVPSRFSGSGSGTDFTFTISSLEAEDAATYY |
| scFv | | CQQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKS |
| | | VSTAYLQISSLKAEDTA |
| | | VYYCARGNWDDYWGQGTTVTVSS |
| hzCAR123- | 571 | DVVMTQSPDSLAVSLGERATINCRASKSISKDLAWYQQK |
| 16 | | PGQPPKLLIYSGSTLQSGVPDRFSGSGSGTDFTLTISSLQAEDVAVY |
| scFv | | YCQQHNKYPYTFG |
| | | GGTKVEIKGGGGSGGGGSGGGGSGGGGSQVQLVQSGSELKKPGAS |
| | | VKVSCKASGYTFTSY |
| | | WMNWVRQAPGQGLEWMGRIDPYDSETHYNQKFKDRFVFSVDKS |
| | | VSTAYLQISSLKAEDTA |
| | | VYYCARGNWDDYWGQGTTVTVSS |
| hzCAR123- | 572 | EVQLVQSGAEVKKPGESLRISCKGSGYTFTSYWMNWVRQ |
| 17 | | MPGKGLEWMGRIDPYDSETHYNQKFKDHVTISVDKSISTAYLQWS |
| scFv | | SLKASDTAMYYCARG |
| | | NWDDYWGQGTTVTVSSGGGGSGGGGSGGGGSGGGGSDVQLTQS |
| | | PSFLSASVGDRVTITCR |
| | | ASKSISKDLAWYQQKPGKAPKLLIYSGSTLQSGVPSRFSGSGSGTEF |
| | | TLTISSLQPEDFA |
| | | TYYCQQHNKYPYTFGGGTKVEIK |
| hzCAR123- | 573 | EVQLVQSGAEVKKPGESLRISCKGSGYTFTSYWMNWVRQ |
| 18 | | MPGKGLEWMGRIDPYDSETHYNQKFKDHVTISVDKSISTAYLQWS |
| scFv | | SLKASDTAMYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDF |
| | | TLTISSLEPEDFA |
| | | VYYCQQHNKYPYTFGGGTKVEIK |
| hzCAR123- | 574 | EVQLVQSGAEVKKPGESLRISCKGSGYTFTSYWMNWVRQ |
| 19 | | MPGKGLEWMGRIDPYDSETHYNQKFKDHVTISVDKSISTAYLQWS |
| scFv | | SLKASDTAMYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIK |
| hzCAR123- | 575 | EVQLVQSGAEVKKPGESLRISCKGSGYTFTSYWMNWVRQ |
| 20 | | MPGKGLEWMGRIDPYDSETHYNQKFKDHVTISVDKSISTAYLQWS |
| scFv | | SLKASDTAMYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIK |
| hzCAR123- | 576 | DVQLTQSPSFLSASVGDRVTITCRASKSISKDLAWYQQK |
| 21 | | PGKAPKLLIYSGSTLQSGVPSRFSGSGSGTEFTLTISSLQPEDFATYY |
| scFv | | CQQHNKYPYTFG |
| | | GGTKVEIKGGGGSGGGGSGGGGSGGGGSEVQLVQSGAEVKKPGE |
| | | SLRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSS |
| hzCAR123- | 577 | EVVLTQSPATLSLSPGERATLSCRASKSISKDLAWYQQK |
| 22 | | PGQAPRLLIYSGSTLQSGIPARFSGSGSGTDFTLTISSLEPEDFAVYY |
| scFv | | CQQHNKYPYTFG |
| | | GGTKVEIKGGGGSGGGGSGGGGSGGGGSEVQLVQSGAEVKKPGE |
| | | SLRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSS |
| hzCAR123- | 578 | DVVMTQSPAFLSVTPGEKVTITCRASKSISKDLAWYQQK |
| 23 | | PDQAPKLLIYSGSTLQSGVPSRFSGSGSGTDFTFTISSLEAEDAATYY |
| scFv | | CQQHNKYPYTFG |
| | | GGTKVEIKGGGGSGGGGSGGGGSGGGGSEVQLVQSGAEVKKPGE |
| | | SLRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSS |
| hzCAR123- | 579 | DVVMTQSPDSLAVSLGERATINCRASKSISKDLAWYQQK |
| 24 | | PGQPPKLLIYSGSTLQSGVPDRFSGSGSGTDFTLTISSLQAEDVAVY |
| scFv | | YCQQHNKYPYTFG |
| | | GGTKVEIKGGGGSGGGGSGGGGSGGGGSEVQLVQSGAEVKKPGE |
| | | SLRISCKGSGYTFTSY |
| | | WMNWVRQMPGKGLEWMGRIDPYDSETHYNQKFKDHVTISVDKSI |
| | | STAYLQWSSLKASDTA |
| | | MYYCARGNWDDYWGQGTTVTVSS |
| hzCAR123- | 580 | EVQLVESGGGLVQPGGSLRLSCAASGYTFTSYWMNWVRQ |
| 25 | | APGKGLVWVSRIDPYDSETHYNQKFKDRFTISVDKAKSTAYLQMN |
| scFv | | SLRAEDTAVYYCARG |
| | | NWDDYWGQGTTVTVSSGGGGSGGGGSGGGGSGGGGSDVQLTQS |
| | | PSFLSASVGDRVTITCR |
| | | ASKSISKDLAWYQQKPGKAPKLLIYSGSTLQSGVPSRFSGSGSGTEF |
| | | TLTISSLQPEDFA |
| | | TYYCQQHNKYPYTFGGGTKVEIK |
| hzCAR123- | 581 | EVQLVESGGGLVQPGGSLRLSCAASGYTFTSYWMNWVRQ |
| 26 | | APGKGLVWVSRIDPYDSETHYNQKFKDRFTISVDKAKSTAYLQMN |
| scFv | | SLRAEDTAVYYCARG |
| | | NWDDYWGQGTTVTVSSGGGGSGGGGSGGGGSGGGGSEVVLTQSP |
| | | ATLSLSPGERATLSCR |
| | | ASKSISKDLAWYQQKPGQAPRLLIYSGSTLQSGIPARFSGSGSGTDF |
| | | TLTISSLEPEDFA |
| | | VYYCQQHNKYPYTFGGGTKVEIK |
| hzCAR123- | 582 | EVQLVESGGGLVQPGGSLRLSCAASGYTFTSYWMNWVRQ |
| 27 | | APGKGLVWVSRIDPYDSETHYNQKFKDRFTISVDKAKSTAYLQMN |
| scFv | | SLRAEDTAVYYCARG |
| | | NWDDYWGQGTTVTVSSGGGGSGGGGSGGGGSGGGGSDVVMTQS |
| | | PAFLSVTPGEKVTITCR |
| | | ASKSISKDLAWYQQKPDQAPKLLIYSGSTLQSGVPSRFSGSGSGTDF |
| | | TFTISSLEAEDAA |
| | | TYYCQQHNKYPYTFGGGTKVEIK |
| hzCAR123- | 583 | EVQLVESGGGLVQPGGSLRLSCAASGYTFTSYWMNWVRQ |
| 28 | | APGKGLVWVSRIDPYDSETHYNQKFKDRFTISVDKAKSTAYLQMN |
| scFv | | SLRAEDTAVYYCARG |
| | | NWDDYWGQGTTVTVSSGGGGSGGGGSGGGGSGGGGSDVVMTQS |
| | | PDSLAVSLGERATINCR |
| | | ASKSISKDLAWYQQKPGQPPKLLIYSGSTLQSGVPDRFSGSGSGTDF |
| | | TLTISSLQAEDVA |
| | | VYYCQQHNKYPYTFGGGTKVEIK |
| hzCAR123- | 584 | DVQLTQSPSFLSASVGDRVTITCRASKSISKDLAWYQQK |
| 29 | | PGKAPKLLIYSGSTLQSGVPSRFSGSGSGTEFTLTISSLQPEDFATYY |
| scFv | | CQQHNKYPYTFG |
| | | GGTKVEIKGGGGSGGGGSGGGGSGGGGSEVQLVESGGGLVQPGG |
| | | SLRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSS |
| hzCAR123- | 585 | EVVLTQSPATLSLSPGERATLSCRASKSISKDLAWYQQK |
| 30 | | PGQAPRLLIYSGSTLQSGIPARFSGSGSGTDFTLTISSLEPEDFAVYY |
| scFv | | CQQHNKYPYTFG |
| | | GGTKVEIKGGGGSGGGGSGGGGSGGGGSEVQLVESGGGLVQPGG |
| | | SLRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSS |
| hzCAR123- | 586 | DVVMTQSPAFLSVTPGEKVTITCRASKSISKDLAWYQQK |
| 31 | | PDQAPKLLIYSGSTLQSGVPSRFSGSGSGTDFTFTISSLEAEDAATYY |
| scFv | | CQQHNKYPYTFG |
| | | GGTKVEIKGGGGSGGGGSGGGGSGGGGSEVQLVESGGGLVQPGG |
| | | SLRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSS |
| hzCAR123- | 587 | DVVMTQSPDSLAVSLGERATINCRASKSISKDLAWYQQK |
| 32 | | PGQPPKLLIYSGSTLQSGVPDRFSGSGSGTDFTLTISSLQAEDVAVY |
| scFv | | YCQQHNKYPYTFG |
| | | GGTKVEIKGGGGSGGGGSGGGGSGGGGSEVQLVESGGGLVQPGG |
| | | SLRLSCAASGYTFTSY |
| | | WMNWVRQAPGKGLVWVSRIDPYDSETHYNQKFKDRFTISVDKAK |
| | | STAYLQMNSLRAEDTA |
| | | VYYCARGNWDDYWGQGTTVTVSS |

### CD19 Antigen Binding Domain

In one embodiment, the CD19 binding domain comprises one or more (e.g., all three) light chain complementary determining region 1 (LC CDR1), light chain complementary determining region 2 (LC CDR2), and light chain complementary determining region 3 (LC CDR3) of a CD19 binding domain selected from SEQ ID NOS: 710-721, 734-745, 771, 774, 775, 777, or 780 and one or more (e.g., all three) heavy chain complementary determining region 1 (HC CDR1), heavy chain complementary determining region 2 (HC CDR2), and heavy chain complementary determining region 3 (HC CDR3) of a CD19 binding domain selected from SEQ ID NOS: 710-721, 734-745, 771, 774, 775, 777, or 780. In one embodiment, the CD19 binding domain comprises a light chain variable region described herein (e.g., in Table 13A or 14A) and/or a heavy chain variable region described herein (e.g., in Table 13A or 14A). In one embodiment, the CD19 binding domain is a scFv comprising a light chain variable region and a heavy chain variable region of an amino acid sequence of Table 13A or 14A. In an embodiment, the CD19 binding domain (e.g., an scFV) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 13A or 14A, or a sequence with at least 95% (e.g., 95-99%) identity to an amino acid sequence of Table 13A or 14A; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 13A or 14A, or a sequence with 95% (e.g., 95-99%) identity to an amino acid sequence of Table 13A or 14A.

In one embodiment, the CD19 binding domain comprises a light chain variable region comprising an amino acid sequence described herein, e.g., in Table 13A or 14A, is attached to a heavy chain variable region comprising an amino acid sequence described herein, e.g., in Table 13A or 14A, via a linker, e.g., a linker described herein. In one embodiment, the humanized anti-CD19 binding domain includes a (Gly4-Ser)n linker (SEQ ID NO: 26), wherein n is 1, 2, 3, 4, 5, or 6, preferably 3 or 4. The light chain variable region and heavy chain variable region of a scFv can be, e.g., in any of the following orientations: light chain variable region-linker-heavy chain variable region or heavy chain variable region-linker-light chain variable region.

In another embodiment, the CD19 binding domain comprises any antibody or antibody fragment thereof known in the art that binds to CD19.

In one embodiment, the framework region can comprise, one, two, three, four or five modifications, e.g., substitutions, e.g., from the amino acid at the corresponding murine sequence (e.g., of SEQ ID NO: 774). In one embodiment, the framework region, e.g., all four framework regions of the light chain variable region are derived from a VK3 1.25 germline sequence. In one embodiment, the framework region can comprise, one, two, three, four or five modifications, e.g., substitutions, e.g., from the amino acid at the corresponding murine sequence (e.g., of SEQ ID NO: 774).

### Exemplary CD19 Antigen Binding Domains and CAR Constructs

Exemplary CD19 CAR constructs disclosed herein comprise a scFv (e.g., a human scFv) as disclosed in Table 13A or 14A herein, optionally preceded with an optional leader sequence (e.g., SEQ ID NO:1 and SEQ ID NO:12 for exemplary leader amino acid and nucleotide sequences, respectively). The sequences of the scFv fragments (amino acid sequences of SEQ ID NOs: 710-721, 734-745, 771, 774, 775, 777, or 780) are provided herein in Table 13A or 14A. The CD19 CAR construct can further include an optional hinge domain, e.g., a CD8 hinge domain (e.g., including the amino acid sequence of SEQ ID NO: 2 or encoded by a nucleic acid sequence of SEQ ID NO:13); a transmembrane domain, e.g., a CD8 transmembrane domain (e.g., including the amino acid sequence of SEQ ID NO: 6 or encoded by the nucleotide sequence of SEQ ID NO: 17); an intracellular domain, e.g., a 4-1BB intracellular domain (e.g., including the amino acid sequence of SEQ ID NO: 7 or encoded by the nucleotide sequence of SEQ ID NO: 18; and a functional signaling domain, e.g., a CD3 zeta domain (e.g., including amino acid sequence of SEQ ID NO: 9 or 10, or encoded by the nucleotide sequence of SEQ ID NO: 20 or 21). In certain embodiments, the domains are contiguous with and in the same reading frame to form a single fusion protein. In other embodiments, the domain are in separate polypeptides, e.g., as in an RCAR molecule as described herein.

In certain embodiments, the full length CD19 CAR molecule includes the amino acid sequence of, or is encoded by the nucleotide sequence of, CAR1-CAR12, CTL019, mCAR1-mCAR3, or SSJ25-C1, provided in Table 13A or 14A, or a sequence substantially identical (e.g., at least 95%, e.g., 95-99% identical thereto, or up to 20, 15, 10, 8, 6, 5, 4, 3, 2, or 1 amino acid changes) to any of the aforesaid sequences.

In certain embodiments, the CD19 CAR molecule, or the CD19 antigen binding domain, includes the scFv amino acid sequence of, or is encoded by the nucleotide sequence of, CAR1-CAR12, CTL019, mCAR1-mCAR3, or SSJ25-C1, provided in Table 13A or 14A, or a sequence substantially identical (e.g., at least 95%, e.g., 95-99% identical thereto, or up to 20, 15, 10, 8, 6, 5, 4, 3, 2, or 1 amino acid changes) to any of the aforesaid sequences.

In certain embodiments, the CD19 CAR molecule, or the CD19 antigen binding domain, includes the heavy chain variable region and/or the light chain variable region of CAR1-CAR12, CTL019, mCAR1-mCAR3, or SSJ25-C1, provided in Table 13A or 14A, or a sequence substantially identical (e.g., at least 95%, e.g., 95-99% identical, or up to 20, 15, 10, 8, 6, 5, 4, 3, 2, or 1 amino acid changes) to any of the aforesaid sequences.

In certain embodiments, the CD19 CAR molecule, or the CD19 antigen binding domain, includes one, two or three CDRs from the heavy chain variable region (e.g., HCDR1, HCDR2 and/or HCDR3) of CAR1-CAR12, CTL019, mCAR1-mCAR3, or SSJ25-C1, provided in Table 13A or 14A; and/or one, two or three CDRs from the light chain variable region (e.g., LCDR1, LCDR2 and/or LCDR3) of CAR1-CAR12, CTL019, mCAR1-mCAR3, or SSJ25-C1, provided in Table 13A or 14A; or a sequence substantially identical (e.g., at least 95%, e.g., 95-99% identical, or up to 5, 4, 3, 2, or 1 amino acid changes) to any of the aforesaid sequences.

The sequences of CDR sequences of the scFv domains are shown in Table 15A for the heavy chain variable domains and in Table 16A for the light chain variable domains.

The amino acid and nucleic acid sequences of the CD19 scFv domains and CD19 CAR molecules are provided in Tables 13A and 14A. In one embodiment, the CD19 CAR molecule includes a leader sequence described herein, e.g., as underlined in the sequences provided in Tables 13A and 14A. In one embodiment, the CD19 CAR molecule does not include a leader sequence.

In embodiments, the CAR molecule comprises an antigen binding domain that binds specifically to CD19 (CD19 CAR). In one embodiment, the antigen binding domain targets human CD19. In one embodiment, the antigen binding domain of the CAR has the same or a similar binding specificity as the FMC63 scFv fragment described in Nicholson et al. Mol. Immun. 34 (16-17): 1157-1165 (1997). In one embodiment, the antigen binding domain of the CAR includes the scFv fragment described in Nicholson et al. Mol. Immun. 34 (16-17): 1157-1165 (1997). A CD19 antibody molecule can be, e.g., an antibody molecule (e.g., a humanized anti-CD19 antibody molecule) described in WO2014/153270, which is incorporated herein by reference in its entirety. WO2014/153270 also describes methods of assaying the binding and efficacy of various CAR constructs.

In one aspect, the parental murine scFv sequence is the CAR19 construct provided in PCT publication WO2012/079000 (incorporated herein by reference) and provided herein as SEQ ID NO: 773. In one embodiment, the anti-CD19 binding domain is a scFv described in WO2012/079000 and provided herein in SEQ ID NO: 774.

In one embodiment, the CAR molecule comprises the polypeptide sequence provided as SEQ ID NO: 12 in PCT publication WO2012/079000, and provided herein as SEQ ID NO: 773, wherein the scFv domain is substituted by one or more sequences selected from SEQ ID NOS: 758-769. In one embodiment, the scFv domains of SEQ ID NOS: 758-769 are humanized variants of the scFv domain of SEQ ID NO: 774 which is an scFv fragment of murine origin that specifically binds to human CD19. Humanization of this mouse scFv may be desired for the clinical setting, where the mouse-specific residues may induce a human-anti-mouse antigen (HAMA) response in patients who receive CART19 treatment, e.g., treatment with T cells transduced with the CAR19 construct.

In one embodiment, the CD19 CAR comprises an amino acid sequence provided as SEQ ID NO: 12 in PCT publication WO2012/079000. In embodiment, the amino acid sequence is

(SEQ ID NO: 773)

MALPVTALLLPLALLLHAARPdiqmtqttsslsaslgdrvtiscrasqdi
skylnwyqqkpdgtvklliyhtsrlhsgvpsrfsgsgsgtdysltisnle
qediatyfcqqgntlpytfgggtkleitggggsggggsggggsevklqes
gpglvapsqslsvtctvsgvslpdygvswirqpprkglewlgviwgsett
yynsalksrltiikdnsksqvflkmnslqtddtaiyycakhyyyggsyam
dywgqgtsvtvsstttpaprpptpaptiasqplslrpeacrpaaggavht
rgldfacdiyiwaplagtcgvlllslvitlyckrgrkkllyifkqpfmrp
vqttqeedgcscrfpeeeeggcelrvkfsrsadapaykqgqnqlynelnl
grreeydvldkrrgrdpemggkprrknpqeglynelqkdkmaeayseigm
kgerrrgkghdglyqglstatkdtydalhmqalppr, or asequence
substantially homologous thereto.

In embodiment, the amino acid sequence is:

(SEQ ID NO: 793)

diqmtqttsslsaslgdrvtiscrasqdiskylnwyqqkpdgtvklli
yhtsrlhsgvpsrfsgsgsgtdysltisnleqediat
yfcqqgntlpytfgggtkleitggggsggggsggggsevklqesg
pglvapsqslsvtctvsgvslpdygvswirqpprkglewlgv
iwgsettyynsalksrltiikdnsksqvflkmnslqtddtaiyycakh
yyyggsyamdywgqgtsvtvsstttpaprpptpaptiasq
plslrpeacrpaaggavhtrgldfacdiyiwaplagtcgvlllslvit
lyckrgrkkllyifkqpfmrpvqttqeedgcscrfpeeeeggc
elrvkfsrsadapaykqgqnqlynelnlgrreeydvldkrrgrdpe
mggkprrknpqeglynelqkdkmaeayseigmkgerrrg
kghdglyqglstatkdtydalhmqalppr, or a sequence
substantially homologous thereto.

In one embodiment, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CD19 CAR comprises an antigen binding domain (e.g., a humanized antigen binding domain) according to Table 3 of WO2014/153270, incorporated herein by reference.

In embodiments, the CAR molecule is a CD19 CAR molecule described herein, e.g., a humanized CAR molecule described herein, e.g., a humanized CD19 CAR molecule of Table 13A or having CDRs as set out in Tables 15A and 16A.

In embodiments, the CAR molecule is a CD19 CAR molecule described herein, e.g., a murine CAR molecule described herein, e.g., a murine CD19 CAR molecule of Table 14A or having CDRs as set out in Tables 15A and 16A.

In some embodiments, the CAR molecule comprises one, two, and/or three CDRs from the heavy chain variable region and/or one, two, and/or three CDRs from the light chain variable region of the murine or humanized CD19 CAR of Table 15A and 16A.

In one embodiment, the antigen binding domain comprises one, two three (e.g., all three) heavy chain CDRs, HC CDR1, HC CDR2 and HC CDR3, from an antibody listed herein, and/or one, two, three (e.g., all three) light chain CDRs, LC CDR1, LC CDR2 and LC CDR3, from an antibody listed herein. In one embodiment, the antigen binding domain comprises a heavy chain variable region and/or a variable light chain region of an antibody listed herein.

### Humanization of Murine Anti-CD19 Antibody

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159), for instance Tables 3, 4, and 5 (p. 125-147).

### CAR Constructs, e.g., CD19 CAR Constructs

Of the CD19 CAR constructs described in International Application WO2014/153270, certain sequences are reproduced herein.

The sequences of the humanized scFv fragments (SEQ ID NOS: 710-721) are provided below in Table 13A. Full CAR constructs were generated using SEQ ID NOs: 710-721with additional sequences, e.g., from the “CAR constructs components” section herein, to generate full CAR constructs with SEQ ID NOs: 758-769.

These clones all contained a Q/K residue change in the signal domain of the co-stimulatory domain derived from 4-1BB.

TABLE 13A
Humanized CD19 CAR Constructs

| | | |
|---|---|---|
| | SEQ | |
| Name | ID | Sequence |

CAR 1

| | | |
|---|---|---|
| CAR1 | 710 | EIVMTQSPATLSLSPGERATLSCRASQDISKYLNWYQQKPGQA |
| scFv | | PRLLIYHTSRLHSGIPARFSGSGSGTDYTLTISSLQPEDFAVYFC |
| domain | | QQGNTLPYTFGQGTKLEIKGGGGSGGGGSGGGGSQVQLQESG |
| | | PGLVKPSETLSLTCTVSGVSLPDYGVSWIRQPPGKGLEWIGVI |
| | | WGSETTYYSSSLKSRVTISKDNSKNQVSLKLSSVTAADTAVYY |
| | | CAKHYYYGGSYAMDYWGQGTLVTVSS |
| 103101 | 722 | atggccctccctgtcaccgccctgctgcttccgctggctcttctgctccacgccgctcggcccgaaa |
| CAR1 | | ttgtgatgacccagtcacccgccactcttagcctttcacccggtgagcgcgcaaccctgtcttgcag |
| Soluble | | agcctcccaagacatctcaaaataccttaattggtatcaacagaagcccggacaggctcctcgcctt |
| scFv-nt | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactactcttcatccctcaagtcacgcgtcaccatctcaaaggacaactctaagaatcaggtgtcac |
| | | tgaaactgtcatctgtgaccgcagccgacaccgccgtgtactattgcgctaagcattactattatggc |
| | | gggagctacgcaatggattactggggacagggtactctggtcaccgtgtccagccaccaccatcat |
| | | caccatcaccat |
| 103101 | 734 | MALPVTALLLPLALLLHAARPeivmtqspatlslspgeratlscrasqdiskyl |
| CAR1 | | nwyqqkpgqaprlliyhtsrlhsgiparfsgsgsgtdytltisslqpedfavyfcqqgntlpytfgq |
| Soluble | | gtkleikggggsggggsggggsqvqlqesgpglvkpsetlsltctvsgvslpdygvswirqppg |
| scFv-aa | | kglewigviwgsettyyssslksrvtiskdnsknqvslklssvtaadtavyycakhyyyggsya |
| | | mdywgqgtlvtvsshhhhhhhh |
| 104875 | 746 | atggccctccctgtcaccgccctgctgcttccgctggctcttctgctccacgccgctcggcccgaaa |
| CAR1- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactactcttcatccctcaagtcacgcgtcaccatctcaaaggacaactctaagaatcaggtgtcac |
| | | tgaaactgtcatctgtgaccgcagccgacaccgccgtgtactattgcgctaagcattactattatggc |
| | | gggagctacgcaatggattactggggacagggtactctggtcaccgtgtccagcaccactacccc |
| | | agcaccgaggccacccaccccggctcctaccatcgcctcccagcctctgtccctgcgtccggagg |
| | | catgtagacccgcagctggtggggccgtgcatacccggggtcttgacttcgcctgcgatatctacat |
| | | ttgggcccctctggctggtacttgcggggtcctgctgctttcactcgtgatcactctttactgtaagcgc |
| | | ggtcggaagaagctgctgtacatctttaagcaacccttcatgaggcctgtgcagactactcaagagg |
| | | aggacggctgttcatgccggttcccagaggaggaggaaggcggctgcgaactgcgcgtgaaatt |
| | | cagccgcagcgcagatgctccagcctacaagcaggggcagaaccagctctacaacgaactcaat |
| | | cttggtcggagagaggagtacgacgtgctggacaagcggagaggacgggacccagaaatgggc |
| | | gggaagccgcgcagaaagaatccccaagagggcctgtacaacgagctccaaaaggataagatg |
| | | gcagaagcctatagcgagattggtatgaaaggggaacgcagaagaggcaaaggccacgacgga |
| | | ctgtaccagggactcagcaccgccaccaaggacacctatgacgctcttcacatgcaggccctgcc |
| | | gcctcgg |
| 104875 | 758 | MALPVTALLLPLALLLHAARPeivmtqspatlslspgeratlscrasadiskyln |
| CAR 1- | | wyqqkpgqaprlliyhtsrlhsgiparfsgsgsgtdytltisslqpedfavyfcqqgntlpytfgq |
| Full-aa | | gtkleikggggsggggsggggsqvqlqesgpglvkpsetlsltctvsgvslpdygvswirqppg |
| | | kglewigviwgsettyyssslksrvtiskdnsknqvslklssvtaadtavyycakhyyyggsya |
| | | mdywgqgtlvtvsstttpaprpptpaptiasqplslrpeacrpaaggavhtrgldfacdiyiwapl |
| | | agtcgvlllslvitlyckrgrkkllyifkqpfmrpvqttqeedgcscrfpeeeeggcelrvkfsrsad |
| | | apaykqgqnqlynelnlgrreeydvldkrrgrdpemggkprrknpqeglynelqkdkmaea |
| | | yseigmkgerrrgkghdglyqglstatkdtydalhmqalppr |

CAR 2

| | | |
|---|---|---|
| CAR2 | 711 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsqvqlqesgpg |
| domain | | lvkpsetlsltctvsgvslpdygvswirqppgkglewigviwgsettyyqsslksrvtiskdnskn |
| | | qvslklssvtaadtavyycakhyyyggsyamdywgqgtlvtvss |
| 103102 | 723 | atggccctccctgtcaccgccctgctgcttccgctggctcttctgctccacgccgctcggcccgaaa |
| CAR2- | | ttgtgatgacccagtcacccgccactcttagcctttcacccggtgagcgcgcaaccctgtcttgcag |
| Soluble | | agcctcccaagacatctcaaaataccttaattggtatcaacagaagcccggacaggctcctcgcctt |
| scFv-nt | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactaccaatcatccctcaagtcacgcgtcaccatctcaaaggacaactctaagaatcaggtgtca |
| | | ctgaaactgtcatctgtgaccgcagccgacaccgccgtgtactattgcgctaagcattactattatgg |
| | | cgggagctacgcaatggattactggggacagggtactctggtcaccgtgtccagccaccaccatc |
| | | atcaccatcaccat |
| 103102 | 735 | MALPVTALLLPLALLLHAARPeivmtqspatlslspgeratlscrasqdiskyl |
| CAR2- | | nwyqqkpgqaprlliyhtsrlhsgiparfsgsgsgtdytltisslqpedfavyfcqqgntlpytfgq |
| Soluble | | gtkleikggggsggggsggggsqvqlqesgpglvkpsetlsltctvsgvslpdygvswirqppg |
| scFv-aa | | kglewigviwgsettyyqsslksrvtiskdnsknqvslklssvtaadtavyycakhyyyggsya |
| | | mdywgqgtlvtvsshhhhhhhh |
| 104876 | 747 | atggccctccctgtcaccgccctgctgcttccgctggctcttctgctccacgccgctcggcccgaaa |
| CAR 2- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactaccaatcatccctcaagtcacgcgtcaccatctcaaaggacaactctaagaatcaggtgtca |
| | | ctgaaactgtcatctgtgaccgcagccgacaccgccgtgtactattgcgctaagcattactattatgg |
| | | cgggagctacgcaatggattactggggacagggtactctggtcaccgtgtccagcaccactaccc |
| | | cagcaccgaggccacccaccccggctcctaccatcgcctcccagcctctgtccctgcgtccggag |
| | | gcatgtagacccgcagctggtggggccgtgcatacccggggtcttgacttcgcctgcgatatctac |
| | | atttgggcccctctggctggtacttgcggggtcctgctgctttcactcgtgatcactctttactgtaagc |
| | | gcggtcggaagaagctgctgtacatctttaagcaacccttcatgaggcctgtgcagactactcaaga |
| | | ggaggacggctgttcatgccggttcccagaggaggaggaaggcggctgcgaactgcgcgtgaa |
| | | attcagccgcagcgcagatgctccagcctacaagcaggggcagaaccagctctacaacgaactc |
| | | aatcttggtcggagagaggagtacgacgtgctggacaagcggagaggacgggacccagaaatg |
| | | ggcgggaagccgcgcagaaagaatccccaagagggcctgtacaacgagctccaaaaggataag |
| | | atggcagaagcctatagcgagattggtatgaaaggggaacgcagaagaggcaaaggccacgac |
| | | ggactgtaccagggactcagcaccgccaccaaggacacctatgacgctcttcacatgcaggccct |
| | | gccgcctcgg |
| 104876 | 759 | MALPVTALLLPLALLLHAARPeivmtqspatlslspgeratlscrasadiskyln |
| CAR 2- | | wyqqkpgqaprlliyhtsrlhsgiparfsgsgsgtdytltisslqpedfavyfcqqgntlpytfgq |
| Full-aa | | gtkleikggggsggggsggggsqvqlqesgpglvkpsetlsltctvsgvslpdygvswirqppg |
| | | kglewigviwgsettyyqsslksrvtiskdnsknqvslklssvtaadtavyycakhyyyggsy |
| | | amdywgqgtlvtvsstttpaprpptpaptiasqplslrpeacrpaaggavhtrgldfacdiyiwa |
| | | plagtcgvlllslvitlyckrgrkkllyifkqpfmrpvqttqeedgcscrfpeeeeggcelrvkfsrs |
| | | adapaykqgqnqlynelnlgrreeydvldkrrgrdpemggkprrknpqeglynelqkdkma |
| | | eayseigmkgerrrgkghdglyqglstatkdtydalhmqalppr |

CAR 3

| | | |
|---|---|---|
| CAR3 | 712 | qvqlqesgpglvkpsetlsltctvsgvslpdygvswirqppgkglewigviwgsettyyssslks |
| scFv | | rvtiskdnsknqvslklssvtaadtavyycakhyyyggsyamdywgqgtlvtvssggggsgg |
| domain | | ggsggggseivmtqspatlslspgeratlscrasqdiskylnwyqqkpgqaprlliyhtsrlhsgi |
| | | parfsgsgsgtdytltisslqpedfavyfcqqgntlpytfgqgtkleik |
| 103104 | 724 | atggctctgcccgtgaccgcactcctcctgccactggctctgctgcttcacgccgctcgcccacaag |
| CAR 3- | | tccagcttcaagaatcagggcctggtctggtgaagccatctgagactctgtccctcacttgcaccgtg |
| Soluble | | agcggagtgtccctcccagactacggagtgagctggattagacagcctcccggaaagggactgg |
| scFv-nt | | agtggatcggagtgatttggggtagcgaaaccacttactattcatcttccctgaagtcacgggtcacc |
| | | atttcaaaggataactcaaagaatcaagtgagcctcaagctctcatcagtcaccgccgctgacaccg |
| | | ccgtgtattactgtgccaagcattactactatggagggtcctacgccatggactactggggccaggg |
| | | aactctggtcactgtgtcatctggtggaggaggtagcggaggaggcgggagcggtggaggtggc |
| | | tccgaaatcgtgatgacccagagccctgcaaccctgtccctttctcccggggaacgggctacccttt |
| | | cttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaacatcaccac |
| | | catcatcaccatcac |
| 103104 | 736 | MALPVTALLLPLALLLHAARPqvqlqesgpglvkpsetlsltctvsgvslpdy |
| CAR 3- | | gvswirqppgkglewigviwgsettyyssslksrvtiskdnsknqvslklssvtaadtavyycak |
| Soluble | | hyyyggsyamdywgqgtlvtvssggggsggggsggggseivmtqspatlslspgeratlscra |
| scFv-aa | | sqdiskylnwyqqkpgqaprlliyhtsrlhsgiparfsgsgsgtdytltisslqpedfavyfcqqgn |
| | | tlpytfgqgtkleikhhhhhhhh |
| 104877 | 748 | atggctctgcccgtgaccgcactcctcctgccactggctctgctgcttcacgccgctcgcccacaag |
| CAR 3- | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactattcatcttccctgaagtcacgggtcacc |
| | | atttcaaaggataactcaaagaatcaagtgagcctcaagctctcatcagtcaccgccgctgacaccg |
| | | ccgtgtattactgtgccaagcattactactatggagggtcctacgccatggactactggggccaggg |
| | | aactctggtcactgtgtcatctggtggaggaggtagcggaggaggcgggagcggtggaggtggc |
| | | tccgaaatcgtgatgacccagagccctgcaaccctgtccctttctcccggggaacgggctacccttt |
| | | cttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaaaccactact |
| | | cccgctccaaggccacccacccctgccccgaccatcgcctctcagccgctttccctgcgtccgga |
| | | ggcatgtagacccgcagctggtggggccgtgcatacccggggtcttgacttcgcctgcgatatcta |
| | | catttgggcccctctggctggtacttgcggggtcctgctgctttcactcgtgatcactctttactgtaag |
| | | cgcggtcggaagaagctgctgtacatctttaagcaacccttcatgaggcctgtgcagactactcaag |
| | | aggaggacggctgttcatgccggttcccagaggaggaggaaggcggctgcgaactgcgcgtga |
| | | aattcagccgcagcgcagatgctccagcctacaagcaggggcagaaccagctctacaacgaact |
| | | caatcttggtcggagagaggagtacgacgtgctggacaagcggagaggacgggacccagaaat |
| | | gggcgggaagccgcgcagaaagaatccccaagagggcctgtacaacgagctccaaaaggataa |
| | | gatggcagaagcctatagcgagattggtatgaaaggggaacgcagaagaggcaaaggccacga |
| | | cggactgtaccagggactcagcaccgccaccaaggacacctatgacgctcttcacatgcaggccc |
| | | tgccgcctcgg |
| 104877 | 760 | MALPVTALLLPLALLLHAARPqvqlqesgpglvkpsetlsltctvsgvslpdygv |
| CAR 3- | | swirqppgkglewigviwgsettyyssslksrvtiskdnsknqvslklssvtaadtavyycakh |
| Full-aa | | yyyggsyamdywgqgtlvtvssggggsggggsggggseivmtqspatlslspgeratlscra |
| | | sqdiskylnwyqqkpgqaprlliyhtsrlhsgiparfsgsgsgtdytitisslqpedfavyfcqq |
| | | gntlpytfgqgtkleiktttpaprpptpaptiasqplslrpeacrpaaggavhtrgldfacdiyiwa |
| | | plagtcgvlllslvitlyckrgrkkllyifkqpfmrpvqttqeedgcscrfpeeeeggcelrvkfsrs |
| | | adapaykqgqnqlynelnlgrreeydvldkrrgrdpemggkprrknpqeglynelqkdkma |
| | | eayseigmkgerrrgkghdglyqglstatkdtydalhmqalppr |

CAR 4

| | | |
|---|---|---|
| CAR4 | 713 | qvqlqesgpglvkpsetlsltctvsgvslpdygvswirqppgkglewigviwgsettyyqsslks |
| scFv | | rvtiskdnsknqvslklssvtaadtavyycakhyyyggsyamdywgqgtlvtvssggggsgg |
| domain | | ggsggggseivmtqspatlslspgeratlscrasqdiskylnwyqqkpgqaprlliyhtsrlhsgi |
| | | parfsgsgsgtdytltisslqpedfavyfcqqgntlpytfgqgtkleik |
| 103106 | 725 | atggctctgcccgtgaccgcactcctcctgccactggctctgctgcttcacgccgctcgcccacaag |
| CAR4- | | tccagcttcaagaatcagggcctggtctggtgaagccatctgagactctgtccctcacttgcaccgtg |
| Soluble | | agcggagtgtccctcccagactacggagtgagctggattagacagcctcccggaaagggactgg |
| scFv-nt | | agtggatcggagtgatttggggtagcgaaaccacttactatcaatcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccgaaatcgtgatgacccagagccctgcaaccctgtccctttctcccggggaacgggctaccctt |
| | | tcttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaacatcaccac |
| | | catcatcaccatcac |
| 103106 | 737 | MALPVTALLLPLALLLHAARPqvqlqesgpglvkpsetlsltctvsgvslpdy |
| CAR4- | | gvswirqppgkglewigviwgsettyyqsslksrvtiskdnsknqvslklssvtaadtavyyca |
| Soluble | | khyyyggsyamdywgqgtlvtvssggggsggggsggggseivmtqspatlslspgeratlscr |
| scFv-aa | | asqdiskylnwyqqkpgqaprlliyhtsrlhsgiparfsgsgsgtdytltisslqpedfavyfcqqg |
| | | ntlpytfgqgtkleikhhhhhhhh |
| 104878 | 749 | atggctctgcccgtgaccgcactcctcctgccactggctctgctgcttcacgccgctcgcccacaag |
| CAR 4- | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactatcaatcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccgaaatcgtgatgacccagagccctgcaaccctgtccctttctcccggggaacgggctaccctt |
| | | tcttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaaaccactact |
| | | cccgctccaaggccacccacccctgccccgaccatcgcctctcagccgctttccctgcgtccgga |
| | | ggcatgtagacccgcagctggtggggccgtgcatacccggggtcttgacttcgcctgcgatatcta |
| | | catttgggcccctctggctggtacttgcggggtcctgctgctttcactcgtgatcactctttactgtaag |
| | | cgcggtcggaagaagctgctgtacatctttaagcaacccttcatgaggcctgtgcagactactcaag |
| | | aggaggacggctgttcatgccggttcccagaggaggaggaaggcggctgcgaactgcgcgtga |
| | | aattcagccgcagcgcagatgctccagcctacaagcaggggcagaaccagctctacaacgaact |
| | | caatcttggtcggagagaggagtacgacgtgctggacaagcggagaggacgggacccagaaat |
| | | gggcgggaagccgcgcagaaagaatccccaagagggcctgtacaacgagctccaaaaggataa |
| | | gatggcagaagcctatagcgagattggtatgaaaggggaacgcagaagaggcaaaggccacga |
| | | cggactgtaccagggactcagcaccgccaccaaggacacctatgacgctcttcacatgcaggccc |
| | | tgccgcctcgg |
| 104878 | 761 | MALPVTALLLPLALLLHAARPqvqlqesgpglvkpsetlsltctvsgvslpdygv |
| CAR 4- | | swirqppgkglewigviwgsettyyqsslksrvtiskdnsknqvslklssvtaadtavyycakh |
| Full-aa | | yyyggsyamdywgqgtlvtvssggggsggggsggggseivmtqspatlslspgeratlscra |
| | | sqdiskylnwyqqkpgqaprlliyhtsrlhsgiparfsgsgsgtdytltisslqpedfavyfcqq |
| | | gntlpytfgqgtkleiktttpaprpptpaptiasqplslrpeacrpaaggavhtrgldfacdiyiwa |
| | | plagtcgvlllslvitlyckrgrkkllyifkqpfmrpvqttqeedgcscrfpeeeeggcelrvkfsrs |
| | | adapaykqgqnqlynelnlgrreeydvldkrrgrdpemggkprrknpqeglynelqkdkma |
| | | eayseigmkgerrrgkghdglyqglstatkdtydalhmqalppr |

CAR 5

| | | |
|---|---|---|
| CAR 5 | 714 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsggggsqvqlq |
| domain | | esgpglvkpsetlsltctvsgvslpdygvswirqppgkglewigviwgsettyyssslksrvtisk |
| | | dnsknqvslklssvtaadtavyycakhyyyggsyamdywgqgtlvtvss |
| 99789 | 726 | atggccctcccagtgaccgctctgctgctgcctctcgcacttcttctccatgccgctcggcctgagat |
| CAR5- | | cgtcatgacccaaagccccgctaccctgtccctgtcacccggcgagagggcaaccctttcatgcag |
| Soluble | | ggccagccaggacatttctaagtacctcaactggtatcagcagaagccagggcaggctcctcgcct |
| scFv-nt | | gctgatctaccacaccagccgcctccacagcggtatccccgccagattttccgggagcgggtctgg |
| | | aaccgactacaccctcaccatctcttctctgcagcccgaggatttcgccgtctatttctgccagcagg |
| | | ggaatactctgccgtacaccttcggtcaaggtaccaagctggaaatcaagggaggcggaggatca |
| | | ggcggtggcggaagcggaggaggtggctccggaggaggaggttcccaagtgcagcttcaagaa |
| | | tcaggacccggacttgtgaagccatcagaaaccctctccctgacttgtaccgtgtccggtgtgagcc |
| | | tccccgactacggagtctcttggattcgccagcctccggggaagggtcttgaatggattggggtgat |
| | | ttggggatcagagactacttactactcttcatcacttaagtcacgggtcaccatcagcaaagataata |
| | | gcaagaaccaagtgtcacttaagctgtcatctgtgaccgccgctgacaccgccgtgtactattgtgc |
| | | caaacattactattacggagggtcttatgctatggactactggggacaggggaccctggtgactgtct |
| | | ctagccatcaccatcaccaccatcatcac |
| 99789 | 738 | MALPVTALLLPLALLLHAARPeivmtqspatlslspgeratlscrasqdiskyl |
| CAR5- | | nwyqqkpgqaprlliyhtsrlhsgiparfsgsgsgtdytltisslqpedfavyfcqqgntlpytfgq |
| Soluble | | gtkleikggggsggggsggggsggggsqvqlqesgpglvkpsetlsltctvsgvslpdygvswi |
| scFv-aa | | rqppgkglewigviwgsettyyssslksrvtiskdnsknqvslklssvtaadtavyycakhyyy |
| | | ggsyamdywgqgtlvtvsshhhhhhhh |
| 104879 | 750 | atggccctccctgtcaccgccctgctgcttccgctggctcttctgctccacgccgctcggcccgaaa |
| CAR 5- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagcggcggaggcgggagccaggtccaactccaaga |
| | | aagcggaccgggtcttgtgaagccatcagaaactctttcactgacttgtactgtgagcggagtgtctc |
| | | tccccgattacggggtgtcttggatcagacagccaccggggaagggtctggaatggattggagtg |
| | | atttggggctctgagactacttactactcttcatccctcaagtcacgcgtcaccatctcaaaggacaac |
| | | tctaagaatcaggtgtcactgaaactgtcatctgtgaccgcagccgacaccgccgtgtactattgcg |
| | | ctaagcattactattatggcgggagctacgcaatggattactggggacagggtactctggtcaccgt |
| | | gtccagcaccactaccccagcaccgaggccacccaccccggctcctaccatcgcctcccagcctc |
| | | tgtccctgcgtccggaggcatgtagacccgcagctggtggggccgtgcatacccggggtcttgact |
| | | tcgcctgcgatatctacatttgggcccctctggctggtacttgcggggtcctgctgctttcactcgtgat |
| | | cactctttactgtaagcgcggtcggaagaagctgctgtacatctttaagcaacccttcatgaggcctg |
| | | tgcagactactcaagaggaggacggctgttcatgccggttcccagaggaggaggaaggcggctg |
| | | cgaactgcgcgtgaaattcagccgcagcgcagatgctccagcctacaagcaggggcagaaccag |
| | | ctctacaacgaactcaatcttggtcggagagaggagtacgacgtgctggacaagcggagaggac |
| | | gggacccagaaatgggcgggaagccgcgcagaaagaatccccaagagggcctgtacaacgag |
| | | ctccaaaaggataagatggcagaagcctatagcgagattggtatgaaaggggaacgcagaagag |
| | | gcaaaggccacgacggactgtaccagggactcagcaccgccaccaaggacacctatgacgctct |
| | | tcacatgcaggccctgccgcctcgg |
| 104879 | 762 | MALPVTALLLPLALLLHAARPeivmtqspatlslspgeratlscrasqdiskyln |
| CAR 5- | | wyqqkpgqaprlliyhtsrlhsgiparfsgsgsgtdytltisslqpedfavyfcqqgntlpytfgq |
| Full-aa | | gtkleikggggsggggsggggsggggsqvqlqesgpglykpsetlsltctvsgyslpdygvswi |
| | | rqppgkglewigviwgsettyyssslksrvtiskdnsknqvslklssytaadtavyycakhyyy |
| | | ggsyamdywgqgtlvtvsstttpaprpptpaptiasqplslrpeacrpaaggavhtrgldfacdi |
| | | yiwaplagtcgvlllslvitlyckrgrkkllyifkqpfmrpvqttqeedgcscrfpeeeeggcelrv |
| | | kfsrsadapaykqgqnqlynelnlgrreeydvldkrrgrdpemggkprrknpqeglynelqkd |
| | | kmaeayseigmkgerrrgkghdglyqglstatkdtydalhmqalppr |

CAR 6

| | | |
|---|---|---|
| CAR6 | 715 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsggggsqvqlq |
| domain | | esgpglvkpsetlsltctvsgvslpdygvswirqppgkglewigviwgsettyyqsslksrvtisk |
| | | dnsknqvslklssvtaadtavyycakhyyyggsyamdywgqgtlvtvss |
| 99790 | 727 | atggccctcccagtgaccgctctgctgctgcctctcgcacttcttctccatgccgctcggcctgagat |
| CAR6- | | cgtcatgacccaaagccccgctaccctgtccctgtcacccggcgagagggcaaccctttcatgcag |
| Soluble | | ggccagccaggacatttctaagtacctcaactggtatcagcagaagccagggcaggctcctcgcct |
| scFv-nt | | gctgatctaccacaccagccgcctccacagcggtatccccgccagattttccgggagcgggtctgg |
| | | aaccgactacaccctcaccatctcttctctgcagcccgaggatttcgccgtctatttctgccagcagg |
| | | ggaatactctgccgtacaccttcggtcaaggtaccaagctggaaatcaagggaggcggaggatca |
| | | ggcggtggcggaagcggaggaggtggctccggaggaggaggttcccaagtgcagcttcaagaa |
| | | tcaggacccggacttgtgaagccatcagaaaccctctccctgacttgtaccgtgtccggtgtgagcc |
| | | tccccgactacggagtctcttggattcgccagcctccggggaagggtcttgaatggattggggtgat |
| | | ttggggatcagagactacttactaccagtcatcacttaagtcacgggtcaccatcagcaaagataata |
| | | gcaagaaccaagtgtcacttaagctgtcatctgtgaccgccgctgacaccgccgtgtactattgtgc |
| | | caaacattactattacggagggtcttatgctatggactactggggacaggggaccctggtgactgtct |
| | | ctagccatcaccatcaccaccatcatcac |
| 99790 | 739 | MALPVTALLLPLALLLHAARPeivmtqspatlslspgeratlscrasqdiskyl |
| CAR6- | | nwyqqkpgqaprlliyhtsrlhsgiparfsgsgsgtdytltisslqpedfavyfcqqgntlpytfgq |
| Soluble | | gtkleikggggsggggsggggsggggsqvqlqesgpglvkpsetlsltctvsgvslpdygvswi |
| scFv-aa | | rqppgkglewigviwgsettyyqsslksrvtiskdnsknqvslklssvtaadtavyycakhyyy |
| | | ggsyamdywgqgtlvtvsshhhhhhhh |
| 104880 | 751 | atggccctccctgtcaccgccctgctgcttccgctggctcttctgctccacgccgctcggcccgaaa |
| CAR6- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagcggaggcggagggagccaggtccaactccaaga |
| | | aagcggaccgggtcttgtgaagccatcagaaactctttcactgacttgtactgtgagcggagtgtctc |
| | | tccccgattacggggtgtcttggatcagacagccaccggggaagggtctggaatggattggagtg |
| | | atttggggctctgagactacttactaccaatcatccctcaagtcacgcgtcaccatctcaaaggacaa |
| | | ctctaagaatcaggtgtcactgaaactgtcatctgtgaccgcagccgacaccgccgtgtactattgc |
| | | gctaagcattactattatggcgggagctacgcaatggattactggggacagggtactctggtcaccg |
| | | tgtccagcaccactaccccagcaccgaggccacccaccccggctcctaccatcgcctcccagcct |
| | | ctgtccctgcgtccggaggcatgtagacccgcagctggtggggccgtgcatacccggggtcttga |
| | | cttcgcctgcgatatctacatttgggcccctctggctggtacttgcggggtcctgctgctttcactcgtg |
| | | atcactctttactgtaagcgcggtcggaagaagctgctgtacatctttaagcaacccttcatgaggcct |
| | | gtgcagactactcaagaggaggacggctgttcatgccggttcccagaggaggaggaaggcggct |
| | | gcgaactgcgcgtgaaattcagccgcagcgcagatgctccagcctacaagcaggggcagaacca |
| | | gctctacaacgaactcaatcttggtcggagagaggagtacgacgtgctggacaagcggagagga |
| | | cgggacccagaaatgggcgggaagccgcgcagaaagaatccccaagagggcctgtacaacga |
| | | gctccaaaaggataagatggcagaagcctatagcgagattggtatgaaaggggaacgcagaaga |
| | | ggcaaaggccacgacggactgtaccagggactcagcaccgccaccaaggacacctatgacgct |
| | | cttcacatgcaggccctgccgcctcgg |
| 104880 | 763 | MALPVTALLLPLALLLHAARPeivmtqspatlslspgeratlscrasadiskyln |
| CAR6- | | wyqqkpgqaprlliyhtsrlhsgiparfsgsgsgtdytltisslqpedfavyfcqqgntlpytfgq |
| Full-aa | | gtkleikggggsggggsggggsggggsqvqlqesgpglvkpsetlsltctvsgvslpdygvswi |
| | | rqppgkglewigviwgsettyyqsslksrvtiskdnsknqvslklssvtaadtavyycakhyyy |
| | | ggsyamdywgqgtlvtvsstttpaprpptpaptiasqplslrpeacrpaaggavhtrgldfacdi |
| | | yiwaplagtcgvlllslvitlyckrgrkkllyifkqpfmrpvqttqeedgcscrfpeeeeggcelrv |
| | | kfsrsadapaykqgqnqlynelnlgrreeydvldkrrgrdpemggkprrknpqeglynelqkd |
| | | kmaeayseigmkgerrrgkghdglyqglstatkdtydalhmqalppr |

CAR 7

| | | |
|---|---|---|
| CAR7 | 716 | qvqlqesgpglvkpsetlsltctvsgvslpdygvswirqppgkglewigviwgsettyyssslks |
| scFv | | rvtiskdnsknqvslklssvtaadtavyycakhyyyggsyamdywgqgtlvtvssggggsgg |
| domain | | ggsggggsggggseivmtqspatlslspgeratlscrasqdiskylnwyqqkpgqaprlliyhts |
| | | rlhsgiparfsgsgsgtdytltisslqpedfavyfcqqgntlpytfgqgtkleik |
| 100796 | 728 | atggcactgcctgtcactgccctcctgctgcctctggccctccttctgcatgccgccaggccccaag |
| CAR7- | | tccagctgcaagagtcaggacccggactggtgaagccgtctgagactctctcactgacttgtaccgt |
| Soluble | | cagcggcgtgtccctccccgactacggagtgtcatggatccgccaacctcccgggaaagggcttg |
| scFv-nt | | aatggattggtgtcatctggggttctgaaaccacctactactcatcttccctgaagtccagggtgacc |
| | | atcagcaaggataattccaagaaccaggtcagccttaagctgtcatctgtgaccgctgctgacaccg |
| | | ccgtgtattactgcgccaagcactactattacggaggaagctacgctatggactattggggacagg |
| | | gcactctcgtgactgtgagcagcggcggtggagggtctggaggtggaggatccggtggtggtgg |
| | | gtcaggcggaggagggagcgagattgtgatgactcagtcaccagccaccctttctctttcacccgg |
| | | cgagagagcaaccctgagctgtagagccagccaggacatttctaagtacctcaactggtatcagca |
| | | aaaaccggggcaggcccctcgcctcctgatctaccatacctcacgccttcactctggtatccccgct |
| | | cggtttagcggatcaggatctggtaccgactacactctgaccatttccagcctgcagccagaagattt |
| | | cgcagtgtatttctgccagcagggcaatacccttccttacaccttcggtcagggaaccaagctcgaa |
| | | atcaagcaccatcaccatcatcaccaccat |
| 100796 | 740 | MALPVTALLLPLALLLHAARPqvqlqesgpglvkpsetlsltctvsgvslpdy |
| CAR7- | | gvswirqppgkglewigviwgsettyyssslksrvtiskdnsknqvslklssvtaadtavyycak |
| Soluble | | hyyyggsyamdywgqgtlvtvssggggsggggsggggsggggseivmtqspatlslspger |
| scFv-aa | | atlscrasqdiskylnwyqqkpgqaprlliyhtsrlhsgiparfsgsgsgtdytltisslqpedfavy |
| | | fcqqgntlpytfgqgtkleikhhhhhhhh |
| 104881 | 752 | atggctctgcccgtgaccgcactcctcctgccactggctctgctgcttcacgccgctcgcccacaag |
| CAR 7 | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactattcatcttccctgaagtcacgggtcacc |
| | | atttcaaaggataactcaaagaatcaagtgagcctcaagctctcatcagtcaccgccgctgacaccg |
| | | ccgtgtattactgtgccaagcattactactatggagggtcctacgccatggactactggggccaggg |
| | | aactctggtcactgtgtcatctggtggaggaggtagcggaggaggcgggagcggtggaggtggc |
| | | tccggaggtggcggaagcgaaatcgtgatgacccagagccctgcaaccctgtccctttctcccgg |
| | | ggaacgggctaccctttcttgtcgggcatcacaagatatctcaaaatacctcaattggtatcaacaga |
| | | agccgggacaggcccctaggcttcttatctaccacacctctcgcctgcatagcgggattcccgcac |
| | | gctttagcgggtctggaagcgggaccgactacactctgaccatctcatctctccagcccgaggactt |
| | | cgccgtctacttctgccagcagggtaacaccctgccgtacaccttcggccagggcaccaagcttga |
| | | gatcaaaaccactactcccgctccaaggccacccacccctgccccgaccatcgcctctcagccgct |
| | | ttccctgcgtccggaggcatgtagacccgcagctggtggggccgtgcatacccggggtcttgactt |
| | | cgcctgcgatatctacatttgggcccctctggctggtacttgcggggtcctgctgctttcactcgtgat |
| | | cactctttactgtaagcgcggtcggaagaagctgctgtacatctttaagcaacccttcatgaggcctg |
| | | tgcagactactcaagaggaggacggctgttcatgccggttcccagaggaggaggaaggcggctg |
| | | cgaactgcgcgtgaaattcagccgcagcgcagatgctccagcctacaagcaggggcagaaccag |
| | | ctctacaacgaactcaatcttggtcggagagaggagtacgacgtgctggacaagcggagaggac |
| | | gggacccagaaatgggcgggaagccgcgcagaaagaatccccaagagggcctgtacaacgag |
| | | ctccaaaaggataagatggcagaagcctatagcgagattggtatgaaaggggaacgcagaagag |
| | | gcaaaggccacgacggactgtaccagggactcagcaccgccaccaaggacacctatgacgctct |
| | | tcacatgcaggccctgccgcctcgg |
| 104881 | 764 | MALPVTALLLPLALLLHAARPqvqlqesgpglvkpsetlsltctvsgvslpdygv |
| CAR 7 | | swirqppgkglewigviwgsettyyssslksrvtiskdnsknqvslklssvtaadtavyycakh |
| Full-aa | | yyyggsyamdywgqgtlvtvssggggsggggsggggsggggseivmtqspatlslspgera |
| | | tlscrasqdiskylnwyqqkpgqaprlliyhtsrlhsgiparfsgsgsgtdytltisslqpedfav |
| | | yfcqqgntlpytfgqgtkleiktttpaprpptpaptiasqplslrpeacrpaaggavhtrgldfacd |
| | | iyiwaplagtcgvlllslvitlyckrgrkkllyifkqpfmrpvqttqeedgcscrfpeeeeggcelr |
| | | vkfsrsadapaykqgqnqlynelnlgrreeydvldkrrgrdpemggkprrknpqeglynelqk |
| | | dkmaeayseigmkgerrrgkghdglyqglstatkdtydalhmqalppr |

CAR 8

| | | |
|---|---|---|
| CAR8 | 717 | qvqlqesgpglvkpsetlsltctvsgvslpdygvswirqppgkglewigviwgsettyyqsslks |
| scFv | | rvtiskdnsknqvslklssvtaadtavyycakhyyyggsyamdywgqgtlvtvssggggsgg |
| domain | | ggsggggsggggseivmtqspatlslspgeratlscrasqdiskylnwyqqkpgqaprlliyhts |
| | | rlhsgiparfsgsgsgtdytltisslqpedfavyfcqqgntlpytfgqgtkleik |
| 100798 | 729 | atggcactgcctgtcactgccctcctgctgcctctggccctccttctgcatgccgccaggccccaag |
| CAR8- | | tccagctgcaagagtcaggacccggactggtgaagccgtctgagactctctcactgacttgtaccgt |
| Soluble | | cagcggcgtgtccctccccgactacggagtgtcatggatccgccaacctcccgggaaagggcttg |
| scFv-nt | | aatggattggtgtcatctggggttctgaaaccacctactaccagtcttccctgaagtccagggtgacc |
| | | atcagcaaggataattccaagaaccaggtcagccttaagctgtcatctgtgaccgctgctgacaccg |
| | | ccgtgtattactgcgccaagcactactattacggaggaagctacgctatggactattggggacagg |
| | | gcactctcgtgactgtgagcagcggcggtggagggtctggaggtggaggatccggtggtggtgg |
| | | gtcaggcggaggagggagcgagattgtgatgactcagtcaccagccaccctttctctttcacccgg |
| | | cgagagagcaaccctgagctgtagagccagccaggacatttctaagtacctcaactggtatcagca |
| | | aaaaccggggcaggcccctcgcctcctgatctaccatacctcacgccttcactctggtatccccgct |
| | | cggtttagcggatcaggatctggtaccgactacactctgaccatttccagcctgcagccagaagattt |
| | | cgcagtgtatttctgccagcagggcaatacccttccttacaccttcggtcagggaaccaagctcgaa |
| | | atcaagcaccatcaccatcatcatcaccac |
| 100798 | 741 | MALPVTALLLPLALLLHAARPqvqlqesgpglvkpsetlsltctvsgvslpdy |
| CAR8- | | gvswirqppgkglewigviwgsettyyqsslksrvtiskdnsknqvslklssvtaadtavyyca |
| Soluble | | khyyyggsyamdywgqgtlvtvssggggsggggsggggsggggseivmtqspatlslspge |
| scFv-aa | | ratlscrasqdiskylnwyqqkpgqaprlliyhtsrlhsgiparfsgsgsgtdytltisslqpedfav |
| | | yfcqqgntlpytfgqgtkleikhhhhhhhh |
| 104882 | 753 | atggctctgcccgtgaccgcactcctcctgccactggctctgctgcttcacgccgctcgcccacaag |
| CAR 8- | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactatcaatcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccggaggcggtgggtcagaaatcgtgatgacccagagccctgcaaccctgtccctttctcccgg |
| | | ggaacgggctaccctttcttgtcgggcatcacaagatatctcaaaatacctcaattggtatcaacaga |
| | | agccgggacaggcccctaggcttcttatctaccacacctctcgcctgcatagcgggattcccgcac |
| | | gctttagcgggtctggaagcgggaccgactacactctgaccatctcatctctccagcccgaggactt |
| | | cgccgtctacttctgccagcagggtaacaccctgccgtacaccttcggccagggcaccaagcttga |
| | | gatcaaaaccactactcccgctccaaggccacccacccctgccccgaccatcgcctctcagccgct |
| | | ttccctgcgtccggaggcatgtagacccgcagctggtggggccgtgcatacccggggtcttgactt |
| | | cgcctgcgatatctacatttgggcccctctggctggtacttgcggggtcctgctgctttcactcgtgat |
| | | cactctttactgtaagcgcggtcggaagaagctgctgtacatctttaagcaacccttcatgaggcctg |
| | | tgcagactactcaagaggaggacggctgttcatgccggttcccagaggaggaggaaggcggctg |
| | | cgaactgcgcgtgaaattcagccgcagcgcagatgctccagcctacaagcaggggcagaaccag |
| | | ctctacaacgaactcaatcttggtcggagagaggagtacgacgtgctggacaagcggagaggac |
| | | gggacccagaaatgggcgggaagccgcgcagaaagaatccccaagagggcctgtacaacgag |
| | | ctccaaaaggataagatggcagaagcctatagcgagattggtatgaaaggggaacgcagaagag |
| | | gcaaaggccacgacggactgtaccagggactcagcaccgccaccaaggacacctatgacgctct |
| | | tcacatgcaggccctgccgcctcgg |
| 104882 | 765 | MALPVTALLLPLALLLHAARPqvqlqesgpglvkpsetlsltctvsgvslpdygv |
| CAR 8- | | swirqppgkglewigviwgsettyyqsslksrvtiskdnsknqvslklssvtaadtavyycakh |
| Full-aa | | yyyggsyamdywgqgtlvtvssggggsggggsggggsggggseivmtqspatlslspgera |
| | | tlscrasqdiskylnwyqqkpgqaprlliyhtsrlhsgiparfsgsgsgtdytltisslqpedfav |
| | | yfcqqgntlpytfgqgtkleiktttpaprpptpaptiasqplslrpeacrpaaggavhtrgldfacd |
| | | iyiwaplagtcgvlllslvitlyckrgrkkllyifkqpfmrpvqttqeedgcscrfpeeeeggcelr |
| | | vkfsrsadapaykqgqnqlynelnlgrreeydvldkrrgrdpemggkprrknpqeglynelqk |
| | | dkmaeayseigmkgerrrgkghdglyqglstatkdtydalhmqalppr |

CAR 9

| | | |
|---|---|---|
| CAR9 | 718 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsggggsqvqlq |
| domain | | esgpglvkpsetlsltctvsgvslpdygvswirqppgkglewigviwgsettyynsslksrvtisk |
| | | dnsknqvslklssvtaadtavyycakhyyyggsyamdywgqgtlvtvss |
| 99789 | 730 | atggccctcccagtgaccgctctgctgctgcctctcgcacttcttctccatgccgctcggcctgagat |
| CAR9- | | cgtcatgacccaaagccccgctaccctgtccctgtcacccggcgagagggcaaccctttcatgcag |
| Soluble | | ggccagccaggacatttctaagtacctcaactggtatcagcagaagccagggcaggctcctcgcct |
| scFv-nt | | gctgatctaccacaccagccgcctccacagcggtatccccgccagattttccgggagcgggtctgg |
| | | aaccgactacaccctcaccatctcttctctgcagcccgaggatttcgccgtctatttctgccagcagg |
| | | ggaatactctgccgtacaccttcggtcaaggtaccaagctggaaatcaagggaggcggaggatca |
| | | ggcggtggcggaagcggaggaggtggctccggaggaggaggttcccaagtgcagcttcaagaa |
| | | tcaggacccggacttgtgaagccatcagaaaccctctccctgacttgtaccgtgtccggtgtgagcc |
| | | tccccgactacggagtctcttggattcgccagcctccggggaagggtcttgaatggattggggtgat |
| | | ttggggatcagagactacttactacaattcatcacttaagtcacgggtcaccatcagcaaagataata |
| | | gcaagaaccaagtgtcacttaagctgtcatctgtgaccgccgctgacaccgccgtgtactattgtgc |
| | | caaacattactattacggagggtcttatgctatggactactggggacaggggaccctggtgactgtct |
| | | ctagccatcaccatcaccaccatcatcac |
| 99789 | 742 | MALPVTALLLPLALLLHAARPeivmtqspatlslspgeratlscrasqdiskyl |
| CAR9- | | nwyqqkpgqaprlliyhtsrlhsgiparfsgsgsgtdytltisslqpedfavyfcqqgntlpytfgq |
| Soluble | | gtkleikggggsggggsggggsggggsqvqlqesgpglvkpsetlsltctvsgvslpdygvswi |
| scFv-aa | | rqppgkglewigviwgsettyynsslksrvtiskdnsknqvslklssvtaadtavyycakhyyy |
| | | ggsyamdywgqgtlvtvsshhhhhhhh |
| 105974 | 754 | atggccctccctgtcaccgccctgctgcttccgctggctcttctgctccacgccgctcggcccgaaa |
| CAR 9- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagcggaggcggtgggagccaggtccaactccaagaa |
| | | agcggaccgggtcttgtgaagccatcagaaactctttcactgacttgtactgtgagcggagtgtctct |
| | | ccccgattacggggtgtcttggatcagacagccaccggggaagggtctggaatggattggagtga |
| | | tttggggctctgagactacttactacaactcatccctcaagtcacgcgtcaccatctcaaaggacaac |
| | | tctaagaatcaggtgtcactgaaactgtcatctgtgaccgcagccgacaccgccgtgtactattgcg |
| | | ctaagcattactattatggcgggagctacgcaatggattactggggacagggtactctggtcaccgt |
| | | gtccagcaccactaccccagcaccgaggccacccaccccggctcctaccatcgcctcccagcctc |
| | | tgtccctgcgtccggaggcatgtagacccgcagctggtggggccgtgcatacccggggtcttgact |
| | | tcgcctgcgatatctacatttgggcccctctggctggtacttgcggggtcctgctgctttcactcgtgat |
| | | cactctttactgtaagcgcggtcggaagaagctgctgtacatctttaagcaacccttcatgaggcctg |
| | | tgcagactactcaagaggaggacggctgttcatgccggttcccagaggaggaggaaggcggctg |
| | | cgaactgcgcgtgaaattcagccgcagcgcagatgctccagcctacaagcaggggcagaaccag |
| | | ctctacaacgaactcaatcttggtcggagagaggagtacgacgtgctggacaagcggagaggac |
| | | gggacccagaaatgggcgggaagccgcgcagaaagaatccccaagagggcctgtacaacgag |
| | | ctccaaaaggataagatggcagaagcctatagcgagattggtatgaaaggggaacgcagaagag |
| | | gcaaaggccacgacggactgtaccagggactcagcaccgccaccaaggacacctatgacgctct |
| | | tcacatgcaggccctgccgcctcgg |
| 105974 | 766 | MALPVTALLLPLALLLHAARPeivrntqspatlslspgeratlscrasqdiskyln |
| CAR9- | | wyqqkpgqaprlliyhtsrlhsgiparfsgsgsgtdytltisslqpedfavyfcqqgntlpytfgq |
| Full-aa | | gtkleikggggsggggsggggsggggsqvqlqesgpglvkpsetlsltctvsgvslpdygvswi |
| | | rqppgkglewigviwgsettyynsslksrvtiskdnsknqvslklssvtaadtavyycakhyyy |
| | | ggsyamdywgqgtlvtvsstttpaprpptpaptiasqplslrpeacrpaaggavhtrgldfacdi |
| | | yiwaplagtcgvlllslvitlyckrgrkkllyifkqpfmrpvqttqeedgcscrfpeeeeggcelrv |
| | | kfsrsadapaykqgqnqlynelnlgrreeydvldkrrgrdpemggkprrknpqeglynelqkd |
| | | kmaeayseigmkgerrrgkghdglyqglstatkdtydalhmqalppr |

CAR10

| | | |
|---|---|---|
| CAR10 | 719 | qvqlqesgpglvkpsetlsltctvsgvslpdygvswirqppgkglewigviwgsettyynsslks |
| scFv | | rvtiskdnsknqvslklssvtaadtavyycakhyyyggsyamdywgqgtlvtvssggggsgg |
| domain | | ggsggggsggggseivmtqspatlslspgeratlscrasqdiskylnwyqqkpgqaprlliyhts |
| | | rlhsgiparfsgsgsgtdytltisslqpedfavyfcqqgntlpytfgqgtkleik |
| 100796 | 731 | atggcactgcctgtcactgccctcctgctgcctctggccctccttctgcatgccgccaggccccaag |
| CAR10- | | tccagctgcaagagtcaggacccggactggtgaagccgtctgagactctctcactgacttgtaccgt |
| Soluble | | cagcggcgtgtccctccccgactacggagtgtcatggatccgccaacctcccgggaaagggcttg |
| scFv-nt | | aatggattggtgtcatctggggttctgaaaccacctactacaactcttccctgaagtccagggtgacc |
| | | atcagcaaggataattccaagaaccaggtcagccttaagctgtcatctgtgaccgctgctgacaccg |
| | | ccgtgtattactgcgccaagcactactattacggaggaagctacgctatggactattggggacagg |
| | | gcactctcgtgactgtgagcagcggcggtggagggtctggaggtggaggatccggtggtggtgg |
| | | gtcaggcggaggagggagcgagattgtgatgactcagtcaccagccaccctttctctttcacccgg |
| | | gcaaaggccacgacggactgtaccagggactcagcaccgccaccaaggacacctatgacgctct |
| | | tcacatgcaggccctgccgcctcgg |
| 105975 | 767 | MALPVTALLLPLALLLHAARPEIVMTQSPATLSLSPGERATLSC |
| CAR 10 | | RASQDISKYLNWYQQKPGQAPRLLIYHTSRLHSGIPARFSGSG |
| Full-aa | | SGTDYTLTISSLQPEDFAVYFCQQGNTLPYTFGQGTKLEIKGG |
| | | GGSGGGGSGGGGSGGGGSQVQLQESGPGLVKPSETLSLTCTVS |
| | | GVSLPDYGVSWIRQPPGKGLEWIGVIWGSETTYYNSSLKSRV |
| | | TISKDNSKNQVSLKLSSVTAADTAVYYCAKHYYYGGSYAMD |
| | | YWGQGTLVTVSSTTTPAPRPPTPAPTIASQPLSLRPEACRPAAG |
| | | GAVHTRGLDFACDIYIWAPLAGTCGVLLLSLVITLYCKRGRKK |
| | | LLYIFKQPFMRPVQTTQEEDGCSCRFPEEEEGGCELRVKFSRSA |
| | | DAPAYKQGQNQLYNELNLGRREEYDVLDKRRGRDPEMGGKP |
| | | RRKNPQEGLYNELQKDKMAEAYSEIGMKGERRRGKGHDGLY |
| | | QGLSTATKDTYDALHMQALPPR |

CAR11

| | | |
|---|---|---|
| CAR11 | 720 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsqvqlqesgpg |
| domain | | lvkpsetlsltctvsgvslpdygvswirqppgkglewigviwgsettyynsslksrvtiskdnskn |
| | | qvslklssvtaadtavyycakhyyyggsyamdywgqgtlvtvss |
| 103101 | 732 | Atggccctccctgtcaccgccctgctgcttccgctggctcttctgctccacgccgctcggcccgaa |
| CAR11- | | attgtgatgacccagtcacccgccactcttagcctttcacccggtgagcgcgcaaccctgtcttgca |
| Soluble | | gagcctcccaagacatctcaaaataccttaattggtatcaacagaagcccggacaggctcctcgcct |
| scFv-nt | | tctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactacaattcatccctcaagtcacgcgtcaccatctcaaaggacaactctaagaatcaggtgtca |
| | | ctgaaactgtcatctgtgaccgcagccgacaccgccgtgtactattgcgctaagcattactattatgg |
| | | cgggagctacgcaatggattactggggacagggtactctggtcaccgtgtccagccaccaccatc |
| | | atcaccatcaccat |
| 103101 | 744 | MALPVTALLLPLALLLHAARPeivmtqspatlslspgeratlscrasqdiskyl |
| CAR11- | | nwyqqkpgqaprlliyhtsrlhsgiparfsgsgsgtdytltisslqpedfavyfcqqgntlpytfgq |
| Soluble | | gtkleikggggsggggsggggsqvqlqesgpglvkpsetlsltctvsgvslpdygvswirqppg |
| scFv-aa | | kglewigviwgsettyynsslksrvtiskdnsknqvslklssvtaadtavyycakhyyyggsya |
| | | mdywgqgtlvtvsshhhhhhhh |
| 105976 | 756 | atggctctgcccgtgaccgcactcctcctgccactggctctgctgcttcacgccgctcgcccacaag |
| CAR 11 | | tccagcttcaagaatcagggcctggtctggtgaagccatctgagactctgtccctcacttgcaccgtg |
| Full-nt | | agcggagtgtccctcccagactacggagtgagctggattagacagcctcccggaaagggactgg |
| | | agtggatcggagtgatttggggtagcgaaaccacttactataactcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccggaggtggcggaagcgaaatcgtgatgacccagagccctgcaaccctgtccctttctcccg |
| | | gggaacgggctaccctttcttgtcgggcatcacaagatatctcaaaatacctcaattggtatcaacag |
| | | aagccgggacaggcccctaggcttcttatctaccacacctctcgcctgcatagcgggattcccgca |
| | | cgctttagcgggtctggaagcgggaccgactacactctgaccatctcatctctccagcccgaggac |
| | | ttcgccgtctacttctgccagcagggtaacaccctgccgtacaccttcggccagggcaccaagcttg |
| | | agatcaaaaccactactcccgctccaaggccacccacccctgccccgaccatcgcctctcagccg |
| | | ctttccctgcgtccggaggcatgtagacccgcagctggtggggccgtgcatacccggggtcttgac |
| | | ttcgcctgcgatatctacatttgggcccctctggctggtacttgcggggtcctgctgctttcactcgtga |
| | | tcactctttactgtaagcgcggtcggaagaagctgctgtacatctttaagcaacccttcatgaggcct |
| | | gtgcagactactcaagaggaggacggctgttcatgccggttcccagaggaggaggaaggcggct |
| | | gcgaactgcgcgtgaaattcagccgcagcgcagatgctccagcctacaagcaggggcagaacca |
| | | gctctacaacgaactcaatcttggtcggagagaggagtacgacgtgctggacaagcggagagga |
| | | cgggacccagaaatgggcgggaagccgcgcagaaagaatccccaagagggcctgtacaacga |
| | | gctccaaaaggataagatggcagaagcctatagcgagattggtatgaaaggggaacgcagaaga |
| | | ggcaaaggccacgacggactgtaccagggactcagcaccgccaccaaggacacctatgacgct |
| | | cttcacatgcaggccctgccgcctcgg |
| 105976 | 768 | MALPVTALLLPLALLLHAARPQVQLQESGPGLVKPSETLSLTC |
| CAR 11 | | TVSGVSLPDYGVSWIRQPPGKGLEWIGVIWGSETTYYNSSLK |
| Full-aa | | SRVTISKDNSKNQVSLKLSSVTAADTAVYYCAKHYYYGGSYA |
| | | MDYWGQGTLVTVSSGGGGSGGGGSGGGGSGGGGSEIVMTQS |
| | | PATLSLSPGERATLSCRASQDISKYLNWYQQKPGQAPRLLIYH |
| | | TSRLHSGIPARFSGSGSGTDYTLTISSLQPEDFAVYFCQQGNTL |
| | | PYTFGQGTKLEIKTTTPAPRPPTPAPTIASQPLSLRPEACRPAAG |
| | | GAVHTRGLDFACDIYIWAPLAGTCGVLLLSLVITLYCKRGRKK |
| | | LLYIFKQPFMRPVQTTQEEDGCSCRFPEEEEGGCELRVKFSRSA |
| | | DAPAYKQGQNQLYNELNLGRREEYDVLDKRRGRDPEMGGKP |
| | | RRKNPQEGLYNELQKDKMAEAYSEIGMKGERRRGKGHDGLY |
| | | QGLSTATKDTYDALHMQALPPR |

CAR12

| | | |
|---|---|---|
| CAR12 | 721 | qvqlqesgpglvkpsetlsltctvsgvslpdygvswirqppgkglewigviwgsettyynsslks |
| scFv | | rvtiskdnsknqvslklssvtaadtavyycakhyyyggsyamdywgqgtlvtvssggggsgg |
| domain | | ggsggggseivmtqspatlslspgeratlscrasqdiskylnwyqqkpgqaprlliyhtsrlhsgi |
| | | parfsgsgsgtdytltisslqpedfavyfcqqgntlpytfgqgtkleik |
| 103104 | 733 | atggctctgcccgtgaccgcactcctcctgccactggctctgctgcttcacgccgctcgcccacaag |
| CAR12- | | tccagcttcaagaatcagggcctggtctggtgaagccatctgagactctgtccctcacttgcaccgtg |
| Soluble | | agcggagtgtccctcccagactacggagtgagctggattagacagcctcccggaaagggactgg |
| scFv-nt | | agtggatcggagtgatttggggtagcgaaaccacttactataactcttccctgaagtcacgggtcac |
| | | catttcaaaggataactcaaagaatcaagtgagcctcaagctctcatcagtcaccgccgctgacacc |
| | | gccgtgtattactgtgccaagcattactactatggagggtcctacgccatggactactggggccagg |
| | | gaactctggtcactgtgtcatctggtggaggaggtagcggaggaggcgggagcggtggaggtgg |
| | | ctccgaaatcgtgatgacccagagccctgcaaccctgtccctttctcccggggaacgggctaccctt |
| | | tcttgtcgggcatcacaagatatctcaaaatacctcaattggtatcaacagaagccgggacaggccc |
| | | ctaggcttcttatctaccacacctctcgcctgcatagcgggattcccgcacgctttagcgggtctgga |
| | | agcgggaccgactacactctgaccatctcatctctccagcccgaggacttcgccgtctacttctgcc |
| | | agcagggtaacaccctgccgtacaccttcggccagggcaccaagcttgagatcaaacatcaccac |
| | | catcatcaccatcac |
| 103104 | 745 | MALPVTALLLPLALLLHAARPqvqlqesgpglvkpsetlsltctvsgvslpdy |
| CAR12- | | gvswirqppgkglewigviwgsettyynsslksrvtiskdnsknqvslklssvtaadtavyyca |
| Soluble | | khyyyggsyamdywgqgtlvtvssggggsggggsggggseivmtqspatlslspgeratlscr |
| scFv-aa | | asqdiskylnwyqqkpgqaprlliyhtsrlhsgiparfsgsgsgtdytltisslqpedfavyfcqqg |
| | | ntlpytfgqgtkleikhhhhhhhh |
| 105977 | 757 | atggccctccctgtcaccgccctgctgcttccgctggctcttctgctccacgccgctcggcccgaaa |
| CAR12- | | ttgtgatgacccagtcacccgccactcttagcctttcacccggtgagcgcgcaaccctgtcttgcag |
| Full-nt | | agcctcccaagacatctcaaaataccttaattggtatcaacagaagcccggacaggctcctcgcctt |
| | | ctgatctaccacaccagccggctccattctggaatccctgccaggttcagcggtagcggatctggg |
| | | accgactacaccctcactatcagctcactgcagccagaggacttcgctgtctatttctgtcagcaagg |
| | | gaacaccctgccctacacctttggacagggcaccaagctcgagattaaaggtggaggtggcagcg |
| | | gaggaggtgggtccggcggtggaggaagccaggtccaactccaagaaagcggaccgggtcttg |
| | | tgaagccatcagaaactctttcactgacttgtactgtgagcggagtgtctctccccgattacggggtgt |
| | | cttggatcagacagccaccggggaagggtctggaatggattggagtgatttggggctctgagacta |
| | | cttactacaactcatccctcaagtcacgcgtcaccatctcaaaggacaactctaagaatcaggtgtca |
| | | ctgaaactgtcatctgtgaccgcagccgacaccgccgtgtactattgcgctaagcattactattatgg |
| | | cgggagctacgcaatggattactggggacagggtactctggtcaccgtgtccagcaccactaccc |
| | | cagcaccgaggccacccaccccggctcctaccatcgcctcccagcctctgtccctgcgtccggag |
| | | gcatgtagacccgcagctggtggggccgtgcatacccggggtcttgacttcgcctgcgatatctac |
| | | atttgggcccctctggctggtacttgcggggtcctgctgctttcactcgtgatcactctttactgtaagc |
| | | gcggtcggaagaagctgctgtacatctttaagcaacccttcatgaggcctgtgcagactactcaaga |
| | | ggaggacggctgttcatgccggttcccagaggaggaggaaggcggctgcgaactgcgcgtgaa |
| | | attcagccgcagcgcagatgctccagcctacaagcaggggcagaaccagctctacaacgaactc |
| | | aatcttggtcggagagaggagtacgacgtgctggacaagcggagaggacgggacccagaaatg |
| | | ggcgggaagccgcgcagaaagaatccccaagagggcctgtacaacgagctccaaaaggataag |
| | | atggcagaagcctatagcgagattggtatgaaaggggaacgcagaagaggcaaaggccacgac |
| | | ggactgtaccagggactcagcaccgccaccaaggacacctatgacgctcttcacatgcaggccct |
| | | gccgcctcgg |
| 105977 | 769 | MALPVTALLLPLALLLHAARPEIVMTQSPATLSLSPGERATLSC |
| CAR12- | | RASQDISKYLNWYQQKPGQAPRLLIYHTSRLHSGIPARFSGSG |
| Full-aa | | SGTDYTLTISSLQPEDFAVYFCQQGNTLPYTFGQGTKLEIKGG |
| | | GGSGGGGSGGGGSQVQLQESGPGLVKPSETLSLTCTVSGVSLP |
| | | DYGVSWIRQPPGKGLEWIGVIWGSETTYYNSSLKSRVTISKD |
| | | NSKNQVSLKLSSVTAADTAVYYCAKHYYYGGSYAMDYWGQ |
| | | GTLVTVSSTTTPAPRPPTPAPTIASQPLSLRPEACRPAAGGAVH |
| | | TRGLDFACDIYIWAPLAGTCGVLLLSLVITLYCKRGRKKLLYIF |
| | | KQPFMRPVQTTQEEDGCSCRFPEEEEGGCELRVKFSRSADAPA |
| | | YKQGQNQLYNELNLGRREEYDVLDKRRGRDPEMGGKPRRKN |
| | | PQEGLYNELQKDKMAEAYSEIGMKGERRRGKGHDGLYQGLS |
| | | TATKDTYDALHMQALPPR |

TABLE 14A
Murine CD19 CAR Constructs
CTL019

| | | |
|---|---|---|
| CTL019- | 770 | Atggccctgcccgtcaccgctctgctgctgccccttgctctgcttcttcatgcagcaaggccggaca |
| Soluble | | tccagatgacccaaaccacctcatccctctctgcctctcttggagacagggtgaccatttcttgtcgc |
| scFv- | | gccagccaggacatcagcaagtatctgaactggtatcagcagaagccggacggaaccgtgaagc |
| Histag-nt | | tcctgatctaccatacctctcgcctgcatagcggcgtgccctcacgcttctctggaagcggatcagg |
| | | aaccgattattctctcactatttcaaatcttgagcaggaagatattgccacctatttctgccagcagggt |
| | | aataccctgccctacaccttcggaggagggaccaagctcgaaatcaccggtggaggaggcagcg |
| | | gcggtggagggtctggtggaggtggttctgaggtgaagctgcaagaatcaggccctggacttgtg |
| | | gccccttcacagtccctgagcgtgacttgcaccgtgtccggagtctccctgcccgactacggagtgt |
| | | catggatcagacaacctccacggaaaggactggaatggctcggtgtcatctggggtagcgaaact |
| | | acttactacaattcagccctcaaaagcaggctgactattatcaaggacaacagcaagtcccaagtctt |
| | | tcttaagatgaactcactccagactgacgacaccgcaatctactattgtgctaagcactactactacg |
| | | gaggatcctacgctatggattactggggacaaggtacttccgtcactgtctcttcacaccatcatcac |
| | | catcaccatcac |
| CTL019- | 771 | MALPVTALLLPLALLLHAARPdiqmtqttsslsaslgdrvtiscrasqdiskyl |
| Soluble | | nwyqqkpdgtvklliyhtsrlhsgvpsrfsgsgsgtdysltisnleqediatyfcqqgntlpytfgg |
| scFv- | | gtkleitggggsggggsggggsevklqesgpglvapsqslsvtctvsgvslpdygvswirqppr |
| Histag-aa | | kglewlgviwgsettyynsalksrltiikdnsksqvflkmnslqtddtaiyycakhyyyggsya |
| | | mdywgqgtsvtvsshhhhhhhh |
| CTL019 | 772 | atggccttaccagtgaccgccttgctcctgccgctggccttgctgctccacgccgccaggccggac |
| Full-nt | | atccagatgacacagactacatcctccctgtctgcctctctgggagacagagtcaccatcagttgca |
| | | gggcaagtcaggacattagtaaatatttaaattggtatcagcagaaaccagatggaactgttaaactc |
| | | ctgatctaccatacatcaagattacactcaggagtcccatcaaggttcagtggcagtgggtctggaa |
| | | cagattattctctcaccattagcaacctggagcaagaagatattgccacttacttttgccaacagggta |
| | | atacgcttccgtacacgttcggaggggggaccaagctggagatcacaggtggcggtggctcggg |
| | | cggtggtgggtcgggtggcggcggatctgaggtgaaactgcaggagtcaggacctggcctggtg |
| | | gcgccctcacagagcctgtccgtcacatgcactgtctcaggggtctcattacccgactatggtgtaa |
| | | gctggattcgccagcctccacgaaagggtctggagtggctgggagtaatatggggtagtgaaacc |
| | | acatactataattcagctctcaaatccagactgaccatcatcaaggacaactccaagagccaagtttt |
| | | cttaaaaatgaacagtctgcaaactgatgacacagccatttactactgtgccaaacattattactacgg |
| | | tggtagctatgctatggactactggggccaaggaacctcagtcaccgtctcctcaaccacgacgcc |
| | | agcgccgcgaccaccaacaccggcgcccaccatcgcgtcgcagcccctgtccctgcgcccaga |
| | | ggcgtgccggccagcggcggggggcgcagtgcacacgagggggctggacttcgcctgtgatat |
| | | ctacatctgggcgcccttggccgggacttgtggggtccttctcctgtcactggttatcaccctttactg |
| | | caaacggggcagaaagaaactcctgtatatattcaaacaaccatttatgagaccagtacaaactact |
| | | caagaggaagatggctgtagctgccgatttccagaagaagaagaaggaggatgtgaactgagagt |
| | | gaagttcagcaggagcgcagacgcccccgcgtacaagcagggccagaaccagctctataacga |
| | | gctcaatctaggacgaagagaggagtacgatgttttggacaagagacgtggccgggaccctgaga |
| | | tggggggaaagccgagaaggaagaaccctcaggaaggcctgtacaatgaactgcagaaagata |
| | | agatggcggaggcctacagtgagattgggatgaaaggcgagcgccggaggggcaaggggcac |
| | | gatggcctttaccagggtctcagtacagccaccaaggacacctacgacgcccttcacatgcaggcc |
| | | ctgccccctcgc |
| CTL019 | 773 | MALPVTALLLPLALLLHAARPdiqmtqttsslsaslgdrvtiscrasqdiskylnw |
| Full-aa | | yqqkpdgtvklliyhtsrlhsgvpsrfsgsgsgtdysltisnleqediatyfcqqgntlpytfgggtk |
| | | leitggggsggggsggggsevklqesgpglvapsqslsvtctvsgvslpdygvswirqpprkgl |
| | | ewlgviwgsettyynsalksrltiikdnsksqvflkmnslqtddtaiyycakhyyyggsyamd |
| | | ywgqgtsvtvsstttpaprpptpaptiasqplslrpeacrpaaggavhtrgldfacdiyiwaplagt |
| | | cgvlllslvitlyckrgrkkllyifkqpfmrpvqttqeedgcscrfpeeeeggcelrvkfsrsadap |
| | | aykqgqnqlynelnlgrreeydvldkrrgrdpemggkprrknpqeglynelqkdkmaeayse |
| | | igmkgerrrgkghdglyqglstatkdtydalhmqalppr |
| CTL019 | 774 | Diqmtqttsslsaslgdrvtiscrasqdiskylnwyqqkpdgtvklliyhtsrlhsgvpsrfsgsgs |
| scFv | | gtdysltisnleqediatyfcqqgntlpytfgggtkleitggggsggggsggggsevklqesgpgl |
| domain | | vapsqslsvtctvsgvslpdygvswirqpprkglewlgviwgsettyynsalksrltiikdnsksq |
| | | vflkmnslqtddtaiyycakhyyyggsyamdywgqgtsvtvss |
| mCAR1 | 775 | QVQLLESGAELVRPGSSVKISCKASGYAFSSYWMNWVKQRPG |
| scFv | | QGLEWIGQIYPGDGDTNYNGKFKGQATLTADKSSSTAYMQLS |
| | | GLTSEDSAVYSCARKTISSVVDFYFDYWGQGTTVTGGGSGGG |
| | | SGGGSGGGSELVLTQSPKFMSTSVGDRVSVTCKASQNVGTNV |
| | | AWYQQKPGQSPKPLIYSATYRNSGVPDRFTGSGSGTDFTLTIT |
| | | NVQSKDLADYFCQYNRYPYTSFFFTKLEIKRRS |
| mCAR1 | 776 | QVQLLESGAELVRPGSSVKISCKASGYAFSSYWMNWVKQRPG |
| Full-aa | | QGLEWIGQIYPGDGDTNYNGKFKGQATLTADKSSSTAYMQLS |
| | | GLTSEDSAVYSCARKTISSVVDFYFDYWGQGTTVTGGGSGGG |
| | | SGGGSGGGSELVLTQSPKFMSTSVGDRVSVTCKASQNVGTNV |
| | | AWYQQKPGQSPKPLIYSATYRNSGVPDRFTGSGSGTDFTLTIT |
| | | NVQSKDLADYFCQYNRYPYTSFFFTKLEIKRRSKIEVMYPPPYL |
| | | DNEKSNGTIIHVKGKHLCPSPLFPGPSKPFWVLVVVGGVLACY |
| | | SLLVTVAFIIFWVRSKRSRLLHSDYMNMTPRRPGPTRKHYQPY |
| | | APPRDFAAYRSRVKFSRSADAPAYQQGQNQLYNELNLGRREE |
| | | YDVLDKRRGRDPEMGGKPRRKNPQEGLYNELQKDKMAEAYS |
| | | EIGMKGERRRGKGHDGLYQGLSTATKDTYDALHMQALPPR |
| mCAR2 | 777 | DIQMTQTTSSLSASLGDRVTISCRASQDISKYLNWYQQKPDGT |
| scFv | | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSSE |
| mCAR2 | 778 | DIQMTQTTSSLSASLGDRVTISCRASQDISKYLNWYQQKPDGT |
| CAR-aa | | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSSESKYGPPCPPCPMF |
| | | WVLVVVGGVLACYSLLVTVAFIIFWVKRGRKKLLYIFKQPFM |
| | | RPVQTTQEEDGCSCRFEEEEGGCELRVKFSRSADAPAYQQGQ |
| | | NQLYNELNLGRREEYDVLDKRRGRDPEMGGKPRRKNPQEGL |
| | | YNELQKDKMAEAYSEIGMKGERRRGKGHDGLYQGLSTATKD |
| | | TYDALHMQALPPRL |
| mCAR2 | 779 | DIQMTQTT SSLSASLGDR VTISCRASQD ISKYLNWYQQ |
| Full-aa | | KPDGTVKLLI YHTSRLHSGV PSRFSGSGSG TDYSLTISNL |
| | | EQEDIATYFC QQGNTLPYTF GGGTKLEITG STSGSGKPGS |
| | | GEGSTKGEVK LQESGPGLVA PSQSLSVTCT VSGVSLPDYG |
| | | VSWIRQPPRK GLEWLGVIWG SETTYYNSAL KSRLTIIKDN |
| | | SKSQVFLKMN SLQTDDTAIY YCAKHYYYGG |
| | | SYAMDYWGQG TSVTVSSESK YGPPCPPCPM FWVLVVVGGV |
| | | LACYSLLVTV |
| | | AFIIFWVKRG RKKLLYIFKQ PFMRPVQTTQ EEDGCSCRFE |
| | | EEEGGCELRV KFSRSADAPA YQQGQNQLYN ELNLGRREEY |
| | | DVLDKRRGRD PEMGGKPRRK NPQEGLYNEL QKDKMAEAYS |
| | | EIGMKGERRR GKGHDGLYQG LSTATKDTYD ALHMQALPPR |
| | | LEGGGEGRGS LLTCGDVEEN PGPRMLLLVT SLLLCELPHP |
| | | AFLLIPRKVC NGIGIGEFKD SLSINATNIK HFKNCTSISG |
| | | DLHILPVAFR GDSFTHTPPL DPQELDILKT VKEITGFLLI |
| | | QAWPENRTDL HAFENLEIIR |
| | | GRTKQHGQFS LAVVSLNITS LGLRSLKEIS DGDVIISGNK |
| | | NLCYANTINW KKLFGTSGQK TKIISNRGEN SCKATGQVCH |
| | | ALCSPEGCWG PEPRDCVSCR NVSRGRECVD KCNLLEGEPR |
| | | EFVENSECIQ CHPECLPQAM NITCTGRGPD NCIQCAHYID |
| | | GPHCVKTCPA GVMGENNTLV WKYADAGHVC |
| | | HLCHPNCTYG CTGPGLEGCP TNGPKIPSIA TGMVGALLLL |
| | | LVVALGIGLF M |
| mCAR3 | 780 | DIQMTQTTSSLSASLGDRVTISCRASQDISKYLNWYQQKPDGT |
| scFv | | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSS |
| mCAR3 | 781 | DIQMTQTTSSLSASLGDRVTISCRASQDISKYLNWYQQKPDGT |
| Full-aa | | VKLLIYHTSRLHSGVPSRFSGSGSGTDYSLTISNLEQEDIATYFC |
| | | QQGNTLPYTFGGGTKLEITGSTSGSGKPGSGEGSTKGEVKLQE |
| | | SGPGLVAPSQSLSVTCTVSGVSLPDYGVSWIRQPPRKGLEWLG |
| | | VIWGSETTYYNSALKSRLTIIKDNSKSQVFLKMNSLQTDDTAIY |
| | | YCAKHYYYGGSYAMDYWGQGTSVTVSSAAAIEVMYPPPYLD |
| | | NEKSNGTIIHVKGKHLCPSPLFPGPSKPFWVLVVVGGVLACYS |
| | | LLVTVAFIIFWVRSKRSRLLHSDYMNMTPRRPGPTRKHYQPYA |
| | | PPRDFAAYRSRVKFSRSADAPAYQQGQNQLYNELNLGRREEY |
| | | DVLDKRRGRDPEMGGKPRRKNPQEGLYNELQKDKMAEAYSE |
| | | IGMKGERRRGKGHDGLYQGLSTATKDTYDALHMQALPPR |
| SSJ25-C1 | 791 | QVQLLESGAELVRPGSSVKISCKASGYAFSSYWMNWVKQRPG |
| VH | | QGLEWIGQIYPGDGDTNYNGKFKGQATLTADKSSSTAYMQLS |
| sequence | | GLTSEDSAVYSCARKTISSVVDFYFDYWGQGTTVT |
| SSJ25-C1 | 792 | ELVLTQSPKFMSTSVGDRVSVTCKASQNVGTNVAWYQQKPG |
| VL | | QSPKPLIYSATYRNSGVPDRFTGSGSGTDFTLTITNVQSKDLAD |
| sequence | | YFYFCQYNRYPYTSGGGTKLEIKRRS |

In some embodiments, the antigen binding domain comprises a HC CDR1, a HC CDR2, and a HC CDR3 of any heavy chain binding domain amino acid sequences listed in Table 13A or 14A. In embodiments, the antigen binding domain further comprises a LC CDR1, a LC CDR2, and a LC CDR3. In embodiments, the antigen binding domain comprises a LC CDR1, a LC CDR2, and a LC CDR3 of any light chain binding domain amino acid sequences listed in Table 13A or 14A.

In some embodiments, the antigen binding domain comprises one, two or all of LC CDR1, LC CDR2, and LC CDR3 of any light chain binding domain amino acid sequences listed in Table 13A or 14A, and one, two or all of HC CDR1, HC CDR2, and HC CDR3 of any heavy chain binding domain amino acid sequences listed in Table 13A or 14A.

In some embodiments, the CDRs are defined according to the Kabat numbering scheme, the Chothia numbering scheme, or a combination thereof.

The sequences of humanized CDR sequences of the scFv domains are shown in Table 15A for the heavy chain variable domains and in Table 16A for the light chain variable domains. “ID” stands for the respective SEQ ID NO for each CDR.

TABLE 15A
Heavy Chain Variable Domain CDRs (Kabat)

| Candidate | FW | HCDR1 | ID | HCDR2 | ID | HCDR3 | ID |
|---|---|---|---|---|---|---|---|
| murine_CART19 | | DYGVS | 782 | VIWGSETTYYNSALKS | 783 | HYYYGGSYAMDY | 787 |
| humanized_CART19 a | VH4 | DYGVS | 782 | VIWGSETTYYSSSLKS | 784 | HYYYGGSYAMDY | 787 |
| humanized_CART19 b | VH4 | DYGVS | 782 | VIWGSETTYYQSSLKS | 785 | HYYYGGSYAMDY | 787 |
| humanized_CART19 c | VH4 | DYGVS | 782 | VIWGSETTYYNSSLKS | 786 | HYYYGGSYAMDY | 787 |

TABLE 16A
Light Chain Variable Domain CDRs (Kabat)

| Candidate | FW | LCDR1 | ID | LCDR2 | ID | LCDR3 | ID |
|---|---|---|---|---|---|---|---|
| murine_CART19 | | RASQDISKYLN | 788 | HTSRLHS | 789 | QQGNTLPYT | 790 |
| humanized_CART19 a | VK3 | RASQDISKYLN | 788 | HTSRLHS | 789 | QQGNTLPYT | 790 |
| humanized_CART19 b | VK3 | RASQDISKYLN | 788 | HTSRLHS | 789 | QQGNTLPYT | 790 |
| humanized_CART19 c | VK3 | RASQDISKYLN | 788 | HTSRLHS | 789 | QQGNTLPYT | 790 |

### CAR Construct Components

In embodiments, the CAR scFv fragments are cloned into lentiviral vectors to create a full length CAR construct in a single coding frame, and using the EF1 alpha promoter for expression (SEQ ID NO: 11).

EF1 alpha promoter
CGTGAGGCTCCGGTGCCCGTCAGTGGGCAGAGCGCACATCGCCCACAGTC
CCCGAGAAGTTGGGGGGAGGGGTCGGCAATTGAACCGGTGCCTAGAGAAG
GTGGCGCGGGGTAAACTGGGAAAGTGATGTCGTGTACTGGCTCCGCCTTT
TTCCCGAGGGTGGGGGAGAACCGTATATAAGTGCAGTAGTCGCCGTGAAC
GTTCTTTTTCGCAACGGGTTTGCCGCCAGAACACAGGTAAGTGCCGTGTG
TGGTTCCCGCGGGCCTGGCCTCTTTACGGGTTATGGCCCTTGCGTGCCTT
GAATTACTTCCACCTGGCTGCAGTACGTGATTCTTGATCCCGAGCTTCGG
GTTGGAAGTGGGTGGGAGAGTTCGAGGCCTTGCGCTTAAGGAGCCCCTTC
GCCTCGTGCTTGAGTTGAGGCCTGGCCTGGGCGCTGGGGCCGCCGCGTGC
GAATCTGGTGGCACCTTCGCGCCTGTCTCGCTGCTTTCGATAAGTCTCTA
GCCATTTAAAATTTTTGATGACCTGCTGCGACGCTTTTTTTCTGGCAAGA
TAGTCTTGTAAATGCGGGCCAAGATCTGCACACTGGTATTTCGGTTTTTG
GGGCCGCGGGCGGCGACGGGGCCCGTGCGTCCCAGCGCACATGTTCGGCG
AGGCGGGGCCTGCGAGCGCGGCCACCGAGAATCGGACGGGGGTAGTCTCA
AGCTGGCCGGCCTGCTCTGGTGCCTGGCCTCGCGCCGCCGTGTATCGCCC
CGCCCTGGGCGGCAAGGCTGGCCCGGTCGGCACCAGTTGCGTGAGCGGAA
AGATGGCCGCTTCCCGGCCCTGCTGCAGGGAGCTCAAAATGGAGGACGCG
GCGCTCGGGAGAGCGGGCGGGTGAGTCACCCACACAAAGGAAAAGGGCCT
TTCCGTCCTCAGCCGTCGCTTCATGTGACTCCACGGAGTACCGGGCGCCG
TCCAGGCACCTCGATTAGTTCTCGAGCTTTTGGAGTACGTCGTCTTTAGG
TTGGGGGGAGGGGTTTTATGCGATGGAGTTTCCCCACACTGAGTGGGTGG
AGACTGAAGTTAGGCCAGCTTGGCACTTGATGTAATTCTCCTTGGAATTT
GCCCTTTTTGAGTTTGGATCTTGGTTCATTCTCAAGCCTCAGACAGTGGT
TCAAAGTTTTTTTCTTCCATTTCAGGTGTCGTGA
Gly/Ser

(SEQ ID NO: 25)

GGGGS
Gly/Ser: This sequence may encompass 1-6
“Gly Gly Gly Gly Ser” repeating units

(SEQ ID NO: 26)

GGGGSGGGGS GGGGSGGGGS GGGGSGGGGS
Gly/Ser

(SEQ ID NO: 27)

GGGGSGGGGS GGGGSGGGGS
Gly/Ser

(SEQ ID NO: 28)

GGGGSGGGGS GGGGS
Gly/Ser

(SEQ ID NO: 29)

GGGS

PolyA: (A)₅₀₀₀ (SEQ ID NO:30)

This sequence may encompass 50-5000 adenines.

PolyA: (T)₁₀₀ (SEQ ID NO:31)

PolyA: (T)₅₀₀₀ (SEQ ID NO:32)

This sequence may encompass 50-5000 thymines.

PolyA: (A)₅₀₀₀ (SEQ ID NO:33)

This sequence may encompass 100-5000 adenines.

PolyA: (A)₄₀₀ (SEQ ID NO:34)

This sequence may encompass 100-400 adenines.

PolyA: (A)₂₀₀₀ (SEQ ID NO:35)

This sequence may encompass 50-2000 adenines.

Gly/Ser (SEQ ID NO: 709): This sequence may
encompass 1-10 “Gly Gly Gly Ser”
repeating units
GGGSGGGSGG GSGGGSGGGS GGGSGGGSGG GSGGGSGGGS
Linker (SEQ ID NO: 794)
GSTSGSGKPGSGEGSTKG

The CAR construct can include a Gly/Ser linker having one or more of the following sequences: GGGGS (SEQ ID NO:25); encompassing 1-6 “Gly Gly Gly Gly Ser” repeating units, e.g., GGGGSGGGGS GGGGSGGGGS GGGGSGGGGS (SEQ ID NO:26); GGGGSGGGGS GGGGSGGGGS (SEQ ID NO:27); GGGGSGGGGS GGGGS (SEQ ID NO:28); GGGS (SEQ ID NO:29); or encompassing 1-10 “Gly Gly Gly Ser” repeating units, e.g., GGGSGGGSGG GSGGGSGGGS GGGSGGGSGG GSGGGSGGGS (SEQ ID NO:709).

In embodiments, the CAR construct include a poly A sequence, e.g., a sequence encompassing 50-5000 or 100-5000 adenines (e.g., SEQ ID NO:30, SEQ ID NO:33, SEQ ID NO:34 or SEQ ID NO:35), or a sequence encompassing 50-5000 thymines (e.g., SEQ ID NO:31, SEQ ID NO:32). Alternatively, the CAR construct can include, for example, a linker including the sequence GSTSGSGKPGSGEGSTKG (SEQ ID NO: 704)

Additional sequences/components of a CAR construct can include one or more of the following:

Leader (amino acid sequence)
(SEQ ID NO: 1)
MALPVTALLLPLALLLHAARP
Leader (nucleic acid sequence)
(SEQ ID NO: 12)
ATGGCCCTGCCTGTGACAGCCCTGCTGCTGCCTCTGGCTCTGCTGCTGCATGCC
GCTAGACCC
Leader (codon optimized nucleic acid sequence)
(SEQ ID NO: 796)
ATGGCCCTCCCTGTCACCGCCCTGCTGCTTCCGCTGGCTCTTCTGCTCCACGCCG
CTCGGCCC
CD8 hinge (amino acid sequence)
(SEQ ID NO: 2)
TTTPAPRPPTPAPTIASQPLSLRPEACRPAAGGAVHTRGLDFACD
CD8 hinge (nucleic acid sequence)
(SEQ ID NO: 13)
ACCACGACGCCAGCGCCGCGACCACCAACACCGGCGCCCACCATCGCGTCGCA
GCCCCTGTCCCTGCGCCCAGAGGCGTGCCGGCCAGCGGCGGGGGGCGCAGTGC
ACACGAGGGGGCTGGACTTCGCCTGTGAT
CD8 transmembrane (amino acid sequence)
(SEQ ID NO: 6)
IYIWAPLAGTCGVLLLSLVITLYC
CD8 transmembrane (nucleic acid sequence)
(SEQ ID NO: 17)
ATCTACATCTGGGCGCCCTTGGCCGGGACTTGTGGGGTCCTTCTCCTGTCACTG
GTTATCACCCTTTACTGC
CD8 transmembrane (codon optimized nucleic
acid sequence)
(SEQ ID NO: 797)
ATCTACATTTGGGCCCCTCTGGCTGGTACTTGCGGGGTCCTGCTGCTTTCACTCG
TGATCACTCTTTACTGT
4-1BB Intracellular domain (amino acid sequence)
(SEQ ID NO: 7)
KRGRKKLLYIFKQPFMRPVQTTQEEDGCSCRFPEEEEGGCEL
4-1BB Intracellular domain (nucleic acid sequence)
(SEQ ID NO: 18)
AAACGGGGCAGAAAGAAACTCCTGTATATATTCAAACAACCATTTATGAGACC
AGTACAAACTACTCAAGAGGAAGATGGCTGTAGCTGCCGATTTCCAGAAGAAG
AAGAAGGAGGATGTGAACTG
4-1BB Intracellular domain (codon optimized nucleic
acid sequence)
(SEQ ID NO: 798)
AAGCGCGGTCGGAAGAAGCTGCTGTACATCTTTAAGCAACCCTTCATGAGGCCT
GTGCAGACTACTCAAGAGGAGGACGGCTGTTCATGCCGGTTCCCAGAGGAGGA
GGAAGGCGGCTGCGAACTG
CD28 Intracellular domain (amino acid sequence) (SEQ ID NO: 43)
(SEQ ID NO: 43)
RSKRSRLLHSDYMNMTPRRPGPTRKHYQPYAPPRDFAAYRS
CD28 Intracellular domain (nucleotide sequence)
(SEQ ID NO: 44)
AGGAGTAAGAGGAGCAGGCTCCTGCACAGTGACTACATGAACATGACTCCCCG
CCGCCCCGGGCCCACCCGCAAGCATTACCAGCCCTATGCCCCACCACGCGACTT
CGCAGCCTATCGCTCC (SEQ ID NO: 44)
ICOS Intracellular domain (amino acid sequence)
(SEQ ID NO: 45)
T K K K Y S S S V H D P N G E Y M F M R A V N T A K K
S R L T D V T L
ICOS Intracellular domain (nucleotide sequence) (SEQ ID NO: 46)
(SEQ ID NO: 46)
ACAAAAAAGAAGTATTCATCCAGTGTGCACGACCCTAACGGTGAATACATGTT
CATGAGAGCAGTGAACACAGCCAAAAAATCCAGACTCACAGATGTGACCCTA
(SEQ ID NO: 45)
CD3 zeta domain (Q/K mutant) (amino acid sequence)
(SEQ ID NO: 9)
RVKFSRSADAPAYKQGQNQLYNELNLGRREEYDVLDKRRGRDPEMGGKPRRKNP
QEGLYNELQKDKMAEAYSEIGMKGERRRGKGHDGLYQGLSTATKDTYDALHMQ
ALPPR
CD3 zeta (Q/K mutant) (nucleic acid sequence)
(SEQ ID NO: 20)
AGAGTGAAGTTCAGCAGGAGCGCAGACGCCCCCGCGTACAAGCAGGGCCAGA
ACCAGCTCTATAACGAGCTCAATCTAGGACGAAGAGAGGAGTACGATGTTTTG
GACAAGAGACGTGGCCGGGACCCTGAGATGGGGGGAAAGCCGAGAAGGAAGA
ACCCTCAGGAAGGCCTGTACAATGAACTGCAGAAAGATAAGATGGCGGAGGCC
TACAGTGAGATTGGGATGAAAGGCGAGCGCCGGAGGGGCAAGGGGCACGATG
GCCTTTACCAGGGTCTCAGTACAGCCACCAAGGACACCTACGACGCCCTTCACA
TGCAGGCCCTGCCCCCTCGC
CD3 zeta (Q/K mutant) (codon optimized nucleic
acid sequence)
(SEQ ID NO: 799)
CGCGTGAAATTCAGCCGCAGCGCAGATGCTCCAGCCTACAAGCAGGGGCAGAA
CCAGCTCTACAACGAACTCAATCTTGGTCGGAGAGAGGAGTACGACGTGCTGG
ACAAGCGGAGAGGACGGGACCCAGAAATGGGCGGGAAGCCGCGCAGAAAGAA
TCCCCAAGAGGGCCTGTACAACGAGCTCCAAAAGGATAAGATGGCAGAAGCCT
ATAGCGAGATTGGTATGAAAGGGGAACGCAGAAGAGGCAAAGGCCACGACGG
ACTGTACCAGGGACTCAGCACCGCCACCAAGGACACCTATGACGCTCTTCACAT
GCAGGCCCTGCCGCCTCGG
CD3 zeta domain (amino acid sequence; NCBI Reference
Sequence NM_000734.3)
(SEQ ID NO: 10)
RVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQ
ALPPR
CD3 zeta (nucleic acid sequence; NCBI Reference Sequence
NM_000734.3);
(SEQ ID NO: 21)
AGAGTGAAGTTCAGCAGGAGCGCAGACGCCCCCGCGTACCAGCAGGGCCAG
AACCAGCTCTATAACGAGCTCAATCTAGGACGAAGAGAGGAGTACGATGTTT
TGGACAAGAGACGTGGCCGGGACCCTGAGATGGGGGGAAAGCCGAGAAGGA
AGAACCCTCAGGAAGGCCTGTACAATGAACTGCAGAAAGATAAGATGGCGG
AGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGC
CCTTCACATGCAGGCCCTGCCCCCTCGC
IgG4 Hinge (amino acid sequence)
(SEQ ID NO: 3)
ESKYGPPCPPCPAPEFLGGPSVFLFPPKPKDTLMISRTPEVTCVVVDVSQEDPEVQFN
WYVDGVEVHNAKTKPREEQFNSTYRVVSVLTVLHQDWLNGKEYKCKVSNKGLPS
SIEKTISKAKGQPREPQVYTLPPSQEEMTKNQVSLTCLVKGFYPSDIAVEWESNGQP
ENNYKTTPPVLDSDGSFFLYSRLTVDKSRWQEGNVFSCSVMHEALHNHYTQKSLS
LSLGKM
IgG4 Hinge (nucleotide sequence)
(SEQ ID NO: 14)
GAGAGCAAGTACGGCCCTCCCTGCCCCCCTTGCCCTGCCCCCGAGTTCCTGGGC
GGACCCAGCGTGTTCCTGTTCCCCCCCAAGCCCAAGGACACCCTGATGATCAGC
CGGACCCCCGAGGTGACCTGTGTGGTGGTGGACGTGTCCCAGGAGGACCCCGA
GGTCCAGTTCAACTGGTACGTGGACGGCGTGGAGGTGCACAACGCCAAGACCA
AGCCCCGGGAGGAGCAGTTCAATAGCACCTACCGGGTGGTGTCCGTGCTGACC
GTGCTGCACCAGGACTGGCTGAACGGCAAGGAATACAAGTGTAAGGTGTCCAA
CAAGGGCCTGCCCAGCAGCATCGAGAAAACCATCAGCAAGGCCAAGGGCCAG
CCTCGGGAGCCCCAGGTGTACACCCTGCCCCCTAGCCAAGAGGAGATGACCAA
GAACCAGGTGTCCCTGACCTGCCTGGTGAAGGGCTTCTACCCCAGCGACATCGC
CGTGGAGTGGGAGAGCAACGGCCAGCCCGAGAACAACTACAAGACCACCCCCC
CTGTGCTGGACAGCGACGGCAGCTTCTTCCTGTACAGCCGGCTGACCGTGGACA
AGAGCCGGTGGCAGGAGGGCAACGTCTTTAGCTGCTCCGTGATGCACGAGGCC
CTGCACAACCACTACACCCAGAAGAGCCTGAGCCTGTCCCTGGGCAAGATG
IgD hinge (aa)
(SEQ ID NO: 4)
RWPESPKAQASSVPTAQPQAEGSLAKATTAPATTRNTGRGGEEKKKEKEKEEQEERET
KTPECPSHTQPLGVYLLTPAVQDLWLRDKATFTCFVVGSDLKDAHLTWEVAGKVPTG
GVEEGLLERHSNGSQSQHSRLTLPRSLWNAGTSVTCTLNHPSLPPQRLMALREPAAQA
PVKLSLNLLASSDPPEAASWLLCEVSGFSPPNILLMWLEDQREVNTSGFAPARPPPQPG
STTFWAWSVLRVPAPPSPQPATYTCVVSHEDSRTLLNASRSLEVSYVTDH
IgD hinge (na)
(SEQ ID NO: 15)
AGGTGGCCCGAAAGTCCCAAGGCCCAGGCATCTAGTGTTCCTACTGCACAGCCCCA
GGCAGAAGGCAGCCTAGCCAAAGCTACTACTGCACCTGCCACTACGCGCAATACT
GGCCGTGGCGGGGAGGAGAAGAAAAAGGAGAAAGAGAAAGAAGAACAGGAAGA
GAGGGAGACCAAGACCCCTGAATGTCCATCCCATACCCAGCCGCTGGGCGTCTATC
TCTTGACTCCCGCAGTACAGGACTTGTGGCTTAGAGATAAGGCCACCTTTACATGT
TTCGTCGTGGGCTCTGACCTGAAGGATGCCCATTTGACTTGGGAGGTTGCCGGAAA
GGTACCCACAGGGGGGGTTGAGGAAGGGTTGCTGGAGCGCCATTCCAATGGCTCT
CAGAGCCAGCACTCAAGACTCACCCTTCCGAGATCCCTGTGGAACGCCGGGACCTC
TGTCACATGTACTCTAAATCATCCTAGCCTGCCCCCACAGCGTCTGATGGCCCTTAG
AGAGCCAGCCGCCCAGGCACCAGTTAAGCTTAGCCTGAATCTGCTCGCCAGTAGTG
ATCCCCCAGAGGCCGCCAGCTGGCTCTTATGCGAAGTGTCCGGCTTTAGCCCGCCC
AACATCTTGCTCATGTGGCTGGAGGACCAGCGAGAAGTGAACACCAGCGGCTTCG
CTCCAGCCCGGCCCCCACCCCAGCCGGGTTCTACCACATTCTGGGCCTGGAGTGTC
TTAAGGGTCCCAGCACCACCTAGCCCCCAGCCAGCCACATACACCTGTGTTGTGTC
CCATGAAGATAGCAGGACCCTGCTAAATGCTTCTAGGAGTCTGGAGGTTTCCTACG
TGACTGACCATT
CD27 (aa)
(SEQ ID NO: 8)
QRRKYRSNKGESPVEPAEPCRYSCPREEEGSTIPIQEDYRKPEPACSP
CD27 (na)
(SEQ ID NO: 19)
AGGAGTAAGAGGAGCAGGCTCCTGCACAGTGACTACATGAACATGACTCCCCGCC
GCCCCGGGCCCACCCGCAAGCATTACCAGCCCTATGCCCCACCACGCGACTTCGCA
GCCTATCGCTCC
Y to F mutant ICOS domain (aa)
(SEQ ID NO: 795)
TKKKYSSSVHDPNGEFMFMRAVNTAKKSRLTDVTL

### Bispecific CARs

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH-CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also encompassed creating for bispecifc, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus futher associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁-VH₁-VH₂-VL₂. Optionally, a linker is disposed between the two antibodies or antibody fragments (e.g., scFvs), e.g., between VL₁ and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser)n linker, wherein n is 1, 2, 3, 4, 5, or 6, preferably 4 (SEQ ID NO: 26). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

In one aspect, the bispecific antibody molecule is characterized by a first immunoglobulin variable domain sequence, e.g., a scFv, which has binding specificity for an antigen (e.g., tumor antigen, e.g., B cell antigen, e.g., CD123 or CD19), e.g., comprises a scFv as described herein, e.g., as described in Table 11A, Table 12A, Table 12B, Table 13A, or Table 14A, or comprises the light chain CDRs and/or heavy chain CDRs from a scFv (e.g., CD123 or CD19 scFv) described herein, and a second immunoglobulin variable domain sequence that has binding specificity for a second epitope on a different antigen. In some aspects the second immunoglobulin variable domain sequence has binding specificity for an antigen expressed on AML cells, e.g., an antigen other than CD123. For example, the second immunoglobulin variable domain sequence has binding specificity for CLL-1. As another example, the second immunoglobulin variable domain sequence has binding specificity for CD33. As another example, the second immunoglobulin variable domain sequence has binding specificity for CD34. As another example, the second immunoglobulin variable domain sequence has binding specificity for FLT3. For example, the second immunoglobulin variable domain sequence has binding specificity for folate receptor beta. In some aspects, the second immunoglobulin variable domain sequence has binding specificity for an antigen expressed on B-cells, for example, CD19, CD20, CD22 or ROR1.

### Chimeric TCR

In one aspect, the antibodies and antibody fragments (e.g., anti-CD123 antibodies or antibody fragments) of the present invention (for example, those disclosed in Tables 11A, 12A, 12B, 13A, or 14A) can be grafted to one or more constant domain of a T cell receptor (“TCR”) chain, for example, a TCR alpha or TCR beta chain, to create an chimeric TCR that binds specificity to the antigen (e.g., tumor antigen, e.g., B cell antigen, e.g, CD123 or CD19). Without being bound by theory, it is believed that chimeric TCRs will signal through the TCR complex upon antigen binding. For example, a scFv (e.g., CD123 scFv or CD19 scFv) as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment (e.g., anti-CD123 antibody fragment or anti-CD19 antibody fragment), for example a VL domain as described herein, can be grafted to the constant domain of a TCR alpha chain, and an antibody fragment (e.g., anti-CD123 antibody fragment or anti-CD19 antibody fragment), for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment (e.g., CD123 antibody or antibody fragment, e.g., the CDRs of a CD123 antibody or antibody fragment as described in Tables 1A, 2A, 3A, 4A, 5A, 6A, 7A, 8A, 10A, or 12A; or the CDRs of a CD19 antibody or antibody fragment, e.g., described in Tables 13A, 14A, 15A, or 16A) may be grafted into a TCR alpha and/or beta chain to create a chimeric TCR that binds specifically to the antigen (e.g., CD123 or CD19). For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced by methods known in the art (for example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

### Stability and Mutations

The stability of an antigen binding domain (e.g., tumor antigen binding domain, e.g., B cell antigen binding domain, e.g., CD123 binding domain or CD19 binding domain), e.g., scFv molecules (e.g., soluble scFv) can be evaluated in reference to the biophysical properties (e.g., thermal stability, percent aggregation, and binding affinity) of, e.g., a conventional control scFv molecule or a full length antibody as described on pages 147-151 of WO 2016/028896 filed on Aug. 19, 2015, the entire contents of which are hereby incorporated by reference.

In one aspect, the antigen binding domain of the CAR comprises an amino acid sequence that is homologous to an antigen binding domain amino acid sequence described herein, and the antigen binding domain retains the desired functional properties of the CD123 antibody fragments described herein. In one specific aspect, the CAR composition of the invention comprises an antibody fragment. In a further aspect, that antibody fragment comprises an scFv.

In various aspects, the antigen binding domain of the CAR is engineered by modifying one or more amino acids within one or both variable regions (e.g., VH and/or VL), for example within one or more CDR regions and/or within one or more framework regions. In one specific aspect, the CAR composition of the invention comprises an antibody fragment. In a further aspect, that antibody fragment comprises an scFv.

It will be understood by one of ordinary skill in the art that the antibody or antibody fragment of the invention may further be modified such that they vary in amino acid sequence (e.g., from wild-type), but not in desired activity. For example, additional nucleotide substitutions leading to amino acid substitutions at “non-essential” amino acid residues may be made to the protein For example, a nonessential amino acid residue in a molecule may be replaced with another amino acid residue from the same side chain family. In another embodiment, a string of amino acids can be replaced with a structurally similar string that differs in order and/or composition of side chain family members, e.g., a conservative substitution, in which an amino acid residue is replaced with an amino acid residue having a similar side chain, may be made.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Percent identity in the context of two or more nucleic acids or polypeptide sequences, refers to two or more sequences that are the same. Two sequences are “substantially identical” if two sequences have a specified percentage of amino acid residues or nucleotides that are the same (e.g., 60% identity, optionally 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be conducted, e.g., by the local homology algorithm of Smith and Waterman, (1970) Adv. Appl. Math. 2:482c, by the homology alignment algorithm of Needleman and Wunsch, (1970) J. Mol. Biol. 48:443, by the search for similarity method of Pearson and Lipman, (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., (2003) Current Protocols in Molecular Biology).

Two examples of algorithms that are suitable for determining percent sequence identity and sequence similarity are the BLAST and BLAST 2.0 algorithms, which are described in Altschul et al., (1977) Nuc. Acids Res. 25:3389-3402; and Altschul et al., (1990) J. Mol. Biol. 215:403-410, respectively. Software for performing BLAST analyses is publicly available through the National Center for Biotechnology Information.

The percent identity between two amino acid sequences can also be determined using the algorithm of E. Meyers and W. Miller, (1988) Comput. Appl. Biosci. 4:11-17) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (1970) J. Mol. Biol. 48:444-453) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

In one aspect, the present invention contemplates modifications of the starting antibody or fragment (e.g., scFv) amino acid sequence that generate functionally equivalent molecules. For example, the VH or VL of an antigen binding domain (e.g., tumor antigen binding domain, e.g., B cell antigen binding domain, e.g., CD123 binding domain or CD19 binding domain), e.g., scFv, comprised in the CAR can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting VH or VL framework region of the antigen binding domain (e.g., tumor antigen binding domain, e.g., B cell antigen binding domain, e.g., CD123 binding domain or CD19 binding domain), e.g., scFv. The present invention contemplates modifications of the entire CAR construct, e.g., modifications in one or more amino acid sequences of the various domains of the CAR construct in order to generate functionally equivalent molecules. The CAR construct can be modified to retain at least about 70%, 71%. 72%. 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identity of the starting CAR construct.

### Antigens

In accordance with any method or composition described herein, exemplary tumor antigens include but are not limited to one or more of the following: thyroid stimulating hormone receptor (TSHR); CD171; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); epidermal growth factor receptor (EGFR); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer; TGS5; high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In embodiments, the tumor antigen is selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6,E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.

In embodiments, the tumor antigen is a B cell antigen (e.g., B cell surface antigen), e.g., CD10, CD19, CD20, CD22, CD34, CD123, FLT-3, ROR1, CD79b, CD179b, or CD79a.

In embodiments, the tumor antigen is CD123. In embodiments, the tumor antigen is CD19. In other embodiments, the tumor antigen is BCMA, CLL-1, or EGFRvIII.

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the otherdomains of the CAR is used. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the CAR-expressing cell, e.g., CART cell, cell surface. In a different aspect the amino acid sequence of the transmembrane domain may be modified or substituted so as to minimize interactions with the binding domains of the native binding partner present in the same CAR-expressing cell, e.g., CART cell.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, and CD19.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge. In one embodiment, the hinge or spacer comprises (e.g., consists of) the amino acid sequence of SEQ ID NO:2. In one aspect, the transmembrane domain comprises (e.g., consists of) a transmembrane domain of SEQ ID NO: 6.

In one aspect, the hinge or spacer comprises an IgG4 hinge. For example, in one embodiment, the hinge or spacer comprises a hinge of the amino acid sequence ESKYGPPCPPCPAPEFLGGPSVFLFPPKPKDTLMISRTPEVTCVVVDVSQEDPEVQFNW YVDGVEVHNAKTKPREEQFNSTYRVVSVLTVLHQDWLNGKEYKCKVSNKGLPSSIEK TISKAKGQPREPQVYTLPPSQEEMTKNQVSLTCLVKGFYPSDIAVEWESNGQPENNYK TTPPVLDSDGSFFLYSRLTVDKSRWQEGNVFSCSVMHEALHNHYTQKSLSLSLGKM (SEQ ID NO:3). In some embodiments, the hinge or spacer comprises a hinge encoded by a nucleotide sequence of

(SEQ ID NO: 14)

GAGAGCAAGTACGGCCCTCCCTGCCCCCCTTGCCCTGCCCCCGAGTTCCT
GGGCGGACCCAGCGTGTTCCTGTTCCCCCCCAAGCCCAAGGACACCCTGA
TGATCAGCCGGACCCCCGAGGTGACCTGTGTGGTGGTGGACGTGTCCCAG
GAGGACCCCGAGGTCCAGTTCAACTGGTACGTGGACGGCGTGGAGGTGCA
CAACGCCAAGACCAAGCCCCGGGAGGAGCAGTTCAATAGCACCTACCGGG
TGGTGTCCGTGCTGACCGTGCTGCACCAGGACTGGCTGAACGGCAAGGAA
TACAAGTGTAAGGTGTCCAACAAGGGCCTGCCCAGCAGCATCGAGAAAAC
CATCAGCAAGGCCAAGGGCCAGCCTCGGGAGCCCCAGGTGTACACCCTGC
CCCCTAGCCAAGAGGAGATGACCAAGAACCAGGTGTCCCTGACCTGCCTG
GTGAAGGGCTTCTACCCCAGCGACATCGCCGTGGAGTGGGAGAGCAACGG
CCAGCCCGAGAACAACTACAAGACCACCCCCCCTGTGCTGGACAGCGACG
GCAGCTTCTTCCTGTACAGCCGGCTGACCGTGGACAAGAGCCGGTGGCAG
GAGGGCAACGTCTTTAGCTGCTCCGTGATGCACGAGGCCCTGCACAACCA
CTACACCCAGAAGAGCCTGAGCCTGTCCCTGGGCAAGATG.

In one aspect, the hinge or spacer comprises an IgD hinge. For example, in one embodiment, the hinge or spacer comprises a hinge of the amino acid sequence RWPESPKAQASSVPTAQPQAEGSLAKATTAPATTRNTGRGGEEKKKEKEKEEQEERET KTPECPSHTQPLGVYLLTPAVQDLWLRDKATFTCFVVGSDLKDAHLTWEVAGKVPTG GVEEGLLERHSNGSQSQHSRLTLPRSLWNAGTSVTCTLNHPSLPPQRLMALREPAAQA PVKLSLNLLASSDPPEAASWLLCEVSGFSPPNILLMWLEDQREVNTSGFAPARPPPQPG STTFWAWSVLRVPAPPSPQPATYTCVVSHEDSRTLLNASRSLEVSYVTDH (SEQ ID NO:4). In some embodiments, the hinge or spacer comprises a hinge encoded by a nucleotide sequence of

(SEQ ID NO: 15)

AGGTGGCCCGAAAGTCCCAAGGCCCAGGCATCTAGTGTTCCTACTGCAC
AGCCCCAGGCAGAAGGCAGCCTAGCCAAAGCTACTACTGCACCTGCCAC
TACGCGCAATACTGGCCGTGGCGGGGAGGAGAAGAAAAAGGAGAAAGAG
AAAGAAGAACAGGAAGAGAGGGAGACCAAGACCCCTGAATGTCCATCCC
ATACCCAGCCGCTGGGCGTCTATCTCTTGACTCCCGCAGTACAGGACTT
GTGGCTTAGAGATAAGGCCACCTTTACATGTTTCGTCGTGGGCTCTGAC
CTGAAGGATGCCCATTTGACTTGGGAGGTTGCCGGAAAGGTACCCACAG
GGGGGGTTGAGGAAGGGTTGCTGGAGCGCCATTCCAATGGCTCTCAGAG
CCAGCACTCAAGACTCACCCTTCCGAGATCCCTGTGGAACGCCGGGACC
TCTGTCACATGTACTCTAAATCATCCTAGCCTGCCCCCACAGCGTCTGA
TGGCCCTTAGAGAGCCAGCCGCCCAGGCACCAGTTAAGCTTAGCCTGAA
TCTGCTCGCCAGTAGTGATCCCCCAGAGGCCGCCAGCTGGCTCTTATGC
GAAGTGTCCGGCTTTAGCCCGCCCAACATCTTGCTCATGTGGCTGGAGG
ACCAGCGAGAAGTGAACACCAGCGGCTTCGCTCCAGCCCGGCCCCCACC
CCAGCCGGGTTCTACCACATTCTGGGCCTGGAGTGTCTTAAGGGTCCCA
GCACCACCTAGCCCCCAGCCAGCCACATACACCTGTGTTGTGTCCCATG
AAGATAGCAGGACCCTGCTAAATGCTTCTAGGAGTCTGGAGGTTTCCTA
CGTGACTGACCATT.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO:5). In some embodiments, the linker is encoded by a nucleotide sequence of

(SEQ ID NO: 16)

GGTGGCGGAGGTTCTGGAGGTGGAGGTTCC.

In one aspect, the hinge or spacer comprises a KIR2DS2 hinge.

### Cytoplasmic Domain

The cytoplasmic domain or region of the present CAR includes an intracellular signaling domain. An intracellular signaling domain is capable of activation of at least one of the normal effector functions of the immune cell in which the CAR has been introduced.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In one embodiment, a CAR of the invention comprises an intracellular signaling domain, e.g., a primary signaling domain of CD3-zeta.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

Further examples of molecules containing a primary intracellular signaling domain that are of particular use in the invention include those of DAP10, DAP12, and CD32.

The intracellular signalling domain of the CAR can comprise the primary signalling domain, e.g., CD3-zeta signaling domain, by itself or it can be combined with any other desired intracellular signaling domain(s) useful in the context of a CAR of the invention. For example, the intracellular signaling domain of the CAR can comprise a primary signalling domain, e.g., CD3 zeta chain portion, and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include a MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. For example, CD27 costimulation has been demonstrated to enhance expansion, effector function, and survival of human CART cells in vitro and augments human T cell persistence and antitumor activity in vivo (Song et al. Blood. 2012; 119(3):696-706).

The intracellular signaling sequences within the cytoplasmic portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 7. In one aspect, the signaling domain of CD3-zeta is a signaling domain of SEQ ID NO: 9 (mutant CD3-zeta) or SEQ ID NO: 10 (wild type human CD3-zeta).

In one aspect, the intracellular signaling domain is designed to comprise the signaling domain of CD3-zeta and the signaling domain of CD27. In one aspect, the signaling domain of CD27 comprises an amino acid sequence of QRRKYRSNKGESPVEPAEPCRYSCPREEEGSTIPIQEDYRKPEPACSP (SEQ ID NO:8). In one aspect, the signalling domain of CD27 is encoded by a nucleic acid sequence of

(SEQ ID NO: 19)

AGGAGTAAGAGGAGCAGGCTCCTGCACAGTGACTACATGAACATGACTCC
CCGCCGCCCCGGGCCCACCCGCAAGCATTACCAGCCCTATGCCCCACC
ACGCGACTTCGCAGCCTATCGCTCC.

In one aspect, the intracellular is designed to comprise the signaling domain of CD3-zeta and the signaling domain of CD28. In one aspect, the signaling domain of CD28 comprises an amino acid sequence of SEQ ID NO: 43. In one aspect, the signaling domain of CD28 is encoded by a nucleic acid sequence of SEQ ID NO: 44.

In one aspect, the intracellular is designed to comprise the signaling domain of CD3-zeta and the signaling domain of ICOS. In one aspect, the signaling domain of ICOS comprises an amino acid sequence of SEQ ID NO: 45. In one aspect, the signaling domain of ICOS is encoded by a nucleic acid sequence of SEQ ID NO: 46.

In one aspect, the CAR-expressing cell described herein can further comprise a second CAR, e.g., a second CAR that includes a different antigen binding domain, e.g., to the same target (e.g., CD123 or CD19, or any other antigen described herein) or a different target (e.g., CD19, CD33, CLL-1, CD34, FLT3, or folate receptor beta, or any other antigen described herein). In one embodiment, the second CAR includes an antigen binding domain to a target expressed on acute myeloid leukemia cells, such as, e.g., CD19, CD33, CLL-1, CD34, FLT3, or folate receptor beta. In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, CD27, ICOS or OX-40, onto the first CAR, and the primary signaling domain, e.g., CD3 zeta, on the second CAR can limit the CAR activity to cells where both targets are expressed. In one embodiment, the CAR expressing cell comprises a first CD123 CAR that includes a CD123 binding domain, a transmembrane domain and a costimulatory domain and a second CAR that targets an antigen other than CD123 (e.g., an antigen expressed on AML cells, e.g., CD19, CD33, CLL-1, CD34, FLT3, or folate receptor beta) and includes an antigen binding domain, a transmembrane domain and a primary signaling domain. In another embodiment, the CAR expressing cell comprises a first CD123 CAR that includes a CD123 binding domain, a transmembrane domain and a primary signaling domain and a second CAR that targets an antigen other than CD123 (e.g., an antigen expressed on AML cells, e.g., CD19, CD33, CLL-1, CD34, FLT3, or folate receptor beta) and includes an antigen binding domain to the antigen, a transmembrane domain and a costimulatory signaling domain.

In one embodiment, the CAR-expressing cell comprises a CAR described herein (e.g., CD123 CAR or CD19 CAR described herein) and an inhibitory CAR. In one embodiment, the inhibitory CAR comprises an antigen binding domain that binds an antigen found on normal cells but not cancer cells, e.g., normal cells that also express CD123 or CD19. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta).

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (e.g., selected by phage display).

It has also been discovered, that cells having a plurality of chimeric membrane embedded receptors comprising an antigen binding domain that interactions between the antigen binding domain of the receptors can be undesirable, e.g., because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising a antigen binding domains that minimize such interactions, as well as methods of making and using such cells and nucleic acids. In an embodiment the antigen binding domain of one of said first said second non-naturally occurring chimeric membrane embedded receptor, comprises an scFv, and the other comprises a single VH domain, e.g., a camelid, shark, or lamprey single VH domain, or a single VH domain derived from a human or mouse sequence.

In some embodiments, the claimed invention comprises a first and second CAR, wherein the antigen binding domain of one of said first CAR said second CAR does not comprise a variable light domain and a variable heavy domain. In some embodiments, the antigen binding domain of one of said first CAR said second CAR is an scFv, and the other is not an scFv. In some embodiments, the antigen binding domain of one of said first CAR said second CAR comprises a single VH domain, e.g., a camelid, shark, or lamprey single VH domain, or a single VH domain derived from a human or mouse sequence. In some embodiments, the antigen binding domain of one of said first CAR said second CAR comprises a nanobody. In some embodiments, the antigen binding domain of one of said first CAR said second CAR comprises a camelid VHH domain.

In some embodiments, the antigen binding domain of one of said first CAR said second CAR comprises an scFv, and the other comprises a single VH domain, e.g., a camelid, shark, or lamprey single VH domain, or a single VH domain derived from a human or mouse sequence. In some embodiments, the antigen binding domain of one of said first CAR said second CAR comprises an scFv, and the other comprises a nanobody. In some embodiments, the antigen binding domain of one of said first CAR said second CAR comprises comprises an scFv, and the other comprises a camelid VHH domain.

In some embodiments, when present on the surface of a cell, binding of the antigen binding domain of said first CAR to its cognate antigen is not substantially reduced by the presence of said second CAR. In some embodiments, binding of the antigen binding domain of said first CAR to its cognate antigen in the presence of said second CAR is 85%, 90%, 95%, 96%, 97%, 98% or 99% of binding of the antigen binding domain of said first CAR to its cognate antigen in the absence of said second CAR.

In some embodiments, when present on the surface of a cell, the antigen binding domains of said first CAR said second CAR, associate with one another less than if both were scFv antigen binding domains. In some embodiments, the antigen binding domains of said first CAR said second CAR, associate with one another 85%, 90%, 95%, 96%, 97%, 98% or 99% less than if both were scFv antigen binding domains.

In another aspect, the CAR-expressing cell described herein can further express another agent, e.g., an agent which enhances the activity of a CAR-expressing cell. For example, in one embodiment, the agent can be an agent which inhibits an inhibitory molecule. Inhibitory molecules, e.g., PD1, can, in some embodiments, decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta). In one embodiment, the agent which inhibits an inhibitory molecule, e.g., is a molecule described herein, e.g., an agent that comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta), or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). In embodiments, the CAR-expressing cell described herein comprises a switch costimulatory receptor, e.g., as described in WO 2013/019615, which is incorporated herein by reference in its entirety. PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1), can be fused to a transmembrane domain and intracellular signaling domains such as 41BB and CD3 zeta (also referred to herein as a PD1 CAR). In one embodiment, the PD1 CAR, when used incombinations with a CD123 CAR described herein, improves the persistence of the CAR-expressing cell, e.g., T cell or NK cell. In one embodiment, the CAR is a PD1 CAR comprising the extracellular domain of PD1 indicated as underlined in SEQ ID NO: 24. In one embodiment, the PD1 CAR comprises the amino acid sequence of SEQ ID NO:24.

(SEQ ID NO: 24)

Malpvtalllplalllhaarppgwfldspdrpwnpptfspallvvtegdn
atftcsfsntsesfvlnwyrmspsnqtdklaafpedrsqpgqdcrfrvtq
lpngrdfhmsvvrarrndsgtylcgaislapkaqikeslraelrvterra
evptahpspsprpagqfqtlvtttpaprpptpaptiasqplslrpeacrp
aaggavhtrgldfacdiyiwaplagtcgvlllslvitlyckrgrkkllyi
fkqpfmrpvqttqeedgcscrfpeeeeggcelrvkfsrsadapaykqgqn
qlynelnlgrreeydvldkrrgrdpemggkprrknpqeglynelqkdkma
eayseigmkgerrrgkghdglyqglstatkdtydalhmqalppr.

In one embodiment, the PD1 CAR comprises the amino acid sequence provided below (SEQ ID NO:22).

(SEQ ID NO: 22)

pgwfldspdrpwnpptfspallvvtegdnatftcsfsntsesfvlnwyrm
spsnqtdklaafpedrsqpgqdcrfrvtqlpngrdfhmsvvrarrndsgt
ylcgaislapkaqikeslraelrvterraevptahpspsprpagqfqtlv
tttpaprpptpaptiasqplslrpeacrpaaggavhtrgldfacdiyiwa
plagtcgvlllslvitlyckrgrkkllyifkqpfmrpvqttqeedgcscr
fpeeeeggcelrykfsrsadapaykqgqnqlynelnlgrreeydvldkrr
grdpemggkprrknpqeglynelqkdkmaeayseigmkgerrrgkghdgl
yqglstatkdtydalhmqalppr.

In one embodiment, the agent comprises a nucleic acid sequence encoding the PD1 CAR, e.g., the PD1 CAR described herein. In one embodiment, the nucleic acid sequence for the PD1 CAR is shown below, with the PD1 ECD underlined below in SEQ ID NO: 23

(SEQ ID NO: 23)
atggccctccctgtcactgccctgcttctccccctcgcactcctgctcca
cgccgctagaccacccggatggtttctggactctccggatcgcccgtgga
atcccccaaccttctcaccggcactcttggttgtgactgagggcgataat
gcgaccttcacgtgctcgttctccaacacctccgaatcattcgtgctgaa
ctggtaccgcatgagcccgtcaaaccagaccgacaagctcgccgcgtttc
cggaagatcggtcgcaaccgggacaggattgtcggttccgcgtgactcaa
ctgccgaatggcagagacttccacatgagcgtggtccgcgctaggcgaaa
cgactccgggacctacctgtgcggagccatctcgctggcgcctaaggccc
aaatcaaagagagcttgagggccgaactgagagtgaccgagcgcagagct
gaggtgccaactgcacatccatccccatcgcctcggcctgcggggcagtt
tcagaccctggtcacgaccactccggcgccgcgcccaccgactccggccc
caactatcgcgagccagcccctgtcgctgaggccggaagcatgccgccct
gccgccggaggtgctgtgcatacccggggattggacttcgcatgcgacat
ctacatttgggctcctctcgccggaacttgtggcgtgctccttctgtccc
tggtcatcaccctgtactgcaagcggggtcggaaaaagcttctgtacatt
ttcaagcagcccttcatgaggcccgtgcaaaccacccaggaggaggacgg
ttgctcctgccggttccccgaagaggaagaaggaggttgcgagctgcgcg
tgaagttctcccggagcgccgacgcccccgcctataagcagggccagaac
cagctgtacaacgaactgaacctgggacggcgggaagagtacgatgtgct
ggacaagcggcgcggccgggaccccgaaatgggcgggaagcctagaagaa
agaaccctcaggaaggcctgtataacgagctgcagaaggacaagatggcc
gaggcctactccgaaattgggatgaagggagagcggcggaggggaaaggg
gcacgacggcctgtaccaaggactgtccaccgccaccaaggacacatacg
atgccctgcacatgcaggcccttccccctcgc.

In another aspect, the present invention provides a population of CAR-expressing cells, e.g., CART cells or CAR-expressing NK cells. In some embodiments, the population of CAR-expressing cells comprises a mixture of cells expressing different CARs. For example, in one embodiment, the population of CAR-expressing cells (e.g., CART cells or CAR-expressing NK cells) can include a first cell expressing a CAR having an antigen binding domain (e.g., tumor antigen binding domain, e.g., B cell antigen binding domain, e.g., CD123 binding domain or CD19 binding domain) described herein, and a second cell expressing a CAR having a different antigen binding domain (e.g., tumor antigen binding domain, e.g., B cell antigen binding domain, e.g., CD123 binding domain or CD19 binding domain), e.g., an antigen binding domain described herein that differs from the antigen binding domain in the CAR expressed by the first cell. As another example, the population of CAR-expressing cells can include a first cell expressing a CAR that includes a CD123 binding domain, e.g., as described herein, and a second cell expressing a CAR that includes an antigen binding domain to a target other than CD123 (e.g., CD33, CD34, CLL-1, FLT3, CD19, CD20, CD22, or folate receptor beta). In one embodiment, the population of CAR-expressing cells includes, e.g., a first cell expressing a CAR that includes a primary intracellular signaling domain, and a second cell expressing a CAR that includes a secondary signaling domain, e.g., a costimulatory signaling domain.

In another aspect, the present invention provides a population of cells wherein at least one cell in the population expresses a CAR having antigen binding domain (e.g., tumor antigen binding domain, e.g., B cell antigen binding domain, e.g., CD123 binding domain or CD19 binding domain) described herein, and a second cell expressing another agent, e.g., an agent which enhances the activity of a CAR-expressing cell. For example, in one embodiment, the agent can be an agent which inhibits an inhibitory molecule. Inhibitory molecules, e.g., can, in some embodiments, decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta). In one embodiment, the agent which inhibits an inhibitory molecule, e.g., is a molecule described herein, e.g., an agent that comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (e.g., TGF beta), or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of the extracellular domain of PD1), and a second polypeptide of an intracellular signaling domain described herein (e.g., a CD28 signaling domain described herein and/or a CD3 zeta signaling domain described herein).

In one aspect, the present invention provides methods comprising administering a population of CAR-expressing cells, e.g., CART cells or CAR-expressing NK cells, e.g., a mixture of cells expressing different CARs, in combination with another agent, e.g., a kinase inhibitor, such as a kinase inhibitor described herein. In another aspect, the present invention provides methods comprising administering a population of cells wherein at least one cell in the population expresses a CAR having an anti-cancer associated antigen binding domain as described herein, and a second cell expressing another agent, e.g., an agent which enhances the activity of a CAR-expressing cell, in combination with another agent, e.g., a kinase inhibitor, such as a kinase inhibitor described herein.

### Natural Killer Cell Receptor (NKR) CARs

In an embodiment, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cyotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell uses a split CAR. The split CAR approach is described in more detail in publications WO2014/055442 and WO2014/055657, incorporated herein by reference. Briefly, a split CAR system comprises a cell expressing a first CAR having a first antigen binding domain and a costimulatory domain (e.g., 4-1BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens. In embodiments the first antigen binding domain recognizes an antigen described herein (e.g., a B cell antigen, e.g., CD123 or CD19), e.g., comprises an antigen binding domain described herein, and the second antigen binding domain recognizes an antigen expressed on acute myeloid leukemia cells, e.g., CLL-1, CD33, CD34, FLT3, or folate receptor beta. In embodiments the first antigen binding domain recognizes CD123, e.g., comprises an antigen binding domain described herein, and the second antigen binding domain recognizes an antigen expressed on B-cells, e.g., CD19, CD20, CD22 or ROR1.

### Strategies for Regulating Chimeric Antigen Receptors

There are many ways CAR activities can be regulated. In some embodiments, a regulatable CAR (RCAR) where the CAR activity can be controlled is desirable to optimize the safety and efficacy of a CAR therapy. For example, inducing apoptosis using, e.g., a caspase fused to a dimerization domain (see, e.g., Di et al., N Engl. J. Med. 2011 Nov. 3; 365(18):1673-1683), can be used as a safety switch in the CAR therapy of the instant invention. In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or complement-induced cell death. For example, CAR expressing cells described herein may also express a receptor capable of being targeted by an antibody or antibody fragment. Examples of such receptors include EpCAM, VEGFR, integrins (e.g., integrins ανβ3, α4, αI¾β3, α4β7, α5β1, ανβ3, αν), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD1 1, CD1 1 a/LFA-1, CD15, CD18/ITGB2, CD19, CD20, CD22, CD23/1gE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g, ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In other embodiments, a CAR-expressing cell described herein may also express a target protein recognized by the T cell depleting agent. In one embodiment, the target protein is CD20 and the T cell depleting agent is an anti-CD20 antibody, e.g., rituximab. In such embodiment, the T cell depleting agent is administered once it is desirable to reduce or eliminate the CAR-expressing cell, e.g., to mitigate the CAR induced toxicity. In other embodiments, the T cell depleting agent is an anti-CD52 antibody, e.g., alemtuzumab, as described in the Examples herein.

In other embodiments, a RCAR comprises a set of polypeptides, typically two in the simplest embodiments, in which the components of a standard CAR described herein, e.g., an antigen binding domain and an intracellular signaling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signaling domain. Additional description and exemplary configurations of such regulatable CARs are provided herein and in International Publication No. WO 2015/090229, hereby incorporated by reference in its entirety.

In an aspect, an RCAR comprises two polypeptides or members: 1) an intracellular signaling member comprising an intracellular signaling domain, e.g., a primary intracellular signaling domain described herein, and a first switch domain; 2) an antigen binding member comprising an antigen binding domain, e.g., that specifically binds a tumor antigen described herein, as described herein and a second switch domain. Optionally, the RCAR comprises a transmembrane domain described herein. In an embodiment, a transmembrane domain can be disposed on the intracellular signaling member, on the antigen binding member, or on both. (Unless otherwise indicated, when members or elements of an RCAR are described herein, the order can be as provided, but other orders are included as well. In other words, in an embodiment, the order is as set out in the text, but in other embodiments, the order can be different. E.g., the order of elements on one side of a transmembrane region can be different from the example, e.g., the placement of a switch domain relative to a intracellular signaling domain can be different, e.g., reversed).

In an embodiment, the first and second switch domains can form an intracellular or an extracellular dimerization switch. In an embodiment, the dimerization switch can be a homodimerization switch, e.g., where the first and second switch domain are the same, or a heterodimerization switch, e.g., where the first and second switch domain are different from one another.

In embodiments, an RCAR can comprise a “multi switch.” A multi switch can comprise heterodimerization switch domains or homodimerization switch domains. A multi switch comprises a plurality of, e.g., 2, 3, 4, 5, 6, 7, 8, 9, or 10, switch domains, independently, on a first member, e.g., an antigen binding member, and a second member, e.g., an intracellular signaling member. In an embodiment, the first member can comprise a plurality of first switch domains, e.g., FKBP-based switch domains, and the second member can comprise a plurality of second switch domains, e.g., FRB-based switch domains. In an embodiment, the first member can comprise a first and a second switch domain, e.g., a FKBP-based switch domain and a FRB-based switch domain, and the second member can comprise a first and a second switch domain, e.g., a FKBP-based switch domain and a FRB-based switch domain.

In an embodiment, the intracellular signaling member comprises one or more intracellular signaling domains, e.g., a primary intracellular signaling domain and one or more costimulatory signaling domains.

In an embodiment, the antigen binding member may comprise one or more intracellular signaling domains, e.g., one or more costimulatory signaling domains. In an embodiment, the antigen binding member comprises a plurality, e.g., 2 or 3 costimulatory signaling domains described herein, e.g., selected from 4-1BB, CD28, CD27, ICOS, and OX40, and in embodiments, no primary intracellular signaling domain. In an embodiment, the antigen binding member comprises the following costimulatory signaling domains, from the extracellular to intracellular direction: 4-1BB-CD27; 4-1BB-CD27; CD27-4-1BB; 4-1BB-CD28; CD28-4-1BB; OX40-CD28; CD28-OX40; CD28-4-1BB; or 4-1BB-CD28. In such embodiments, the intracellular binding member comprises a CD3zeta domain. In one such embodiment the RCAR comprises (1) an antigen binding member comprising, an antigen binding domain, a transmembrane domain, and two costimulatory domains and a first switch domain; and (2) an intracellular signaling domain comprising a transmembrane domain or membrane tethering domain and at least one primary intracellular signaling domain, and a second switch domain.

An embodiment provides RCARs wherein the antigen binding member is not tethered to the surface of the CAR cell. This allows a cell having an intracellular signaling member to be conveniently paired with one or more antigen binding domains, without transforming the cell with a sequence that encodes the antigen binding member. In such embodiments, the RCAR comprises: 1) an intracellular signaling member comprising: a first switch domain, a transmembrane domain, an intracellular signaling domain, e.g., a primary intracellular signaling domain, and a first switch domain; and 2) an antigen binding member comprising: an antigen binding domain, and a second switch domain, wherein the antigen binding member does not comprise a transmembrane domain or membrane tethering domain, and, optionally, does not comprise an intracellular signaling domain. In some embodiments, the RCAR may further comprise 3) a second antigen binding member comprising: a second antigen binding domain, e.g., a second antigen binding domain that binds a different antigen than is bound by the antigen binding domain; and a second switch domain.

Also provided herein are RCARs wherein the antigen binding member comprises bispecific activation and targeting capacity. In this embodiment, the antigen binding member can comprise a plurality, e.g., 2, 3, 4, or 5 antigen binding domains, e.g., scFvs, wherein each antigen binding domain binds to a target antigen, e.g. different antigens or the same antigen, e.g., the same or different epitopes on the same antigen. In an embodiment, the plurality of antigen binding domains are in tandem, and optionally, a linker or hinge region is disposed between each of the antigen binding domains. Suitable linkers and hinge regions are described herein.

An embodiment provides RCARs having a configuration that allows switching of proliferation. In this embodiment, the RCAR comprises: 1) an intracellular signaling member comprising: optionally, a transmembrane domain or membrane tethering domain; one or more co-stimulatory signaling domain, e.g., selected from 4-1BB, CD28, CD27, ICOS, and OX40, and a switch domain; and 2) an antigen binding member comprising: an antigen binding domain, a transmembrane domain, and a primary intracellular signaling domain, e.g., a CD3zeta domain, wherein the antigen binding member does not comprise a switch domain, or does not comprise a switch domain that dimerizes with a switch domain on the intracellular signaling member. In an embodiment, the antigen binding member does not comprise a co-stimulatory signaling domain. In an embodiment, the intracellular signaling member comprises a switch domain from a homodimerization switch. In an embodiment, the intracellular signaling member comprises a first switch domain of a heterodimerization switch and the RCAR comprises a second intracellular signaling member which comprises a second switch domain of the heterodimerization switch. In such embodiments, the second intracellular signaling member comprises the same intracellular signaling domains as the intracellular signaling member. In an embodiment, the dimerization switch is intracellular. In an embodiment, the dimerization switch is extracellular.

In any of the RCAR configurations described here, the first and second switch domains comprise a FKBP-FRB based switch as described herein.

Also provided herein are cells comprising an RCAR described herein. Any cell that is engineered to express a RCAR can be used as a RCARX cell. In an embodiment the RCARX cell is a T cell, and is referred to as a RCART cell. In an embodiment the RCARX cell is an NK cell, and is referred to as a RCARN cell.

Also provided herein are nucleic acids and vectors comprising RCAR encoding sequences. Sequence encoding various elements of an RCAR can be disposed on the same nucleic acid molecule, e.g., the same plasmid or vector, e.g., viral vector, e.g., lentiviral vector. In an embodiment, (i) sequence encoding an antigen binding member and (ii) sequence encoding an intracellular signaling member, can be present on the same nucleic acid, e.g., vector. Production of the corresponding proteins can be achieved, e.g., by the use of separate promoters, or by the use of a bicistronic transcription product (which can result in the production of two proteins by cleavage of a single translation product or by the translation of two separate protein products). In an embodiment, a sequence encoding a cleavable peptide, e.g., a P2A or F2A sequence, is disposed between (i) and (ii). In an embodiment, a sequence encoding an IRES, e.g., an EMCV or EV71 IRES, is disposed between (i) and (ii). In these embodiments, (i) and (ii) are transcribed as a single RNA. In an embodiment, a first promoter is operably linked to (i) and a second promoter is operably linked to (ii), such that (i) and (ii) are transcribed as separate mRNAs.

Alternatively, the sequence encoding various elements of an RCAR can be disposed on the different nucleic acid molecules, e.g., different plasmids or vectors, e.g., viral vector, e.g., lentiviral vector. E.g., the (i) sequence encoding an antigen binding member can be present on a first nucleic acid, e.g., a first vector, and the (ii) sequence encoding an intracellular signaling member can be present on the second nucleic acid, e.g., the second vector.

### Dimerization Switches

Dimerization switches can be non-covalent or covalent. In a non-covalent dimerization switch, the dimerization molecule promotes a non-covalent interaction between the switch domains. In a covalent dimerization switch, the dimerization molecule promotes a covalent interaction between the switch domains.

In an embodiment, the RCAR comprises a FKBP/FRAP, or FKBP/FRB,-based dimerization switch. FKBP12 (FKBP, or FK506 binding protein) is an abundant cytoplasmic protein that serves as the initial intracellular target for the natural product immunosuppressive drug, rapamycin. Rapamycin binds to FKBP and to the large PI3K homolog FRAP (RAFT, mTOR). FRB is a 93 amino acid portion of FRAP, that is sufficient for binding the FKBP-rapamycin complex (Chen, J., Zheng, X. F., Brown, E. J. & Schreiber, S. L. (1995) Identification of an 11-kDa FKBP12-rapamycin-binding domain within the 289-kDa FKBP12-rapamycin-associated protein and characterization of a critical serine residue. Proc Natl Acad Sci USA 92: 4947-51.)

In embodiments, an FKBP/FRAP, e.g., an FKBP/FRB, based switch can use a dimerization molecule, e.g., rapamycin or a rapamycin analog.

The amino acid sequence of FKBP is as follows:

(SEQ ID NO: 588)

D V P D Y A S L G G P S S P K K K R K V S R G V Q
V E T I S P G D G R T F P K R G Q T C V V H Y T G
M L E D G K K F D S S R D R N K P F K F M L G K Q
E V I R G W E E G V A Q M S V G Q R A K L T I S P
D Y A Y G A T G H P G I I P P H A T L V F D V E L
L K L E T S Y

In embodiments, an FKBP switch domain can comprise a fragment of FKBP having the ability to bind with FRB, or a fragment or analog thereof, in the presence of rapamycin or a rapalog, e.g., the underlined portion of SEQ ID NO: 588, which is:

(SEQ ID NO: 589)

V Q V E T I S P G D G R T F P K R G Q T C V V H Y
T G M L E D G K K F D S S R D R N K P F K F M L
G K Q E V I R G W E E G V A Q M S V G Q R A K L
T I S P D Y A Y G A T G H P G I I P P H A T L V F
D V E L L K L E T S

The amino acid sequence of FRB is as follows:

(SEQ ID NO: 590)

ILWHEMWHEG LEEASRLYFG ERNVKGMFEV LEPLHAMMER
GPQTLKETSF NQAYGRDLME AQEWCRKYMK SGNVKDLTQA
WDLYYHVFRR ISK

“FKBP/FRAP, e.g., an FKBP/FRB, based switch” as that term is used herein, refers to a dimerization switch comprising: a first switch domain, which comprises an FKBP fragment or analog thereof having the ability to bind with FRB, or a fragment or analog thereof, in the presence of rapamycin or a rapalog, e.g., RAD001, and has at least 70, 75, 80, 85, 90, 95, 96, 97, 98, or 99% identity with, or differs by no more than 30, 25, 20, 15, 10, 5, 4, 3, 2, or 1 amino acid residues from, the FKBP sequence of SEQ ID NO: 588 or 589; and a second switch domain, which comprises an FRB fragment or analog thereof having the ability to bind with FRB, or a fragment or analog thereof, in the presence of rapamycin or a rapalog, and has at least 70, 75, 80, 85, 90, 95, 96, 97, 98, or 99% identity with, or differs by no more than 30, 25, 20, 15, 10, 5, 4, 3, 2, or 1 amino acid residues from, the FRB sequence of SEQ ID NO: 590. In an embodiment, a RCAR described herein comprises one switch domain comprising amino acid residues disclosed in SEQ ID NO: 588 (or SEQ ID NO: 589), and one switch domain comprising amino acid residues disclosed in SEQ ID NO: 590.

In embodiments, the FKBP/FRB dimerization switch comprises a modified FRB switch domain that exhibits altered, e.g., enhanced, complex formation between an FRB-based switch domain, e.g., the modified FRB switch domain, a FKBP-based switch domain, and the dimerization molecule, e.g., rapamycin or a rapalogue, e.g., RAD001. In an embodiment, the modified FRB switch domain comprises one or more mutations, e.g., 2, 3, 4, 5, 6, 7, 8, 9, 10 or more, selected from mutations at amino acid position(s) L2031, E2032, S2035, R2036, F2039, G2040, T2098, W2101, D2102, Y2105, and F2108, where the wild-type amino acid is mutated to any other naturally-occurring amino acid. In an embodiment, a mutant FRB comprises a mutation at E2032, where E2032 is mutated to phenylalanine (E2032F), methionine (E2032M), arginine (E2032R), valine (E2032V), tyrosine (E2032Y), isoleucine (E2032I), e.g., SEQ ID NO: 591, or leucine (E2032L), e.g., SEQ ID NO: 592. In an embodiment, a mutant FRB comprises a mutation at T2098, where T2098 is mutated to phenylalanine (T2098F) or leucine (T2098L), e.g., SEQ ID NO: 593. In an embodiment, a mutant FRB comprises a mutation at E2032 and at T2098, where E2032 is mutated to any amino acid, and where T2098 is mutated to any amino acid, e.g., SEQ ID NO: 594. In an embodiment, a mutant FRB comprises an E2032I and a T2098L mutation, e.g., SEQ ID NO: 595. In an embodiment, a mutant FRB comprises an E2032L and a T2098L mutation, e.g., SEQ ID NO: 596.

TABLE 17A
Exemplary mutant FRB having increased affinity for a
dimerization molecule.

| | | SEQ |
|---|---|---|
| | | ID |
| FRB mutant | Amino Acid Sequence | NO: |
| E2032I mutant | ILWHEMWHEGLIEASRLYFGERNVKGMFEVLEPLHAMMER | 591 |
| | GPQTLKETSFNQAYGRDLMEAQEWCRKYMKSGNVKDLTQ | |
| | AWDLYYHVFRRISKTS | |
| E2032L mutant | ILWHEMWHEGLLEASRLYFGERNVKGMFEVLEPLHAMME | 592 |
| | RGPQTLKETSFNQAYGRDLMEAQEWCRKYMKSGNVKDLT | |
| | QAWDLYYHVFRRISKTS | |
| T2098 L mutant | ILWHEMWHEGLEEASRLYFGERNVKGMFEVLEPLHAMME | 593 |
| | RGPQTLKETSFNQAYGRDLMEAQEWCRKYMKSGNVKDLL | |
| | QAWDLYYHVFRRISKTS | |
| E2032, T2098 | ILWHEMWHEGLXEASRLYFGERNVKGMFEVLEPLHAMME | 594 |
| mutant | RGPQTLKETSFNQAYGRDLMEAQEWCRKYMKSGNVKDLX | |
| | QAWDLYYHVFRRISKTS | |
| E2032I, T2098L | ILWHEMWHEGLIEASRLYFGERNVKGMFEVLEPLHAMMER | 595 |
| mutant | GPQTLKETSFNQAYGRDLMEAQEWCRKYMKSGNVKDLLQ | |
| | AWDLYYHVFRRISKTS | |
| E2032L, | ILWHEMWHEGLLEASRLYFGERNVKGMFEVLEPLHAMME | 596 |
| T2098L | RGPQTLKETSFNQAYGRDLMEAQEWCRKYMKSGNVKDLL | |
| mutant | QAWDLYYHVFRRISKTS | |

Other suitable dimerization switches include a GyrB-GyrB based dimerization switch, a Gibberellin-based dimerization switch, a tag/binder dimerization switch, and a halo-tag/snap-tag dimerization switch. Following the guidance provided herein, such switches and relevant dimerization molecules will be apparent to one of ordinary skill.

### Dimerization Molecule

Association between the switch domains is promoted by the dimerization molecule. In the presence of dimerization molecule interaction or association between switch domains allows for signal transduction between a polypeptide associated with, e.g., fused to, a first switch domain, and a polypeptide associated with, e.g., fused to, a second switch domain. In the presence of non-limiting levels of dimerization molecule signal transduction is increased by 1.1, 1.2, 1.3, 1.4, 1.5, 1.6, 1.7, 1.8, 1.9, 2, 5, 10, 50, 100 fold, e.g., as measured in a system described herein.

Rapamycin and rapamycin analogs (sometimes referred to as rapalogues), e.g., RAD001, can be used as dimerization molecules in a FKBP/FRB-based dimerization switch described herein. In an embodiment the dimerization molecule can be selected from rapamycin (sirolimus), RAD001 (everolimus), zotarolimus, temsirolimus, AP-23573 (ridaforolimus), biolimus and AP21967. Additional rapamycin analogs suitable for use with FKBP/FRB-based dimerization switches are further described in the section entitled “Combination Therapies”, or in the subsection entitled “Combination with a low dose mTOR inhibitor”.

Co-Expression of CAR with a Chemokine Receptor

In embodiments, the CAR-expressing cell described herein further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, e.g., solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In one embodiment, the chemokine receptor molecule to be expressed with a CAR described herein is selected based on the chemokine(s) secreted by the tumor. In one embodiment, the CAR-expressing cell described herein further comprises, e.g., expresses, a CCR2b receptor or a CXCR2 receptor. In an embodiment, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### RNA Transfection

Disclosed herein are methods for producing an in vitro transcribed RNA CAR. The present invention also includes a CAR encoding RNA construct that can be directly transfected into a cell. A method for generating mRNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail, typically 50-2000 bases in length (SEQ ID NO:35). RNA so produced can efficiently transfect different kinds of cells. In one aspect, the template includes sequences for the CAR.

In one aspect the CAR described herein, e.g., CD123 CAR or CD19 CAR, is encoded by a messenger RNA (mRNA). In one aspect the mRNA encoding the CAR, e.g., CD123 CAR or CD19 CAR, is introduced into a T cell for production of a CART cell.

Additional methods of RNA transfection are described on pages 192-196 of International Application WO 2016/164731, filed Apr. 8, 2016, which is incorporated by reference in its entirety.

### Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome.

Additional and exemplary transposons and non-viral delivery methods are described on pages 196-198 of International Application WO 2016/164731, filed Apr. 8, 2016, which is incorporated by reference in its entirety.

### Nucleic Acid Constructs Encoding a CAR

In accordance with any method or composition described herein, a CAR can be encoded by a nucleic acid construct. Exemplary nucleic acid molecules encoding one or more CAR constructs are described herein. In embodiments, the nucleic acid molecule is provided as a messenger RNA transcript. In embodiments, the nucleic acid molecule is provided as a DNA construct.

In embodiments, the nucleic acid molecule comprises an isolated nucleic acid molecule encoding a chimeric antigen receptor (CAR), wherein the CAR comprises an antigen binding domain (e.g., CD123 or CD19 binding domain (e.g., a humanized or human CD123 or CD19 binding domain), a transmembrane domain, and an intracellular signaling domain comprising a stimulatory domain, e.g., a costimulatory signaling domain and/or a primary signaling domain, e.g., zeta chain.

In one embodiment, the antigen binding domain (e.g., CD123 binding domain) is an antigen binding domain (e.g., CD123 binding domain) described herein, e.g., an CD123 binding domain which comprises a sequence selected from a group consisting of SEQ ID NO: 157-160, 184-215, 478, 480, 483, 485, and 556-587, or a sequence with at least 95%, e.g., 95-99% identity thereof. In one embodiment, the CD123 binding domain comprises a human CD123 binding domain which comprises a sequence selected from a group consisting of SEQ ID NO: 157-160, 478, 480, 483, and 485. In one embodiment, the CD123 binding domain comprises a humanized CD123 binding domain which comprises a sequence selected from a group consisting of SEQ ID NO: 184-215, and 556-587.

In one embodiment, the anti-CD19 binding domain is an anti-CD19 binding domain described herein, e.g., an anti-CD19 binding domain which comprises a sequence selected from a group consisting of SEQ ID NO: 710-721, 734-745, 771, 774, 775, 777, or 780, or a sequence with at least 95%, e.g., 95-99% identify thereof.

In one embodiment, the transmembrane domain is transmembrane domain of a protein, e.g., described herein, e.g., selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154. In one embodiment, the transmembrane domain comprises a sequence of SEQ ID NO: 6, or a sequence with at least 95%, e.g., 95-99% identity thereof. In one embodiment, the CD123 binding domain is connected to the transmembrane domain by a hinge region, e.g., a hinge described herein. In one embodiment, the hinge region comprises SEQ ID NO:2 or SEQ ID NO:3 or SEQ ID NO:4 or SEQ ID NO:5, or a sequence with at least 95%, e.g., 95-99% identity thereof.

In one embodiment, the isolated nucleic acid molecule further comprises a sequence encoding a costimulatory domain. In one embodiment, the costimulatory domain is a functional signaling domain of a protein, e.g., described herein, e.g., selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In one embodiment, the costimulatory domain comprises a sequence of SEQ ID NO:7, or a sequence with at least 95%, e.g., 95-99%, identity thereof. In one embodiment, the intracellular signaling domain comprises a functional signaling domain of 4-1BB and a functional signaling domain of CD3 zeta. In one embodiment, the intracellular signaling domain comprises the sequence of SEQ ID NO: 7 or SEQ ID NO:8, or a sequence with at least 95%, e.g., 95-99%, identity thereof, and the sequence of SEQ ID NO: 9 or SEQ ID NO:10, or a sequence with at least 95%, e.g., 95-99%, identity thereof, wherein the sequences comprising the intracellular signaling domain are expressed in the same frame and as a single polypeptide chain.

In another aspect, the invention pertains to an isolated nucleic acid molecule encoding a CAR construct comprising a leader sequence of SEQ ID NO: 1, a scFv domain having a sequence selected from the group consisting of SEQ ID NOS: 157-160, 184-215, 478, 480, 483, 485, and 556-587 (or a sequence with at least 95%, e.g., 95-99%, identity thereof), a hinge region of SEQ ID NO:2 or SEQ ID NO:3 or SEQ ID NO:4 or SEQ ID NO:5 (or a sequence with at least 95%, e.g., 95-99%, identity thereof), a transmembrane domain having a sequence of SEQ ID NO: 6 (or a sequence with at least 95%, e.g., 95-99%, identity thereof), a 4-1BB costimulatory domain having a sequence of SEQ ID NO:7 or a CD27 costimulatory domain having a sequence of SEQ ID NO:8 (or a sequence with at least 95%, e.g., 95-99%, identity thereof)) or a CD28 costimulatory domain having a sequence of SEQ ID NO:43 (or a sequence with at least 95%, e.g., 95-99%, identity thereof) or a ICOS costimulatory domain having a sequence of SEQ ID NO: 45 (or a sequence with at least 95%, e.g., 95-99%, identity thereof), and a CD3 zeta stimulatory domain having a sequence of SEQ ID NO:9 or SEQ ID NO:10 (or a sequence with at least 95%, e.g., 95-99%, identity thereof).

In another aspect, the invention pertains to an isolated nucleic acid molecule encoding a CAR construct comprising a leader sequence of SEQ ID NO: 1, a scFv domain having a sequence selected from the group consisting of SEQ ID NO: 710-721, 734-745, 771, 774, 775, 777, and 780 (or a sequence with at least 95%, e.g., 95-99%, identify thereof), a hinge region of SEQ ID NO: 2, SEQ ID NO:3, SEQ ID NO:4, SEQ ID NO:5, SEQ ID NO: 16, or SEQ ID NO: 39 (or a sequence with at least 95%, e.g., 95-99%, identity thereof), a transmembrane domain having a sequence of SEQ ID NO: 6 (or a sequence with at least 95%, e.g., 95-99%, identity thereof), a 4-1BB costimulatory domain having a sequence of SEQ ID NO: 7 (or a sequence with at least 95%, e.g., 95-99%, identity thereof) or a CD27 costimulatory domain having a sequence of SEQ ID NO: 8 (or a sequence with at least 95%, e.g., 95-99%, identity thereof), and a CD3 zeta stimulatory domain having a sequence of SEQ ID NO: 9 or SEQ ID NO: 10 (or a sequence with at least 95%, e.g., 95-99%, identity thereof).

In another aspect, the invention pertains to an isolated polypeptide molecule encoded by the nucleic acid molecule. In one embodiment, the isolated polypeptide molecule comprises a sequence selected from the group consisting of SEQ ID NO: 98-101 and 125-156, or a sequence with at least 95%, e.g., 95-99%, identity thereof.

In another aspect, the invention pertains to an isolated polypeptide molecule encoded by the nucleic acid molecule. In one embodiment, the isolated polypeptide molecule comprises a sequence selected from the group consisting of SEQ ID NO: 758-769, 773, 776, 778, 779, and 781, or a sequence with at least 95%, e.g., 95-99%, identity thereof.

In another aspect, the invention pertains to a nucleic acid molecule encoding a chimeric antigen receptor (CAR) molecule that comprises a CD123 binding domain, a transmembrane domain, and an intracellular signaling domain comprising a stimulatory domain, and wherein said CD123 binding domain comprises a sequence selected from the group consisting of SEQ ID NO: 157-160, 184-215, 478, 480, 483, 485, and 556-587, or a sequence with at least 95%, e.g., 95-99%, identity thereof. In one embodiment, the CD123 binding domain comprises a human CD123 binding domain comprising a sequence selected from the group consisting of SEQ ID NO: 157-160, 478, 480, 483, and 485, or a sequence with at least 95%, e.g., 95-99%, identity thereof. In one embodiment, the CD123 binding domain comprises a humanized CD123 binding domain comprising a sequence selected from the group consisting of SEQ ID NO: 184-215, and 556-587, or a sequence with at least 95%, e.g., 95-99%, identity thereof. In another aspect, the invention pertains to an isolated nucleic acid molecule encoding a chimeric antigen receptor (CAR) molecule that comprises an anti-CD19 binding domain, a transmembrane domain, and an intracellular signaling domain comprising a stimulatory domain, and wherein the nucleic acid encoding the anti-CD19 binding domain comprises a sequence selected from the group consisting of SEQ ID NOs: 710-721, 734-745, 771, 774, 775, 777, and 780, or a sequence with at least 95%, e.g., 95-99%, identify thereof.

In one embodiment, the encoded CAR molecule further comprises a sequence encoding a costimulatory domain. In one embodiment, the costimulatory domain is a functional signaling domain of a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. In one embodiment, the costimulatory domain comprises a sequence of SEQ ID NO:7.

In one embodiment, the transmembrane domain is a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In one embodiment, the transmembrane domain comprises a sequence of SEQ ID NO:6. In one embodiment, the intracellular signaling domain comprises a functional signaling domain of 4-1BB and a functional signaling domain of zeta. In one embodiment, the intracellular signaling domain comprises the sequence of SEQ ID NO: 7 and the sequence of SEQ ID NO:9, wherein the sequences comprising the intracellular signaling domain are expressed in the same frame and as a single polypeptide chain. In one embodiment, the CD123 binding domain is connected to the transmembrane domain by a hinge region. In one embodiment, the hinge region comprises SEQ ID NO:2. In one embodiment, the hinge region comprises SEQ ID NO:3 or SEQ ID NO:4 or SEQ ID NO:5.

In another aspect, the invention pertains to an encoded CAR molecule comprising a leader sequence of SEQ ID NO: 1, a scFv domain having a sequence selected from the group consisting of SEQ ID NO: 157-160, 184-215, 478, 480, 483, 485, 556-587, or a sequence with at least 95%, e.g., 95-99%, identity thereof, a hinge region of SEQ ID NO:2 or SEQ ID NO:3 or SEQ ID NO:4 or SEQ ID NO:5, a transmembrane domain having a sequence of SEQ ID NO: 6, a 4-1BB costimulatory domain having a sequence of SEQ ID NO:7 or a CD27 costimulatory domain having a sequence of SEQ ID NO:8 or a CD28 costimulatory domain having a sequence of SEQ ID NO:43 or an ICOS costimulatory domain having a sequence of SEQ ID NO: 45, and a CD3 zeta stimulatory domain having a sequence of SEQ ID NO:9 or SEQ ID NO:10. In one embodiment, the encoded CAR molecule comprises a sequence selected from a group consisting of SEQ ID NO: 98-101 and 125-156, or a sequence with at least 95%, e.g., 95-99%, identity thereof.

In another aspect, the invention pertains to an isolated CAR molecule comprising a leader sequence of SEQ ID NO: 1, a scFv domain having a sequence selected from the group consisting of SEQ ID NOS: 710-721, 734-745, 771, 774, 775, 777, and 780, or a sequence with at least 95%, e.g., 95-99%, identify thereof, a hinge region of SEQ ID NO:2, SEQ ID NO: 3, SEQ ID NO: 4, SEQ ID NO: 5, SEQ ID NO: 16, or SEQ ID NO: 39, a transmembrane domain having a sequence of SEQ ID NO: 6, a 4-1BB costimulatory domain having a sequence of SEQ ID NO: 7 or a CD27 costimulatory domain having a sequence of SEQ ID NO: 8, and a CD3 zeta stimulatory domain having a sequence of SEQ ID NO: 9 or SEQ ID NO: 10. In one embodiment, the encoded CAR molecule comprises a sequence selected from the group consisting of SEQ ID NOS:710-721, 758-769, 771, and 773-792, or a sequence with at least 95%, e.g., 95-99%, identify thereof.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

### Vectors

The present invention also provides vectors in which a DNA of the present invention is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR of the invention is an adenoviral vector (A5/35). In another embodiment, the expression of nucleic acids encoding CARs can be accomplished using of transposons such as sleeping beauty, crisper, CAS9, and zinc finger nucleases. See below June et al. 2009Nature Reviews Immunology 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

An example of a promoter that is capable of expressing a CAR transgene in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from transgenes cloned into a lentiviral vector. See, e.g., Milone et al., Mol. Ther. 17(8): 1453-1464 (2009). In one aspect, the EF1a promoter comprises the sequence provided as SEQ ID NO:11.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1□ promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired. The nucleotide sequences of exemplary PGK promoters are provided below.

WT PGK Promoter

(SEQ ID NO: 597)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCGCGGCGACGCAAAGGGCCTTGGTGCGGGT
CTCGTCGGCGCAGGGACGCGTTTGGGTCCCGACGGAACCTTTTCCGCGTT
GGGGTTGGGGCACCATAAGCT

Exemplary Truncated PGK Promoters:

PGK100:

(SEQ ID NO: 598)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:

(SEQ ID NO: 599)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:

(SEQ ID NO: 600)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:

(SEQ ID NO: 601)

ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In one embodiment, the vector can further comprise a nucleic acid encoding a second CAR. In one embodiment, the second CAR includes an antigen binding domain to a target expressed on acute myeloid leukemia cells, such as, e.g., CD33, CD34, CLL-1, FLT3, or folate receptor beta. In one embodiment, the vector comprises a nucleic aicd sequence encoding a first CAR that targets a first antigen and includes an intracellular signaling domain having a costimulatory signaling domain but not a primary signaling domain, and a nucleic acid encoding a second CAR that targets a second, different, antigen and includes an intracellular signaling domain having a primary signaling domain but not a costimulatory signaling domain. In one embodiment, the vector comprises a nucleic acid encoding a first CD123 CAR that includes a CD123 binding domain, a transmembrane domain and a costimulatory domain and a nucleic acid encoding a second CAR that targets an antigen other than CD123 (e.g., an antigen expressed on AML cells, e.g., CD33, CD34, CLL-1, FLT3, or folate receptor beta) and includes an antigen binding domain, a transmembrane domain and a primary signaling domain. In another embodiment, the vector comprises a nucleic acid encoding a first CD123 CAR that includes a CD123 binding domain, a transmembrane domain and a primary signaling domain and a nucleic acid encoding a second CAR that targets an antigen other than CD123 (e.g., an antigen expressed on AML cells, e.g., CD33, CLL-1, CD34, FLT3, or folate receptor beta) and includes an antigen binding domain to the antigen, a transmembrane domain and a costimulatory signaling domain.

In one embodiment, the vector comprises a nucleic acid encoding a CD123 CAR described herein and a nucleic acid encoding an inhibitory CAR. In one embodiment, the inhibitory CAR comprises an antigen binding domain that binds an antigen found on normal cells but not cancer cells, e.g., normal cells that also express CD123. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta.

In embodiments, the vector may comprise two or more nucleic acid sequences encoding a CAR, e.g., a CD123 CAR described herein and a second CAR, e.g., an inhibitory CAR or a CAR that specifically binds to an antigen other than CD123 (e.g., an antigen expressed on AML cells, e.g., CLL-1, CD33, CD34, FLT3, or folate receptor beta). In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include the following, wherein the GSG residues are optional:

T2A:

(SEQ ID NO: 602)

(GSG) E G R G S L L T C G D V E E N P G P
P2A:

(SEQ ID NO: 603)

(GSG) A T N F S L L K Q A G D V E E N P G P
E2A:

(SEQ ID NO: 604)

(GSG) Q C T N Y A L L K L A G D V E S N P G P
F2A:

(SEQ ID NO: 605)

(GSG) V K Q T L N F D L L K L A G D V E S N P G P

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A preferred method for the introduction of a polynucleotide into a host cell is calcium phosphate transfection Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

The present invention further provides a vector comprising a CAR encoding nucleic acid molecule. In one aspect, a CAR vector can be directly transduced into a cell, e.g., an immune effector cell, e.g., a T cell or NK cell. In one aspect, the vector is a cloning or expression vector, e.g., a vector including, but not limited to, one or more plasmids (e.g., expression plasmids, cloning vectors, minicircles, minivectors, double minute chromosomes), retroviral and lentiviral vector constructs. In one aspect, the vector is capable of expressing the CAR construct in mammalian immune effector cells, e.g., mammalian T cells or mammalian NK cells. In one aspect, the mammalian T cell is a human T cell.

### Sources of Cells

Prior to expansion and genetic modification, a source of cells (e.g., immune effector cells, e.g., T cells or NK cells), is obtained from a subject. The term “subject” is intended to include living organisms in which an immune response can be elicited (e.g., mammals). Examples of subjects include humans, dogs, cats, mice, rats, and transgenic species thereof.

In embodiments, immune effector cells (e.g., a population of immune effector cells), e.g., T cells, are derived from (e.g., differentiated from) a stem cell, e.g., an embryonic stem cell or a pluripotent stem cell, e.g., an induced pluripotent stem cell (iPSC). In embodiments, the cells are autologous or allogeneic. In embodiments wherein the cells are allogeneic, the cells, e.g., derived from stem cells (e.g., iPSCs), are modified to reduce their alloreactivity. For example, the cells can be modified to reduce alloreactivity, e.g., by modifying (e.g., disrupting) their T cell receptor. In embodiments, a site specific nuclease can be used to disrupt the T cell receptor, e.g., after T-cell differentiation. In other examples, cells, e.g., T cells derived from iPSCs, can be generated from virus-specific T cells, which are less likely to cause graft-versus-host disease because of their recognition of a pathogen-derived antigen. In yet other examples, alloreactivity can be reduced, e.g., minimized, by generating iPSCs from common HLA haplotypes such that they are histocompatible with matched, unrelated recipient subjects. In yet other examples, alloreactivity can be reduced, e.g., minimized, by repressing HLA expression through genetic modification. For example, T cells derived from iPSCs can be processed as described in, e.g., Themeli et al. Nat. Biotechnol. 31.10(2013):928-35, incorporated herein by reference. In some examples, immune effector cells, e.g., T cells, derived from stem cells, can be processed/generated using methods described in WO2014/165707, incorporated herein by reference. Additional embodiments pertaining to allogeneic cells are described herein, e.g., in the “Allogeneic CAR Immune Effector Cells” section herein.

T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain aspects of the present invention, any number of immune effector cell (e.g., T cell or NK cell) lines available in the art, may be used. In certain aspects of the present invention, T cells can be obtained from a unit of blood collected from a subject using any number of techniques known to the skilled artisan, such as Ficoll™ separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and to place the cells in an appropriate buffer or media for subsequent processing steps. In one aspect of the invention, the cells are washed with phosphate buffered saline (PBS). In an alternative aspect, the wash solution lacks calcium and may lack magnesium or may lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one aspect, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+T cells, can be further isolated by positive or negative selection techniques. For example, in one aspect, T cells are isolated by incubation with anti-CD3/anti-CD28 (e.g., 3×28)-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be desirable to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depletion reagent from Miltenyi™. In one embodiment, the ratio of cells to CD25 depletion reagent is 1e7 cells to 20 uL, or 1e7 cells to15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product.

In an embodiment, a subject is pre-treated with cyclophosphamide prior to collection of cells for CAR-expressing cell product manufacturing, thereby reducing the risk of subject relapse to CAR-expressing cell treatment. In an embodiment, a subject is pre-treated with an anti-GITR antibody prior to collection of cells for CAR-expressing cell product manufacturing, thereby reducing the risk of subject relapse to CAR-expressing cell treatment.

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B7-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 2 billion cells/ml is used. In one aspect, a concentration of 1 billion cells/ml is used. In a further aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10e6/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as immune effector cells, e.g., T cells or NK cells, isolated and frozen for later use in T cell therapy for any number of diseases or conditions that would benefit from cell therapy, e.g., T cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the immune effector cells, e.g., T cells or NK cells, may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

### Allogeneic CAR Immune Effector Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR (e.g., engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some aspects, downregulation of HLA may be accomplished by reducing or eliminating expression of beta-2 microglobulin (B2M).

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not expresses or expresses at low levels an inhibitory molecule, e.g. by any mehod described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell.

Expression of siRNA and shRNAs in T cells can be achieved using any conventional expression system, e.g., such as a lentiviral expression system.

Exemplary shRNAs that downregulate expression of one or more components of the TCR are described, e.g., in US Publication No.: 2012/0321667. Exemplary siRNA and shRNA that downregulate expression of HLA class I and/or HLA class II genes are described, e.g., in U.S. publication No.: US 2007/0036773.

CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta).

Naturally-occurring CRISPR/Cas systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

The CRISPR/Cas system has been modified for use in gene editing (silencing, enhancing or changing specific genes) in eukaryotes such as mice or primates. Wiedenheft et al. (2012) Nature 482: 331-8. This is accomplished by introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence; in the TCR and/or HLA CRISPR/Cas system, the spacers are derived from the TCR or HLA gene sequence.

RNA from the CRISPR locus is constitutively expressed and processed by Cas proteins into small RNAs. These comprise a spacer flanked by a repeat sequence. The RNAs guide other Cas proteins to silence exogenous genetic elements at the RNA or DNA level. Horvath et al. (2010) Science 327: 167-170; Makarova et al. (2006) Biology Direct 1: 7. The spacers thus serve as templates for RNA molecules, analogously to siRNAs. Pennisi (2013) Science 341: 833-836.

As these naturally occur in many different types of bacteria, the exact arrangements of the CRISPR and structure, function and number of Cas genes and their product differ somewhat from species to species. Haft et al. (2005) PLoS Comput. Biol. 1: e60; Kunin et al. (2007) Genome Biol. 8: R61; Mojica et al. (2005) J. Mol. Evol. 60: 174-182; Bolotin et al. (2005) Microbial. 151: 2551-2561; Pourcel et al. (2005) Microbial. 151: 653-663; and Stern et al. (2010) Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. (2008) Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi (2013) Science 341: 833-836.

The CRISPR/Cas system can thus be used to edit a TCR and/or HLA gene (adding or deleting a basepair), or introducing a premature stop which thus decreases expression of a TCR and/or HLA. The CRISPR/Cas system can alternatively be used like RNA interference, turning off TCR and/or HLA gene in a reversible fashion. In a mammalian cell, for example, the RNA can guide the Cas protein to a TCR and/or HLA promoter, sterically blocking RNA polymerases.

Artificial CRISPR/Cas systems can be generated which inhibit TCR and/or HLA, using technology known in the art, e.g., that described in U.S. Publication No. 20140068797 and Cong (2013) Science 339: 819-823. Other artificial CRISPR/Cas systems that are known in the art may also be generated which inhibit TCR and/or HLA, e.g., that described in Tsai (2014) Nature Biotechnol., 32:6 569-576, U.S. Pat. Nos. 8,871,445; 8,865,406; 8,795,965; 8,771,945; and 8,697,359.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta).

TALENs are produced artificially by fusing a TAL effector DNA binding domain to a DNA cleavage domain. Transcription activator-like effects (TALEs) can be engineered to bind any desired DNA sequence, including a portion of the HLA or TCR gene. By combining an engineered TALE with a DNA cleavage domain, a restriction enzyme can be produced which is specific to any desired DNA sequence, including a HLA or TCR sequence. These can then be introduced into a cell, wherein they can be used for genome editing. Boch (2011) Nature Biotech. 29: 135-6; and Boch et al. (2009) Science 326: 1509-12; Moscou et al. (2009) Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is a wild-type or mutated FokI endonuclease. Several mutations to FokI have been made for its use in TALENs; these, for example, improve cleavage specificity or activity. Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. (2011) Nature Biotech. 29: 143-8.

A HLA or TCR TALEN can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN; depending on the sequences of the foreign DNA and chromosomal sequence, this process can be used to correct a defect in the HLA or TCR gene or introduce such a defect into a wt HLA or TCR gene, thus decreasing expression of HLA or TCR.

TALENs specific to sequences in HLA or TCR can be constructed using any method known in the art, including various schemes using modular components. Zhang et al. (2011) Nature Biotech. 29: 149-53; Geibler et al. (2011) PLoS ONE 6: e19509.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAGS, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF beta).

Like a TALEN, a ZFN comprises a FokI nuclease domain (or derivative thereof) fused to a DNA-binding domain. In the case of a ZFN, the DNA-binding domain comprises one or more zinc fingers. Carroll et al. (2011) Genetics Society of America 188: 773-782; and Kim et al. (1996) Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs are required to target non-palindromic DNA sites. The two individual ZFNs must bind opposite strands of the DNA with their nucleases properly spaced apart. Bitinaite et al. (1998) Proc. Natl. Acad. Sci. USA 95: 10570-5.

Also like a TALEN, a ZFN can create a double-stranded break in the DNA, which can create a frame-shift mutation if improperly repaired, leading to a decrease in the expression and amount of HLA and/or TCR in a cell. ZFNs can also be used with homologous recombination to mutate in the HLA or TCR gene.

ZFNs specific to sequences in HLA AND/OR TCR can be constructed using any method known in the art. Cathomen et al. (2008) Mol. Ther. 16: 1200-7; Guo et al. (2010) J. Mol. Biol. 400: 96; U.S. Patent Publication 2011/0158957; and U.S. Patent Publication 2012/0060230.

### Telomerase Expression

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in an embodiment, an immune effector cell, e.g., a T cell, ectopically expresses a telomerase subunit, e.g., the catalytic subunit of telomerase, e.g., TERT, e.g., hTERT. In some aspects, this disclosure provides a method of producing a CAR-expressing cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a CAR.

In one aspect, the disclosure features a method of making a population of immune effector cells (e.g., T cells, NK cells). In an embodiment, the method comprises: providing a population of immune effector cells (e.g., T cells or NK cells), contacting the population of immune effector cells with a nucleic acid encoding a CAR; and contacting the population of immune effector cells with a nucleic acid encoding a telomerase subunit, e.g., hTERT, under conditions that allow for CAR and telomerase expression.

In an embodiment, the nucleic acid encoding the telomerase subunit is DNA. In an embodiment, the nucleic acid encoding the telomerase subunit comprises a promoter capable of driving expression of the telomerase subunit.

In an embodiment, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795) as follows:

(SEQ ID NO: 606)

MPRAPRCRAVRSLLRSHYREVLPLATFVRRLGPQGWRLVQRGDPAAFRAL
VAQCLVCVPWDARPPPAAPSFRQVSCLKELVARVLQRLCERGAKNVLAFG
FALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCE
RAWNHSVREAGVPLGLPAPGARRRGGSASRSLPLPKRPRRGAAPEPERTP
VGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSL
RPSLTGARRLVETIFLGSRPWMPGTPRRLPRLPQRYWQMRPLFLELLGNH
AQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKH
AKLSLQELTWKMSVRGCAWLRRSPGVGCVPAAEHRLREEILAKFLHWLMS
VYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKR
AERLTSRVKALFSVLNYERARRPGLLGASVLGLDDIHRAWRTFVLRVRAQ
DPPPELYFVKVDVTGAYDTIPQDRLTEVIASIIKPQNTYCVRRYAVVQKA
AHGHVRKAFKSHVSTLTDLQPYMRQFVAHLQETSPLRDAVVIEQSSSLNE
ASSGLFDVFLRFMCHHAVRIRGKSYVQCQGIPQGSILSTLLCSLCYGDME
NKLFAGIRRDGLLLRLVDDFLLVTPHLTHAKTFLRTLVRGVPEYGCVVNL
RKTVVNFPVEDEALGGTAFVQMPAHGLFPWCGLLLDTRTLEVQSDYSSYA
RTSIRASLTFNRGFKAGRNMRRKLFGVLRLKCHSLFLDLQVNSLQTVCTN
IYKILLLQAYRFHACVLQLPFHQQVWKNPTFFLRVISDTASLCYSILKAK
NAGMSLGAKGAAGPLPSEAVQWLCHQAFLLKLTRHRVTYVPLLGSLRTAQ
TQLSRKLPGTTLTALEAAANPALPSDFKTILD

In an embodiment, the hTERT has a sequence at least 80%, 85%, 90%, 95%, 96%, 97%, 98%, or 99% identical to the sequence of SEQ ID NO: 606. In an embodiment, the hTERT has a sequence of SEQ ID NO: 606. In an embodiment, the hTERT comprises a deletion (e.g., of no more than 5, 10, 15, 20, or 30 amino acids) at the N-terminus, the C-terminus, or both. In an embodiment, the hTERT comprises a transgenic amino acid sequence (e.g., of no more than 5, 10, 15, 20, or 30 amino acids) at the N-terminus, the C-terminus, or both.

In an embodiment, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795):

| (SEQ ID NO: 607) | |
|---|---|
| 1 | caggcagcgt ggtcctgctg cgcacgtggg aagccctggc cccggccacc cccgcgatgc |
| 61 | cgcgcgctcc ccgctgccga gccgtgcgct ccctgctgcg cagccactac cgcgaggtgc |
| 121 | tgccgctggc cacgttcgtg cggcgcctgg ggccccaggg ctggcggctg gtgcagcgcg |
| 181 | gggacccggc ggctttccgc gcgctggtgg cccagtgcct ggtgtgcgtg ccctgggacg |
| 241 | cacggccgcc ccccgccgcc ccctccttcc gccaggtgtc ctgcctgaag gagctggtgg |
| 301 | cccgagtgct gcagaggctg tgcgagcgcg gcgcgaagaa cgtgctggcc ttcggcttcg |
| 361 | cgctgctgga cggggcccgc gggggccccc ccgaggcctt caccaccagc gtgcgcagct |
| 421 | acctgcccaa cacggtgacc gacgcactgc gggggagcgg ggcgtggggg ctgctgttgc |
| 481 | gccgcgtggg cgacgacgtg ctggttcacc tgctggcacg ctgcgcgctc tttgtgctgg |
| 541 | tggctcccag ctgcgcctac caggtgtgcg ggccgccgct gtaccagctc ggcgctgcca |
| 601 | ctcaggcccg gcccccgcca cacgctagtg gaccccgaag gcgtctggga tgcgaacggg |
| 661 | cctggaacca tagcgtcagg gaggccgggg tccccctggg cctgccagcc ccgggtgcga |
| 721 | ggaggcgcgg gggcagtgcc agccgaagtc tgccgttgcc caagaggccc aggcgtggcg |
| 781 | ctgcccctga gccggagcgg acgcccgttg ggcaggggtc ctgggcccac ccgggcagga |
| 841 | cgcgtggacc gagtgaccgt ggtttctgtg tggtgtcacc tgccagaccc gccgaagaag |
| 901 | ccacctcttt ggagggtgcg ctctctggca cgcgccactc ccacccatcc gtgggccgcc |
| 961 | agcaccacgc gggcccccca tccacatcgc ggccaccacg tccctgggac acgccttgtc |
| 1021 | ccccggtgta cgccgagacc aagcacttcc tctactcctc aggcgacaag gagcagctgc |
| 1081 | ggccctcctt cctactcagc tctctgaggc ccagcctgac tggcgctcgg aggctcgtgg |
| 1141 | agaccatctt tctgggttcc aggccctgga tgccagggac tccccgcagg ttgccccgcc |
| 1201 | tgccccagcg ctactggcaa atgcggcccc tgtttctgga gctgcttggg aaccacgcgc |
| 1261 | agtgccccta cggggtgctc ctcaagacgc actgcccgct gcgagctgcg gtcaccccag |
| 1321 | cagccggtgt ctgtgcccgg gagaagcccc agggctctgt ggcggccccc gaggaggagg |
| 1381 | acacagaccc ccgtcgcctg gtgcagctgc tccgccagca cagcagcccc tggcaggtgt |
| 1441 | acggcttcgt gcgggcctgc ctgcgccggc tggtgccccc aggcctctgg ggctccaggc |
| 1501 | acaacgaacg ccgcttcctc aggaacacca agaagttcat ctccctgggg aagcatgcca |
| 1561 | agctctcgct gcaggagctg acgtggaaga tgagcgtgcg gggctgcgct tggctgcgca |
| 1621 | ggagcccagg ggttggctgt gttccggccg cagagcaccg tctgcgtgag gagatcctgg |
| 1681 | ccaagttcct gcactggctg atgagtgtgt acgtcgtcga gctgctcagg tctttctttt |
| 1741 | atgtcacgga gaccacgttt caaaagaaca ggctcttttt ctaccggaag agtgtctgga |
| 1801 | gcaagttgca aagcattgga atcagacagc acttgaagag ggtgcagctg cgggagctgt |
| 1861 | cggaagcaga ggtcaggcag catcgggaag ccaggcccgc cctgctgacg tccagactcc |
| 1921 | gcttcatccc caagcctgac gggctgcggc cgattgtgaa catggactac gtcgtgggag |
| 1981 | ccagaacgtt ccgcagagaa aagagggccg agcgtctcac ctcgagggtg aaggcactgt |
| 2041 | tcagcgtgct caactacgag cgggcgcggc gccccggcct cctgggcgcc tctgtgctgg |
| 2101 | gcctggacga tatccacagg gcctggcgca ccttcgtgct gcgtgtgcgg gcccaggacc |
| 2161 | cgccgcctga gctgtacttt gtcaaggtgg atgtgacggg cgcgtacgac accatccccc |
| 2221 | aggacaggct cacggaggtc atcgccagca tcatcaaacc ccagaacacg tactgcgtgc |
| 2281 | gtcggtatgc cgtggtccag aaggccgccc atgggcacgt ccgcaaggcc ttcaagagcc |
| 2341 | acgtctctac cttgacagac ctccagccgt acatgcgaca gttcgtggct cacctgcagg |
| 2401 | agaccagccc gctgagggat gccgtcgtca tcgagcagag ctcctccctg aatgaggcca |
| 2461 | gcagtggcct cttcgacgtc ttcctacgct tcatgtgcca ccacgccgtg cgcatcaggg |
| 2521 | gcaagtccta cgtccagtgc caggggatcc cgcagggctc catcctctcc acgctgctct |
| 2581 | gcagcctgtg ctacggcgac atggagaaca agctgtttgc ggggattcgg cgggacgggc |
| 2641 | tgctcctgcg tttggtggat gatttcttgt tggtgacacc tcacctcacc cacgcgaaaa |
| 2701 | ccttcctcag gaccctggtc cgaggtgtcc ctgagtatgg ctgcgtggtg aacttgcgga |
| 2761 | agacagtggt gaacttccct gtagaagacg aggccctggg tggcacggct tttgttcaga |
| 2821 | tgccggccca cggcctattc ccctggtgcg gcctgctgct ggatacccgg accctggagg |
| 2881 | tgcagagcga ctactccagc tatgcccgga cctccatcag agccagtctc accttcaacc |
| 2941 | gcggcttcaa ggctgggagg aacatgcgtc gcaaactctt tggggtcttg cggctgaagt |
| 3001 | gtcacagcct gtttctggat ttgcaggtga acagcctcca gacggtgtgc accaacatct |
| 3061 | acaagatcct cctgctgcag gcgtacaggt ttcacgcatg tgtgctgcag ctcccatttc |
| 3121 | atcagcaagt ttggaagaac cccacatttt tcctgcgcgt catctctgac acggcctccc |
| 3181 | tctgctactc catcctgaaa gccaagaacg cagggatgtc gctgggggcc aagggcgccg |
| 3241 | ccggccctct gccctccgag gccgtgcagt ggctgtgcca ccaagcattc ctgctcaagc |
| 3301 | tgactcgaca ccgtgtcacc tacgtgccac tcctggggtc actcaggaca gcccagacgc |
| 3361 | agctgagtcg gaagctcccg gggacgacgc tgactgccct ggaggccgca gccaacccgg |
| 3421 | cactgccctc agacttcaag accatcctgg actgatggcc acccgcccac agccaggccg |
| 3481 | agagcagaca ccagcagccc tgtcacgccg ggctctacgt cccagggagg gaggggcggc |
| 3541 | ccacacccag gcccgcaccg ctgggagtct gaggcctgag tgagtgtttg gccgaggcct |
| 3601 | gcatgtccgg ctgaaggctg agtgtccggc tgaggcctga gcgagtgtcc agccaagggc |
| 3661 | tgagtgtcca gcacacctgc cgtcttcact tccccacagg ctggcgctcg gctccacccc |
| 3721 | agggccagct tttcctcacc aggagcccgg cttccactcc ccacatagga atagtccatc |
| 3781 | cccagattcg ccattgttca cccctcgccc tgccctcctt tgccttccac ccccaccatc |
| 3841 | caggtggaga ccctgagaag gaccctggga gctctgggaa tttggagtga ccaaaggtgt |
| 3901 | gccctgtaca caggcgagga ccctgcacct ggatgggggt ccctgtgggt caaattgggg |
| 3961 | ggaggtgctg tgggagtaaa atactgaata tatgagtttt tcagttttga aaaaaaaaaa |
| 4021 | aaaaaaa |

In an embodiment, the hTERT is encoded by a nucleic acid having a sequence at least 80%, 85%, 90%, 95%, 96, 97%, 98%, or 99% identical to the sequence of SEQ ID NO: 607. In an embodiment, the hTERT is encoded by a nucleic acid of SEQ ID NO: 607.

### Activation and Expansion of Cells

Cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain aspects of the invention, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In one particular aspect, a preferred particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects of the present invention, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells, e.g., a CD123 CAR cell described herein or a CD19 CAR cell described herein, are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a CD123 CAR cell described herein or a CD19 CAR cell described herein, expanded for 5 days show at least a one, two, three or four fold increase in cells doublings upon antigen stimulation as compared to the same cells expanded in culture for 9 days under the same culture conditions. In one embodiment, the cells, e.g., the cells expressing a CD123 CAR described herein or a CD19 CAR cell described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CD123 CAR cell described herein or a CD19 CAR cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

In one aspect of the present invention, the mixture may be cultured for several hours (about 3 hours) to about 14 days or any hourly integer value in between. In one aspect, the mixture may be cultured for 21 days. In one aspect of the invention the beads and the T cells are cultured together for about eight days. In one aspect, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence of IL-15 and/or IL-7 (e.g., IL-15 and IL-7).

In embodiments, methods described herein, e.g., CAR-expressing cell manufacturing methods, comprise removing T regulatory cells, e.g., CD25+ T cells, from a cell population, e.g., using an anti-CD25 antibody, or fragment thereof, or a CD25-binding ligand, IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell described herein is contacted with a composition comprising hetIL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Ra polypeptide during ex vivo expansion. In one embodiment the contacting results in the survival and proliferation of a lymphocyte subpopulation, e.g., CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR (e.g., CAR described herein, e.g., CD123 CAR or CD19 CAR) is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR (e.g., CAR described herein, e.g., CD123 CAR or CD19 CAR) are described in further detail below.

Western blot analysis of CAR expression in primary T cells can be used to detect the presence of monomers and dimers. See, e.g., Milone et al., Molecular Therapy 17(8): 1453-1464 (2009). Very briefly, T cells (1:1 mixture of CD4⁺ and CD8⁺ T cells) expressing the CARs are expanded in vitro for more than 10 days followed by lysis and SDS-PAGE under reducing conditions. CARs containing the full length TCR-t cytoplasmic domain and the endogenous TCR-t chain are detected by western blotting using an antibody to the TCR-t chain. The same T cell subsets are used for SDS-PAGE analysis under non-reducing conditions to permit evaluation of covalent dimer formation.

In vitro expansion of CAR⁺ T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8⁺ T cell subsets by flow cytometry. See, e.g., Milone et al., Molecular Therapy 17(8): 1453-1464 (2009). Alternatively, a mixture of CD4⁺ and CD8⁺ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either CD19⁺ K562 cells (K562-CD19), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/ml. GFP T cells are enumerated by flow cytometry using bead-based counting. See, e.g., Milone et al., Molecular Therapy 17(8): 1453-1464 (2009). Similar assays can be performed using T cells that recognize other antigens (e.g., anti-CD123 T cells) (see, e.g. Gill et al Blood 2014; 123:2343) or with CART cells against other antigens (e.g., anti-CD123 CAR T cells).

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR on day 1.

Animal models can also be used to measure a CART activity. For example, xenograft model using human CD19-specific CAR⁺ T cells to treat a primary human pre-B ALL in immunodeficient mice can be used. See, e.g., Milone et al., Molecular Therapy 17(8): 1453-1464 (2009). Very briefly, after establishment of ALL, mice are randomized as to treatment groups. Different numbers of αCD19-ζ and αCD19-BB-ζ engineered T cells are coinjected at a 1:1 ratio into NOD-SCID-γ^{-/-} mice bearing B-ALL. The number of copies of αCD19-ζ and αCD19-BB-ζ vector in spleen DNA from mice is evaluated at various times following T cell injection. Animals are assessed for leukemia at weekly intervals. Peripheral blood CD19⁺ B-ALL blast cell counts are measured in mice that are injected with αCD19-ζ CAR⁺ T cells or mock-transduced T cells. Survival curves for the groups are compared using the log-rank test. In addition, absolute peripheral blood CD4⁺ and CD8⁺ T cell counts 4 weeks following T cell injection in NOD-SCID-γ^{-/-} mice can also be analyzed. Mice are injected with leukemic cells and 3 weeks later are injected with T cells engineered to express CAR by a bicistronic lentiviral vector that encodes the CAR linked to eGFP. T cells are normalized to 45-50% input GFP⁺ T cells by mixing with mock-transduced cells prior to injection, and confirmed by flow cytometry. Animals are assessed for leukemia at 1-week intervals. Survival curves for the CAR⁺ T cell groups are compared using the log-rank test. Similar experiments can be done with other CARTs, e.g., CD123 CARTs.

Dose dependent CAR treatment response can be evaluated. See, e.g., Milone et al., Molecular Therapy 17(8): 1453-1464 (2009). For example, peripheral blood is obtained 35-70 days after establishing leukemia in mice injected on day 21 with CAR T cells, an equivalent number of mock-transduced T cells, or no T cells. Mice from each group are randomly bled for determination of peripheral blood CD19⁺ ALL blast counts and then killed on days 35 and 49. The remaining animals are evaluated on days 57 and 70. Similar experiments can be done with other CARTs, e.g., CD123 CARTs.

Assessment of cell proliferation and cytokine production has been previously described, e.g., at Milone et al., Molecular Therapy 17(8): 1453-1464 (2009). Briefly, assessment of CAR-mediated proliferation is performed in microtiter plates by mixing washed T cells with K562 cells expressing CD19 (K19) or CD32 and CD137 (KT32-BBL) for a final T-cell:K562 ratio of 2:1. K562 cells are irradiated with gamma-radiation prior to use. Anti-CD3 (clone OKT3) and anti-CD28 (clone 9.3) monoclonal antibodies are added to cultures with KT32-BBL cells to serve as a positive control for stimulating T-cell proliferation since these signals support long-term CD8⁺ T cell expansion ex vivo. T cells are enumerated in cultures using CountBright™ fluorescent beads (Invitrogen, Carlsbad, Calif.) and flow cytometry as described by the manufacturer. CAR⁺ T cells are identified by GFP expression using T cells that are engineered with eGFP-2A linked CAR-expressing lentiviral vectors. For CAR+ T cells not expressing GFP, the CAR+ T cells are detected with biotinylated recombinant antigen (e.g., CD123 protein or CD19 protein) and a secondary avidin-PE conjugate. CD4+ and CD8⁺ expression on T cells are also simultaneously detected with specific monoclonal antibodies (BD Biosciences). Cytokine measurements are performed on supernatants collected 24 hours following re-stimulation using the human TH1/TH2 cytokine cytometric bead array kit (BD Biosciences, San Diego, Calif.) according the manufacturer's instructions or using a Luminex 30-plex kit (Invitrogen). Fluorescence is assessed using a BD Fortessa flow cytometer, and data is analyzed according to the manufacturer's instructions. Similar experiments can be done with other CARTs, e.g., CD123 CARTs.

Cytotoxicity can be assessed by a standard 51Cr-release assay. See, e.g., Milone et al., Molecular Therapy 17(8): 1453-1464 (2009). Briefly, target cells (K562 lines and primary pro-B-ALL cells) are loaded with 51Cr (as NaCrO₄, New England Nuclear, Boston, Mass.) at 37° C. for 2 hours with frequent agitation, washed twice in complete RPMI and plated into microtiter plates. Effector T cells are mixed with target cells in the wells in complete RPMI at varying ratios of effector cell:target cell (E:T). Additional wells containing media only (spontaneous release, SR) or a 1% solution of triton-X 100 detergent (total release, TR) are also prepared. After 4 hours of incubation at 37° C., supernatant from each well is harvested. Released 51Cr is then measured using a gamma particle counter (Packard Instrument Co., Waltham, Mass.). Each condition is performed in at least triplicate, and the percentage of lysis is calculated using the formula: % Lysis=(ER−SR)/(TR−SR), where ER represents the average 51Cr released for each experimental condition.

Imaging technologies can be used to evaluate specific trafficking and proliferation of CARs in tumor-bearing animal models. Such assays have been described, for example, in Barrett et al., Human Gene Therapy 22:1575-1586 (2011). Briefly, NOD/SCID/γc^{-/-} (NSG) mice are injected IV with Nalm-6 cells followed 7 days later with T cells 4 hour after electroporation with the CAR constructs. The T cells are stably transfected with a lentiviral construct to express firefly luciferase, and mice are imaged for bioluminescence. Alternatively, therapeutic efficacy and specificity of a single injection of CAR⁺ T cells in Nalm-6 xenograft model can be measured as the following: NSG mice are injected with Nalm-6 transduced to stably express firefly luciferase, followed by a single tail-vein injection of T cells electroporated with a CAR (e.g., CD123 CAR or CD19 CAR) 7 days later. Animals are imaged at various time points post injection. For example, photon-density heat maps of firefly luciferasepositive leukemia in representative mice at day 5 (2 days before treatment) and day 8 (24 hr post CAR⁺ PBLs) can be generated.

Other assays, including those described in the Example section of US2016/0068601A1 (incorporated herein by reference) as well as those that are known in the art can also be used to evaluate the CAR (e.g., CD123 CAR or CD19 CAR) constructs described herein.

Alternatively, or in combination to the methods disclosed herein, methods and compositions for one or more of: detection and/or quantification of CAR-expressing cells (e.g., in vitro or in vivo (e.g., clinical monitoring)); immune cell expansion and/or activation; and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that binds to a constant region of the extracellular binding domain). In other embodiments, the CAR ligand is a CAR antigen molecule (e.g., a CAR antigen molecule as described herein).

In one aspect, a method for detecting and/or quantifying CAR-expressing cells is disclosed. For example, the CAR ligand can be used to detect and/or quantify CAR-expressing cells in vitro or in vivo (e.g., clinical monitoring of CAR-expressing cells in a patient, or dosing a patient). The method includes:

providing the CAR ligand (optionally, a labelled CAR ligand, e.g., a CAR ligand that includes a tag, a bead, a radioactive or fluorescent label);

acquiring the CAR-expressing cell (e.g., acquiring a sample containing CAR-expressing cells, such as a manufacturing sample or a clinical sample);

contacting the CAR-expressing cell with the CAR ligand under conditions where binding occurs, thereby detecting the level (e.g., amount) of the CAR-expressing cells present. Binding of the CAR-expressing cell with the CAR ligand can be detected using standard techniques such as FACS, ELISA and the like.

In another aspect, a method of expanding and/or activating cells (e.g., immune effector cells) is disclosed. The method includes:

providing a CAR-expressing cell (e.g., a first CAR-expressing cell or a transiently expressing CAR cell);

contacting said CAR-expressing cell with a CAR ligand, e.g., a CAR ligand as described herein), under conditions where immune cell expansion and/or proliferation occurs, thereby producing the activated and/or expanded cell population.

In certain embodiments, the CAR ligand is present on (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In one embodiment, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In yet another aspect, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference. In one embodiment, the anti-idiotypic antibody molecule recognizes an anti-CD19 antibody molecule, e.g., an anti-CD19 scFv. For instance, the anti-idiotypic antibody molecule can compete for binding with the CD19-specific CAR mAb clone no. 136.20.1 described in Jena et al., PLOS March 2013 8:3 e57838; may have the same CDRs (e.g., one or more of, e.g., all of, VH CDR1, VH CDR2, CH CDR3, VL CDR1, VL CDR2, and VL CDR3, using the Kabat definition, the Chothia definition, or a combination of tthe Kabat and Chothia definitions) as the CD19-specific CAR mAb clone no. 136.20.1; may have one or more (e.g., 2) variable regions as the CD19-specific CAR mAb clone no. 136.20.1, or may comprise the CD19-specific CAR mAb clone no. 136.20.1. In some embodiments, the anti-idiotypic antibody was made according to a method described in Jena et al. In another embodiment, the anti-idiotypic antibody molecule is an anti-idiotypic antibody molecule described in WO 2014/190273. In some embodiments, the anti-idiotypic antibody molecule has the same CDRs (e.g., one or more of, e.g., all of, VH CDR1, VH CDR2, CH CDR3, VL CDR1, VL CDR2, and VL CDR3) as an antibody molecule of WO 2014/190273 such as 136.20.1; may have one or more (e.g., 2) variable regions of an antibody molecule of WO 2014/190273, or may comprise an antibody molecule of WO 2014/190273 such as 136.20.1. In other embodiments, the anti-CAR antibody binds to a constant region of the extracellular binding domain of the CAR molecule, e.g., as described in WO 2014/190273. In some embodiments, the anti-CAR antibody binds to a constant region of the extracellular binding domain of the CAR molecule, e.g., a heavy chain constant region (e.g., a CH2-CH3 hinge region) or light chain constant region. For instance, in some embodiments the anti-CAR antibody competes for binding with the 2D3 monoclonal antibody described in WO 2014/190273, has the same CDRs (e.g., one or more of, e.g., all of, VH CDR1, VH CDR2, CH CDR3, VL CDR1, VL CDR2, and VL CDR3) as 2D3, or has one or more (e.g., 2) variable regions of 2D3, or comprises 2D3 as described in WO 2014/190273.

In some aspects and embodiments, the compositions and methods herein are optimized for a specific subset of T cells, e.g., as described in U.S. Ser. No. 62/031,699 filed Jul. 31, 2014, the contents of which are incorporated herein by reference in their entirety. In some embodiments, the optimized subsets of T cells display an enhanced persistence compared to a control T cell, e.g., a T cell of a different type (e.g., CD8⁺ or CD4⁺) expressing the same construct.

In some embodiments, a CD4⁺ T cell comprises a CAR described herein, which CAR comprises an intracellular signaling domain suitable for (e.g., optimized for, e.g., leading to enhanced persistence in) a CD4⁺ T cell, e.g., an ICOS domain. In some embodiments, a CD8⁺ T cell comprises a CAR described herein, which CAR comprises an intracellular signaling domain suitable for (e.g., optimized for, e.g., leading to enhanced persistence of) a CD8⁺ T cell, e.g., a 4-1BB domain, a CD28 domain, or another costimulatory domain other than an ICOS domain. In some embodiments, the CAR described herein comprises an antigen binding domain described herein, e.g., a CAR comprising an antigen binding domain that specifically binds an antigen described herein, e.g., CD123, e.g., a CAR of Table 11A or Table 12A.

In an aspect, described herein is a method of treating a subject, e.g., a subject having cancer. The method includes administering to said subject, an effective amount of:

1) a CD4⁺ T cell comprising a CAR (the CAR^{CD4+})

comprising:

an antigen binding domain, e.g., an antigen binding domain described herein, e.g., an antigen binding domain that specifically binds an antigen described herein, e.g., CD123, e.g., an antigen-binding domain of Table 11A, Table 12A, or Table 12B;

a transmembrane domain; and

an intracellular signaling domain, e.g., a first costimulatory domain, e.g., an ICOS domain; and

2) a CD8⁺ T cell comprising a CAR (the CAR^{CD8+}) comprising:

an antigen binding domain, e.g., an antigen binding domain described herein, e.g., an antigen binding domain that specifically binds an antigen described herein, e.g., CD123, e.g., an antigen-binding domain of Table 11A, Table 12A, or Table 12B;

a transmembrane domain; and

an intracellular signaling domain, e.g., a second co stimulatory domain, e.g., a 4-1BB domain, a CD28 domain, or another costimulatory domain other than an ICOS domain;

wherein the CAR^{CD4+} and the CAR^{CD8+} differ from one another.

Optionally, the method further includes administering:

3) a second CD8+ T cell comprising a CAR (the second CAR^{CD8+}) comprising:

an antigen binding domain, e.g., an antigen binding domain described herein, e.g., an antigen binding domain that specifically binds an antigen described herein, e.g., CD123, e.g., an antigen-binding domain of Table 11A, Table 12A, or Table 12B 9;

a transmembrane domain; and

an intracellular signaling domain, wherein the second CAR^{CD8+} comprises an intracellular signaling domain, e.g., a costimulatory signaling domain, not present on the CAR^{CD8+}, and, optionally, does not comprise an ICOS signaling domain.

### Methods and Compositions for Producing CAR-Expressing Cells

The present disclosure also provides, in certain aspects, a method of making a population of immune effector cells (e.g., T cells or NK cells) that can be engineered to express a CAR (e.g., a CAR described herein), the method comprising: providing a population of immune effector cells; and contacting the immune effector cells with a kinase inhibitor (e.g., a JAK-STAT kinase inhibitor such as ruxolitinib) under conditions sufficient to inhibit a target of the kinase inhibitor (e.g., JAK1, JAK2, JAK3, or TYK2). The method can further comprise contacting, e.g., transducing, the immune effector cells with a nucleic acid encoding a CAR molecule.

In some aspects, the disclosure provides a method of making a CAR-expressing cell (e.g., a CAR-expressing immune effector cell or population of cells), comprising: contacting the cell or population of cells with a kinase inhibitor, e.g., a JAK-STAT kinase inhibitor such as ruxolitinib; and introducing (e.g., transducing) a nucleic acid encoding a CAR molecule into the cell or population of cells under conditions such that the CAR molecule is expressed.

In certain embodiments of the methods of producing CAR-expressing cells, the CAR molecule encoded by the nucleic acid is a CAR molecule that binds an antigen described herein, e.g., tumor antigen described herein, e.g., B cell antigen, e.g., CD123. In embodiments, the method further comprises culturing the cell or cells under conditions that allow the cell or at least a sub-population of the cells to express the CAR molecule. In embodiments, the cell is a T cell or NK cell, or the population of cells includes T cells, NK cells, or both. In embodiments, the method comprises contacting the cell or cells with the JAK-STAT kinase inhibitor (e.g., for 10-20, 20-30, 30-40, 40-60, or 60-120 minutes) and subsequently removing most or all of the kinase inhibitor from the cell or cells. In embodiments, the JAK-STAT kinase inhibitor is added after the cell or cells are harvested or before the cell or cells are stimulated. In embodiments, the JAK-STAT kinase inhibitor is a multi-kinase inhibitor, e.g., that inhibits at least one kinase in the JAK-STAT pathway. In embodiments, the JAK-STAT kinase inhibitor is a JAK1 inhibitor, JAK2 inhibitor, JAK3 inhibitor, or TYK2 inhibitor. In embodiments, the JAK-STAT kinase inhibitor is specific for JAK1, JAK2, JAK3, or TYK2. In embodiments, the JAK-STAT kinase inhibitor is ruxolitinib, AG490, AZD1480, tofacitinib (tasocitinib or CP-690550), CYT387, fedratinib, baricitinib (INCB039110), lestaurtinib (CEP701), pacritinib (SB1518), XL019, gandotinib (LY2784544), BMS911543, fedratinib (SAR302503), decemotinib (V-509), INCB39110, GEN1, GEN2, GLPG0634, NS018, and N-(cyanomethyl)-4-[2-(4-morpholinoanilino)pyrimidin-4-yl]benzamide; or a pharmaceutically acceptable salt thereof. In embodiments, the JAK-STAT kinase inhibitor is ruxolitinib.

In some aspects, the present disclosure also provides a reaction mixture comprising a JAK-STAT kinase inhibitor (e.g., ruxolitinib) and a CAR molecule or a nucleic acid encoding a CAR molecule. In some embodiments, the reaction mixture further comprises a population of immune effector cells.

In some embodiments, one or more of the immune effector cells expresses the CAR molecule or comprises the nucleic acid encoding the CAR molecule. In some embodiments, the JAK-STAT kinase inhibitor is chosen from ruxolitinib, AG490, AZD1480, tofacitinib (tasocitinib or CP-690550), CYT387, fedratinib, baricitinib (INCB039110), lestaurtinib (CEP701), pacritinib (SB1518), XL019, gandotinib (LY2784544), BMS911543, fedratinib (SAR302503), decemotinib (V-509), INCB39110, GEN1, GEN2, GLPG0634, NS018, and N-(cyanomethyl)-4-[2-(4-morpholinoanilino)pyrimidin-4-yl]benzamide; or a pharmaceutically acceptable salt thereof. In embodiments, the reaction mixture comprises cancer cells, e.g., haematological cancer cells. The cancer cells may be, e.g., cells that were harvested from the subject when the immune effector cells were harvested from the subject.

In embodiments, a reaction mixture as described herein further comprises a buffer or other reagent, e.g., a PBS containing solution. In embodiments, the reaction mixture further comprises an agent that activates and/or expands to cells of the population, e.g., an agent that stimulates a CD3/TCR complex associated signal and/or a ligand that stimulates a costimulatory molecule on the surface of the cells. In embodiments, the agent is a bead conjugated with anti-CD3 antibody, or a fragment thereof, and/or anti-CD28 antibody, or a fragment thereof. In embodiments, the reaction mixture further comprises one or more factors for proliferation and/or viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells. In embodiments, the reaction mixture further comprises IL-15 and/or IL-7. In embodiments, a plurality of the cells of the population in the reaction mixture comprise a nucleic acid molecule, e.g., a nucleic acid molecule described herein, that comprises a CAR encoding sequence, e.g., a CD123 CAR encoding sequence, e.g., as described herein. In embodiments, a plurality of the cells of the population in the reaction mixture comprise a vector comprising a nucleic acid sequence encoding a CAR, e.g., a CAR described herein, e.g., a CD123 CAR described herein. In embodiments, the vector is a vector described herein, e.g., a vector selected from the group consisting of a DNA, a RNA, a plasmid, a lentivirus vector, adenoviral vector, or a retrovirus vector. In embodiments, the reaction mixture further comprises a cryoprotectant or stabilizer such as, e.g., a saccharide, an oligosaccharide, a polysaccharide and a polyol (e.g., trehalose, mannitol, sorbitol, lactose, sucrose, glucose and dextran), salts and crown ethers. In one embodiment, the cryoprotectant is dextran.

In some embodiments, the method of making described herein further comprises contacting the population of immune effector cells with a nucleic acid encoding a telomerase subunit, e.g., hTERT. The the nucleic acid encoding the telomerase subunit can be DNA.

In some embodiments, the method of making discosed herein further comprises culturing the population of immune effector cells in serum comprising 2% hAB serum.

### Therapeutic Application

In accordance with any method described herein, in embodiments, a subject has a cancer, e.g., hematological cancer or a solid cancer. In embodiments, a composition described herein can be used to treat a cancer described herein. In embodiments, a JAK-STAT inhibitor, e.g., ruxolitinib, is used in combination with a CAR-expressing cell (e.g., CD123 CAR expressing cell) to treat a cancer.

The present invention provides, among other things, compositions and methods for treating a disease associated with expression of an antigen (e.g., CD123) or condition associated with cells which express the antigen (e.g., CD123) including, e.g., a proliferative disease such as a cancer or malignancy or a precancerous condition such as a myelodysplasia, a myelodysplastic syndrome or a preleukemia; or a noncancer related indication associated with cells which express the antigen (e.g., CD123). In one aspect, a cancer associated with expression of an antigen (e.g., CD123) is a hematological cancer. In one aspect, a hematological cancer includes but is not limited to AML, myelodysplastic syndrome, ALL, chronic myeloid leukemia, blastic plasmacytoid dendritic cell neoplasm, myeloproliferative neoplasms, Hodgkin lymphoma, and the like. In embodiments, disease associated with expression of CD123 expression includes, but are not limited to, e.g., atypical and/or non-classical cancers, malignancies, precancerous conditions or proliferative diseases associated with expression of CD123. Non-cancer related indications associated with expression of an antigen (e.g., CD123) may also be included.

In one aspect, the invention provides methods for treating a disease associated with expression of antigen (e.g., CD123 expression). In one aspect, the invention provides methods for treating a disease wherein part of the tumor is negative for the antigen (e.g., CD123) and part of the tumor is positive for the antigen (e.g., CD123). For example, the CAR described herein is useful for treating subjects that have undergone treatment for a disease associated with elevated expression of the antigen (e.g., CD123), wherein the subject that has undergone treatment for elevated levels of the antigen (e.g., CD123) exhibits a disease associated with elevated levels of the antigen (e.g., CD123). In embodiments, the CAR is useful for treating subjects that have undergone treatment for a disease associated with expression of the antigen (e.g., CD123), wherein the subject that has undergone treatment related to expression of the antigen (e.g., CD123) exhibits a disease associated with expression of the antigen (e.g., CD123).

In one aspect, provided herein is a method of inhibiting growth of an antigen-expressing (e.g., CD123-expressing) tumor cell, comprising contacting the tumor cell with a CAR-expressing cell (e.g., CD123 CAR-expressing cell (e.g., CD123 CART or CD123 CAR-expressing NK cell)) such that the CAR-expressing cell is activated in response to the antigen and targets the cancer cell, wherein the growth of the tumor is inhibited. The method can comprise administration of a JAK-STAT inhibitor or BTK inhibitor.

In one aspect, the invention pertains to a method of treating cancer in a subject. The method comprises administering to the subject a CAR-expressing cell (e.g., CD123 CAR-expressing cell (e.g., CD123 CART or CD123 CAR-expressing NK cell)) described herein such that the cancer is treated in the subject. The cellular therapy is provided in combination with a JAK-STAT inhibitor or BTK inhibitor. An example of a cancer that is treatable by a CD123 CAR-expressing cell (e.g., CD123 CART or CD123 CAR-expressing NK cell) is a cancer associated with expression of CD123. An example of a cancer that is treatable by a CD123 CAR-expressing cell (e.g., CD123 CART or CD123 CAR-expressing NK cell) includes but is not limited to AML, Hodgkin lymphoma, myelodysplastic syndrome, chronic myeloid leukemia and other myeloproliferative neoplasms, or Blastic plasmacytoid dendritic cell neoplasm, and the like.

The disclosure includes a type of cellular therapy where immune effector cells, e.g., T cells or NK cells, are genetically modified to express a chimeric antigen receptor (CAR) and the CAR-expressing cell (e.g., CD123 CAR-expressing cell (e.g., CD123 CART or CD123 CAR-expressing NK cell)) is infused to a recipient in need thereof. The infused cell is able to kill tumor cells in the recipient. The cellular therapy is provided in combination with a JAK-STAT inhibitor or BTK inhibitor. Unlike antibody therapies, CAR-modified immune effector cells, e.g., the CAR-modified T cells or CAR-modified NK cells) are able to replicate in vivo resulting in long-term persistence that can lead to sustained tumor control. In various aspects, the immune effector cells, e.g., T cells or NK cells, administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the immune effector cell, e.g., T cell or NK cell, to the patient.

The invention also includes a type of cellular therapy where immune effector cells, e.g., T cells or NK cells, are modified, e.g., by in vitro transcribed RNA, to transiently express a chimeric antigen receptor (CAR) and the CAR-expressing cell, e.g., CAR T cell or CAR NK cell) is infused to a recipient in need thereof. The cellular therapy is provided in combination with a JAK-STAT inhibitor or BTK inhibitor. The infused cell is able to kill tumor cells in the recipient. Thus, in various aspects, the immune effector cells, e.g., T cells or NK cells, administered to the patient, is present for less than one month, e.g., three weeks, two weeks, one week, after administration of the immune effector cell, e.g., T cell or NK cell, to the patient.

Without wishing to be bound by any particular theory, the anti-tumor immunity response elicited by the CAR-modified immune effector cells, e.g., T cells or NK cells, may be an active or a passive immune response, or alternatively may be due to a direct vs indirect immune response. In one aspect, the CAR transduced immune effector cells, e.g., T cells or NK cells, exhibit specific proinflammatory cytokine secretion and potent cytolytic activity in response to human cancer cells expressing CD123, resist soluble CD123 inhibition, mediate bystander killing and mediate regression of an established human tumor. For example, antigen-less tumor cells within a heterogeneous field of CD123-expressing tumor may be susceptible to indirect destruction by CD123-redirected immune effector cell, e.g., T cells or NK cells that has previously reacted against adjacent antigen-positive cancer cells.

In one aspect, the fully-human CAR-modified immune effector cells (e.g., T cells or NK cells) of the invention may be a type of vaccine for ex vivo immunization and/or in vivo therapy in a mammal. In one aspect, the mammal is a human.

With respect to ex vivo immunization, at least one of the following occurs in vitro prior to administering the cell, e.g., T cell or NK cell, into a mammal: i) expansion of the cells, ii) introducing a nucleic acid encoding a CAR to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised. In particular, the CAR-modified immune effector cells (e.g., T cells or NK cells) of the invention are used in the treatment of diseases, disorders and conditions described herein, e.g., disorders or conditions associated with expression of an antigen described herein, e.g., CD123 or CD19. In certain aspects, the cells of the invention are used in the treatment of patients at risk for developing diseases, disorders and conditions described herein, e.g., disorders or conditions associated with expression of an antigen described herein, e.g., CD123 or CD19. Thus, the present invention provides methods for the treatment or prevention of diseases, disorders and conditions described herein, e.g., disorders or conditions associated with expression of an antigen described herein, e.g., CD123 or CD19, comprising administering to a subject in need thereof, a therapeutically effective amount of the CAR-modified immune effector cells (e.g., T cells or NK cells) described herein in combination with a JAK-STAT inhibitor or a BTK inhibitor.

In one aspect, the CAR-expressing cells (CART cells or CAR-expressing NK cells) of the inventions may be used to treat a proliferative disease such as a cancer or malignancy or is a precancerous condition such as a myelodysplasia, a myelodysplastic syndrome or a preleukemia. In one aspect, the cancer is a hematological cancer preleukemia, a hyperproliferative disorder, a hyperplasia or a dysplasia, which is characterized by abnormal growth of cells.

In one aspect, the CAR-expressing cells (CART cells or CAR-expressing NK cells) of the invention are used to treat a cancer, wherein the cancer is a hematological cancer.

Hematological cancer conditions are the types of cancer such as leukemia and malignant lymphoproliferative conditions that affect blood, bone marrow and the lymphatic system.

In one aspect, the compositions and CAR-expressing cells (CART cells or CAR-expressing NK cells) of the present invention are particularly useful for treating myeloid leukemias, AML and its subtypes, chronic myeloid leukemia (CML), myelodysplastic syndrome (MDS), myeloproliferative neoplasms (MPN), histiocytic disorders, and mast cell disorders.

Also provided herein are methods for inhibiting the proliferation of or reducing an antigen-expressing (e.g., CD123-expressing or CD19-expressing) cell population, the methods comprising contacting a population of cells comprising an antigen-expressing (e.g., CD123-expressing or CD19-expressing) cell with a CAR-expressing cell (e.g., CD123 CAR-expressing cell or CD19 CAR-expressing cell) that binds to the antigen-expressing (e.g., CD123-expressing or CD19-expressing) cell. In a specific aspect, the present invention provides methods for inhibiting the proliferation of or reducing the population of cancer cells expressing the antigen (e.g., CD123 or CD19), the methods comprising contacting the antigen-expressing (e.g., CD123-expressing or CD19-expressing) cancer cell population with a CAR-expressing cell (e.g., CD123 CAR-expressing cell or CD19 CAR-expressing cell) that binds to the antigen-expressing (e.g., CD123-expressing or CD19-expressing). In one aspect, the present invention provides methods for inhibiting the proliferation or reducing the population of cancer cells expressing an antigen (e.g., CD123), the methods comprising contacting the antigen-expressing (e.g., CD123-expressing or CD19-expressing) cancer cell population with a CAR-expressing cell (e.g., CD123 CAR-expressing cell or CD19 CAR-expressing cell) that binds to the antigen-expressing cell. In certain aspects, the CAR-expressing cell (e.g., CD123-expressing or CD19-expressing cell) reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for myeloid leukemia or another cancer associated with the antigen-expressing cells (e.g., CD123-expressing cells or CD19-expressing cells) relative to a negative control. In one aspect, the subject is a human.

The present invention also provides methods for preventing, treating and/or managing a disease associated with antigen expressing cell (e.g., CD123-expressing cells or CD19-expressing cells) (e.g., a hematologic cancer or atypical cancer expessing the antigen, e.g., CD123 or CD19), the methods comprising administering to a subject in need a CAR-expressing cell (e.g., CD123 CAR-expressing or CD19 CAR-expressing cell) that binds to the antigen-expressing cell. In one aspect, the subject is a human. Non-limiting examples of disorders associated with various antigens, e.g, CD123-expressing cells, include autoimmune disorders (such as lupus), inflammatory disorders (such as allergies and asthma) and cancers (such as hematological cancers or atypical cancers expessing the antigen e,g., CD123).

The present invention also provides methods for preventing, treating and/or managing a disease associated with antigen-expressing cells (e.g., CD123-expressing cells or CD19-expressing cells), the methods comprising administering to a subject in need a CAR-expressing cell (e.g., CD123 CAR-expressing cell or CD19 CAR-expressing cell) that binds to the antigen-expressing cell. In one aspect, the subject is a human.

The present invention provides methods for preventing relapse of cancer associated with antigen-expressing cells (e.g., CD123-expressing or CD19-expressing cells), the methods comprising administering to a subject in need thereof a CAR-expressing cell (e.g., CD123 CAR-expressing cell or CD19 CAR-expressing cell) of the invention that binds to the antigen-expressing cell, in combination with a JAK-STAT inhibitor or BTK inhibitor. In one aspect, the methods comprise administering to the subject in need thereof an effective amount of a CAR-expressing cell (e.g., CD123 CAR-expressing cell or CD19 CAR-expressing cell) described herein that binds to the antigen-expressing cell in combination with an effective amount of another therapy (e.g., JAK-STAT inhibitor or BTK inhibitor).

Bone Marrow Ablation

In one aspect, the present invention provides compositions and methods for bone marrow ablation. For example, in one aspect, the invention provides compositions and methods for eradication of at least a portion of existing bone marrow in a subject. It is described herein that, in certain instances, the CART123 cells comprising a CD123 CAR of the present invention eradicates CD123 positive bone marrow myeloid progenitor cells.

In one aspect, the invention provides a method of bone marrow ablation comprising administering a CAR-expressing cell (e.g., CD123 CART cell or CD123 CAR-expressing NK cell) of the invention to a subject in need of bone marrow ablation, e.g., in combination with a JAK-STAT inhibitor. For example, the present method may be used to eradicate some or all of the existing bone marrow of a subject having a disease or disorder in which bone marrow transplantation or bone marrow reconditioning is a beneficial treatment strategy. In one aspect, the bone marrow ablation method of the invention, comprising the administration of a CAR-expressing cell (e.g., CD123 CART cell or CD123 CAR-expressing NK cell) described elsewhere herein, is performed in a subject prior to bone marrow transplantation. Thus, in one aspect, the method of the invention provides a cellular conditioning regimen prior to bone marrow or stem cell transplantation. In one aspect, bone marrow transplantation comprises transplantation of a stem cell. The bone marrow transplantation may comprise transplantation of autologous or allogeneic cells.

The present invention provides a method of treating a disease or disorder comprising administering a CAR-expressing cell (e.g., CD123 CART cell or CD123 CAR-expressing NK cell, or a CD19 CART cell or CD19 CAR-expressing NK cell) to eradicate at least a portion of existing bone marrow. The method may be used as at least a portion of a treatment regimen for treating any disease or disorder where bone marrow transplantation is beneficial. That is, the present method may be used in any subject in need of a bone marrow transplant. In one aspect, bone marrow ablation comprising administration of a CAR-expressing cell (e.g., CD123 CART cell or CD123 CAR-expressing NK cell) is useful in the treatment of AML. In certain aspects, bone marrow ablation by way of the present method is useful in treating a hematological cancer, a solid tumor, a hematologic disease, a metabolic disorder, HIV, HTLV, a lysosomal storage disorder, and an immunodeficiency.

Compositions and methods disclosed herein may be used to eradicate at least a portion of existing bone marrow to treat hematological cancers including, but not limited to, leukemia, lymphoma, myeloma, ALL, AML, CLL, CML, Hodgkin's lymphoma, Non-Hodgkin's lymphoma, and multiple myeloma.

Compositions and methods disclosed herein may be used to treat hematologic diseases including, but not limited to myelodysplasia, anemia, paroxysmal nocturnal hemoglobinuria, aplastic anemia, acquired pure red cell anemia, Diamon-Blackfan anemia, Fanconi anemia, cytopenia, amegakaryotic thrombocytopenia, myeloproliferative disorders, polycythemia vera, essential thrombocytosis, myelofibrosis, hemoglobinopathies, sickle cell disease, β thalassemia major, among others.

Compositions and methods disclosed herein may be used to treat lysosomal storage disorders including, but not limited to lipidoses, sphigolipodeses, leukodystrophies, mucopolysaccharidoses, glycoproteinoses, infantile neuronal ceroid lipofuscinosis, Jansky-Bielschowsky disease, Niemann-Pick disease, Gaucher disease, adrenoleukodystrophy, metachromatic leukodystrophy, Krabbe disease, Hurler syndrome, Scheie syndrome, Hurler-Scheie syndrome, hunter syndrome, Sanfilippo syndrome, Morquio syndrome, Maroteaux-Lamy syndrome, Sly syndrome, mucolipidosis, fucolipidosis, aspartylglucosaminuria, alpha-mannosidoses, and Wolman disease.

Compositions and methods disclosed herein may be used to treat immunodeficiencies including, but not limited to, T-cell deficiencies, combined T-cell and B-cell deficiencies, phagocyte disorders, immune dysregulation diseases, innate immune deficiencies, ataxia telangiectasia, DiGeorge syndrome, severe combined immunodeficiency (SCID), Wiskott-Aldrich syndrome, Kostmann syndrome, Shwachman-Diamond syndrome, Griscelli syndrome, and NF-Kappa-B Essential Modulator (NEMO) deficiency.

In one aspect, the present invention provides a method of treating cancer comprising bone marrow conditioning, where at least a portion of bone marrow of the subject is eradicated by the CAR-expressing cell (e.g., CD123 CART cell or CD123 CAR-expressing NK cell, or CD19 CART cell or CD19 CAR-expressing NK cell) of the invention. For example, in certain instances, the bone marrow of the subject comprises a malignant precursor cell that can be targeted and eliminated by the activity of the CAR-expressing cell. In one aspect, a bone marrow conditioning therapy comprises administering a bone marrow or stem cell transplant to the subject following the eradication of native bone marrow. In one aspect, the bone marrow reconditioning therapy is combined with one or more other anti-cancer therapies, including, but not limited to anti-tumor CAR therapies, chemotherapy, radiation, and the like.

In one aspect, eradication of the administered CAR-expressing cell (e.g., CD123 CART cell or CD123 CAR-expressing NK cell, or CD19 CART cell or CD19 CAR-expressing NK cell) may be required prior to infusion of bone marrow or stem cell transplant. Eradication of the CAR-expressing cell may be accomplished using any suitable strategy or treatment, including, but not limited to, use of a suicide gene, limited CAR persistence using RNA encoded CARs, or anti-T cell modalities including antibodies or chemotherapy.

### Hematologic Cancers

Hematological cancer conditions are the types of cancer such as leukemia, lymphoma and malignant lymphoproliferative conditions that affect blood, bone marrow and the lymphatic system.

In one embodiment, the hematologic cancer is leukemia. In one embodiment, the cancer is selected from the group consisting of one or more acute leukemias including but not limited to B-cell acute lymphoid leukemia (BALL), T-cell acute lymphoid leukemia (TALL), small lymphocytic leukemia (SLL), acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to mantle cell lymphoma (MCL), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

In an aspect, the invention pertains to a method of treating a mammal having a hematological cancer, comprising administering to the mammal an effective amount of the cells expressing a CAR molecule (e.g., CD123 CAR molecule or a CD19 CAR molecule), e.g., a CAR molecule (e.g., CD123 CAR or CD19 CAR) and a JAK-STAT inhibitor or BTK inhibitor.

In one aspect, the compositions and CART cells or CAR expressing NK cells of the present invention are particularly useful for treating B cell malignancies, such as non-Hodgkin lymphomas, e.g., DLBCL, Follicular lymphoma, or CLL. In some cases, the compositions and CART cells or CAR expressing NK cells of the present invention are particularly useful for treating AML.

Non-Hodgkin lymphoma (NHL) is a group of cancers of lymphocytes, formed from either B or T cells. NHLs occur at any age and are often characterized by lymph nodes that are larger than normal, weight loss, and fever. Different types of NHLs are categorized as aggressive (fast-growing) and indolent (slow-growing) types. B-cell non-Hodgkin lymphomas include Burkitt lymphoma, chronic lymphocytic leukemia/small lymphocytic lymphoma (CLL/SLL), diffuse large B-cell lymphoma (DLBCL), follicular lymphoma, immunoblastic large cell lymphoma, precursor B-lymphoblastic lymphoma, and mantle cell lymphoma. Examples of T-cell non-Hodgkin lymphomas include mycosis fungoides, anaplastic large cell lymphoma, and precursor T-lymphoblastic lymphoma. Lymphomas that occur after bone marrow or stem cell transplantation are typically B-cell non-Hodgkin lymphomas. See, e.g., Maloney. NEJM. 366.21(2012):2008-16.

Diffuse large B-cell lymphoma (DLBCL) is a form of NHL that develops from B cells. DLBCL is an aggressive lymphoma that can arise in lymph nodes or outside of the lymphatic system, e.g., in the gastrointestinal tract, testes, thyroid, skin, breast, bone, or brain. Three variants of cellular morphology are commonly observed in DLBCL: centroblastic, immunoblastic, and anaplastic. Centroblastic morphology is most common and has the appearance of medium-to-large-sized lymphocytes with minimal cytoplasm. There are several subtypes of DLBCL. For example, primary central nervous system lymphoma is a type of DLBCL that only affects the brain is called and is treated differently than DLBCL that affects areas outside of the brain. Another type of DLBCL is primary mediastinal B-cell lymphoma, which often occurs in younger patients and grows rapidly in the chest. Symptoms of DLBCL include a painless rapid swelling in the neck, armpit, or groin, which is caused by enlarged lymph nodes. For some subjects, the swelling may be painful. Other symptoms of DLBCL include night sweats, unexplained fevers, and weight loss. Although most patients with DLBCL are adults, this disease sometimes occurs in children. Treatment for DLBCL includes chemotherapy (e.g., cyclophosphamide, doxorubicin, vincristine, prednisone, etoposide), antibodies (e.g., Rituxan), radiation, or stem cell transplants.

Follicular lymphoma a type of non-Hodgkin lymphoma and is a lymphoma of follicle center B-cells (centrocytes and centroblasts), which has at least a partially follicular pattern. Follicular lymphoma cells express the B-cell markers CD10, CD19, CD20, and CD22. Follicular lymphoma cells are commonly negative for CD5. Morphologically, a follicular lymphoma tumor is made up of follicles containing a mixture of centrocytes (also called cleaved follicle center cells or small cells) and centroblasts (also called large noncleaved follicle center cells or large cells). The follicles are surrounded by non-malignant cells, mostly T-cells. The follicles contain predominantly centrocytes with a minority of centroblasts. The World Health Organization (WHO) morphologically grades the disease as follows: grade 1 (<5 centroblasts per high-power field (hpf); grade 2 (6-15 centroblasts/hpf); grade 3 (>15 centroblasts/hpf). Grade 3 is further subdivided into the following grades: grade 3A (centrocytes still present); grade 3B (the follicles consist almost entirely of centroblasts). Treatment of follicular lymphoma includes chemotherapy, e.g., alkyating agents, nucleoside analogs, anthracycline-containing regimens, e.g., a combination therapy called CHOP-cyclophosphamide, doxorubicin, vincristine, prednisone/prednisolone, antibodies (e.g., rituximab), radioimmunotherapy, and hematopoietic stem cell transplantation.

CLL is a B-cell malignancy characterized by neoplastic cell proliferation and accumulation in bone morrow, blood, lymph nodes, and the spleen. The median age at time of diagnosis of CLL is about 65 years. Current treatments include chemotherapy, radiation therapy, biological therapy, or bone marrow transplantation. Sometimes symptoms are treated surgically (e.g., splenectomy removal of enlarged spleen) or by radiation therapy (e.g., de-bulking swollen lymph nodes). Chemotherapeutic agents to treat CLL include, e.g., fludarabine, 2-chlorodeoxyadenosine (cladribine), chlorambucil, vincristine, pentostatin, cyclophosphamide, alemtuzumab (Campath-1H), doxorubicin, and prednisone. Biological therapy for CLL includes antibodies, e.g., alemtuzumab, rituximab, and ofatumumab; as well as tyrosine kinase inhibitor therapies. A number of criteria can be used to classify stage of CLL, e.g., the Rai or Binet system. The Rai system describes CLL has having five stages: stage 0 where only lymphocytosis is present; stage I where lymphadenopathy is present; stage II where splenomegaly, lymphadenopathy, or both are present; stage III where anemia, organomegaly, or both are present (progression is defined by weight loss, fatigue, fever, massive organomegaly, and a rapidly increasing lymphocyte count); and stage IV where anemia, thrombocytopenia, organomegaly, or a combination thereof are present. Under the Binet staging system, there are three categories: stage A where lymphocytosis is present and less than three lymph nodes are enlarged (this stage is inclusive of all Rai stage 0 patients, one-half of Rai stage I patients, and one-third of Rai stage II patients); stage B where three or more lymph nodes are involved; and stage C wherein anemia or thrombocytopenia, or both are present. These classification systems can be combined with measurements of mutation of the immunoglobulin genes to provide a more accurate characterization of the state of the disease. The presence of mutated immunoglobulin genes correlates to improved prognosis.

In another embodiment, the CAR expressing cells of the present invention are used to treat cancers or leukemias, e.g., with leukemia stem cells. For example, the leukemia stem cells are CD34⁺/CD38⁻ leukemia cells.

In one aspect, the compositions and CAR-expressing cells (CART cells or CAR-expressing NK cells) of the present invention are particularly useful for treating myeloid leukemias, AML and its subtypes, chronic myeloid leukemia (CML), myelodysplastic syndrome (MDS), myeloproliferative neoplasms (MPN), histiocytic disorders, and mast cell disorders.

Leukemia can be classified as acute leukemia and chronic leukemia. Acute leukemia can be further classified as acute myelogenous leukemia (AML) and acute lymphoid leukemia (ALL). Chronic leukemia includes chronic myelogenous leukemia (CML) and chronic lymphoid leukemia (CLL). Other related conditions include myelodysplastic syndromes (MDS, formerly known as “preleukemia”) which are a diverse collection of hematological conditions united by ineffective production (or dysplasia) of myeloid blood cells and risk of transformation to AML.

Lymphoma is a group of blood cell tumors that develop from lymphocytes. Exemplary lymphomas include non-Hodgkin lymphoma and Hodgkin lymphoma.

In AML, malignant transformation and uncontrolled proliferation of an abnormally differentiated, long-lived myeloid progenitor cell results in high circulating numbers of immature blood forms and replacement of normal marrow by malignant cells. Symptoms include fatigue, pallor, easy bruising and bleeding, fever, and infection; symptoms of leukemic infiltration are present in only about 5% of patients (often as skin manifestations). Examination of peripheral blood smear and bone marrow is diagnostic. Existing treatment includes induction chemotherapy to achieve remission and post-remission chemotherapy (with or without stem cell transplantation) to avoid relapse.

AML has a number of subtypes that are distinguished from each other by morphology, immunophenotype, and cytochemistry. Five classes are described, based on predominant cell type, including myeloid, myeloid-monocytic, monocytic, erythroid, and megakaryocytic.

Remission induction rates range from 50 to 85%. Long-term disease-free survival reportedly occurs in 20 to 40% of patients and increases to 40 to 50% in younger patients treated with stem cell transplantation.

Prognostic factors help determine treatment protocol and intensity; patients with strongly negative prognostic features are usually given more intense forms of therapy, because the potential benefits are thought to justify the increased treatment toxicity. The most important prognostic factor is the leukemia cell karyotype; favorable karyotypes include t(15;17), t(8;21), and inv16 (p13;q22). Negative factors include increasing age, a preceding myelodysplastic phase, secondary leukemia, high WBC count, and absence of Auer rods.

Initial therapy attempts to induce remission and differs most from ALL in that AML responds to fewer drugs. The basic induction regimen includes cytarabine by continuous IV infusion or high doses for 5 to 7 days; daunorubicin or idarubicin is given IV for 3 days during this time. Some regimens include 6-thioguanine, etoposide, vincristine, and prednisone, but their contribution is unclear. Treatment usually results in significant myelosuppression, with infection or bleeding; there is significant latency before marrow recovery. During this time, meticulous preventive and supportive care is vital.

Chronic myelogenous (or myeloid) leukemia (CML) is also known as chronic granulocytic leukemia, and is characterized as a cancer of the white blood cells. Common treatment regimens for CML include Bcr-Abl tyrosine kinase inhibitors, imatinib (Gleevec®), dasatinib and nilotinib. Bcr-Abl tyrosine kinase inhibitors are specifically useful for CML patients with the Philadelphia chromosome translocation.

Myelodysplastic syndromes (MDS) are hematological medical conditions characterized by disorderly and ineffective hematopoiesis, or blood production. Thus, the number and quality of blood-forming cells decline irreversibly. Some patients with MDS can develop severe anemia, while others are asymptomatic. The classification scheme for MDS is known in the art, with criteria designating the ratio or frequency of particular blood cell types, e.g., myeloblasts, monocytes, and red cell precursors. MDS includes refractory anemia, refractory anemia with ring sideroblasts, refractory anemia with excess blasts, refractory anemia with excess blasts in transformation, chronic myelomonocytic leukemia (CML).

Treatments for MDS vary with the severity of the symptoms. Aggressive forms of treatment for patients experiencing severe symptoms include bone marrow transplants and supportive care with blood product support (e.g., blood transfusions) and hematopoietic growth factors (e.g., erythropoietin). Other agents are frequently used to treat MDS: 5-azacytidine, decitabine, and lenalidomide. In some cases, iron chelators deferoxamine (Desferal®) and deferasirox (Exjade®) may also be administered.

### Solid Cancers

Exemplary solid cancers include but are not limited to: uterine cancer, colon cancer, ovarian cancer, rectal cancer, skin cancer, stomach cancer, lung cancer, non-small cell carcinoma of the lung, breast cancer, cancer of the small intestine, testicular cancer, cancer of the anal region, cancer of the endocrine system, cancer of the thyroid gland, cancer of the parathyroid gland, rectal cancer, renal-cell carcinoma, liver cancer, cancer of the esophagus, melanoma, cutaneous or intraocular malignant melanoma, uterine cancer, brain cancer, brain stem glioma, pituitary adenoma, Kaposi's sarcoma, cancer of the adrenal gland, bone cancer, pancreatic cancer, cancer of the head or neck, epidermoid cancer, carcinoma of the endometrium, carcinoma of the vagina, cervical cancer, sarcoma, uterine cancer, stomach cancer, esophageal cancer, colorectal cancer, liver cancer, prostate cancer, carcinoma of the cervix squamous cell cancer, carcinoma of the fallopian tubes, sarcoma of soft tissue, cancer of the urethra, carcinoma of the vulva, cancer of the kidney or ureter, carcinoma of the renal pelvis, spinal axis tumor, cancer of the penis, cancer of the bladder, neoplasm of the central nervous system (CNS), primary CNS lymphoma, tumor angiogenesis, metastatic lesions of said cancers, and/or combinations thereof

### B Cell Cancers

Many patients with B cell malignancies are incurable with standard therapy. In addition, traditional treatment options often have serious side effects. Attempts have been made in cancer immunotherapy, however, several obstacles render this a very difficult goal to achieve clinical effectiveness. Although hundreds of so-called tumor antigens have been identified, these are generally derived from self and thus are poorly immunogenic. Furthermore, tumors use several mechanisms to render themselves hostile to the initiation and propagation of immune attack.

Recent developments using chimeric antigen receptor (CAR) modified autologous T cell (CART) therapy, which relies on redirecting T cells to a suitable cell-surface molecule on cancer cells such as B cell malignancies, show promising results in harnessing the power of the immune system to treat B cell malignancies and other cancers (see, e.g., Sadelain et al., Cancer Discovery 3:388-398 (2013)). The clinical results of the murine derived CART19 (i.e., “CTL019”) have shown promise in establishing complete remissions in patients suffering with CLL as well as in childhood ALL (see, e.g., Kalos et al., Sci Transl Med 3:95ra73 (2011), Porter et al., NEJM 365:725-733 (2011), Grupp et al., NEJM 368:1509-1518 (2013)). Besides the ability for the chimeric antigen receptor on the genetically modified T cells to recognize and destroy the targeted cells, a successful therapeutic T cell therapy needs to have the ability to proliferate and persist over time, in order to survey for leukemic relapse. The variable quality of T cells, resulting from anergy, suppression, or exhaustion, will have effects on CAR-transformed T cells' performance, over which skilled practitioners have limited control at this time. To be effective, CAR transformed patient T cells need to persist and maintain the ability to proliferate in response to the cognate antigen. It has been shown that ALL patient T cells perform can do this with CART19 comprising a murine scFv (see, e.g., Grupp et al., NEJM 368:1509-1518 (2013)).

In embodiments, a B cell inhibitor comprises one or more inhibitors of CD10, CD19, CD20, CD22, CD34, CD123, FLT-3, ROR1, CD79b, CD179b, or CD79a.

### Methods of Treating or Preventing CRS

In yet another aspect, provided herein is a method of treating or preventing CRS associated with administration of a cell, e.g., a population of cells, expressing a CAR in a subject.

In yet another aspect, provided herein is a method of treating or preventing CRS associated with administration of a T cell inhibitor therapy, e.g., a CD19-inhibiting or depleting therapy, e.g., a therapy that includes a CD19 inhibitor. In embodiments, the CD19-inhibiting or depleting therapy is associated with CRS.

In some embodiments, the method of treating or preventing CRS comprises administering to the subject a JAK/STAT innibitor, in combination as described herein.

In other embodiments, the method of treating or preventing CRS comprises administering to the subject a BTK innibitor, in combination as described herein.

In yet other embodiments, the method of treating or preventing CRS comprises administering to the subject an IL-6 inhibitor (e.g., an anti-IL6 receptor inhibitor, e.g., tocilizumab) prior to, simultaneously with, or within 1 day (e.g, within 24 hours, 12 hours, 6 hours, 5, hours, 4 hours, 3 hours, 2 hours, 1 hour or less) of, administration of a dose (e.g., a first dose) of said cell, e.g., said population of cells, expressing a CAR, or said therapy. In embodiments, the IL-6 inhibitor (e.g., tocilizumab) is administered upon (e.g., within 1 hour, 30 minutes, 20 minutes, 15 minutes or less) a first sign of a symptom of CRS (e.g., a fever, e.g., characterized by a temperature of at least 38° C. (e.g., at least 38.5° C.), e.g., for two successive measurements in 24 hours (e.g., at least 4, 5, 6, 7, 8 hours, or more, apart)) in the subject.

In other embodiments, the therapy is a CD19-inhibiting or depleting therapy, e.g., a therapy that includes a CD19 inhibitor. In embodiments, the CD19-inhibiting or depleting therapy is associated with CRS. In some embodiments, the CD19 inhibitor is a CD19 antibody, e.g., a CD19 bispecific antibody (e.g., a bispecific T cell engager that targets CD19, e.g., blinatumomab). In some embodiments, the bispecific T cell engager antibody molecule is an antibody molecule described in Bargou et al., “Tumor regression in cancer patients by very low doses of a T cell-engaging antibody.” Science. 2008 Aug. 15; 321(5891):974-7. doi: 10.1126/science.1158545.

In some embodiments, the therapy includes a CD19 CAR-expressing cell, e.g., a CD19 CART cell, or an anti-CD19 antibody (e.g., an anti-CD19 mono- or bispecific antibody) or a fragment or conjugate thereof. In one embodiment, the anti-CD19 antibody is a humanized antigen binding domain as described in WO2014/153270 (e.g., Table 3 of WO2014/153270) incorporated herein by reference, or a conjugate thereof. Other exemplary anti-CD19 antibodies or fragments or conjugates thereof, include but are not limited to, blinatumomab, SAR3419 (Sanofi), MEDI-551 (Medlmmune LLC), Combotox, DT2219ARL (Masonic Cancer Center), MOR-208 (also called XmAb-5574; MorphoSys), XmAb-5871 (Xencor), MDX-1342 (Bristol-Myers Squibb), SGN-CD19A (Seattle Genetics), and AFM11 (Affimed Therapeutics). See, e.g., Hammer. MAbs. 4.5(2012): 571-77. Blinatomomab is a bispecific antibody comprised of two scFvs—one that binds to CD19 and one that binds to CD3. Blinatomomab directs T cells to attack cancer cells. See, e.g., Hammer et al.; Clinical Trial Identifier No. NCT00274742 and NCT01209286. MEDI-551 is a humanized anti-CD19 antibody with a Fc engineered to have enhanced antibody-dependent cell-mediated cytotoxicity (ADCC). See, e.g., Hammer et al.; and Clinical Trial Identifier No. NCT01957579. Combotox is a mixture of immunotoxins that bind to CD19 and CD22. The immunotoxins are made up of scFv antibody fragments fused to a deglycosylated ricin A chain. See, e.g., Hammer et al.; and Herrera et al. J. Pediatr. Hematol. Oncol. 31.12(2009):936-41; Schindler et al. Br. J. Haematol. 154.4(2011):471-6. DT2219ARL is a bispecific immunotoxin targeting CD19 and CD22, comprising two scFvs and a truncated diphtheria toxin. See, e.g., Hammer et al.; and Clinical Trial Identifier No. NCT00889408. SGN-CD19A is an antibody-drug conjugate (ADC) comprised of an anti-CD19 humanized monoclonal antibody linked to a synthetic cytotoxic cell-killing agent, monomethyl auristatin F (MMAF). See, e.g., Hammer et al.; and Clinical Trial Identifier Nos. NCT01786096 and NCT01786135. SAR3419 is an anti-CD19 antibody-drug conjugate (ADC) comprising an anti-CD19 humanized monoclonal antibody conjugated to a maytansine derivative via a cleavable linker. See, e.g., Younes et al. J. Clin. Oncol. 30.2(2012): 2776-82; Hammer et al.; Clinical Trial Identifier No. NCT00549185; and Blanc et al. Clin Cancer Res. 2011; 17:6448-58. XmAb-5871 is an Fc-engineered, humanized anti-CD19 antibody. See, e.g., Hammer et al. MDX-1342 is a human Fc-engineered anti-CD19 antibody with enhanced ADCC. See, e.g., Hammer et al. In embodiments, the antibody molecule is a bispecific anti-CD19 and anti-CD3 molecule. For instance, AFM11 is a bispecific antibody that targets CD19 and CD3. See, e.g., Hammer et al.; and Clinical Trial Identifier No. NCT02106091. In some embodiments, an anti-CD19 antibody described herein is conjugated or otherwise bound to a therapeutic agent, e.g., a chemotherapeutic agent, peptide vaccine (such as that described in Izumoto et al. 2008 J Neurosurg 108:963-971), immunosuppressive agent, or immunoablative agent, e.g., cyclosporin, azathioprine, methotrexate, mycophenolate, FK506, CAMPATH, anti-CD3 antibody, cytoxin, fludarabine, rapamycin, mycophenolic acid, steroid, FR901228, or cytokine.

### Combination Therapies

A CAR-expressing cell described herein may be used in combination with a JAK-STAT inhibitor or a BTK inhibitor. The combination of the CAR-expressing cell and the JAK-STAT inhibitor or a BTK inhibitor can be used in further combination with other known agents and therapies (additional therapeutic agent). Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed. The JAK-STAT inhibitor or BTK inhibitor can be administered before, concurrently with, or after the CAR-expressing cell or the additional agent.

The CAR therapy and/or other therapeutic agents, procedures or modalities can be administered during periods of active disorder, or during a period of remission or less active disease. The CAR therapy can be administered before the other treatment, concurrently with the treatment, post-treatment, or during remission of the disorder.

When administered in combination, the CAR therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the CAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

### JAK-STAT Signaling Pathway and Inhibitors

The JAK-STAT signaling pathway includes a Janus Kinase (JAK) and two Signal

Transducer and Activator of Transcription (STAT) proteins. See, e.g., Aaronson et al. Science 296.5573(2002):1653-55. The JAK family includes a number of different enzymes, including JAK1, JAK2, JAK3, and TYK2.

JAK inhibitors have been developed for treating myeloproliferative neoplasms, including ruxolitinib (INCB018424) for treating primary myelofibrosis, fedratinib (SAR302503, TG101348) for treating myelofibrosis, and XL019, SB1518 and AZD1480 for treating post-PV/ET myelofibrosis. See, e.g., Sonbol, Ther. Adv. Hematol. 4: 15-35, 2013. Patients treated with JAK inhibitors have reduced splenomegaly and/or improvement of constitutional symptoms. CYT387 (momelotinib) or N-(cyanomethyl)-4-(2-(4-morpholinophenylamino) pyrimidin-4-yl)benzamide is a JAK inhibitor that is currently in clinical trials for treating primary myelofibrosis, polycythemia vera (PV), essential thrombocythemia (ET), and post-PV/ET.

Inhibitors of JAK-STAT include a small molecule, an antibody molecule, a polypeptide, e.g., a fusion protein, or an inhibitory nucleic acid, e.g., a siRNA or shRNA.

Exemplary inhibitors of JAK-STAT include but are not limited to ruxolitinib, AG490, AZD1480, tofacitinib (tasocitinib or CP-690550), CYT387, fedratinib, baricitinib (INCB039110), lestaurtinib (CEP701), pacritinib (SB1518), XL019, gandotinib (LY2784544), BMS911543, fedratinib (SAR302503), decemotinib (V-509), INCB39110, GEN1, GEN2, GLPG0634, NS018, and N-(cyanomethyl)-4-[2-(4-morpholinoanilino)pyrimidin-4-yl]benzamide; or pharmaceutically acceptable salts thereof.

Ruxolitinib is an ATP mimetic that inhibits both JAK1 and JAK2, lowering inflammatory cytokine levels, e.g., IL-6 and TNF-alpha. See, e.g., Quintas-Cardama A. Blood 115.15(2010):3109-17. Ruxolitinib is used clinically for myelofibrosis treatment. See, e.g., Mascarenhas, J. et al. Clin. Cancer Res. 18(2012):3008-14).

In embodiments, a JAK-STAT inhibitor comprises ruxolitinib, or a pharmaceutically acceptable salt, prodrug, or solvate thereof. In one embodiment, ruxolitinib has the chemical name: (3R)-3-cyclopentyl-3-[4-(7H-pyrrolo[2,3-d]pyrimidin-4-yl)pyrazol-1-yl]propanenitrile).

In embodiments, an inhibitor of JAK-STAT includes Compound A from WO/2015/109286 (incorporated herein by reference), or a pharmaceutically acceptable salt, prodrug, or solvate thereof.

In embodiments, the JAK-STAT inhibitor is a prodrug or solvate of one or more of the JAK inhibitors listed herein.

### BTK and Inhibitors

Bruton's tyrosine kinase (BTK) is a tyrosine protein kinase that is involved in B-cell development. Inhibitors of BTK include a small molecule, an antibody molecule, a polypeptide, e.g., a fusion protein, or an inhibitory nucleic acid, e.g., a siRNA or shRNA.

In one embodiment, the kinase inhibitor is a BTK inhibitor selected from ibrutinib (PCI-32765); GDC-0834; RN-486; CGI-560; CGI-1764; HM-71224; CC-292; ONO-4059; CNX-774; and LFM-A13. In a preferred embodiment, the BTK inhibitor does not reduce or inhibit the kinase activity of interleukin-2-inducible kinase (ITK), and is selected from GDC-0834; RN-486; CGI-560; CGI-1764; HM-71224; CC-292; ONO-4059; CNX-774; and LFM-A13.

In one embodiment, the kinase inhibitor is a BTK inhibitor, e.g., ibrutinib (PCI-32765). In embodiments, a CAR-expressing cell described herein is administered to a subject in combination with a BTK inhibitor (e.g., ibrutinib). In embodiments, a CAR-expressing cell described herein is administered to a subject in combination with ibrutinib (also called PCI-32765). In one embodiment, ibrutinib has the chemical name: (1-[(3R)-3-[4-Amino-3-(4-phenoxyphenyl)-1H-pyrazolo[3,4-d]pyrimidin-1-yl]piperidin-1-yl]prop-2-en-1-one).

In embodiments, the subject has CLL, mantle cell lymphoma (MCL), or small lymphocytic lymphoma (SLL). For example, the subject has a deletion in the short arm of chromosome 17 (del(17p), e.g., in a leukemic cell). In other examples, the subject does not have a del(17p). In embodiments, the subject has relapsed CLL or SLL, e.g., the subject has previously been administered a cancer therapy (e.g., previously been administered one, two, three, or four prior cancer therapies). In embodiments, the subject has refractory CLL or SLL. In other embodiments, the subject has follicular lymphoma, e.g., relapse or refractory follicular lymphoma. In some embodiments, ibrutinib is administered at a dosage of about 300-600 mg/day (e.g., about 300-350, 350-400, 400-450, 450-500, 500-550, or 550-600 mg/day, e.g., about 420 mg/day or about 560 mg/day), e.g., orally. In embodiments, the ibrutinib is administered at a dose of about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) daily for a period of time, e.g., daily for 21 day cycle cycle, or daily for 28 day cycle. In one embodiment, 1, 2, 3, 4, 5, 6, 7, 8, 9, 10, 11, 12 or more cycles of ibrutinib are administered. In some embodiments, ibrutinib is administered in combination with rituximab. See, e.g., Burger et al. (2013) Ibrutinib In Combination With Rituximab (iR) Is Well Tolerated and Induces a High Rate Of Durable Remissions In Patients With High-Risk Chronic Lymphocytic Leukemia (CLL): New, Updated Results Of a Phase II Trial In 40 Patients, Abstract 675 presented at 55^{th} ASH Annual Meeting and Exposition, New Orleans, La. 7-10 December Without being bound by theory, it is thought that the addition of ibrutinib enhances the T cell proliferative response and may shift T cells from a T-helper-2 (Th2) to T-helper-1 (Th1) phenotype. Th1 and Th2 are phenotypes of helper T cells, with Th1 versus Th2 directing different immune response pathways. A Th1 phenotype is associated with proinflammatory responses, e.g., for killing cells, such as intracellular pathogens/viruses or cancerous cells, or perpetuating autoimmune responses. A Th2 phenotype is associated with eosinophil accumulation and anti-inflammatory responses.

In some embodiments of the methods, uses, and compositions herein, the BTK inhibitor is a BTK inhibitor described in International Application WO/2015/079417, which is herein incorporated by reference in its entirety. For instance, in some embodiments, the BTK inhibitor is a compound of formula (I) or a pharmaceutically acceptable salt thereof;

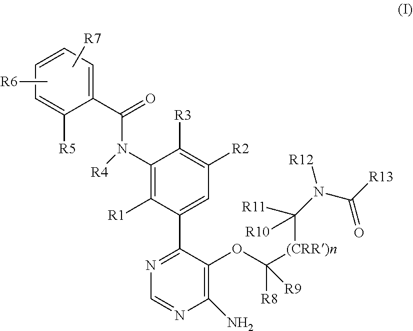

wherein,

R1 is hydrogen, C1-C6 alkyl optionally substituted by hydroxy;

R2 is hydrogen or halogen;

R3 is hydrogen or halogen;

R4 is hydrogen;

R5 is hydrogen or halogen;

or R4 and R5 are attached to each other and stand for a bond, —CH2-, -CH2-CH2-, —CH═CH—, —CH═CH—CH2-; -CH2-CH═CH—; or —CH2-CH2-CH2-;

R6 and R7 stand independently from each other for H, C1-C6 alkyl optionally substituted by hydroxyl, C3-C6 cycloalkyl optionally substituted by halogen or hydroxy, or halogen;

R8, R9, R, R′, R10 and R11 independently from each other stand for H, or C1-C6 alkyl optionally substituted by C1-C6 alkoxy; or any two of R8, R9, R, R′, R10 and R11 together with the carbon atom to which they are bound may form a 3-6 membered saturated carbocyclic ring;

R12 is hydrogen or C1-C6 alkyl optionally substituted by halogen or C1-C6 alkoxy;

or R12 and any one of R8, R9, R, R′, R10 or R11 together with the atoms to which they are bound may form a 4, 5, 6 or 7 membered azacyclic ring, which ring may optionally be substituted by halogen, cyano, hydroxyl, C1-C6 alkyl or C1-C6 alkoxy;

n is 0 or 1; and

R13 is C2-C6 alkenyl optionally substituted by C1-C6 alkyl, C1-C6 alkoxy or N,N-di-C1-C6 alkyl amino; C2-C6 alkynyl optionally substituted by C1-C6 alkyl or C1-C6 alkoxy; or C2-C6 alkylenyl oxide optionally substituted by C1-C6 alkyl.

In some embodiments, the BTK inhibitor of Formula I is chosen from: N-(3-(5-((1-Acryloylazetidin-3-yl)oxy)-6-aminopyrimidin-4-yl)-5-fluoro-2-methylphenyl)-4-cyclopropyl-2-fluorobenzamide; (E)-N-(3-(6-Amino-5-((1-(but-2-enoyl)azetidin-3-yl)oxy)pyrimidin-4-yl)-5-fluoro-2-methylphenyl)-4-cyclopropyl-2-fluorobenzamide; N-(3-(6-Amino-5-((1-propioloylazetidin-3-yl)oxy)pyrimidin-4-yl)-5-fluoro-2-methylphenyl)-4-cyclopropyl-2-fluorobenzamide; N-(3-(6-Amino-5-((1-(but-2-ynoyl)azetidin-3-yl)oxy)pyrimidin-4-yl)-5-fluoro-2-methylphenyl)-4-cyclopropyl-2-fluorobenzamide; N-(3-(5-((1-Acryloylpiperidin-4-yl)oxy)-6-aminopyrimidin-4-yl)-5-fluoro-2-methylphenyl)-4-cyclopropyl-2-fluorobenzamide; N-(3-(6-Amino-5-(2-(N-methylacrylamido)ethoxy)pyrimidin-4-yl)-5-fluoro-2-methylphenyl)-4-cyclopropyl-2-fluorobenzamide; (E)-N-(3-(6-Amino-5-(2-(N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylpropiolamido)ethoxy)pyrimidin-4-yl)-5-fluoro-2-methylphenyl)-4-cyclopropyl-2-fluorobenzamide; (E)-N-(3-(6-Amino-5-(2-(4-methoxy-N-methylbut-2-enamido)ethoxy)pyrimidin-4-yl)-5-fluoro-2-methylphenyl)-4-cyclopropyl-2-fluorobenzamide; N-(3-(6-Amino-5-(2-(N-methylbut-2-ynamido)ethoxy)pyrimidin-4-yl)-5-fluoro-2-methylphenyl)-4-cyclopropyl-2-fluorobenzamide; N-(2-((4-Amino-6-(3-(4-cyclopropyl-2-fluorobenzamido)-5-fluoro-2-methylphenyl)pyrimidin-5-yl)oxy)ethyl)-N-methyloxirane-2-carboxamide; N-(2-((4-Amino-6-(3-(6-cyclopropyl-8-fluoro-1-oxoisoquinolin-2(1H)-yl)phenyl)pyrimidin-5-yl)oxy)ethyl)-N-methylacrylamide; N-(3-(5-(2-Acrylamidoethoxy)-6-aminopyrimidin-4-yl)-5-fluoro-2-methylphenyl)-4-cyclopropyl-2-fluorobenzamide; N-(3-(6-Amino-5-(2-(N-ethylacrylamido)ethoxy)pyrimidin-4-yl)-5-fluoro-2-methylphenyl)-4-cyclopropyl-2-fluorobenzamide; N-(3-(6-Amino-5-(2-(N-(2-fluoroethyl)acrylamido)ethoxy)pyrimidin-4-yl)-5-fluoro-2-methylphenyl)-4-cyclopropyl-2-fluorobenzamide; N-(3-(5-((1-Acrylamidocyclopropyl)methoxy)-6-aminopyrimidin-4-yl)-5-fluoro-2-methylphenyl)-4-cyclopropyl-2-fluorobenzamide; (S)—N-(3-(5-(2-Acrylamidopropoxy)-6-aminopyrimidin-4-yl)-5-fluoro-2-methylphenyl)-4-cyclopropyl-2-fluorobenzamide; (S)—N-(3-(6-Amino-5-(2-(but-2-ynamido)propoxy)pyrimidin-4-yl)-5-fluoro-2-methylphenyl)-4-cyclopropyl-2-fluorobenzamide; (S)—N-(3-(6-Amino-5-(2-(N-methylacrylamido)propoxy)pyrimidin-4-yl)-5-fluoro-2-methylphenyl)-4-cyclopropyl-2-fluorobenzamide; (S)—N-(3-(6-Amino-5-(2-(N-methylbut-2-ynamido)propoxy)pyrimidin-4-yl)-5-fluoro-2-methylphenyl)-4-cyclopropyl-2-fluorobenzamide; N-(3-(6-Amino-5-(3-(N-methylacrylamido)propoxy)pyrimidin-4-yl)-5-fluoro-2-methylphenyl)-4-cyclopropyl-2-fluorobenzamide; (S)—N-(3-(5-((1-Acryloylpyrrolidin-2-yl)methoxy)-6-aminopyrimidin-4-yl)-5-fluoro-2-methylphenyl)-4-cyclopropyl-2-fluorobenzamide; (S)—N-(3-(6-Amino-5-((1-(but-2-ynoyl)pyrrolidin-2-yl)methoxy)pyrimidin-4-yl)-5-fluoro-2-methylphenyl)-4-cyclopropyl-2-fluorobenzamide; (S)-2-(3-(5-((1-Acryloylpyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(2-((4-Amino-6-(3-(6-cyclopropyl-1-oxo-3,4-dihydroisoquinolin-2(1H)-yl)-5-fluoro-2-(hydroxymethyl)phenyl)pyrimidin-5-yl)oxy)ethyl)-N-methylacrylamide; N-(3-(5-(((2S,4R)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; 2-(3-(5-(((2S,4R)-1-Acryloyl-4-methoxypyrrolidin-2-yl)methoxy)-6-aminopyrimidin-4-yl)-5-fluoro-2-(hydroxymethyl)phenyl)-6-cyclopropyl-3,4-dihydroisoquinolin-1(2H)-one; N-(3-(5-(((2S,4S)-1-Acryloyl-4-methoxypyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4S)-1-(but-2-ynoyl)-4-methoxypyrrolidin-2-yl)methoxy)pyrimidin-4-yl)-5-fluoro-2-methylphenyl)-4-cyclopropyl-2-fluorobenzamide; N-(3-(5-(((2S,4R)-1-Acryloyl-4-fluoropyrrolidin-2-yl)methoxy)-6-aminopyrimidin-4-yl)-5-fluoro-2-methylphenyl)-4-cyclopropyl-2-fluorobenzamide; N-(3-(6-Amino-5-(((2S,4R)-1-(but-2-ynoyl)-4-fluoropyrrolidin-2-yl)methoxy)pyrimidin-4-yl)-5-fluoro-2-methylphenyl)-4-cyclopropyl-2-fluorobenzamide; (S)—N-(3-(5-((1-Acryloylazetidin-2-yl)methoxy)-6-aminopyrimidin-4-yl)-5-fluoro-2-methylphenyl)-4-cyclopropyl-2-fluorobenzamide; (S)—N-(3-(6-Amino-5-((1-propioloylazetidin-2-yl)methoxy)pyrimidin-4-yl)-5-fluoro-2-methylphenyl)-4-cyclopropyl-2-fluorobenzamide; (S)-2-(3-(5-((1-Acryloylazetidin-2-yl)methoxy)-6-aminopyrimidin-4-yl)-5-fluoro-2-(hydroxymethyl)phenyl)-6-cyclopropyl-3,4-dihydroisoquinolin-1(2H)-one; (R)—N-(3-(5-((1-Acryloylazetidin-2-yl)methoxy)-6-aminopyrimidin-4-yl)-5-fluoro-2-methylphenyl)-4-cyclopropyl-2-fluorobenzamide; (R)—N-(3-(5-((1-Acryloylpiperidin-3-yl)methoxy)-6-aminopyrimidin-4-yl)-5-fluoro-2-methylphenyl)-4-cyclopropyl-2-fluorobenzamide; N-(3-(5-(((2R,3S)-1-Acryloyl-3-methoxypyrrolidin-2-yl)methoxy)-6-aminopyrimidin-4-yl)-5-fluoro-2-methylphenyl)-4-cyclopropyl-2-fluorobenzamide; N-(3-(5-(((2S,4R)-1-Acryloyl-4-cyanopyrrolidin-2-yl)methoxy)-6-aminopyrimidin-4-yl)-5-fluoro-2-methylphenyl)-4-cyclopropyl-2-fluorobenzamide; or N-(3-(5-(((2S,4S)-1-Acryloyl-4-cyanopyrrolidin-2-yl)methoxy)-6-aminopyrimidin-4-yl)-5-fluoro-2-methylphenyl)-4-cyclopropyl-2-fluorobenzamide.

Unless otherwise provided, the chemical terms used above in describing the BTK inhibitor of Formula I are used according to their meanings as set out in International Application WO/2015/079417, which is herein incorporated by reference in its entirety.

Additional examples of BTK inhibitors are described herein, e.g., in the Further Combination Therapies section herein.

### Further Combination Therapies

In further aspects, a CAR-expressing cell described herein may be used in a treatment regimen in combination with surgery, cytokines, radiation, or chemotherapy such as cytoxan, fludarabine, histone deacetylase inhibitors, demethylating agents, or peptide vaccine, such as that described in Izumoto et al. 2008 J Neurosurg 108:963-971.

In certain instances, compounds of the present invention are combined with other therapeutic agents, such as other anti-cancer agents, anti-allergic agents, anti-nausea agents (or anti-emetics), pain relievers, cytoprotective agents, and combinations thereof.

In one embodiment, a CAR-expressing cell and/or the STAT/JAK inhibitor or BTK inhibitor described herein can be used further in combination with a chemotherapeutic agent. Exemplary chemotherapeutic agents include an anthracycline (e.g., doxorubicin (e.g., liposomal doxorubicin)). a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for the combinations disclosed herein include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In embodiments, the combinations disclosed herein include fludarabine, cyclophosphamide, and/or rituximab. In embodiments, the combinations disclosed herein include fludarabine, cyclophosphamide, and rituximab (FCR). In embodiments, the subject has CLL. For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the fludarabine is administered at a dosage of about 10-50 mg/m² (e.g., about 10-15, 15-20, 20-25, 25-30, 30-35, 35-40, 40-45, or 45-50 mg/m²), e.g., intravenously. In embodiments, the cyclophosphamide is administered at a dosage of about 200-300 mg/m² (e.g., about 200-225, 225-250, 250-275, or 275-300 mg/m²), e.g., intravenously. In embodiments, the rituximab is administered at a dosage of about 400-600 mg/m2 (e.g., 400-450, 450-500, 500-550, or 550-600 mg/m²), e.g., intravenously.

In embodiments, the combinations disclosed herein include bendamustine and rituximab. In embodiments, the subject has CLL. For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the bendamustine is administered at a dosage of about 70-110 mg/m2 (e.g., 70-80, 80-90, 90-100, or 100-110 mg/m2), e.g., intravenously. In embodiments, the rituximab is administered at a dosage of about 400-600 mg/m2 (e.g., 400-450, 450-500, 500-550, or 550-600 mg/m²), e.g., intravenously.

In embodiments, the combinations disclosed herein include rituximab, cyclophosphamide, doxorubicine, vincristine, and/or a corticosteroid (e.g., prednisone). In embodiments, a CAR-expressing cell described herein is administered to a subject in combination with rituximab, cyclophosphamide, doxorubicine, vincristine, and prednisone (R-CHOP). In embodiments, the subject has diffuse large B-cell lymphoma (DLBCL). In embodiments, the subject has nonbulky limited-stage DLBCL (e.g., comprises a tumor having a size/diameter of less than 7 cm). In embodiments, the subject is treated with radiation in combination with the R-CHOP. For example, the subject is administered R-CHOP (e.g., 1-6 cycles, e.g., 1, 2, 3, 4, 5, or 6 cycles of R-CHOP), followed by radiation. In some cases, the subject is administered R-CHOP (e.g., 1-6 cycles, e.g., 1, 2, 3, 4, 5, or 6 cycles of R-CHOP) following radiation.

In embodiments, the combinations disclosed herein include etoposide, prednisone, vincristine, cyclophosphamide, doxorubicin, and/or rituximab. In embodiments, a CAR-expressing cell described herein is administered to a subject in combination with etoposide, prednisone, vincristine, cyclophosphamide, doxorubicin, and rituximab (EPOCH-R). In embodiments, a CAR-expressing cell described herein is administered to a subject in combination with dose-adjusted EPOCH-R (DA-EPOCH-R). In embodiments, the subject has a B cell lymphoma, e.g., a Myc-rearranged aggressive B cell lymphoma.

In embodiments, the combinations disclosed herein include rituximab and/or lenalidomide. Lenalidomide ((RS)-3-(4-Amino-1-oxo 1,3-dihydro-2H-isoindol-2-yl)piperidine-2,6-dione) is an immunomodulator. In embodiments, a CAR-expressing cell described herein is administered to a subject in combination with rituximab and lenalidomide. In embodiments, the subject has follicular lymphoma (FL) or mantle cell lymphoma (MCL). In embodiments, the subject has FL and has not previously been treated with a cancer therapy. In embodiments, lenalidomide is administered at a dosage of about 10-20 mg (e.g., 10-15 or 15-20 mg), e.g., daily. In embodiments, rituximab is administered at a dosage of about 350-550 mg/m² (e.g., 350-375, 375-400, 400-425, 425-450, 450-475, or 475-500 mg/m²), e.g., intravenously.

Exemplary mTOR inhibitors include, e.g., temsirolimus; ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0⁴′⁹] hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RAD001); rapamycin (AY22989, Sirolimus®); simapimod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-(SEQ ID NO: 706), inner salt (SF1126, CAS 936487-67-1), and XL765.

Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics).

Exemplary anthracyclines include, e.g., doxorubicin (Adriamycin® and Rubex®); bleomycin (Lenoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin.

Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Exemplary proteosome inhibitors include bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); and O-Methyl-N-[(2-methyl-5-thiazolyl)carbonyl]-L-seryl-O-methyl-N—R1S)-2-[(2R)-2-methyl-2-oxiranyl]-2-oxo-1-(phenylmethyl)ethyl]-L-serinamide (ONX-0912).

In embodiments, a CAR-expressing cell described herein is administered to a subject in combination with brentuximab. Brentuximab is an antibody-drug conjugate of anti-CD30 antibody and monomethyl auristatin E. In embodiments, the subject has Hodgkin's lymphoma (HL), e.g., relapsed or refractory HL. In embodiments, the subject comprises CD30+HL. In embodiments, the subject has undergone an autologous stem cell transplant (ASCT). In embodiments, the subject has not undergone an ASCT. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In embodiments, a CAR-expressing cell described herein is administered to a subject in combination with brentuximab and dacarbazine or in combination with brentuximab and bendamustine. Dacarbazine is an alkylating agent with a chemical name of 5-(3,3-Dimethyl-1-triazenyl)imidazole-4-carboxamide. Bendamustine is an alkylating agent with a chemical name of 4-[5-[Bis(2-chloroethyl)amino]-1-methylbenzimidazol-2-yl]butanoic acid. In embodiments, the subject has Hodgkin's lymphoma (HL). In embodiments, the subject has not previously been treated with a cancer therapy. In embodiments, the subject is at least 60 years of age, e.g., 60, 65, 70, 75, 80, 85, or older. In embodiments, dacarbazine is administered at a dosage of about 300-450 mg/m² (e.g., about 300-325, 325-350, 350-375, 375-400, 400-425, or 425-450 mg/m²), e.g., intravenously. In embodiments, bendamustine is administered at a dosage of about 75-125 mg/m2 (e.g., 75-100 or 100-125 mg/m², e.g., about 90 mg/m²), e.g., intravenously. In embodiments, brentuximab is administered at a dosage of about 1-3 mg/kg (e.g., about 1-1.5, 1.5-2, 2-2.5, or 2.5-3 mg/kg), e.g., intravenously, e.g., every 3 weeks.

In some embodiments, a CAR-expressing cell described herein is administered to a subject in combination with a CD20 inhibitor, e.g., an anti-CD20 antibody (e.g., an anti-CD20 mono- or bispecific antibody) or a fragment thereof. Exemplary anti-CD20 antibodies include but are not limited to rituximab, ofatumumab, ocrelizumab, veltuzumab, obinutuzumab, TRU-015 (Trubion Pharmaceuticals), ocaratuzumab, and Pro131921 (Genentech). See, e.g., Lim et al. Haematologica. 95.1(2010):135-43.

In some embodiments, the anti-CD20 antibody comprises rituximab. Rituximab is a chimeric mouse/human monoclonal antibody IgG1 kappa that binds to CD20 and causes cytolysis of a CD20 expressing cell, e.g., as described in www.accessdata.fda.gov/drugsatfda_docs/label/2010/103705s53111bl.pdf. In embodiments, a CAR-expressing cell described herein is administered to a subject in combination with rituximab. In embodiments, the subject has CLL or SLL.

In some embodiments, rituximab is administered intravenously, e.g., as an intravenous infusion. For example, each infusion provides about 500-2000 mg (e.g., about 500-550, 550-600, 600-650, 650-700, 700-750, 750-800, 800-850, 850-900, 900-950, 950-1000, 1000-1100, 1100-1200, 1200-1300, 1300-1400, 1400-1500, 1500-1600, 1600-1700, 1700-1800, 1800-1900, or 1900-2000 mg) of rituximab. In some embodiments, rituximab is administered at a dose of 150 mg/m² to 750 mg/m², e.g., about 150-175 mg/m², 175-200 mg/m², 200-225 mg/m², 225-250 mg/m², 250-300 mg/m², 300-325 mg/m², 325-350 mg/m², 350-375 mg/m², 375-400 mg/m², 400-425 mg/m², 425-450 mg/m², 450-475 mg/m², 475-500 mg/m², 500-525 mg/m², 525-550 mg/m², 550-575 mg/m², 575-600 mg/m², 600-625 mg/m², 625-650 mg/m², 650-675 mg/m², or 675-700 mg/m², where m² indicates the body surface area of the subject. In some embodiments, rituximab is administered at a dosing interval of at least 4 days, e.g., 4, 7, 14, 21, 28, 35 days, or more. For example, rituximab is administered at a dosing interval of at least 0.5 weeks, e.g., 0.5, 1, 2, 3, 4, 5, 6, 7, 8 weeks, or more. In some embodiments, rituximab is administered at a dose and dosing interval described herein for a period of time, e.g., at least 2 weeks, e.g., at least 2, 3, 4, 5, 6, 7, 8, 9, 10, 11, 12, 13, 14, 15, 16, 17, 18, 19, 20 weeks, or greater. For example, rituximab is administered at a dose and dosing interval described herein fora total of at least 4 doses per treatment cycle (e.g., at least 4, 5, 6, 7, 8, 9, 10, 11, 12, 13, 14, 15, 16, or more doses per treatment cycle).

In some embodiments, the anti-CD20 antibody comprises ofatumumab. Ofatumumab is an anti-CD20 IgG1κ human monoclonal antibody with a molecular weight of approximately 149 kDa. For example, ofatumumab is generated using transgenic mouse and hybridoma technology and is expressed and purified from a recombinant murine cell line (NSO). See, e.g., www.accessdata.fda.gov/drugsatfda_docs/label/2009/1253261bl.pdf; and Clinical Trial Identifier number NCT01363128, NCT01515176, NCT01626352, and NCT01397591. In embodiments, a CAR-expressing cell described herein is administered to a subject in combination with ofatumumab. In embodiments, the subject has CLL or SLL.

In some embodiments, ofatumumab is administered as an intravenous infusion. For example, each infusion provides about 150-3000 mg (e.g., about 150-200, 200-250, 250-300, 300-350, 350-400, 400-450, 450-500, 500-550, 550-600, 600-650, 650-700, 700-750, 750-800, 800-850, 850-900, 900-950, 950-1000, 1000-1200, 1200-1400, 1400-1600, 1600-1800, 1800-2000, 2000-2200, 2200-2400, 2400-2600, 2600-2800, or 2800-3000 mg) of ofatumumab. In embodiments, ofatumumab is administered at a starting dosage of about 300 mg, followed by 2000 mg, e.g., for about 11 doses, e.g., for 24 weeks. In some embodiments, ofatumumab is administered at a dosing interval of at least 4 days, e.g., 4, 7, 14, 21, 28, 35 days, or more. For example, ofatumumab is administered at a dosing interval of at least 1 week, e.g., 1, 2, 3, 4, 5, 6, 7, 8, 9, 10, 11, 12, 24, 26, 28, 20, 22, 24, 26, 28, 30 weeks, or more. In some embodiments, ofatumumab is administered at a dose and dosing interval described herein for a period of time, e.g., at least 1 week, e.g., 1, 2, 3, 4, 5, 6, 7, 8, 9, 10, 11, 12, 13, 14, 15, 16, 17, 18, 19, 20, 22, 24, 26, 28, 30, 40, 50, 60 weeks or greater, or 1, 2, 3, 4, 5, 6, 7, 8, 9, 10, 11, 12 months or greater, or 1, 2, 3, 4, 5 years or greater. For example, ofatumumab is administered at a dose and dosing interval described herein for a total of at least 2 doses per treatment cycle (e.g., at least 2, 3, 4, 5, 6, 7, 8, 9, 10, 11, 12, 13, 14, 15, 16, 18, 20, or more doses per treatment cycle).

In some cases, the anti-CD20 antibody comprises ocrelizumab. Ocrelizumab is a humanized anti-CD20 monoclonal antibody, e.g., as described in Clinical Trials Identifier Nos. NCT00077870, NCT01412333, NCT00779220, NCT00673920, NCT01194570, and Kappos et al. Lancet. 19.378(2011):1779-87.

In some cases, the anti-CD20 antibody comprises veltuzumab. Veltuzumab is a humanized monoclonal antibody against CD20. See, e.g., Clinical Trial Identifier No. NCT00547066, NCT00546793, NCT01101581, and Goldenberg et al. Leuk Lymphoma. 51(5)(2010):747-55.

In some cases, the anti-CD20 antibody comprises GA101. GA101 (also called obinutuzumab or R05072759) is a humanized and glyco-engineered anti-CD20 monoclonal antibody. See, e.g., Robak. Curr. Opin. Investig. Drugs. 10.6(2009):588-96; Clinical Trial Identifier Numbers: NCT01995669, NCT01889797, NCT02229422, and NCT01414205; and www.accessdata.fda.gov/drugsatfda_docs/label/2013/125486s0001bl.pdf.

In some cases, the anti-CD20 antibody comprises AME-133v. AME-133v (also called LY2469298 or ocaratuzumab) is a humanized IgG1 monoclonal antibody against CD20 with increased affinity for the FcγRIIIa receptor and an enhanced antibody dependent cellular cytotoxicity (ADCC) activity compared with rituximab. See, e.g., Robak et al. BioDrugs 25.1(2011):13-25; and Forero-Torres et al. Clin Cancer Res. 18.5(2012):1395-403.

In some cases, the anti-CD20 antibody comprises PRO131921. PRO131921 is a humanized anti-CD20 monoclonal antibody engineered to have better binding to FcγRIIIa and enhanced ADCC compared with rituximab. See, e.g., Robak et al. BioDrugs 25.1(2011):13-25; and Casulo et al. Clin Immunol. 154.1(2014):37-46; and Clinical Trial Identifier No. NCT00452127.

In some cases, the anti-CD20 antibody comprises TRU-015. TRU-015 is an anti-CD20 fusion protein derived from domains of an antibody against CD20. TRU-015 is smaller than monoclonal antibodies, but retains Fc-mediated effector functions. See, e.g., Robak et al. BioDrugs 25.1(2011):13-25. TRU-015 contains an anti-CD20 single-chain variable fragment (scFv) linked to human IgG1 hinge, CH2, and CH3 domains but lacks CH1 and CL domains.

In some embodiments, an anti-CD20 antibody described herein is conjugated or otherwise bound to a therapeutic agent, e.g., a chemotherapeutic agent (e.g., cytoxan, fludarabine, histone deacetylase inhibitor, demethylating agent, peptide vaccine, anti-tumor antibiotic, tyrosine kinase inhibitor, alkylating agent, anti-microtubule or anti-mitotic agent), anti-allergic agent, anti-nausea agent (or anti-emetic), pain reliever, or cytoprotective agent described herein.

In embodiments, the combinations disclosed herein include a B-cell lymphoma 2 (BCL-2) inhibitor (e.g., venetoclax, also called ABT-199 or GDC-0199) and/or rituximab. In embodiments, a CAR-expressing cell described herein is administered to a subject in combination with venetoclax and rituximab. Venetoclax is a small molecule that inhibits the anti-apoptotic protein, BCL-2. In one embodiment, venetoclax has the chemical name: (4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide).

In embodiments, the subject has CLL. In embodiments, the subject has relapsed CLL, e.g., the subject has previously been administered a cancer therapy. In embodiments, venetoclax is administered at a dosage of about 15-600 mg (e.g., 15-20, 20-50, 50-75, 75-100, 100-200, 200-300, 300-400, 400-500, or 500-600 mg), e.g., daily. In embodiments, rituximab is administered at a dosage of about 350-550 mg/m2 (e.g., 350-375, 375-400, 400-425, 425-450, 450-475, or 475-500 mg/m2), e.g., intravenously, e.g., monthly.

In some embodiments, the combinations disclosed herein include an oncolytic virus. In embodiments, oncolytic viruses are capable of selectively replicating in and triggering the death of or slowing the growth of a cancer cell. In some cases, oncolytic viruses have no effect or a minimal effect on non-cancer cells. An oncolytic virus includes but is not limited to an oncolytic adenovirus, oncolytic Herpes Simplex Viruses, oncolytic retrovirus, oncolytic parvovirus, oncolytic vaccinia virus, oncolytic Sinbis virus, oncolytic influenza virus, or oncolytic RNA virus (e.g., oncolytic reovirus, oncolytic Newcastle Disease Virus (NDV), oncolytic measles virus, or oncolytic vesicular stomatitis virus (VSV)).

In some embodiments, the oncolytic virus is a virus, e.g., recombinant oncolytic virus, described in US2010/0178684 A1, which is incorporated herein by reference in its entirety. In some embodiments, a recombinant oncolytic virus comprises a nucleic acid sequence (e.g., heterologous nucleic acid sequence) encoding an inhibitor of an immune or inflammatory response, e.g., as described in US2010/0178684 A1, incorporated herein by reference in its entirety. In embodiments, the recombinant oncolytic virus, e.g., oncolytic NDV, comprises a pro-apoptotic protein (e.g., apoptin), a cytokine (e.g., GM-CSF, interferon-gamma, interleukin-2 (IL-2), tumor necrosis factor-alpha), an immunoglobulin (e.g., an antibody against ED-B firbonectin), tumor associated antigen, a bispecific adapter protein (e.g., bispecific antibody or antibody fragment directed against NDV HN protein and a T cell co-stimulatory receptor, such as CD3 or CD28; or fusion protein between human IL-2 and single chain antibody directed against NDV HN protein). See, e.g., Zamarin et al. Future Microbiol. 7.3(2012):347-67, incorporated herein by reference in its entirety. In some embodiments, the oncolytic virus is a chimeric oncolytic NDV described in U.S. Pat. No. 8,591,881 B2, US 2012/0122185 A1, or US 2014/0271677 A1, each of which is incorporated herein by reference in their entireties.

In some embodiments, the oncolytic virus comprises a conditionally replicative adenovirus (CRAd), which is designed to replicate exclusively in cancer cells. See, e.g., Alemany et al. Nature Biotechnol. 18(2000):723-27. In some embodiments, an oncolytic adenovirus comprises one described in Table 1 on page 725 of Alemany et al., incorporated herein by reference in its entirety.

Exemplary oncolytic viruses include but are not limited to the following:

Group B Oncolytic Adenovirus (ColoAd1) (PsiOxus Therapeutics Ltd.) (see, e.g., Clinical Trial Identifier: NCT02053220);

ONCOS-102 (previously called CGTG-102), which is an adenovirus comprising granulocyte-macrophage colony stimulating factor (GM-CSF) (Oncos Therapeutics) (see, e.g., Clinical Trial Identifier: NCT01598129);

VCN-01, which is a genetically modified oncolytic human adenovirus encoding human PH20 hyaluronidase (VCN Biosciences, S.L.) (see, e.g., Clinical Trial Identifiers: NCT02045602 and NCT02045589);

Conditionally Replicative Adenovirus ICOVIR-5, which is a virus derived from wild-type human adenovirus serotype 5 (Had5) that has been modified to selectively replicate in cancer cells with a deregulated retinoblastoma/E2F pathway (Institut Català d′Oncologia) (see, e.g., Clinical Trial Identifier: NCT01864759);

Celyvir, which comprises bone marrow-derived autologous mesenchymal stem cells (MSCs) infected with ICOVIR5, an oncolytic adenovirus (Hospital Infantil Universitario Niño Jesús, Madrid, Spain/Ramon Alemany) (see, e.g., Clinical Trial Identifier: NCT01844661);

CG0070, which is a conditionally replicating oncolytic serotype 5 adenovirus (Ad5) in which human E2F-1 promoter drives expression of the essential Ela viral genes, thereby restricting viral replication and cytotoxicity to Rb pathway-defective tumor cells (Cold Genesys, Inc.) (see, e.g., Clinical Trial Identifier: NCT02143804); or

DNX-2401 (formerly named Delta-24-RGD), which is an adenovirus that has been engineered to replicate selectively in retinoblastoma (Rb)-pathway deficient cells and to infect cells that express certain RGD-binding integrins more efficiently (Clinica Universidad de Navarra, Universidad de Navarra/DNAtrix, Inc.) (see, e.g., Clinical Trial Identifier: NCT01956734).

In some embodiments, an oncolytic virus described herein is administering by injection, e.g., subcutaneous, intra-arterial, intravenous, intramuscular, intrathecal, or intraperitoneal injection. In embodiments, an oncolytic virus described herein is administered intratumorally, transdermally, transmucosally, orally, intranasally, or via pulmonary administration.

In an embodiment, cells expressing a CAR described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse.

In one embodiment, the combinations disclosed herein include a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In one embodiment, the GITR binding molecules and/or molecules modulating GITR functions (e.g., GITR agonist and/or Treg depleting GITR antibodies) are administered prior to administration of the CAR-expressing cell. For example, in one embodiment, the GITR agonist can be administered prior to apheresis of the cells. In embodiments, cyclophosphamide is administered to the subject prior to administration (e.g., infusion or re-infusion) of the CAR-expressing cell or prior to aphersis of the cells. In embodiments, cyclophosphamide and an anti-GITR antibody are administered to the subject prior to administration (e.g., infusion or re-infusion) of the CAR-expressing cell or prior to apheresis of the cells. In one embodiment, the subject has cancer (e.g., a solid cancer or a hematological cancer such as ALL or CLL). In an embodiment, the subject has CLL. In embodiments, the subject has ALL. In embodiments, the subject has a solid cancer, e.g., a solid cancer described herein. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.:WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, a CAR expressing cell described herein is administered to a subject in combination with a GITR agonist, e.g., a GITR agonist described herein. In one embodiment, the GITR agonist is administered prior to the CAR-expressing cell. For example, in one embodiment, the GITR agonist can be administered prior to apheresis of the cells.

In one embodiment, the combinations disclosed herein include an mTOR inhibitor, e.g., an mTOR inhibitor described herein, e.g., a rapalog such as everolimus. In one embodiment, the mTOR inhibitor is administered prior to the CAR-expressing cell. For example, in one embodiment, the mTOR inhibitor can be administered prior to apheresis of the cells.

In one embodiment the combinations disclosed herein include a protein tyrosine phosphatase inhibitor, e.g., a protein tyrosine phosphatase inhibitor described herein. In one embodiment, the protein tyrosine phosphatase inhibitor is an SHP-1 inhibitor, e.g., an SHP-1 inhibitor described herein, such as, e.g., sodium stibogluconate. In one embodiment, the protein tyrosine phosphatase inhibitor is an SHP-2 inhibitor.

In one embodiment, the combinations disclosed herein include a kinase inhibitor other than JAK/STAT inhibitor or BTK inhibitor. In one embodiment, the kinase inhibitor is a CDK4 inhibitor, e.g., a CDK4 inhibitor described herein, e.g., a CD4/6 inhibitor, such as, e.g., 6-Acetyl-8-cyclopentyl-5-methyl-2-(5-piperazin-1-yl-pyridin-2-ylamino)-8H-pyrido[2,3-d]pyrimidin-7-one, hydrochloride (also referred to as palbociclib or PD0332991). In one embodiment, the kinase inhibitor is an mTOR inhibitor, e.g., an mTOR inhibitor described herein, such as, e.g., rapamycin, a rapamycin analog, OSI-027. The mTOR inhibitor can be, e.g., an mTORC1 inhibitor and/or an mTORC2 inhibitor, e.g., an mTORC1 inhibitor and/or mTORC2 inhibitor described herein. In one embodiment, the kinase inhibitor is a MNK inhibitor, e.g., a MNK inhibitor described herein, such as, e.g., 4-amino-5-(4-fluoroanilino)-pyrazolo [3,4-d] pyrimidine. The MNK inhibitor can be, e.g., a MNK1a, MNK1b, MNK2a and/or MNK2b inhibitor. In one embodiment, the kinase inhibitor is a DGK inhibitor, e.g., a DGK inhibitor described herein, such as, e.g., DGKinh1 (D5919) or DGKinh2 (D5794). In one embodiment, the kinase inhibitor is a CDK4 inhibitor selected from aloisine A; flavopiridol or HMR-1275, 2-(2-chlorophenyl)-5,7-dihydroxy-8-[(3S,4R)-3-hydroxy-1-methyl-4-piperidinyl]-4-chromenone; crizotinib (PF-02341066; 2-(2-Chlorophenyl)-5,7-dihydroxy-8-[(2R,3S)-2-(hydroxymethyl)-1-methyl-3-pyrrolidinyl]-4H-1-benzopyran-4-one, hydrochloride (P276-00); 1-methyl-5-[[2-[5-(trifluoromethyl)-1H-imidazol-2-yl]-4-pyridinyl]oxy]-N-[4-(trifluoromethyl)phenyl]-1H-benzimidazol-2-amine (RAF265); indisulam (E7070); roscovitine (CYC202); palbociclib (PD0332991); dinaciclib (SCH727965); N-[5-[[(5-tert-butyloxazol-2-yl)methyl]thio]thiazol-2-yl]piperidine-4-carboxamide (BMS 387032); 44-[9-chloro-7-(2,6-difluorophenyl)-5H-pyrimido[5,4-d][2]benzazepin-2-yl]amino]-benzoic acid (MLN8054); 5-[3-(4,6-difluoro-1H-benzimidazol-2-yl)-1H-indazol-5-yl]-N-ethyl-4-methyl-3-pyridinemethanamine (AG-024322); 4-(2,6-dichlorobenzoylamino)-1H-pyrazole-3-carboxylic acid N-(piperidin-4-yl)amide (AT7519); 4-[2-methyl-1-(1-methylethyl)-1H-imidazol-5-yl]-N-[4-(methylsulfonyl)phenyl]-2-pyrimidinamine (AZD5438); and XL281 (BMS908662).

In one embodiment, the kinase inhibitor is a CDK4 inhibitor, e.g., palbociclib (PD0332991), and the palbociclib is administered at a dose of about 50 mg, 60 mg, 70 mg, 75 mg, 80 mg, 90 mg, 100 mg, 105 mg, 110 mg, 115 mg, 120 mg, 125 mg, 130 mg, 135 mg (e.g., 75 mg, 100 mg or 125 mg) daily for a period of time, e.g., daily for 14-21 days of a 28 day cycle, or daily for 7-12 days of a 21 day cycle. In one embodiment, 1, 2, 3, 4, 5, 6, 7, 8, 9, 10, 11, 12 or more cycles of palbociclib are administered.

In embodiments, a CAR-expressing cell described herein is administered to a subject in combination with a cyclin-dependent kinase (CDK) 4 or 6 inhibitor, e.g., a CDK4 inhibitor or a CDK6 inhibitor described herein. In embodiments, a CAR-expressing cell described herein is administered to a subject in combination with a CDK4/6 inhibitor (e.g., an inhibitor that targets both CDK4 and CDK6), e.g., a CDK4/6 inhibitor described herein. In an embodiment, the subject has MCL. MCL is an aggressive cancer that is poorly responsive to currently available therapies, i.e., essentially incurable. In many cases of MCL, cyclin D1 (a regulator of CDK4/6) is expressed (e.g., due to chromosomal translocation involving immunoglobulin and Cyclin D1 genes) in MCL cells. Thus, without being bound by theory, it is thought that MCL cells are highly sensitive to CDK4/6 inhibition with high specificity (i.e., minimal effect on normal immune cells). CDK4/6 inhibitors alone have had some efficacy in treating MCL, but have only achieved partial remission with a high relapse rate. An exemplary CDK4/6 inhibitor is LEE011 (also called ribociclib), the structure of which is shown below.

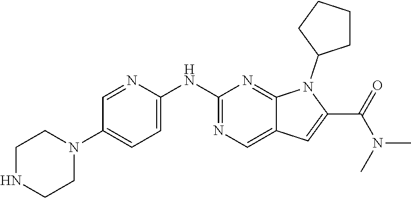 [SMILES: CN(C)C(=O)c1cc2cnc(Nc3ccc(N4CCNCC4)cn3)nc2n1C1CCCC1]

Without being bound by theory, it is believed that administration of a CAR-expressing cell described herein with a CDK4/6 inhibitor (e.g., LEE011 or other CDK4/6 inhibitors described herein) can achieve higher responsiveness, e.g., with higher remission rates and/or lower relapse rates, e.g., compared to a CDK4/6 inhibitor alone.

In one embodiment, the kinase inhibitor is an mTOR inhibitor selected from temsirolimus; ridaforolimus (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4,9}] hexatriaconta-16,24,26,28-tetraen-12-yl]propyl]-2-methoxycyclohexyl dimethylphosphinate, also known as AP23573 and MK8669; everolimus (RAD001); rapamycin (AY22989); simapimod; (5-{2,4-bis[(3S)-3-methylmorpholin-4-yl]pyrido[2,3-d]pyrimidin-7-yl}-2-methoxyphenyl)methanol (AZD8055); 2-mmino-8-[trans-4-(2-hydroxyethoxy)cyclohexyl]-6-(6-methoxy-3-pyridinyl)-4-methyl-pyrido[2,3-d]pyrimidin-7(8H)-one (PF04691502); and N²-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine- (SEQ ID NO: 706), inner salt (SF1126); and XL765.

In one embodiment, the kinase inhibitor is an mTOR inhibitor, e.g., rapamycin, and the rapamycin is administered at a dose of about 3 mg, 4 mg, 5 mg, 6 mg, 7 mg, 8 mg, 9 mg, 10 mg (e.g., 6 mg) daily for a period of time, e.g., daily for 21 day cycle cycle, or daily for 28 day cycle. In one embodiment, 1, 2, 3, 4, 5, 6, 7, 8, 9, 10, 11, 12 or more cycles of rapamycin are administered. In one embodiment, the kinase inhibitor is an mTOR inhibitor, e.g., everolimus and the everolimus is administered at a dose of about 2 mg, 2.5 mg, 3 mg, 4 mg, 5 mg, 6 mg, 7 mg, 8 mg, 9 mg, 10 mg, 11 mg, 12 mg, 13 mg, 14 mg, 15 mg (e.g., 10 mg) daily fora period of time, e.g., daily for 28 day cycle. In one embodiment, 1, 2, 3, 4, 5, 6, 7, 8, 9, 10, 11, 12 or more cycles of everolimus are administered.

In one embodiment, the kinase inhibitor is an MNK inhibitor selected from CGP052088; 4-amino-3-(p-fluorophenylamino)-pyrazolo [3,4-d] pyrimidine (CGP57380); cercosporamide; ETC-1780445-2; and 4-amino-5-(4-fluoroanilino)-pyrazolo [3,4-d] pyrimidine.

In embodiments, a CAR-expressing cell described herein is administered to a subject in combination with a phosphoinositide 3-kinase (PI3K) inhibitor (e.g., a PI3K inhibitor described herein, e.g., idelalisib or duvelisib) and/or rituximab. In embodiments, a CAR-expressing cell described herein is administered to a subject in combination with idelalisib and rituximab. In embodiments, a CAR-expressing cell described herein is administered to a subject in combination with duvelisib and rituximab. Idelalisib (also called GS-1101 or CAL-101; Gilead) is a small molecule that blocks the delta isoform of PI3K. In an embodiment, idelalisib has the chemical name: (5-Fluoro-3-phenyl-2-R1S)-1-(7H-purin-6-ylamino)propyl]-4(3H)-quinazolinone).

Duvelisib (also called IPI-145; Infinity Pharmaceuticals) is a small molecule that blocks PI3K-δ,γ. In an embodiment, duvelisib has the chemical name: (8-Chloro-2-phenyl-3-[(1S)-1-(9H-purin-6-ylamino)ethyl]-1(2H)-isoquinolinone).

In embodiments, the subject has CLL. In embodiments, the subject has relapsed CLL, e.g., the subject has previously been administered a cancer therapy (e.g., previously been administered an anti-CD20 antibody or previously been administered ibrutinib). For example, the subject has a deletion in the short arm of chromosome 17 (del(17p), e.g., in a leukemic cell). In other examples, the subject does not have a del(17p). In embodiments, the subject comprises a leukemic cell comprising a mutation in the immunoglobulin heavy-chain variable-region (IgV_{H}) gene. In other embodiments, the subject does not comprise a leukemic cell comprising a mutation in the immunoglobulin heavy-chain variable-region (IgV_{H}) gene. In embodiments, the subject has a deletion in the long arm of chromosome 11 (del(11q)). In other embodiments, the subject does not have a del(11q). In embodiments, idelalisib is administered at a dosage of about 100-400 mg (e.g., 100-125, 125-150, 150-175, 175-200, 200-225, 225-250, 250-275, 275-300, 325-350, 350-375, or 375-400 mg), e.g., BID. In embodiments, duvelisib is administered at a dosage of about 15-100 mg (e.g., about 15-25, 25-50, 50-75, or 75-100 mg), e.g., twice a day. In embodiments, rituximab is administered at a dosage of about 350-550 mg/m² (e.g., 350-375, 375-400, 400-425, 425-450, 450-475, or 475-500 mg/m²), e.g., intravenously.

In one embodiment, the kinase inhibitor is a dual phosphatidylinositol 3-kinase (PI3K) and mTOR inhibitor selected from 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF-04691502); N-[4-[[4-(Dimethylamino)-1-piperidinyl]carbonyl]phenyl]-N′-[4-(4,6-di-4-morpholinyl-1,3,5-triazin-2-yl)phenyl]urea (PF-05212384, PKI-587); 2-Methyl-2-{4-[3-methyl-2-oxo-8-(quinolin-3-yl)-2,3-dihydro-1H-imidazo[4,5-c]quinolin-1-yl]phenyl}propanenitrile (BEZ-235); apitolisib (GDC-0980, RG7422); 2,4-Difluoro-N-{2-(methyloxy)-5-[4-(4-pyridazinyl)-6-quinolinyl]-3-pyridinyl}benzenesulfonamide (GSK2126458); 8-(6-methoxypyridin-3-yl)-3-methyl-1-(4-(piperazin-1-yl)-3-(trifluoromethyl)phenyl)-1H-imidazo[4,5-c]quinolin-2(3H)-one Maleic acid (NVP-BGT226); 3-[4-(4-Morpholinylpyrido[3′,2′:4,5]furo[3,2-d]pyrimidin-2-yl]phenol (PI-103); 5-(9-isopropyl-8-methyl-2-morpholino-9H-purin-6-yl)pyrimidin-2-amine (VS-5584, SB2343); and N42-[(3,5-Dimethoxyphenyl)amino]quinoxalin-3-yl]-4-[(4-methyl-3-methoxyphenyl)carbonyl]aminophenylsulfonamide (XL765).

In embodiments, a CAR-expressing cell described herein is administered to a subject in combination with an anaplastic lymphoma kinase (ALK) inhibitor. Exemplary ALK kinases include but are not limited to crizotinib (Pfizer), ceritinib (Novartis), alectinib (Chugai), brigatinib (also called AP26113; Ariad), entrectinib (Ignyta), PF-06463922 (Pfizer), TSR-011 (Tesaro) (see, e.g., Clinical Trial Identifier No. NCT02048488), CEP-37440 (Teva), and X-396 (Xcovery). In some embodiments, the subject has a solid cancer, e.g., a solid cancer described herein, e.g., lung cancer.

The chemical name of crizotinib is 3-[(1R)-1-(2,6-dichloro-3-fluorophenyl)ethoxy]-5-(1-piperidin-4-ylpyrazol-4-yl)pyridin-2-amine. The chemical name of ceritinib is 5-Chloro-N²-[2-isopropoxy-5-methyl-4-(4-piperidinyl)phenyl]-N⁴-[2-(isopropylsulfonyl)phenyl]-2,4-pyrimidinediamine. The chemical name of alectinib is 9-ethyl-6,6-dimethyl-8-(4-morpholinopiperidin-1-yl)-11-oxo-6,11-dihydro-5H-benzo[b]carbazole-3-carbonitrile. The chemical name of brigatinib is 5-Chloro-N²-{4-[4-(dimethylamino)-1-piperidinyl]-2-methoxyphenyl}-N⁴-[2-(dimethylphosphoryl)phenyl]-2,4-pyrimidinediamine. The chemical name of entrectinib is N-(5-(3,5-difluorobenzyl)-1H-indazol-3-yl)-4-(4-methylpiperazin-1-yl)-2-((tetrahydro-2H-pyran-4-yl)amino)benzamide. The chemical name of PF-06463922 is (10R)-7-Amino-12-fluoro-2,10,16-trimethyl-15-oxo-10,15,16,17-tetrahydro-2H-8,4-(metheno)pyrazolo[4,3-h][2,5,11]-benzoxadiazacyclotetradecine-3-carbonitrile. The chemical structure of CEP-37440 is (S)-2-((5-chloro-2-((6-(4-(2-hydroxyethyl)piperazin-1-yl)-1-methoxy-6,7,8,9-tetrahydro-5H-benzo[7]annulen-2-yl)amino)pyrimidin-4-yl)amino)-N-methylbenzamide. The chemical name of X-396 is (R)-6-amino-5-(1-(2,6-dichloro-3-fluorophenyl)ethoxy)-N-(4-(4-methylpiperazine-1-carbonyl)phenyl)pyridazine-3-carboxamide.

Drugs that inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993) can also be used. In a further aspect, the cell compositions of the present invention may be administered to a patient in conjunction with (e.g., before, simultaneously or following) bone marrow transplantation, T cell ablative therapy using chemotherapy agents such as, fludarabine, external-beam radiation therapy (XRT), cyclophosphamide, and/or antibodies such as OKT3 or CAMPATH. In one aspect, the cell compositions of the present invention are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

In embodiments, a CAR-expressing cell described herein is administered to a subject in combination with an indoleamine 2,3-dioxygenase (IDO) inhibitor. IDO is an enzyme that catalyzes the degradation of the amino acid, L-tryptophan, to kynurenine. Many cancers overexpress IDO, e.g., prostatic, colorectal, pancreatic, cervical, gastric, ovarian, head, and lung cancer. pDCs, macrophages, and dendritic cells (DCs) can express IDO. Without being bound by theory, it is thought that a decrease in L-tryptophan (e.g., catalyzed by IDO) results in an immunosuppressive milieu by inducing T-cell anergy and apoptosis. Thus, without being bound by theory, it is thought that an IDO inhibitor can enhance the efficacy of a CAR-expressing cell described herein, e.g., by decreasing the suppression or death of a CAR-expressing immune cell. In embodiments, the subject has a solid tumor, e.g., a solid tumor described herein, e.g., prostatic, colorectal, pancreatic, cervical, gastric, ovarian, head, or lung cancer. Exemplary inhibitors of IDO include but are not limited to 1-methyl-tryptophan, indoximod (NewLink Genetics) (see, e.g., Clinical Trial Identifier Nos. NCT01191216; NCT01792050), and INCB024360 (Incyte Corp.) (see, e.g., Clinical Trial Identifier Nos. NCT01604889; NCT01685255)

In embodiments, a CAR-expressing cell described herein is administered to a subject in combination with a modulator of myeloid-derived suppressor cells (MDSCs). MDSCs accumulate in the periphery and at the tumor site of many solid tumors. These cells suppress T cell responses, thereby hindering the efficacy of CAR-expressing cell therapy. Without being bound by theory, it is thought that administration of a MDSC modulator enhances the efficacy of a CAR-expressing cell described herein. In an embodiment, the subject has a solid tumor, e.g., a solid tumor described herein, e.g., glioblastoma. Exemplary modulators of MDSCs include but are not limited to MCS110 and BLZ945. MCS110 is a monoclonal antibody (mAb) against macrophage colony-stimulating factor (M-CSF). See, e.g., Clinical Trial Identifier No. NCT00757757. BLZ945 is a small molecule inhibitor of colony stimulating factor 1 receptor (CSF1R). See, e.g., Pyonteck et al. Nat. Med. 19(2013):1264-72. The structure of BLZ945 is shown below.

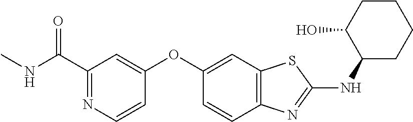 [SMILES: CNC(=O)c1cc(Oc2ccc3nc(N[C@@H]4CCCC[C@H]4O)sc3c2)ccn1]

In embodiments, a CAR-expressing cell described herein is administered to a subject in combination with an agent that inhibits or reduces the activity of immunosuppressive plasma cells. Immunosuppressive plasma cells have been shown to impede T cell-dependent immunogenic chemotherapy, such as oxaliplatin (Shalapour et al., Nature 2015, 521:94-101). In an embodiment, immunosuppressive plasma cells can express one or more of IgA, interleukin (IL)-10, and PD-L1. In an embodiment, the agent is a CD19 CAR-expressing cell or a BCMA CAR-expressing cell.

In some embodiments, a CAR-expressing cell described herein is administered to a subject in combination with a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15 (Admune Therapeutics, LLC). hetIL-15 is a heterodimeric non-covalent complex of IL-15 and IL-15Ra. hetIL-15 is described in, e.g., U.S. Pat. No. 8,124,084, U.S. 2012/0177598, U.S. 2009/0082299, U.S. 2012/0141413, and U.S. 2011/0081311, incorporated herein by reference. In embodiments, het-IL-15 is administered subcutaneously. In embodiments, the subject has a cancer, e.g., solid cancer, e.g., melanoma or colon cancer. In embodiments, the subject has a metastatic cancer.

In embodiments, a subject having a disease described herein, e.g., a hematological disorder, e.g., AML or MDS, is administered a CAR-expressing cell described herein in combination with an agent, e.g., cytotoxic or chemotherapy agent, a biologic therapy (e.g., antibody, e.g., monoclonal antibody, or cellular therapy), or an inhibitor (e.g., kinase inhibitor). In embodiments, the subject is administered a CAR-expressing cell described herein in combination with a cytotoxic agent, e.g., CPX-351 (Celator Pharmaceuticals), cytarabine, daunorubicin, vosaroxin (Sunesis Pharmaceuticals), sapacitabine (Cyclacel Pharmaceuticals), idarubicin, or mitoxantrone. CPX-351 is a liposomal formulation comprising cytarabine and daunorubicin at a 5:1 molar ratio. In embodiments, the subject is administered a CAR-expressing cell described herein in combination with a hypomethylating agent, e.g., a DNA methyltransferase inhibitor, e.g., azacitidine or decitabine. In embodiments, the subject is administered a CAR-expressing cell described herein in combination with a biologic therapy, e.g., an antibody or cellular therapy, e.g., 225Ac-lintuzumab (Actimab-A; Actinium Pharmaceuticals), IPH2102 (Innate Pharma/Bristol Myers Squibb), SGN-CD33A (Seattle Genetics), or gemtuzumab ozogamicin (Mylotarg; Pfizer). SGN-CD33A is an antibody-drug conjugate (ADC) comprising a pyrrolobenzodiazepine dimer that is attached to an anti-CD33 antibody. Actimab-A is an anti-CD33 antibody (lintuzumab) labeled with actinium. IPH2102 is a monoclonal antibody that targets killer immunoglobulin-like receptors (KIRs). In embodiments, the subject is administered a CAR-expressing cell described herein in combination a FLT3 inhibitor, e.g., sorafenib (Bayer), midostaurin (Novartis), quizartinib (Daiichi Sankyo), crenolanib (Arog Pharmaceuticals), PLX3397 (Daiichi Sankyo), AKN-028 (Akinion Pharmaceuticals), or ASP2215 (Astellas). In embodiments, the subject is administered a CAR-expressing cell described herein in combination with an isocitrate dehydrogenase (IDH) inhibitor, e.g., AG-221 (Celgene/Agios) or AG-120 (Agios/Celgene). In embodiments, the subject is administered a CAR-expressing cell described herein in combination with a cell cycle regulator, e.g., inhibitor of polo-like kinase 1 (Plk1), e.g., volasertib (Boehringer Ingelheim); or an inhibitor of cyclin-dependent kinase 9 (Cdk9), e.g., alvocidib (Tolero Pharmaceuticals/Sanofi Aventis). In embodiments, the subject is administered a CAR-expressing cell described herein in combination with a B cell receptor signaling network inhibitor, e.g., an inihibitor of B-cell lymphoma 2 (Bc1-2), e.g., venetoclax (Abbvie/Roche); or an inhibitor of Bruton's tyrosine kinase (Btk), e.g., ibrutinib (Pharmacyclics/Johnson & Johnson Janssen Pharmaceutical). In embodiments, the subject is administered a CAR-expressing cell described herein in combination with an inhibitor of M1 aminopeptidase, e.g., tosedostat (CTI BioPharmaNernalis); an inhibitor of histone deacetylase (HDAC), e.g., pracinostat (MEI Pharma); a multi-kinase inhibitor, e.g., rigosertib (Onconova Therapeutics/Baxter/SymBio); or a peptidic CXCR4 inverse agonist, e.g., BL-8040 (BioLineRx). In embodiments, the subject is administered a CD123-targeting CAR-expressing cell in combination with a CAR-expressing cell that targets an antigen other than CD123, e.g., CLL-1, BCMA, CD33, CD19, FLT-3, or folate receptor beta.

In another embodiment, the subjects receive an infusion of the CD123 CAR-expressing cell compositions of the present invention prior to transplantation, e.g., allogeneic stem cell transplant, of cells. In a preferred embodiment, CD123-CAR expressing cells transiently express CD123 CAR, e.g., by electroporation of an mRNA CD123 CAR, whereby the expression of the CD123 is terminated prior to infusion of donor stem cells to avoid engraftment failure.

Some patients may experience allergic reactions to the compounds of the present invention and/or other anti-cancer agent(s) during or after administration; therefore, anti-allergic agents are often administered to minimize the risk of an allergic reaction. Suitable anti-allergic agents include corticosteroids, such as dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M-Prednisol® and Solu-Medrol®); antihistamines, such as diphenhydramine (e.g., Benadryl®), hydroxyzine, and cyproheptadine; and bronchodilators, such as the beta-adrenergic receptor agonists, albuterol (e.g., Proventil®), and terbutaline (Brethine®).

Some patients may experience nausea during and after administration of the compound of the present invention and/or other anti-cancer agent(s); therefore, anti-emetics are used in preventing nausea (upper stomach) and vomiting. Suitable anti-emetics include aprepitant (Emend®), ondansetron (Zofran®), granisetron HCl (Kytril®), lorazepam (Ativan®. dexamethasone (Decadron®), prochlorperazine (Compazine®), casopitant (Rezonic® and Zunrisa®), and combinations thereof.

Medication to alleviate the pain experienced during the treatment period is often prescribed to make the patient more comfortable. Common over-the-counter analgesics, such Tylenol®, are often used. However, opioid analgesic drugs such as hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), and fentanyl (e.g., Duragesic®) are also useful for moderate or severe pain.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. Suitable cytoprotective agents include Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid).

The structure of the active compounds identified by code numbers, generic or trade names may be taken from the actual edition of the standard compendium “The Merck Index” or from databases, e.g. Patents International (e.g. IMS World Publications).

The above-mentioned compounds, which can be used in combination with a compound of the present invention, can be prepared and administered as described in the art, such as in the documents cited above.

In one embodiment, the present invention provides pharmaceutical compositions comprising at least one compound of the present invention (e.g., a compound of the present invention) or a pharmaceutically acceptable salt thereof together with a pharmaceutically acceptable carrier suitable for administration to a human or animal subject, either alone or together with other anti-cancer agents.

In one embodiment, the present invention provides methods of treating human or animal subjects suffering from a cellular proliferative disease, such as cancer. The present invention provides methods of treating a human or animal subject in need of such treatment, comprising administering to the subject a therapeutically effective amount of a compound of the present invention (e.g., a compound of the present invention) or a pharmaceutically acceptable salt thereof, either alone or in combination with other anti-cancer agents.

In particular, compositions will either be formulated together as a combination therapeutic or administered separately.

In combination therapy, the compound of the present invention and other anti-cancer agent(s) may be administered either simultaneously, concurrently or sequentially with no specific time limits, wherein such administration provides therapeutically effective levels of the two compounds in the body of the patient.

In a preferred embodiment, the compound of the present invention and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present invention and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another aspect of the present invention, kits that include one or more compound of the present invention and a combination partner as disclosed herein are provided. Representative kits include (a) a compound of the present invention or a pharmaceutically acceptable salt thereof, (b) at least one combination partner, e.g., as indicated above, whereby such kit may comprise a package insert or other labeling including directions for administration.

A compound of the present invention may also be used to advantage in combination with known therapeutic processes, for example, the administration of hormones or especially radiation. A compound of the present invention may in particular be used as a radiosensitizer, especially for the treatment of tumors which exhibit poor sensitivity to radiotherapy.

In one embodiment, the subject can be administered an agent which reduces or ameliorates a side effect associated with the administration of a CAR-expressing cell. Side effects associated with the administration of a CAR-expressing cell include, but are not limited to CRS, and hemophagocytic lymphohistiocytosis (HLH), also termed Macrophage Activation Syndrome (MAS). Symptoms of CRS include high fevers, nausea, transient hypotension, hypoxia, and the like. CRS may include clinical constitutional signs and symptoms such as fever, fatigue, anorexia, myalgias, arthalgias, nausea, vomiting, and headache. CRS may include clinical skin signs and symptoms such as rash. CRS may include clinical gastrointestinal signs and symsptoms such as nausea, vomiting and diarrhea. CRS may include clinical respiratory signs and symptoms such as tachypnea and hypoxemia. CRS may include clinical cardiovascular signs and symptoms such as tachycardia, widened pulse pressure, hypotension, increased cardac output (early) and potentially diminished cardiac output (late). CRS may include clinical coagulation signs and symptoms such as elevated d-dimer, hypofibrinogenemia with or without bleeding. CRS may include clinical renal signs and symptoms such as azotemia. CRS may include clinical hepatic signs and symptoms such as transaminitis and hyperbilirubinemia. CRS may include clinical neurologic signs and symptoms such as headache, mental status changes, confusion, delirium, word finding difficulty or frank aphasia, hallucinations, tremor, dymetria, altered gait, and seizures.

Accordingly, the methods described herein can comprise administering a CAR-expressing cell described herein to a subject and further administering one or more agents to manage elevated levels of a soluble factor resulting from treatment with a CAR-expressing cell. In one embodiment, the soluble factor elevated in the subject is one or more of IFN-γ, TNFα, IL-2 and IL-6. In an embodiment, the factor elevated in the subject is one or more of IL-1, GM-CSF, IL-10, IL-8, IL-5 and fraktalkine. Therefore, an agent administered to treat this side effect can be an agent that neutralizes one or more of these soluble factors. In one embodiment, the agent that neutralizes one or more of these soluble forms is an antibody or antigen binding fragment thereof. Examples of such agents include, but are not limited to a steroid (e.g., corticosteroid), an inhibitor of TNFα, and an inhibitor of IL-6. An example of a TNFα inhibitor is an anti-TNFα antibody molecule such as, infliximab, adalimumab, certolizumab pegol, and golimumab. Another example of a TNFα inhibitor is a fusion protein such as entanercept. Small molecule inhibitors of TNFα include, but are not limited to, xanthine derivatives (e.g. pentoxifylline) and bupropion. An example of an IL-6 inhibitor is an anti-IL-6 antibody molecule such as tocilizumab (toc), sarilumab, elsilimomab, CNTO 328, ALD518/BMS-945429, CNTO 136, CPSI-2364, CDP6038, VX30, ARGX-109, FE301, and FM101. In one embodiment, the anti-IL-6 antibody molecule is tocilizumab. An example of an IL-1R based inhibitor is anakinra.

In some embodiment, the subject is administered a corticosteroid, such as, e.g., methylprednisolone, hydrocortisone, among others.

In some embodiments, the subject is administered a vasopressor, such as, e.g., norepinephrine, dopamine, phenylephrine, epinephrine, vasopres sin, or a combination thereof.

In an embodiment, the subject can be administered an antipyretic agent. In an embodiment, the subject can be administered an analgesic agent.

In one embodiment, the subject can be administered an agent which enhances the activity of a CAR-expressing cell. For example, in one embodiment, the agent can be an agent which inhibits an inhibitory molecule, e.g., the agent is a checkpoint inhibitor. Inhibitory molecules, e.g., Programmed Death 1 (PD1), can, in some embodiments, decrease the ability of a CAR-expressing cell to mount an immune effector response. Examples of inhibitory molecules include PD1, PD-L1, CTLA4, TIM3, LAGS, VISTA, BTLA, TIGIT, LAIR1, CD160, 2B4 and TGF beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used to inhibit expression of an inhibitory molecule in the CAR-expressing cell. In an embodiment the inhibitor is an shRNA. In an embodiment, the inhibitory molecule is inhibited within a CAR-expressing cell. In these embodiments, a dsRNA molecule that inhibits expression of the inhibitory molecule is linked to the nucleic acid that encodes a component, e.g., all of the components, of the CAR.

In an embodiment, a nucleic acid molecule that encodes a dsRNA molecule that inhibits expression of the molecule that modulates or regulates, e.g., inhibits, T-cell function is operably linked to a promoter, e.g., a H1- or a U6-derived promoter such that the dsRNA molecule that inhibits expression of the molecule that modulates or regulates, e.g., inhibits, T-cell function is expressed, e.g., is expressed within a CAR-expressing cell. See e.g., Tiscornia G., “Development of Lentiviral Vectors Expressing siRNA,” Chapter 3, in Gene Transfer: Delivery and Expression of DNA and RNA (eds. Friedmann and Rossi). Cold Spring Harbor Laboratory Press, Cold Spring Harbor, N.Y., USA, 2007; Brummelkamp T R, et al. (2002) Science 296: 550-553; Miyagishi M, et al. (2002) Nat. Biotechnol. 19: 497-500. In an embodiment the nucleic acid molecule that encodes a dsRNA molecule that inhibits expression of the molecule that modulates or regulates, e.g., inhibits, T-cell function is present on the same vector, e.g., a lentiviral vector, that comprises a nucleic acid molecule that encodes a component, e.g., all of the components, of the CAR. In such an embodiment, the nucleic acid molecule that encodes a dsRNA molecule that inhibits expression of the molecule that modulates or regulates, e.g., inhibits, T-cell function is located on the vector, e.g., the lentiviral vector, 5′- or 3′- to the nucleic acid that encodes a component, e.g., all of the components, of the CAR. The nucleic acid molecule that encodes a dsRNA molecule that inhibits expression of the molecule that modulates or regulates, e.g., inhibits, T-cell function can be transcribed in the same or different direction as the nucleic acid that encodes a component, e.g., all of the components, of the CAR.

In an embodiment the nucleic acid molecule that encodes a dsRNA molecule that inhibits expression of the molecule that modulates or regulates, e.g., inhibits, T-cell function is present on a vector other than the vector that comprises a nucleic acid molecule that encodes a component, e.g., all of the components, of the CAR. In an embodiment, the nucleic acid molecule that encodes a dsRNA molecule that inhibits expression of the molecule that modulates or regulates, e.g., inhibits, T-cell function it transiently expressed within a CAR-expressing cell. In an embodiment, the nucleic acid molecule that encodes a dsRNA molecule that inhibits expression of the molecule that modulates or regulates, e.g., inhibits, T-cell function is stably integrated into the genome of a CAR-expressing cell.

Examples of dsRNA molecules useful for inhibiting expression of a molecule that modulates or regulates, e.g., inhibits, T-cell function, wherein the molecule that modulates or regulates, e.g., inhibits, T-cell function is PD-1 are provided below.

Provided in Table 18A below are the names of PDCD1 (PD1) RNAi agents (derived from their position in the mouse PDCD1 gene sequence NM_008798.2), along with the SEQ ID NOs: 216-263 representing the DNA sequence. Both sense (S) and antisense (AS) sequences are presented as 19mer and 21mer sequences are in this table. Also note that the position (PoS, e.g., 176) is derived from the position number in the mouse PDCD1 gene sequence NM_008798.2. SEQ ID NOs are indicated in groups of 12 that correspond with “sense 19” SEQ ID NOs: 608-619; “sense 21” SEQ ID NOs: 620-631; “asense 21” SEQ ID NOs: 632-643; “asense 19” SEQ ID NOs: 644-655.

TABLE 18A
Mouse PDCD1 (PD1) shRNA sequences

| Position | | | | | |
|---|---|---|---|---|---|
| on | Target | | | | |
| NM_008798.2 | region | Sense19 | Sense21 | Asense21 | Asense19 |
| 176 | CDS | GGAGGTCCC | CTGGAGGTC | TAGAAGGTG | TAGAAGGTG |
| | | TCACCTTCTA | CCTCACCTTC | AGGGACCTC | AGGGACCTC |
| | | (SEQ ID NO: | TA | CAG | C (SEQ ID NO: |
| | | 608) | (SEQ ID NO: | (SEQ ID NO: | 644) |
| | | | 620) | 632) | |
| 260 | CDS | CGGAGGATC | GTCGGAGGA | TTCAGCATA | TTCAGCATA |
| | | TTATGCTGA | TCTTATGCTG | AGATCCTCC | AGATCCTCCG |
| | | A (SEQ ID NO: | AA | GAC | (SEQ ID NO: |
| | | 609) | (SEQ ID NO: | (SEQ ID NO: | 645) |
| | | | 621) | 633) | |
| 359 | CDS | CCCGCTTCCA | TGCCCGCTT | TGTATGATCT | TGTATGATC |
| | | GATCATACA | CCAGATCAT | GGAAGCGGG | TGGAAGCGGG |
| | | (SEQ ID NO: | ACA | CA | (SEQ ID NO: |
| | | 610) | (SEQ ID NO: | (SEQ ID NO: | 646) |
| | | | 622) | 634) | |
| 528 | CDS | GGAGACCTC | CTGGAGACC | ATATCTTGTT | ATATCTTGTT |
| | | AACAAGATA | TCAACAAGA | GAGGTCTCC | GAGGTCTCC |
| | | T (SEQ ID NO: | TAT | AG | (SEQ ID NO: |
| | | 611) | (SEQ ID NO: | (SEQ ID NO: | 647) |
| | | | 623) | 635) | |
| 581 | CDS | AAGGCATGG | TCAAGGCAT | ATACCAATG | ATACCAATG |
| | | TCATTGGTAT | GGTCATTGG | ACCATGCCT | ACCATGCCTT |
| | | (SEQ ID NO: | TAT | TGA | (SEQ ID NO: |
| | | 612) | (SEQ ID NO: | (SEQ ID NO: | 648) |
| | | | 624) | 636) | |
| 584 | CDS | GCATGGTCA | AGGCATGGT | ATGATACCA | ATGATACCA |
| | | TTGGTATCA | CATTGGTAT | ATGACCATG | ATGACCATGC |
| | | T (SEQ ID NO: | CAT | CCT | (SEQ ID NO: |
| | | 613) | (SEQ ID NO: | (SEQ ID NO: | 649) |
| | | | 625) | 637) | |
| 588 | CDS | GGTCATTGG | ATGGTCATT | ATGGTCATT | ATGGTCATT |
| | | TATCATGAG | GGTATCATG | GGTATCATG | GGTATCATG |
| | | T (SEQ ID NO: | AGT | AGT | A (SEQ ID NO: |
| | | 614) | (SEQ ID NO: | (SEQ ID NO: | 650) |
| | | | 626) | 638) | |
| 609 | CDS | CCTAGTGGG | GCCCTAGTG | GCCCTAGTG | GCCCTAGTG |
| | | TATCCCTGT | GGTATCCCT | GGTATCCCT | GGTATCCCTG |
| | | A (SEQ ID NO: | GTA | GTA | (SEQ ID NO: |
| | | 615) | (SEQ ID NO: | (SEQ ID NO: | 651) |
| | | | 627) | 639) | |
| 919 | CDS | GAGGATGGA | ATGAGGATG | ATGAGGATG | ATGAGGATG |
| | | CATTGTTCTT | GACATTGTT | GACATTGTT | GACATTGTTC |
| | | (SEQ ID NO: | CTT | CTT | (SEQ ID NO: |
| | | 616) | (SEQ ID NO: | (SEQ ID NO: | 652) |
| | | | 628) | 640) | |
| 1021 | 3′UTR | GCATGCAGG | GAGCATGCA | GAGCATGCA | GAGCATGCA |
| | | CTACAGTTC | GGCTACAGT | GGCTACAGT | GGCTACAGTT |
| | | A (SEQ ID NO: | TCA | TCA | (SEQ ID NO: |
| | | 617) | (SEQ ID NO: | (SEQ ID NO: | 653) |
| | | | 629) | 641) | |
| 1097 | 3′UTR | CCAGCACAT | TTCCAGCAC | TTCCAGCAC | TTCCAGCAC |
| | | GCACTGTTG | ATGCACTGT | ATGCACTGT | ATGCACTGTT |
| | | A (SEQ ID NO: | TGA | TGA | (SEQ ID NO: |
| | | 618) | (SEQ ID NO: | (SEQ ID NO: | 654) |
| | | | 630) | 642) | |
| 1101 | 3′UTR | CACATGCAC | AGCACATGC | AGCACATGC | AGCACATGC |
| | | TGTTGAGTG | ACTGTTGAG | ACTGTTGAG | ACTGTTGAGT |
| | | A (SEQ ID NO: | TGA | TGA | (SEQ ID NO: |
| | | 619) | (SEQ ID NO: | (SEQ ID NO: | 655) |
| | | | 631) | 643) | |

Provided in Table 19A below are the names of PDCD1 (PD1) RNAi agents (derived from their position in the human PDCD1 gene sequence, along with the SEQ ID NOs. 264-311 representing the DNA sequence. Both sense (S) and antisense (AS) sequences are presented as 19mer and 21mer sequences. SEQ ID NOs are indicated in groups of 12 that correspond with “sense 19” SEQ ID NOs: 656-667; “sense 21” SEQ ID NOs: 668-679; “asense 21” SEQ ID NOs: 680-691; “asense 19” SEQ ID NOs: 692-703.

TABLE 19A
Human PDCD1 (PD1) shRNA sequences

| Position | | | | | |
|---|---|---|---|---|---|
| on | Target | | | | |
| NM_005018.2 | region | Sense19 | Asense19 | Sense21 | Asense21 |
| 145 | CDS | GGCCAGGATGG | TCTAAGAACCA | GCGGCCAGGAT | TCTAAGAACCA |
| | | TTCTTAGA (SEQ | TCCTGGCC | GGTTCTTAGA | TCCTGGCCGC |
| | | ID NO: 656) | (SEQ ID NO: 668) | (SEQ ID NO: 680) | (SEQ ID NO: 692) |
| 271 | CDS | GCTTCGTGCTA | TACCAGTTTAG | GAGCTTCGTGC | TACCAGTTTAG |
| | | AACTGGTA | CACGAAGC | TAAACTGGTA | CACGAAGCTC |
| | | (SEQ ID NO: 657) | (SEQ ID NO: 669) | (SEQ ID NO: 681) | (SEQ ID NO: 693) |
| 393 | CDS | GGGCGTGACTT | TCATGTGGAAG | ACGGGCGTGAC | TCATGTGGAAG |
| | | CCACATGA | TCACGCCC | TTCCACATGA | TCACGCCCGT |
| | | (SEQ ID NO: 658) | (SEQ ID NO: 670) | (SEQ ID NO: 682) | (SEQ ID NO: 694) |
| 1497 | 3′UTR | CAGGCCTAGAG | TGAAACTTCTC | TGCAGGCCTAG | TGAAACTTCTC |
| | | AAGTTTCA | TAGGCCTG | AGAAGTTTCA | TAGGCCTGCA |
| | | (SEQ ID NO: 659) | (SEQ ID NO: 671) | (SEQ ID NO: 683) | (SEQ ID NO: 695) |
| 1863 | 3′UTR | CTTGGAACCCA | TTCAGGAATGG | TCCTTGGAACC | TTCAGGAATGG |
| | | TTCCTGAA | GTTCCAAG | CATTCCTGAA | GTTCCAAGGA |
| | | (SEQ ID NO: 660) | (SEQ ID NO: 672) | (SEQ ID NO: 684) | (SEQ ID NO: 696) |
| 1866 | 3′UTR | GGAACCCATTC | AATTTCAGGAA | TTGGAACCCAT | AATTTCAGGAA |
| | | CTGAAATT | TGGGTTCC | TCCTGAAATT | TGGGTTCCAA |
| | | (SEQ ID NO: 661) | (SEQ ID NO: 673) | (SEQ ID NO: 685) | (SEQ ID NO: 697) |
| 1867 | 3′UTR | GAACCCATTCC | TAATTTCAGGA | TGGAACCCATT | TAATTTCAGGA |
| | | TGAAATTA | ATGGGTTC | CCTGAAATTA | ATGGGTTCCA |
| | | (SEQ ID NO: 662) | (SEQ ID NO: 674) | (SEQ ID NO: 686) | (SEQ ID NO: 698) |
| 1868 | 3′UTR | AACCCATTCCT | ATAATTTCAGG | GGAACCCATTC | ATAATTTCAGG |
| | | GAAATTAT | AATGGGTT | CTGAAATTAT | AATGGGTTCC |
| | | (SEQ ID NO: 663) | (SEQ ID NO: 675) | (SEQ ID NO: 687) | (SEQ ID NO: 699) |
| 1869 | 3′UTR | ACCCATTCCTG | AATAATTTCAG | GAACCCATTCC | AATAATTTCAG |
| | | AAATTATT | GAATGGGT | TGAAATTATT | GAATGGGTTC |
| | | (SEQ ID NO: 664) | (SEQ ID NO: 676) | (SEQ ID NO: 688) | (SEQ ID NO: 700) |
| 1870 | 3′UTR | CCCATTCCTGA | AAATAATTTCA | AACCCATTCCT | AAATAATTTCA |
| | | AATTATTT | GGAATGGG | GAAATTATTT | GGAATGGGTT |
| | | (SEQ ID NO: 665) | (SEQ ID NO: 677) | (SEQ ID NO: 689) | (SEQ ID NO: 701) |
| 2079 | 3′UTR | CTGTGGTTCTAT | TAATATAATAG | CCCTGTGGTTCT | TAATATAATAG |
| | | TATATTA | AACCACAG | ATTATATTA | AACCACAGGG |
| | | (SEQ ID NO: 666) | (SEQ ID NO: 678) | (SEQ ID NO: 690) | (SEQ ID NO: 702) |
| 2109 | 3′UTR | AAATATGAGAG | TTAGCATGCTC | TTAAATATGAG | TTAGCATGCTC |
| | | CATGCTAA | TCATATTT | AGCATGCTAA | TCATATTTAA |
| | | (SEQ ID NO: 667) | (SEQ ID NO: 679) | (SEQ ID NO: 691) | (SEQ ID NO: 703) |

In one embodiment, the inhibitor of an inhibitory signal can be, e.g., an antibody or antibody fragment that binds to an inhibitory molecule. For example, the agent can be an antibody or antibody fragment that binds to PD1, PD-L1, PD-L2 or CTLA4 (e.g., ipilimumab (also referred to as MDX-010 and MDX-101, and marketed as Yervoy®; Bristol-Myers Squibb; Tremelimumab (IgG2 monoclonal antibody available from Pfizer, formerly known as ticilimumab, CP-675,206).). In an embodiment, the agent is an antibody or antibody fragment that binds to TIM3. In an embodiment, the agent is an antibody or antibody fragment that binds to LAG3. In embodiments, the agent that enhances the activity of a CAR-expressing cell, e.g., inhibitor of an inhibitory molecule, is administered in combination with an allogeneic CAR, e.g., an allogeneic CAR described herein (e.g., described in the Allogeneic CAR section herein).

PD-1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD-1, PD-L1 and PD-L2 have been shown to downregulate T cell activation upon binding to PD-1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD-1 with PD-L1.

Antibodies, antibody fragments, and other inhibitors of PD-1, PD-L1 and PD-L2 are available in the art and may be used combination with a cars of the present invention described herein. For example, nivolumab (also referred to as BMS-936558 or MDX1106; Bristol-Myers Squibb) is a fully human IgG4 monoclonal antibody which specifically blocks PD-1. Nivolumab (clone 5C4) and other human monoclonal antibodies that specifically bind to PD-1 are disclosed in U.S. Pat. No. 8,008,449 and WO2006/121168. Pidilizumab (CT-011; Cure Tech) is a humanized IgG1k monoclonal antibody that binds to PD-1. Pidilizumab and other humanized anti-PD-1 monoclonal antibodies are disclosed in WO2009/101611. Pembrolizumab (formerly known as lambrolizumab, and also referred to as MK03475; Merck) is a humanized IgG4 monoclonal antibody that binds to PD-1. Pembrolizumab and other humanized anti-PD-1 antibodies are disclosed in U.S. Pat. No. 8,354,509 and WO2009/114335. MEDI4736 (Medimmune) is a human monoclonal antibody that binds to PDL1, and inhibits interaction of the ligand with PD1. MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No.: 20120039906. Other anti-PD-L1 binding agents include YW243.55.570 (heavy and light chain variable regions are shown in SEQ ID NOs 20 and 21 in WO2010/077634) and MDX-1 105 (also referred to as BMS-936559, and, e.g., anti-PD-L1 binding agents disclosed in WO2007/005874). AMP-224 (B7-DCIg; Amplimmune; e.g., disclosed in WO2010/027827 and WO2011/066342), is a PD-L2 Fc fusion soluble receptor that blocks the interaction between PD-1 and B7-H1. Other anti-PD-1 antibodies include AMP 514 (Amplimmune), among others, e.g., anti-PD-1 antibodies disclosed in U.S. Pat. No. 8,609,089, US 2010028330, and/or US 20120114649.

In one embodiment, the anti-PD-1 antibody or fragment thereof is an anti-PD-1 antibody molecule as described in US 2015/0210769, entitled “Antibody Molecules to PD-1 and Uses Thereof,” incorporated by reference in its entirety. In one embodiment, the anti-PD-1 antibody molecule includes at least one, two, three, four, five or six CDRs (or collectively all of the CDRs) from a heavy and light chain variable region from an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1, or a sequence substantially identical (e.g., at least 80%, 85%, 90%, 92%, 95%, 97%, 98%, 99% or higher identical) to any of the aforesaid sequences; or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-PD-1 antibody molecule comprises at least one, two, three or four variable regions from an antibody described herein, e.g., an antibody chosen from any of BAP049-hum01, BAP049-hum02, BAP049-hum03, BAP049-hum04, BAP049-hum05, BAP049-hum06, BAP049-hum07, BAP049-hum08, BAP049-hum09, BAP049-hum10, BAP049-hum11, BAP049-hum12, BAP049-hum13, BAP049-hum14, BAP049-hum15, BAP049-hum16, BAP049-Clone-A, BAP049-Clone-B, BAP049-Clone-C, BAP049-Clone-D, or BAP049-Clone-E; or as described in Table 1 of US 2015/0210769, or encoded by the nucleotide sequence in Table 1; or a sequence substantially identical (e.g., at least 80%, 85%, 90%, 92%, 95%, 97%, 98%, 99% or higher identical) to any of the aforesaid sequences.

TIM3 (T cell immunoglobulin-3) also negatively regulates T cell function, particularly in IFN-g-secreting CD4+ T helper 1 and CD8+ T cytotoxic 1 cells, and plays a critical role in T cell exhaustion. Inhibition of the interaction between TIM3 and its ligands, e.g., galectin-9 (Ga19), phosphotidylserine (PS), and HMGB1, can increase immune response. Antibodies, antibody fragments, and other inhibitors of TIM3 and its ligands are available in the art and may be used combination with a CD19 CAR described herein. For example, antibodies, antibody fragments, small molecules, or peptide inhibitors that target TIM3 binds to the IgV domain of TIM3 to inhibit interaction with its ligands. Antibodies and peptides that inhibit TIM3 are disclosed in WO2013/006490 and US20100247521. Other anti-TIM3 antibodies include humanized versions of RMT3-23 (disclosed in Ngiow et al., 2011, Cancer Res, 71:3540-3551), and clone 8B.2C12 (disclosed in Monney et al., 2002, Nature, 415:536-541). Bi-specific antibodies that inhibit TIM3 and PD-1 are disclosed in US20130156774.

In one embodiment, the anti-TIM3 antibody or fragment thereof is an anti-TIM3 antibody molecule as described in US 2015/0218274, entitled “Antibody Molecules to TIM3 and Uses Thereof,” incorporated by reference in its entirety. In one embodiment, the anti-TIM3 antibody molecule includes at least one, two, three, four, five or six CDRs (or collectively all of the CDRs) from a heavy and light chain variable region from an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences, or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-TIM3 antibody molecule comprises at least one, two, three or four variable regions from an antibody described herein, e.g., an antibody chosen from any of ABTIM3, ABTIM3-hum01, ABTIM3-hum02, ABTIM3-hum03, ABTIM3-hum04, ABTIM3-hum05, ABTIM3-hum06, ABTIM3-hum07, ABTIM3-hum08, ABTIM3-hum09, ABTIM3-hum10, ABTIM3-hum11, ABTIM3-hum12, ABTIM3-hum13, ABTIM3-hum14, ABTIM3-hum15, ABTIM3-hum16, ABTIM3-hum17, ABTIM3-hum18, ABTIM3-hum19, ABTIM3-hum20, ABTIM3-hum21, ABTIM3-hum22, ABTIM3-hum23; or as described in Tables 1-4 of US 2015/0218274; or encoded by the nucleotide sequence in Tables 1-4; or a sequence substantially identical (e.g., at least 80%, 85%, 90%, 92%, 95%, 97%, 98%, 99% or higher identical) to any of the aforesaid sequences

In other embodiments, the agent that enhances the activity of a CAR-expressing cell is a CEACAM inhibitor (e.g., CEACAM-1, CEACAM-3, and/or CEACAM-5 inhibitor). In one embodiment, the inhibitor of CEACAM is an anti-CEACAM antibody molecule. Exemplary anti-CEACAM-1 antibodies are described in WO 2010/125571, WO 2013/082366 WO 2014/059251 and WO 2014/022332, e.g., a monoclonal antibody 34B1, 26H7, and 5F4; or a recombinant form thereof, as described in, e.g., US 2004/0047858, U.S. Pat. No. 7,132,255 and WO 99/052552. In other embodiments, the anti-CEACAM antibody binds to CEACAM-5 as described in, e.g., Zheng et al. PLoS One. 2010 Sep. 2; 5(9). pii: e12529 (DOI:10:1371/journal.pone.0021146), or crossreacts with CEACAM-1 and CEACAM-5 as described in, e.g., WO 2013/054331 and US 2014/0271618.

Without wishing to be bound by theory, carcinoembryonic antigen cell adhesion molecules (CEACAM), such as CEACAM-1 and CEACAM-5, are believed to mediate, at least in part, inhibition of an anti-tumor immune response (see e.g., Markel et al. J Immunol. 2002 Mar. 15; 168(6):2803-10; Markel et al. J Immunol. 2006 Nov. 1; 177(9):6062-71; Markel et al. Immunology. 2009 February; 126(2):186-200; Markel et al. Cancer Immunol Immunother. 2010 February; 59(2):215-30; Ortenberg et al. Mol Cancer Ther. 2012 June; 11(6):1300-10; Stern et al. J Immunol. 2005 Jun. 1; 174(11):6692-701; Zheng et al. PLoS One. 2010 Sep. 2; 5(9). pii: e12529). For example, CEACAM-1 has been described as a heterophilic ligand for TIM-3 and as playing a role in TIM-3-mediated T cell tolerance and exhaustion (see e.g., WO 2014/022332; Huang, et al. (2014) Nature doi:10.1038/nature13848). In embodiments, co-blockade of CEACAM-1 and TIM-3 has been shown to enhance an anti-tumor immune response in xenograft colorectal cancer models (see e.g., WO 2014/022332; Huang, et al. (2014), supra). In other embodiments, co-blockade of CEACAM-1 and PD-1 reduce T cell tolerance as described, e.g., in WO 2014/059251. Thus, CEACAM inhibitors can be used with the other immunomodulators described herein (e.g., anti-PD-1 and/or anti-TIM-3 inhibitors) to enhance an immune response against a cancer, e.g., a melanoma, a lung cancer (e.g., NSCLC), a bladder cancer, a colon cancer an ovarian cancer, and other cancers as described herein.

LAG3 (lymphocyte activation gene-3 or CD223) is a cell surface molecule expressed on activated T cells and B cells that has been shown to play a role in CD8+ T cell exhaustion. Antibodies, antibody fragments, and other inhibitors of LAG3 and its ligands are available in the art and may be used combination with a CD19 CAR described herein. For example, BMS-986016 (Bristol-Myers Squib) is a monoclonal antibody that targets LAG3. IMP701 (Immutep) is an antagonist LAG3 antibody and IMP731 (Immutep and GlaxoSmithKline) is a depleting LAG3 antibody. Other LAG3 inhibitors include IMP321 (Immutep), which is a recombinant fusion protein of a soluble portion of LAG3 and Ig that binds to MHC class II molecules and activates antigen presenting cells (APC). Other antibodies are disclosed, e.g., in WO2010/019570.

In one embodiment, the anti-LAG3 antibody or fragment thereof is an anti-LAG3 antibody molecule as described in US 2015/0259420, entitled “Antibody Molecules to LAG3 and Uses Thereof,” incorporated by reference in its entirety.

In one embodiment, the anti-LAG3 antibody molecule includes at least one, two, three, four, five or six CDRs (or collectively all of the CDRs) from a heavy and light chain variable region from an antibody chosen from any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050(Ser) (e.g., BAP050-hum01-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420; or encoded by the nucleotide sequence in Table 1; or a sequence substantially identical (e.g., at least 80%, 85%, 90%, 92%, 95%, 97%, 98%, 99% or higher identical) to any of the aforesaid sequences, or closely related CDRs, e.g., CDRs which are identical or which have at least one amino acid alteration, but not more than two, three or four alterations (e.g., substitutions, deletions, or insertions, e.g., conservative substitutions).

In yet another embodiment, the anti-LAG3 antibody molecule comprises at least one, two, three or four variable regions from an antibody described herein, e.g., an antibody chosen from any of BAP050-hum01, BAP050-hum02, BAP050-hum03, BAP050-hum04, BAP050-hum05, BAP050-hum06, BAP050-hum07, BAP050-hum08, BAP050-hum09, BAP050-hum10, BAP050-hum11, BAP050-hum12, BAP050-hum13, BAP050-hum14, BAP050-hum15, BAP050-hum16, BAP050-hum17, BAP050-hum18, BAP050-hum19, BAP050-hum20, huBAP050(Ser) (e.g., BAP050-hum01-Ser, BAP050-hum02-Ser, BAP050-hum03-Ser, BAP050-hum04-Ser, BAP050-hum05-Ser, BAP050-hum06-Ser, BAP050-hum07-Ser, BAP050-hum08-Ser, BAP050-hum09-Ser, BAP050-hum10-Ser, BAP050-hum11-Ser, BAP050-hum12-Ser, BAP050-hum13-Ser, BAP050-hum14-Ser, BAP050-hum15-Ser, BAP050-hum18-Ser, BAP050-hum19-Ser, or BAP050-hum20-Ser), BAP050-Clone-F, BAP050-Clone-G, BAP050-Clone-H, BAP050-Clone-I, or BAP050-Clone-J; or as described in Table 1 of US 2015/0259420; or encoded by the nucleotide sequence in Tables 1; or a sequence substantially identical (e.g., at least 80%, 85%, 90%, 92%, 95%, 97%, 98%, 99% or higher identical) to any of the aforesaid sequences.

In some embodiments, the agent which enhances the activity of a CAR-expressing cell can be, e.g., a fusion protein comprising a first domain and a second domain, wherein the first domain is an inhibitory molecule, or fragment thereof, and the second domain is a polypeptide that is associated with a positive signal, e.g., a polypeptide comrpsing an antracellular signaling domain as described herein. In some embodiments, the polypeptide that is associated with a positive signal can include a costimulatory domain of CD28, CD27, ICOS, e.g., an intracellular signaling domain of CD28, CD27 and/or ICOS, and/or a primary signaling domain, e.g., of CD3 zeta, e.g., described herein. In one embodiment, the fusion protein is expressed by the same cell that expressed the CAR. In another embodiment, the fusion protein is expressed by a cell, e.g., a T cell that does not express a CD123 CAR.

In one embodiment, the agent which enhances activity of a CAR-expressing cell described herein is miR-17-92.

In one embodiment, the agent which enhances activity of a CAR-described herein is a cytokine. Cytokines have important functions related to T cell expansion, differentiation, survival, and homeostatis. Cytokines that can be administered to the subject receiving a CAR-expressing cell described herein include: IL-2, IL-4, IL-7, IL-9, IL-15, IL-18, and IL-21, or a combination thereof. In preferred embodiments, the cytokine administered is IL-7, IL-15, or IL-21, or a combination thereof. The cytokine can be administered once a day or more than once a day, e.g., twice a day, three times a day, or four times a day. The cytokine can be administered for more than one day, e.g. the cytokine is administered for 2 days, 3 days, 4 days, 5 days, 6 days, 1 week, 2 weeks, 3 weeks, or 4 weeks. For example, the cytokine is administered once a day for 7 days.

In embodiments, the cytokine is administered in combination with CAR-expressing T cells. The cytokine can be administered simultaneously or concurrently with the CAR-expressing T cells, e.g., administered on the same day. The cytokine may be prepared in the same pharmaceutical composition as the CAR-expressing T cells, or may be prepared in a separate pharmaceutical composition. Alternatively, the cytokine can be administered shortly after administration of the CAR-expressing T cells, e.g., 1 day, 2 days, 3 days, 4 days, 5 days, 6 days, or 7 days after administration of the CAR-expressing T cells. In embodiments where the cytokine is administered in a dosing regimen that occurs over more than one day, the first day of the cytokine dosing regimen can be on the same day as administration with the CAR-expressing T cells, or the first day of the cytokine dosing regimen can be 1 day, 2 days, 3 days, 4 days, 5 days, 6 days, or 7 days after administration of the CAR-expressing T cells. In one embodiment, on the first day, the CAR-expressing T cells are administered to the subject, and on the second day, a cytokine is administered once a day for the next 7 days. In a preferred embodiment, the cytokine to be administered in combination with CAR-expressing T cells is IL-7, IL-15, or IL-21.

In other embodiments, the cytokine is administered a period of time after administration of CAR-expressing cells, e.g., at least 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of CAR-expressing cells. In one embodiment, the cytokine is administered after assessment of the subject's response to the CAR-expressing cells. For example, the subject is administered CAR-expressing cells according to the dosage and regimens described herein. The response of the subject to CAR-expressing cell therapy is assessed at 2 weeks, 3 weeks, 4 weeks, 6 weeks, 8 weeks, 10 weeks, 12 weeks, 4 months, 5 months, 6 months, 7 months, 8 months, 9 months, 10 months, 11 months, or 1 year or more after administration of CAR-expressing cells, using any of the methods described herein, including inhibition of tumor growth, reduction of circulating tumor cells, or tumor regression. Subjects that do not exhibit a sufficient response to CAR-expressing cell therapy can be administered a cytokine. Administration of the cytokine to the subject that has sub-optimal response to the CAR-expressing cell therapy improves CAR-expressing cell efficacy or anti-cancer activity. In a preferred embodiment, the cytokine administered after administration of CAR-expressing cells is IL-7.

Combination with CD19 Inhibitors

The methods and compositions disclosed herein can be used in combination with a CD19 inhibitor. In some embodiments, the CD123CAR-containing cells and the CD19 inhibitor (e.g., one or more cells that express a CAR molecule that binds CD19, e.g., a CAR molecule that binds CD19 described herein) are administered simultaneously or concurrently, or sequentially.

In some embodiments, the CD123CAR-containing cells and the CD19 inhibitor are infused into a subject simultaneously or concurrently, e.g., are admixed in the same infusion volume. For example, a population of CD123CAR-containing cells and CD19CAR-containing cells are mixed together. Alternatively, a population of cells co-expressing a CD123CAR and a CD19CAR is administered. In other embodiments, the simultaneous administration comprises separate administration of the CD123CAR-containing cells and the CD19 inhibitor, e.g., within a predetermined time interval (e.g., within 15, 30, or 45 minutes of each other).

In some embodiments, the start of the CD123CAR-containing cells and the start of the CD19 inhibitor are within 1, 2, 3, 4, 6, 12, 18, or 24 hours of each other, or within 1, 2, 3, 4, 5, 10, 15, 20, 25, 30, 35, 40, 60, 80, or 100 days of each other. In some embodiments, the end of the CD123CAR-containing cells delivery and the end of the CD19 inhibitor delivery are within 1, 2, 3, 4, 6, 12, 18, or 24 hours of each other, or within 1, 2, 3, 4, 5, 10, 15, 20, 25, 30, 35, 40, 60, 80, or 100 days of each other. In some embodiments, the overlap in terms of administration between the of the CD123CAR-containing cells delivery (e.g., infusion) and the end of CD19 inhibitor delivery (e.g., infusion) is at least 1, 2, 3, 4, 5, 10, 15, 20, 25, 30 minutes. In one embodiment, the CD19 inhibitor is administered prior to the CD123CAR-containing cells. In other embodiments, the CD123CAR-containing cells are administered prior to the CD19 inhibitor.

In some embodiments, the CD123CAR-containing cells are administered while the CD19 inhibitor (e.g., one or more cells that express a CD19CAR molecule) is present (e.g., cells undergoing expansion) in the subject. In other embodiments, the CD19 inhibitor (e.g., one or more cells that express a CD19CAR molecule) is administered while the CD123CAR-containing cells are present (e.g., cells undergoing expansion) in the subject.

A CD19 inhibitor includes, but is not limited to, a CD19 CAR-expressing cell, e.g., a CD19 CART cell, or an anti-CD19 antibody (e.g., an anti-CD19 mono- or bispecific antibody) or a fragment or conjugate thereof.

In one embodiment, a CAR-expressing cell described herein is administered to a subject in combination with a CD19 CAR-cell (e.g., CART cell) (e.g., CTL019, e.g., as described in WO2012/079000, incorporated herein by reference).

In other embodiments, the CAR-expressing cell described herein is administered to a subject in combination with a CD19 CAR-cell (e.g., CART cell) that includes a humanized antigen binding domain as described in WO2014/153270 (e.g., Table 3 of WO2014/153270), incorporated herein by reference.

The CD19 inhibitor (e.g., a first CD19 CAR-expressing cell) and a second CD123 CAR-expressing cell may be expressed by the same cell type or different types. For instance, in some embodiments, the cell expressing a CD19 CAR is a CD4+ T cell and the cell expressing a CD123 CAR is a CD8+ T cell, or the cell expressing a CD19 CAR is a CD8+ T cell and the cell expressing a CD123 CAR is a CD4+ T cell. In other embodiments, the cell expressing a CD19 CAR is a T cell and the cell expressing a CD123 CAR is a NK cell, or the cell expressing a CD19 CAR is a NK cell and the cell expressing a CD123 CAR is a T cell. In other embodiments, the cell expressing a CD19 CAR and the cell expressing a CD123 CAR are both NK cells or are both T cells, e.g., are both CD4+ T cells, or are both CD8+ T cells. In yet other embodiments, a single cell expresses the CD19 CAR and CD123 CAR, and this cell is, e.g., a NK cell or a T cell such as a CD4+ T cell or CD8+ T cell.

The first CAR and second CAR can comprise the same or different intracellular signaling domains. For instance, in some embodiments, the CD19 CAR comprises a CD3 zeta signaling domain and the CD123 CAR comprises a costimulatory domain, e.g., a 41BB, CD27 or CD28 costimulatory domain, while in some embodiments, the CD19 CAR comprises a costimulatory domain, e.g., a 41BB, CD27 or CD28 costimulatory domain and the CD123 CAR comprises a CD3 zeta signaling domain. In other embodiments, each of the CD19 CAR and the CD123 CAR comprises the same type of primary signaling domaine, e.g., a CD3 zeta signaling domain, but the CD19 CAR and the CD123 CAR comprise different costimulatory domains, e.g., (1) the CD19 CAR comprises a 41BB costimulatory domain and the CD123 CAR comprises a different costimulatory domain e.g., a CD27 costimulatory domain, (2) the CD19 CAR comprises a CD27 costimulatory domain and the CD123 CAR comprises a different costimulatory domain e.g., a 41BB costimulatory domain, (3) the CD19 CAR comprises a 41BB costimulatory domain and the CD123 CAR comprises a CD28 costimulatory domain, (4) the CD19 CAR comprises a CD28 costimulatory domain and the CD123 CAR comprises a different costimulatory domain e.g., a 41BB costimulatory domain, (5) the CD19 CAR comprises a CD27 costimulatory domain and the CD123 CAR comprises a CD28 costimulatory domain, or (6) the CD19 CAR comprises a CD28 costimulatory domain and the CD123 CAR comprises a CD27 costimulatory domain. In another embodiment, a cell comprises a CAR that comprises both a CD19 antigen-binding domain and a CD123 antigen-binding domain, e.g., a bispecific antibody.

In embodiments, the subject has acute myeloid leukemia (AML), e.g., a CD19 positive AML or a CD19 negative AML. In embodiments, the subject has a CD19+ lymphoma, e.g., a CD19+ Non-Hodgkin's Lymphoma (NHL), a CD19+FL, or a CD19+ DLBCL. In embodiments, the subject has a relapsed or refractory CD19+ lymphoma. In embodiments, a lymphodepleting chemotherapy is administered to the subject prior to, concurrently with, or after administration (e.g., infusion) of CD19 CART cells. In an example, the lymphodepleting chemotherapy is administered to the subject prior to administration of CD19 CART cells. For example, the lymphodepleting chemotherapy ends 1-4 days (e.g., 1, 2, 3, or 4 days) prior to CD19 CART cell infusion. In embodiments, multiple doses of CD19 CART cells are administered, e.g., as described herein. For example, a single dose comprises about 5×10⁸ CD19 CART cells. In embodiments, a lymphodepleting chemotherapy is administered to the subject prior to, concurrently with, or after administration (e.g., infusion) of a CAR-expressing cell described herein, e.g., a non-CD19 CAR-expresing cell. In embodiments, a CD19 CART is administered to the subject prior to, concurrently with, or after administration (e.g., infusion) of a non-CD19 CAR-expressing cell, e.g., a non-CD19 CAR-expressing cell described herein.

In some embodiments, a CAR-expressing cell described herein is administered to a subject in combination with a CD19 CAR-expressing cell, e.g., CTL019, e.g., as described in WO2012/079000, incorporated herein by reference, for treatment of a disease associated with the expression of CD123, e.g., a cancer described herein. Without being bound by theory, it is believed that administering a CD19 CAR-expressing cell in combination with a CAR-expressing cell improves the efficacy of a CAR-expressing cell described herein by targeting early lineage cancer cells, e.g., cancer stem cells, modulating the immune response, depleting regulatory B cells, and/or improving the tumor microenvironment. For example, a CD19 CAR-expressing cell targets cancer cells that express early lineage markers, e.g., cancer stem cells and CD19-expressing cells, while the CAR-expressing cell described herein targets cancer cells that express later lineage markers, e.g., CD123. This preconditioning approach can improve the efficacy of the CAR-expressing cell described herein. In such embodiments, the CD19 CAR-expressing cell is administered prior to, concurrently with, or after administration (e.g., infusion) of a CAR-expressing cell described herein.

In embodiments, a CAR-expressing cell described herein also expresses a CAR targeting CD19, e.g., a CD19 CAR. In an embodiment, the cell expressing a CAR described herein and a CD19 CAR is administered to a subject for treatment of a cancer described herein, e.g., AML. In an embodiment, the configurations of one or both of the CAR molecules comprise a primary intracellular signaling domain and a costimulatory signaling domain. In another embodiment, the configurations of one or both of the CAR molecules comprise a primary intracellular signaling domain and two or more, e.g., 2, 3, 4, or 5 or more, costimulatory signaling domains. In such embodiments, the CAR molecule described herein and the CD19 CAR may have the same or a different primary intracellular signaling domain, the same or different costimulatory signaling domains, or the same number or a different number of costimulatory signaling domains. Alternatively, the CAR described herein and the CD19 CAR are configured as a split CAR, in which one of the CAR molecules comprises an antigen binding domain and a costimulatory domain (e.g., 4-1BB), while the other CAR molecule comprises an antigen binding domain and a primary intracellular signaling domain (e.g., CD3 zeta).

In an embodiment, the CAR described herein and the second CAR, e.g., CD19 CAR, are on the same vector or are on two different vectors. In embodiments where the CAR described herein and the second CAR, e.g., CD19 CAR, are on the same vector, the nucleic acid sequences encoding the CAR described herein and the second CAR, e.g., CD19 CAR are in the same frame, and are separated by one or more peptide cleavage sites, e.g., P2A.

In other embodiments, the CAR-expressing cell disclosed herein is administered in combination with an anti-CD19 antibody inhibitor. In one embodiment, the anti-CD19 antibody is a humanized antigen binding domain as described in WO2014/153270 (e.g., Table 3 of WO2014/153270) incorporated herein by reference, or a conjugate thereof. Other exemplary anti-CD19 antibodies or fragments or conjugates thereof, include but are not limited to, blinatumomab, SAR3419 (Sanofi), MEDI-551 (MedImmune LLC), Combotox, DT2219ARL (Masonic Cancer Center), MOR-208 (also called XmAb-5574; MorphoSys), XmAb-5871 (Xencor), MDX-1342 (Bristol-Myers Squibb), SGN-CD19A (Seattle Genetics), and AFM11 (Affimed Therapeutics). See, e.g., Hammer. MAbs. 4.5(2012): 571-77. Blinatomomab is a bispecific antibody comprised of two scFvs—one that binds to CD19 and one that binds to CD3. Blinatomomab directs T cells to attack cancer cells. See, e.g., Hammer et al.; Clinical Trial Identifier No. NCT00274742 and NCT01209286. MEDI-551 is a humanized anti-CD19 antibody with a Fc engineered to have enhanced antibody-dependent cell-mediated cytotoxicity (ADCC). See, e.g., Hammer et al.; and Clinical Trial Identifier No. NCT01957579. Combotox is a mixture of immunotoxins that bind to CD19 and CD22. The immunotoxins are made up of scFv antibody fragments fused to a deglycosylated ricin A chain. See, e.g., Hammer et al.; and Herrera et al. J. Pediatr. Hematol. Oncol. 31.12(2009):936-41; Schindler et al. Br. J. Haematol. 154.4(2011):471-6. DT2219ARL is a bispecific immunotoxin targeting CD19 and CD22, comprising two scFvs and a truncated diphtheria toxin. See, e.g., Hammer et al.; and Clinical Trial Identifier No. NCT00889408. SGN-CD19A is an antibody-drug conjugate (ADC) comprised of an anti-CD19 humanized monoclonal antibody linked to a synthetic cytotoxic cell-killing agent, monomethyl auristatin F (MMAF). See, e.g., Hammer et al.; and Clinical Trial Identifier Nos. NCT01786096 and NCT01786135. SAR3419 is an anti-CD19 antibody-drug conjugate (ADC) comprising an anti-CD19 humanized monoclonal antibody conjugated to a maytansine derivative via a cleavable linker. See, e.g., Younes et al. J. Clin. Oncol. 30.2(2012): 2776-82; Hammer et al.; Clinical Trial Identifier No. NCT00549185; and Blanc et al. Clin Cancer Res. 2011; 17:6448-58. XmAb-5871 is an Fc-engineered, humanized anti-CD19 antibody. See, e.g., Hammer et al. MDX-1342 is a human Fc-engineered anti-CD19 antibody with enhanced ADCC. See, e.g., Hammer et al. In embodiments, the antibody molecule is a bispecific anti-CD19 and anti-CD3 molecule. For instance, AFM11 is a bispecific antibody that targets CD19 and CD3. See, e.g., Hammer et al.; and Clinical Trial Identifier No. NCT02106091. In some embodiments, an anti-CD19 antibody described herein is conjugated or otherwise bound to a therapeutic agent, e.g., a chemotherapeutic agent, peptide vaccine (such as that described in Izumoto et al. 2008 J Neurosurg 108:963-971), immunosuppressive agent, or immunoablative agent, e.g., cyclosporin, azathioprine, methotrexate, mycophenolate, FK506, CAMPATH, anti-CD3 antibody, cytoxin, fludarabine, rapamycin, mycophenolic acid, steroid, FR901228, or cytokine.

Combination with a Low Dose of an mTOR Inhibitor

Methods described herein use low, immune enhancing, doses of mTOR inhibitors, e.g., allosteric mTOR inhibitors, including rapalogs such as RAD001. Administration of a low, immune enhancing, dose of an mTOR inhibitor (e.g., a dose that is insufficient to completely suppress the immune system, but sufficient to improve immune function) can optimize the performance of immune effector cells, e.g., T cells or CAR-expressing cells, in the subject. Methods for measuring mTOR inhibition, dosages, treatment regimens, and suitable pharmaceutical compositions are described in U.S. Patent Application No. 2015/01240036, hereby incorporated by reference.

Exemplary mTOR inhibitors, methods for measuring mTOR inhibition, dosages, treatment regimens, and suitable pharmaceutical compositions are also described on pages 313-320 of WO 2016/164731 filed on Apr. 8 2016, which is hereby incorporated by reference in its entirety.

mTOR inhibitors useful according to the present invention also include prodrugs, derivatives, pharmaceutically acceptable salts, or analogs thereof of any of the foregoing. mTOR inhibitors, such as RAD001, may be formulated for delivery based on well-established methods in the art based on the particular dosages described herein. In particular, U.S. Pat. No. 6,004,973 (incorporated herein by reference) provides examples of formulations useable with the mTOR inhibitors described herein.

### Methods and Biomarkers for Evaluating CAR-Effectiveness or Sample Suitability

In another aspect, the invention features a method of evaluating or monitoring the effectiveness of a CAR-expressing cell therapy (e.g., a CD123 CAR therapy), in a subject (e.g., a subject having a cancer, e.g., a hematological cancer), or the suitability of a sample (e.g., an apheresis sample) for a CAR therapy (e.g., a CD123 CAR therapy). The method includes acquiring a value of effectiveness to the CAR therapy, or sample suitability, wherein said value is indicative of the effectiveness or suitability of the CAR-expressing cell therapy. In embodiments, the method is performed as described in WO2016/057705, incorporated herein by reference.

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic.

Examples of suitable biopolymers include, but are not limited to, agar, agarose, alginate, alginate/calcium phosphate cement (CPC), beta-galactosidase (β-GAL), (1,2,3,4,6-pentaacetyl a-D-galactose), cellulose, chitin, chitosan, collagen, elastin, gelatin, hyaluronic acid collagen, hydroxyapatite, poly(3-hydroxybutyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-cancer activity, of the cells to be delivered. The biopolymer scaffold can be an injectable, e.g., a gel or a semi-solid, or a solid composition.

In some embodiments, CAR-expressing cells described herein are seeded onto the biopolymer scaffold prior to delivery to the subject. In embodiments, the biopolymer scaffold further comprises one or more additional therapeutic agents described herein (e.g., another CAR-expressing cell, an antibody, or a small molecule) or agents that enhance the activity of a CAR-expressing cell, e.g., incorporated or conjugated to the biopolymers of the scaffold. In embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology, 2015, 33:97-101; and WO2014/110591.

### Pharmaceutical Compositions and Treatments

Pharmaceutical compositions of the present invention may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages.

In some embodiments, a dose of CAR cells (e.g., CD123 CAR cells or CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., e.g., CD123 CAR cells or CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD123 CAR cells or CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD123 CAR cells or CD19 CAR cells) comprises about 1.1×10⁶-1.8×10⁷ cells/kg. In some embodiments, a dose of CAR cells (e.g., CD123 CAR cells or CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD123 CAR cells or CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e e.g., CD123 CAR cells or CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

The cells can be administered by using infusion techniques that are commonly known in immunotherapy (see, e.g., Rosenberg et al., New Eng. J. of Med. 319:1676, 1988).

In certain aspects, it may be desired to administer activated T cells to a subject and then subsequently redraw blood (or have an apheresis performed), activate T cells therefrom according to the present invention, and reinfuse the patient with these activated and expanded T cells. This process can be carried out multiple times every few weeks. In certain aspects, T cells can be activated from blood draws of from 10 cc to 400 cc. In certain aspects, T cells are activated from blood draws of 20 cc, 30 cc, 40 cc, 50 cc, 60 cc, 70 cc, 80 cc, 90 cc, or 100 cc.

The administration of the subject compositions may be carried out in any convenient manner, including by aerosol inhalation, injection, ingestion, transfusion, implantation or transplantation. The compositions described herein may be administered to a patient trans arterially, subcutaneously, intradermally, intratumorally, intranodally, intramedullary, intramuscularly, by intravenous (i.v.) injection, or intraperitoneally. In one aspect, the CAR-expressing cell (e.g., T cell or NK cell) compositions of the present invention are administered to a patient by intradermal or subcutaneous injection. In one aspect, the the CAR-expressing cell (e.g., T cell or NK cell) compositions of the present invention are administered by i.v. injection. The compositions of the CAR-expressing cell (e.g., T cell or NK cell) may be injected directly into a tumor, lymph node, or site of infection.

In a particular exemplary aspect, subjects may undergo leukapheresis, wherein leukocytes are collected, enriched, or depleted ex vivo to select and/or isolate the cells of interest, e.g., immune effector cells (e.g., T cells or NK cells). These immune effector cell (e.g., T cell or NK cell) isolates may be expanded by methods known in the art and treated such that one or more CAR constructs of the invention may be introduced, thereby creating a CAR-expressing cell (e.g., CAR T cell or CAR-expressing NK cell) of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR-expressing cell (e.g., CAR T cell or CAR-expressing NK cell) of the present invention. In an additional aspect, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment, the CAR is introduced into immune effector cells (e.g., T cells or NK cells), e.g., using in vitro transcription, and the subject (e.g., human) receives an initial administration of a CAR-expressing cell (e.g., CAR T cell or CAR-expressing NK cell) of the invention, and one or more subsequent administrations of the CAR-expressing cell (e.g., CAR T cell or CAR-expressing NK cell) of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-expressing cell (e.g., CAR T cell or CAR-expressing NK cell) of the invention are administered to the subject (e.g., human) per week, e.g., 2, 3, or 4 administrations of the CAR-expressing cell (e.g., CAR T cell or CAR-expressing NK cell) of the invention are administered per week. In one embodiment, the subject (e.g., human subject) receives more than one administration of the CAR-expressing cell (e.g., CAR T cell or CAR-expressing NK cell) per week (e.g., 2, 3 or 4 administrations per week) (also referred to herein as a cycle), followed by a week of no CAR-expressing cell (e.g., CAR T cell or CAR-expressing NK cell)administrations, and then one or more additional administration of the CAR-expressing cell (e.g., CAR T cell or CAR-expressing NK cell) (e.g., more than one administration of the CAR-expressing cell (e.g., CAR T cell or CAR-expressing NK cell)per week) is administered to the subject. In another embodiment, the subject (e.g., human subject) receives more than one cycle of CAR-expressing cell (e.g., CAR T cell or CAR-expressing NK cell), and the time between each cycle is less than 10, 9, 8, 7, 6, 5, 4, or 3 days. In one embodiment, the CAR-expressing cell (e.g., CAR T cell or CAR-expressing NK cell) are administered every other day for 3 administrations per week. In one embodiment, the CAR-expressing cell (e.g., CAR T cell or CAR-expressing NK cell) of the invention are administered for at least two, three, four, five, six, seven, eight or more weeks.

In one aspect, CAR-expressing cell (e.g., CAR T cell or CAR-expressing NK cell) (e.g., CD123 CAR-expressing cell) is generated using lentiviral viral vectors, such as lentivirus. CAR-expressing cell (e.g., CAR T cell or CAR-expressing NK cell) generated that way will have stable CAR expression.

In one aspect, CAR-expressing cells, e.g., CARTs or CAR-expressing NK cells, are generated using a viral vector such as a gammaretroviral vector, e.g., a gammaretroviral vector described herein. CAR-expressing cells, e.g., CARTs or CAR-expressing NK cells, generated using these vectors can have stable CAR expression.

In one aspect, the CAR-expressing cell (e.g., CAR T cell or CAR-expressing NK cell) transiently express CAR vectors for 4, 5, 6, 7, 8, 9, 10, 11, 12, 13, 14, 15 days after transduction. Transient expression of CARs can be effected by RNA CAR vector delivery. In one aspect, the CAR RNA is transduced into the cell (e.g., T cell or NK cell) by electroporation.

A potential issue that can arise in patients being treated using transiently expressing CAR cell (e.g., CAR T cell or CAR-expressing NK cell) (particularly with murine scFv bearing CARs) is anaphylaxis after multiple treatments.

Without being bound by this theory, it is believed that such an anaphylactic response might be caused by a patient developing humoral anti-CAR response, i.e., anti-CAR antibodies having an anti-IgE isotype. It is thought that a patient's antibody producing cells undergo a class switch from IgG isotype (that does not cause anaphylaxis) to IgE isotype when there is a ten to fourteen day break in exposure to antigen.

If a patient is at high risk of generating an anti-CAR antibody response during the course of transient CAR therapy (such as those generated by RNA transductions), CAR-expressing cell (e.g., CAR T cell or CAR-expressing NK cell)infusion breaks should not last more than ten to fourteen days.

### Cytokine Release Syndrome (CRS)

Cytokine release syndrome (CRS) is a potentially life-threatening cytokine-associated toxicity that can occur as a result of cancer immunotherapy, e.g., cancer antibody therapies or T cell immunotherapies (e.g., CAR T cells). CRS results from high-level immune activation when large numbers of lymphocytes and/or myeloid cells release inflammatory cytokines upon activation. The severity of CRS and the timing of onset of symptoms can vary depending on the magnitude of immune cell activation, the type of therapy administered, and/or the extent of tumor burden in a subject. In the case of T-cell therapy for cancer, symptom onset is typically days to weeks after administration of the T-cell therapy, e.g., when there is peak in vivo T-cell expansion. See, e.g., Lee et al. Blood. 124.2(2014): 188-95.

Symptoms of CRS can include neurologic toxicity, disseminated intravascular coagulation, cardiac dysfunction, adult respiratory distress syndrome, renal failure, and/or hepatic failure. For example, symptoms of CRS can include fever with or without rigors, fatigue, malaise, myalgias, vomiting, headache, nausea, anorexia, arthalgias, diarrhea, rash, hypoxemia, tachypnea, hypotension, widened pulse pressure, potentially diminished cardiac output (late), increased cardiac output (early), azotemia, hypofibrinogenemia with or without bleeding, elevated D-dimer, hyperbilirubinemia, transaminitis, confusion, delirium, mental status changes, hallucinations, tremor, seizures, altered gait, word finding difficulty, frank aphasia, or dymetria.

IL-6 is thought to be a mediator of CRS toxicity. See, e.g., id. High IL-6 levels may initiate a proinflammatory IL-6 signaling cascade, leading to one or more of the CRS symptoms. In some cases, the level of C-reactive protein (CRP) (a biomolecule produced by the liver, e.g., in response to IL-6) can be a measure of IL-6 activity. In some cases, CRP levels may increase several fold (e.g., several logs) during CRS. CRP levels can be measured using methods described herein, and/or standard methods available in the art.

### CRS Grading

In some embodiments, CRS can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. For example, criteria for grading CRS is provided herein as Table 20A. Unless otherwise specified, CRS as used herein refers to CRS according to the criteria of Table 20A.

TABLE 20A
CRS grading

| | |
|---|---|
| Gr1 | Supportive care only |
| Gr2 | IV therapies +/− hospitalization. |
| Gr3 | Hypotension requiring IV fluids or low-dose vasoactives or |
| | hypoxemia requiring oxygen, CPAP, or BIPAP. |
| Gr4 | Hypotension requiring high-dose vasoactives or hypoxemia |
| | requiring mechanical ventilation. |
| Gr 5 | Death |

### CRS Therapies

Therapies for CRS include IL-6 inhibitor or IL-6 receptor (IL-6R) inhibitors (e.g., tocilizumab or siltuximab), sgp130 blockers, vasoactive medications, corticosteroids, immunosuppressive agents, and mechanical ventilation. Exemplary therapies for CRS are described in International Application WO2014011984, which is hereby incorporated by reference.

Tocilizumab is a humanized, immunoglobulin Glkappa anti-human IL-6R monoclonal antibody. See, e.g., id. Tocilizumab blocks binding of IL-6 to soluble and membrane bound IL-6 receptors (IL-6Rs) and thus inhibitos classical and trans-IL-6 signaling. In embodiments, tocilizumab is administered at a dose of about 4-12 mg/kg, e.g., about 4-8 mg/kg for adults and about 8-12 mg/kg for pediatric subjects, e.g., administered over the course of 1 hour.

In some embodiments, the CRS therapeutic is an inhibitor of IL-6 signalling, e.g., an inhibitor of IL-6 or IL-6 receptor. In one embodiment, the inhibitor is an anti-IL-6 antibody, e.g., an anti-IL-6 chimeric monoclonal antibody such as siltuximab. In other embodiments, the inhibitor comprises a soluble gp130 or a fragment thereof that is capable of blocking IL-6 signalling. In some embodiments, the sgp130 or fragment thereof is fused to a heterologous domain, e.g., an Fc domain, e.g., is a gp130-Fc fusion protein such as FE301. In embodiments, the inhibitor of IL-6 signalling comprises an antibody, e.g., an antibody to the IL-6 receptor, such as sarilumab, olokizumab (CDP6038), elsilimomab, sirukumab (CNTO 136), ALD518/BMS-945429, ARGX-109, or FM101. In some embodiments, the inhibitor of IL-6 signalling comprises a small molecule such as CPSI-2364.

Exemplary vasoactive medications include but are not limited to angiotensin-11, endothelin-1, alpha adrenergic agonists, rostanoids, phosphodiesterase inhibitors, endothelin antagonists, inotropes (e.g., adrenaline, dobutamine, isoprenaline, ephedrine), vasopressors (e.g., noradrenaline, vasopressin, metaraminol, vasopressin, methylene blue), inodilators (e.g., milrinone, levosimendan), and dopamine.

Exemplary vasopressors include but are not limited to norepinephrine, dopamine, phenylephrine, epinephrine, and vasopressin. In some embodiments, a high-dose vasopressor includes one or more of the following: norpepinephrine monotherapy at ≥20 ug/min, dopamine monotherapy at ≥10 ug/kg/min, phenylephrine monotherapy at ≥200 ug/min, and/or epinephrine monotherapy at ≥10 ug/min. In some embodiments, if the subject is on vasopressin, a high-dose vasopressor includes vasopressin+norepinephrine equivalent of ≥10 ug/min, where the norepinephrine equivalent dose=[norepinephrine (ug/min)]+[dopamine (ug/kg/min)/2]+[epinephrine (ug/min)]+[phenylephrine (ug/min)/10]. In some embodiments, if the subject is on combination vasopressors (not vasopressin), a high-dose vasopressor includes norepinephrine equivalent of ≥20 ug/min, where the norepinephrine equivalent dose=[norepinephrine (ug/min)]+[dopamine (ug/kg/min)/2]+[epinephrine (ug/min)]+[phenylephrine (ug/min)/10]. See e.g., Id.

In some embodiments, a low-dose vasopressor is a vasopressor administered at a dose less than one or more of the doses listed above for high-dose vasopressors.

Exemplary corticosteroids include but are not limited to dexamethasone, hydrocortisone, and methylprednisolone. In embodiments, a dose of dexamethasone of 0.5 mg/kg is used. In embodiments, a maximum dose of dexamethasone of 10 mg/dose is used. In embodiments, a dose of methylprednisolone of 2 mg/kg/day is used.

Exemplary immunosuppressive agents include but are not limited to an inhibitor of TNFα or an inhibitor of IL-1. In embodiments, an inhibitor of TNFα comprises an anti-TNFα antibody, e.g., monoclonal antibody, e.g., infliximab. In embodiments, an inhibitor of TNFα comprises a soluble TNFα receptor (e.g., etanercept). In embodiments, an IL-1 or IL-1R inhibitor comprises anakinra.

In some embodiments, the subject at risk of developing severe CRS is administered an anti-IFN-gamma or anti-sIL2Ra therapy, e.g., an antibody molecule directed against IFN-gamma or sIL2Ra.

In embodiments, for a subject who has received a therapeutic antibody molecule such as blinatumomab and who has CRS or is at risk of developing CRS, the therapeutic antibody molecule is administered at a lower dose and/or a lower frequency, or administration of the therapeutic antibody molecule is halted.

In embodiments, a subject who has CRS or is at risk of developing CRS is treated with a fever reducing medication such as acetaminophen.

In embodiments, a subject herein is administered or provided one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject at risk of developing CRS (e.g., severe CRS) (e.g., identified as having a high risk status for developing severe CRS) is administered one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation, in any combination, e.g., in combination with a CAR-expressing cell described herein.

In embodiments, a subject herein (e.g., a subject at risk of developing severe CRS or a subject identified as at risk of developing severe CRS) is transferred to an intensive care unit. In some embodiments, a subject herein (e.g., a subject at risk of developing severe CRS or a subject identified as at risk of developing severe CRS) is monitored for one ore more symptoms or conditions associated with CRS, such as fever, elevated heart rate, coagulopathy, MODS (multiple organ dysfunction syndrome), cardiovascular dysfunction, distributive shock, cardiomyopathy, hepatic dysfunction, renal dysfunction, encephalopathy, clinical seizures, respiratory failure, or tachycardia. In some embodiments, the methods herein comprise administering a therapy for one of the symptoms or conditions associated with CRS. For instance, in embodiments, e.g., if the subject develops coagulopathy, the method comprises administering cryoprecipitate. In some embodiments, e.g., if the subject develops cardiovascular dysfunction, the method comprises administering vasoactive infusion support. In some embodiments, e.g., if the subject develops distributive shock, the method comprises administering alpha-agonist therapy. In some embodiments, e.g., if the subject develops cardiomyopathy, the method comprises administering milrinone therapy. In some embodiments, e.g., if the subject develops respiratory failure, the method comprises performing mechanical ventilation (e.g., invasive mechanical ventilation or noninvasive mechanical ventilation). In some embodiments, e.g., if the subject develops shock, the method comprises administering crystalloid and/or colloid fluids.

In embodiments, the CAR-expressing cell is administered prior to, concurrently with, or subsequent to administration of one or more therapies for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation. In embodiments, the CAR-expressing cell is administered within 2 weeks (e.g., within 2 or 1 week, or within 14 days, e.g., within 14, 13, 12, 11, 10, 9, 8, 7, 6, 5, 4, 3, 2, 1 day or less) of administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation. In embodiments, the CAR-expressing cell is administered at least 1 day (e.g., at least 1, 2, 3, 4, 5, 6, 7, 8, 9, 10, 11, 12, 13, 14, 15, 16, 17, 18, 19, 20, 1, week, 2 weeks, 3 weeks, 4 weeks, 1 month, 2 months, 3 months, 3 months, or more) before or after administration of one or more therapies for CRS described herein, e.g., one or more of IL-6 inhibitors or IL-6 receptor (IL-6R) inhibitors (e.g., tocilizumab), vasoactive medications, corticosteroids, immunosuppressive agents, or mechanical ventilation.

In embodiments, a subject herein (e.g., a subject at risk of developing severe CRS or a subject identified as at risk of developing severe CRS) is administered a single dose of an IL-6 inhibitor or IL-6 receptor (IL-6R) inhibitor (e.g., tocilizumab). In embodiments, the subject is administered a plurality of doses (e.g., 2, 3, 4, 5, 6, or more doses) of an IL-6 inhibitor or IL-6 receptor (IL-6R) inhibitor (e.g., tocilizumab).

In embodiments, a subject at low or no risk of developing CRS (e.g., severe CRS) (e.g., identified as having a low risk status for developing severe CRS) is not administered a therapy for CRS described herein, e.g., one or more of IL-6 inhibitor or IL-6 receptor (IL-6R) inhibitors (e.g., tocilizumab), vasoactive medications, corticosteroids, immunosuppressive agents, or mechanical ventilation.

In embodiments, a subject is determined to be at high risk of developing severe CRS by using an evaluation or prediction method described herein. In embodiments, a subject is determined to be at low risk of developing severe CRS by using an evaluation or prediction method described herein.

### Identifying a Subject at Risk for CRS

### Use of Biomarkers to Evaluate (e.g., Predict) CRS Severity

In embodiments, one or more biomarkers are used to evaluate (e.g., predict) CRS severity. Exemplary biomarkers used to evaluate (e.g., predict) CRS severity include cytokines such as sTNFR2, IP10, sIL1R2, sTNFR1, M1G, VEGF, sILR1, TNFα, IFNα, GCSF, sRAGE, IL4, IL10, IL1R1, IFN-γ, IL6, IL8, sIL2Rα, sgp130, sIL6R, MCP1, MIP1α, MIP1β, and GM-CSF. In embodiments, one or more (e.g., two or more, or three or more) of the cytokines, sTNFR2, IP10, sIL1R2, sTNFR1, M1G, VEGF, sILR1, TNFα, IFNα, GCSF, sRAGE, IL4, IL10, IL1R1, IFN-γ, IL6, IL8, sIL2Rα, sgp130, sIL6R, MCP1, MIP1α, MIP1β, and GM-CSF, are used to evaluate (e.g., predict) CRS severity. In embodiments, one or more (e.g., two or more, or three or more) of the cytokines, IFN-γ, IL6, IL8, sIL2Rα, sgp130, sIL6R, MCP1, MIP1α, MIP1β, and GM-CSF, are used to evaluate (e.g., predict) CRS severity. In embodiments, one or more (e.g., both) of the cytokines, IFN-γ and sgp130, are used to evaluate (e.g., predict) CRS severity, e.g., in an adult or pediatric subject. In embodiments, one or more (e.g., two or more, or all three) of the cytokines, IFN-γ, sgp130, and IL1Ra, are used to evaluate (e.g., predict) CRS severity, e.g., in an adult or pediatric subject. In embodiments, one or more (e.g., two or more, or all three) of the cytokines, IFN-γ, IL13, and MIP1α are used to evaluate (e.g., predict) CRS severity, e.g., in a pediatric subject. In embodiments, one or more (e.g., two or more, or all three) of the cytokines, sgp130, MCP1, and eotaxin are used to evaluate (e.g., predict) CRS severity, e.g., in a pediatric or adult subject. In embodiments, one or more (e.g., two or more, or all three) of the cytokines, IL2, eotaxin, and sgp130 are used to evaluate (e.g., predict) CRS severity, e.g., in a pediatric or adult subject. In embodiments, one or more (e.g., two or more, or all three) of the cytokines, IFN-gamma, IL2, and eotaxin are used to evaluate (e.g., predict) CRS severity, e.g., in a pediatric subject. In embodiments, one or more (e.g., both) of IL10 and disease burden are used to evaluate (e.g., predict) CRS severity, e.g., in a pediatric subject. In embodiments, one or more (e.g., both) of the cytokines, IFN-gamma and IL-13 eotaxin are used to evaluate (e.g., predict) CRS severity, e.g., in a pediatric subject. In embodiments, one or more (e.g., two or more, or all three) of the cytokines, IFN-gamma, IL-13, and MIP1-alpha, are used to evaluate (e.g., predict) CRS severity, e.g., in a pediatric subject. In embodiments, one or more (e.g., both) of the cytokines IFN-gamma and MIP1-alpha, are used to evaluate (e.g., predict) CRS severity, e.g., in a pediatric subject.

Exemplary biomarkers used to evaluate (e.g., predict) CRS severity can also include disease burden assessments, e.g., the extent of disease (e.g., cancer) in a subject. For example, a disease burden assessment can be made by determining the level of disease (e.g., cancer) in a biological sample from a subject (e.g., bone marrow of a subject). For example, a high disease burden is indicated by the presence of at least 25% (e.g., at least 25%, 30%, 35%, 40%, 45%, 50%, 55%, 60%, 70%, 80%, 90% or higher) bone marrow blasts (e.g., determined by morphology on an aspirate or biopsy, a flow assay on an aspirate or biopsy, and/or by MRD). In some embodiments, a high disease burden is indicated by the presence of at least 50% bone marrow blasts. For example, a low disease burden is indicated by the presence of less than 25% (e.g., 24% or less, e.g., 24%, 23%, 22%, 21%, 20%, 15%, 10%, 5% or less) bone marrow blasts (e.g., determined by morphology on an aspirate or biopsy, a flow assay on an aspirate or biopsy, and/or by MRD). In some embodiments, a low disease burden is indicated by the presence of less than 0.1%, 1%, 5%, 25%, or 50% bone marrow blasts. In some embodiments, the cancer is ALL. In embodiments, the cancer is AML.

In embodiments, one or more cytokines in combination with a disease burden assessment is used to evaluate (e.g., predict) CRS severity, e.g., in a pediatric subject. In embodiments, one or more of the cytokines, spg130 and IFN-γ, in combination with bone marrow disease (e.g., cancer) are used to evaluate (e.g., predict) CRS severity, e.g., in a pediatric subject. In embodiments, disease burden assessments, e.g., from bone marrow, e.g., for cancer, can be determined used methods described herein, e.g., as described in Borowitz et al. Blood. 2008; 111(12):5477-85; or Weir et al. Leukemia. 1999; 13(4):558-67.

Another exemplary biomarker used to evaluate (e.g., predict) CRS severity includes C-reactive protein (CRP) level or activity. In embodiments, a subject at low risk of severe CRS is identified as having a CRP level of less than 7 mg/dL (e.g., 7, 6.8, 6, 5, 4, 3, 2, 1 mg/dL or less). In embodiments, a subject at high risk of severe CRS is identified as having a greater level of CRP in a sample (e.g., a blood sample) compared to a subject at low risk of severe CRS or compared to a control level or activity. In embodiments, the greater level or activity is at least 2-fold greater (e.g., at least 2, 3, 4, 5, 6, 7, 8, 9, 10, 15, 20, 25, 30, 40, 50, 100, 500, 1000-fold or more greater) compared to a subject at low risk of severe CRS or compared to a control level or activity.

In embodiments, the biomarkers described herein are used to predict CRS severity in a subject early on after administration with a CAR T cell (e.g., a CAR T cell described herein, e.g., a CD19 CAR-expressing cell therapy described herein such as, e.g., CTL019; or a CD123 CAR-expressing cell). In embodiments, the biomarkers described herein are used to predict CRS severity in a subject within 2 weeks, e.g., within 1 week or less after administration with the CAR T cell. In embodiments, the biomarkers described herein are used to predict CRS severity in a subject within 10 days (e.g., 10, 9, 8, 7, 6, 5, 4, 3, 2, 1 day or less after administration with the CAR T cell. In embodiments, the biomarkers described herein are used to predict CRS severity in a subject within 1-10 days (e.g., within 1-10, 1-9, 1-8, 1-7, 1-6, 1-5, 1-4, 1-3, 1-2, or 1 day after administration with the CAR T cell. In embodiments, the biomarkers described herein are used to predict CRS severity in a subject before the subject experiences one or more symptoms of grade 2, 3, 4, or 5 CRS (e.g., before the subject experiences one or more symptoms of grade 3, 4, or 5 CRS, or grade 4 or 5 CRS).

In embodiments, one or more (e.g., both) of the cytokines, IFN-γ and sgp130, are used to predict CRS severity, e.g., in an adult or pediatric subject, within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019; or a CD123 CAR-expressing cell).

In embodiments, one or more (e.g., two or more, or all three) of the cytokines, IFN-γ, sgp130, and IL1Ra, are used to predict CRS severity, e.g., in an adult or pediatric subject, within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019; or a CD123 CAR-expressing cell).

In embodiments, one or more (e.g., two or more, or all three) of the cytokines, IFN-γ, IL13, and MIP1a are used to predict CRS severity, e.g., in a pediatric subject, within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019; or a CD123 CAR-expressing cell).

In embodiments, one or more of the cytokines, spg130 and IFN-γ, in combination with bone marrow disease (e.g., cancer) are used to predict CRS severity, e.g., in a pediatric subject, within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019; or a CD123 CAR-expressing cell).

In embodiments, CRP level or activity is used to predict CRS severity, e.g., in an adult or pediatric subject, within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019; or a CD123 CAR-expressing cell).

In embodiments, elevated or reduced levels of one or more of the cytokines described herein, e.g., sTNFR2, IP10, sIL1R2, sTNFR1, M1G, VEGF, sILR1, TNFα, IFNα, GCSF, sRAGE, IL4, IL10, IL1R1, IFN-γ, IL6, IL8, sIL2Rα, sgp130, sIL6R, MCP1, MIP1α, MIP1β, and GM-CSF, relative to a control level, indicate that the subject is at high risk of developing severe CRS.

In embodiments, levels of one or more of the cytokines described herein, e.g., sTNFR2, IP10, sIL1R2, sTNFR1, M1G, VEGF, sILR1, TNFα, IFNα, GCSF, sRAGE, IL4, IL10, IL1R1, IFN-γ, IL6, IL8, sIL2Rα, sgp130, sIL6R, MCP1, MIP1α, MIP1β, and GM-CSF, that are elevated or lowered relative to a reference level, indicate that the subject is at high risk of developing severe CRS. In embodiments, levels of one or more of the cytokines described herein that are elevated by at least 2-fold (e.g., 2, 3, 4, 5, 6, 7, 8, 9, 10, 50, 100, 500, 1000-fold or more) relative to a control level (e.g., a baseline level), indicate that the subject is at high risk of developing severe CRS. In embodiments, levels of one or more of the cytokines described herein that are lowered by at least 10% (e.g., at least 20%, 30%, 40%, 50%, 60%, 70%, 80%, 90%, 95%, or 99%) relative to a reference level, indicate that the subject is at high risk of developing severe CRS. In some embodiments, the reference level is a value that does not depend on the baseline level of the cytokine in the subject. In some embodiments, the reference level is baseline cytokine value or baseline cytokine values by disease burden.

In embodiments, levels of one or more of the cytokines described herein, e.g., sTNFR2, IP10, sIL1R2, sTNFR1, M1G, VEGF, sILR1, TNFα, IFNα, GCSF, sRAGE, IL4, IL10, IL1R1, IFN-γ, IL6, IL8, sIL2Rα, sgp130, sIL6R, MCP1, MIP1α, MIP1β, and GM-CSF, that are elevated or lowered relative to a reference level, indicate that the subject is at high risk of developing severe CRS.

In embodiments, levels of one or more (e.g., both) of the cytokines, IFN-γ and sgp130, that are elevated, e.g., by at least 2-fold (e.g., 2, 3, 4, 5, 6, 7, 8, 9, 10, 50, 100, 500, 1000-fold or more) relative to a control level, e.g., when measured within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019), indicate that the subject is at high risk of developing severe CRS, e.g., where the subject is an adult or pediatric subject. In embodiments, the control level is a level of IFN-γ and/or sgp130 of a normal, healthy adult or pediatric subject (e.g., without CRS); or of the subject prior to administration of a CAR-expressing cell.

In embodiments, levels of one or more (e.g., two or more, or all three) of the cytokines, IFN-γ, sgp130, and IL1Ra, that are altered, e.g., by at least 2-fold (e.g., 2, 3, 4, 5, 6, 7, 8, 9, 10, 50, 100, 500, 1000-fold or more) relative to a control level, e.g., when measured within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019), indicate that the subject is at high risk of developing severe CRS, e.g., where the subject is an adult or pediatric subject. In embodiments, the altered level is a greater level of sgp130, a greater level of IFN-gamma, or a lower level of IL1Ra, or any combination thereof. In embodiments, the control level is a level of IFN-γ and/or sgp130 of a normal, healthy adult or pediatric subject (e.g., without CRS); or of the subject prior to administration of a CAR-expressing cell.

In embodiments, levels of one or more (e.g., two or more, or all three) of the cytokines, IFN-γ, IL13, and MIP1α, that are altered, e.g., by at least 2-fold (e.g., 2, 3, 4, 5, 6, 7, 8, 9, 10, 50, 100, 500, 1000-fold or more) relative to a control level, e.g., when measured within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019), indicate that the subject is at high risk of developing severe CRS, e.g., where the subject is a pediatric subject. In embodiments, the altered level is a greater level of IFN-gamma, a lower level of IL-13, a lower level of MIP1-alpha, or any combination thereof. In embodiments, the control level is a level of IFN-γ and/or sgp130 of a normal, healthy pediatric subject (e.g., without CRS); or of the subject prior to administration of a CAR-expressing cell.

In embodiments, a combination of altered levels of one or more of the cytokines, spg130 and IFN-γ, relative to a control level, and a high disease burden (e.g., bone marrow disease), e.g., when measured within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019), indicate that the subject is at high risk of developing severe CRS, e.g., where the subject is a pediatric subject. In embodiments, the altered level is a greater level of spg130, a greater level of IFN-gamma, and a greater level of disease burden. In embodiments, the control level is a level of IFN-γ and/or sgp130 of a normal, healthy pediatric subject (e.g., without CRS); or of the subject prior to administration of a CAR-expressing cell.

In embodiments, a CRP level of less than 7 mg/dL (e.g., 7, 6.8, 6, 5, 4, 3, 2, 1 mg/dL or less), e.g., when measured within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019; or a CD123 CAR-expressing cell), indicate that the subject is at low risk of developing severe CRS.

In embodiments, a CRP level of 6 mg/dL or greater (e.g., 6, 6.8, 7, 8, 9, 10, 11, 12, 13, 14, 15, 16, 17, 18, 19, 20, 22, 24, 26, 28, 30, 32, 34, 36, 38, 40 mg/dL or greater), e.g., when measured within 1-10 days (e.g., within 1-10, 1-9, 1-8, 1-7, 1-6, 1-5, 1-4, 1-3, 1-2, or 1 day after administration with a CAR T cell (e.g., a CAR T cell described herein, e.g., a CD19 CAR-expressing cell therapy described herein such as, e.g., CTL019; or a CD123 CAR-expressing cell), indicate that the subject is at high risk of developing severe CRS.

In certain aspects, the disclosure provides a method of monitoring CRS (e.g., monitoring a patient having CRS0, CRS1, CSR2, or CRS3) or monitoring for the development of severe CRS, comprising evaluating one or more CRS biomarkers herein. The method can involve measuring the one or more biomarkers at a plurality of timepoints, e.g., at 2, 3, 4, 5, 6, 7, 8, 9, 10, or more timepoints. In certain aspects, the disclosure provides a method of managing CRS, comprising evaluating a subject at risk for developing CRS (e.g., severe CRS), and optionally administering a treatment for CRS, e.g., a treatment described herein.

Certain cytokines can be referred to by one or more synonynms. For example, IL1R1 and IL1RA, as used herein, are both synonyms for the IL1 receptor. sIL_1RI is a synonym for sILR1. sIL_1RII is a synonym for sIL1R2.

In embodiments, a subject is identified as at risk for CRS if the subject has a high tumor burden, e.g., prior to administration of a CAR therapy (e.g., a CAR therapy described herein), e.g., as described in Maude & Frey et al, NEJM 2014.

### Identifying a Subject Having CRS

### Use of Laboratory Tests to Determine Whether a Subject has Severe CRS

In some aspects, the invention features a method of determining whether a subject has severe CRS. The method includes acquiring a CRS risk status, e.g., in response to an immune cell based therapy, e.g., a CAR-expressing cell therapy (e.g., a CAR19-expressing cell therapy or a CAR123-expressing cell therapy) for the subject, wherein said CRS risk status includes a measure of one, two, or more (all) of the following:

(i) the level or activity of one or more (e.g., 3, 4, 5, 10, 15, 20, or more) cytokines chosen from sTNFR2, IP10, sIL1R2, sTNFR1, M1G, VEGF, sILR1, TNFα, IFNα, GCSF, sRAGE, IL4, IL10, IL1R1, IFN-γ, IL6, IL8, sIL2Rα, sgp130, sIL6R, MCP1, MIP1α, MIP1β, or GM-CSF, or laboratory tests (e.g., analytes) chosen from C-reactive protein (CRP), ferritin, lactate dehydrogenase (LDH), aspartate aminotransferase (AST), or blood urea nitrogen (BUN), alanine aminotransferase (ALT), creatinine (Cr), or fibrinogen, Prothrombin Time (PT), Partial Thromboplastin Time (PTT), or a combination thereof, in a sample (e.g., a blood sample);

(ii) the level or activity of IL6, IL6R, or sgp130, or a combination thereof (e.g., a combination of any two or all three of IL6, IL6R, and sgp130), in a sample (e.g., a blood sample); or

(iii) the level or activity of IL6, IFN-gamma, or IL2R, or a combination thereof (e.g., a combination of any two or all three of IL6, IFN-gamma, and IL2R), in a sample (e.g., a blood sample);

wherein the value is indicative of the subject's severe CRS status.

In some embodiments, a ferritin level of at least about 23,500, 25,000, 30,000, 40,000, 50,000, 70,000, 80,000, 90,000, 100,000, 150,000, 200,000, or 250,000 ng/ml, and optionally up to about 299,000 or 412,000 ng/ml, is indicative of severe CRS. In some embodiments, a ferritin level of less than about 23,500, 20,000, 18,000, 16,000, 14,000, 12,000, 10,000, 9,000, 8,000, 7,000, 6,000 5,000, 4,000, 3,000, 2,000, or 1,000 ng/ml and optionally greater than about 280 ng/ml, is indicative that the subject does not have severe CRS.

In some embodiments, a LDH level of at least about 1,700, 2,000, 3,000, 4,000, 5,000, 6,000, 7,000, 8,000, 9,000, 10,000, 15,000, or 20,000 U/L, and optionally up to about 24,000 U/L, is indicative of severe CRS. In some embodiments, a LDH level of less than about 1,700, 1,500, 1,400, 1,300, 1,200, 1,100, 1,000, 900, 800, 700, 600, 500, 400, 300, or 200 U/L, and optionally greater than about 159 U/L, is indicative that the subject does not have severe CRS.

In some embodiments, a CRP level of at least about 20, 21, 22, 23, 24, 25, 26, 27, 28, 29, 30, 31, 32, 33, 34, or 35 mg/dl, and optionally up to about 38 mg/dl, is indicative of severe CRS. In some embodiments, a CRP level of less than about 20, 19, 18, 17, 16, 15, 14, 13, 12, 11, 10, 9, 8, 7, 6, 5, 4, 3, 2, or 1 mg/dl, and optionally greater than about 0.7 mg/dl, is indicative that the subject does not have severe CRS.

In some embodiments, an ALT level of at least about 100, 110, 120, 130, 140, 150, 160, 170, 180, 190, 200, 250, 300, 350, 400, 450, 500, 550, 600, 650, 700, 750, 800, 980, 900, 950, or 1000 U/L, and optionally up to 1300 U/L, is indicative of severe CRS. In some embodiments, an ALT level of less than about 100, 90, 80, 70, 60, 50, 40, or 30 U/L, and optionally greater than about 25 U/L, is indicative that the subject does not have severe CRS.

In some embodiments, an AST level of at least about 150, 200, 250, 300, 350, 400, 450, 500, 550, 600, 650, 700, 750, 800, 980, 900, 950, 1000 U/L, and optionally up to about 1500 U/L, is indicative of severe CRS. In some embodiments, an AST level of less than about 150, 140, 130, 120, 100, 90, 80, 70, 60, 50, 40, or 30 U/L, and optionally greater than about about 15 U/L, is indicative that the subject does not have severe CRS.

In some embodiments, a BUN level of at least about 18, 19, 20, 25, 30, 35, 40, 45, 50, 60, 70, 80, 90, 100, 110, 120, 130, 140, 150, 160, 170, 180, or 190 mg/dl, and optionally up to about 210 mg/dl, is indicative of severe CRS. In some embodiments, a BUN level of less than about 18, 17, 16, 15, 14, 13, 12, 11, or 10 mg/dl, and optionally greater than about 5 mg/dl, is indicative that the subject does not have severe CRS.

In some embodiments, a fibrinogen level of less than about 150, 140, 130, 120, 110, 100, 90, 80, 70, 60, 50, 40, or 30 mg/dl, and optionally greater than about 20 mg/dl, is indicative of severe CRS. In some embodiments, a fibrinogen level of at least about 150, 160, 170, 180, 190, 200, or 210 mg/dl, and optionally up to about 230 mg/dl, is indicative that the subject does not have severe CRS.

In some embodiments, a PT level of at least about 17, 18, 19, 20, 21, or 22 sec, and optionally up to about 24 sec, is indicative of severe CRS. In some embodiments, a PT level of less than about 17, 16, 15, or 14 sec, and optionally greater than about 12 sec, is indicative that the subject does not have severe CRS.

In some embodiments, a PTT level of at least about 44, 45, 46, 47, 48, 49, 50, 51, 52, 53, 54, 55, 60, 65, 70, 75, 80, or 85 sec, and optionally up to about 95 sec, indicative of severe CRS. In some embodiments, a PTT level of less than about 44, 43, 42, 41, 40, 39, 38, 37, 36, 35, 34, 33, 32, 31, 30, 29, 28, or 27 sec, and optionally greater than about 25 sec, is indicative that the subject does not have severe CRS.

In some embodiments, a patient with severe CRS has an IFN-□>75 pg/ml and IL-10 >60 pg/ml. In some embodiments, a patient with severe CRS has an IFN-□ of greater than or equal to 40, 50, 60, 70, or 75 pg/ml, an IL-10 level of greater than or equal to 30, 40, 50, or 60 pg/ml, or any combination thereof.

### Biomarkers Assessment

In accordance with any method described herein, e.g., involving identifying a subject at risk for developing CRS or identifying a subject having CRS, one or more biomarkers can be assessed, e.g., using a method described herein.

In some embodiments, the amount of the biomarker determined in a sample from a subject is quantified as an absolute measurement (e.g., ng/mL). Absolute measurements can easily be compared to a reference value or cut-off value. For example, a cut-off value can be determined that represents a disease progressing status; any absolute values falling either above (i.e., for biomarkers that increase expression with progression of a cancer, e.g., a hematological cancer such as ALL and CLL) or falling below (i.e., for biomarkers with decreased expression with progression of a cancer, e.g., a hematological cancer such as ALL and CLL) the cut-off value are likely to be disease progressing.

Alternatively, the relative amount of a biomarker is determined. In one embodiment, the relative amount is determined by comparing the expression and/or activity of one or more biomarkers in a subject with cancer to the expression of the biomarkers in a reference parameter. In some embodiments, a reference parameter is obtained from one or more of: a baseline or prior value for the subject, the subject at a different time interval, an average or median value for a cancer subject (e.g., patient) population, a healthy control, or a healthy subject population.

The present disclosure also pertains to the field of predictive medicine in which diagnostic assays, pharmacogenomics, and monitoring clinical trials are used for predictive purposes to thereby treat an individual prophylactically. Accordingly, one aspect of the present disclosure relates to assays for determining the amount, structure, and/or activity of polypeptides or nucleic acids corresponding to one or more markers described herein, in order to determine whether an individual having cancer (e.g., a hematological cancer such as CLL and ALL) or at risk of developing cancer (e.g., a hematological cancer such as CLL and ALL) will be more likely to respond to CAR-expressing cell therapy (e.g., a CD19 CAR-expressing cell therapy described herein such as, e.g., CTL019; or a CAR123-expressing cell therapy).

### Methods for Detection of Gene Expression

Biomarker expression level can also be assayed. Expression of a marker described herein can be assessed by any of a wide variety of known methods for detecting expression of a transcribed molecule or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In certain embodiments, activity of a particular gene is characterized by a measure of gene transcript (e.g., mRNA), by a measure of the quantity of translated protein, or by a measure of gene product activity. Marker expression can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

Methods of detecting and/or quantifying the gene transcript (mRNA or cDNA made therefrom) using nucleic acid hybridization techniques are known to those of skill in the art (see e.g., Sambrook et al. supra). For example, one method for evaluating the presence, absence, or quantity of cDNA involves a Southern transfer as described above. Briefly, the mRNA is isolated (e.g., using an acid guanidinium-phenol-chloroform extraction method, Sambrook et al. supra.) and reverse transcribed to produce cDNA. The cDNA is then optionally digested and run on a gel in buffer and transferred to membranes. Hybridization is then carried out using the nucleic acid probes specific for the target cDNA.

Methods to measure biomarkers described herein, include, but are not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF) mass spectrometry, microcytometry, microarray, microscopy, fluorescence activated cell sorting (FACS), flow cytometry, laser scanning cytometry, hematology analyzer and assays based on a property of the protein including but not limited to DNA binding, ligand binding, or interaction with other protein partners.

A kit of the invention can comprise a reagent useful for determining protein level or protein activity of a marker.

### Subjects

For any of the methods and kits disclosed herein, the subject treated, or the subject evaluated, is a subject having, or at risk of having, cancer at any stage of treatment. Cancers are described in greater detail above. For example, cancers include, but are not limited to, B-cell acute lymphocytic leukemia (B-ALL), T-cell acute lymphocytic leukemia (T-ALL), acute lymphocytic leukemia (ALL), chronic myelogenous leukemia (CML), chronic lymphocytic leukemia (CLL), B cell promyelocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma (MCL), marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, and Waldenstrom macroglobulinemia. In an embodiment, the cancer is a hematological cancer. In a preferred embodiment, the cancer is AML. In a preferred embodiment, the cancer is ALL. In another preferred embodiment, the cancer is CLL. In an embodiment, the cancer is associated with CD19 expression. In embodiments, the cancer is associated with CD123 expression.

In other embodiments, for any of the methods and kits disclosed herein, the subject treated, or the subject evaluated, is a subject to be treated or who has been treated with a CAR T cell, e.g., a CD19 CAR-expressing cell, e.g., CTL-019; or a CD123 CAR-expressing cell.

In embodiments, the subject is an adult subject, e.g., having an age of greater than 18 years (e.g., 18, 19, 20, 21, 22, 23, 24, 25, 26, 27, 28, 29, 30, years of age or older, 30-35, 35-40, 40-45, 45-50, 50-55, 55-60, 60-65, 65-70, 70-75, 75-80, 80-85, 85-90, 90-95, or 95-100 years of age).

In embodiments, the subject is a pediatric subject, e.g., having an age less than 18 (e.g., 17, 16, 15, 14, 13, 12, 11, 10, 9, 8, 7, 6, 5, 4, 3, 2, or 1 year of age or younger).

In embodiments, the subject is at risk (e.g., at high risk) for developing CRS (e.g., severe CRS). In embodiments, the subject is at low risk (e.g., not at risk) for developing CRS (e.g., severe CRS).

In embodiments, the subject has CRS0, CRS1, CRS2, or CRS3.

In embodiments, the risk of a subject for developing CRS (e.g., severe CRS) is determined using an evaluation or prediction method described herein.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples specifically point out various aspects of the present invention, and are not to be construed as limiting in any way the remainder of the disclosure.

### Example 1: Ruxolitinib Treatment Prevented Cytokine Release Syndrome after Chimeric Antigen Receptor T Cell Therapy

Chimeric antigen receptor T (CART) cell therapy results in impressively high remission rates in B cell acute lymphoid leukemia (ALL), but can in some cases can result in development of cytokine release syndrome (CRS). See, e.g., Porter et al. Sci Transl Med. 2015; 7:303ra139; Maude et al. N Engl J Med. 2014; 371:1507-1517; Lee et al. Lancet. 2015; 385:517-528; Davila et al. Sci Transl Med. 2014; 6:224ra225; Kochenderfer et al. J Clin Oncol. 2014; Kalos et al. Sci Transl Med. 2011; 3:95ra73; Porter et al. N Engl J Med. 2011; 365:725-733; and Grupp et al. N Engl J Med. 2013; 368:1509-1518. CRS is characterized by the development of high-grade fevers, hypotension, fluid overload and respiratory compromise, coincides with T cell expansion and is associated with marked elevation of interleukin-6, interferon-γ and other inflammatory cytokines. Severe CRS is seen in 25-80% of patients treated with CD19 directed CART cell therapy (CART19) and mortality has been reported. As such, there is a need for CRS treatment and prevention. See, e.g., Porter et al. Sci Transl Med. 2015; 7:303ra139; Maude et al. N Engl J Med. 2014; 371:1507-1517; Lee et al. Lancet. 2015; 385:517-528; and Davila et al. Sci Transl Med. 2014; 6:224ra225.

While the use of the anti-IL6 receptor antibody tocilizumab with or without steroids can sometimes reverse CRS, there is concern that the early introduction of immunosuppressive medications could impair the anti-tumor activity. See, e.g., Grupp et al. N Engl J Med. 2013; 368:1509-1518. Therefore, most investigators currently reserve tocilizumab as therapy for severe (grade 3-4) CRS. See, e.g., Lee et al. Blood. 2014; 124:188-195. The presence of high tumor burden can be a predictor of severe CRS, and cytoreductive chemotherapy could potentially reduce the incidence of severe CRS. See, e.g., Maude et al. N Engl J Med. 2014; 371:1507-1517. However, most patients undergoing CART19 therapy are chemorefractory, and as such, cytoreduction may not be possible. Predictive models based on early post-treatment cytokine elevation have been developed but rely on the timely availability of these results. See, e.g., Teachey et al. Blood. 2015; 126:1334-1334. Therefore, a well-tolerated, clinically available pharmacologic intervention that does not abrogate the anti-tumor effect would represent a vertical advance in the field.

There is a lack of models, e.g., preclinical models, for CRS after human CART therapy. For example, CART19 therapy of ALL xenografts does not induce CRS. The lack of models has limited the development of CRS prevention modalities. A way to prevent CRS would greatly enhance the feasibility of CART therapy. (See, e.g., van der Stegen S J, Davies D M, Wilkie S, et al. Preclinical in vivo modeling of cytokine release syndrome induced by ErbB-retargeted human T cells: identifying a window of therapeutic opportunity? J Immunol. 2013; 191:4589-4598).

This Example describes the generation/characterization of a xenograft acute myeloid leukemia model (a preclinical AML xenograft model of CRS), which can be used to study CRS after CART cell therapy. The results herein show that the JAK/STAT inhibitor ruxolitinib could prevent CRS. Ruxolitinib blunted the in vivo T cell proliferation and cytokine production that is associated with severe CRS, without impairing the anti-tumor effect of CART cells. These results may support the incorporation of JAK inhibitors such as ruxolitinib into future clinical trials in combination with CART cell therapy in patients with high risk for the development of severe CRS.

### MATERIALS and METHODS

### Cell Lines and Primary Samples.

Cell lines were originally obtained from ATCC. For some experiments, MOLM14 cell line was transduced with firefly luciferase/eGFP and then sorted to obtain a >99% positive population. The cell lines were maintained in culture with RPMI media supplemented with 10% fetal bovine serum and 50 IU/ml penicillin/streptomycin. De-identified primary human AML specimens were obtained from the University of Pennsylvania stem cell and xenograft core. For all functional studies, primary cells were thawed and rested at 37° C. for at least 12 hours.

### Generation of CAR Constructs and CAR T Cells.

CD123 directed CAR constructs and CART cells were generated as previously described. See, e.g., Gill S, Tasian S K, Ruella M, et al. Preclinical targeting of human acute myeloid leukemia and myeloablation using chimeric antigen receptor-modified T cells. Blood. 2014; 123:2343-2354; and Kenderian S S, Ruella M, Shestova 0, et al. CD33 Specific Chimeric Antigen Receptor T Cells Exhibit Potent Preclinical Activity against Human Acute Myeloid Leukemia. Leukemia. 2015.

### In Vitro T-Cell Effector Function Assays.

T cell degranulation, cytokine, proliferation, cytotoxicity measurements were performed as previously described. See, e.g., Kenderian S S, Ruella M, Shestova 0, et al. CD33 Specific Chimeric Antigen Receptor T Cells Exhibit Potent Preclinical Activity against Human Acute Myeloid Leukemia. Leukemia. 2015.

Animal experiments.

For the development of CRS preclinical models, NOD-SCID-γ chain−/− (NSG) transgenic for human interleukin-3, stem cell factor and granulocyte macrophage colony-stimulating factor (NSG-S) were used. These were purchased from the Stem Cell and Xenograft Core of the University of Pennsylvania (originally obtained from Jackson Laboratories). Schemas of the utilized xenograft models are discussed in detail in the relevant figures and the Results section herein. Cells were injected in 200 ul of phosphate-buffered saline at the indicated concentration into the tail veins.

### Ruxolitinib.

Ruxolitinib was purchased from Selleckchem, dissolved in DMSO and diluted to the indicated concentrations. For animal experiments, ruxolitinib was further diluted in 10% HP-beta-cyclodextrin solution (1.6 mg/ml) and was administered to mice by oral gavage at the indicated concentrations.^{12,15,16} See, e.g., Quintas-Cardama A, Vaddi K, Liu P, et al. Preclinical characterization of the selective JAK1/2 inhibitor INCB018424: therapeutic implications for the treatment of myeloproliferative neoplasms. Blood. 2010; 115:3109-3117; Das R, Guan P, Sprague L, et al. Janus kinase inhibition lessens inflammation and ameliorates disease in murine models of hemophagocytic lymphohistiocytosis. Blood. 2016; 127:1666-1675; and Maschalidi S, Sepulveda F E, Garrigue A, Fischer A, de Saint Basile G. Therapeutic effect of JAK1/2 blockade on the manifestations of hemophagocytic lymphohistiocytosis in mice. Blood. 2016.

### Multiparametric Flow Cytometry.

Flow cytometry was performed as previously described.See, e.g., Kenderian S S, Ruella M, Shestova O, et al. CD33 Specific Chimeric Antigen Receptor T Cells Exhibit Potent Preclinical Activity against Human Acute Myeloid Leukemia. Leukemia. 2015.

### Statistical Analysis.

All statistics were performed as indicated using GraphPad Prism 6 for Windows, version 6.04 (La Jolla, Calif.). Details of statistics used in individual experiments are listed in figure legends.

### Results

### Establishment of a Novel CRS Xenograft Model

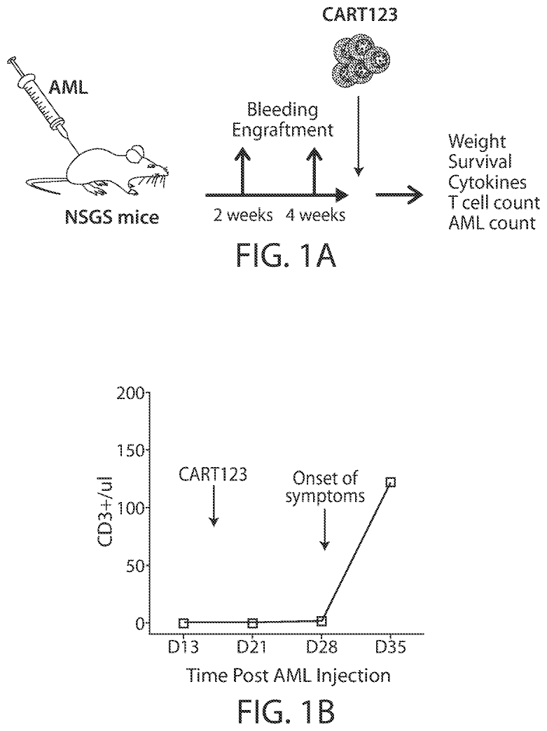

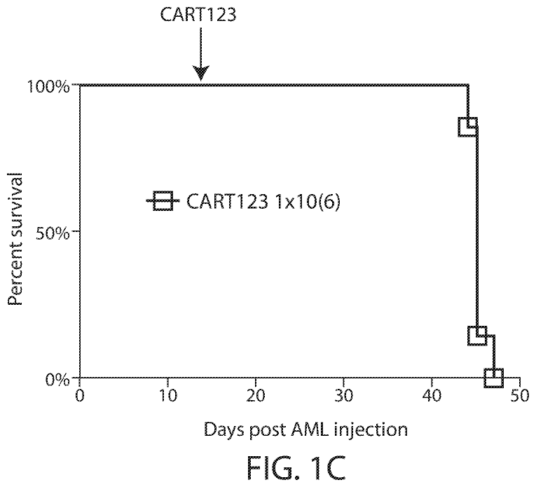

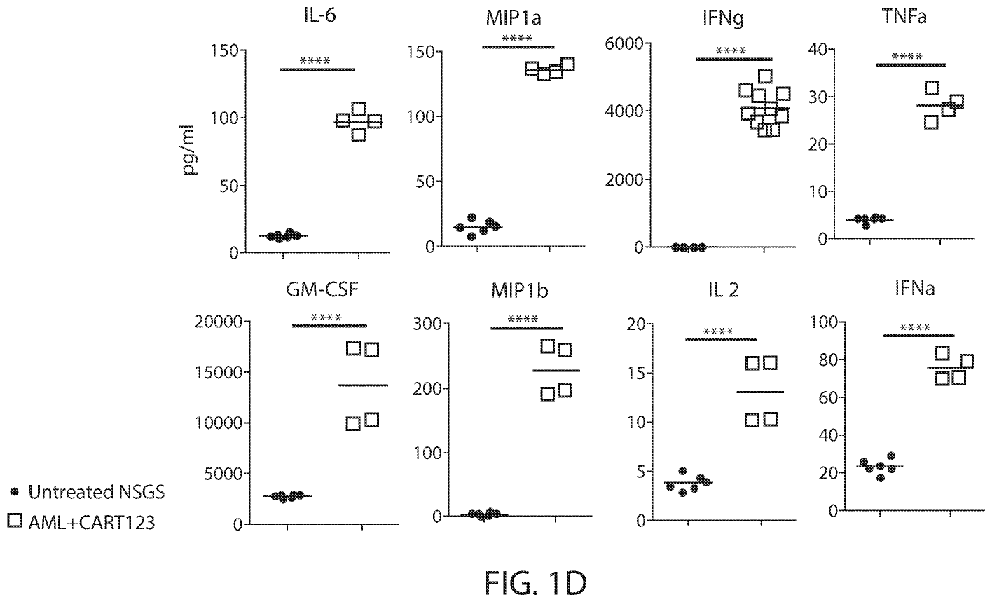

Using NSG-S mice and primary leukemic blasts, a novel AML xenograft model was established to study the development of CRS. NSG-S mice were engrafted with blasts from AML patients and treated with doses of CD123 directed CART cells (CART123) that were ten-fold higher than in previous reports (FIG. 1A). See, e.g., Gill S, Tasian S K, Ruella M, et al. Preclinical targeting of human acute myeloid leukemia and myeloablation using chimeric antigen receptor-modified T cells. Blood. 2014; 123:2343-2354. In particular, NSG-S mice were engrafted with primary AML blasts (5×10⁶) and bled after 2-4 weeks to confirm engraftment. Mice then were treated with high doses of CART123 1×10⁶ by intravenous tail vein single injection and monitored with serial clinical examinations, weight recording and retro-orbital bleedings for leukemia burden assessment and cytokine analysis. These animals developed an illness characterized by progressive weight loss, generalized weakness, emaciation, hunched bodies, withdrawal and poor motor response. This illness started within one week of CART cell injection, correlated with T cell expansion (CART123 expansion in peripheral blood occurred 10-14 days after injection; FIG. 1B). The illness rapidly evolved and resulted in the death of the animals in 5-7 days (FIG. 1C). A high dose of CART123 resulted in early mortality of established AML xenografts (within two weeks of injection). (CART123 were injected on day 41 post AML injection in the experiment.)

Mice were bled for serum cytokines one week after CART123 treatment. Mice treated with CART123 had significant elevation of multiple inflammatory cytokines as outlined in the panel. In particular, serum from these mice five days after CART123 showed an extreme elevation of IL-6, Interferon-γ, tumor necrosis α, and other inflammatory cytokines (FIG. 1D), resembling human CRS after CART cell therapy. See, e.g., Lee D W, Kochenderfer J N, Stetler-Stevenson M, et al. T cells expressing CD19 chimeric antigen receptors for acute lymphoblastic leukaemia in children and young adults: a phase 1 dose-escalation trial. Lancet. 2015; 385:517-528; Kalos M, Levine B L, Porter D L, et al. T cells with chimeric antigen receptors have potent antitumor effects and can establish memory in patients with advanced leukemia. Sci Transl Med. 2011; 3:95ra73; Porter D L, Levine B L, Kalos M, Bagg A, June C H. Chimeric antigen receptor-modified T cells in chronic lymphoid leukemia. N Engl J Med. 2011; 365:725-733; and Grupp S A, Kalos M, Barrett D, et al. Chimeric antigen receptor-modified T cells for acute lymphoid leukemia. N Engl J Med. 2013; 368:1509-1518.

Treatment with Ruxolitinib Ameliorated CRS Severity without Impairing Anti-Tumor Activity after CART123

Ruxolitinib is a JAK/STAT pathway inhibitor that is FDA approved for myelofibrosis and polycythemia vera. See, e.g., Harrison C, Kiladjian J J, Al-Ali H K, et al. JAK inhibition with ruxolitinib versus best available therapy for myelofibrosis. The New England journal of medicine. 2012; 366:787-798; and Vannucchi A M, Kiladjian J J, Griesshammer M, et al. Ruxolitinib versus standard therapy for the treatment of polycythemia vera. The New England journal of medicine. 2015; 372:426-435. In preclinical and clinical studies, ruxolitinib has resulted in a significant reduction of inflammatory cytokines. See, e.g., Quintas-Cardama A, Vaddi K, Liu P, et al. Preclinical characterization of the selective JAK1/2 inhibitor INCB018424: therapeutic implications for the treatment of myeloproliferative neoplasms. Blood. 2010; 115:3109-3117; Das R, Guan P, Sprague L, et al. Janus kinase inhibition lessens inflammation and ameliorates disease in murine models of hemophagocytic lymphohistiocytosis. Blood. 2016; 127:1666-1675; Maschalidi S, Sepulveda F E, Garrigue A, Fischer A, de Saint Basile G. Therapeutic effect of JAK1/2 blockade on the manifestations of hemophagocytic lymphohistiocytosis in mice. Blood. 2016.

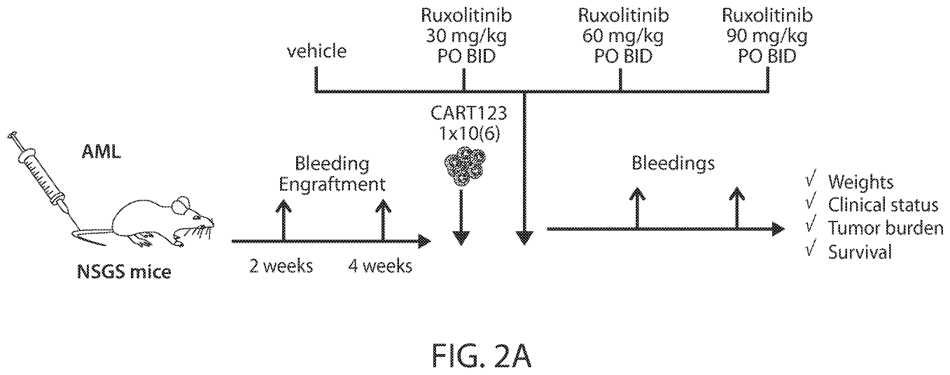

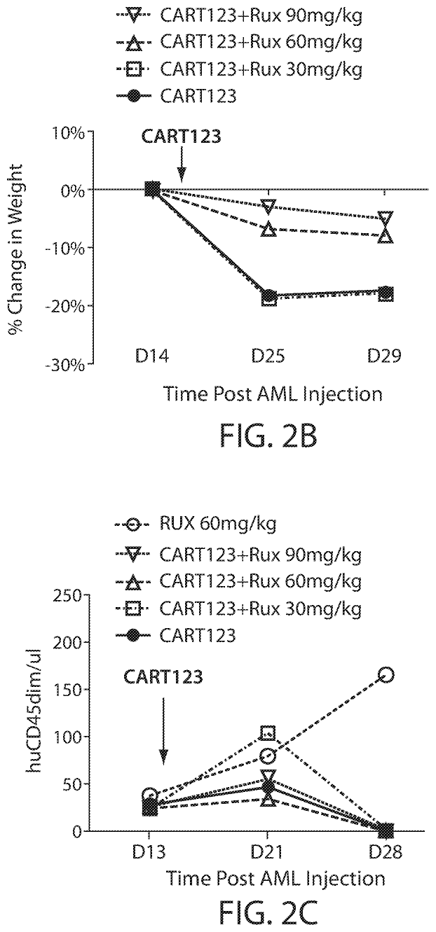

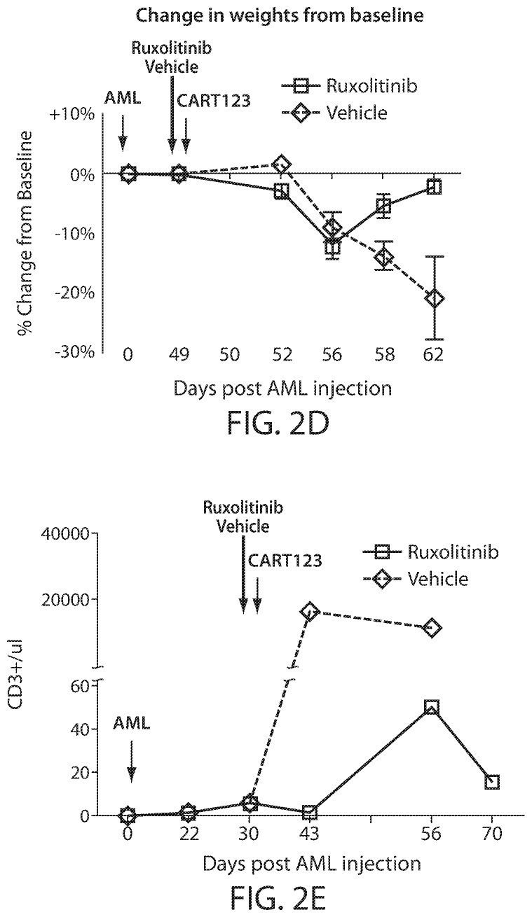

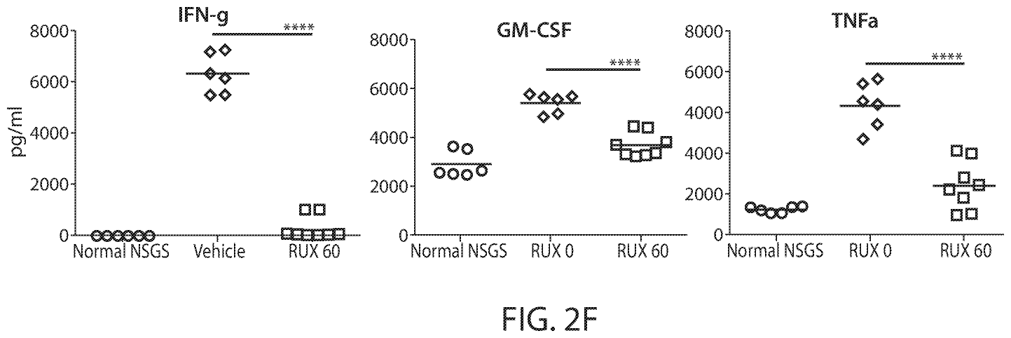

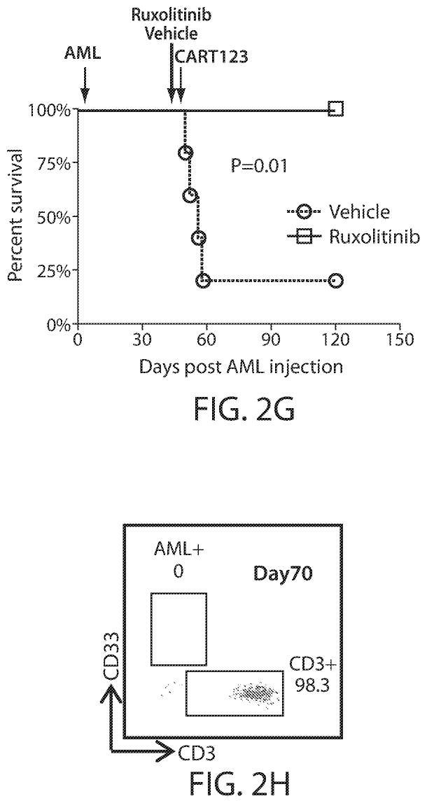

The experiments in this Example investigated ruxolitinib as a modality to prevent or reduce CRS severity after CART123 in the AML xenograft model described herein. NSGS mice were engrafted with primary AML blasts (5×10⁶) and bled after 2-4 weeks to confirm peripheral blood engraftment. NSGS mice bearing primary AML were treated with CART123 (1×10⁶) by intravenous tail vein single injection along with ruxolitinib or vehicle control. The mice were randomized to receive different doses of ruxolitinib (30 mg/kg, 60 mg/kg or 90 mg/kg) or vehicle by oral gavage twice a day. Treatment started on the day of CART123 injection and continued for a week (FIG. 2A). Mice were then monitored with serial clinical examinations, weight recording, retro-orbital bleedings for leukemia burden assessment and cytokine analysis and followed for survival.

Mice treated with ruxolitinib 60 mg/kg or 90 mg/kg exhibited less severe clinical illness (CRS) as manifested by attenuated weight loss when compared with mice treated with CART123 alone or with CART123 combination with ruxolitinib 30 mg/kg (FIG. 2B).

All groups exhibited an equivalent anti-leukemic effect (FIG. 2C). This suggests that ruxolitinib had no direct anti-tumor activity and did not impair the anti-tumor activity of CART123. Therefore, ruxolitinib 60 mg/kg was used for further experiments. Ruxolitinib resulted in amelioration of illness in these mice, transient weight loss (with 60 mg/kg) (FIG. 2d), leukemia eradication (FIG. 2H), and blunted T cell expansion in the peripheral blood (FIG. 2E). Further, ruxolitinib treatment reduced levels of inflammatory cytokines (FIG. 2F) and led to long-term disease free survival (FIG. 2G). In particular, mice treated with high doses of CART123 had early mortality (death due to illness associated with CRS), while mice treated with a combination with ruxolitinib 60 mg/kg led to long term survival. In an analysis of peripheral blood from surviving mice treated with ruxolitinib at 70 days post AML injection (gated on live human CD45 positive cells), all surviving mice had eradication of leukemia. Data are representative of two independent experiments.

These results describe the generation of a clinically relevant animal model of human CRS. The results also demonstrate that the JAK/STAT inhibitor ruxolitinib can prevent the development of severe CRS without impairing the anti-tumor effect of CART cells. The mechanism by which ruxolitinib achieves this effect may be through attenuation of the production of multiple cytokines, including the canonical CRS-inducing cytokines. In the absence of a preclinical model of the CART19/ALL system, these results provide a useful platform for the study of CRS prevention and treatment modalities. Ruxolitinib has been studied clinically for myeloproliferative neoplasms, graft-versus-host disease, and “Philadelphia-like” ALL. See, e.g., Zeiser R, Burchert A, Lengerke C, et al. Ruxolitinib in corticosteroid-refractory graft-versus-host disease after allogeneic stem cell transplantation: a multicenter survey. Leukemia. 2015; 29:2062-2068; and Roberts K G, Li Y, Payne-Turner D, et al. Targetable kinase-activating lesions in Ph-like acute lymphoblastic leukemia. N Engl J Med. 2014; 371:1005-1015. These results herein provide evidence that ruxolitinib can be combined with CART cell therapy for the prevention of CRS.

### Example 2: Ibrutinib Improved Cytokine-Release Syndrome after Anti-CD19 Chimeric Antigen Receptor T Cells for B Cell Neoplasms

Chimeric antigen receptor T cells (CART) hold great promise for the treatment of B cell neoplasms. Anti-CD19 chimeric antigen receptor T cells (CART19) can generate impressive responses, including complete responses, in B cell leukemia and lymphoma. See, e.g., Porter et al. The New England journal of medicine 2011; 365(8):725-33; Maude et al. N Engl J Med 2014; 371(16):1507-17; Schuster et al. Blood 2015; 126(23):183-183; Davila et al. Sci Transl Med 2014; 6(224):224ra25; Turtle et al. J Clin Invest 2016; 10.1172/JCI85309; Lee et al. Lancet 2015; 385(9967):517-28; Kochenzerfer et al. Journal of Clinical Oncology 2015; 33(6):540-9; Dai et al. J Natl Cancer Inst 2016; 108(7). However, widespread applicability of this immunotherapy can be limited by cytokine-release syndrome (CRS).

CRS is a severe systemic inflammation with massive release of cytokines by activated T cells and immune cells, can lead to serious toxicities including deaths. CRS is characterized by elevated cytokines (IFNg, TNFα, IL-6 and others) in the peripheral blood representing a systemic inflammatory state. See, e.g., Kalos et al. Science translational medicine 2011; 3(95):95ra73. Clinically CRS is characterized by high fevers and a systemic inflammatory response that may progress to hypotension, hypoxia, altered mental status, multi-organ dysfunction and death. CRS is observed in the majority of responding patients and typically correlates with high tumor burden. See, e.g., Maude et al. N Engl J Med 2014; 371(16):1507-17. Mitigating strategies that have been attempted include CART19 dose reduction or fractionation and tumor cytoreduction before CART19 infusion. See, e.g., Davila et al. Sci Transl Med 2014; 6(224):224ra25; Turtle et al. J Clin Invest 2016; 10.1172/JCI85309; Frey et al. American Society of Hematology Annual (ASH) Meeting 2014 2014; Abs #2296; Park et al. Journal of Clinical Oncology 2015; 33(15); Lee et al. Blood 2014; 124(2):188-95; and Maude et al. Cancer J 2014; 20(2):119-22. Approaches to prevent CRS have been lacking.

Recently, a novel algorithm has been developed with the aim of predicting CRS and possibly starting pre-emptive treatments. See, e.g., Teachey et al. Cancer Discov 2016; 10.1158/2159-8290.CD-16-0040. However, current practice is to reserve tocilizumab and steroids for patients experiencing severe (grade 3-4) CRS, due to the concern that pre-emptive CRS treatment could impair the anti-tumor effect of the infused CART cells. The management of CRS remains a key factor for extending CART19 to older and frail patients and to increase its safety in the fit adult and pediatric cohorts. (Frey et al. J Clin Oncol 34, 2016 (suppl; abstr 7002)).

The Bruton's tyrosine kinase inhibitor ibrutinib is FDA-approved for relapsing chronic lymphocytic leukemia (CLL) and mantle cell lymphoma (MCL) and is extensively used in B cell neoplasms. See, e.g. Wang et al. Blood 2015; 126(6):739-45; and Byrd et al. N Engl J Med 2013; 369(1):32-42.

Ibrutinib can be combined with CART19, leading to synergistic responses in both MCL and ALL. See, e.g. Ruella et al. Clin Cancer Res 2016; 10.1158/1078-0432.CCR-15-1527; Fraietta et al. Blood 2016; 10.1182/blood-2015-11-679134. Ibrutinib has also been shown to modulate T cell functions. Ibrutinib inhibits IL-2 induced tyrosine kinase (ITK) that is expressed in T and NK cells. See, e.g., Dubovsky et al. Blood 2013; 122(15):2539-49. This effect could lead to modulation of T cell cytokine production as shown in murine T cell models, and to increase the effect of checkpoint blockade together with reduction of cytokine production, as demonstrated in a NK model. See, e.g., Sagiv-Barfi et al. Proc Natl Acad Sci USA 2015; 112(9):E966-72; and Kohrt et al. Blood 2014; 123(12):1957-60. Ruella et al. showed that ibrutinib can blunt cytokine production by CART19 cells in vitro. See Ruella et al. Clin Cancer Res 2016; 10.1158/1078-0432.CCR-15-1527.

Experiments described herein were performed to determine whether adding ibrutinib to CART19 would reduce CRS without impairing the anti-tumor effect, thus enhancing overall survival in a relevant preclinical model.

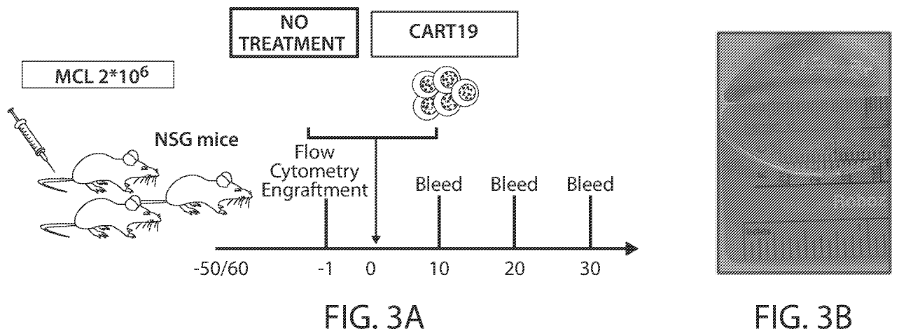

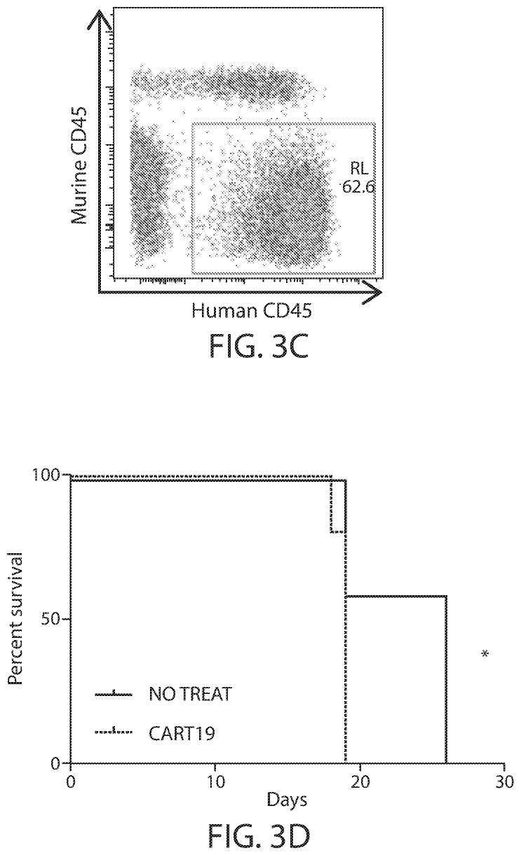

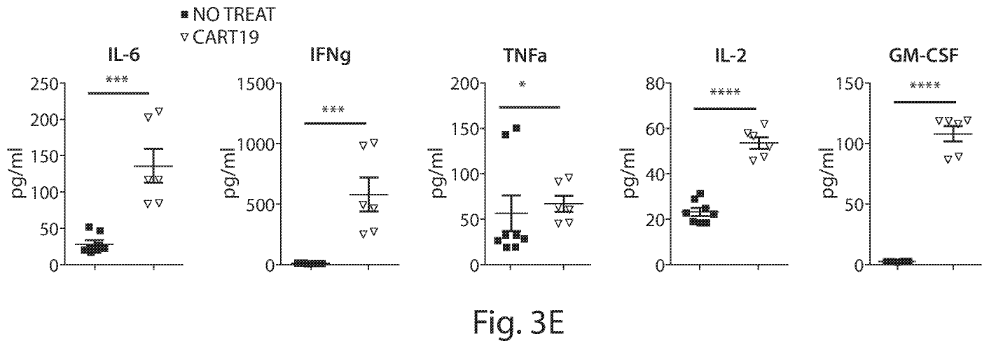

To date, there are no known models that recapitulate the massive increase in cytokine release that is observed after CART-19 treatment. This example describes the development of such a model generated by intravenously injecting NOD-SCID gamma-chain deficient mice (NSG) with primary MCL cells collected from a patient with relapsed MCL (FIG. 3A). NOD SCID Gamma-chain deficient mice were injected with 2×10⁶ primary MCL cells intravenously. Engraftment was monitored with serial retro-orbital peripheral blood bleeding and when neoplastic B cells were detected (high tumor burden, typically around day 50-60) mice were randomized to receive no treatment or CART19. Mice were then followed up for clinical signs, T cell engraftment, tumor burden and survival.

Since CRS has been clearly associated with high tumor burden, the tumor was allowed to grow for up to 50-60 days when spleen size was significantly increased and clinically palpable. High tumor burden was demonstrated by the size of the spleen of a representative mouse sacrificed before T cell treatment (FIG. 3B). At that point in time, point neoplastic B cells were also seen in the peripheral blood (FIG. 3C). At that point 1×10⁶ CART19 cells were injected i.v. As a control, PBS instead of cells was injected. By day 2 after the infusion, mice receiving CART19, but not mice receiving PBS, started to show clinical signs of distress (reduced mobility, hyperventilation) and experienced early death (FIG. 3D) compared to controls (p<0.05). Clinically, this early toxicity resembled CRS. At day 4 after CART19 infusion, serum from mice was collected and analyzed for cytokine concentration by Luminex. The Luminex assay is specific for human cytokines as it does not react with murine cytokines. CART19-treated mice but not control mice showed significantly elevated serum concentrations of several human cytokines, including IL-6, IFNg, TNFα, IL-2 and GM-CSF (FIG. 3E).

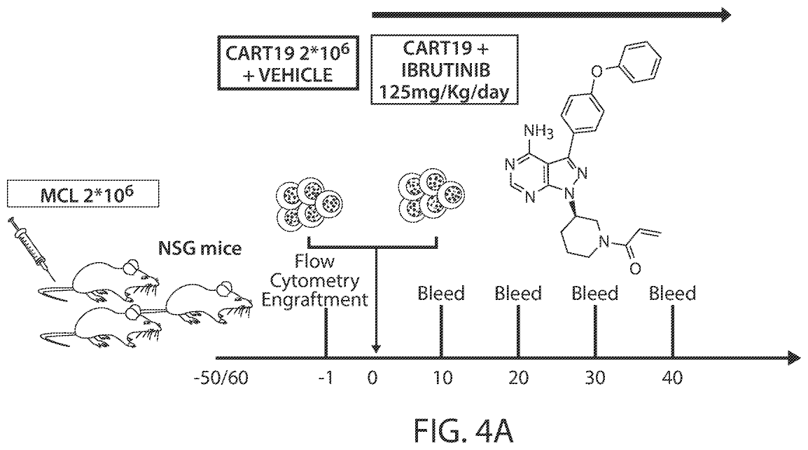

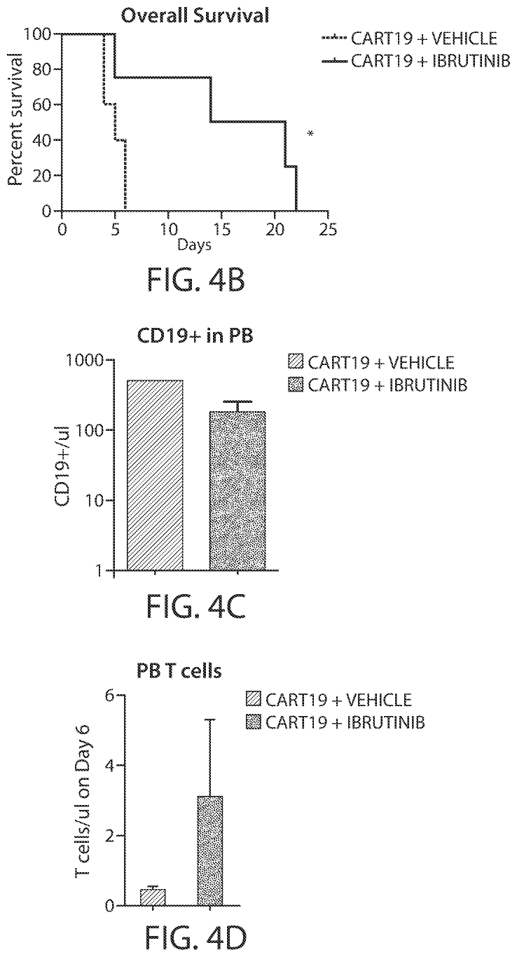

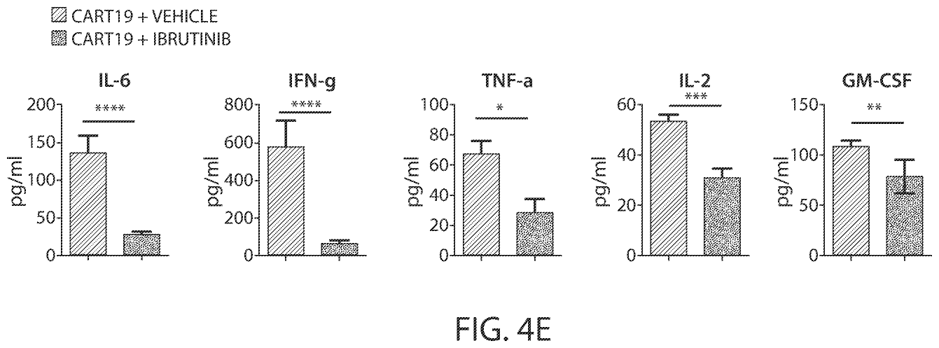

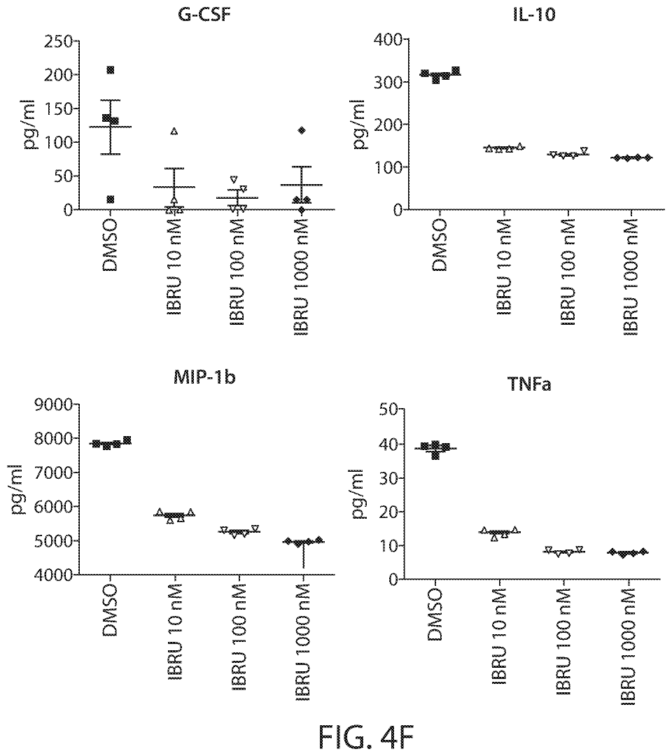

Having established a clinically-relevant model of CRS in the context of B cell neoplasms treated with CART19, experiments were performed to determine if the addition of ibrutinib to CART19 would reduce this early toxicity. NOD SCID Gamma-chain deficient mice were injected with 2×10⁶ primary MCL cells intravenously. Engraftment was monitored with serial retro-orbital peripheral blood bleeding and when neoplastic B cells were detected (high tumor burden, typically around day 50-60), mice were randomized to receive CART19 plus vehicle or CART19 plus ibrutinib in the drinking water (125 mg/Kg/day). Ibrutinib (PCI-32765) was purchased from MedKoo (#202171) or Selleck Biochemicals (#S2680) as a powder or DMSO solution. The products obtained from the two companies were compared and demonstrated to have equivalent activity (data not shown). For in vitro experiments, ibrutinib was dissolved in DMSO and diluted to 2, 10, 100 or 1000 nM in culture media. For in vivo experiments, ibrutinib powder was dissolved in a 10% HP-beta-cyclodextrin solution (1.6 mg/ml) and administered to mice in the drinking water. Mice were then followed up for clinical signs, T cell engraftment, tumor burden and survival. As shown in the schematic in FIG. 4A, high-tumor burden mice were treated with either CART19 (plus vehicle) or CART19 in combination with ibrutinib. Mice receiving the combination of CART19 and ibrutinib had a prolonged overall survival (OS) as compared to mice receiving CART19 alone (FIG. 4B), despite similar tumor burden (FIG. 4C). Also, ibrutinib augmented, rather than impaired, CART19 expansion in the PB (FIG. 4D)—this confirmed previous observations. See, e.g., Ruella et al. Clin Cancer Res 2016; 10.1158/1078-0432.CCR-15-1527. Cytokines in the peripheral blood at day 4 were significantly reduced in ibrutinib-treated mice, including IL-6, IFNg, TNFα, IL-2 and GM-CSF (FIG. 4E).

Having previously shown that ibrutinib led to a modest reduction in T cell cytokine production (see I.d.), and given that ibrutinib was initially developed as a cytostatic anti-tumor agent, experiments were performed to determine whether ibrutinib treatment affects cytokine production by cultured MCL cells as well. As shown in FIG. 4F, in vitro increasing concentrations of ibrutinib reduced the cytokines produced by the tumor, possibly contributing to the reduced CRS observed in vivo.

CRS is the major factor limiting the widespread feasibility of CAR T cell therapy for cancer. The results herein demonstrate development of a relevant pre-clinical model for CRS (e.g., fatal CRS) in B cell neoplasms after CART19 treatment. Elevated levels of human inflammatory cytokines were found in the serum of CART19-treated mice as compared to controls. Co-administration of ibrutinib with CART-19 blunted this cytokine storm and significantly increased overall survival (p<0.05). The data herein show that the BTK/ITK inhibitor ibrutinib in combination with CART19 led to a reduction in CRS and enhanced survival by blunting the production of inflammatory cytokines from both CART and tumor cells. The results show that ibrutinib did not impair T cell proliferation in vivo, a factor that has been shown to be a key element for anti-tumor efficacy. Ibrutinib synergizes with the anti-tumor efficacy of CART19 in B-cell malignancies. The results presented herein suggest that the combination of ibrutinib and CART19 can reduce the toxicity of CAR T cell therapy. The CART19-ibrutinib combination may be a new strategy to prevent CRS in acute leukemia as well as an attractive two-pronged approach for B cell neoplasms that are currently treated with ibrutinib.

### Example 3: Chimeric Antigen Receptor T Cell Activation Induced Interleukin 6 Secretion by Monocyte-Lineage Cells

Chimeric antigen receptor T cell therapy targeting CD19 has demonstrated success against B-cell malignancies, but is sometimes complicated by serious systemic toxicity in the form of cytokine release syndrome (CRS). The symptoms of this syndrome appear to be primarily mediated by elevations in interleukin 6 (IL-6), and management has focused on inhibition of IL-6 signaling. The cellular source and function of IL-6 in CRS remained unknown before this study; this has limited informed management of CRS. The results herein demonstrate that secretion of IL-6 is driven by CAR T cell activation but is derived from monocyte-lineage APCs. T cell-induced activation of APCs occurred in a contact-independent mechanism, and IL-6 secreting APCs had no impact on T cell transcriptional profiles or cytotoxicity. The results herein also show that CAR T cells delivered to patients with acute lymphoblastic leukemia did not secrete IL-6 in vivo during clinical CRS. These results suggest that anti-IL-6 therapy may not impact CAR T cell anti-tumor efficacy.

### Introduction

The primary toxicity associated with highly-active cellular therapy using CD19 chimeric antigen receptor (CD19 CAR) T cells is the hyper-inflammatory state known as “cytokine release syndrome” (CRS) (Grupp NEJM 2013). This toxicity is characterized by clinical symptoms ranging from a mild influenza-like syndrome to extreme elevations in core body temperature and life-threatening multi-organ failure. In a report of CD19 CAR T cells for acute lymphoblastic leukemia (ALL), Grupp et al. described a biochemical profile demonstrating significant elevations in several serum cytokines, including interleukin-2 (IL-2), interleukin-6 (IL-6) and gamma-interferon (INF-7) in patients with CRS (Grupp NEJM 2013). One patient treated in a phase I study experienced dramatic toxicity in the form of distributive shock requiring multiple vasopressors for vascular support and respiratory failure requiring prolonged mechanical ventilation. Administration of the anti-IL-6 receptor agent tocilizumab several days into CRS resulted in prompt hemodynamic stabilization, pointing to the central role of IL-6 in causing these symptoms. Davila et al. and Lee et al. have reported a similar IL-6-driven syndrome with CD19 CAR T cells (Davila STM 2014, Lee Lancet 2015). There is limited understanding of the cellular source of IL-6 during CRS, whether IL-6 is secreted by CAR T cells themselves as a means of homeostatic support in a rapidly dividing cell population, or if IL-6 is necessary for T cell activity. Several cellular sources of IL-6 have been identified, including macrophages, dendritic cells and B and T lymphocytes (Schulert and Grom, Ann Rev Med 2015; Leech M D J I 2013; Barr T A J E M 2012; Trinschek Plos One 2013). T cells have been identified as the primary source of pathologic IL-6 in models of multiple sclerosis (Trinschek Plos One 2013), and T cell-derived IL-6 has been implicated in mediating a positive feedback loop driving T_{H}17 cell differentiation (Ogura Immunity 2008), allowing for the possibility that T cells themselves are the source of the high level of IL-6 observed. While classical T cell activation in response to infection and auto-antigens has been studied, the effect of the mechanism of T cell activation is poorly understood in the context of CARs, and CAR-driven activation may generate distinct cytokine support needs and have a distinct effect on T cell-mediated IL-6 production. In the absence of a better understanding of the role of IL-6 in CAR T cell function and in CRS, balancing the management of severe toxicity with optimization of anti-tumor activity has been driven by empiric trial and error.

In an examination of a panel of serum cytokines in pediatric and adult patients receiving CD19 CAR T cell therapy for ALL, Teachey et al. observed that elevations in IFN-γ, IL-6, IL-8, soluble IL-2 receptor-α (sIL-2Rα), soluble IL-6 receptor (sIL-6R), monocyte chemoattractant protein 1 (MCP1), macrophage inflammatory protein 1α (MIP-1α), macrophage inflammatory protein 1β (MIP-1β) and granulocyte-macrophage colony stimulating factor (GM-CSF) were associated with the development of severe CRS (Teachey Cancer Discov 2016). Early elevations in IFN-γ, serum glycoprotein 130 (sgp130), a component of sIL-6R, and sIL-1RA were predictive markers of development of severe CRS. See Id. Both T-cell expansion and baseline disease burden have been thought to be primary determinants of severity of CRS; however T-cell expansion itself was not associated with development of CRS. Similarly, disease burden alone did not provide any further predictive modeling over serum cytokine levels. Examination of all patients who received tocilizumab therapy demonstrated a consistent and rapid resolution of toxicity after administration, with discontinuation of vasopressors within 24-36 hours, confirming the clinical significance of IL-6 in mediating toxicity. See Id.

The immunologic cascade that results from CAR-mediated T cell activation, as opposed to native TCR-mediated activation, and the resulting cellular events that lead to the biochemical derangements of CRS have clinical relevance; two adult patients treated at the University of Pennsylvania have died while experiencing CRS, and many patients have experienced significant morbidity. The Teachey et al. study described above provided a detailed cytokine profile of patients who experienced CRS, and it was observed that cytokine dynamics in CRS are almost identical to those in hemophagolytic lymphohistiocytosis (HLH). This inflammatory syndrome is driven by macrophage activation, suggesting that CAR T cells are unlikely lone actors in CRS, and other immune cells may be key players. The clinical understanding thus far has hinged on elevations in IL-6; IL-6 drives clinical symptoms, and IL-6 is one of several cytokines that becomes upregulated during CAR T cell activation in vivo, suggesting a network of cytokine signaling contributing to CRS.

In this Example, to investigate the cellular drivers of CRS, antigen-presenting cells (APCs) derived from the monocyte lineage were isolated. In vivo and in vitro co-culture experiments were performed to identify which cell types led to the cytokine elevations associated with this clinical syndrome. The results herein demonstrate that while T cells alone are sufficient for the production of some CRS-associated cytokines, both activated T cells and APCs are necessary for production of IL-6, and that this dependence is not reliant on cell:cell contact. The results herein also identify that monocyte-derived cells are responsible for IL-6 secretion in response to CAR-mediated T cell activation, and that CAR-activated T cells are not affected by the presence of APCs. These details of the CRS cascade may provide not only a deeper immunologic understanding of this syndrome, but also further opportunity for management of CRS.

### Materials and Methods

### Xenograft Studies and Patient Samples

6-10 week old NOD-SCID-γc^{-/-} (NSG) mice were obtained from the Jackson Laboratory (Bar Harbor, Me.) or bred in house under an approved Institutional Animal Care and Use Committee (IACUC) protocol and maintained in pathogen-free conditions. Patient leukemia and T cells were obtained under a Children's Hospital of Philadelphia Institutional Review Board approved protocol (CHP959 and CHP784, respectively). T cell engineering for this study has been described previously (Grupp et al NEJM 2013). Animals were given 10⁶ primary human ALL cells via tail vein, followed by 5×10⁶ CAR T cells (11% CAR+) seven days later. Peripheral blood was collected via retro-orbital sinus and submitted to the University of Pennsylvania Human Immunology Core for cytokine quantification.

### Isolation of Normal Donor Monocytes and T Cells and T Cell Engineering

Primary human T cells and monocytes from normal donors were procured through the University of Pennsylvania Human Immunology Core. For all co-culture experiments, T cells and monocytes were obtained from the same donor. T cells were combined at a ratio of 1:1 CD4:CD8 cells at a concentration of 10⁶ cells/mL T cell culture media with stimulatory microbeads coated with antibodies directed against CD3 and CD28 (Life Technologies, Grand Island, N.Y., Catalog #111.32D) at a concentration of 3 beads/cell, as had been reported previously (Laport G G, Blood 2003). 24 hours after initial stimulation, T cells were exposed to lentiviral vector encoding the CD19 CAR construct at a multiplicity of infection (MOI) of 5-10 particles/cell. Stimulatory beads were removed on day 7, and cells were counted and volumes measured serially until growth and size trends indicated cells were rested down, at which time they were frozen. Cells were then thawed 12-18 hours prior to in vivo injection or in vitro co-culture. Untargeted T cells were cultured in the same manner but were not treated with lentiviral vector.

### Lentiviral Vector Preparation

High-titer, replication-defective lentiviral vectors were produced using 293T human embryonic kidney cell. HEK293T cells were seeded at 10⁷ cells per T150 tissue culture flask 24 hours before transfection. On the day of transfection, cells were treated with 7 μg of pMDG.1, 18 μg of pRSV.rev, 18 μg of pMDLg/p.RRE packaging plasmids and 15 μg of transfer plasmid in the presence of either Express-In Transfection Reagent (Open Biosystems, Lafayette, Colo.) or Lipofectamine 2000 transfection reagent (Life Technologies, Grand Island, N.Y., Catalog #11668019). Transfer plasmids containing CAR constructs were modified so that expression of the CAR was under control of the EF-1α promoter as previously described. Viral supernatants were harvested 24 hours and 48 hours after transfection and concentrated by ultracentrifugation overnight at 10,500×g. 24h after initial stimulation, T cells were exposed to lentiviral vector at a concentration of 5-10 infectious particles per T cell, and then cultured as described above.

### Production of Monocyte Lineage Cells

Monocytes were collected as described above, and differentiation was performed using methods described previously (Han J Immunother 2009). Briefly, 2×10⁶ monocytes were plated in 1 mL of RPMI 1640 supplemented with supplemented with 0.1 mM MEM Non-Essential Amino Acids, 2 mM L-Glutamine, 100 units/ml Penicillin, 100 μg/ml Streptomycin (Life Technologies) and 10% fetal calf serum and cultured for 4 days. Cells were then harvested using 2 mM EDTA and stained with CD14, CD45, CD68 and CD163 to confirm macrophage differentiation. To produce dendritic cell lineages, monocytes were plated at 6×10⁶ in 1 mL of cR10, and treated with 0.2 μg/mL human IL-4 (R&D Systems, Minneapolis, USA, #204-IL-050) and 0.2 μg/mL GMCSF (R&D Systems, Minneapolis, USA, #215-GM-050). On day 4 cells were either harvested using 2 mM EDTA (immature dendritic cells), or cultures were treated with 100 ng/mL LPS (Sigma-Aldrich, St. Louis, USA; #L2630) and 0.05 μg/mL IFN-γ (R&D Systems, Minneapolis, USA, #285-IF-100) (mature dendritic cells). 24 hours later, cells were harvested with 2 mM EDTA and stained with CD45, CD80 and CD86 to confirm immature DC and mature DC differentiation.

### Co-Culture Assay

T cells were engineered as described above, and monocytes lineages were differentiated as described above. Nalm-6 ALL cell lines were used as targets. Cells were combined at a ratio of 50 T cells, 10 targets and 1 APC in 150 μL of cR10. 20 μL of supernatant was aspirated after 18 hours and replaced with 20 μL cR10. 20 μL was then aspirated again at 48 hours. For trans-well co-culture assays, T cells and targets were cultured as described for our standard co-culture assay. Pooled monocytes were seeded in ThinCert cell culture inserts (Greiner Bio-one) placed in each well of the 24-well plate. Co-cultures were incubated at 37° C. and cells from both the inserts and the wells were collected at 18 hours and 48 hours for RNA isolation as described below.

### Measurement of Cytokine Levels

Cytokine concentration determination from animal serum and from culture supernatants was performed by the University of Pennsylvania Human Immunology Core using the Millipore Luminex 200 system and Milliplex Human Cytokine/Chemokine 21 Plex Assay (EMD Millipore, Bedford, Mass., USA; products #40-012 and #HCY4MG-64K-PX21). Measurements were performed using standard product protocols.

### RNA Extraction

Total RNA was prepared from cell pellets lysed in Qiagen Buffer RLT (Qiagen Inc.).

Lysates were processed and RNA extracted using RNA Clean & Concentrator-5 columns (Zymo Research Corp.) according to the manufacturer's protocol. Total RNA quality and yield were assessed using either an Agilent 2100 Bioanalyzer with Eukaryotic Total RNA Pico chips (Agilent Technologies) or a Biophotometer (Eppendorf) equipped with a Hellma TrayCell microvolume ultra-micro cell (Hellma Analytics).

NanoString nCounter Assay

Gene expression was measured on the nanoString nCounter SPRINT Profiler (NanoString Technologies) using the nCounter Human Immunology v2 gene expression Code Set (NanoString Technologies). Samples were prepared and processed according to the manufacturer's recommendations. Briefly, 50 ng total RNA was hybridized in solution to the nCounter Human Immunology v2 gene expression Code Set for 18 h at 65° C. Hybridized samples were then loaded into the nCounter SPRINT cartridge (NanoString Technologies) which was then sealed and placed in the instrument for processing and analysis.

### CD107a Degranulation Assay

Co-culture experiments were setup as described above. After 18 hours, this culture was combined with an antibody cocktail consisting of anti-CD107a-e660 (eBiosciences, San Diego, Calif., Catalog #50-1079), and stimulatory antibodies directed against CD28 (clone 9.3) and CD49d (BD Biosciences, Franklin Lakes, N.J., Catalog #555051) for one hour. Intracellular protein transport was halted by addition of GolgiStop (BD Biosciences, Franklin Lakes, N.J., Catalog #554724) and cells were incubated for an additional three hours. Cells were then harvested and stained for CD8 and CD107a (BD Biosciences, Franklin Lakes, N.J.) and analyzed on an Accuri C6 Flow Cytometer.

### Results

### Combining CD19 CAR T Cells and Targets Did not Mimic Clinically-Observed CRS in Xenograft Mice

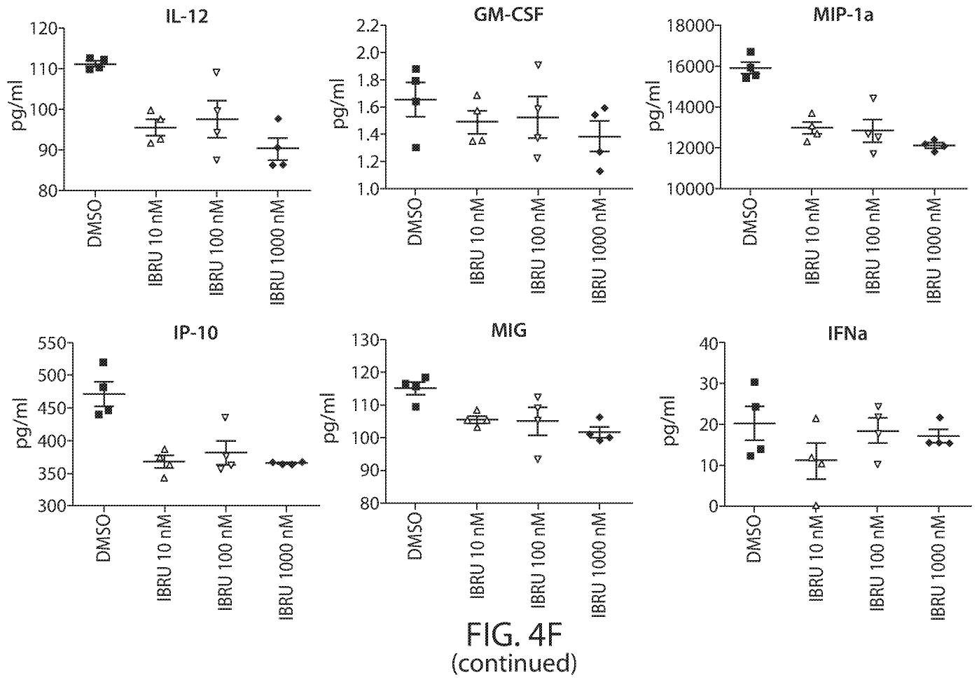

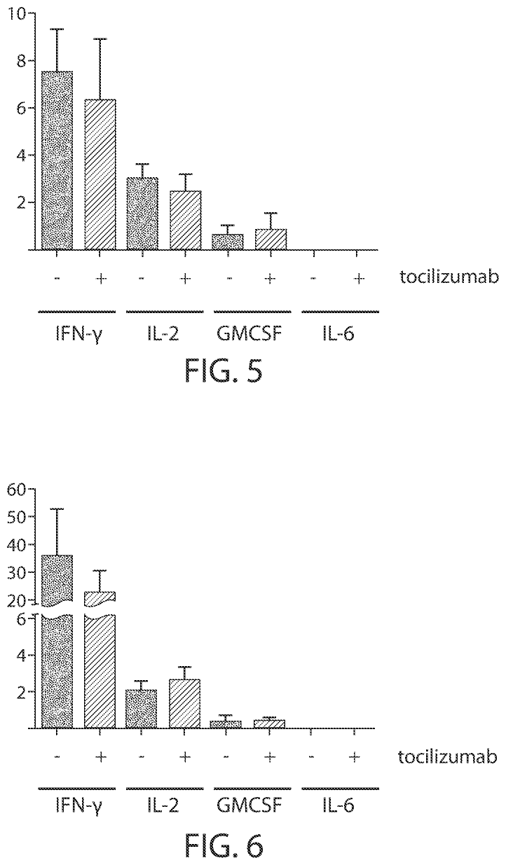

To evaluate the role of CAR-activated T cells in CRS, a patient-derived xenograft model was created of an aggressive and multiply refractory pediatric acute lymphoblastic leukemia (ALL). The malignant cells used to establish this xenograft were derived from a patient with ALL treated as described in Grupp NEJM 2013 (patient CHP-100). As reported in Grupp et al., this patient experienced grade 4 toxicity, including need for prolonged vasopressor support and mechanical ventilation. Clinical CRS was accompanied by a significant elevation in serum IL-6 (approximately 1000× increase on day 6 after CD19 CAR T cell infusion compared to baseline), and rapid resolution of symptoms after administration of αIL6R antibody therapy. To evaluate the role of this patient's CAR T cells in producing CRS-associated cytokines in vivo, NOD/SCID/cγ^{-/-} (NSG) mice were engrafted with 10⁶ primary acute lymphoblastic leukemia cells from patient CHP-100, followed by injection of 5×10⁶ CD19 CART cells from the same patient seven days later (T cells were 11% CAR+). A subgroup of animals was also given tocilizumab (100 μg via intraperitoneal injection) every other day following CAR T cell infusion. Measurement of serum cytokine levels on day 3 after CAR T cell infusion demonstrated measurable levels of IFN-γ, IL-2 and GMCSF, but no detectable IL-6, regardless of the presence of tocilizumab (FIG. 5). A similar pattern was observed when animals were engrafted with the Nalm-6 ALL cell line and treated with CD19 CAR T cells derived from a normal donor (FIG. 6), supporting that this lack of IL-6 production was not a patient-specific phenomenon, and suggesting that the cellular component responsible for the significant IL-6 production observed clinically was not present in these immunodeficient xenografts.

### Presence of APCs During Antigen-Mediated T-Cell Killing Resulted in Elevated Levels of CRS-Associated Cytokines

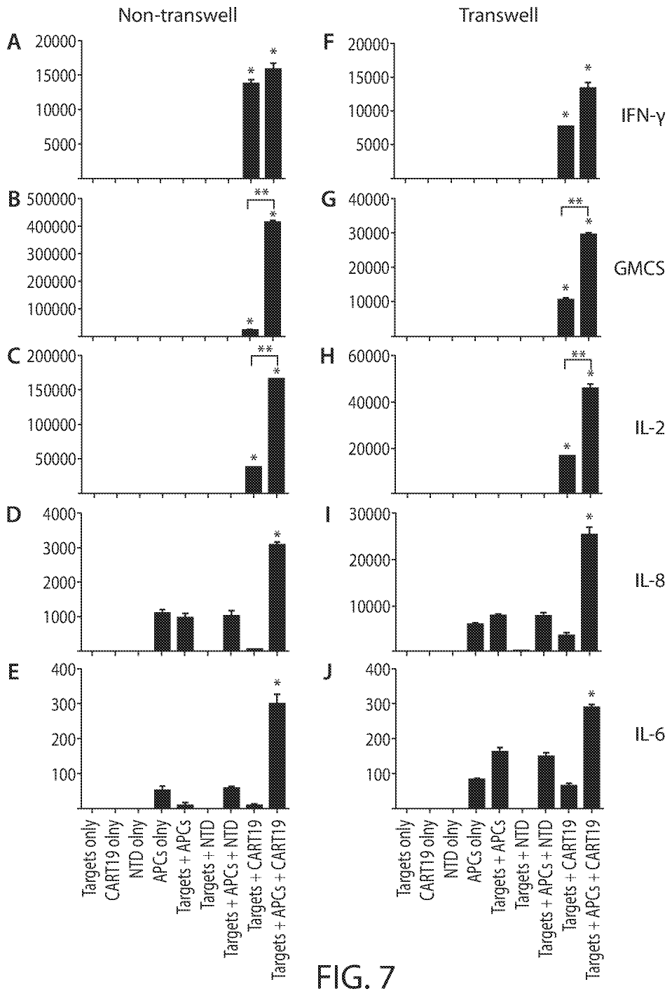

Based on the similarities in serum cytokine profiles between CRS and HLH, the role that antigen-presenting cells of the monocyte lineage may play in cytokine production was evaluated. Either CD19 CAR T cells or untargeted T cells were combined with a CD19+ ALL cell line (Nalm-6) in the presence of APCs in vitro at cell ratios of 10 T cells: 50 targets: 1 APC. Culture supernatants were collected after 18 hours of co-culture. As demonstrated in the prospective clinical study described in Teachey et al. Cancer Discovery 2016, early elevations were observed in serum levels of IFN-γ. IFN-γ was detected whenever T cells were activated by targets, with no significant difference based on the presence of APCs (FIG. 7A). Moderate levels of GMCSF were secreted when T cells were combined with targets, however significant elevations were noted when APCs were included in co-culture (FIG. 7B), suggesting either enhancement of T cell-based secretion by APCs or two cellular sources of GMCSF. IL-2, classically considered a CD4 T cell factor, demonstrated a similar pattern, with some secretion when T cells and targets were combined, but significant enhancement with the inclusion of APCs in co-culture (FIG. 7C). This pattern differed when examining IL-8 and IL-6. Similar levels of IL-8 were observed in cultures of APCs alone, APCs combined with targets and APCs combined with targets and untargeted T cells (FIG. 7D). Levels significantly increased when APCs were combined with targets and targeted T cells (FIG. 7D). Together, these data suggest that APCs secreted low-levels of IL-8 independent of T cells or targets, but the combination of targets, targeted T cells and APCs resulted in high levels of IL-8. IL-6 levels followed a similar pattern (FIG. 7E); low levels were observed when APCs were alone, combined with targets and combined with untargeted T cells and targets. Levels significantly rose when APCs were combined with activated T cells and targets.

To clarify if the APC:T cell interaction was mediated by cell:cell contact or by soluble factors, the same co-cultures were set up in parallel with co-cultures which separated T cells and targets from APCs using trans-well inserts. The same numbers of T cells and targets were placed in the plate wells, and the same number of APCs were placed in trans-well inserts. As shown in FIGS. 7F-7J, absolute concentrations of cytokines varied but relative quantities of cytokine secretion were unchanged for IFN-γ, GMCSF, IL-2 and IL-8. The trans-well separation resulted in a relative increase in IL-6 when APCs were combined with targets in the presence or absence of untargeted T cells. However, the highest IL-6 levels were still observed when CD19 CAR T cells were combined with targets and APCs, demonstrating a statistically significant elevation compared to all other co-culture experiments (p=0.001). These studies demonstrate that the cytokine network induced upon CAR-mediated T cell activation in the presence of APCs was not dependent on cell:cell contact between T cells and APCs, and the combination of targets, CAR T cells and APCs was necessary for high levels of IL-6 production.

### Monocyte-Lineage Cells Differentially Secrete CRS-Associated Cytokines

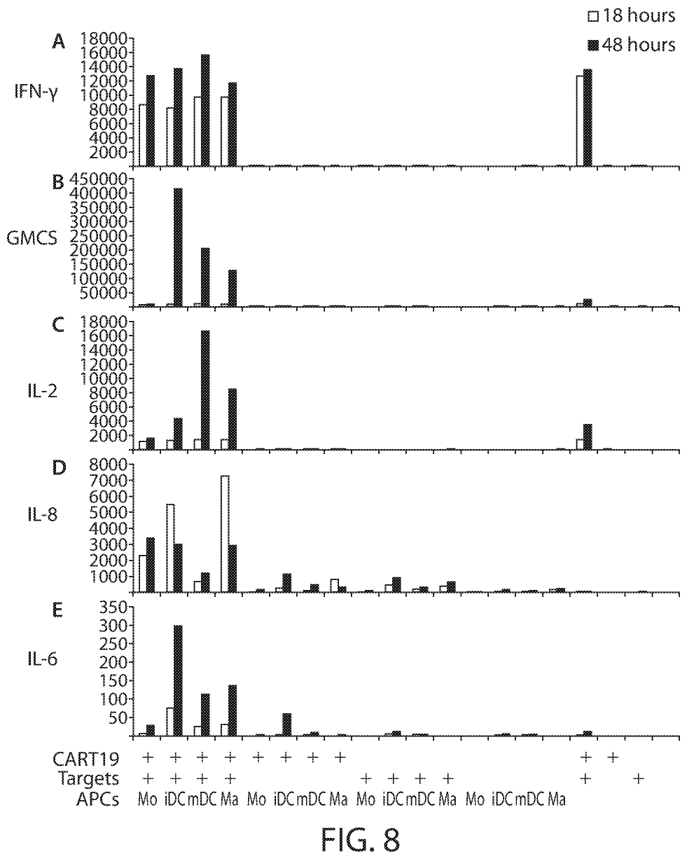

Experiments were performed to identify which APC lineages were necessary for IL-6 secretion. Monocytes were isolated and cultured in vitro to produce the differentiated progeny of the monocyte lineage, namely immature dendritic cells, mature dendritic cells and macrophages (Han J Immunother 2009) (osteoclasts were not included). These undifferentiated monocytes and differentiated lineages were combined with CD19 CAR T cells and targets in co-culture as described. Culture supernatants were collected at 18 and 48 hours. Consistent with findings from FIGS. 7A-7J, IFN-γ levels were elevated in the presence of each APC lineage, as well as in the absence of APCs, suggesting that IFN-γ was produced by activated CAR T cells independent of APCs (FIG. 8A). Levels increased marginally after 48 hours of culture. GMCSF levels demonstrated a significant increase between the 18 and 48-hour time points, with cultures containing immature dendritic cells producing the most GMCSF, followed by mature DCs and then macrophages (FIG. 8B). Activated T cells alone produced very little GMCSF, as did cultures of activated T cells with monocytes, suggesting that GMCSF secretion was driven by differentiated monocyte-lineage cell populations. IL-2 levels demonstrated a large peak at the 48-hour time point when activated T cells were combined with mature dendritic cells, and a smaller but significant peak when T cells were combined with macrophages (FIG. 8C). Low-levels of IL-2 were detected after 48 hours when T cells were combined with targets alone or in the presence of immature dendritic cells, but the presence of monocytes did not appear to result in significant IL-2 production. While some low-level IL-8 was detected in nearly all cultures containing APCs, a 1000× increase was detected when activated T cells were combined with macrophages as compared to T cells and targets alone at the 18-hour time point (FIG. 8D). The presence of immature dendritic cells also yielded high IL-8 levels at 18 hours, with more modest elevations in the monocyte cultures. The presence of mature dendritic cells did not significantly alter IL-8 concentration. Finally, IL-6 levels were observed to be highest when activated T cells were combined with immature dendritic cells after 48 hours of culture, with a greater than 100× increase in cytokine concentration (FIG. 8E). Modest elevations were detected in cultures containing activated T cells and mature DCs and macrophages. Nearly no IL-6 was detected in the absence of APCs, but a low level was produced in the absence of targets (T cells and immature DCs alone); these levels were several logs below those observed when CAR T cells were combined with targets and APCs.

### APC-Produced IL-6 Did not Impact CAR T Cell Transcription or Cytotoxicity

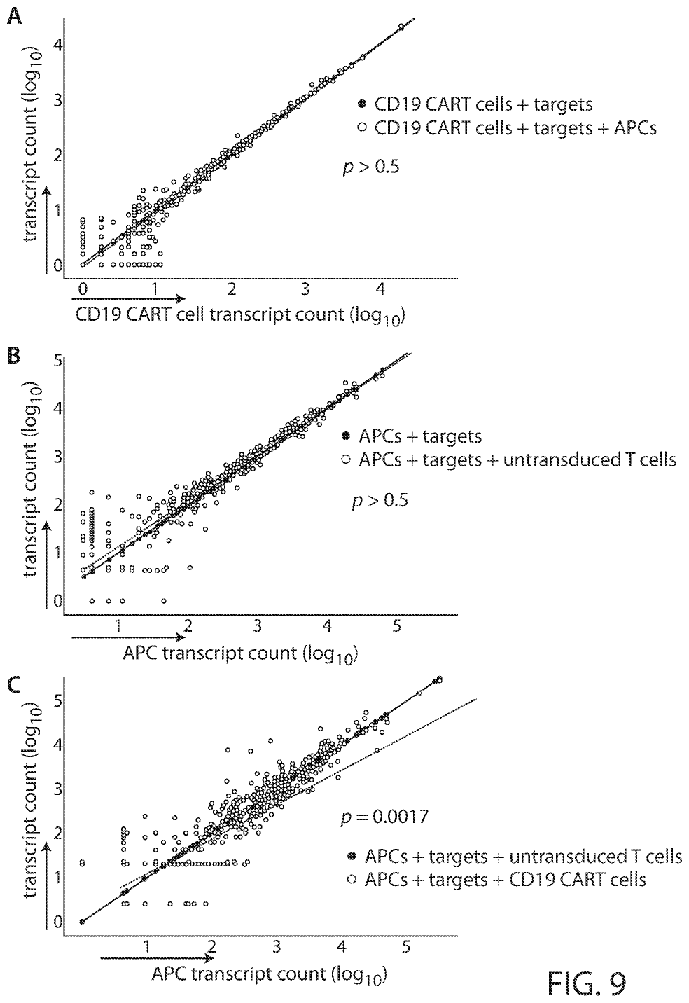

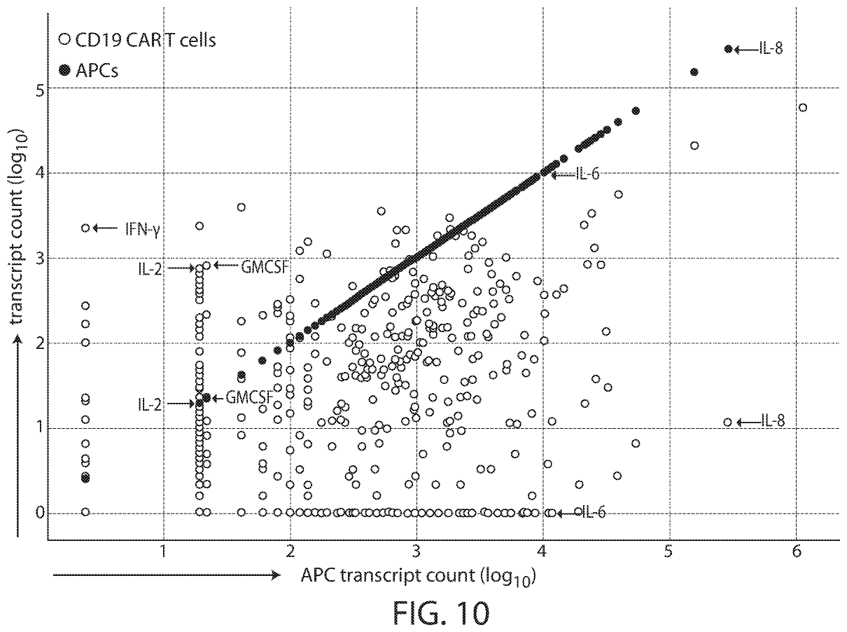

Experiments were performed to determine which cell type (APC or CAR T cell) was responsible for cytokine secretion in these co-cultures. Trans-well co-culture experiments were conducted, and Nanostring transcriptional analysis was performed on discrete cell populations. The transcriptional profiles of activated T cells in the presence or absence of APCs were examined. As depicted by regression analysis of 697 genes related to immune activation, there was no detectible difference in transcriptional profile (FIG. 9A, R²=0.951, p>0.5). The profiles of APCs alone to APCs combined with untargeted T cells and Nalm-6 leukemia were also examined. There was no change in APC transcriptional profile (FIG. 9B, R²=0.934, p>0.5). Additionally, APC transcription profiles when combined with untargeted T cells and Nalm-6 or CD19 CAR T cells and Nalm-6 were compared. There was significant variability in APC transcriptional profiles (FIG. 9C, R²=0.830, p=0.0017). These data demonstrate that APCs had no effect on T cell transcription, but that CAR-activated T cells and not un-activated T cells significantly altered APC transcriptional phenotypes. From the same trans-well studies, Nanostring analysis was used to map RNA constructs to their cell of origin. IFN-γ was made exclusively by T cells, IL-2 and GMCSF were predominantly made by T cells, IL-8 was predominantly made by APCs, and IL-6 was exclusively made by APCs (FIG. 10), confirming the cellular origins of these CRS-associated cytokines.

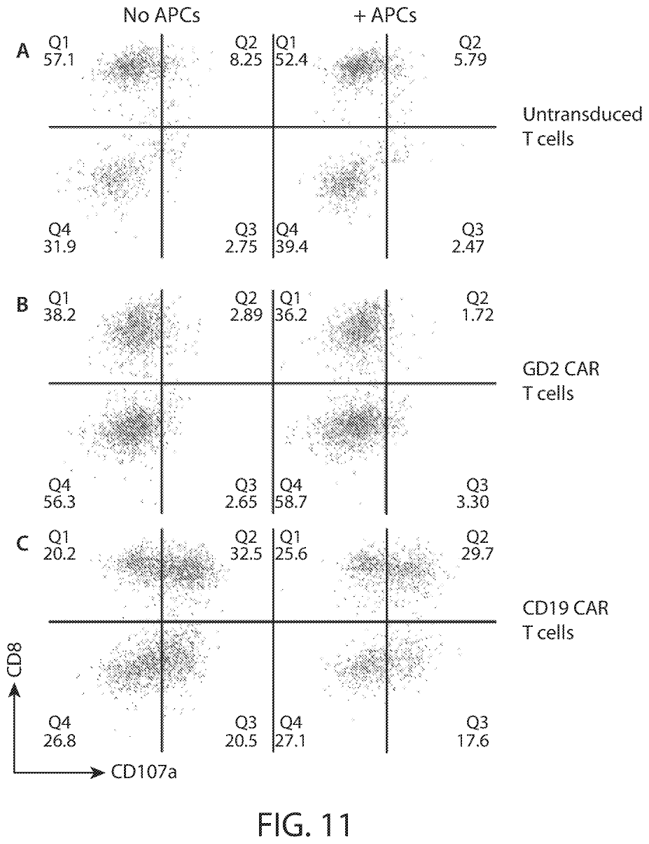

To evaluate if CD19 CAR T cell activity was altered by IL-6 in a transcription-independent manner, co-culture experiments were performed and T cell cytotoxic activity was evaluated. Targets, APCs and T cells were combined as described above and T cells were harvested after 18 hours of co-culture. To control for the effects that non-specific CAR signaling may have on cytotoxicity assessment, T cells were engineered to express either no CAR (FIG. 11A), a CAR directed at the irrelevant antigen GD2 (FIG. 11B) or the CD19 CAR (FIG. 11C). Cytotoxicity was measured by upregulation of CD107a, a measure of T cell degranulation. No degranulation was detected when untargeted T cells or GD2 CAR T cells are combined with targets, while degranulation was detected when CD19 CAR T cells encounter CD19+ leukemia. There was no detectable difference in degree of degranulation based on the presence or absence of APCs.

### Transcriptional Analysis of Clinical CD19 CAR T Cell Samples Revealed Distinct Clustering of Grade 2-3 Versus Grade 4 CRS

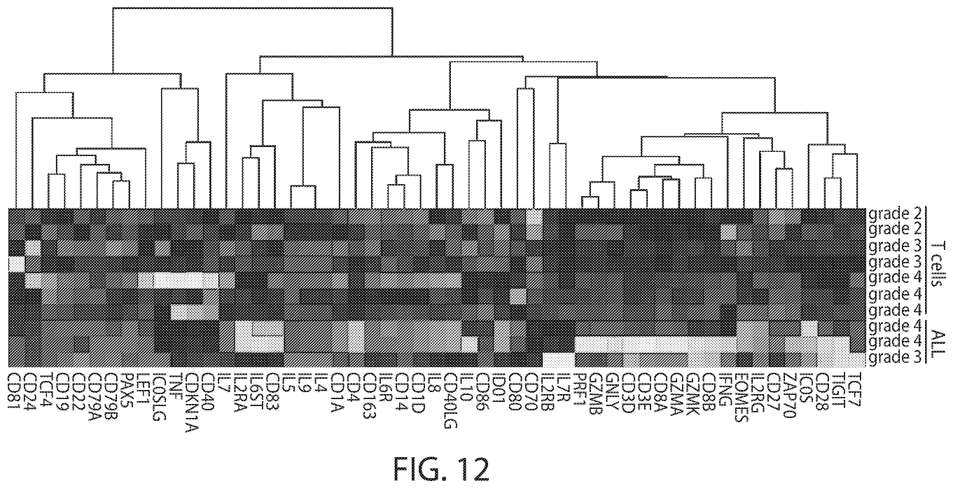

Cytokine analysis of patients who have received CD19 CAR T cell therapy for leukemia demonstrated that while many cytokines are elevated during CRS, only a few contribute to a predictive model of which patients will go on to develop grade 4 CRS after T cell infusion (Teachey Cancer Discovery 2016). Peripheral blood and isolated mononuclear cells (PBMCs) were collected from patients who had received CD19 CAR T cell therapy for treatment of ALL as part of a phase I clinical trial on their first day of fever after T cell infusion. Seven of ten patient samples had detectable peripheral CAR T cells, and of these patients, three experienced grade 2 CRS, one grade 3, and three grade 4. The remaining three samples had no detectable peripheral T cells, but only circulating ALL cells; of these patients, two were classified as grade 4 CRS and one grade 3. Unsupervised clustering analysis was performed on these samples; distinct transcriptional profiles were determined for grade 2-3 and grade 4 CRS (FIG. 12). T cells from patients that developed grade 4 CRS had elevations in granzyme B, perform, IFN-γ, Zap70, EOMES and Lag-3 transcripts, and suppressed levels of tumor necrosis factor-α, IL-1β and CCR7 as compared to those with grade 2-3 CRS. B cell transcripts, such as CD79, Pax5 and CD19, were only elevated in the three samples with circulating leukemia.

### CD19 CAR T Cells Did not Produce IL-6 in Patients Experiencing CRS

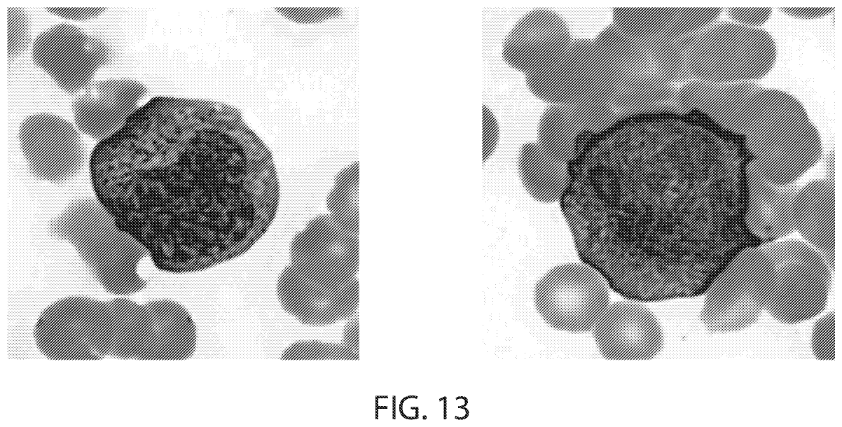

Having demonstrated that CAR T cells did not produce IL-6 in vitro, experiments were performed to confirm this finding in a relevant clinical context. Transcriptional analysis of patients experiencing fever who went on to develop CRS revealed that none of the samples containing CAR T cells demonstrated detectable levels of IL-6 transcript, with all IL-6 transcript levels measuring below the lower-limit of detection (<1 copy of RNA transcript per cell, FIG. 12). Similarly, no samples containing only leukemia cells had detectable levels of IL-6, confirming that in these patients neither the T cells nor leukemia were responsible for IL-6 production. Examination of the T cells from this collection using light microscopy demonstrated a highly activated phenotype, with large, irregular nuclei, open chromatin and irregular plasma membranes (FIG. 13).

### Discussion

There has been a lack of mechanistic understanding of CRS, e.g., CRS associated with CAR T cell therapiesThe results herein provide biological insight into the source of IL-6 and the role it has in CAR T cell activity. In particular, the results herein demonstrated that monocyte-lineage APCs produced IL-6 in response to CAR-mediated T cell recognition of target leukemia, and that T cell transcriptional and cytotoxic activity were not affected by the presence of IL-6.

These findings demonstrated that monocyte-derived immature dendritic cells yielded the greatest IL-6 signal in response to CAR-mediated T-cell activation. The identification of the cellular source of IL-6, along with the confirmation that CD19 CAR T cells from patients did not produce IL-6, highlights a central physiology of this syndrome. The results herein included observation of higher levels of IL-6 when APCs were combined with targets in the presence or absence of un-activated T cells when T cells and APCs were separated in the trans-well setting. One explanation could be that direct cell:cell contact between targets and APCs may inhibit secretion of IL-6, via inhibitory signaling based on cell-surface co-receptors. Alternatively, the micropore material of the trans-well insert may provide non-specific stimulation to the APCs that is not delivered by the inert plastic of the traditional plate well, and that this stimulation may result in enhanced IL-6 secretion. The overall pattern, however, remained the same, with the only statistically-relevant increase in IL-6 secretion resulting from the combination of CD19 CAR T cells, targets and APCs, suggesting that IL-6 secretion by APCs is stimulated not by cell:cell contact, but instead by a soluble factor present when CAR T cells kill targets.

Transcriptional mapping using the trans-well system allowed for identification of the cellular sources of all cytokines evaluated. IFN-γ was produced by T cells only, consistent with the findings from cytokine quantitation presented in FIG. 8. IL-2, GMCSF and IL-8 were produced by both cell populations, albeit with clear predominance of APCs or T cells for each molecule. Low levels of IL-2 were derived from APCs, with the predominance coming from T cells. Examination of secretion patterns from FIG. 8 demonstrated that when CAR T cell were combined with targets, IL-2 levels were ˜40000 pg/mL at 48 hours, similar to that seen with CAR T cells and targets were combined with monocytes and immature dendritic cells. The presence of mature dendritic cells and macrophages, however, resulted in significantly greater IL-2 levels, nearing 160000 pg/mL, a 4-fold increase. While these differences in concentration were not entirely correlated to the differences in transcription, it is possible that the monocyte lineages responsible for IL-2 production were mature dendritic cells and macrophages. Several possibilities may explain the significant differences in cytokine quantity not explained by transcriptional differences. Altered receptor expression or recycling on the part of the APCs may cause a fluctuation in the soluble IL-2 present at time of collection. Alternatively, APCs may secrete other soluble factors that enhance stability or lower consumption of IL-2. GMCSF demonstrated a nearly identical pattern, with increased secretion when activated T cells were combined with APCs, and transcriptional evidence of two cellular sources. In this case, immature dendritic cells appeared to be the source of APC-derived GMCSF, with mature DCs and macrophages also contributing. Detection of IL-8 at the protein level only occurred when activated T cells were combined with APCs, with very low levels detected when T cells were alone or with targets, while transcriptional analysis demonstrated two cellular sources. APC IL-8 transcript levels were ˜4_{log} greater than T cell transcript levels, which may explain these dynamics. IL-2 and GMCSF transcripts both demonstrated a ˜2_{log} discrepancy. Finally, IL-8 was the only cytokine that demonstrated significantly greater levels at the 18 hour time point, with all others peaking at 48 hours, suggesting that IL-8 may be an early component of the CRS cascade.

Maude et al. described the vast majority of patients with ALL reported to have developed cytokine release syndrome, with 27% experiencing severe CRS requiring tocilizumab (Maude NEJM 2015). Anti-IL-6R therapy has been effective in managing this toxicity, and in the majority of cases has led to a rapid clinical improvement. The patient whose cells were used in the study in this Example experienced a rapid improvement of an ongoing respiratory and hemodynamic insufficiency. The decision to invoke anti-IL-6 therapy has been at the discretion of the clinical trial team, as the effect of disrupting IL-6 signaling on CAR T cell activity has remained unknown. These findings herein demonstrate that monocyte-lineage derived IL-6 does not alter CAR T cell transcriptional signatures, and that this transcriptional stability corresponds to stability of cytotoxic function. The results herein suggest that APCs and APC-produced IL-6 are not necessary for CAR T cell activity in vivo, and are likely bystanders that do not play a role in target killing in vivo. These findings suggest that abrogation of IL-6 signaling after CAR T cell infusion should have no impact on anti-tumor response. Conclusion

Management of CRS following CD19 CAR T cell therapy has largely been empiric given the limited biological understanding of this syndrome. The results herein demonstrate that CAR T cells do not produce IL-6 (rather, they are produced by APCs), and that the presence of IL-6 does not alter T cell transcriptional activity or cytotoxicity. These results allow for more informed use of anti-IL-6 therapies to control the significant morbidity associated with toxicity from CRS while preserving the efficacy of the CAR T cell therapy. The data herein can support the blocking of IL-6 before symptoms of CRS appear without changing CART19 efficacy. Based on the data herein, a clinical trial has been designed to allow for early administration of tocilizumab following CD19 CAR T cell therapy. The clinical trial is described in greater detail in Example 4 below. The early administration of tocilizumab (e.g., before or soon after CRS symptoms occur) may significantly reduce the incidence of CRS toxicity while maintaining robust anti-tumor efficacy.

### Example 4: A Phase 2, Two Cohort Study of the Tocilizumab Optimization Timing for CART19 (CTL019) Associated Cytokine Release Syndrome (CRS) Management in Pediatric Patients with CD19 Expressing Relapsed/Refractory B-Cell Acute Lymphoblastic Leukemia (ALL)

Clinical Experience with Tocilizumab

Toxicities such as CRS and macrophage activation syndrome (MAS) have been observed in CART19 patients (162 patients receiving the product in 7 studies that include adult and pediatric and lymphomas as of May 2015). CRS has been the most significant SAE seen in adult and pediatric patients treated with CTL019. CRS typically begins within 2 weeks of CART19 infusion and it starts with several days of fevers. In all cases, evaluation for infections is done. Fevers tend to be spiking and can be associated with rigors, anorexia, nausea, diarrhea, diaphoresis, capillary leak, hypoxia and hypotension. In 25-30% of case ICU level care, ventilator support and pressors have been needed. Observations have noted highly elevated IL-6 concentrations during CRS. In addition, the reaction typically appears to be associated with MAS. This can be manifest by evidence of elevated ferritin, but can also be associated with hypofibrinogenemia, cytopenias, altered mental status, and other complications.

Tocilizumab is an anti-IL6 receptor antibody and has been administered at a dose of 8-12 mg/kg on CHP959. In many cases, CRS has been severe but reversible. However there have been several cases of refractory CRS that resulted in death in adult patients, generally complicated by resistant infections at the same time. The risk of CRS is highly significantly related to tumor burden, so that treating patients with less tumor burden may result in less severe cytokine release syndrome. However, additional contributory patient and CART19-related factors cannot be ruled out.

Since CRS mechanistically is a required part of the antitumor mechanism of in vivo CART19 cell expansion and tumor killing, tocilizumab has been administered for CRS with worsening respiratory distress, including hemodynamic instability despite intravenous fluids and moderate vasopressor support, rapid clinical deterioration, pulmonary infiltrates, increasing oxygen requirement including high-flow oxygen and/or need for mechanical ventilation.

CRS/MAS were successfully managed in the majority of patients with supportive care when this toxicity was mild or moderate and with anti-cytokine therapy like tocilizumab plus supportive care. Severe CRS/MAS has responded rapidly (often in hours) to the administration of tocilizumab as needed in all CLL, NHL and pediatric ALL patients treated to date.

In pediatric patients with ALL treated under CHP959 and the Novartis-UPenn multisite trial B2205J, CRS of grades 1-4 was reported for 62 (89.8%) patients. 21 of 62 patients (33.8%) required anticytokine therapy. CRS was reversible in all patients but one patient with administration of 1 to 3 doses of tocilizumab. In adult patients with ALL treated under in the Penn study B2102J CRS was reported in all patients. Fifty percent of these patients required anti-cytokine therapy (one or two tocilizumab doses), which led to complete resolution of CRS.

### Impact of Tocilizumab on CART19 Expansion

Graphical exploration of CART19 cellular kinetics in 25 pediatric ALL patients (CHP959, B2205J, and B2202) did not suggest an impact of tocilizumab on CART19 expansion. In two examples of pediatric ALL patients from a clinical study, CHP959 (a phase I clinical study of pediatric ALL patients given CART19), sampled qPCR assessments showed that the rate of expansion of CART19 cells appeared similar before and after administration of the first dose of tocilizumab when administered per the criteria of the CRS treatment algorithm described herein.

In preliminary analyses to date (n=25 pediatric ALL patients), no discernable impact of tocilizumab on the rate of expansion based upon a nonlinear mixed effect model has been detectable.

### Impact of Tocilizumab on Anti-Tumor Activity

In patients treated in CHP959, 100% of patients that were treated with tocilizumab for grade 4 CRS subsequently went into remission. Patients treated with tocilizumab achieving a CR/CRi tended to have a 2-fold higher T-cell exposure (AUC28d) compared with patients that were not treated with tocilizumab (data not presented), however, this did not impact the clinical response. Additional factors characterized patients that received tocilizumab, including greater severity of CRS, which in turn correlated with higher tumor burden just prior to CART19 cell infusion. Within the CR/CRi responses subgroup (n=46) of CHP959, fewer patients (n=15) received tocilizumab than did not receive (n=31). Another on-target effect that was seen only in responding patients was the depletion of normal CD19+ B cells. See, e.g., Grupp et al. N. Engl. J. med. 2013; and Porter et al. N. Engl. J. Med 2011; 365(8):725-33. Preliminary comparison of the duration of remission (DOR) of patients who received tocilizumab versus those who did not receive tocilizumab suggests no impact on CART19 tumor efficacy when administered via the standard CRS treatment algorithm, e.g., treatment algorithm described herein.

### Summary of the Phase 2 Study

This example describes a phase 2, two cohort, open-label study to describe the efficacy of administration timing of tocilizumab on CART19 (CTL019) associated CRS safety events in pediatric patients with CD19 expressing relapsed and refractory B-cell acute lymphoblastic leukemia with high versus low pre-infusion tumor burden following redirected autologous T cells transduced with the anti-CD19 lentiviral vector (CART19/CTL019).

The primary objective of this study is to describe the frequency of grade 4 CRS. The secondary objectives are:

1. To describe the tumor response as assessed by Day 28 rate of CR with MRD negative bone marrow and by the duration of remission
2. To describe the CART19 (CTL019) cellular kinetics; and
3. To describe additional safety endpoints. 3
The exploratory objectives are:
1. To compare rate of CART19 (CTL019) expansion before and after first tocilizumab dose; and
2. To describe the profile of soluble immune factors that may be key to cytokine release syndrome.

Inclusion criteria are designed to include pediatric patients aged 1-24 years with CD19 expressing relapsed/refractory B-cell acute lymphoblastic leukemia (ALL).

The study product is CART-19 cells transduced with a lentiviral vector to express either anti-CD19ζ scFv TCζ:41BB, administered by i.v. injection using an intra-patient dose escalation approach: 10% on day 0, 30% on day 1 with a total dose goal of ˜1.5×10⁷-5×10⁹ (˜0.3×10⁶-1.0×10⁸/kg) T cells.

Two cohorts are defined based upon pre-infusion high versus low tumor burden; with the high tumor burden cohort to receive protocol defined early anti-cytokine intervention (i.e. tocilizumab) for CRS management and the low tumor burden cohort to receive standard anti-cytokine intervention (i.e. tocilizumab) for CRS.

The duration of CART-19 administration will be based on the total volume to be infused and the recommended infusion rate of 10-20 mL per minute. The transduced T cells will be administered by slow IV push. T cells are expected to persist at detectable levels in circulation for months or longer in many patients.

### Dose and Treatment Regimen

A dose of 1.5×10⁷-5×10⁹ cells or 0.3×10⁶-1.0×10⁸/kg of CART19 cells will be used. Because there are about 1×10¹² T cells in a healthy adult (equivalent to 2×10¹⁰ T cells/kg), the proposed total (100%) dose is equivalent to about 0.5% of the total body mass of T cells. Therefore, the initial frequency of cells to be present should be at about 0.5% at baseline following infusion. As an additional safety feature, the cells will be administered using a split dosing approach as described in the “CART19 transduced T cells administration” section below.

### CART19 Transduced T Cells Administration

CART19 T cells will be given, up to a total dose of 1.5×10⁷-5×10⁹ (0.3×10⁶-1.0×10⁸/kg) total cells. The actual number of transduced CART19 cells administered will depend upon transduction efficiency. The following schedule will be used:

Day 0: “10%”-1.0×10⁷/kg

Day 1: “30%”-3.0×10⁷/kg if patient is clinically stable from prior day's infusion

If the target dose is not achieved in manufacturing, a product that meets all release criteria may be infused. The toxicities that would preclude the next dose of T cells are fever or clinical instability. Toxicities attributable to the prior chemotherapy, such as cytopenias, will not impact infusion in a stable patient.

Timing and Doses of Subsequent CART-19 Infusions

For patients who have had i) evidence of brief B cell aplasia with subsequent B cell recovery (suggesting rapid CAR clearance), or ii) fever and other reversible toxicities without evidence of CAR expansion/LGLs or response, or iii) no response, or a partial or temporary response to the initial infusion, it may be that the initial dose of cells was not adequate to produce a full therapeutic effect, or the cells may not have persisted long enough to produce longer-term disease control. In these cases, it may be appropriate to give more CART-19 cells (subsequent infusions). Subsequent infusions will be given no earlier than Day 14.

It is possible that a subject's cumulative dose could exceed 100% of the dose specified above. More than 100% of the cell dose may be given in aliquots to sustain an initial response or address rapid CAR clearance (as evidenced by e.g. B cell recovery) if the cells grew well and a sufficient number are available. In this scenario, additional doses of 30% (if available) would be given at 2 week+ intervals. The rationale for this dosing regimen is that there does not appear to be a significant dose-response relationship with the initial dose. We have observed varying and significant degrees of cell expansion post infusion, making the amount of the infused cells less relevant. Thus, the multiple doses given over time may be more efficacious in sustaining a response. In terms of safety, the worst toxicities have been observed with the first infusion. There has been a minimal toxicity with subsequent infusions in the few patients that have received them. Therefore, we consider the potential benefits of administering a greater cell dose over time to outweigh the potential risks. However, the cumulative dose will not exceed 1.5×10⁸/kg administered over time in several doses.

### Study Design

The study will have three sequential phases: 1) a screening phase, 2) a manufacturing and pre-treatment phase, including apheresis (if applicable) and chemotherapy (if applicable), and 3) a treatment phase, including a CART19 transfused cell infusion and follow up evaluations.

Once patient eligibility is confirmed, patients who do not have apheresis product suitable for manufacturing will have cells collected by leukapheresis to obtain peripheral blood mononuclear cells (PBMC) for this purpose. Cells will be transduced with the anti-CD19 TCζ/4-1BB lentiviral vector, expanded in vitro and then frozen for future administration. Cryopreserved historical apheresis products collected from the patient prior to study entry are usable for CART19 manufacturing if collected at an appropriately certified apheresis center and the product meets adequate mononuclear cell yields. If a historical apheresis product is not available, an apheresis procedure will be scheduled for cell procurement after study entry.

Unless contraindicated and medically not advisable based on previous chemotherapy, patients will be given conditioning chemotherapy prior to CART19 cell infusion with the intent of lymphodepletion. Additionally, if the patient's white blood cell (WBC) count is ≤1,000/uL, conditioning/lymphodepleting chemotherapy is not required. The chemotherapy will be planned so that the last dose is completed 1-4 days before the planned infusion of CART19 cells. The chemotherapy start date will vary based on the duration of the selected chemotherapy regimen. If the period from chemotherapy to CART19 infusion is delayed for 4 weeks or more, the patient will need to be re-treated with lymphodepleting chemotherapy prior to CART19 infusion.

Two study cohorts are planned as defined by tumor burden (TB) (defined by the highest blast percentage measured by differential of a bone marrow aspirate or biopsy or multiparameter flow cytometry for MRD) at the timepoint just preceding CART19 infusion:

1. Cohort A: Patients with ≥40% blasts in the bone marrow at pre-infusion (˜Day −5 to −1) will be enrolled in the early tocilizumab cohort and will follow early CRS treatment algorithm.

2. Cohort B: Patients with <40% blasts in the bone marrow at pre-infusion (˜Day −5 to −1) will follow the standard CRS Rx algorithm.

### Patient Inclusion Criteria

Patient inclusion criteria includes the following:

1. Relapsed or refractory B-cell ALL:
a. 2nd or greater relapse (marrow or CNS) OR
b. Any relapse after allogeneic HSCT and ≥6 months from SCT at infusion OR
c. Any relapse after CAR-modified T cell therapy OR
d. Refractory disease defined as having not achieved an MRD-negative CR after >2 chemotherapy regimens/cycles (1 cycle for relapsed patients) OR
e. Patients with Ph+ ALL are eligible if they are intolerant to or have failed tyrosine kinase inhibitor therapy OR
f. Ineligible for allogeneic SCT because of: i. Comorbid disease
   ii. Other contraindications to allogeneic SCT conditioning regimen
   iii. Lack of suitable donor
   iv. Prior SCT
   v. Declines allogeneic SCT as the therapeutic option after documented discussion, with expected outcomes, about the role of SCT with a BMT physician not part of the study team
g. Patients with CNS3 disease will be eligible if CNS disease is responsive to therapy (at infusion, must meet criteria in Section 5.2)
   2. Documentation of CD19 tumor expression in bone marrow or peripheral blood by flow cytometry at relapse (or a recent marrow in the case of refractory disease). If the patient has received CD19-directed therapy (i.e. blinatumomab), then the marrow should be obtained after this therapy to show CD19 expression.
   3. Adequate organ function defined as:
a. A serum creatinine based on age/gender as follows:

TABLE 21A
Serum creatinine based on age and gender
Maximum Serum Creatinine (mg/dL)

| Age | Male | Female |
|---|---|---|
| 1 to <2 years | 0.6 | 0.6 |
| 2 to <6 years | 0.8 | 0.8 |
| 6 to <10 years | 1.0 | 1.0 |
| 10 to <13 years | 1.2 | 1.2 |
| 13 to <16 years | 1.5 | 1.4 |
| ≥16 years | 1.7 | 1.4 |

b. ALT<500 U/L
c. Bilirubin<2.0 mg/dl d. Must have a minimum level of pulmonary reserve defined as ≤Grade 1 dyspnea, pulse oximetry>92% on room air; DLCO>40% (corrected for anemia) if PFTs are clinically appropriate as determined by the treating investigator e. Left Ventricle Shortening Fraction (LVSF)≥28% or Ejection Fraction (LVEF)≥40% confirmed by ECHO, or adequate ventricular function documented by a scan or a cardiologist.
   5. Evidence of disease by standard morphologic or MRD criteria. A clinical marrow showing disease may be performed at enrollment or within 12 weeks of enrollment.
   6. Age 1-24 years.
   7. Adequate performance status (Lansky or Karnofsky score ≥50).
   8. Subjects of reproductive potential must agree to use acceptable birth control methods.

### CART19 Product Infusions

The transduced T cells will be administered by slow IV push. No more than 10 mL/kg of total volume will be delivered to the patient. The duration of CART-19 administration will be based on the total volume to be infused and the recommended infusion rate of 10-20 mL per minute.

Vital signs (temperature, respiration rate, pulse, blood pressure, and oxygen saturation as clinically indicated) will be measured within 10 minutes prior to the infusion, within 10 minutes after the infusion, and then every 15 minutes for at least one hour. If the subject's vital signs are not satisfactory and stable one hour post-CART19 infusion, vital signs will continue to be monitored every hour or as clinically indicated. The subject will be discharged after the physician managing their care on the day of each infusion has determined that they are in satisfactory condition.

### Febrile Reaction

In the event of febrile reaction, an evaluation for infection should be initiated, and patients managed appropriately with antibiotics, fluids and other supportive care as medically indicated and determined by the treating physician. In the event that the patient develops sepsis or systemic bacteremia following the CAR T cell infusion, appropriate cultures and medical management should be initiated. If a contaminated CART19 T cell product is suspected, the product can be retested for sterility using archived samples that are stored in the CVPF. Consideration of CRS as the most likely etiology should be given.

### Assessment of Laboratory Parameters for CRS

Hematology, coagulation and chemistry safety assessments will be performed at study visits. Side effects following CART19 cell infusions can induce high fevers and should be expected. If high fevers (≥101.5° F./38.6° C.) occur following CART19 infusion, it is recommended that ferritin, LDH and CRP levels be monitored QD until resolution of the fever (below 101.5° F./38.6° C.). Other chemistry parameters should be monitored or as clinically indicated if CRS is suspected.

### Cytoreductive/Lymphodepleting Chemotherapy

Prior to CART19 cell infusion, an additional chemotherapy cycle is planned. While the choice of chemotherapy will be at the investigator's discretion depending on the patient's underlying disease and prior therapies, fludarabine (30 mg/m2/day×4 days) and cyclophosphamide (500 mg/m2/day×2 days) are the preferred agents, as there is the most experience with the use of these agents in facilitating adoptive immunotherapy on the ongoing pediatric murine CART19 study CHP959.

The lymphodepleting chemotherapy prior to CART19 cell infusion is not required if patient's WBC≥1,000/uL. Additionally, if the period between chemotherapy and CART19 infusion is delayed 4 or more weeks, the patient may need to be re-treated with lymphodepleting chemotherapy prior to CART19 infusion.

The chemotherapy will be planned so that the last dose is completed 1-4 days before the planned infusion of CART19 cells for ALL. Each regimen is of different duration so the start day of chemotherapy will vary. The purpose of the chemotherapy is to induce lymphopenia in order to facilitate the engraftment and homeostatic expansion of CART19 cells. In addition, chemotherapy is intended to control the ALL. The chemotherapy is not investigational and may be given by a patient's local oncologist within the specified time frame.

### CART19 (CTL019) Infusions

Subject infusions are to begin 1 to 4 days after completion of chemotherapy as indicated in the “Cytoreductive/Lymphodepleting chemotherapy” section herein.

Subjects will undergo tests and procedures in accordance with a Visit Evaluation Schedule. This includes a CBC with differential prior to each infusion, as well as an assessment of CD3, CD4 and CD8 counts prior to the 1st infusion since chemotherapy is given in part to induce lymphopenia. CART19 cells will be infused as described above in the Dose/Treatment Regimen section.

### Tumor Burden Based CRS Management

Two study cohorts are planned as defined by tumor burden at the time just preceding CART19 infusion:

a) Cohort A: Patients with ≥40% blasts in the bone marrow at pre-infusion (˜Day −5 to −1) will be enrolled in the early anti-cytokine cohort and will follow early CRS management algorithm.

b) Cohort B: Patients with <40% blasts in the bone marrow at pre-infusion (˜Day −5 to −1) will follow the standard CRS management algorithm.

### Efficacy Assessments

Tumor response assessments will be done at baseline (prior to CART19 infusion) and then at Day 28 and Months 3, 6, 9 and 12 after CART19 cell infusions or until the patient requires alternative therapy for their disease. Assessments will be made as clinically indicated by physical exam, chest x-ray (if clinically indicated), CSF evaluation, hematology blood panel, and bone marrow biopsy and aspirate.

Disease assessment collection plan is detailed in Table 21B.

TABLE 21B
Disease Assessment Collection Plan
(assessments are all standard of care)

| | Pre-Infusion | Post Infusion |
|---|---|---|
| Procedure | Assessments | Assessments |
| Bone marrow aspirate and | Mandated | Day 28, Months 3, 6, 9 and |
| biopsy for blast cell counts | within 12 | 12 |
| | weeks of | |
| | enrollment | |
| Peripheral Blood for blast, | Mandated | Day 28, Months 3, 6, 9 and |
| neutrophil and platelet cell | | 12 |
| counts | | |
| CSF Assessment for CNS | Mandated | Day 28, Months 3, 6, 9 and |
| disease | | 12 |
| Physical Exam for | Mandated | If clinically indicated |
| extramedullary disease | | |
| MRD assessment of bone | Mandated | Day 28, Months 3, 6, 9 and |
| marrow by flow cytometry | | 12 |
| (every patient) | | |
| BCR-ABL assay of blood | Mandated | Day 28, Months 3, 6, 9 and |
| and bone marrow aspirate | | 12 |
| for patients with Ph+ ALL | | |

### Physical Exam

A physical examination will be used to assess evidence of extramedullary disease in the liver, spleen, lymph node, skin, gum infiltration, testicular involvement and other sites if applicable. Extramedullary involvement is to be assessed at screening and will be followed as clinically appropriate.

### Bone Marrow Aspirate/Biopsy and Peripheral Blood

Bone marrow biopsies and aspirate will be measured for tumor evaluations and efficacy analysis.

### Cerebrospinal Fluid (CSF) Assessment

If CNS symptoms are present at Screening/Enrollment, a lumbar puncture will be performed to assess CNS leukemic involvement. CSF will be assessed at baseline (Day −1) and on Day 28. CSF will be analyzed for cell count and differential, cytology, and for the presence of CART19 cells. Additionally, CSF may be assessed as clinically indicated during the height of cytokine release syndrome (CRS).

### Extramedullary Disease

If extramedullary disease is present prior to treatment, this will be followed as clinically appropriate.

### Minimal Residual Disease (MRD)

All patients will have multiparameter flow cytometry on bone marrow aspirate for MRD status at each time point a bone marrow aspirate is performed.

### Quantitative BCR-ABL: Ph+ ALL Patients

Bone marrow aspirates sampled at the time points for tumor assessments will additionally be analyzed for quantitative BCR-ABL levels for Ph positive ALL patients only.

### ALL Response Criteria

The response criteria will be evaluated accordingly to the Table 21C. The definitions are primarily based on the standardized response criteria defined by National Comprehensive Cancer Network (NCCN) Guidelines (NCCN, 2013 v.1) and further supported by the workshop report from American Society of Hematology (ASH) and the International Working Group (IWG) guideline for acute myeloid leukemia (AML). The Cheson IWG guideline and Appelbaum ASH report were used in recent drug approvals (e.g. Marqibo) in ALL, prior to the NCCN guideline availability. The NCCN guidance is a more recently published updated US based guideline for ALL.

Efficacy assessments will be performed based on bone marrow and blood morphologic criteria, physical examination findings, along with laboratory assessments of CSF and bone MRD assessment. The overall disease response is determined at a given evaluation using the criteria described in Table 21C.

TABLE 21C
Overall disease response classification at a given evaluation time

| Response category | Definition |
|---|---|
| Complete remission (CR) | All the following criteria are met: |
| | Bone marrow |
| | Trilineage Hematopoiesis (TLH) and <5% blasts |
| | Peripheral blood |
| | Neutrophils >1.0 × 109/L, and |
| | Platelets >100 × 109/L, and |
| | Circulating blasts <1% |
| | Extramedullary disease |
| | No evidence of extramedullary disease (no CNS disease, |
| | mediastinal disease CR, no other extramedullary sites |
| | involvement) |
| | Transfusion independency |
| | No platelet and/or neutrophil transfusions within 1 week |
| | before peripheral blood sample for disease assessment |
| Complete remission with | All criteria for CR as defined above are met, except that |
| incomplete blood count | the following exist: Neutrophils ≤1.0 × 109/L, or |
| recovery (CRi) | Platelets ≤100 × 109/L, or |
| | Platelet and/or neutrophil transfusions within week |
| | before peripheral blood sample for disease assessment |
| Complete remission (CR) | All criteria for CR or CRi as defined above are met, |
| with residual mediastinal | except that mediastinal disease as defined by CRu or PR |
| disease | is observed: |
| No response (Treatment | Failure to attain the criteria needed for any response |
| failure) | categories |
| Relapsed Disease | Only in patients with a CR or CRi: |
| | Reappearance of blasts in the blood (≥1%), or |
| | Reappearance of blasts in bone marrow (≥5%), or |
| | (Re-)appearance of any extramedullary disease after CR |
| Unknown | In case the response assessment was not done, the |
| | baseline assessment was not done, the assessment was |
| | incomplete or was not done within the respective time |
| | frame. If there is evidence of relapse, the overall |
| | response will assessed as relapse with the relapsed |
| | component alone. |

The NCCN guideline has defined mediastinal response criteria including CRu and PR. In the case a patient achieves CR or CRi at all other non-mediastinal disease sites, and has residual mediastinal disease (CRu or PR), a category for overall disease response of CR or CRi with residual mediastinal disease has been included in this document, which is not part of the NCCN guidance.
The NCCN guidance has defined a progressive disease (PD) category. In this document, PD is considered the same as “No response” or “Treatment failure”, which is consistent with the Cheson et al. (2003) guideline. The difference between PD and “No response” in ALL is not believed to be clinical meaningful.

### Recording of Adverse Events

Adverse events will be recorded.

### Grading System of Cytokine Release Syndrome

Recipients of CART19 cells may develop CRS. Data from a small number of patients shows marked elevations in IL6, IFN-g, and less intensely TNF. Elevations in clinically available markers of inflammation including ferritin and CRP have also been observed to correlate with the clinical CRS syndrome.

Symptoms usually occur 1-14 days after cell infusion, but the syndrome is not defined by the timing of the reaction. Patients developing any symptoms attributable by the investigator as related to cytokine release should be reported has having a CRS. Symptoms may include, e.g., high fevers, rigors, nausea, vomiting, anorexia, fatigue, headache, myalgia/arthralgia, hypotension, dyspnea, tachypnea, hypoxia, altered mental status, end organ dysfunction, and signs of MAS including elevated ferritin.

For the purposes of reporting and grading on clinical trials using CART19 cells, we will use the following grading for CRS Toxicity. The start date of CRS is a retrospective assessment of the date of onset of persistent fevers and/or myalgia consistent with CRS and not explained by other events (i.e. sepsis). The stop date of CRS is defined as the date when the patient has been afebrile for 24 hours and off vasopressors for 24 hours. Afebrile is defined as a temperature<38.0° C. (100.4° F.).

TABLE 21D
CRS grading criteria
CRS Toxicity Grade (Modified)

| 1 | 2 | 3 | 4 | 5 |
|---|---|---|---|---|
| Mild | Moderate | More severe reaction: | Life-threatening | Death |
| reaction: | reaction | Hospitalization required for | complications such | |
| Treated with | requiring IV | management of symptoms | as hypotension | |
| supportive | fluids or | related to organ dysfunction | requiring high dose | |
| care such as | parenteral | including grade 4 LFTs or | pressors (see Table | |
| anti-pyretics, | nutrition; some | grade 3 creatinine related to | E), or hypoxia | |
| anti-emetics | signs of organ | CRS and not attributable to | requiring | |
| | dysfunction | any other conditions. This | mechanical | |
| | (i.e. grade 2 | excludes management of fever | ventilation | |
| | creatinine or | or myalgias. Includes | | |
| | grade 3 liver | hypotension treated with | | |
| | function tests | IVFs* or low-dose pressors, | | |
| | [LFTs] related | coagulopathy requiring fresh | | |
| | to CRS and not | frozen plasma (FFP) or | | |
| | attributable to | cryoprecipitate, and hypoxia | | |
| | any other | requiring supplemental | | |
| | condition). | oxygen (nasal cannula | | |
| | Hospitalization | oxygen, high flow oxygen, | | |
| | for | Continuous Positive Airway | | |
| | management of | Pressure [CPAP] or Bilateral | | |
| | CRS related | Positive Airway Pressure | | |
| | symptoms | [BiPIP]. Patients admitted for | | |
| | including | management of suspected | | |
| | fevers with | infection due to fevers and/or | | |
| | associated | neutropenia may have grade 2 | | |
| | neutropenia. | CRS. | | |

*CRS Grade 3 language clarification: “hypotension treated with intravenous fluids” is further defined as hypotension requiring multiple fluid boluses for blood pressure support.

TABLE 21E
High Dose Vasopressor Use Definition of
“High-Dose” Vasopressors

| | |
|---|---|
| Vasopressor | Dose for ≥3 hours |
| Norepinephrine monotherapy | ≥0.2 mcg/kg/min |
| Dopamine monotherapy | ≥10 mcg/kg/min |
| Phenylephrine monotherapy | ≥200 mcg/min |
| Epinephrine monotherapy | ≥0.1 mcg/kg/min |
| If on vasopressin | High-dose if vaso + Norepinephrine |
| | Equivalent (NE) of ≥0.1 mcg/min |
| | (using Vasopressin and Septic Shock |
| | Trial (VASST) formula) |
| If on combination vasopressors | Norepinephrine equivalent of ≥20 |
| (not vasopressin) | mcg/min (using VASST formula) |

Vasopressin and Septic Shock Trial (VASST) Equivalent Equation: Norepinephrine equivalent dose = [norepinephrine (mcg/min)] + [dopamine (mcg/kg/min) ÷ 2] + [epinephrine (mcg/min)] + [phenylephrine (mcg/min) ÷ 10]
Criteria from Russell et al, 2008.
Note:
Pediatric weight adjustments should be taken into consideration.

### Toxicity Management

### Febrile Reaction

In the event of febrile reaction, an evaluation for infection should be initiated, and patients managed appropriately with antibiotics, fluids and other supportive care as medically indicated and determined by the treating physician. In the event that the patient develops sepsis or systemic bacteremia following CAR T cell infusion, appropriate cultures and medical management should be initiated. If a contaminated CART19 T cell product is suspected, the product can be retested for sterility using archived samples that are stored in the CVPF. Consideration of CRS (see below) should be given.

### Cytokine Release Syndrome (CRS)/Macrophage Activation Syndrome (MAS)

High Tumor Burden Cohort (early tocilizumab)—patients with ≥40% blasts in bone marrow at pre-infusion (˜Day −5 to Day −1): Intervention with tocilizumab (8-12 mg/kg) will occur when 2 temperatures>38.5° C. occur within a 24 hour period measured at least 4 hours apart. If and when the patient experiences clinical CRS, the standard CRS treatment approach will then be used.

Low Tumor Burden Cohort—patients with <40% blasts in bone marrow at pre-infusion (˜Day −5 to Day −1), with clinical CRS will follow the CRS treatment algorithm outlined in Table 21F.

Tocilizumab should be used as a single, weight-based dose of 8-12 mg/kg at the time of hemodynamic instability. This management approach is designed to avoid life-threatening toxicities, so the timing of the tocilizumab should be individualized, in close consultation with the study team. Steroids have not always been effective in this setting and may not be necessary given the rapid response to tocilizumab. Because steroids will interfere with CART19 function and efficacy, if used, they should be rapidly tapered.

Upon developing the prodrome of high-persistent fevers following CART19 infusion, patients should then be followed closely. Infection and tumor lysis syndrome work up should be immediately undertaken. The pharmacy should be notified of the potential need for tocilizumab. Patient management in an intensive care unit may be required and the timing is dependent upon local institutional practice. In addition to supportive care, tocilizumab may be administered in cases of moderate to severe CRS, especially if the patient exhibits any of the following:
Hemodynamic instability despite intravenous fluid challenges and moderate stable vasopressor support
Worsening respiratory distress, including pulmonary infiltrates, increasing oxygen requirement including high-flow 02, and/or need for mechanical ventilation.
Any other signs or symptoms of rapid deterioration despite medical management

Not all Grade 4 CRS reactions following CART19 have been immediately treated with tocilizumab and decisions are, in part, based upon the rapidity of the syndrome onset and underlying patient reserve.

CRS has been associated with biochemical and physiologic abnormalities consistent with MAS. Moderate to extreme elevations in serum C-reactive protein (CRP) and ferritin have been seen with

CART19 associated CRS, however the magnitude and kinetics vary greatly between individual patients. CRS management decisions should be based upon clinical signs and symptoms and response to interventions, not these laboratory values per se.

CTCAE grading of CRS relates to its occurrence with acute infusional toxicities, whereas the CRS associated with CART19 therapy is not acute, but rather delayed.

TABLE 21F
CRS Treatment Algorithm

Pretreatment
Acetaminophen/paracetamol and diphenhydramine/H1 anti-histamine
Prophylaxis for complications of TLS as appropriate
CART19 infusion
Prodromal syndrome: low grade fevers, fatigue, anorexia (hours to days)
Observation, rule out infection (surveillance cultures)
Antibiotics per local guidelines (febrile neutropenia)
Symptomatic support
Symptom progression: High fevers, hypoxia, mild hypotension
1st Line Management:
Oxygen, fluids, low dose vasopressor support, antipyretics
Monitor/manage complications of TLS
Further symptom progression:
Hemodynamic instability despite intravenous fluids and moderate to “high dose”
vasopressor support OR
Worsening respiratory distress, including pulmonary infiltrates increasing oxygen
requirement including high-flow Oxygen (O2) and/or need for mechanical ventilation
OR
Rapid clinical deterioration
2nd Line Management:
Tocilizumab: IV infusion over 1 hour
Patient weight < 30 kg: 12 mg/kg i.v.
Patient weight ≥ 30 kg: 8 mg/kg i.v. (max dose 800 mg)
Hemodynamic and respiratory support
Lack of clinical improvement while awaiting tocilizumab response
3rd Line Management:
Consider other diagnosis causing clinical deterioration (i.e. sepsis, adrenal insufficiency)
If no improvement with 1st dose of tocilizumab within 12 to 18 hours, consider steroids
(plan rapid taper after hemodynamic normalization):
2 mg/kg methylprednisolone as an initial dose, then 2 mg/kg per day. As steroids are
tapered quickly, monitor for adrenal insufficiency and need for hydrocortisone replacement
If no response to steroids within 24 hours, consider 2nd dose of Tocilizumab (dosed as
above)
Hemodynamic and respiratory support
Lack of clinical improvement while awaiting response to 3rd line management
4th Line Management:
Consider other diagnosis causing clinical deterioration (i.e. sepsis, adrenal insufficiency)
If no response to steroids and 2nd dose of tocilizumab within 24 hours or further
clinical deterioration, consider
siltuximab 11 mg/kg IV over 1 hour
Hemodynamic and respiratory support
Lack of clinical improvement while awaiting response to 4th line management
5th Line Management:
Consider other diagnosis causing clinical deterioration (i.e. sepsis, adrenal insufficiency)
In ongoing CRS despite prior therapy, consider anti-T cell therapies such as
cyclophosphamide, anti-thymocyte globulin, or alemtuzumab
Hemodynamic and respiratory support

### CONCLUSION

Earlier administration of tocilizumab can decrease acute CART19 associated CRS severity (grade, duration of CRS or intensity of medical intervention) and at the same time it will not compromise antitumor efficacy of therapy with CD19 CAR T cells.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

We claim:

1. A composition comprising a JAK-STAT inhibitor (e.g., ruxolitinib), in combination with a CAR therapy (e.g., a CD123 CAR therapy), for use in preventing cytokine release syndrome (CRS), in a subject in need thereof.

2. A method of preventing cytokine release syndrome (CRS) with a CAR therapy (e.g., a CD123 CAR therapy) in a subject in need thereof, comprising administering a JAK-STAT inhibitor (e.g., ruxolitinib), in combination with the CAR therapy, to the subject, thereby preventing CRS in the subject.

3. A composition comprising:
(i) a cell, e.g., a population of immune effector cells, expressing, a chimeric antigen receptor (CAR), wherein the CAR comprises a CD123 binding domain, a transmembrane domain, and an intracellular signaling domain; and
(ii) a JAK-STAT inhibitor, e.g., ruxolitinib,
for use in treating a subject having a disease associated with expression of CD123.

4. A method of treating a subject having a disease associated with expression of CD123, comprising administering to the subject:
(i) a cell, e.g., a population of immune effector cells, expressing a chimeric antigen receptor (CAR), wherein the CAR comprises a CD123 binding domain, a transmembrane domain, and an intracellular signaling domain; and
(ii) a JAK-STAT inhibitor, e.g., ruxolitinib.

5. The method or composition for use of any of the preceding claims, wherein the subject (i) is at risk of developing, has, or is diagnosed with CRS; (ii) is identified or has previously been identified as being at risk for CRS; and/or (iii) has been, is being, or will be administered a CAR therapy, e.g., a CD123 CAR-expressing cell.

6. The method or composition for use of any of the preceding claims, wherein the JAK-STAT inhibitor is chosen from: ruxolitinib, AG490, AZD1480, tofacitinib (tasocitinib or CP-690550), CYT387, fedratinib, baricitinib (INCB039110), lestaurtinib (CEP701), pacritinib (SB1518), XL019, gandotinib (LY2784544), BMS911543, fedratinib (SAR302503), decemotinib (V-509), INCB39110, GEN1, GEN2, GLPG0634, NS018, and N-(cyanomethyl)-4-[2-(4-morpholinoanilino)pyrimidin-4-yl]benzamide, or a pharmaceutically acceptable salt thereof, e.g., wherein the JAK-STAT inhibitor is ruxolitinib or a pharmaceutically acceptable salt thereof.

7. The method or composition for use of any of claim 1-2 or 5-6, wherein the CAR therapy comprises a CD123 CAR-expressing cell.

8. The method or composition for use of any of the preceding claims, further comprising selecting the subject for administration of the JAK-STAT inhibitor (e.g., ruxolitinib).

9. The method or composition for use of any of the preceding claims, wherein the subject is selected based on
(i) his or her risk of developing CRS,
(ii) his or her diagnosis of CRS, and/or
(iii) whether he or she has been, is being, or will be administered a CAR therapy (e.g., CD123 CAR-expressing cell).

10. The method of or composition for use of any of the preceding claims, wherein the subject is selected for administration of the JAK-STAT inhibitor (e.g., ruxolitinib), if the subject is diagnosed with CRS, e.g., severe or non-severe CRS.

11. The method or composition for use of any of the preceding claims, wherein the subject is selected for administration of the JAK-STAT inhibitor (e.g., ruxolitinib), if the subject is at risk of developing CRS.

12. The method or composition for use of any of the preceding claims, wherein the subject is selected for administration of the JAK-STAT inhibitor (e.g., ruxolitinib), if the subject has been, is being, or will be administered a CAR therapy (e.g., CD123 CAR-expressing cell).

13. The method or composition for use of any of the preceding claims, wherein the JAK-STAT inhibitor is ruxolitinib and the CAR therapy is a CD123 CAR-expressing cell.

14. The method or composition for use of any of the preceding claims, wherein the CAR therapy (e.g., CD123 CAR-expressing cell) and the JAK-STAT inhibitor (e.g., ruxolitinib) are administered sequentially.

15. The method or composition for use of any of the preceding claims, wherein the JAK-STAT inhibitor (e.g., ruxolitinib) is administered prior to the CAR therapy (e.g., CD123 CAR-expressing cell).

16. The method or composition for use of any of claims 1-12, wherein the JAK-STAT inhibitor (e.g., ruxolitinib) and the CAR therapy (e.g., CD123 CAR-expressing cell) are administered simultaneously or concurrently.

17. The method or composition for use of any of the preceding claims, wherein the CAR therapy (e.g., CD123 CAR-expressing cell) and the JAK-STAT inhibitor (e.g., ruxolitinib) are administered for a treatment interval, and wherein the treatment interval comprises a single dose of the CAR therapy and multiple doses (e.g., a first and second, and optionally a subsequent dose) of the JAK-STAT inhibitor.

18. The method or composition for use of any of claim 1-15 or 17 wherein the dose of the CAR therapy is administered after (e.g., at least 1 day, 2 days, 3 days, 4 days, 5 days, 6 days, 7 days, 1 week, 2 weeks, 3 weeks, 4 weeks, 5 weeks, or more, after) administration of the first dose of the JAK-STAT inhibitor, e.g., but before administration of the second dose of the inhibitor.

19. The method or composition for use of any of claims 1-13 and 16-17, wherein the dose of the CAR therapy is administered concurrently with (e.g., within 2 days (e.g., within 2 days, 1 day, 24 hours, 12 hours, 6 hours, 4 hours, 2 hours, or less) of), the administration of the first dose of the JAK-STAT inhibitor.

20. The method or composition for use of any of claims 17-19, wherein one or more subsequent doses of the JAK-STAT inhibitor are administered after the second dose of the JAK-STAT inhibitor.

21. The method or composition for use of any of claims 17-20, wherein the doses of the JAK-STAT inhibitor are administered twice a day (BID).

22. The method or composition for use of any of preceding claims, wherein the treatment interval comprises a duration of at least 7 days, e.g., at least 7 days, 8 days, 9 days, 10 days, 1 week, 2 weeks, 3 weeks, 4 weeks, 5 weeks, 6 weeks, 1 month, 2 months, 3 months, 4 months, 5 months, 6 months, 7 months, 8 months, or more.

23. The method or composition for use of any of claims 17-22, wherein the treatment interval is repeated, e.g., one or more times, e.g., 1, 2, 3, 4, or 5 more times, e.g., the treatment interval is followed by one or more, e.g., 1, 2, 3, 4, or 5, subsequent treatment intervals.

24. The method or compositon for use of any of the preceding claims, wherein the CD123 binding domain comprises:
a heavy chain complementary determining region 1 (HC CDR1), a heavy chain complementary determining region 2 (HC CDR2), and a heavy chain complementary determining region 3 (HC CDR3) of any CD123 heavy chain binding domain amino acid sequence listed in Table 12B, Table 11A, or Table 12A; and
a light chain complementary determining region 1 (LC CDR1), a light chain complementary determining region 2 (LC CDR2), and a light chain complementary determining region 3 (LC CDR3) of any CD19 light chain binding domain amino acid sequence listed in Table 12B, Table 11A, or Table 12A.

25. The method or composition for use of any of the preceding claims, wherein the CD123 binding domain comprises a HC CDR1, a HC CDR2, and a HC CDR3 according to the HC CDR amino acid sequences in Tables 5A, 7A, 1A, or 3A, and a LC CDR1, a LC CDR2, and a LC CDR3 according to the LC CDR amino acid sequences in Tables 6A, 8A, 2A or 4A.

26. The method or composition for use of any of the preceding claims, wherein the CD123 binding domain comprises:
i) the amino acid sequence of any heavy chain variable region of a CD123 binding domain listed in Table 12B or 11A;
ii) an amino acid sequence having at least one, two or three modifications but not more than 30, 20 or 10 modifications to the amino acid sequence of any heavy chain variable region of a CD123 binding domain provided in Table 12B or 11A; or
iii) an amino acid sequence with at least 95% identity to the amino acid sequence of any heavy chain variable region of a CD123 binding domain provided in Table 12B or 11A.

27. The method or composition for use of any of the preceding claims, wherein the CD123 binding domain comprises:
(i) the amino acid sequence of any heavy chain of a CD123 binding domain provided in Table 12B, Table 11A, or Table 12A;
(ii) an amino acid sequence having at least one, two or three modifications but not more than 30, 20 or 10 modifications to any heavy chain of a CD123 binding domain provided in Table 12B, Table 11A, or Table 12A; or
(iii) an amino acid sequence with at least 95% identity to the amino acid sequence to any heavy chain of a CD123 binding domain provided in Table 12B, Table 11A, or Table 12A.

28. The method or composition for use of any of the preceding claims, wherein the CD123 binding domain comprises:
(i) the amino acid sequence of any light chain variable region of a CD123 binding domain provided in Table 12B, Table 11A, or Table 12A;
(ii) an amino acid sequence having at least one, two or three modifications but not more than 30, 20 or 10 modifications to the amino acid sequence of any light chain variable region of a CD123 binding domain provided in Table 12B, Table 11A, or Table 12A; or
(iii) an amino acid sequence with at least 95% identity to the amino acid sequence of any light chain variable region of a CD123 binding domain provided in Table 12B, Table 11A, or Table 12A.

29. The method or composition for use of any of the preceding claims, wherein the CD123 binding domain comprises:
(i) the amino acid sequence of any light chain of a CD123 binding domain provided in Table 12B, Table 11A, or Table 12A;
(ii) the amino acid sequence having at least one, two or three modifications but not more than 30, 20 or 10 modifications to any light chain of a CD123 binding domain provided in Table 12B, Table 11A, or Table 12A; or
(iii) an amino acid sequence with at least 95% identity to the amino acid sequence to any light chain of a CD123 binding domain provided in Table 12B, Table 11A, or Table 12A.

30. The method o or composition for use f any of the preceding claims, wherein the CD123 binding domain comprises the amino acid sequence of any heavy chain variable region listed in Table 12B or 11A, and the amino acid sequence of any light chain variable region listed in Table 12B or 11A.

31. The method or composition for use of any of the preceding claims, wherein the CD123 binding domain comprises:
(i) the amino acid sequence selected from a group consisting of SEQ ID NO:480, 483, 485, 478, 158, 159, 160, 157, 217, 218, 219, 216, 276, 277, 278, or 275;
(ii) an amino acid sequence having at least one, two or three modifications but not more than 30, 20 or 10 modifications to any of SEQ ID NO: 480, 483, 485, 478, 158, 159, 160, 157, 217, 218, 219, 216, 276, 277, 278, or 275; or
(iii) an amino acid sequence with at least 95% identity to any of SEQ ID NO: 480, 483, 485, 478, 158, 159, 160, 157, 217, 218, 219, 216, 276, 277, 278, or 275.

32. The method or composition for use of any of the preceding claims, wherein the transmembrane domain comprises a transmembrane domain from a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.

33. The method or composition for use of any of the preceding claims, wherein the transmembrane domain comprises
(i) the amino acid sequence of SEQ ID NO: 6,
(ii) an amino acid sequence comprises at least one, two or three modifications but not more than 20, 10 or 5 modifications of the amino acid sequence of SEQ ID NO:6, or
(iii) a sequence with at least 95% identity to the amino acid sequence of SEQ ID NO:6.

34. The method or composition for use of any of the preceding claims, wherein the CD123 binding domain is connected to the transmembrane domain by a hinge region.

35. The method or composition for use of any of the preceding claims, wherein the hinge region comprises SEQ ID NO:2, or a sequence with at least 95% identity thereof.

36. The method or composition for use of any of the preceding claims, wherein the intracellular signaling domain comprises a costimulatory signaling domain comprising a functional signaling domain obtained from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

37. The method or composition for use of any of the preceding claims, wherein the costimulatory domain comprises the amino acid sequence of SEQ ID NO:7, or an amino acid sequence having at least one, two or three modifications but not more than 20, 10 or 5 modifications of the amino acid sequence of SEQ ID NO:7, or an amino acid sequence with at least 95% identity to the amino acid sequence of SEQ ID NO:7.

38. The method or composition for use of any of the preceding claims, wherein the intracellular signaling domain comprises a functional signaling domain of 4-1BB and/or a functional signaling domain of CD3 zeta.

39. The method or composition for use of any of the preceding claims, wherein the intracellular signaling domain comprises the amino acid sequence of SEQ ID NO: 7 and/or the amino acid sequence of SEQ ID NO:9 or SEQ ID NO:10; or an amino acid sequence having at least one, two or three modifications but not more than 20, 10 or 5 modifications of the amino acid sequence of SEQ ID NO:7 and/or the amino acid sequence of SEQ ID NO:9 or SEQ ID NO:10; or an amino acid sequence with at least 95% identity to the amino acid sequence of SEQ ID NO:7 and/or the amino acid sequence of SEQ ID NO:9 or SEQ ID NO:10.

40. The method or composition for use of any of the preceding claims, wherein the intracellular signaling domain comprises the amino acid sequence of SEQ ID NO:7 and the amino acid sequence of SEQ ID NO:9 or SEQ ID NO:10, wherein the amino acid sequences comprising the intracellular signaling domain are expressed in the same frame and as a single polypeptide chain.

41. The method or composition for use of any of the preceding claims, wherein the CAR further comprises a leader sequence comprising the amino acid sequence of SEQ ID NO:1.

42. The method or composition for use of any of the preceding claims, wherein the CAR comprises:
(i) the amino acid sequence of any of SEQ ID NOs: 99, 100, 101, or 98;
(ii) an amino acid sequence having at least one, two or three modifications but not more than 30, 20 or 10 modifications to any of SEQ ID NOs: 99, 100, 101, or 98; or
(iii) an amino acid sequence with at least 95% identity to any of SEQ ID NOs: 99, 100, 101, or 98.

43. The method or composition for use of any of the preceding claims, wherein the cell comprising a CAR comprises a nucleic acid encoding the CAR.

44. The method or composition of use of claim 43, wherein the nucleic acid encoding the CAR is a lentiviral vector.

45. The method or composition for use of claim 43 or 44, wherein the nucleic acid encoding the CAR is introduced into the cells by lentiviral transduction.

46. The method or composition for use of any of claims 43-45, wherein the nucleic acid encoding the CAR is an RNA, e.g., an in vitro transcribed RNA.

47. The method or composition for use of any of claims 43-46, wherein the nucleic acid encoding the CAR is introduced into the cells by electroporation.

48. The method or composition for use of any of the preceding claims, wherein the cell is a T cell or an NK cell.

49. The method or composition for use of claim 48, wherein the T cell is an autologous or allogeneic T cell.

50. The method or composition for use of any of the preceding claims, wherein the CRS is a severe CRS, e.g., grade 4 or 5 CRS.

51. The method or composition for use of any of claims 1-49, wherein the CRS is a less than severe CRS, e.g., grade 1, 2, or 3 CRS.

52. The method or composition for use of any of the preceding claims, wherein the subject is a mammal, e.g., a human.

53. The method or composition for use of any of the preceding claims, wherein the subject has or is diagnosed with, a disease associated with a B cell antigen, e.g., CD123, e.g., a hematological cancer, e.g., a lymphoma or a leukemia, e.g., acute myeloid leukemia (AML).

54. The method or composition for use of any of the preceding claims, wherein the dose of the CAR therapy (e.g., CD123 CAR therapy) comprises at least about 1×10⁵, 5×10⁶, 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells (e.g., CD123 CAR expressing cells).

55. The method or composition for use of any of the preceding claims, wherein the dose (e.g., each dose) of the JAK-STAT inhibitor (e.g., ruxolitinib) comprises 2.5 mg to 50 mg (e.g., 2.5-5 mg, 5-10 mg, 10-15 mg, 15-20 mg, 20-25 mg, 25-30 mg, 30-35 mg, 35-40 mg, 40-45 mg, or 45-50 mg) of the JAK-STAT inhibitor.

56. A composition comprising a BTK inhibitor (e.g., ibrutinib), alone or in combination with a CAR therapy (e.g., a CD19 CAR therapy, e.g., a CTL019 therapy), for use in preventing cytokine release syndrome (CRS) associated with the CAR therapy, in a subject in need thereof, wherein the subject is identified or has previously been identified as at risk for CRS, thereby preventing CRS in the subject.

57. A method of preventing cytokine release syndrome (CRS), e.g., CRS associated with a CAR therapy (e.g., a CD19 CAR therapy, e.g., a CTL019 therapy) in a subject in need thereof, comprising administering to the subject a BTK inhibitor (e.g., ibrutinib), alone or in combination with the CAR therapy,
wherein the subject is identified or has previously been identified as at risk for CRS, thereby preventing CRS in the subject.

58. The composition for use of claim 56 or the method of claim 57, wherein the subject has been, is being, or will be administered a CAR therapy, e.g., a CD19 CAR therapy, e.g., CTL019.

59. The composition for use of claim 56 or 58, or the method of claims 57-58, further comprising selecting the subject for administration of the BTK inhibitor, e.g., ibrutinib.

60. The composition for use or method of claim 59, wherein the subject is selected based on
(i) his or her risk of developing CRS,
(ii) his or her diagnosis of CRS, and/or
(iii) whether he or she has been, is being, or will be administered a CAR therapy (e.g., a CAR19 therapy, e.g., a CTL019 therapy).

61. The composition for use or method of claim 59 or 60, wherein:
(i) the subject is selected for administration of the BTK inhibitor (e.g., ibrutinib) if the subject is diagnosed with CRS, e.g., severe or non-severe CRS;
(ii) the subject is selected for administration of the BTK inhibitor (e.g., ibrutinib) if the subject is at risk of (e.g., identified as at risk of) developing CRS; or
(iii) the subject is selected for administration of the BTK inhibitor (e.g., ibrutinib) if the subject has been, is being, or will be administered a CAR therapy (e.g., a CAR19 therapy, e.g., a CTL019 therapy).

62. The composition for use or method of any of claims 57-61, wherein the BTK inhibitor is chosen from ibrutinib, GDC-0834, RN-486, CGI-560, CGI-1764, HM-71224, CC-292, ONO-4059, CNX-774, or LFM-A13, or a pharmaceutically acceptable salt thereof, e.g., wherein the BTK inhibitor is ibrutinib or a pharmaceutically acceptable salt thereof.

63. The composition for use or method of any of claims 57-62, wherein CAR therapy is a CAR19 therapy, e.g., a CTL019 therapy.

64. The composition for use or method of any of claims 57-63, wherein the CAR therapy (e.g., CAR19 therapy) and the BTK inhibitor (e.g., ibrutinib) are administered for a treatment interval, and wherein the treatment interval comprises a single dose of the CAR therapy and multiple doses (e.g., a first and second, and optionally a subsequent dose) of the BTK inhibitor.

65. The composition for use or method of any of claims 57-64, wherein the dose of the CAR therapy is administered after (e.g., at least 1 day, 2 days, 3 days, 4 days, 5 days, 6 days, 7 days, 1 week, 2 weeks, 3 weeks, 4 weeks, 5 weeks, or more, after) administration of the first dose of the BTK inhibitor, e.g., but before administration of the second dose of the inhibitor.

66. The composition for use or method of any of claims 57-64, wherein the dose of the CAR therapy is administered concurrently with (e.g., within 2 days (e.g., within 2 days, 1 day, 24 hours, 12 hours, 6 hours, 4 hours, 2 hours, or less) of), the administration of the first dose of the BTK inhibitor.

67. The composition for use or method of any of claims 62-66, wherein one or more subsequent doses of the BTK inhibitor are administered after the second dose of the BTK inhibitor.

68. The composition for use or method of any of claims 57-67, wherein the doses of the BTK inhibitor are administered once a day (QD).

69. The composition for use or method of claim 64-68, wherein the treatment interval comprises a duration of at least 7 days, e.g., at least 7 days, 8 days, 9 days, 10 days, 1 week, 2 weeks, 3 weeks, 4 weeks, 5 weeks, 6 weeks, 1 month, 2 months, 3 months, 4 months, 5 months, 6 months, 7 months, 8 months, or more.

70. The composition for use or method of any of claims 64-69, wherein the treatment interval is repeated, e.g., one or more times, e.g., 1, 2, 3, 4, or 5 more times.

71. The composition for use or method of any of claims 64-70, wherein the treatment interval is followed by one or more, e.g., 1, 2, 3, 4, or 5, subsequent treatment intervals.

72. The composition for use or method of any of claims 64-71, wherein the dose of the CAR therapy (e.g., the CAR19 therapy) comprises at least about 1×10⁵, 5×10⁶, 1×10⁷, 1.5×10⁷, 2×10⁷, 2.5×10⁷, 3×10⁷, 3.5×10⁷, 4×10⁷, 5×10⁷, 1×10⁸, 1.5×10⁸, 2×10⁸, 2.5×10⁸, 3×10⁸, 3.5×10⁸, 4×10⁸, 5×10⁸, 1×10⁹, 2×10⁹, or 5×10⁹ cells (e.g., CD19 CAR-expressing cells).

73. The composition for use or method of any of claims 64-72, wherein the dose (e.g., each dose) of the BTK inhibitor, e.g., ibrutinib (PCI-32765), comprises about 250 mg, 300 mg, 350 mg, 400 mg, 420 mg, 440 mg, 460 mg, 480 mg, 500 mg, 520 mg, 540 mg, 560 mg, 580 mg, 600 mg (e.g., 250 mg, 420 mg or 560 mg) of ibrutinib.

74. The composition for use or method of any of claims 57-73, wherein the CD19 binding domain comprises a heavy chain complementary determining region 1 (HC CDR1), a heavy chain complementary determining region 2 (HC CDR2), and a heavy chain complementary determining region 3 (HC CDR3) of any CD19 heavy chain binding domain amino acid sequence listed in Table 13A or 14A; and a light chain complementary determining region 1 (LC CDR1), a light chain complementary determining region 2 (LC CDR2), and a light chain complementary determining region 3 (LC CDR3) of any CD19 light chain binding domain amino acid sequence listed in Table 13A or 14A.

75. The composition for use or method of any of claims 57-73, wherein the CD19 binding domain comprises a HC CDR1, a HC CDR2, and a HC CDR3 according to the HC CDR amino acid sequences in Table 15A, and a LC CDR1, a LC CDR2, and a LC CDR3 according to the LC CDR amino acid sequences in Table 16A.

76. The composition for use or method of any of claims 57-75, wherein the CD19 binding domain comprises:
(i) the amino acid sequence of any heavy chain variable region of a CD19 binding domain listed in Table 13A or 14A;
(ii) an amino acid sequence having at least one, two or three modifications but not more than 30, 20 or 10 modifications to the amino acid sequence of any heavy chain variable region of a CD19 binding domain provided in Table 13A or 14A; or
(iii) an amino acid sequence with at least 95% identity to the amino acid sequence of any heavy chain variable region of a CD19 binding domain provided in Table 13A or 14A.

77. The composition for use or method of any of claims 57-76, wherein the CD19 binding domain comprises:
(i) the amino acid sequence of any heavy chain of a CD19 binding domain provided in Table 13A or 14A;
(ii) an amino acid sequence having at least one, two or three modifications but not more than 30, 20 or 10 modifications to any heavy chain of a CD19 binding domain provided in Table 13A or 14A; or
(iii) an amino acid sequence with at least 95% identity to the amino acid sequence to any heavy chain of a CD19 binding domain provided in Table 13A or 14A.

78. The composition for use or method of any of claims 57-77, wherein the CD19 binding domain comprises:
(i) the amino acid sequence of any light chain variable region of a CD19 binding domain provided in Table 13A or 14A;
(ii) an amino acid sequence having at least one, two or three modifications but not more than 30, 20 or 10 modifications to the amino acid sequence of any light chain variable region of a CD19 binding domain provided in Table 13A or 14A; or
(iii) an amino acid sequence with at least 95% identity to the amino acid sequence of any light chain variable region of a CD19 binding domain provided in Table 13A or 14A.

79. The composition for use or method of any of claims 57-78, wherein the CD19 binding domain comprises:
(i) the amino acid sequence of any light chain of a CD19 binding domain provided in Table 13A or 14A;
(ii) the amino acid sequence having at least one, two or three modifications but not more than 30, 20 or 10 modifications to any light chain of a CD19 binding domain provided in Table 13A or 14A; or
(iii) an amino acid sequence with at least 95% identity to the amino acid sequence to any light chain of a CD19 binding domain provided in Table 13A or 14A.

80. The composition for use or method of any of claims 57-79, wherein the CD19 binding domain comprises the amino acid sequence of any heavy chain variable region listed in Table 13A or 14A, and the amino acid sequence of any light chain variable region listed in Table 13A or 14A.

81. The composition for use or method of any of claims 57-80, wherein the CD19 binding domain comprises:
(i) the amino acid sequence selected from the group consisting of SEQ ID NO: 774, SEQ ID NO: 710, SEQ ID NO: 711, SEQ ID NO: 712, SEQ ID NO:713, SEQ ID NO:714, SEQ ID NO: 715, SEQ ID NO: 716, SEQ ID NO: 717, SEQ ID NO: 718, SEQ ID NO: 719, SEQ ID NO: 720, SEQ ID NO: 721, SEQ ID NO: 775, SEQ ID NO: 777, or SEQ ID NO: 780;
(i) an amino acid sequence having at least one, two or three modifications but not more than 30, 20 or 10 modifications to any of SEQ ID NO: 774, SEQ ID NO: 710, SEQ ID NO: 711, SEQ ID NO: 712, SEQ ID NO:713, SEQ ID NO:714, SEQ ID NO: 715, SEQ ID NO: 716, SEQ ID NO: 717, SEQ ID NO: 718, SEQ ID NO: 719, SEQ ID NO: 720, SEQ ID NO: 721, SEQ ID NO: 775, SEQ ID NO: 777, or SEQ ID NO: 780; or
(iii) an amino acid sequence with at least 95% identity to the amino acid sequence to any of SEQ ID NO: 774, SEQ ID NO: 710, SEQ ID NO: 711, SEQ ID NO: 712, SEQ ID NO:713, SEQ ID NO:714, SEQ ID NO: 715, SEQ ID NO: 716, SEQ ID NO: 717, SEQ ID NO: 718, SEQ ID NO: 719, SEQ ID NO: 720, SEQ ID NO: 721, SEQ ID NO: 775, SEQ ID NO: 777, or SEQ ID NO: 780.

82. The composition for use or method of any of claims 57-81, wherein the transmembrane domain comprises a transmembrane domain from a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.

83. The composition for use or method of any of claims 57-82, wherein the transmembrane domain comprises
(i) the amino acid sequence of SEQ ID NO: 6,
(ii) an amino acid sequence comprises at least one, two or three modifications but not more than 20, 10 or 5 modifications of the amino acid sequence of SEQ ID NO:6, or
(iii) a sequence with at least 95% identity to the amino acid sequence of SEQ ID NO:6.

84. The composition for use or method of any of claims 57-83, wherein the CD19 binding domain is connected to the transmembrane domain by a hinge region.

85. The composition for use or method of any of claims 57-84, wherein the hinge region comprises SEQ ID NO:2, or a sequence with at least 95% identity thereof.

86. The composition for use or method of any of claims 57-85, wherein the intracellular signaling domain comprises a costimulatory signaling domain comprising a functional signaling domain obtained from a protein selected from the group consisting of a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

87. The composition for use or method of claim 86, wherein the costimulatory domain comprises the amino acid sequence of SEQ ID NO:7, or an amino acid sequence having at least one, two or three modifications but not more than 20, 10 or 5 modifications of the amino acid sequence of SEQ ID NO:7, or an amino acid sequence with at least 95% identity to the amino acid sequence of SEQ ID NO:7.

88. The composition for use or method of claim 86, wherein the intracellular signaling domain comprises a functional signaling domain of 4-1BB and/or a functional signaling domain of CD3 zeta.

89. The composition for use or method of any of claims 86-88, wherein the intracellular signaling domain comprises the amino acid sequence of SEQ ID NO: 7 and/or the amino acid sequence of SEQ ID NO:9 or SEQ ID NO:10; or an amino acid sequence having at least one, two or three modifications but not more than 20, 10 or 5 modifications of the amino acid sequence of SEQ ID NO:7 and/or the amino acid sequence of SEQ ID NO:9 or SEQ ID NO:10; or an amino acid sequence with at least 95% identity to the amino acid sequence of SEQ ID NO:7 and/or the amino acid sequence of SEQ ID NO:9 or SEQ ID NO:10.

90. The composition for use or method of any of claims 86-89, wherein the intracellular signaling domain comprises the amino acid sequence of SEQ ID NO:7 and the amino acid sequence of SEQ ID NO:9 or SEQ ID NO:10, wherein the amino acid sequences comprising the intracellular signaling domain are expressed in the same frame and as a single polypeptide chain.

91. The composition for use or method of any of claims 57-90, wherein the CAR further comprises a leader sequence comprising the amino acid sequence of SEQ ID NO:1.

92. The composition for use or method of any of claims 57-91, wherein the CAR comprises:
(i) the amino acid sequence of any of SEQ ID NO: 773; SEQ ID NO: 758; SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 776, SEQ ID NO: 779, or SEQ ID NO: 781;
(ii) an amino acid sequence having at least one, two or three modifications but not more than 30, 20 or 10 modifications to any of SEQ ID NO: 773; SEQ ID NO: 758; SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 776, SEQ ID NO: 779, or SEQ ID NO: 781; or
(iii) an amino acid sequence with at least 95% identity to any of SEQ ID NO: 773; SEQ ID NO: 758; SEQ ID NO: 759, SEQ ID NO: 760, SEQ ID NO: 761, SEQ ID NO: 762, SEQ ID NO: 763, SEQ ID NO: 764, SEQ ID NO: 765, SEQ ID NO: 766, SEQ ID NO: 767, SEQ ID NO: 768, SEQ ID NO: 769, SEQ ID NO: 776, SEQ ID NO: 779, or SEQ ID NO: 781.

93. The composition for use or method of any of claims 57-92, wherein the cell comprising a CAR comprises a nucleic acid encoding the CAR.

94. The composition for use or method of claim 93, wherein the nucleic acid encoding the CAR is a lentiviral vector.

95. The composition for use or method of claim 93 or 94, wherein the nucleic acid encoding the CAR is introduced into the cells by lentiviral transduction.

96. The composition for use or method of any of claims 93-95, wherein the nucleic acid encoding the CAR is an RNA, e.g., an in vitro transcribed RNA.

97. The composition for use or method of any of claims 93-96, wherein the nucleic acid encoding the CAR is introduced into the cells by electroporation.

98. The composition for use or method of claims 57-97, wherein the cell is a T cell or an NK cell.

99. The composition for use or method of claim 98, wherein the T cell is an autologous or allogeneic T cell.

100. The composition for use or method of any of claims 57-99, wherein the CD19 binding domain is the amino acid sequence of SEQ ID NO: 774; or wherein the CAR comprises the amino acid sequence of SEQ ID NO: 773.

101. The composition for use or method of any of claims 57-100, wherein the CRS is a severe CRS, e.g., grade 4 or 5 CRS.

102. The composition for use or method of any of claims 57-100, wherein the CRS is a less than severe CRS, e.g., grade 1, 2, or 3 CRS.

103. The composition for use or method of any of claims 57-102, wherein the subject has a disease associated with expression of a B cell antigen, e.g., CD19, e.g., a cancer, e.g., a hematological cancer, e.g., a lymphoma or a leukemia, e.g., acute lymphoid leukemia (ALL).

104. The composition for use or method of any of claims 57-103, wherein the subject is a mammal, e.g., a human.

105. The composition for use or method of any of the preceding claims, further comprising administering an IL-6 inhibitor (e.g., an anti-IL6 receptor inhibitor, e.g., an anti-IL6 receptor inhibitor, e.g., tocilizumab), to the subject.

106. The composition for use or method of claim 105, wherein the IL-6 inhibitor is administered prior to, concurrently with, or subsequent to, a dose (e.g., a first dose) of the CAR therapy.

107. The composition for use or method of any of claims 105-106, wherein the IL-6 inhibitor is administered prior to or within 2 weeks (e.g., 2 weeks, 1.5 weeks, 1 week, 14 days, 13 days, 12 days, 11 days, 10 days, 9 days, 8 days, 7 days, 6 days, 5 days, 4 days, 3 days, 2 days, 1 day, 24 hours, 20 hours, 15 hours, 10 hours, 5 hours, 2 hours, 1 hour or less) of a first sign of a symptom of CRS (e.g., a fever, e.g., characterized by a temperature of at least 38° C. (e.g., at least 38.5° C.), e.g., for two successive measurements in 24 hours (e.g., at least 4, 5, 6, 7, 8 hours, or more, apart)) in the subject.

108. The composition for use or method of claim 107, wherein the IL-6 inhibitor is administered after administration of a dose (e.g., a first dose) of the CAR therapy.

109. The composition for use or method of claim 108, wherein the IL-6 inhibitor is administered 1 hour to 10 days (e.g., 1-24 hours, 1-2 hours, 2-4 hours, 4-8 hours, 8-12 hours, 12-24 hours, 1-2 days, 2-3 days, 3-4 days, 4-5 days, 5-7 days, or 7-10 days) after administration of the dose of the CAR therapy.

110. The composition for use or method of any of claims 105-109, comprising administering a dose of tocilizumab of about 5-15 mg/kg, e.g., 8-12 mg/kg (e.g., about 8 mg/kg, about 9 mg/kg, about 10 mg/kg, about 11 mg/kg, or about 12 mg/kg).

111. The composition for use or method of any of claims 105-110, wherein the subject has (e.g., is diagnosed with or identified as having) a high tumor burden prior to treatment with the CAR-therapy, e.g., wherein the high tumor burden is characterized by at least 40% blasts (e.g., at least 40%, 45%, 50%, 60%, 70%, 80%, 90%, 95%, or more, blasts) in a bone marrow of the subject prior to administration of the CAR therapy (e.g., about 1-5 days prior to administration of the CAR therapy).

112. The composition for use or method of any of claims 105-111, wherein the CAR therapy comprises a CD19 CAR-expressing cell, e.g., a CTL-019-expressing cell.

113. An IL-6 inhibitor (e.g., an anti-IL6 receptor inhibitor, e.g., tocilizumab), for use in treating or preventing cytokine release syndrome (CRS) associated with use of a chimeric antigen receptor (CAR) therapy (e.g., a population of cells expressing a CAR in a subject), wherein the IL-6 inhibitor is used prior to, simultaneously with, or within 1 day (e.g., within 24 hours, 12 hours, 6 hours, 5, hours, 4 hours, 3 hours, 2 hours, 1 hour or less) of, use of a dose (e.g., a first dose) of said CAR therapy.

114. A method of treating or preventing cytokine release syndrome (CRS) associated with administration of a chimeric antigen receptor (CAR) therapy (e.g., a population of cells, expressing a CAR) in a subject, comprising administering to the subject an IL-6 inhibitor (e.g., an anti-IL6 receptor inhibitor, e.g., tocilizumab) prior to, simultaneously with, or within 1 day (e.g., within 24 hours, 12 hours, 6 hours, 5, hours, 4 hours, 3 hours, 2 hours, 1 hour or less) of, administration of a dose (e.g., a first dose) of said CAR therapy.

115. The composition for use of claim 113 or the method of claim 114, wherein the IL-6 inhibitor (e.g., tocilizumab) is administered upon (e.g., within 1 hour, 30 minutes, 20 minutes, 15 minutes or less) a first sign of a symptom of CRS (e.g., a fever, e.g., characterized by a temperature of at least 38° C., e.g., for two successive measurements in 24 hours (e.g., at least 4, 5, 6, 7, 8 hours, or more, apart)) in the subject.

116. The composition for use or method, of any of claims 113-115, wherein the CAR comprises an antigen binding domain that binds one or more of the following: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRCSD); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); or immunoglobulin lambda-like polypeptide 1 (IGLL1).

117. The composition for use or method of any of claims 113-115, wherein the antigen recognition domain binds CD19.

118. The composition for use or method of claim 116, wherein the CAR comprises the amino acid sequence of SEQ ID NO: 773.

119. The composition for use or method of any of the preceding claims, wherein the CAR-expressing cell is administered at a dose (e.g., total dose) of 1.5×10⁷ to 5×10⁹ cells per kg (e.g., 0.3×10⁶ to 1×10⁸ cells per kg), e.g., wherein the total dose is administered over multiple doses (e.g., a first dose, a second dose, and optionally a third dose).

120. The composition for use or method of claim 119, wherein the first dose comprises 10% of the total dose (e.g., about 1×10⁷ cells/kg), e.g., administered on a first day.

121. The composition for use or method of claim 120, wherein the second dose comprises 30% of the total dose (e.g., about 3×10⁷ cells/kg), e.g., administered on a subsequent day (e.g., 1, 2, 3, 4, 5, 6, or 7 days after the first dose).

122. The composition for use or method of any of claims 113-121, wherein the IL-6 inhibitor (e.g., tocilizumab) is administered at a dose of about 5-15 mg/kg, e.g., 8-12 mg/kg (e.g., about 8 mg/kg, about 9 mg/kg, about 10 mg/kg, about 11 mg/kg, or about 12 mg/kg).

123. A pharmaceutical composition comprising (i) a population of immune effector cells, expressing a chimeric antigen receptor (CAR), wherein the CAR comprises a CD123 binding domain, a transmembrane domain, and an intracellular signaling domain; and (ii) a JAK-STAT inhibitor, e.g., ruxolitinib.

124. The pharmaceutical composition of claim 123, wherein the composition further comprises an IL-6 inhibitor (e.g., an anti-IL6 receptor inhibitor, e.g., tocilizumab).

125. A pharmaceutical composition comprising (i) a CD123 chimeric antigen receptor (CAR) therapy (e.g., a population of immune effector cells expressing a CAR, wherein the CAR comprises a CD123 binding domain, a transmembrane domain, and an intracellular signaling domain); and (ii) a JAK-STAT inhibitor, e.g., ruxolitinib, for use in treating a cancer or for use in preventing cytokine release syndrome (CRS).

126. The pharmaceutical composition of claim 125, wherein the composition for use further comprises an IL-6 inhibitor (e.g., an anti-IL6 receptor inhibitor, e.g., tocilizumab).

127. A pharmaceutical composition comprising (i) a BTK inhibitor (e.g., ibrutinib); and (ii) a chimeric antigen receptor (CAR) therapy (e.g., a CD19 CAR-therapy, e.g., a CTL019 therapy); for use in preventing cytokine release syndrome (CRS), e.g., in a subject that is identified or has previously been identified as at risk for CRS.

128. The pharmaceutical composition of claim 127, wherein the composition further comprises an IL-6 inhibitor (e.g., an anti-IL6 receptor inhibitor, e.g., tocilizumab).
